SEQUENCE LISTING (344 sequences)

SEQ ID NO: 1 - DNA, 2910 bp, Neisseria gonorrhoeae
tgaaatgatagagtcaagtgaataagtgcatcaggcggatgccttggcgatgataggcgacgaaggacgtgtaagcctgcgaaaagcgcgggggagctggcaataaagcaatgatcccgcggtgtccgaatggggaaacccactgcattctgtgcagtatcctaagttgaatacataggcttagagaagcgaacccggagaactgaaccatctaagtacccggaggaaaagaaatcaaccgagattccgcaagtagtggcgagcgaacgcggaggagcctgtacgtaataactgtcgaggtagaagaacaagctgggaagcttgaccatagcgggtgacagtcccgtattcgaaatctcaacagcggtactaagcgtacgaaaagtagggcgggacacgtgaaatcctgtctgaatatggggggaccatcctccaaggctaaatactcatcatcgaccgatagtgaaccagtaccgtgagggaaaggcgaaaagaaccccgggaggggagtgaaacagaacctgaaacctgatgcatacaaacagtgggagcgccctagtggtgtgactgcgtaccttttgtataatgggtcaacgacttacattcagtagcgagcttaaccggataggggaggcgtagggaaaccgagtcttaatagggcgatgagttgctgggtgtagacccgaaaccgagtgatctatccatggccaggttgaaggtgccgtaacaggtactggaggaccgaacccacgcatgttgcaaaatgcggggatgagctgtgggtaggggtgaaaggctaaacaaactcggagatagctggttctccccgaaaactatttaggtagtgcctcgagcaagacactgatgggggtaaagcactgttatggctagggggttattgcaacttaccaacccatggcaaactcagaataccatcaagtggttcctcgggagacagacagcgggtgctaacgtccgttgtcaagagggaaacaacccagaccgccggctaaggtcccaaatgatagattaagtggtaaacgaagtgggaaggcacagacagccaggatgttggcttagaagcagccatcatttaaagaaagcgtaatagctcactggtcgagtcgtcctgcgcggaagatgtaacggggctcaaatctataaccgaagctgcggatgccggtttaccggcatggtaggggagcgttctgtaggctgatgaaggtgcattgtaaagtgtgctggaggtatcagaagtgcgaatgttgacatgagtagcgataaagcgggtgaaaagcccgctcgccgaaagcccaaggtttcctacgcaacgttcatcggcgtagggtgagtcggcccctaaggcgaggcagaaatgcgtagtcgatgggaaacaggttaatattcctgtacttgattcaaatgcgatgtggggacggagaaggttaggttggcaagctgttggaatagcttgtttaagccggtaggtggaagacttaggcaaatccgggttttcttaacaccgaagaagtgatgacgagtgtttacggacacgaagcaaccgataccacgcttccaggaaaagccactaagcttcagtttgaatcgaaccgtaccgcaaaccgacacaggtgggcaggatgagaattctaaggcgcttgagagaactcgggagaaggaactcggcaaattgataccgtaacttcgggagaaggtatgccctctaaggttaaggacttgctccgtaagccccggagggtcgcagagaataggtggctgcgacttgtttattaaaaacacgagcactcttgccaacacgaaagtggacgtatagggtgtaacgcctgcccggtgccggaaggttaattgaagatgtgcaagcatcggatcgaagccccggtaaacggcggccgtaactataacggtcctaaggtagcgaaattccttgtcgggtaagttccgacccgcacgaatggcgtaacgatggccacactgtctcctcccgagactcagcgaagttgaagtggttgtgaagatgcaatctacccgctgctagacggaaagaccccgtgaacctttactgtagctttgcattggactttgaagtcacttgtgtaggataggtggaaggcttggaagcaaagacgccagtctctgtggagtcgtccttgaaaataccaccctggtgtctttgaggttctaacccagacccgtcatccgggtcggggaccgtgcatggtaggcagtttgactggggcggtctcctcccaaagcgtaacggaggagttcgaaggttacctaggtccggtcggaaatcggactgatagtgcaatggcaaaaggtagcttaactgcgagaccgacaagtcgggcaggtgcgaaagcaggacatagtgatccggtggttctgtatggaagggccatcgctcaacggataaaaggtactccggggataacaggcttgattccgcccaagagttcatatcgacggcggagtttggcacctcgatgtcggctcatcacatcctggggctgtagtcggtcccaagggtatggctgttcgccattttaaagtggtacgtgagttgggtttaaaacgtcgtgagacagtttggtccctatctgcagtgggcgttggaagtttgacgggggctgctcctagtacgagaggaccggagtggacgaacctctggtgtaccggttgtaacgccagttgcatagccgggtagctaagttcggaagagataagcgctgaaagcatctaagcgcgaaactcgcctgaagatgagacttcccttgcggtttaaccgcactaaagggtcgttcgagaccaggacgttgataggtggggtgtggaagcgcggtaacgcgtgaagctaacccatactaattgcccgtgaggcttgactct

SEQ ID NO: 2 - DNA, 2910 bp, Neisseria gonorrhoeae
agagtcaagcctcacgggcaattagtatgggttagcttcacgcgttaccgcgcttccacaccccacctatcaacgtcctggtctcgaacgaccctttagtgcggttaaaccgcaagggaagtctcatcttcaggcgagtttcgcgcttagatgctttcagcgcttatctcttccgaacttagctacccggctatgcaactggcgttacaaccggtacaccagaggttcgtccactccggtcctctcgtactaggagcagcccccgtcaaacttccaacgcccactgcagatagggaccaaactgtctcacgacgttttaaacccaactcacgtaccactttaaaatggcgaacagccatacccttgggaccgactacagccccaggatgtgatgagccgacatcgaggtgccaaactccgccgtcgatatgaactcttgggcggaatcaagcctgttatccccggagtaccttttatccgttgagcgatggcccttccatacagaaccaccggatcactatgtcctgctttcgcacctgcccgacttgtcggtctcgcagttaagctaccttttgccattgcactatcagtccgatttccgaccggacctaggtaaccttcgaactcctccgttacgctttgggaggagaccgccccagtcaaactgcctaccatgcacggtccccgacccggatgacgggtctgggttagaacctcaaagacaccagggtggtattttcaaggacgactccacagagactggcgtctttgcttccaagccttccacctatcctacacaagtgacttcaaagtccaatgcaaagctacagtaaaggttcacggggtctttccgtctagcagcgggtagattgcatcttcacaaccacttcaacttcgctgagtctcgggaggagacagtgtggccatcgttacgccattcgtgcgggtcggaacttacccgacaaggaatttcgctaccttaggaccgttatagttacggccgccgtttaccggggcttcgatccgatgcttgcacatcttcaattaaccttccggcaccgggcaggcgttacaccctatacgtccactttcgtgttggcaagagtgctcgtgtttttaataaacaagtcgcagccacctattctctgcgaccctccggggcttacggagcaagtccttaaccttagagggcataccttctcccgaagttacggtatcaatttgccgagttccttctcccgagttctctcaagcgccttagaattctcatcctgcccacctgtgtcggtttgcggtacggttcgattcaaactgaagcttagtggcttttcctggaagcgtggtatcggttgcttcgtgtccgtaaacactcgtcatcacttcttcggtgttaagaaaacccggatttgcctaagtcttccacctaccggcttaaacaagctattccaacagcttgccaacctaaccttctccgtccccacatcgcatttgaatcaagtacaggaatattaacctgtttcccatcgactacgcatttctgcctcgccttaggggccgactcaccctacgccgatgaacgttgcgtaggaaaccttgggctttcggcgagcgggcttttcacccgctttatcgctactcatgtcaacattcgcacttctgatacctccagcacactttacaatgcaccttcatcagcctacagaacgctcccctaccatgccggtaaaccggcatccgcagcttcggttatagatttgagccccgttacatcttccgcgcaggacgactcgaccagtgagctattacgctttctttaaatgatggctgcttctaagccaacatcctggctgtctgtgccttcccacttcgtttaccacttaatctatcatttgggaccttagccggcggtctgggttgtttccctcttgacaacggacgttagcacccgctgtctgtctcccgaggaaccacttgatggtattctgagtttgccatgggttggtaagttgcaataaccccctagccataacagtgctttacccccatcagtgtcttgctcgaggcactacctaaatagttttcggggagaaccagctatctccgagtttgtttagcctttcacccctacccacagctcatccccgcattttgcaacatgcgtgggttcggtcctccagtacctgttacggcaccttcaacctggccatggatagatcactcggtttcgggtctacacccagcaactcatcgccctattaagactcggtttccctacgcctcccctatccggttaagctcgctactgaatgtaagtcgttgacccattatacaaaaggtacgcagtcacaccactagggcgctcccactgtttgtatgcatcaggtttcaggttctgtttcactcccctcccggggttcttttcgcctttccctcacggtactggttcactatcggtcgatgatgagtatttagccttggaggatggtccccccatattcagacaggatttcacgtgtcccgccctacttttcgtacgcttagtaccgctgttgagatttcgaatacgggactgtcacccgctatggtcaagcttcccagcttgttcttctacctcgacagttattacgtacaggctcctccgcgttcgctcgccactacttgcggaatctcggttgatttcttttcctccgggtacttagatggttcagttctccgggttcgcttctctaagcctatgtattcaacttaggatactgcacagaatgcagtgggtttccccattcggacaccgcgggatcattgctttattgccagctcccccgcgcttttcgcaggcttacacgtccttcgtcgcctatcatcgccaaggcatccgcctgatgcacttattcacttgactctatcatttca

SEQ ID NO: 3 - RNA, 2910 nt, Neisseria gonorrhoeae
ugaaaugauagagucaagugaauaagugcaucaggcggaugccuuggcgaugauaggcgacgaaggacguguaagccugcgaaaagcgcgggggagcuggcaauaaagcaaugaucccgcgguguccgaauggggaaacccacugcauucugugcaguauccuaaguugaauacauaggcuuagagaagcgaacccggagaacugaaccaucuaaguacccggaggaaaagaaaucaaccgagauuccgcaaguaguggcgagcgaacgcggaggagccuguacguaauaacugucgagguagaagaacaagcugggaagcuugaccauagcgggugacagucccguauucgaaaucucaacagcgguacuaagcguacgaaaaguagggcgggacacgugaaauccugucugaauauggggggaccauccuccaaggcuaaauacucaucaucgaccgauagugaaccaguaccgugagggaaaggcgaaaagaaccccgggaggggagugaaacagaaccugaaaccugaugcauacaaacagugggagcgcccuaguggugugacugcguaccuuuuguauaaugggucaacgacuuacauucaguagcgagcuuaaccggauaggggaggcguagggaaaccgagucuuaauagggcgaugaguugcuggguguagacccgaaaccgagugaucuauccauggccagguugaaggugccguaacagguacuggaggaccgaacccacgcauguugcaaaaugcggggaugagcuguggguaggggugaaaggcuaaacaaacucggagauagcugguucuccccgaaaacuauuuagguagugccucgagcaagacacugauggggguaaagcacuguuauggcuaggggguuauugcaacuuaccaacccauggcaaacucagaauaccaucaagugguuccucgggagacagacagcgggugcuaacguccguugucaagagggaaacaacccagaccgccggcuaaggucccaaaugauagauuaagugguaaacgaagugggaaggcacagacagccaggauguuggcuuagaagcagccaucauuuaaagaaagcguaauagcucacuggucgagucguccugcgcggaagauguaacggggcucaaaucuauaaccgaagcugcggaugccgguuuaccggcaugguaggggagcguucuguaggcugaugaaggugcauuguaaagugugcuggagguaucagaagugcgaauguugacaugaguagcgauaaagcgggugaaaagcccgcucgccgaaagcccaagguuuccuacgcaacguucaucggcguagggugagucggccccuaaggcgaggcagaaaugcguagucgaugggaaacagguuaauauuccuguacuugauucaaaugcgauguggggacggagaagguuagguuggcaagcuguuggaauagcuuguuuaagccgguagguggaagacuuaggcaaauccggguuuucuuaacaccgaagaagugaugacgaguguuuacggacacgaagcaaccgauaccacgcuuccaggaaaagccacuaagcuucaguuugaaucgaaccguaccgcaaaccgacacaggugggcaggaugagaauucuaaggcgcuugagagaacucgggagaaggaacucggcaaauugauaccguaacuucgggagaagguaugcccucuaagguuaaggacuugcuccguaagccccggagggucgcagagaauagguggcugcgacuuguuuauuaaaaacacgagcacucuugccaacacgaaaguggacguauaggguguaacgccugcccggugccggaagguuaauugaagaugugcaagcaucggaucgaagccccgguaaacggcggccguaacuauaacgguccuaagguagcgaaauuccuugucggguaaguuccgacccgcacgaauggcguaacgauggccacacugucuccucccgagacucagcgaaguugaagugguugugaagaugcaaucuacccgcugcuagacggaaagaccccgugaaccuuuacuguagcuuugcauuggacuuugaagucacuuguguaggauagguggaaggcuuggaagcaaagacgccagucucuguggagucguccuugaaaauaccacccuggugucuuugagguucuaacccagacccgucauccgggucggggaccgugcaugguaggcaguuugacuggggcggucuccucccaaagcguaacggaggaguucgaagguuaccuagguccggucggaaaucggacugauagugcaauggcaaaagguagcuuaacugcgagaccgacaagucgggcaggugcgaaagcaggacauagugauccggugguucuguauggaagggccaucgcucaacggauaaaagguacuccggggauaacaggcuugauuccgcccaagaguucauaucgacggcggaguuuggcaccucgaugucggcucaucacauccuggggcuguagucggucccaaggguauggcuguucgccauuuuaaagugguacgugaguuggguuuaaaacgucgugagacaguuuggucccuaucugcagugggcguuggaaguuugacgggggcugcuccuaguacgagaggaccggaguggacgaaccucugguguaccgguuguaacgccaguugcauagccggguagcuaaguucggaagagauaagcgcugaaagcaucuaagcgcgaaacucgccugaagaugagacuucccuugcgguuuaaccgcacuaaagggucguucgagaccaggacguugauaggugggguguggaagcgcgguaacgcgugaagcuaacccauacuaauugcccgugaggcuugacucu

SEQ ID NO: 4 - DNA, 1545 bp, Neisseria gonorrhoeae
tgaacataagagtttgatcctggctcagattgaacgctggcggcatgctttacacatgcaagtcggacggcagcacagggaagcttgcttctcgggtggcgagtggcgaacgggtgagtaacatatcggaacgtaccgggtagcgggggataactgatcgaaagatcagctaataccgcatacgtcttgagagggaaagcaggggaccttcgggccttgcgctatccgagcggccgatatctgattagctggttggcggggtaaaggcccaccaaggcgacgatcagtagcgggtctgagaggatgatccgccacactgggactgagacacggcccagactcctacgggaggcagcagtggggaattttggacaatgggcgcaagcctgatccagccatgccgcgtgtctgaagaaggccttcgggttgtaaaggacttttgtcagggaagaaaaggccgttgccaatatcggcggccgatgacggtacctgaagaataagcaccggctaactacgtgccagcagccgcggtaatacgtagggtgcgagcgttaatcggaattactgggcgtaaagcgggcgcagacggttacttaagcaggatgtgaaatccccgggctcaacccgggaactgcgttctgaactgggtgactcgagtgtgtcagagggaggtggaattccacgtgtagcagtgaaatgcgtagagatgtggaggaataccgatggcgaaggcagcctcctgggataacactgacgttcatgtccgaaagcgtgggtagcaaacaggattagataccctggtagtccacgccctaaacgatgtcaattagctgttgggcaacttgattgcttggtagcgtagctaacgcgtgaaattgaccgcctggggagtacggtcgcaagattaaaactcaaaggaattgacggggacccgcacaagcggtggatgatgtggattaattcgatgcaacgcgaagaaccttacctggttttgacatgtgcggaatcctccggagacggaggagtgccttcgggagccgtaacacaggtgctgcatggctgtcgtcagctcgtgtcgtgagatgttgggttaagtcccgcaacgagcgcaacccttgtcattagttgccatcattcggttgggcactctaatgagactgccggtgacaagccggaggaaggtggggatgacgtcaagtcctcatggcccttatgaccagggcttcacacgtcatacaatggtcggtacagagggtagccaagccgcgaggcggagccaatctcacaaaaccgatcgtagtccggattgcactctgcaactcgagtgcatgaagtcggaatcgctagtaatcgcaggtcagcatactgcggtgaatacgttcccgggtcttgtacacaccgcccgtcacaccatgggagtgggggataccagaagtaggtagggtaaccgcaaggagtccgcttaccacggtatgcttcatgactggggtgaagtcgtaacaaggtagccgtaggggaacctgcggctggatcacctcctttcta

SEQ ID NO: 5 - DNA, 1545 bp, Neisseria gonorrhoeae
tagaaaggaggtgatccagccgcaggttcccctacggctaccttgttacgacttcaccccagtcatgaagcataccgtggtaagcggactccttgcggttaccctacctacttctggtatcccccactcccatggtgtgacgggcggtgtgtacaagacccgggaacgtattcaccgcagtatgctgacctgcgattactagcgattccgacttcatgcactcgagttgcagagtgcaatccggactacgatcggttttgtgagattggctccgcctcgcggcttggctaccctctgtaccgaccattgtatgacgtgtgaagccctggtcataagggccatgaggacttgacgtcatccccaccttcctccggcttgtcaccggcagtctcattagagtgcccaaccgaatgatggcaactaatgacaagggttgcgctcgttgcgggacttaacccaacatctcacgacacgagctgacgacagccatgcagcacctgtgttacggctcccgaaggcactcctccgtctccggaggattccgcacatgtcaaaaccaggtaaggttcttcgcgttgcatcgaattaatccacatcatccaccgcttgtgcgggtccccgtcaattcctttgagttttaatcttgcgaccgtactccccaggcggtcaatttcacgcgttagctacgctaccaagcaatcaagttgcccaacagctaattgacatcgtttagggcgtggactaccagggtatctaatcctgtttgctacccacgctttcggacatgaacgtcagtgttatcccaggaggctgccttcgccatcggtattcctccacatctctacgcatttcactgctacacgtggaattccacctccctctgacacactcgagtcacccagttcagaacgcagttcccgggttgagcccggggatttcacatcctgcttaagtaaccgtctgcgcccgctttacgcccagtaattccgattaacgctcgcaccctacgtattaccgcggctgctggcacgtagttagccggtgcttattcttcaggtaccgtcatcggccgccgatattggcaacggccttttcttccctgacaaaagtcctttacaacccgaaggccttcttcagacacgcggcatggctggatcaggcttgcgcccattgtccaaaattccccactgctgcctcccgtaggagtctgggccgtgtctcagtcccagtgtggcggatcatcctctcagacccgctactgatcgtcgccttggtgggcctttaccccgccaaccagctaatcagatatcggccgctcggatagcgcaaggcccgaaggtcccctgctttccctctcaagacgtatgcggtattagctgatctttcgatcagttatcccccgctacccggtacgttccgatatgttactcacccgttcgccactcgccacccgagaagcaagcttccctgtgctgccgtccgacttgcatgtgtaaagcatgccgccagcgttcaatctgagccaggatcaaactcttatgttca

SEQ ID NO: 6 - RNA, 1545 nt, Neisseria gonorrhoeae
ugaacauaagaguuugauccuggcucagauugaacgcuggcggcaugcuuuacacaugcaagucggacggcagcacagggaagcuugcuucucggguggcgaguggcgaacgggugaguaacauaucggaacguaccggguagcgggggauaacugaucgaaagaucagcuaauaccgcauacgucuugagagggaaagcaggggaccuucgggccuugcgcuauccgagcggccgauaucugauuagcugguuggcgggguaaaggcccaccaaggcgacgaucaguagcgggucugagaggaugauccgccacacugggacugagacacggcccagacuccuacgggaggcagcaguggggaauuuuggacaaugggcgcaagccugauccagccaugccgcgugucugaagaaggccuucggguuguaaaggacuuuugucagggaagaaaaggccguugccaauaucggcggccgaugacgguaccugaagaauaagcaccggcuaacuacgugccagcagccgcgguaauacguagggugcgagcguuaaucggaauuacugggcguaaagcgggcgcagacgguuacuuaagcaggaugugaaauccccgggcucaacccgggaacugcguucugaacugggugacucgagugugucagagggagguggaauuccacguguagcagugaaaugcguagagauguggaggaauaccgauggcgaaggcagccuccugggauaacacugacguucauguccgaaagcguggguagcaaacaggauuagauacccugguaguccacgcccuaaacgaugucaauuagcuguugggcaacuugauugcuugguagcguagcuaacgcgugaaauugaccgccuggggaguacggucgcaagauuaaaacucaaaggaauugacggggacccgcacaagcgguggaugauguggauuaauucgaugcaacgcgaagaaccuuaccugguuuugacaugugcggaauccuccggagacggaggagugccuucgggagccguaacacaggugcugcauggcugucgucagcucgugucgugagauguuggguuaagucccgcaacgagcgcaacccuugucauuaguugccaucauucgguugggcacucuaaugagacugccggugacaagccggaggaagguggggaugacgucaaguccucauggcccuuaugaccagggcuucacacgucauacaauggucgguacagaggguagccaagccgcgaggcggagccaaucucacaaaaccgaucguaguccggauugcacucugcaacucgagugcaugaagucggaaucgcuaguaaucgcaggucagcauacugcggugaauacguucccgggucuuguacacaccgcccgucacaccaugggagugggggauaccagaaguagguaggguaaccgcaaggaguccgcuuaccacgguaugcuucaugacuggggugaagucguaacaagguagccguaggggaaccugcggcuggaucaccuccuuucua

SEQ ID NO: 7 - DNA, 2904 bp, Neisseria gonorrhoeae
tgaaatgatagagtcaagtgaataagtgcatcaggcggatgccttggcgatgataggcgacgaaggacgtgtaagcctgcgaaaagcgcgggggagctggcaataaagcaatgatcccgcggtgtccgaatggggaaacccactgcattctgtgcagtatcctaagttgaatacataggcttagagaagcgaacccggagaactgaaccatctaagtacccggaggaaaagaaatcaaccgagattccgcaagtagtggcgagcgaacgcggaggagcctgtacgtaataactgtcgaggtagaagaacaagctgggaagcttgaccatagcgggtgacagtcccgtattcgaaatctcaacagcggtactaagcgtacgaaaagtagggcgggacacgtgaaatcctgtctgaatatggggggaccatcctccaaggctaaatactcatcatcgaccgatagtgaaccagtaccgtgagggaaaggcgaaaagaaccccgggaggggagtgaaacagaacctgaaacctgatgcatacaaacagtgggagcgccctagtggtgtgactgcgtaccttttgtataatgggtcaacgacttacattcagtagcgagcttaaccggataggggaggcgtagggaaaccgagtcttaatagggcgatgagttgctgggtgtagacccgaaaccgagtgatctatccatggccaggttgaaggtgccgtaacaggtactggaggaccgaacccacgcatgttgcaaaatgcggggatgagctgtgggtaggggtgaaaggctaaacaaactcggagatagctggttctccccgaaaactatttaggtagtgcctcgagcaagacactgatgggggtaaagcactgttatggctagggggttattgcaacttaccaacccatggcaaactcagaataccatcaagtggttcctcgggagacagacagcgggtgctaacgtccgttgtcaagagggaaacaacccagaccgccggctaaggtcccaaatgatagattaagtggtaaacgaagtgggaaggcacagacagccaggatgttggcttagaagcagccatcatttaaagaaagcgtaatagctcactggtcgagtcgtcctgcgcggaagatgtaacggggctcaaatctataacccaagctgcgtatgccggtttaccggcatggtaggggagcgttctgtaggctgatgaaggtgcattgtaaagtgtgctggaggtatcagaagtgcgaatgttgacatgagtagcgataaagcgggtgaaaagcccgctcgccgcaaagcccaaggtttcctacgcaacgttcatcggcgtagggtgagtcggcccctaaggcgaggcagaaatgcgtagtcgatgggaaacaggttaatattcctgtacttgattcaaatgcgatgtggggacggagaaggttaggttggcaagctgttggaatagcttgtttaagccggtaggtggaagacttaggcaaatccgggttttcttaacaccgagaagtgatgacgagtgtctacggacacgaagcaaccgataccacgcttccaggaaaagccactaagcttcagtttgaatcgaaccgtaccgcaaaccgacacaggtgggcaggatgagaattctaaggcgcttgagagaactcgggagaaggaactcggcaaattgataccgtaacttcgggagaaggtatgccctctaaggttaaggacttgctccgtaagccccggagggtcgcagagaataggtggctgcgactgtttattaaaaacacagcactctgccaacacgaaagtggacgtatagggtgtgacgcctgcccggtgccggaaggttaattgaagatgtgcaagcatcggatcgaagccccggtaaacggcggccgtaactataacggtcctaaggtagcgaaattccttgtcgggtaagttccgacccgcacgaatggcgtaacgatggccacactgtctcctcccgagactcagcgaagttgaagtggttgtgaagatgcaatctacccgctgctagacggaaagaccccgtgaacctttactgtagctttgcattggactttgaagtcacttgtgtaggataggtgggaggcttggaagcagagacgccagtctctgtggagtcgtccttgaaataccaccctggtgtctttgaggttctaacccagacccgtcatccgggtcggggaccgtgcatggtaggcagtttgactggggcggtctcctcccaaagcgtaacggaggagttcgaaggttacctaggtccggtcggaaatcggactgatagtgcaatggcaaaaggtagcttaactgcgagaccgacaagtcgggcaggtgcgaaagcaggacatagtgatccggtggttctgtatggaagggccatcgctcaacggataaaaggtactccggggataacaggctgattccgcccaagagttcatatcgacggcggagtttggcacctcgatgtcggctcatcacatcctggggctgtagtcggtcccaagggtatggctgttcgccatttaaagtggtacgtgagctgggtttaaaacgtcgtgagacagtttggtccctatctgcagtgggcgttggaagtttgacgggggctgctcctagtacgagaggaccggagtggacgaacctctggtgtaccggttgtaacgccagttgcatagccgggtagctaagttcggaagagataagcgctgaaagcatctaagcgcgaaactcgcctgaagatgagacttcccttgcggtttaaccgcactaaagggtcgttcgagaccaggacgttgataggtggggtgtggaagcgcggtaacgcgtgaagctaacccatactaattgcccgtgaggcttgactct

SEQ ID NO: 8 - DNA, 2904 bp, Neisseria gonorrhoeae
agagtcaagcctcacgggcaattagtatgggttagcttcacgcgttaccgcgcttccacaccccacctatcaacgtcctggtctcgaacgaccctttagtgcggttaaaccgcaagggaagtctcatcttcaggcgagtttcgcgcttagatgctttcagcgcttatctcttccgaacttagctacccggctatgcaactggcgttacaaccggtacaccagaggttcgtccactccggtcctctcgtactaggagcagcccccgtcaaacttccaacgcccactgcagatagggaccaaactgtctcacgacgttttaaacccagctcacgtaccactttaaatggcgaacagccatacccttgggaccgactacagccccaggatgtgatgagccgacatcgaggtgccaaactccgccgtcgatatgaactcttgggcggaatcagcctgttatccccggagtaccttttatccgttgagcgatggcccttccatacagaaccaccggatcactatgtcctgctttcgcacctgcccgacttgtcggtctcgcagttaagctaccttttgccattgcactatcagtccgatttccgaccggacctaggtaaccttcgaactcctccgttacgctttgggaggagaccgccccagtcaaactgcctaccatgcacggtccccgacccggatgacgggtctgggttagaacctcaaagacaccagggtggtatttcaaggacgactccacagagactggcgtctctgcttccaagcctcccacctatcctacacaagtgacttcaaagtccaatgcaaagctacagtaaaggttcacggggtctttccgtctagcagcgggtagattgcatcttcacaaccacttcaacttcgctgagtctcgggaggagacagtgtggccatcgttacgccattcgtgcgggtcggaacttacccgacaaggaatttcgctaccttaggaccgttatagttacggccgccgtttaccggggcttcgatccgatgcttgcacatcttcaattaaccttccggcaccgggcaggcgtcacaccctatacgtccactttcgtgttggcagagtgctgtgtttttaataaacagtcgcagccacctattctctgcgaccctccggggcttacggagcaagtccttaaccttagagggcataccttctcccgaagttacggtatcaatttgccgagttccttctcccgagttctctcaagcgccttagaattctcatcctgcccacctgtgtcggtttgcggtacggttcgattcaaactgaagcttagtggcttttcctggaagcgtggtatcggttgcttcgtgtccgtagacactcgtcatcacttctcggtgttaagaaaacccggatttgcctaagtcttccacctaccggcttaaacaagctattccaacagcttgccaacctaaccttctccgtccccacatcgcatttgaatcaagtacaggaatattaacctgtttcccatcgactacgcatttctgcctcgccttaggggccgactcaccctacgccgatgaacgttgcgtaggaaaccttgggctttgcggcgagcgggcttttcacccgctttatcgctactcatgtcaacattcgcacttctgatacctccagcacactttacaatgcaccttcatcagcctacagaacgctcccctaccatgccggtaaaccggcatacgcagcttgggttatagatttgagccccgttacatcttccgcgcaggacgactcgaccagtgagctattacgctttctttaaatgatggctgcttctaagccaacatcctggctgtctgtgccttcccacttcgtttaccacttaatctatcatttgggaccttagccggcggtctgggttgtttccctcttgacaacggacgttagcacccgctgtctgtctcccgaggaaccacttgatggtattctgagtttgccatgggttggtaagttgcaataaccccctagccataacagtgctttacccccatcagtgtcttgctcgaggcactacctaaatagttttcggggagaaccagctatctccgagtttgtttagcctttcacccctacccacagctcatccccgcattttgcaacatgcgtgggttcggtcctccagtacctgttacggcaccttcaacctggccatggatagatcactcggtttcgggtctacacccagcaactcatcgccctattaagactcggtttccctacgcctcccctatccggttaagctcgctactgaatgtaagtcgttgacccattatacaaaaggtacgcagtcacaccactagggcgctcccactgtttgtatgcatcaggtttcaggttctgtttcactcccctcccggggttcttttcgcctttccctcacggtactggttcactatcggtcgatgatgagtatttagccttggaggatggtccccccatattcagacaggatttcacgtgtcccgccctacttttcgtacgcttagtaccgctgttgagatttcgaatacgggactgtcacccgctatggtcaagcttcccagcttgttcttctacctcgacagttattacgtacaggctcctccgcgttcgctcgccactacttgcggaatctcggttgatttcttttcctccgggtacttagatggttcagttctccgggttcgcttctctaagcctatgtattcaacttaggatactgcacagaatgcagtgggtttccccattcggacaccgcgggatcattgctttattgccagctcccccgcgcttttcgcaggcttacacgtccttcgtcgcctatcatcgccaaggcatccgcctgatgcacttattcacttgactctatcatttca

SEQ ID NO: 9 - RNA, 2904 nt, Neisseria gonorrhoeae
ugaaaugauagagucaagugaauaagugcaucaggcggaugccuuggcgaugauaggcgacgaaggacguguaagccugcgaaaagcgcgggggagcuggcaauaaagcaaugaucccgcgguguccgaauggggaaacccacugcauucugugcaguauccuaaguugaauacauaggcuuagagaagcgaacccggagaacugaaccaucuaaguacccggaggaaaagaaaucaaccgagauuccgcaaguaguggcgagcgaacgcggaggagccuguacguaauaacugucgagguagaagaacaagcugggaagcuugaccauagcgggugacagucccguauucgaaaucucaacagcgguacuaagcguacgaaaaguagggcgggacacgugaaauccugucugaauauggggggaccauccuccaaggcuaaauacucaucaucgaccgauagugaaccaguaccgugagggaaaggcgaaaagaaccccgggaggggagugaaacagaaccugaaaccugaugcauacaaacagugggagcgcccuaguggugugacugcguaccuuuuguauaaugggucaacgacuuacauucaguagcgagcuuaaccggauaggggaggcguagggaaaccgagucuuaauagggcgaugaguugcuggguguagacccgaaaccgagugaucuauccauggccagguugaaggugccguaacagguacuggaggaccgaacccacgcauguugcaaaaugcggggaugagcuguggguaggggugaaaggcuaaacaaacucggagauagcugguucuccccgaaaacuauuuagguagugccucgagcaagacacugauggggguaaagcacuguuauggcuaggggguuauugcaacuuaccaacccauggcaaacucagaauaccaucaagugguuccucgggagacagacagcgggugcuaacguccguugucaagagggaaacaacccagaccgccggcuaaggucccaaaugauagauuaagugguaaacgaagugggaaggcacagacagccaggauguuggcuuagaagcagccaucauuuaaagaaagcguaauagcucacuggucgagucguccugcgcggaagauguaacggggcucaaaucuauaacccaagcugcguaugccgguuuaccggcaugguaggggagcguucuguaggcugaugaaggugcauuguaaagugugcuggagguaucagaagugcgaauguugacaugaguagcgauaaagcgggugaaaagcccgcucgccgcaaagcccaagguuuccuacgcaacguucaucggcguagggugagucggccccuaaggcgaggcagaaaugcguagucgaugggaaacagguuaauauuccuguacuugauucaaaugcgauguggggacggagaagguuagguuggcaagcuguuggaauagcuuguuuaagccgguagguggaagacuuaggcaaauccggguuuucuuaacaccgagaagugaugacgagugucuacggacacgaagcaaccgauaccacgcuuccaggaaaagccacuaagcuucaguuugaaucgaaccguaccgcaaaccgacacaggugggcaggaugagaauucuaaggcgcuugagagaacucgggagaaggaacucggcaaauugauaccguaacuucgggagaagguaugcccucuaagguuaaggacuugcuccguaagccccggagggucgcagagaauagguggcugcgacuguuuauuaaaaacacagcacucugccaacacgaaaguggacguauagggugugacgccugcccggugccggaagguuaauugaagaugugcaagcaucggaucgaagccccgguaaacggcggccguaacuauaacgguccuaagguagcgaaauuccuugucggguaaguuccgacccgcacgaauggcguaacgauggccacacugucuccucccgagacucagcgaaguugaagugguugugaagaugcaaucuacccgcugcuagacggaaagaccccgugaaccuuuacuguagcuuugcauuggacuuugaagucacuuguguaggauaggugggaggcuuggaagcagagacgccagucucuguggagucguccuugaaauaccacccuggugucuuugagguucuaacccagacccgucauccgggucggggaccgugcaugguaggcaguuugacuggggcggucuccucccaaagcguaacggaggaguucgaagguuaccuagguccggucggaaaucggacugauagugcaauggcaaaagguagcuuaacugcgagaccgacaagucgggcaggugcgaaagcaggacauagugauccggugguucuguauggaagggccaucgcucaacggauaaaagguacuccggggauaacaggcugauuccgcccaagaguucauaucgacggcggaguuuggcaccucgaugucggcucaucacauccuggggcuguagucggucccaaggguauggcuguucgccauuuaaagugguacgugagcuggguuuaaaacgucgugagacaguuuggucccuaucugcagugggcguuggaaguuugacgggggcugcuccuaguacgagaggaccggaguggacgaaccucugguguaccgguuguaacgccaguugcauagccggguagcuaaguucggaagagauaagcgcugaaagcaucuaagcgcgaaacucgccugaagaugagacuucccuugcgguuuaaccgcacuaaagggucguucgagaccaggacguugauaggugggguguggaagcgcgguaacgcgugaagcuaacccauacuaauugcccgugaggcuugacucu

SEQ ID NO: 10 - DNA, 1545 bp, Neisseria gonorrhoeae; identical to SEQ ID NO: 4

SEQ ID NO: 11 - RNA, 1545 nt, Neisseria gonorrhoeae; identical to SEQ ID NO: 6

SEQ ID NO: 12 - DNA, 1545 bp, Neisseria gonorrhoeae; identical to SEQ ID NO: 5

SEQ ID NO: 13 - DNA, 1545 bp, Neisseria gonorrhoeae; identical to SEQ ID NO: 4

SEQ ID NO: 14 - DNA, 1545 bp, Neisseria gonorrhoeae; identical to SEQ ID NO: 5

SEQ ID NO: 15 - RNA, 1545 nt, Neisseria gonorrhoeae; identical to SEQ ID NO: 6

SEQ ID NO: 16 - DNA, 2903 bp, Neisseria gonorrhoeae
tgaaatgatagagtcaagtgaataagtgcatcaggcggatgccttggcgatgataggcgacgaaggacgtgtaagcctgcgaaaagcgcgggggagctggcaataaagcaatgatcccgcggtgtccgaatggggaaacccactgcattctgtgcagtatcctaagttgaatacataggcttagagaagcgaacccggagaactgaaccatctaagtacccggaggaaaagaaatcaaccgagattccgcaagtagtggcgagcgaacgcggaggagcctgtacgtaataactgtcgaggtagaagaacaagctgggaagcttgaccatagcgggtgacagtcccgtattcgaaatctcaacagcggtactaagcgtacgaaaagtagggcgggacacgtgaaatcctgtctgaatatggggggaccatcctccaaggctaaatactcatcatcgaccgatagtgaaccagtaccgtgagggaaaggcgaaaagaaccccgggaggggagtgaaacagaacctgaaacctgatgcatacaaacagtgggagcgccctagtggtgtgactgcgtaccttttgtataatgggtcaacgacttacattcagtagcgagcttaaccggataggggaggcgtagggaaaccgagtcttaatagggcgatgagttgctgggtgtagacccgaaaccgagtgatctatccatggccaggttgaaggtgccgtaacaggtactggaggaccgaacccacgcatgttgcaaaatgcggggatgagctgtgggtaggggtgaaaggctaaacaaactcggagatagctggttctccccgaaaactatttaggtagtgcctcgagcaagacactgatgggggtaaagcactgttatggctagggggttattgcaacttaccaacccatggcaaactcagaataccatcaagtggttcctcgggagacagacagcgggtgctaacgtccgttgtcaagagggaaacaacccagaccgccggctaaggtcccaaatgatagattaagtggtaaacgaagtgggaaggcacagacagccaggatgttggcttagaagcagccatcatttaaagaaagcgtaatagctcactggtcgagtcgtcctgcgcggaagatgtaacggggctcaaatctataaccgaagctgcggatgccggtttaccggcatggtaggggagcgttctgtaggctgatgaaggtgcattgtaaagtgtgctggaggtatcagaagtgcgaatgttgacatgagtagcgataaagcgggtgaaaagcccgctcgccgaaagcccaaggtttcctacgcaacgttcatcggcgtagggtgagtcggcccctaaggcgaggcagaaatgcgtagtcgatgggaaacaggttaatattcctgtacttgattcaaatgcgatgtggggacggagaaggttaggttggcaagctgttggaatagcttgtttaagccggtaggtggaagacttaggcaaatccgggttttcttaacaccgagaagtgatgacgagtgtctacggacacgaagcaaccgataccacgcttccaggaaaagccactaagcttcagtttgaatcgaaccgtaccgcaaaccgacacaggtgggcaggatgagaattctaaggcgcttgagagaactcgggagaaggaactcggcaaattgataccgtaacttcgggagaaggtatgccctctaaggttaaggacttgctccgtaagccccggagggtcgcagagaataggtggctgcgactgtttattaaaaacacagcactctgccaacacgaaagtggacgtatagggtgtgacgcctgcccggtgccggaaggttaattgaagatgtgcaagcatcggatcgaagccccggtaaacggcggccgtaactataacggtcctaaggtagcgaaattccttgtcgggtaagttccgacccgcacgaatggcgtaacgatggccacactgtctcctcccgagactcagcgaagttgaagtggttgtgaagatgcaatctacccgctgctagacggaaagaccccgtgaacctttactgtagctttgcattggactttgaagtcacttgtgtaggataggtgggaggcttggaagcagagacgccagtctctgtggagtcgtccttgaaataccaccctggtgtctttgaggttctaacccagacccgtcatccgggtcggggaccgtgcatggtaggcagtttgactggggcggtctcctcccaaagcgtaacggaggagttcgaaggttacctaggtccggtcggaaatcggactgatagtgcaatggcaaaaggtagcttaactgcgagaccgacaagtcgggcaggtgcgaaagcaggacatagtgatccggtggttctgtatggaagggccatcgctcaacggataaaaggtactccggggataacaggctgattccgcccaagagttcatatcgacggcggagtttggcacctcgatgtcggctcatcacatcctggggctgtagtcggtcccaagggtatggctgttcgccatttaaagtggtacgtgagctgggtttaaaacgtcgtgagacagtttggtccctatctgcagtgggcgttggaagtttgacgggggctgctcctagtacgagaggaccggagtggacgaacctctggtgtaccggttgtaacgccagttgcatagccgggtagctaagttcggaagagataagcgctgaaagcatctaagcgcgaaactcgcctgaagatgagacttcccttgcggtttaaccgcactaaagggtcgttcgagaccaggacgttgataggtggggtgtggaagcgcggtaacgcgtgaagctaacccatactaattgcccgtgaggcttgactct

SEQ ID NO: 17 - DNA, 2903 bp, Neisseria gonorrhoeae
agagtcaagcctcacgggcaattagtatgggttagcttcacgcgttaccgcgcttccacaccccacctatcaacgtcctggtctcgaacgaccctttagtgcggttaaaccgcaagggaagtctcatcttcaggcgagtttcgcgcttagatgctttcagcgcttatctcttccgaacttagctacccggctatgcaactggcgttacaaccggtacaccagaggttcgtccactccggtcctctcgtactaggagcagcccccgtcaaacttccaacgcccactgcagatagggaccaaactgtctcacgacgttttaaacccagctcacgtaccactttaaatggcgaacagccatacccttgggaccgactacagccccaggatgtgatgagccgacatcgaggtgccaaactccgccgtcgatatgaactcttgggcggaatcagcctgttatccccggagtaccttttatccgttgagcgatggcccttccatacagaaccaccggatcactatgtcctgctttcgcacctgcccgacttgtcggtctcgcagttaagctaccttttgccattgcactatcagtccgatttccgaccggacctaggtaaccttcgaactcctccgttacgctttgggaggagaccgccccagtcaaactgcctaccatgcacggtccccgacccggatgacgggtctgggttagaacctcaaagacaccagggtggtatttcaaggacgactccacagagactggcgtctctgcttccaagcctcccacctatcctacacaagtgacttcaaagtccaatgcaaagctacagtaaaggttcacggggtctttccgtctagcagcgggtagattgcatcttcacaaccacttcaacttcgctgagtctcgggaggagacagtgtggccatcgttacgccattcgtgcgggtcggaacttacccgacaaggaatttcgctaccttaggaccgttatagttacggccgccgtttaccggggcttcgatccgatgcttgcacatcttcaattaaccttccggcaccgggcaggcgtcacaccctatacgtccactttcgtgttggcagagtgctgtgtttttaataaacagtcgcagccacctattctctgcgaccctccggggcttacggagcaagtccttaaccttagagggcataccttctcccgaagttacggtatcaatttgccgagttccttctcccgagttctctcaagcgccttagaattctcatcctgcccacctgtgtcggtttgcggtacggttcgattcaaactgaagcttagtggcttttcctggaagcgtggtatcggttgcttcgtgtccgtagacactcgtcatcacttctcggtgttaagaaaacccggatttgcctaagtcttccacctaccggcttaaacaagctattccaacagcttgccaacctaaccttctccgtccccacatcgcatttgaatcaagtacaggaatattaacctgtttcccatcgactacgcatttctgcctcgccttaggggccgactcaccctacgccgatgaacgttgcgtaggaaaccttgggctttcggcgagcgggcttttcacccgctttatcgctactcatgtcaacattcgcacttctgatacctccagcacactttacaatgcaccttcatcagcctacagaacgctcccctaccatgccggtaaaccggcatccgcagcttcggttatagatttgagccccgttacatcttccgcgcaggacgactcgaccagtgagctattacgctttctttaaatgatggctgcttctaagccaacatcctggctgtctgtgccttcccacttcgtttaccacttaatctatcatttgggaccttagccggcggtctgggttgtttccctcttgacaacggacgttagcacccgctgtctgtctcccgaggaaccacttgatggtattctgagtttgccatgggttggtaagttgcaataaccccctagccataacagtgctttacccccatcagtgtcttgctcgaggcactacctaaatagttttcggggagaaccagctatctccgagtttgtttagcctttcacccctacccacagctcatccccgcattttgcaacatgcgtgggttcggtcctccagtacctgttacggcaccttcaacctggccatggatagatcactcggtttcgggtctacacccagcaactcatcgccctattaagactcggtttccctacgcctcccctatccggttaagctcgctactgaatgtaagtcgttgacccattatacaaaaggtacgcagtcacaccactagggcgctcccactgtttgtatgcatcaggtttcaggttctgtttcactcccctcccggggttcttttcgcctttccctcacggtactggttcactatcggtcgatgatgagtatttagccttggaggatggtccccccatattcagacaggatttcacgtgtcccgccctacttttcgtacgcttagtaccgctgttgagatttcgaatacgggactgtcacccgctatggtcaagcttcccagcttgttcttctacctcgacagttattacgtacaggctcctccgcgttcgctcgccactacttgcggaatctcggttgatttcttttcctccgggtacttagatggttcagttctccgggttcgcttctctaagcctatgtattcaacttaggatactgcacagaatgcagtgggtttccccattcggacaccgcgggatcattgctttattgccagctcccccgcgcttttcgcaggcttacacgtccttcgtcgcctatcatcgccaaggcatccgcctgatgcacttattcacttgactctatcatttca

SEQ ID NO: 18 - RNA, 2903 nt, Neisseria gonorrhoeae
ugaaaugauagagucaagugaauaagugcaucaggcggaugccuuggcgaugauaggcgacgaaggacguguaagccugcgaaaagcgcgggggagcuggcaauaaagcaaugaucccgcgguguccgaauggggaaacccacugcauucugugcaguauccuaaguugaauacauaggcuuagagaagcgaacccggagaacugaaccaucuaaguacccggaggaaaagaaaucaaccgagauuccgcaaguaguggcgagcgaacgcggaggagccuguacguaauaacugucgagguagaagaacaagcugggaagcuugaccauagcgggugacagucccguauucgaaaucucaacagcgguacuaagcguacgaaaaguagggcgggacacgugaaauccugucugaauauggggggaccauccuccaaggcuaaauacucaucaucgaccgauagugaaccaguaccgugagggaaaggcgaaaagaaccccgggaggggagugaaacagaaccugaaaccugaugcauacaaacagugggagcgcccuaguggugugacugcguaccuuuuguauaaugggucaacgacuuacauucaguagcgagcuuaaccggauaggggaggcguagggaaaccgagucuuaauagggcgaugaguugcuggguguagacccgaaaccgagugaucuauccauggccagguugaaggugccguaacagguacuggaggaccgaacccacgcauguugcaaaaugcggggaugagcuguggguaggggugaaaggcuaaacaaacucggagauagcugguucuccccgaaaacuauuuagguagugccucgagcaagacacugauggggguaaagcacuguuauggcuaggggguuauugcaacuuaccaacccauggcaaacucagaauaccaucaagugguuccucgggagacagacagcgggugcuaacguccguugucaagagggaaacaacccagaccgccggcuaaggucccaaaugauagauuaagugguaaacgaagugggaaggcacagacagccaggauguuggcuuagaagcagccaucauuuaaagaaagcguaauagcucacuggucgagucguccugcgcggaagauguaacggggcucaaaucuauaaccgaagcugcggaugccgguuuaccggcaugguaggggagcguucuguaggcugaugaaggugcauuguaaagugugcuggagguaucagaagugcgaauguugacaugaguagcgauaaagcgggugaaaagcccgcucgccgaaagcccaagguuuccuacgcaacguucaucggcguagggugagucggccccuaaggcgaggcagaaaugcguagucgaugggaaacagguuaauauuccuguacuugauucaaaugcgauguggggacggagaagguuagguuggcaagcuguuggaauagcuuguuuaagccgguagguggaagacuuaggcaaauccggguuuucuuaacaccgagaagugaugacgagugucuacggacacgaagcaaccgauaccacgcuuccaggaaaagccacuaagcuucaguuugaaucgaaccguaccgcaaaccgacacaggugggcaggaugagaauucuaaggcgcuugagagaacucgggagaaggaacucggcaaauugauaccguaacuucgggagaagguaugcccucuaagguuaaggacuugcuccguaagccccggagggucgcagagaauagguggcugcgacuguuuauuaaaaacacagcacucugccaacacgaaaguggacguauagggugugacgccugcccggugccggaagguuaauugaagaugugcaagcaucggaucgaagccccgguaaacggcggccguaacuauaacgguccuaagguagcgaaauuccuugucggguaaguuccgacccgcacgaauggcguaacgauggccacacugucuccucccgagacucagcgaaguugaagugguugugaagaugcaaucuacccgcugcuagacggaaagaccccgugaaccuuuacuguagcuuugcauuggacuuugaagucacuuguguaggauaggugggaggcuuggaagcagagacgccagucucuguggagucguccuugaaauaccacccuggugucuuugagguucuaacccagacccgucauccgggucggggaccgugcaugguaggcaguuugacuggggcggucuccucccaaagcguaacggaggaguucgaagguuaccuagguccggucggaaaucggacugauagugcaauggcaaaagguagcuuaacugcgagaccgacaagucgggcaggugcgaaagcaggacauagugauccggugguucuguauggaagggccaucgcucaacggauaaaagguacuccggggauaacaggcugauuccgcccaagaguucauaucgacggcggaguuuggcaccucgaugucggcucaucacauccuggggcuguagucggucccaaggguauggcuguucgccauuuaaagugguacgugagcuggguuuaaaacgucgugagacaguuuggucccuaucugcagugggcguuggaaguuugacgggggcugcuccuaguacgagaggaccggaguggacgaaccucugguguaccgguuguaacgccaguugcauagccggguagcuaaguucggaagagauaagcgcugaaagcaucuaagcgcgaaacucgccugaagaugagacuucccuugcgguuuaaccgcacuaaagggucguucgagaccaggacguugauaggugggguguggaagcgcgguaacgcgugaagcuaacccauacuaauugcccgugaggcuugacucu

SEQ ID NO: 19 - DNA, 1545 bp, Neisseria gonorrhoeae; identical to SEQ ID NO: 4

SEQ ID NO: 20 - DNA, 1545 bp, Neisseria gonorrhoeae; identical to SEQ ID NO: 5

SEQ ID NO: 21 - RNA, 1545 nt, Neisseria gonorrhoeae; identical to SEQ ID NO: 6

SEQ ID NO: 22 - DNA, 2903 bp, Neisseria gonorrhoeae
tgaaatgatagagtcaagtgaataagtgcatcaggcggatgccttggcgatgataggcgacgaaggacgtgtaagcctgcgaaaagcgcgggggagctggcaataaagcaatgatcccgcggtgtccgaatggggaaacccactgcattctgtgcagtatcctaagttgaatacataggcttagagaagcgaacccggagaactgacccatctaagtacccggaggaaaagaaatcaaccgagattccgcaagtagtggcgagcgaacgcggaggagcctgtacgtaataactgtcgaggtagaagaacaagctgggaagcttgaccatagcgggtgacagtcccgtattcgaaatctcaacagcggtactaagcgtacgaaaagtagggcgggacacgtgaaatcctgtctgaatatggggggaccatcctccaaggctaaatactcatcatcgaccgatagtgaaccagtaccgtgagggaaaggcgaaaagaaccccgggaggggagtgaaacagaacctgaaacctgatgcatacaaacagtgggagcgccctagtggtgtgactgcgtaccttttgtataatgggtcaacgacttacattcagtagcgagcttaaccggataggggaggcgtagggaaaccgagtcttaatagggcgatgagttgctgggtgtagacccgaaaccgagtgatctatccatggccaggttgaaggtgccgtaacaggtactggaggaccgaacccacgcatgttgcaaaatgcggggatgagctgtgggtaggggtgaaaggctaaacaaactcggagatagctggttctccccgaaaactatttaggtagtgcctcgagcaagacactgatgggggtaaagcactgttatggctagggggttattgcaacttaccaacccatggcaaactcagaataccatcaagtggttcctcgggagacagacagcgggtgctaacgtccgttgtcaagagggaaacaacccagaccgccggctaaggtcccaaatgatagattaagtggtaaacgaagtgggaaggcacagacagccaggatgttggcttagaagcagccatcatttaaagaaagcgtaatagctcactggtcgagtcgtcctgcgcggaagatgtaacggggctcaaatctataaccgaagctgcggatgccggtttaccggcatggtaggggagcgttctgtaggctgatgaaggtgcattgtaaagtgtgctggaggtatcagaagtgcgaatgttgacatgagtagcgataaagcgggtgaaaagcccgctcgccgaaagcccaaggtttcctacgcaacgttcatcggcgtagggtgagtcggcccctaaggcgaggcagaaatgcgtagtcgatgggaaacaggttaatattcctgtacttgattcaaatgcgatgtggggacggagaaggttaggttggcaagctgttggaatagcttgtttaagccggtaggtggaagacttaggcaaatccgggttttcttaacaccgagaagtgatgacgagtgtctacggacacgaagcaaccgataccacgcttccaggaaaagccactaagcttcagtttgaatcgaaccgtaccgcaaaccgacacaggtgggcaggatgagaattctaaggcgcttgagagaactcgggagaaggaactcggcaaattgataccgtaacttcgggagaaggtatgccctctaaggttaaggacttgctccgtaagccccggagggtcgcagagaataggtggctgcgactgtttattaaaaacacagcactctgccaacacgaaagtggacgtatagggtgtgacgcctgcccggtgccggaaggttaattgaagatgtgcaagcatcggatcgaagccccggtaaacggcggccgtaactataacggtcctaaggtagcgaaattccttgtcgggtaagttccgacccgcacgaatggcgtaacgatggccacactgtctcctcccgagactcagcgaagttgaagtggttgtgaagatgcaatctacccgctgctagacggaaagaccccgtgaacctttactgtagctttgcattggactttgaagtcacttgtgtaggataggtgggaggcttggaagcagagacgccagtctctgtggagtcgtccttgaaataccaccctggtgtctttgaggttctaacccagacccgtcatccgggtcggggaccgtgcatggtaggcagtttgactggggcggtctcctcccaaagcgtaacggaggagttcgaaggttacctaggtccggtcggaaatcggactgatagtgcaatggcaaaaggtagcttaactgcgagaccgacaagtcgggcaggtgcgaaagcaggacatagtgatccggtggttctgtatggaagggccatcgctcaacggataaaaggtactccggggataacaggctgattccgcccaagagttcatatcgacggcggagtttggcacctcgatgtcggctcatcacatcctggggctgtagtcggtcccaagggtatggctgttcgccatttaaagtggtacgtgagctgggtttaaaacgtcgtgagacagtttggtccctatctgcagtgggcgttggaagtttgacgggggctgctcctagtacgagaggaccggagtggacgaacctctggtgtaccggttgtaacgccagttgcatagccgggtagctaagttcggaagagataagcgctgaaagcatctaagcgcgaaactcgcctgaagatgagacttcccttgcggtttaaccgcactaaagggtcgttcgagaccaggacgttgataggtggggtgtggaagcgcggtaacgcgtgaagctaacccatactaattgcccgtgaggcttgactct

SEQ ID NO: 23 - DNA, 2903 bp, Neisseria gonorrhoeae
agagtcaagcctcacgggcaattagtatgggttagcttcacgcgttaccgcgcttccacaccccacctatcaacgtcctggtctcgaacgaccctttagtgcggttaaaccgcaagggaagtctcatcttcaggcgagtttcgcgcttagatgctttcagcgcttatctcttccgaacttagctacccggctatgcaactggcgttacaaccggtacaccagaggttcgtccactccggtcctctcgtactaggagcagcccccgtcaaacttccaacgcccactgcagatagggaccaaactgtctcacgacgttttaaacccagctcacgtaccactttaaatggcgaacagccatacccttgggaccgactacagccccaggatgtgatgagccgacatcgaggtgccaaactccgccgtcgatatgaactcttgggcggaatcagcctgttatccccggagtaccttttatccgttgagcgatggcccttccatacagaaccaccggatcactatgtcctgctttcgcacctgcccgacttgtcggtctcgcagttaagctaccttttgccattgcactatcagtccgatttccgaccggacctaggtaaccttcgaactcctccgttacgctttgggaggagaccgccccagtcaaactgcctaccatgcacggtccccgacccggatgacgggtctgggttagaacctcaaagacaccagggtggtatttcaaggacgactccacagagactggcgtctctgcttccaagcctcccacctatcctacacaagtgacttcaaagtccaatgcaaagctacagtaaaggttcacggggtctttccgtctagcagcgggtagattgcatcttcacaaccacttcaacttcgctgagtctcgggaggagacagtgtggccatcgttacgccattcgtgcgggtcggaacttacccgacaaggaatttcgctaccttaggaccgttatagttacggccgccgtttaccggggcttcgatccgatgcttgcacatcttcaattaaccttccggcaccgggcaggcgtcacaccctatacgtccactttcgtgttggcagagtgctgtgtttttaataaacagtcgcagccacctattctctgcgaccctccggggcttacggagcaagtccttaaccttagagggcataccttctcccgaagttacggtatcaatttgccgagttccttctcccgagttctctcaagcgccttagaattctcatcctgcccacctgtgtcggtttgcggtacggttcgattcaaactgaagcttagtggcttttcctggaagcgtggtatcggttgcttcgtgtccgtagacactcgtcatcacttctcggtgttaagaaaacccggatttgcctaagtcttccacctaccggcttaaacaagctattccaacagcttgccaacctaaccttctccgtccccacatcgcatttgaatcaagtacaggaatattaacctgtttcccatcgactacgcatttctgcctcgccttaggggccgactcaccctacgccgatgaacgttgcgtaggaaaccttgggctttcggcgagcgggcttttcacccgctttatcgctactcatgtcaacattcgcacttctgatacctccagcacactttacaatgcaccttcatcagcctacagaacgctcccctaccatgccggtaaaccggcatccgcagcttcggttatagatttgagccccgttacatcttccgcgcaggacgactcgaccagtgagctattacgctttctttaaatgatggctgcttctaagccaacatcctggctgtctgtgccttcccacttcgtttaccacttaatctatcatttgggaccttagccggcggtctgggttgtttccctcttgacaacggacgttagcacccgctgtctgtctcccgaggaaccacttgatggtattctgagtttgccatgggttggtaagttgcaataaccccctagccataacagtgctttacccccatcagtgtcttgctcgaggcactacctaaatagttttcggggagaaccagctatctccgagtttgtttagcctttcacccctacccacagctcatccccgcattttgcaacatgcgtgggttcggtcctccagtacctgttacggcaccttcaacctggccatggatagatcactcggtttcgggtctacacccagcaactcatcgccctattaagactcggtttccctacgcctcccctatccggttaagctcgctactgaatgtaagtcgttgacccattatacaaaaggtacgcagtcacaccactagggcgctcccactgtttgtatgcatcaggtttcaggttctgtttcactcccctcccggggttcttttcgcctttccctcacggtactggttcactatcggtcgatgatgagtatttagccttggaggatggtccccccatattcagacaggatttcacgtgtcccgccctacttttcgtacgcttagtaccgctgttgagatttcgaatacgggactgtcacccgctatggtcaagcttcccagcttgttcttctacctcgacagttattacgtacaggctcctccgcgttcgctcgccactacttgcggaatctcggttgatttcttttcctccgggtacttagatgggtcagttctccgggttcgcttctctaagcctatgtattcaacttaggatactgcacagaatgcagtgggtttccccattcggacaccgcgggatcattgctttattgccagctcccccgcgcttttcgcaggcttacacgtccttcgtcgcctatcatcgccaaggcatccgcctgatgcacttattcacttgactctatcatttca

SEQ ID NO: 24 - RNA, 2903 nt, Neisseria gonorrhoeae
ugaaaugauagagucaagugaauaagugcaucaggcggaugccuuggcgaugauaggcgacgaaggacguguaagccugcgaaaagcgcgggggagcuggcaauaaagcaaugaucccgcgguguccgaauggggaaacccacugcauucugugcaguauccuaaguugaauacauaggcuuagagaagcgaacccggagaacugacccaucuaaguacccggaggaaaagaaaucaaccgagauuccgcaaguaguggcgagcgaacgcggaggagccuguacguaauaacugucgagguagaagaacaagcugggaagcuugaccauagcgggugacagucccguauucgaaaucucaacagcgguacuaagcguacgaaaaguagggcgggacacgugaaauccugucugaauauggggggaccauccuccaaggcuaaauacucaucaucgaccgauagugaaccaguaccgugagggaaaggcgaaaagaaccccgggaggggagugaaacagaaccugaaaccugaugcauacaaacagugggagcgcccuaguggugugacugcguaccuuuuguauaaugggucaacgacuuacauucaguagcgagcuuaaccggauaggggaggcguagggaaaccgagucuuaauagggcgaugaguugcuggguguagacccgaaaccgagugaucuauccauggccagguugaaggugccguaacagguacuggaggaccgaacccacgcauguugcaaaaugcggggaugagcuguggguaggggugaaaggcuaaacaaacucggagauagcugguucuccccgaaaacuauuuagguagugccucgagcaagacacugauggggguaaagcacuguuauggcuaggggguuauugcaacuuaccaacccauggcaaacucagaauaccaucaagugguuccucgggagacagacagcgggugcuaacguccguugucaagagggaaacaacccagaccgccggcuaaggucccaaaugauagauuaagugguaaacgaagugggaaggcacagacagccaggauguuggcuuagaagcagccaucauuuaaagaaagcguaauagcucacuggucgagucguccugcgcggaagauguaacggggcucaaaucuauaaccgaagcugcggaugccgguuuaccggcaugguaggggagcguucuguaggcugaugaaggugcauuguaaagugugcuggagguaucagaagugcgaauguugacaugaguagcgauaaagcgggugaaaagcccgcucgccgaaagcccaagguuuccuacgcaacguucaucggcguagggugagucggccccuaaggcgaggcagaaaugcguagucgaugggaaacagguuaauauuccuguacuugauucaaaugcgauguggggacggagaagguuagguuggcaagcuguuggaauagcuuguuuaagccgguagguggaagacuuaggcaaauccggguuuucuuaacaccgagaagugaugacgagugucuacggacacgaagcaaccgauaccacgcuuccaggaaaagccacuaagcuucaguuugaaucgaaccguaccgcaaaccgacacaggugggcaggaugagaauucuaaggcgcuugagagaacucgggagaaggaacucggcaaauugauaccguaacuucgggagaagguaugcccucuaagguuaaggacuugcuccguaagccccggagggucgcagagaauagguggcugcgacuguuuauuaaaaacacagcacucugccaacacgaaaguggacguauagggugugacgccugcccggugccggaagguuaauugaagaugugcaagcaucggaucgaagccccgguaaacggcggccguaacuauaacgguccuaagguagcgaaauuccuugucggguaaguuccgacccgcacgaauggcguaacgauggccacacugucuccucccgagacucagcgaaguugaagugguugugaagaugcaaucuacccgcugcuagacggaaagaccccgugaaccuuuacuguagcuuugcauuggacuuugaagucacuuguguaggauaggugggaggcuuggaagcagagacgccagucucuguggagucguccuugaaauaccacccuggugucuuugagguucuaacccagacccgucauccgggucggggaccgugcaugguaggcaguuugacuggggcggucuccucccaaagcguaacggaggaguucgaagguuaccuagguccggucggaaaucggacugauagugcaauggcaaaagguagcuuaacugcgagaccgacaagucgggcaggugcgaaagcaggacauagugauccggugguucuguauggaagggccaucgcucaacggauaaaagguacuccggggauaacaggcugauuccgcccaagaguucauaucgacggcggaguuuggcaccucgaugucggcucaucacauccuggggcuguagucggucccaaggguauggcuguucgccauuuaaagugguacgugagcuggguuuaaaacgucgugagacaguuuggucccuaucugcagugggcguuggaaguuugacgggggcugcuccuaguacgagaggaccggaguggacgaaccucugguguaccgguuguaacgccaguugcauagccggguagcuaaguucggaagagauaagcgcugaaagcaucuaagcgcgaaacucgccugaagaugagacuucccuugcgguuuaaccgcacuaaagggucguucgagaccaggacguugauaggugggguguggaagcgcgguaacgcgugaagcuaacccauacuaauugcccgugaggcuugacucu

SEQ ID NO: 25 - DNA, 1545 bp, Neisseria gonorrhoeae; identical to SEQ ID NO: 4

SEQ ID NO: 26 - DNA, 1545 bp, Neisseria gonorrhoeae; identical to SEQ ID NO: 5

SEQ ID NO: 27 - RNA, 1545 nt, Neisseria gonorrhoeae; identical to SEQ ID NO: 6

SEQ ID NO: 28 - DNA, 1047 bp, Neisseria gonorrhoeae
atgaaaaaatccctgattgccctgactttggcagcccttcctgttgcggcaatggccgatgtcaccctgtacggcgccatcaaagccggcgtacaaacttaccgttctgtagaacatacagacggcaaggtaagtaaagtggaaaccggcagcgaaatcgccgacttcggttcaaaaatcggcttcaaaggccaagaagacctcggcaacggcctgaaggccgtttggcagttggaacaaggtgcctccgtcgccggcactaacaccggctggggcaacaaacaatccttcgtcggcttgaagggcggcttcggtaccatccgcgccggtagcctgaacagccccctgaaaaacaccggcgccaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcaggctcaaacggcgaatcttaccacgttggcttgaactaccaaaacagcggcttcttcgcgcaatacgccggcttgttccaaagatacggcgaaggcactaaaaaaatcgaatacgatggtcaaacttatagtatccccagtctgtttgttgaaaaactgcaagttcaccgtttggtaggcggttacgacaataatgccctgtacgtttccgtagccgcacaacaacaagatgccaaattgtatggagcaatgagcggtaattcgcacaactctcaaaccgaagttgccgctaccgcggcataccgtttcggcaatgtaacgccccgcgtttcttacgcccacggcttcaaaggcactgttgatagtgcaaaccacgacaatacttatgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 29 - DNA, 1047 bp, Neisseria gonorrhoeae
ttagaatttgtggcgcagaacgacggcgctggcagtcgatacgattttgtctgcgcctttgccttcttgcaaccagccggcagaaaccaaggcagaagtgcgtttggagaagtcgtattccgcaccgacaaccacttggtcataagtattgtcgtggtttgcactatcaacagtgcctttgaagccgtgggcgtaagaaacgcggggcgttacattgccgaaacggtatgccgcggtagcggcaacttcggtttgagagttgtgcgaattaccgctcattgctccatacaatttggcatcttgttgttgtgcggctacggaaacgtacagggcattattgtcgtaaccgcctaccaaacggtgaacttgcagtttttcaacaaacagactggggatactataagtttgaccatcgtattcgatttttttagtgccttcgccgtatctttggaacaagccggcgtattgcgcgaagaagccgctgttttggtagttcaagccaacgtggtaagattcgccgtttgagcctgaattgtctttaggtgcgtattgtacgctgccgctgaagccggcaaattcgggagaatcgtagcgtacggacaggtagcggtgttcccgttgggccattccgctgatttccagcacattgccggtaaatttgccggattcccaagcattgacgttggcgccggtgtttttcagggggctgttcaggctaccggcgcggatggtaccgaagccgcccttcaagccgacgaaggattgtttgttgccccagccggtgttagtgccggcgacggaggcaccttgttccaactgccaaacggccttcaggccgttgccgaggtcttcttggcctttgaagccgatttttgaaccgaagtcggcgatttcgctgccggtttccactttacttaccttgccgtctgtatgttctacagaacggtaagtttgtacgccggctttgatggcgccgtacagggtgacatcggccattgccgcaacaggaagggctgccaaagtcagggcaatcagggattttttcat

SEQ ID NO: 30 - RNA, 1047 nt, Neisseria gonorrhoeae
augaaaaaaucccugauugcccugacuuuggcagcccuuccuguugcggcaauggccgaugucacccuguacggcgccaucaaagccggcguacaaacuuaccguucuguagaacauacagacggcaagguaaguaaaguggaaaccggcagcgaaaucgccgacuucgguucaaaaaucggcuucaaaggccaagaagaccucggcaacggccugaaggccguuuggcaguuggaacaaggugccuccgucgccggcacuaacaccggcuggggcaacaaacaauccuucgucggcuugaagggcggcuucgguaccauccgcgccgguagccugaacagcccccugaaaaacaccggcgccaacgucaaugcuugggaauccggcaaauuuaccggcaaugugcuggaaaucagcggaauggcccaacgggaacaccgcuaccuguccguacgcuacgauucucccgaauuugccggcuucagcggcagcguacaauacgcaccuaaagacaauucaggcucaaacggcgaaucuuaccacguuggcuugaacuaccaaaacagcggcuucuucgcgcaauacgccggcuuguuccaaagauacggcgaaggcacuaaaaaaaucgaauacgauggucaaacuuauaguauccccagucuguuuguugaaaaacugcaaguucaccguuugguaggcgguuacgacaauaaugcccuguacguuuccguagccgcacaacaacaagaugccaaauuguauggagcaaugagcgguaauucgcacaacucucaaaccgaaguugccgcuaccgcggcauaccguuucggcaauguaacgccccgcguuucuuacgcccacggcuucaaaggcacuguugauagugcaaaccacgacaauacuuaugaccaagugguugucggugcggaauacgacuucuccaaacgcacuucugccuugguuucugccggcugguugcaagaaggcaaaggcgcagacaaaaucguaucgacugccagcgccgucguucugcgccacaaauucuaa

SEQ ID NO: 31 - DNA, 234 bp, Neisseria gonorrhoeae
atggcacgagtttgcaaagtgaccggtaaacgcccgatgtccggcaacaacgtatcgcacgccaacaacaaaaccaaacgccgttttttgcccaacttgcaatcacgtcgtttttgggtagaaagtgaaaaccgctgggttcgcctgcgcgtttccaacgctgcattgcgtaccatcgacaaagtaggcattgatgtcgtattggctgatttgcgtgctcgcggcgaagcttaa

SEQ ID NO: 32 - DNA, 234 bp, Neisseria gonorrhoeae
ttaagcttcgccgcgagcacgcaaatcagccaatacgacatcaatgcctactttgtcgatggtacgcaatgcagcgttggaaacgcgcaggcgaacccagcggttttcactttctacccaaaaacgacgtgattgcaagttgggcaaaaaacggcgtttggttttgttgttggcgtgcgatacgttgttgccggacatcgggcgtttaccggtcactttgcaaactcgtgccat

SEQ ID NO: 33 - RNA, 234 nt, Neisseria gonorrhoeae
auggcacgaguuugcaaagugaccgguaaacgcccgauguccggcaacaacguaucgcacgccaacaacaaaaccaaacgccguuuuuugcccaacuugcaaucacgucguuuuuggguagaaagugaaaaccgcuggguucgccugcgcguuuccaacgcugcauugcguaccaucgacaaaguaggcauugaugucguauuggcugauuugcgugcucgcggcgaagcuuaa

SEQ ID NO: 34 - DNA, 351 bp, Neisseria gonorrhoeae
atggaagccttcaaaaccctaatttggattattaatattatttccgctttggccgtcatcgtgttagtattgctccaacacggcaaaggcgcggatgccggcgcgaccttcggatcgggaagcggcagcgcgcaaggcgtattcggctctgccggcaacgccaacttcctcagccgctcgaccgccgttgcagcaacatttttctttgcaacctgcatggctatggtgtatattcacacccacacgacaaaacacggtttggacttcagcaacatacgacagactcagcaagcacccaaacccgtaagcaataccgaaccttctgcccctgttcctcagcagcagaaataa

SEQ ID NO: 35 - DNA, 351 bp, Neisseria gonorrhoeae
ttatttctgctgctgaggaacaggggcagaaggttcggtattgcttacgggtttgggtgcttgctgagtctgtcgtatgttgctgaagtccaaaccgtgttttgtcgtgtgggtgtgaatatacaccatagccatgcaggttgcaaagaaaaatgttgctgcaacggcggtcgagcggctgaggaagttggcgttgccggcagagccgaatacgccttgcgcgctgccgcttcccgatccgaaggtcgcgccggcatccgcgcctttgccgtgttggagcaatactaacacgatgacggccaaagcggaaataatattaataatccaaattagggttttgaaggcttccat

SEQ ID NO: 36 - RNA, 351 nt, Neisseria gonorrhoeae
auggaagccuucaaaacccuaauuuggauuauuaauauuauuuccgcuuuggccgucaucguguuaguauugcuccaacacggcaaaggcgcggaugccggcgcgaccuucggaucgggaagcggcagcgcgcaaggcguauucggcucugccggcaacgccaacuuccucagccgcucgaccgccguugcagcaacauuuuucuuugcaaccugcauggcuaugguguauauucacacccacacgacaaaacacgguuuggacuucagcaacauacgacagacucagcaagcacccaaacccguaagcaauaccgaaccuucugccccuguuccucagcagcagaaauaa

SEQ ID NO: 37 - DNA, 366 bp, Neisseria gonorrhoeae
atgaacctgattcaacagctcgagcaagaagaaattgcccgcctgaacaaagaaatccccgaattcgcaccgggcgacaccgtagtcgtatccgtacgcgtcgtggaaggtacccgcagccgtctgcaagcctacgaaggcgtggttatcgcccgtcgcaaccgtggtttgaacagcaacttcatcgtccgcaaaatctccagcggcgaaggtgttgaacgtactttccaactgtattcccctactgttgagaaaatcgaagtcaaacgccgtggcgacgtacgccgtgccaaactgtactacctgcgcggtctgaccggcaaagctgcacgcatcaaagaaaaactgcctgcacgcaaaggttga

SEQ ID NO: 38 - DNA, 366 bp, Neisseria gonorrhoeae
tcaacctttgcgtgcaggcagtttttctttgatgcgtgcagctttgccggtcagaccgcgcaggtagtacagtttggcacggcgtacgtcgccacggcgtttgacttcgattttctcaacagtaggggaatacagttggaaagtacgttcaacaccttcgccgctggagattttgcggacgatgaagttgctgttcaaaccacggttgcgacgggcgataaccacgccttcgtaggcttgcagacggctgcgggtaccttccacgacgcgtacggatacgactacggtgtcgcccggtgcgaattcggggatttctttgttcaggcgggcaatttcttcttgctcgagctgttgaatcaggttcat

SEQ ID NO: 39 - RNA, 366 nt, Neisseria gonorrhoeae
augaaccugauucaacagcucgagcaagaagaaauugcccgccugaacaaagaaauccccgaauucgcaccgggcgacaccguagucguauccguacgcgucguggaagguacccgcagccgucugcaagccuacgaaggcgugguuaucgcccgucgcaaccgugguuugaacagcaacuucaucguccgcaaaaucuccagcggcgaagguguugaacguacuuuccaacuguauuccccuacuguugagaaaaucgaagucaaacgccguggcgacguacgccgugccaaacuguacuaccugcgcggucugaccggcaaagcugcacgcaucaaagaaaaacugccugcacgcaaagguuga

SEQ ID NO: 40 - DNA, 750 bp, Neisseria gonorrhoeae
atgcttatccaggcagttaccattttccccgaaatgttcgacagcattacccgctacggcgtaacgggacgcgcgaacagacagggaatctggcagtttgaagcagtcaatccccgaaagtttgccgacaacagattgggctatatcgacgaccgcccgttcggcggcggtccgggaatgattatgatggctccgccgcttcatgcggcgatagaacacgccaaagcacaatcttcccaaaccgcaaaagtcatctacctcagcccccaaggaaaaccgctgacacaccaaaaagcggcagaactggcagaacttacgcatctgattctgctgtgcggacgctatgagggaatagacgaaagactgctgcaaagcagcgtcgatgaagaaatcagcatcggagacttcgtcgtttccggcggagagcttcccgccatgatgctgatggatgcggtattgaggctcgtacccggcatattgggcgacattcagtctgccgaacaggattcgttctcaagcggtattttggactgcccccactacaccaaacccttagaatttcaaggcatggctgttccggaagtattgcgctccggaaatcatggcttgatagcggaatggcggttggaacaatcgctgcgccgcaccttggagcgcagacccgatcttttggaaaagcgcgttttaatcccaaaggaatcccgcctcttgaataaaatcctacaagagcaacgggaaatccaatcataa

SEQ ID NO: 41 - DNA, 750 bp, Neisseria gonorrhoeae
ttatgattggatttcccgttgctcttgtaggattttattcaagaggcgggattcctttgggattaaaacgcgcttttccaaaagatcgggtctgcgctccaaggtgcggcgcagcgattgttccaaccgccattccgctatcaagccatgatttccggagcgcaatacttccggaacagccatgccttgaaattctaagggtttggtgtagtgggggcagtccaaaataccgcttgagaacgaatcctgttcggcagactgaatgtcgcccaatatgccgggtacgagcctcaataccgcatccatcagcatcatggcgggaagctctccgccggaaacgacgaagtctccgatgctgatttcttcatcgacgctgctttgcagcagtctttcgtctattccctcatagcgtccgcacagcagaatcagatgcgtaagttctgccagttctgccgctttttggtgtgtcagcggttttccttgggggctgaggtagatgacttttgcggtttgggaagattgtgctttggcgtgttctatcgccgcatgaagcggcggagccatcataatcattcccggaccgccgccgaacgggcggtcgtcgatatagcccaatctgttgtcggcaaactttcggggattgactgcttcaaactgccagattccctgtctgttcgcgcgtcccgttacgccgtagcgggtaatgctgtcgaacatttcggggaaaatggtaactgcctggataagcat

SEQ ID NO: 42 - RNA, 750 nt, Neisseria gonorrhoeae
augcuuauccaggcaguuaccauuuuccccgaaauguucgacagcauuacccgcuacggcguaacgggacgcgcgaacagacagggaaucuggcaguuugaagcagucaauccccgaaaguuugccgacaacagauugggcuauaucgacgaccgcccguucggcggcgguccgggaaugauuaugauggcuccgccgcuucaugcggcgauagaacacgccaaagcacaaucuucccaaaccgcaaaagucaucuaccucagcccccaaggaaaaccgcugacacaccaaaaagcggcagaacuggcagaacuuacgcaucugauucugcugugcggacgcuaugagggaauagacgaaagacugcugcaaagcagcgucgaugaagaaaucagcaucggagacuucgucguuuccggcggagagcuucccgccaugaugcugauggaugcgguauugaggcucguacccggcauauugggcgacauucagucugccgaacaggauucguucucaagcgguauuuuggacugcccccacuacaccaaacccuuagaauuucaaggcauggcuguuccggaaguauugcgcuccggaaaucauggcuugauagcggaauggcgguuggaacaaucgcugcgccgcaccuuggagcgcagacccgaucuuuuggaaaagcgcguuuuaaucccaaaggaaucccgccucuugaauaaaauccuacaagagcaacgggaaauccaaucauaa

SEQ ID NO: 43 - DNA, 510 bp, Neisseria gonorrhoeae
atgacagacactcaaaaccgggtagccatgggctacatcaaaggcgtattcggcataaaaggctggctgaaaattgccgccaacaccgaatattccgacagccttttggactaccccgagtggcatttggccaaggacggcaaaaccgtcagcgttacccttgaagccggaaaagtcgtcaacggcgaactccaagtcaaattcgaaggcatagacgaccgcgattcagcattctcattgcgcggttacaccatcgaaataccccgtgaagcattcgccccgacagaagaagacgaatactactgggcagacttggtcggcatgaccgtcgtcaacaaagacgataccgttttaggcaaggtaagcaacctgatggaaaccggcgcaaacgacgtattgatgattgacggagaacacgggcagattctgattccgttcgtttcccaatatatcgaaaccgtcgataccggcagcaagaccattactgccgactggggtttggactactga

SEQ ID NO: 44 - DNA, 510 bp, Neisseria gonorrhoeae
tcagtagtccaaaccccagtcggcagtaatggtcttgctgccggtatcgacggtttcgatatattgggaaacgaacggaatcagaatctgcccgtgttctccgtcaatcatcaatacgtcgtttgcgccggtttccatcaggttgcttaccttgcctaaaacggtatcgtctttgttgacgacggtcatgccgaccaagtctgcccagtagtattcgtcttcttctgtcggggcgaatgcttcacggggtatttcgatggtgtaaccgcgcaatgagaatgctgaatcgcggtcgtctatgccttcgaatttgacttggagttcgccgttgacgacttttccggcttcaagggtaacgctgacggttttgccgtccttggccaaatgccactcggggtagtccaaaaggctgtcggaatattcggtgttggcggcaattttcagccagccttttatgccgaatacgcctttgatgtagcccatggctacccggttttgagtgtctgtcat

SEQ ID NO: 45 - RNA, 510 nt, Neisseria gonorrhoeae
augacagacacucaaaaccggguagccaugggcuacaucaaaggcguauucggcauaaaaggcuggcugaaaauugccgccaacaccgaauauuccgacagccuuuuggacuaccccgaguggcauuuggccaaggacggcaaaaccgucagcguuacccuugaagccggaaaagucgucaacggcgaacuccaagucaaauucgaaggcauagacgaccgcgauucagcauucucauugcgcgguuacaccaucgaaauaccccgugaagcauucgccccgacagaagaagacgaauacuacugggcagacuuggucggcaugaccgucgucaacaaagacgauaccguuuuaggcaagguaagcaaccugauggaaaccggcgcaaacgacguauugaugauugacggagaacacgggcagauucugauuccguucguuucccaauauaucgaaaccgucgauaccggcagcaagaccauuacugccgacugggguuuggacuacuga

SEQ ID NO: 46 - DNA, 246 bp, Neisseria gonorrhoeae
atggtagttatccgtttggcacgcggcggctcgaaacaccgccccttctacaacgtcatcgttactgactcacgcagccgccgcgacggccgcttcatcgaacgcgtaggcttctacaaccccgtagccaacgaaaaacaagagcgcgtccgcctcaatgcagaccgcctgaaccactggattgcacaaggcgcgcaagtcagcgactccgttgcaaaactgattaaagaacaaaaagccgtctaa

SEQ ID NO: 47 - DNA, 246 bp, Neisseria gonorrhoeae
ttagacggctttttgttctttaatcagttttgcaacggagtcgctgacttgcgcgccttgtgcaatccagtggttcaggcggtctgcattgaggcggacgcgctcttgtttttcgttggctacggggttgtagaagcctacgcgttcgatgaagcggccgtcgcggcggctgcgtgagtcagtaacgatgacgttgtagaaggggcggtgtttcgagccgccgcgtgccaaacggataactaccat

SEQ ID NO: 48 - RNA, 246 nt, Neisseria gonorrhoeae
augguaguuauccguuuggcacgcggcggcucgaaacaccgccccuucuacaacgucaucguuacugacucacgcagccgccgcgacggccgcuucaucgaacgcguaggcuucuacaaccccguagccaacgaaaaacaagagcgcguccgccucaaugcagaccgccugaaccacuggauugcacaaggcgcgcaagucagcgacuccguugcaaaacugauuaaagaacaaaaagccgucuaa

SEQ ID NO: 49 - DNA, 933 bp, Neisseria gonorrhoeae
atgaaaattgcaaacagcatcaccgaattgatcggcaacacgcctttggtcaaactgaaccgtttgaccaaaggtttgaaggcagaggttgccgtgaaactggaattttttaatccgggcagcagcgtcaaagaccgcattgccgaagcaatgatcgaggccgccgaaaaagcgggaaaaatcaacaaaaacaccgtcattgtcgaagcaaccagcggcaatacgggtatcggtttggcaatggtatgtgccgcacgcggctacaaactggcgattaccatgccggaaagcatgagcaaagagcgcaaaatgctgttgcgcacgtttggcgcggaactgattctaacccccgccgccgaaggtatggcgggcgcgattgccaaagcgcaatccttggtggacgctcatccagacacttattttatgccgcgccagttcgacaatgaggcaaatcccgaagtccaccgcaaaacaaccgccgaggaaatttggaacgataccgacggtaaagtcgatgtcttcgttgccggcgtcggcacgggcggtacgattaccggcgtgggcgaagtgttgaaaaaatacaaacccgaaattgaagtgtgtgccgtcgaagctggcgcttcccccgtattgagcggcggcgaaaaaggtccgcacccgattcaaggtatcggcgcaggttttattccgaccgttttgaataccaaaatctacgacagcattgccaaagtgccgaacgaagcggcttttgaaaccgcccgtgcaatggcggaaaaagaaggcattttggcgggcatttcttccggtgcggcggtttggagcgcgttgcagcttgccaaacagcctgaaaacgaaggcaagctgatagtcgtgctgctgccttcttatggcgaacgctatctttctacgccactttttgcagatttggcataa

SEQ ID NO: 50 - DNA, 933 bp, Neisseria gonorrhoeae
ttatgccaaatctgcaaaaagtggcgtagaaagatagcgttcgccataagaaggcagcagcacgactatcagcttgccttcgttttcaggctgtttggcaagctgcaacgcgctccaaaccgccgcaccggaagaaatgcccgccaaaatgccttctttttccgccattgcacgggcggtttcaaaagccgcttcgttcggcactttggcaatgctgtcgtagattttggtattcaaaacggtcggaataaaacctgcgccgataccttgaatcgggtgcggacctttttcgccgccgctcaatacgggggaagcgccagcttcgacggcacacacttcaatttcgggtttgtattttttcaacacttcgcccacgccggtaatcgtaccgcccgtgccgacgccggcaacgaagacatcgactttaccgtcggtatcgttccaaatttcctcggcggttgttttgcggtggacttcgggatttgcctcattgtcgaactggcgcggcataaaataagtgtctggatgagcgtccaccaaggattgcgctttggcaatcgcgcccgccataccttcggcggcgggggttagaatcagttccgcgccaaacgtgcgcaacagcattttgcgctctttgctcatgctttccggcatggtaatcgccagtttgtagccgcgtgcggcacataccattgccaaaccgatacccgtattgccgctggttgcttcgacaatgacggtgtttttgttgatttttcccgctttttcggcggcctcgatcattgcttcggcaatgcggtctttgacgctgctgcccggattaaaaaattccagtttcacggcaacctctgccttcaaacctttggtcaaacggttcagtttgaccaaaggcgtgttgccgatcaattcggtgatgctgtttgcaattttcat

SEQ ID NO: 51 - RNA, 933 nt, Neisseria gonorrhoeae
augaaaauugcaaacagcaucaccgaauugaucggcaacacgccuuuggucaaacugaaccguuugaccaaagguuugaaggcagagguugccgugaaacuggaauuuuuuaauccgggcagcagcgucaaagaccgcauugccgaagcaaugaucgaggccgccgaaaaagcgggaaaaaucaacaaaaacaccgucauugucgaagcaaccagcggcaauacggguaucgguuuggcaaugguaugugccgcacgcggcuacaaacuggcgauuaccaugccggaaagcaugagcaaagagcgcaaaaugcuguugcgcacguuuggcgcggaacugauucuaacccccgccgccgaagguauggcgggcgcgauugccaaagcgcaauccuugguggacgcucauccagacacuuauuuuaugccgcgccaguucgacaaugaggcaaaucccgaaguccaccgcaaaacaaccgccgaggaaauuuggaacgauaccgacgguaaagucgaugucuucguugccggcgucggcacgggcgguacgauuaccggcgugggcgaaguguugaaaaaauacaaacccgaaauugaagugugugccgucgaagcuggcgcuucccccguauugagcggcggcgaaaaagguccgcacccgauucaagguaucggcgcagguuuuauuccgaccguuuugaauaccaaaaucuacgacagcauugccaaagugccgaacgaagcggcuuuugaaaccgcccgugcaauggcggaaaaagaaggcauuuuggcgggcauuucuuccggugcggcgguuuggagcgcguugcagcuugccaaacagccugaaaacgaaggcaagcugauagucgugcugcugccuucuuauggcgaacgcuaucuuucuacgccacuuuuugcagauuuggcauaa

SEQ ID NO: 52 - DNA, 1314 bp, Neisseria gonorrhoeae
atgatgagcgtaactgttgaaattttagaaaatctggaacgcaaagtagtgttgtccctgccttggtccgaaatcaacgcagaaaccgataaaaaactgaaacaaacccaacgccgtgcaaaaatcgacggtttccgtccgggtaaagcacctttaaaaatgattgcccaaatgtacggtgcgagcgcgcaaaacgacgtgatcaacgagctggtgcaacgccgcttctacgatgttgccgttgcccaagagttgaaagtggcaggctatccacgttttgaaggcgttgaagaacaagacgataaagagtctttcaaagttgccgccatttttgaagtgttccccgaagtcgttatcggcgatttgtctgcacaagaggtcgaaaaagtaaccgcttccgtcggcgatgccgaagtcgaccaaactgtagaaatcctgcgtaaacaacgtacccgcttcaaccatgttgaccgcgaagcccgaaacggcgaccgcgtcatcatcgactttgaaggcaaaatcgacggcgaaccttttgccggcggcacatccaaaaactacgccttcgtattgggcgcaggtcaaatgctgcctgaatttgaagccggcgtagtcggcatgaaagcgggcgaaagtaaagacgttaccgtcaacttccctgaagaataccacggcaaagatgttgccggtaaaactgccgtgttcaccattacgctgaacaatgtttccgagcccactctgcctgaagtcgatgcagattttgcaaaagccttgggtattgcggatggcgatgttgccaaaatgcgtgaagaagtgaagaaaaacgtaagccgcgaagtggaacgccgcgtgaacgaacaaaccaaagaatccgtaatgaacgcgctgattaaagccgtagagttgaaagttccggttgctttggtcaatgaagaagccgcccgcctggcaaacgaaatgaaacaaaacttcgttaaccaaggtatgaccgatgccgcgaacttggatttgcctttggatatgttcaaagaacaagccgaacgtcgcgtatctttgggtctgattttggccaaactggttgacgaaaacaaactggaaccgactgaagggcaaatcaaagccgttgtcgccaacttcgcagaaagctacgaagatcctcaagaagtgattgactggtactacgcagatacttcccgcctgcaagccccgacttctttggcagtagaaagcaacgttgttgatttcgttttgggcaaagccaaagtaaacaaaaaagctttgtcttttgacgaagtgatgggcgcgcaagcctga

SEQ ID NO: 53 - DNA, 1314 bp, Neisseria gonorrhoeae
tcaggcttgcgcgcccatcacttcgtcaaaagacaaagcttttttgtttactttggctttgcccaaaacgaaatcaacaacgttgctttctactgccaaagaagtcggggcttgcaggcgggaagtatctgcgtagtaccagtcaatcacttcttgaggatcttcgtagctttctgcgaagttggcgacaacggctttgatttgcccttcagtcggttccagtttgttttcgtcaaccagtttggccaaaatcagacccaaagatacgcgacgttcggcttgttctttgaacatatccaaaggcaaatccaagttcgcggcatcggtcataccttggttaacgaagttttgtttcatttcgtttgccaggcgggcggcttcttcattgaccaaagcaaccggaactttcaactctacggctttaatcagcgcgttcattacggattctttggtttgttcgttcacgcggcgttccacttcgcggcttacgtttttcttcacttcttcacgcattttggcaacatcgccatccgcaatacccaaggcttttgcaaaatctgcatcgacttcaggcagagtgggctcggaaacattgttcagcgtaatggtgaacacggcagttttaccggcaacatctttgccgtggtattcttcagggaagttgacggtaacgtctttactttcgcccgctttcatgccgactacgccggcttcaaattcaggcagcatttgacctgcgcccaatacgaaggcgtagtttttggatgtgccgccggcaaaaggttcgccgtcgattttgccttcaaagtcgatgatgacgcggtcgccgtttcgggcttcgcggtcaacatggttgaagcgggtacgttgtttacgcaggatttctacagtttggtcgacttcggcatcgccgacggaagcggttactttttcgacctcttgtgcagacaaatcgccgataacgacttcggggaacacttcaaaaatggcggcaactttgaaagactctttatcgtcttgttcttcaacgccttcaaaacgtggatagcctgccactttcaactcttgggcaacggcaacatcgtagaagcggcgttgcaccagctcgttgatcacgtcgttttgcgcgctcgcaccgtacatttgggcaatcatttttaaaggtgctttacccggacggaaaccgtcgatttttgcacggcgttgggtttgtttcagttttttatcggtttctgcgttgatttcggaccaaggcagggacaacactactttgcgttccagattttctaaaatttcaacagttacgctcatcat

SEQ ID NO: 54 - RNA, 1314 nt, Neisseria gonorrhoeae
augaugagcguaacuguugaaauuuuagaaaaucuggaacgcaaaguaguguugucccugccuugguccgaaaucaacgcagaaaccgauaaaaaacugaaacaaacccaacgccgugcaaaaaucgacgguuuccguccggguaaagcaccuuuaaaaaugauugcccaaauguacggugcgagcgcgcaaaacgacgugaucaacgagcuggugcaacgccgcuucuacgauguugccguugcccaagaguugaaaguggcaggcuauccacguuuugaaggcguugaagaacaagacgauaaagagucuuucaaaguugccgccauuuuugaaguguuccccgaagucguuaucggcgauuugucugcacaagaggucgaaaaaguaaccgcuuccgucggcgaugccgaagucgaccaaacuguagaaauccugcguaaacaacguacccgcuucaaccauguugaccgcgaagcccgaaacggcgaccgcgucaucaucgacuuugaaggcaaaaucgacggcgaaccuuuugccggcggcacauccaaaaacuacgccuucguauugggcgcaggucaaaugcugccugaauuugaagccggcguagucggcaugaaagcgggcgaaaguaaagacguuaccgucaacuucccugaagaauaccacggcaaagauguugccgguaaaacugccguguucaccauuacgcugaacaauguuuccgagcccacucugccugaagucgaugcagauuuugcaaaagccuuggguauugcggauggcgauguugccaaaaugcgugaagaagugaagaaaaacguaagccgcgaaguggaacgccgcgugaacgaacaaaccaaagaauccguaaugaacgcgcugauuaaagccguagaguugaaaguuccgguugcuuuggucaaugaagaagccgcccgccuggcaaacgaaaugaaacaaaacuucguuaaccaagguaugaccgaugccgcgaacuuggauuugccuuuggauauguucaaagaacaagccgaacgucgcguaucuuugggucugauuuuggccaaacugguugacgaaaacaaacuggaaccgacugaagggcaaaucaaagccguugucgccaacuucgcagaaagcuacgaagauccucaagaagugauugacugguacuacgcagauacuucccgccugcaagccccgacuucuuuggcaguagaaagcaacguuguugauuucguuuugggcaaagccaaaguaaacaaaaaagcuuugucuuuugacgaagugaugggcgcgcaagccuga

SEQ ID NO: 55 - DNA, 615 bp, Neisseria gonorrhoeae
atgtcttttgataaccatcttgtccctaccgttatcgagcagagcggtcgcggtgagcgtgcattcgatatctattcccggcttttgaaagagcgcatcgtatttctggttggcccggtaaccgatgagtctgctaatctggtggtcgcccaactgttgtttttggaaagtgagaatccggataaggatattttcttctacatcaattcccccggcggctcggtaacggccggtatgtcgatttacgacacgatgaatttcatcaagcccgatgtatcgactttgtgcttggggcaggcggcaagtatgggcgcgttcttattgtcggcaggcgagaaaggcaaacgtttcgccctgcccaacagccggattatgattcaccagcctttaatcagcggcggcttgggcggtcaggcatccgacattgaaatccacgcacgcgagttgttgaaaatcaaagaaaaactcaaccgcctgatggcgaaacattgcggccgcgatttggcagatttggagcgcgacaccgaccgtgataatttcatgtctgccgaagaagcaaaagaatatggtttgatcgaccaagttttggaaaaccgcgcttctttgcggctttaa

SEQ ID NO: 56 - DNA, 615 bp, Neisseria gonorrhoeae
ttaaagccgcaaagaagcgcggttttccaaaacttggtcgatcaaaccatattcttttgcttcttcggcagacatgaaattatcacggtcggtgtcgcgctccaaatctgccaaatcgcggccgcaatgtttcgccatcaggcggttgagtttttctttgattttcaacaactcgcgtgcgtggatttcaatgtcggatgcctgaccgcccaagccgccgctgattaaaggctggtgaatcataatccggctgttgggcagggcgaaacgtttgcctttctcgcctgccgacaataagaacgcgcccatacttgccgcctgccccaagcacaaagtcgatacatcgggcttgatgaaattcatcgtgtcgtaaatcgacataccggccgttaccgagccgccgggggaattgatgtagaagaaaatatccttatccggattctcactttccaaaaacaacagttgggcgaccaccagattagcagactcatcggttaccgggccaaccagaaatacgatgcgctctttcaaaagccgggaatagatatcgaatgcacgctcaccgcgaccgctctgctcgataacggtagggacaagatggttatcaaaagacat

SEQ ID NO: 57 - RNA, 615 nt, Neisseria gonorrhoeae
augucuuuugauaaccaucuugucccuaccguuaucgagcagagcggucgcggugagcgugcauucgauaucuauucccggcuuuugaaagagcgcaucguauuucugguuggcccgguaaccgaugagucugcuaaucugguggucgcccaacuguuguuuuuggaaagugagaauccggauaaggauauuuucuucuacaucaauucccccggcggcucgguaacggccgguaugucgauuuacgacacgaugaauuucaucaagcccgauguaucgacuuugugcuuggggcaggcggcaaguaugggcgcguucuuauugucggcaggcgagaaaggcaaacguuucgcccugcccaacagccggauuaugauucaccagccuuuaaucagcggcggcuugggcggucaggcauccgacauugaaauccacgcacgcgaguuguugaaaaucaaagaaaaacucaaccgccugauggcgaaacauugcggccgcgauuuggcagauuuggagcgcgacaccgaccgugauaauuucaugucugccgaagaagcaaaagaauaugguuugaucgaccaaguuuuggaaaaccgcgcuucuuugcggcuuuaa

SEQ ID NO: 58 - DNA, 1686 bp, Neisseria gonorrhoeae
atgtctatggaaaattttgctcagctgttggaagaaagctttaccctgcaagaaatgaacccgggtgaggtgattaccgctgaagtagtggcaatcgaccaaaacttcgttaccgtaaacgcaggtctgaaatcagaatccctgatcgatgtagctgaattcaaaaacgctcaaggcgaaattgaagttaaagtcggcgacttcgttaccgttaccatcgaatccgtcgaaaacggcttcggcgaaaccaaactgtcccgcgaaaaagccaaacgcgcagccgattggatcgctttggaagaagccatggaaaacggcaacatcctgtccggcatcatcaacggtaaagtcaaaggcggcctgaccgttatgatcagcagcatccgcgcattcctgccgggttctttggtcgacgtacgtcccgttaaagacacttcccattttgaaggcaaagagatcgaattcaaagtgatcaaactggacaaaaaacgcaacaacgtcgttgtttcccgccgcgccgttttggaagccactttgggtgaagaacgcaaagccctgctggaaaacctgcaagaaggctccgtcatcaaaggcatcgtcaaaaatatcaccgactacggcgcattcgttgacctgggcggcatcgacggcctgctgcacatcaccgatttggcatggcgtcgcgtgaaacacccgagcgaagtcttggaagtcggtcaggaagttgaagccaaagtattgaaattcgaccaagaaaaacaacgtgtttccttgggtatgaaacaactgggcgaagatccttggagcggtctgacccgccgttatccgcaagccacccgcctgttcggcaaagtatccaacctgaccgactacggcgcattcgtcgaaatcgaacaaggcatcgaaggtttggtacacgtctccgaaatggactggaccaacaaaaacgtacacccgagcaaagtcgtacaactgggtgacgaagtcgaagtcatgattttggaaatcgacgaaggccgccgccgtatctctttgggtatgaaacaatgccaagccaatccttgggaagaatttgccgccaaccacaacaaaggagacaaaatctccggtgcggttaaatccattaccgatttcggcgtattcgtcggcctgcccggcggcatcgacggtctggttcacctgtccgacctgtcttggaccgaatccggcgaagaagccgtacgcaaatacaaaaaaggagaagaagtcgaagccgtcgtattggcaatcgatgtggaaaaagaacgcatctccttgggtatcaaacaactggaaggcgatcctttcggcaacttcatcagcgtgaacgacaaaggttctttggttaaaggttccgtgaaatctgttgacgccaaaggcgctgttatcgccctgtctgacgaagtagaaggctacctgcctgcttccgaatttgcagccgaccgcgttgaagacttgaccaccaaactgaaagaaggcgacgaagttgaagccgtcatcgttaccgttgaccgcaaaaaccgcagcatcaaactttccgttaaagccaaagatgccaaagaaagccgcgaagcactgaactccgtcaatgccgccgccaatgcgaatgccggtaccaccagcttgggcgacctgctgaaagccaaactctccggcgaacaagaataa

SEQ ID NO: 59 - DNA, 1686 bp, Neisseria gonorrhoeae
ttattcttgttcgccggagagtttggctttcagcaggtcgcccaagctggtggtaccggcattcgcattggcggcggcattgacggagttcagtgcttcgcggctttctttggcatctttggctttaacggaaagtttgatgctgcggtttttgcggtcaacggtaacgatgacggcttcaacttcgtcgccttctttcagtttggtggtcaagtcttcaacgcggtcggctgcaaattcggaagcaggcaggtagccttctacttcgtcagacagggcgataacagcgcctttggcgtcaacagatttcacggaacctttaaccaaagaacctttgtcgttcacgctgatgaagttgccgaaaggatcgccttccagttgtttgatacccaaggagatgcgttctttttccacatcgattgccaatacgacggcttcgacttcttctccttttttgtatttgcgtacggcttcttcgccggattcggtccaagacaggtcggacaggtgaaccagaccgtcgatgccgccgggcaggccgacgaatacgccgaaatcggtaatggatttaaccgcaccggagattttgtctcctttgttgtggttggcggcaaattcttcccaaggattggcttggcattgtttcatacccaaagagatacggcggcggccttcgtcgatttccaaaatcatgacttcgacttcgtcacccagttgtacgactttgctcgggtgtacgtttttgttggtccagtccatttcggagacgtgtaccaaaccttcgatgccttgttcgatttcgacgaatgcgccgtagtcggtcaggttggatactttgccgaacaggcgggtggcttgcggataacggcgggtcagaccgctccaaggatcttcgcccagttgtttcatacccaaggaaacacgttgtttttcttggtcgaatttcaatactttggcttcaacttcctgaccgacttccaagacttcgctcgggtgtttcacgcgacgccatgccaaatcggtgatgtgcagcaggccgtcgatgccgcccaggtcaacgaatgcgccgtagtcggtgatatttttgacgatgcctttgatgacggagccttcttgcaggttttccagcagggctttgcgttcttcacccaaagtggcttccaaaacggcgcggcgggaaacaacgacgttgttgcgttttttgtccagtttgatcactttgaattcgatctctttgccttcaaaatgggaagtgtctttaacgggacgtacgtcgaccaaagaacccggcaggaatgcgcggatgctgctgatcataacggtcaggccgcctttgactttaccgttgatgatgccggacaggatgttgccgttttccatggcttcttccaaagcgatccaatcggctgcgcgtttggctttttcgcgggacagtttggtttcgccgaagccgttttcgacggattcgatggtaacggtaacgaagtcgccgactttaacttcaatttcgccttgagcgtttttgaattcagctacatcgatcagggattctgatttcagacctgcgtttacggtaacgaagttttggtcgattgccactacttcagcggtaatcacctcacccgggttcatttcttgcagggtaaagctttcttccaacagctgagcaaaattttccatagacat

SEQ ID NO: 60 - RNA, 1686 nt, Neisseria gonorrhoeae
augucuauggaaaauuuugcucagcuguuggaagaaagcuuuacccugcaagaaaugaacccgggugaggugauuaccgcugaaguaguggcaaucgaccaaaacuucguuaccguaaacgcaggucugaaaucagaaucccugaucgauguagcugaauucaaaaacgcucaaggcgaaauugaaguuaaagucggcgacuucguuaccguuaccaucgaauccgucgaaaacggcuucggcgaaaccaaacugucccgcgaaaaagccaaacgcgcagccgauuggaucgcuuuggaagaagccauggaaaacggcaacauccuguccggcaucaucaacgguaaagucaaaggcggccugaccguuaugaucagcagcauccgcgcauuccugccggguucuuuggucgacguacgucccguuaaagacacuucccauuuugaaggcaaagagaucgaauucaaagugaucaaacuggacaaaaaacgcaacaacgucguuguuucccgccgcgccguuuuggaagccacuuugggugaagaacgcaaagcccugcuggaaaaccugcaagaaggcuccgucaucaaaggcaucgucaaaaauaucaccgacuacggcgcauucguugaccugggcggcaucgacggccugcugcacaucaccgauuuggcauggcgucgcgugaaacacccgagcgaagucuuggaagucggucaggaaguugaagccaaaguauugaaauucgaccaagaaaaacaacguguuuccuuggguaugaaacaacugggcgaagauccuuggagcggucugacccgccguuauccgcaagccacccgccuguucggcaaaguauccaaccugaccgacuacggcgcauucgucgaaaucgaacaaggcaucgaagguuugguacacgucuccgaaauggacuggaccaacaaaaacguacacccgagcaaagucguacaacugggugacgaagucgaagucaugauuuuggaaaucgacgaaggccgccgccguaucucuuuggguaugaaacaaugccaagccaauccuugggaagaauuugccgccaaccacaacaaaggagacaaaaucuccggugcgguuaaauccauuaccgauuucggcguauucgucggccugcccggcggcaucgacggucugguucaccuguccgaccugucuuggaccgaauccggcgaagaagccguacgcaaauacaaaaaaggagaagaagucgaagccgucguauuggcaaucgauguggaaaaagaacgcaucuccuuggguaucaaacaacuggaaggcgauccuuucggcaacuucaucagcgugaacgacaaagguucuuugguuaaagguuccgugaaaucuguugacgccaaaggcgcuguuaucgcccugucugacgaaguagaaggcuaccugccugcuuccgaauuugcagccgaccgcguugaagacuugaccaccaaacugaaagaaggcgacgaaguugaagccgucaucguuaccguugaccgcaaaaaccgcagcaucaaacuuuccguuaaagccaaagaugccaaagaaagccgcgaagcacugaacuccgucaaugccgccgccaaugcgaaugccgguaccaccagcuugggcgaccugcugaaagccaaacucuccggcgaacaagaauaa

SEQ ID NO: 61 - DNA, 441 bp, Neisseria gonorrhoeae
atgatgcagactttccgaaaaatcagcctgtatgccgcaaccttgtggctcggtatgcagattatggcaggttatatcgccgcaccggtgctgttcaaaatgctgcccaaaatgcaggcgggcgaaattgccggcgtattgttcgacatcctctcttggagcgggcttgccgtttggggcacggtactggctgccgcctttgccgccctaacccggcggcaaaccgccctgctgctttttttattgtccgcccttgccgccaaccaatttttggttacacccgttatcgaggcactgaaatacgggcatgaaaattggctgttgtcggttgcaggcggatccttcggaatgtggcacggtatttccagcatgactttcatggcaaccgccctactttcagcagttttaagttggcggctttccggcaaagaggccgtctga

SEQ ID NO: 62 - DNA, 441 bp, Neisseria gonorrhoeae
tcagacggcctctttgccggaaagccgccaacttaaaactgctgaaagtagggcggttgccatgaaagtcatgctggaaataccgtgccacattccgaaggatccgcctgcaaccgacaacagccaattttcatgcccgtatttcagtgcctcgataacgggtgtaaccaaaaattggttggcggcaagggcggacaataaaaaaagcagcagggcggtttgccgccgggttagggcggcaaaggcggcagccagtaccgtgccccaaacggcaagcccgctccaagagaggatgtcgaacaatacgccggcaatttcgcccgcctgcattttgggcagcattttgaacagcaccggtgcggcgatataacctgccataatctgcataccgagccacaaggttgcggcatacaggctgatttttcggaaagtctgcatcat

SEQ ID NO: 63 - RNA, 441 nt, Neisseria gonorrhoeae
augaugcagacuuuccgaaaaaucagccuguaugccgcaaccuuguggcucgguaugcagauuauggcagguuauaucgccgcaccggugcuguucaaaaugcugcccaaaaugcaggcgggcgaaauugccggcguauuguucgacauccucucuuggagcgggcuugccguuuggggcacgguacuggcugccgccuuugccgcccuaacccggcggcaaaccgcccugcugcuuuuuuuauuguccgcccuugccgccaaccaauuuuugguuacacccguuaucgaggcacugaaauacgggcaugaaaauuggcuguugucgguugcaggcggauccuucggaauguggcacgguauuuccagcaugacuuucauggcaaccgcccuacuuucagcaguuuuaaguuggcggcuuuccggcaaagaggccgucuga

SEQ ID NO: 64 - DNA, 843 bp, Neisseria gonorrhoeae
atggatattaaaatcaacgacatcaccctcggcaacaattcgcctttcgtcctattcggcggcatcaacgttttagaagatttggattccaccctccaaacctgtgcgcattacgtcgaagttacccgcaaactgggcatcccctatatctttaaagcctctttcgacaaggcaaaccgctcgtctatccattcctatcgcggcgtaggcttggaagaaggcttaaagatttttgaaaaagtcaaagcagagttcggcatccccgtcattaccgacgtacacgaaccccatcaatgccaacccgtcgccgaagtgtgcgatgtcatccagcttcccgcctttcttgcgcggcagaccgatttggtggccgcaatggcggaaacgggcaatgttatcaacatcaaaaaaccccagttcctcagcccttcgcaaatgaaaaacatcgtggaaaaattccgcgaagccggcaacgggaagctgattttatgcgaacgcggcagcagcttcggctacgacaacctcgttgtcgatatgctcggtttcggcgtgatgaaacaaacctgcggcaacctgccggttattttcgacgttacccattccctgcaaacccgcgatgccggttctgccgcatccggcggtcgtcgcgcacaggctttggatttggcacttgcaggcatggcaacccgccttgccggcctgttcctcgaatcgcaccccgatccgaaactggcaaaatgcgacggccccagcgcgctgccgctacaccttttagaaaattttttaatccgcatcaaagcattggacgatttaatcaaatcacaaccgattttaacaatcgagtaa

SEQ ID NO: 65 - DNA, 843 bp, Neisseria gonorrhoeae
ttactcgattgttaaaatcggttgtgatttgattaaatcgtccaatgctttgatgcggattaaaaaattttctaaaaggtgtagcggcagcgcgctggggccgtcgcattttgccagtttcggatcggggtgcgattcgaggaacaggccggcaaggcgggttgccatgcctgcaagtgccaaatccaaagcctgtgcgcgacgaccgccggatgcggcagaaccggcatcgcgggtttgcagggaatgggtaacgtcgaaaataaccggcaggttgccgcaggtttgtttcatcacgccgaaaccgagcatatcgacaacgaggttgtcgtagccgaagctgctgccgcgttcgcataaaatcagcttcccgttgccggcttcgcggaatttttccacgatgtttttcatttgcgaagggctgaggaactggggttttttgatgttgataacattgcccgtttccgccattgcggccaccaaatcggtctgccgcgcaagaaaggcgggaagctggatgacatcgcacacttcggcgacgggttggcattgatggggttcgtgtacgtcggtaatgacggggatgccgaactctgctttgactttttcaaaaatctttaagccttcttccaagcctacgccgcgataggaatggatagacgagcggtttgccttgtcgaaagaggctttaaagatataggggatgcccagtttgcgggtaacttcgacgtaatgcgcacaggtttggagggtggaatccaaatcttctaaaacgttgatgccgccgaataggacgaaaggcgaattgttgccgagggtgatgtcgttgattttaatatccat

SEQ ID NO: 66 - RNA, 843 nt, Neisseria gonorrhoeae
auggauauuaaaaucaacgacaucacccucggcaacaauucgccuuucguccuauucggcggcaucaacguuuuagaagauuuggauuccacccuccaaaccugugcgcauuacgucgaaguuacccgcaaacugggcauccccuauaucuuuaaagccucuuucgacaaggcaaaccgcucgucuauccauuccuaucgcggcguaggcuuggaagaaggcuuaaagauuuuugaaaaagucaaagcagaguucggcauccccgucauuaccgacguacacgaaccccaucaaugccaacccgucgccgaagugugcgaugucauccagcuucccgccuuucuugcgcggcagaccgauuugguggccgcaauggcggaaacgggcaauguuaucaacaucaaaaaaccccaguuccucagcccuucgcaaaugaaaaacaucguggaaaaauuccgcgaagccggcaacgggaagcugauuuuaugcgaacgcggcagcagcuucggcuacgacaaccucguugucgauaugcucgguuucggcgugaugaaacaaaccugcggcaaccugccgguuauuuucgacguuacccauucccugcaaacccgcgaugccgguucugccgcauccggcggucgucgcgcacaggcuuuggauuuggcacuugcaggcauggcaacccgccuugccggccuguuccucgaaucgcaccccgauccgaaacuggcaaaaugcgacggccccagcgcgcugccgcuacaccuuuuagaaaauuuuuuaauccgcaucaaagcauuggacgauuuaaucaaaucacaaccgauuuuaacaaucgaguaa

SEQ ID NO: 67 - DNA, 384 bp, Neisseria gonorrhoeae
atgttccgtactatacttggcggaaaaatccaccgcgccaccgtaaccgaagccgatttaaactacgtcggcagcattaccgtcgatcaagacctgttagacgcggcaggcatctgccccaacgaaaaagtcgccatcgtcaacaacaacaacggcgaacgttttgaaacctataccattgcagggaaacgcggcagcggcgtgatttgcctgaacggtgctgcagccaggctggtacagaaaggcgacatcgtcatcattatgtcttatatccaactttccgaaccggaaatcgccgcacacgaacccaaagtcgtcttagtggacggaaacaataaaatccgcgacatcatctcctacgagccgccgcacaccgtactgtaa

SEQ ID NO: 68 - DNA, 384 bp, Neisseria gonorrhoeae
ttacagtacggtgtgcggcggctcgtaggagatgatgtcgcggattttattgtttccgtccactaagacgactttgggttcgtgtgcggcgatttccggttcggaaagttggatataagacataatgatgacgatgtcgcctttctgtaccagcctggctgcagcaccgttcaggcaaatcacgccgctgccgcgtttccctgcaatggtataggtttcaaaacgttcgccgttgttgttgttgacgatggcgactttttcgttggggcagatgcctgccgcgtctaacaggtcttgatcgacggtaatgctgccgacgtagtttaaatcggcttcggttacggtggcgcggtggatttttccgccaagtatagtacggaacat

SEQ ID NO: 69 - RNA, 384 nt, Neisseria gonorrhoeae
auguuccguacuauacuuggcggaaaaauccaccgcgccaccguaaccgaagccgauuuaaacuacgucggcagcauuaccgucgaucaagaccuguuagacgcggcaggcaucugccccaacgaaaaagucgccaucgucaacaacaacaacggcgaacguuuugaaaccuauaccauugcagggaaacgcggcagcggcgugauuugccugaacggugcugcagccaggcugguacagaaaggcgacaucgucaucauuaugucuuauauccaacuuuccgaaccggaaaucgccgcacacgaacccaaagucgucuuaguggacggaaacaauaaaauccgcgacaucaucuccuacgagccgccgcacaccguacuguaa

SEQ ID NO: 70 - DNA, 555 bp, Neisseria gonorrhoeae
atgaaaaaaatcatcgcctccgcgcttatcgcaacattcgcactcaccgcctgccaagacgacacgcaggcgcggctcgaacggcagcagaaacagattgaagccctgcaacagcagctcgcacagcaggcagacgatacggtttaccaactgactcccgaagcagtcaaagacaccattcctgcccaggcgcaggcaaacggcaacaacggtcagcccgttaccggcaaagacgggcagcagtatatttacgaccaatcgacaggaagctggctgctgcaaagcctgattggcgcggcggcaggcgcgtttatcggcaacgcgctggcaaacaaattcacacgggcgggcaaccaagacagccccgtcgcccgtcgcgcgcgtgctgcctaccatcagtccgcacgccccaatgcgcgcaccagcagggatttgaacacgcgcagcctccgtgcaaaacaacaggcggcgcaggcgcagcgttaccgcccgacaacgcgcccgcccgtcaattaccgccgtcccgctatgcgcggtttcggcagaaggcggtaa

SEQ ID NO: 71 - DNA, 555 bp, Neisseria gonorrhoeae
ttaccgccttctgccgaaaccgcgcatagcgggacggcggtaattgacgggcgggcgcgttgtcgggcggtaacgctgcgcctgcgccgcctgttgttttgcacggaggctgcgcgtgttcaaatccctgctggtgcgcgcattggggcgtgcggactgatggtaggcagcacgcgcgcgacgggcgacggggctgtcttggttgcccgcccgtgtgaatttgtttgccagcgcgttgccgataaacgcgcctgccgccgcgccaatcaggctttgcagcagccagcttcctgtcgattggtcgtaaatatactgctgcccgtctttgccggtaacgggctgaccgttgttgccgtttgcctgcgcctgggcaggaatggtgtctttgactgcttcgggagtcagttggtaaaccgtatcgtctgcctgctgtgcgagctgctgttgcagggcttcaatctgtttctgctgccgttcgagccgcgcctgcgtgtcgtcttggcaggcggtgagtgcgaatgttgcgataagcgcggaggcgatgatttttttcat

SEQ ID NO: 72 - RNA, 555 nt, Neisseria gonorrhoeae
augaaaaaaaucaucgccuccgcgcuuaucgcaacauucgcacucaccgccugccaagacgacacgcaggcgcggcucgaacggcagcagaaacagauugaagcccugcaacagcagcucgcacagcaggcagacgauacgguuuaccaacugacucccgaagcagucaaagacaccauuccugcccaggcgcaggcaaacggcaacaacggucagcccguuaccggcaaagacgggcagcaguauauuuacgaccaaucgacaggaagcuggcugcugcaaagccugauuggcgcggcggcaggcgcguuuaucggcaacgcgcuggcaaacaaauucacacgggcgggcaaccaagacagccccgucgcccgucgcgcgcgugcugccuaccaucaguccgcacgccccaaugcgcgcaccagcagggauuugaacacgcgcagccuccgugcaaaacaacaggcggcgcaggcgcagcguuaccgcccgacaacgcgcccgcccgucaauuaccgccgucccgcuaugcgcgguuucggcagaaggcgguaa

SEQ ID NO: 73 - DNA, 729 bp, Neisseria gonorrhoeae
atggcaggccatagcaagtgggcgaatatccagcataaaaaagcccgtcaggatgccaaacgcggcaaaatcttcacccgtttaatcaaagaaatcaccgttgcggcgcgtatgggcggcggcgatcccggcgcaaatccgcgcctgcgtctggctttggaaaaagcagccgaaaacaatatgcccaaagacaatgtgcaacgcgccatcgacaaaggtacgggtaacttggaaggcgtggaatacatcgagttgcgctacgaaggctacggcatcggcggcgcagctttgatggtggactgcctgaccgacaacaaaacccgcaccgttgcggacgtacgccacgcatttaccaaaaacggcggcaacttgggtaccgacggctgcgtggcgttcaacttcgtgcatcagggctatttggtattcgaacccggcgttgacgaagacgagctgatggaagcggctttggaagccggtgcggaagacgtggttaccaacgacgacggttccatcgaagtcattaccgcgccaaatgattgggcgggcgtaaaatccgctttggaggcggcaggttacaaatccgttgacggcgacgttacgatgcgcgcccaaaacgaaaccgaactctccggcgacgatgccgtcaaaatgcaaaaactgattgacgcgctggaagacttggacgacgtgcaagacgtttacacttccgccgtattgaatctggactga

SEQ ID NO: 74 - DNA, 729 bp, Neisseria gonorrhoeae
tcagtccagattcaatacggcggaagtgtaaacgtcttgcacgtcgtccaagtcttccagcgcgtcaatcagtttttgcattttgacggcatcgtcgccggagagttcggtttcgttttgggcgcgcatcgtaacgtcgccgtcaacggatttgtaacctgccgcctccaaagcggattttacgcccgcccaatcatttggcgcggtaatgacttcgatggaaccgtcgtcgttggtaaccacgtcttccgcaccggcttccaaagccgcttccatcagctcgtcttcgtcaacgccgggttcgaataccaaatagccctgatgcacgaagttgaacgccacgcagccgtcggtacccaagttgccgccgtttttggtaaatgcgtggcgtacgtccgcaacggtgcgggttttgttgtcggtcaggcagtccaccatcaaagctgcgccgccgatgccgtagccttcgtagcgcaactcgatgtattccacgccttccaagttacccgtacctttgtcgatggcgcgttgcacattgtctttgggcatattgttttcggctgctttttccaaagccagacgcaggcgcggatttgcgccgggatcgccgccgcccatacgcgccgcaacggtgatttctttgattaaacgggtgaagattttgccgcgtttggcatcctgacgggcttttttatgctggatattcgcccacttgctatggcctgccat

SEQ ID NO: 75 - RNA, 729 nt, Neisseria gonorrhoeae
auggcaggccauagcaagugggcgaauauccagcauaaaaaagcccgucaggaugccaaacgcggcaaaaucuucacccguuuaaucaaagaaaucaccguugcggcgcguaugggcggcggcgaucccggcgcaaauccgcgccugcgucuggcuuuggaaaaagcagccgaaaacaauaugcccaaagacaaugugcaacgcgccaucgacaaagguacggguaacuuggaaggcguggaauacaucgaguugcgcuacgaaggcuacggcaucggcggcgcagcuuugaugguggacugccugaccgacaacaaaacccgcaccguugcggacguacgccacgcauuuaccaaaaacggcggcaacuuggguaccgacggcugcguggcguucaacuucgugcaucagggcuauuugguauucgaacccggcguugacgaagacgagcugauggaagcggcuuuggaagccggugcggaagacgugguuaccaacgacgacgguuccaucgaagucauuaccgcgccaaaugauugggcgggcguaaaauccgcuuuggaggcggcagguuacaaauccguugacggcgacguuacgaugcgcgcccaaaacgaaaccgaacucuccggcgacgaugccgucaaaaugcaaaaacugauugacgcgcuggaagacuuggacgacgugcaagacguuuacacuuccgccguauugaaucuggacuga

SEQ ID NO: 76 - DNA, 1179 bp, Neisseria gonorrhoeae
atggcaaaaatgatgaaatgggcggctgttgcggcggtcgcggcggcagcggtttggggcggatggtcttatctgaagcccgaaccgcaggctgcttatattacggaaacggtcaggcgcggcgatatcagccggacggtttccgcgacgggcgagatttcgccgtccaacctggtatcggtcggcgcgcaggcttcggggcagattaaaaagctttatgtcaaactcgggcaacaggtcaaaaagggcgatttgattgcggaaatcaattcgaccacgcagaccaacacgatcgatatggaaaaatccaaattggaaacgtatcaggcgaagctggtgtcggcacagattgcattgggcagcgcggagaagaaatataagcgtcaggcggcgttgtggaaggatgatgcgacctctaaagaagatttggaaagcgcgcaggatgcgcttgccgccgccaaagccaatgttgccgagttgaaggctttaatcagacagagcaaaatttccatcaataccgccgagtcggatttgggctacacgcgcattaccgcgacgatggacggcacggtggtggcgattcccgtggaagaggggcagactgtgaacgcggcgcagtctacgccgacgattgtccaattggcgaatctggatatgatgttgaacaaaatgcagattgccgagggcgatattaccaaggtgaaggcggggcaggatatttcgtttacgattttgtccgaaccggatacgccgattaaggcgaagctcgacagcgtcgaccccgggctgaccacgatgtcgtcgggcggctacaacagcagtacggatacggcttccaatgcggtctattattatgcccgttcgtttgtgccgaatccggacggcaaactcgccacggggatgacgacgcagaatacggttgaaatcgacggtgtgaaaaatgtgttgcttattccgtcgctgaccgtgaaaaatcgcggcggcaaggcgttcgtacgcgtgttgggtgcggacggcaaggcagtggaacgcgaaatccggaccggtatgaaagacagtatgaataccgaagtgaaaagcgggttgaaagagggggacaaagtggtcatctccgaaataaccgccgccgagcagcaggaaagcggcgaacgcgccctaggcggcccgccgcgccgataa

SEQ ID NO: 77 - DNA, 1179 bp, Neisseria gonorrhoeae
ttatcggcgcggcgggccgcctagggcgcgttcgccgctttcctgctgctcggcggcggttatttcggagatgaccactttgtccccctctttcaacccgcttttcacttcggtattcatactgtctttcataccggtccggatttcgcgttccactgccttgccgtccgcacccaacacgcgtacgaacgccttgccgccgcgatttttcacggtcagcgacggaataagcaacacatttttcacaccgtcgatttcaaccgtattctgcgtcgtcatccccgtggcgagtttgccgtccggattcggcacaaacgaacgggcataataatagaccgcattggaagccgtatccgtactgctgttgtagccgcccgacgacatcgtggtcagcccggggtcgacgctgtcgagcttcgccttaatcggcgtatccggttcggacaaaatcgtaaacgaaatatcctgccccgccttcaccttggtaatatcgccctcggcaatctgcattttgttcaacatcatatccagattcgccaattggacaatcgtcggcgtagactgcgccgcgttcacagtctgcccctcttccacgggaatcgccaccaccgtgccgtccatcgtcgcggtaatgcgcgtgtagcccaaatccgactcggcggtattgatggaaattttgctctgtctgattaaagccttcaactcggcaacattggctttggcggcggcaagcgcatcctgcgcgctttccaaatcttctttagaggtcgcatcatccttccacaacgccgcctgacgcttatatttcttctccgcgctgcccaatgcaatctgtgccgacaccagcttcgcctgatacgtttccaatttggatttttccatatcgatcgtgttggtctgcgtggtcgaattgatttccgcaatcaaatcgccctttttgacctgttgcccgagtttgacataaagctttttaatctgccccgaagcctgcgcgccgaccgataccaggttggacggcgaaatctcgcccgtcgcggaaaccgtccggctgatatcgccgcgcctgaccgtttccgtaatataagcagcctgcggttcgggcttcagataagaccatccgccccaaaccgctgccgccgcgaccgccgcaacagccgcccatttcatcatttttgccat

SEQ ID NO: 78 - RNA, 1179 nt, Neisseria gonorrhoeae
auggcaaaaaugaugaaaugggcggcuguugcggcggucgcggcggcagcgguuuggggcggauggucuuaucugaagcccgaaccgcaggcugcuuauauuacggaaacggucaggcgcggcgauaucagccggacgguuuccgcgacgggcgagauuucgccguccaaccugguaucggucggcgcgcaggcuucggggcagauuaaaaagcuuuaugucaaacucgggcaacaggucaaaaagggcgauuugauugcggaaaucaauucgaccacgcagaccaacacgaucgauauggaaaaauccaaauuggaaacguaucaggcgaagcuggugucggcacagauugcauugggcagcgcggagaagaaauauaagcgucaggcggcguuguggaaggaugaugcgaccucuaaagaagauuuggaaagcgcgcaggaugcgcuugccgccgccaaagccaauguugccgaguugaaggcuuuaaucagacagagcaaaauuuccaucaauaccgccgagucggauuugggcuacacgcgcauuaccgcgacgauggacggcacggugguggcgauucccguggaagaggggcagacugugaacgcggcgcagucuacgccgacgauuguccaauuggcgaaucuggauaugauguugaacaaaaugcagauugccgagggcgauauuaccaaggugaaggcggggcaggauauuucguuuacgauuuuguccgaaccggauacgccgauuaaggcgaagcucgacagcgucgaccccgggcugaccacgaugucgucgggcggcuacaacagcaguacggauacggcuuccaaugcggucuauuauuaugcccguucguuugugccgaauccggacggcaaacucgccacggggaugacgacgcagaauacgguugaaaucgacggugugaaaaauguguugcuuauuccgucgcugaccgugaaaaaucgcggcggcaaggcguucguacgcguguugggugcggacggcaaggcaguggaacgcgaaauccggaccgguaugaaagacaguaugaauaccgaagugaaaagcggguugaaagagggggacaaaguggucaucuccgaaauaaccgccgccgagcagcaggaaagcggcgaacgcgcccuaggcggcccgccgcgccgauaa

SEQ ID NO: 79 - DNA, 291 bp, Neisseria gonorrhoeae
gtggaatattttatgttgctggcaacagacggggaggatgtgcatgaagcgcgtatggcggcacgtcccgaacactttaaacggctggaaacgctgaaatcggaaggccgtctgctgacggcaggcccaaacctgctgccggacaatcccgaacgtgtttcgggcagcttgattgtggcacagttcgagtctttggatgcggcgcaggcttgggctgaagacgatccctatgttcatgccggcgtgtacagcgaagtgctgatcaagccgtttaaagcggtgttcaaataa

SEQ ID NO: 80 - DNA, 291 bp, Neisseria gonorrhoeae
ttatttgaacaccgctttaaacggcttgatcagcacttcgctgtacacgccggcatgaacatagggatcgtcttcagcccaagcctgcgccgcatccaaagactcgaactgtgccacaatcaagctgcccgaaacacgttcgggattgtccggcagcaggtttgggcctgccgtcagcagacggccttccgatttcagcgtttccagccgtttaaagtgttcgggacgtgccgccatacgcgcttcatgcacatcctccccgtctgttgccagcaacataaaatattccac

SEQ ID NO: 81 - RNA, 291 nt, Neisseria gonorrhoeae
guggaauauuuuauguugcuggcaacagacggggaggaugugcaugaagcgcguauggcggcacgucccgaacacuuuaaacggcuggaaacgcugaaaucggaaggccgucugcugacggcaggcccaaaccugcugccggacaaucccgaacguguuucgggcagcuugauuguggcacaguucgagucuuuggaugcggcgcaggcuugggcugaagacgaucccuauguucaugccggcguguacagcgaagugcugaucaagccguuuaaagcgguguucaaauaa

SEQ ID NO: 82 - DNA, 531 bp, Neisseria gonorrhoeae
atgaaatttgtcagcgaccttttgtccgtcatcttgttttttgctacttataccgttaccaaaaatatgattgccgctgcggcggttgccttggttgcaggcgtggttcaggcggctttcctgtattggaagcataaaaggctggatacgatgcagtgggtcggactggtgctgattgtcgtattcggcggcgcaaccattgttttgggcgacagccgcttcattatgtggaagccgacagtattgttctggtgcggggcgttattcctgctgggcagccaccttgcgggtaaaaacggcttgaaagcgagtatcggcagggagattcagcttccggatgccgtatggggaaaattgacatatatgtgggtcggttttctgatttttatgggtattgccaactggtttgtgtttactaggtttgaagcgcaatgggttaactataagatgttcggttcgactgcgctgatgctttttttctttattattcagggtatttatctgagtacctatctgaaaaaggaggattga

SEQ ID NO: 83 - DNA, 531 bp, Neisseria gonorrhoeae
tcaatcctcctttttcagataggtactcagataaataccctgaataataaagaaaaaaagcatcagcgcagtcgaaccgaacatcttatagttaacccattgcgcttcaaacctagtaaacacaaaccagttggcaatacccataaaaatcagaaaaccgacccacatatatgtcaattttccccatacggcatccggaagctgaatctccctgccgatactcgctttcaagccgtttttacccgcaaggtggctgcccagcaggaataacgccccgcaccagaacaatactgtcggcttccacataatgaagcggctgtcgcccaaaacaatggttgcgccgccgaatacgacaatcagcaccagtccgacccactgcatcgtatccagccttttatgcttccaatacaggaaagccgcctgaaccacgcctgcaaccaaggcaaccgccgcagcggcaatcatatttttggtaacggtataagtagcaaaaaacaagatgacggacaaaaggtcgctgacaaatttcat

SEQ ID NO: 84 - RNA, 531 nt, Neisseria gonorrhoeae
augaaauuugucagcgaccuuuuguccgucaucuuguuuuuugcuacuuauaccguuaccaaaaauaugauugccgcugcggcgguugccuugguugcaggcgugguucaggcggcuuuccuguauuggaagcauaaaaggcuggauacgaugcagugggucggacuggugcugauugucguauucggcggcgcaaccauuguuuugggcgacagccgcuucauuauguggaagccgacaguauuguucuggugcggggcguuauuccugcugggcagccaccuugcggguaaaaacggcuugaaagcgaguaucggcagggagauucagcuuccggaugccguauggggaaaauugacauauaugugggucgguuuucugauuuuuauggguauugccaacugguuuguguuuacuagguuugaagcgcaauggguuaacuauaagauguucgguucgacugcgcugaugcuuuuuuucuuuauuauucaggguauuuaucugaguaccuaucugaaaaaggaggauuga

SEQ ID NO: 85 - DNA, 1677 bp, Neisseria gonorrhoeae
atgagcgtaggtttgctgaggattctggttcaaaaccaggtggttactgttgagcgggccgagcattactacaatgagtcgcaggcgggtaaggaagtgttgccgatgctgttttcagacggtgtcatttcgcccaagtcgcttgcggcattgattgcgagggtgttcagttattcgattcttgatttgcgtcattatccgcgccacagggtgctgatgggggtgttgacggaggagcagatggtggagttccactgtgtgccggttttccgtcggggcgacaaagtattttttgcggtttccgatccgacccagatgccgcaaattcagaaaaccgtttctgccgcagggattgcggttgagttggtcattgtcgaggatgaccagttggcgggtttgctcgattgggtgggttcgcgttcgacatcgctgcttcaggagcttggggaggggcaagaggaagaggaaagccacaccctgtatatcgacaacgaggaggcagaagacggccctgttccgaggtttatccataaaactttgtcggatgccttgcgtagcggggcatccgacatccatttcgagttttacgaacacaatgcgcgtatccgtttccgtgtggacgggcagctccgcgaggtggttcagccgcccattgcggtaagggggcagcttgcttcccggattaaggtaatgtcgcgtttggacatttccgaaaaacggataccgcaggacggtaggatgcagctgacctttcaaaagggcggcaagcctgtcgatttccgtgtcagcacattgccgacgctgtttggcgaaaaggtcgtgatgcggattttgaattccgatgccgcgtctttgaacatcgaccagctcggttttgagccgttccagaaaaaattgttgttggaagcgattcaccgtccttacgggatggtgctggtaaccggtccgacgggttcgggtaagacggtgtcgctctatacctgtttgaatattttgaatacggagtcggtaaatattgcaacggcggaagaccctgccgagattaacctgccgggcatcaatcaggttaacgtcaatgataagcagggtctgacttttgccgctgctttgaagtctttcctgcgtcaggacccggacatcattatggtcggtgagattcgtgatttggaaactgccgatattgcgattaaggcggcacaaacagggcatatggtgttttccacactgcacacgaataatgcgccggcgacgttgtcgcgtatgctgaatatgggtgtcgcgccgtttaatattgccagttcggtcagcctgattatggcgcagcgtcttttacgcaggctgtgttcgagctgcaaacaggaagtggaacgcccgtctgcctctgctttgaaggaagtcggtttcaccgatgaggatcttgcaaaagattggaaactttaccgcgccgtcggttgcgaccgttgccgggggcagggttataaggggcgtgcgggcgtgtatgaggttatgcccatcagcgaagaaatgcagcgtgtgattatgaacaacggtacggaagtgggtattttggacgttgcctataaggagggtatggtggatttgcgccgggccggtattttgaaaattatgcagggcattacttcattggaagaggtaacggcaaataccaacgattag

SEQ ID NO: 86 - DNA, 1677 bp, Neisseria gonorrhoeae
ctaatcgttggtatttgccgttacctcttccaatgaagtaatgccctgcataattttcaaaataccggcccggcgcaaatccaccataccctccttataggcaacgtccaaaatacccacttccgtaccgttgttcataatcacacgctgcatttcttcgctgatgggcataacctcatacacgcccgcacgccccttataaccctgcccccggcaacggtcgcaaccgacggcgcggtaaagtttccaatcttttgcaagatcctcatcggtgaaaccgacttccttcaaagcagaggcagacgggcgttccacttcctgtttgcagctcgaacacagcctgcgtaaaagacgctgcgccataatcaggctgaccgaactggcaatattaaacggcgcgacacccatattcagcatacgcgacaacgtcgccggcgcattattcgtgtgcagtgtggaaaacaccatatgccctgtttgtgccgccttaatcgcaatatcggcagtttccaaatcacgaatctcaccgaccataatgatgtccgggtcctgacgcaggaaagacttcaaagcagcggcaaaagtcagaccctgcttatcattgacgttaacctgattgatgcccggcaggttaatctcggcagggtcttccgccgttgcaatatttaccgactccgtattcaaaatattcaaacaggtatagagcgacaccgtcttacccgaacccgtcggaccggttaccagcaccatcccgtaaggacggtgaatcgcttccaacaacaattttttctggaacggctcaaaaccgagctggtcgatgttcaaagacgcggcatcggaattcaaaatccgcatcacgaccttttcgccaaacagcgtcggcaatgtgctgacacggaaatcgacaggcttgccgcccttttgaaaggtcagctgcatcctaccgtcctgcggtatccgtttttcggaaatgtccaaacgcgacattaccttaatccgggaagcaagctgcccccttaccgcaatgggcggctgaaccacctcgcggagctgcccgtccacacggaaacggatacgcgcattgtgttcgtaaaactcgaaatggatgtcggatgccccgctacgcaaggcatccgacaaagttttatggataaacctcggaacagggccgtcttctgcctcctcgttgtcgatatacagggtgtggctttcctcttcctcttgcccctccccaagctcctgaagcagcgatgtcgaacgcgaacccacccaatcgagcaaacccgccaactggtcatcctcgacaatgaccaactcaaccgcaatccctgcggcagaaacggttttctgaatttgcggcatctgggtcggatcggaaaccgcaaaaaatactttgtcgccccgacggaaaaccggcacacagtggaactccaccatctgctcctccgtcaacacccccatcagcaccctgtggcgcggataatgacgcaaatcaagaatcgaataactgaacaccctcgcaatcaatgccgcaagcgacttgggcgaaatgacaccgtctgaaaacagcatcggcaacacttccttacccgcctgcgactcattgtagtaatgctcggcccgctcaacagtaaccacctggttttgaaccagaatcctcagcaaacctacgctcat

SEQ ID NO: 87 - RNA, 1677 nt, Neisseria gonorrhoeae
augagcguagguuugcugaggauucugguucaaaaccaggugguuacuguugagcgggccgagcauuacuacaaugagucgcaggcggguaaggaaguguugccgaugcuguuuucagacggugucauuucgcccaagucgcuugcggcauugauugcgaggguguucaguuauucgauucuugauuugcgucauuauccgcgccacagggugcugauggggguguugacggaggagcagaugguggaguuccacugugugccgguuuuccgucggggcgacaaaguauuuuuugcgguuuccgauccgacccagaugccgcaaauucagaaaaccguuucugccgcagggauugcgguugaguuggucauugucgaggaugaccaguuggcggguuugcucgauuggguggguucgcguucgacaucgcugcuucaggagcuuggggaggggcaagaggaagaggaaagccacacccuguauaucgacaacgaggaggcagaagacggcccuguuccgagguuuauccauaaaacuuugucggaugccuugcguagcggggcauccgacauccauuucgaguuuuacgaacacaaugcgcguauccguuuccguguggacgggcagcuccgcgaggugguucagccgcccauugcgguaagggggcagcuugcuucccggauuaagguaaugucgcguuuggacauuuccgaaaaacggauaccgcaggacgguaggaugcagcugaccuuucaaaagggcggcaagccugucgauuuccgugucagcacauugccgacgcuguuuggcgaaaaggucgugaugcggauuuugaauuccgaugccgcgucuuugaacaucgaccagcucgguuuugagccguuccagaaaaaauuguuguuggaagcgauucaccguccuuacgggauggugcugguaaccgguccgacggguucggguaagacggugucgcucuauaccuguuugaauauuuugaauacggagucgguaaauauugcaacggcggaagacccugccgagauuaaccugccgggcaucaaucagguuaacgucaaugauaagcagggucugacuuuugccgcugcuuugaagucuuuccugcgucaggacccggacaucauuauggucggugagauucgugauuuggaaacugccgauauugcgauuaaggcggcacaaacagggcauaugguguuuuccacacugcacacgaauaaugcgccggcgacguugucgcguaugcugaauaugggugucgcgccguuuaauauugccaguucggucagccugauuauggcgcagcgucuuuuacgcaggcuguguucgagcugcaaacaggaaguggaacgcccgucugccucugcuuugaaggaagucgguuucaccgaugaggaucuugcaaaagauuggaaacuuuaccgcgccgucgguugcgaccguugccgggggcaggguuauaaggggcgugcgggcguguaugagguuaugcccaucagcgaagaaaugcagcgugugauuaugaacaacgguacggaaguggguauuuuggacguugccuauaaggaggguaugguggauuugcgccgggccgguauuuugaaaauuaugcagggcauuacuucauuggaagagguaacggcaaauaccaacgauuag

SEQ ID NO: 88 - DNA, 309 bp, Neisseria gonorrhoeae
atgtacgcggtcgtaaaaaccggcggcaaacagtataaagtttccgtcggcgaaaaattgaaagtagaacagataccagcccaactcgacagccaaatcgaactgaccgaagttttgatgattgctgacggcgaatctgtaaaagttggcgcaccctttatcgaaggtgcaaaagtaacggctaaagtagtggcacacggtcgtggcgaaaaagtccgcatcttcaaaatgcgccgccgcaaacactaccaaaaacgccaaggccaccgccaaaatttcacccaaatcgaaatcgtggcaatcgcctaa

SEQ ID NO: 89 - DNA, 309 bp, Neisseria gonorrhoeae
ttaggcgattgccacgatttcgatttgggtgaaattttggcggtggccttggcgtttttggtagtgtttgcggcggcgcattttgaagatgcggactttttcgccacgaccgtgtgccactactttagccgttacttttgcaccttcgataaagggtgcgccaacttttacagattcgccgtcagcaatcatcaaaacttcggtcagttcgatttggctgtcgagttgggctggtatctgttctactttcaatttttcgccgacggaaactttatactgtttgccgccggtttttacgaccgcgtacat

SEQ ID NO: 90 - RNA, 309 nt, Neisseria gonorrhoeae
auguacgcggucguaaaaaccggcggcaaacaguauaaaguuuccgucggcgaaaaauugaaaguagaacagauaccagcccaacucgacagccaaaucgaacugaccgaaguuuugaugauugcugacggcgaaucuguaaaaguuggcgcacccuuuaucgaaggugcaaaaguaacggcuaaaguaguggcacacggucguggcgaaaaaguccgcaucuucaaaaugcgccgccgcaaacacuaccaaaaacgccaaggccaccgccaaaauuucacccaaaucgaaaucguggcaaucgccuaa

SEQ ID NO: 91 - DNA, 273 bp, Neisseria gonorrhoeae
atggcaagtaaaaaagcaggcggcagcacccgcaacggtcgcgattcagaagccaaacgcttgggcgttaaagcctacggcaacgagctgattcccgcaggttccatcatcgtacgccaacgcggtaccaaattccacgcaggcgacaacgtaggtatgggcaaagaccacactttgttcgccaaaattgacggttatgtcgaattcaaaaccaaaggcgcgctgaaccgtaaaactgtcagcatccgtccttacaccggttctgaagaataa

SEQ ID NO: 92 - DNA, 273 bp, Neisseria gonorrhoeae
ttattcttcagaaccggtgtaaggacggatgctgacagttttacggttcagcgcgcctttggttttgaattcgacataaccgtcaattttggcgaacaaagtgtggtctttgcccatacctacgttgtcgcctgcgtggaatttggtaccgcgttggcgtacgatgatggaacctgcgggaatcagctcgttgccgtaggctttaacgcccaagcgtttggcttctgaatcgcgaccgttgcgggtgctgccgcctgcttttttacttgccat

SEQ ID NO: 93 - RNA, 273 nt, Neisseria gonorrhoeae
auggcaaguaaaaaagcaggcggcagcacccgcaacggucgcgauucagaagccaaacgcuugggcguuaaagccuacggcaacgagcugauucccgcagguuccaucaucguacgccaacgcgguaccaaauuccacgcaggcgacaacguagguaugggcaaagaccacacuuuguucgccaaaauugacgguuaugucgaauucaaaaccaaaggcgcgcugaaccguaaaacugucagcauccguccuuacaccgguucugaagaauaa

SEQ ID NO: 94 - DNA, 156 bp, Neisseria gonorrhoeae
atgcgcgataaaatcaaactggaatccggtgcaggtactggccacttctacaccactaccaaaaataaacgcactatgcccggcaaactggaaatcaaaaaattcgatccggttgcccgcaaacacgtagtgtacaaagaaaccaaactgaaataa

SEQ ID NO: 95 - DNA, 156 bp, Neisseria gonorrhoeae
ttatttcagtttggtttctttgtacactacgtgtttgcgggcaaccggatcgaattttttgatttccagtttgccgggcatagtgcgtttatttttggtagtggtgtagaagtggccagtacctgcaccggattccagtttgattttatcgcgcat

SEQ ID NO: 96 - RNA, 156 nt, Neisseria gonorrhoeae
augcgcgauaaaaucaaacuggaauccggugcagguacuggccacuucuacaccacuaccaaaaauaaacgcacuaugcccggcaaacuggaaaucaaaaaauucgauccgguugcccgcaaacacguaguguacaaagaaaccaaacugaaauaa

SEQ ID NO: 97 - DNA, 450 bp, Neisseria gonorrhoeae
atggacgtacaactccccatcgaagccaaagacatccaaaaactcatcccccaccgctacccgtttctccagctcgaccgcattaccgccttcgagccgatgaaaaccctgaccgccatcaaaaacgtaaccataaacgaaccccaattccaaggccatttccccgacctgcccgttatgcccggcgtactcatcatcgaagcgatggcgcaggcgtgcggcacgttggcgattttgagcgaaggcgggcgcaaggaaaacgaatttttcttcttcgccggcatagacgaagcccgtttcaaacgccaagtcatccccggcgaccaactcgtctttgaagtcgaactcctgaccagccggcgcggcatcggcaaattcaacgccgttgccaaagtggacggacaagtcgccgtcgaagccgtgattatgtgcgccaaacgcgtggtttga

SEQ ID NO: 98 - DNA, 450 bp, Neisseria gonorrhoeae
tcaaaccacgcgtttggcgcacataatcacggcttcgacggcgacttgtccgtccactttggcaacggcgttgaatttgccgatgccgcgccggctggtcaggagttcgacttcaaagacgagttggtcgccggggatgacttggcgtttgaaacgggcttcgtctatgccggcgaagaagaaaaattcgttttccttgcgcccgccttcgctcaaaatcgccaacgtgccgcacgcctgcgccatcgcttcgatgatgagtacgccgggcataacgggcaggtcggggaaatggccttggaattggggttcgtttatggttacgtttttgatggcggtcagggttttcatcggctcgaaggcggtaatgcggtcgagctggagaaacgggtagcggtgggggatgagtttttggatgtctttggcttcgatggggagttgtacgtccat

SEQ ID NO: 99 - RNA, 450 nt, Neisseria gonorrhoeae
auggacguacaacuccccaucgaagccaaagacauccaaaaacucaucccccaccgcuacccguuucuccagcucgaccgcauuaccgccuucgagccgaugaaaacccugaccgccaucaaaaacguaaccauaaacgaaccccaauuccaaggccauuuccccgaccugcccguuaugcccggcguacucaucaucgaagcgauggcgcaggcgugcggcacguuggcgauuuugagcgaaggcgggcgcaaggaaaacgaauuuuucuucuucgccggcauagacgaagcccguuucaaacgccaagucauccccggcgaccaacucgucuuugaagucgaacuccugaccagccggcgcggcaucggcaaauucaacgccguugccaaaguggacggacaagucgccgucgaagccgugauuaugugcgccaaacgcgugguuuga

SEQ ID NO: 100 - DNA, 330 bp, Neisseria gonorrhoeae
atgagagtaaatgcacaacataaaaatgcccgtatctctgctcaaaaggctcgtttggtagctgatttgattcgtggtaaagacgttgcccaagctttgaatattttggctttcagccctaaaaaaggtgccgagctgatcaaaaaagtattggagtcagctattgctaatgctgagcataataacggtgcggacattgatgaactgaaagtggtaactatctttgttgacaaaggtccaagcttgaaacgttttcaagctcgcgccaaaggtcgcggtaaccgcatcgaaaaacaaacttgtcatatcaatgtgacagtgggtaactaa

SEQ ID NO: 101 - DNA, 330 bp, Neisseria gonorrhoeae
ttagttacccactgtcacattgatatgacaagtttgtttttcgatgcggttaccgcgacctttggcgcgagcttgaaaacgtttcaagcttggacctttgtcaacaaagatagttaccactttcagttcatcaatgtccgcaccgttattatgctcagcattagcaatagctgactccaatacttttttgatcagctcggcaccttttttagggctgaaagccaaaatattcaaagcttgggcaacgtctttaccacgaatcaaatcagctaccaaacgagccttttgagcagagatacgggcatttttatgttgtgcatttactctcat

SEQ ID NO: 102 - RNA, 330 nt, Neisseria gonorrhoeae
augagaguaaaugcacaacauaaaaaugcccguaucucugcucaaaaggcucguuugguagcugauuugauucgugguaaagacguugcccaagcuuugaauauuuuggcuuucagcccuaaaaaaggugccgagcugaucaaaaaaguauuggagucagcuauugcuaaugcugagcauaauaacggugcggacauugaugaacugaaagugguaacuaucuuuguugacaaagguccaagcuugaaacguuuucaagcucgcgccaaaggucgcgguaaccgcaucgaaaaacaaacuugucauaucaaugugacaguggguaacuaa

SEQ ID NO: 103 - DNA, 279 bp, Neisseria gonorrhoeae
atggctcgttcattgaaaaaaggcccatatgtagacctgcatttgctgaaaaaagtagatgctgttcgcgcaagcaacgacaaacgcccgattaaaacctggtctcgtcgttctaccattctgcctgattttatcggtctgaccattgccgtgcacaacggtcgcacccatgtgcctgtgtttatcagcgacaatatggttggtcataaattaggcgaattctcattgacccgtacctttaaaggccacctggccgataaaaaggctaaaaagaaataa

SEQ ID NO: 104 - DNA, 279 bp, Neisseria gonorrhoeae
ttatttctttttagcctttttatcggccaggtggcctttaaaggtacgggtcaatgagaattcgcctaatttatgaccaaccatattgtcgctgataaacacaggcacatgggtgcgaccgttgtgcacggcaatggtcagaccgataaaatcaggcagaatggtagaacgacgagaccaggttttaatcgggcgtttgtcgttgcttgcgcgaacagcatctacttttttcagcaaatgcaggtctacatatgggccttttttcaatgaacgagccat

SEQ ID NO: 105 - RNA, 279 nt, Neisseria gonorrhoeae
auggcucguucauugaaaaaaggcccauauguagaccugcauuugcugaaaaaaguagaugcuguucgcgcaagcaacgacaaacgcccgauuaaaaccuggucucgucguucuaccauucugccugauuuuaucggucugaccauugccgugcacaacggucgcacccaugugccuguguuuaucagcgacaauaugguuggucauaaauuaggcgaauucucauugacccguaccuuuaaaggccaccuggccgauaaaaaggcuaaaaagaaauaa

SEQ ID NO: 106 - DNA, 834 bp, Neisseria gonorrhoeae
atggcaatcgttaaaatgaagccgacctctgcaggccgtcgcggcatggttcgcgtggtaacagaaggtttgcacaaaggtgcaccttatgcacccttgcttgaaaagaaaaattctactgccggccgtaacaataatggtcatatcaccacccgtcacaaaggcggcggtcataaacaccattaccgtgttgtagactttaaacgtaacaaagacggcatttctgctaaagtagagcgtatcgaatacgatcctaaccgtactgccttcatcgcactgttgtgctatgcagacggcgagcgtcgttacatcatcgctcctcgcggtattcaagccggtgtcgtattggtttccggtgctgaagctgccatcaaagtaggcaacaccctgccgatccgcaacattcccgttggtacgactatccactgtatcgaaatgaaacccggtaaaggtgctcaaatcgcacgttctgccggtgcttctgcggtattgttggctaaagaaggtgcatacgctcaagtccgtctgcgctctggcgaagttcgtaaaatcaacgtagattgccgtgcgaccatcggtgaagtcggtaacgaagagcaaagcctgaaaaaaatcggtaaagccggtgctaaccgttggcgcggtattcgtccgaccgtacgcggtgttgtcatgaatcccgtcgatcacccgcatggtggtggtgaaggccgtaccggcgaagcccgcgaaccggttagtccatggggtactcctgctaaaggctaccgcactcgtaataacaaacgcacggataatatgattgttcgtcgccgttactcaaataaaggttaa

SEQ ID NO: 107 - DNA, 834 bp, Neisseria gonorrhoeae
ttaacctttatttgagtaacggcgacgaacaatcatattatccgtgcgtttgttattacgagtgcggtagcctttagcaggagtaccccatggactaaccggttcgcgggcttcgccggtacggccttcaccaccaccatgcgggtgatcgacgggattcatgacaacaccgcgtacggtcggacgaataccgcgccaacggttagcaccggctttaccgatttttttcaggctttgctcttcgttaccgacttcaccgatggtcgcacggcaatctacgttgattttacgaacttcgccagagcgcagacggacttgagcgtatgcaccttctttagccaacaataccgcagaagcaccggcagaacgtgcgatttgagcacctttaccgggtttcatttcgatacagtggatagtcgtaccaacgggaatgttgcggatcggcagggtgttgcctactttgatggcagcttcagcaccggaaaccaatacgacaccggcttgaataccgcgaggagcgatgatgtaacgacgctcgccgtctgcatagcacaacagtgcgatgaaggcagtacggttaggatcgtattcgatacgctctactttagcagaaatgccgtctttgttacgtttaaagtctacaacacggtaatggtgtttatgaccgccgcctttgtgacgggtggtgatatgaccattattgttacggccggcagtagaatttttcttttcaagcaagggtgcataaggtgcacctttgtgcaaaccttctgttaccacgcgaaccatgccgcgacggcctgcagaggtcggcttcattttaacgattgccat

SEQ ID NO: 108 - RNA, 834 nt, Neisseria gonorrhoeae
auggcaaucguuaaaaugaagccgaccucugcaggccgucgcggcaugguucgcgugguaacagaagguuugcacaaaggugcaccuuaugcacccuugcuugaaaagaaaaauucuacugccggccguaacaauaauggucauaucaccacccgucacaaaggcggcggucauaaacaccauuaccguguuguagacuuuaaacguaacaaagacggcauuucugcuaaaguagagcguaucgaauacgauccuaaccguacugccuucaucgcacuguugugcuaugcagacggcgagcgucguuacaucaucgcuccucgcgguauucaagccggugucguauugguuuccggugcugaagcugccaucaaaguaggcaacacccugccgauccgcaacauucccguugguacgacuauccacuguaucgaaaugaaacccgguaaaggugcucaaaucgcacguucugccggugcuucugcgguauuguuggcuaaagaaggugcauacgcucaaguccgucugcgcucuggcgaaguucguaaaaucaacguagauugccgugcgaccaucggugaagucgguaacgaagagcaaagccugaaaaaaaucgguaaagccggugcuaaccguuggcgcgguauucguccgaccguacgcgguguugucaugaaucccgucgaucacccgcauggugguggugaaggccguaccggcgaagcccgcgaaccgguuaguccaugggguacuccugcuaaaggcuaccgcacucguaauaacaaacgcacggauaauaugauuguucgucgccguuacucaaauaaagguuaa

SEQ ID NO: 109 - DNA, 621 bp, Neisseria gonorrhoeae
atggaattgaaagtaattgacgctaaaggacaagtttcaggcagcctgtctgtttctgatgctttgttcgcccgcgaatacaatgaagcgttggttcaccagctggtaaatgcctacttggcaaacgcccgctctggtaaccgtgctcaaaaaacccgtgccgaagtaaaacactcaaccaaaaaaccatggcgtcaaaaaggtaccggccgcgcccgttccggtatgacttcttctccgctgtggcgtaaaggcggtcgcgcgttcccgaacaaacccgacgaaaacttcactcaaaaagtaaaccgtaaaatgtaccgtgccggtatggcgactatcctgtcccaattggcgcgtgacgagcgtttgtttgtgattgaggcgttgactgccgaaactcccaaaaccaaagtttttgccgaacaagtaaaaaatttggctctggagcaagtgctgtttgtaaccaaacggctcgacgagaatgtttacttggcttcacgcaacttgccaaacgtattggttttggaagctcaacaagttgatccttacagcttgctgcgttataaaaaagtaatcatcactaaagatgcggttgcacaattagaggagcaatgggtatga

SEQ ID NO: 110 - DNA, 621 bp, Neisseria gonorrhoeae
tcatacccattgctcctctaattgtgcaaccgcatctttagtgatgattacttttttataacgcagcaagctgtaaggatcaacttgttgagcttccaaaaccaatacgtttggcaagttgcgtgaagccaagtaaacattctcgtcgagccgtttggttacaaacagcacttgctccagagccaaattttttacttgttcggcaaaaactttggttttgggagtttcggcagtcaacgcctcaatcacaaacaaacgctcgtcacgcgccaattgggacaggatagtcgccataccggcacggtacattttacggtttactttttgagtgaagttttcgtcgggtttgttcgggaacgcgcgaccgcctttacgccacagcggagaagaagtcataccggaacgggcgcggccggtacctttttgacgccatggttttttggttgagtgttttacttcggcacgggttttttgagcacggttaccagagcgggcgtttgccaagtaggcatttaccagctggtgaaccaacgcttcattgtattcgcgggcgaacaaagcatcagaaacagacaggctgcctgaaacttgtcctttagcgtcaattactttcaattccat

SEQ ID NO: 111 - RNA, 621 nt, Neisseria gonorrhoeae
auggaauugaaaguaauugacgcuaaaggacaaguuucaggcagccugucuguuucugaugcuuuguucgcccgcgaauacaaugaagcguugguucaccagcugguaaaugccuacuuggcaaacgcccgcucugguaaccgugcucaaaaaacccgugccgaaguaaaacacucaaccaaaaaaccauggcgucaaaaagguaccggccgcgcccguuccgguaugacuucuucuccgcuguggcguaaaggcggucgcgcguucccgaacaaacccgacgaaaacuucacucaaaaaguaaaccguaaaauguaccgugccgguauggcgacuauccugucccaauuggcgcgugacgagcguuuguuugugauugaggcguugacugccgaaacucccaaaaccaaaguuuuugccgaacaaguaaaaaauuuggcucuggagcaagugcuguuuguaaccaaacggcucgacgagaauguuuacuuggcuucacgcaacuugccaaacguauugguuuuggaagcucaacaaguugauccuuacagcuugcugcguuauaaaaaaguaaucaucacuaaagaugcgguugcacaauuagaggagcaauggguauga

SEQ ID NO: 112 - DNA, 2106 bp, Neisseria gonorrhoeae
atggctcgtaagaccccgatcagcctgtaccgcaacatcggtatttccgcccatatcgatgcgggtaaaaccacgacgacagaacgtattttgttctataccggtttgacccacaagctgggcgaagtgcatgacggtgcggctactaccgactacatggaacaagagcaagagcgcggtattaccattacctccgctgccgttacttcctactggtccggtatggcgaaacaattccccgagcaccgcttcaacatcatcgacaccccggggcacgttgactttaccgtagaggtagagcgttctatgcgtgtattggacggcgcggtaatggtttactgtgcggtgggcggtgttcaaccgcaatctgaaaccgtatggcggcaagccaacaaataccaagttccgcgcttggcgtttgtcaataaaatggaccgccaaggtgccaacttcttccgcgttgtcgagcaaatgaaaacccgtttgcgcgcaaaccccgtacctatcgtcattccggtaggcgcggaagacagttttaccggtgttgtcgatttgctgaaaatgaaatctatcatctggaatgaagccgataaaggtacaacctttacctatggcgatattcctgccgaattggtcgaaactgccgaagaatggcgtcaaaatatgattgaagccgcagccgaagccagcgaagaactgatggacaaatacttgggcggtgaagatctggccgaagaagaaatcgtaggcgcgttgcgtcaacgtactttggcaggcgaaattcagcctatgctgtgcggttctgcatttaaaaacaaaggtgttcaacgtatgttggacgcagttgtagaattgctgccagctcctaccgatattcctccggttcaaggtgttaatcctaacactgaagaagccgacagccgtcaagccagcgatgaagagaaattctctgcattggcattcaaaatgttgaacgacaaatacgtcggtcagctgacctttatccgcgtttactcaggcgtagtaaaatccggcgataccgtactgaattctgtaaaaggcactcgcgaacgtatcggtcgtttggtgcaaatgactgccgcagaccgtactgaaatcgaagaagtacgcgctggcgacatcgcagccgctatcggtctgaaagacgttactaccggtgaaaccttgtgtgcggaaagcgcgccgattatcttggaacgtatggaattccccgagccggtaatccatattgccgttgagccgaaaaccaaagccgaccaagagaaaatgggtatcgccctgaaccgcttggctaaagaagacccttctttccgcgttcgtacagacgaagaatccggtcaaaccattatttccggtatgggtgagctgcacttggaaattattgttgaccgtatgaaacgcgaattcggtgtggaagcaaatatcggtgcacctcaagtggcttaccgtgaaactatccgcaaagccgttaaagctgaatacaaacatgcaaaacaatccggtggtaaaggtcaatacggtcacgttgtgattgaaatggaacctatggaaccgggtggtgaaggttacgagtttatcgatgaaattaaaggtggtgtgattcctcgcgaatttattccgtctgtcgataaaggtatccgcgatacgttgcctaacggtatcgttgccggctatcctgtagttgacgtacgtatccgtctggtattcggttcttaccatgatgtcgactcttcccaattggcatttgaattggctgcttctcaagcgtttaaagaaggtatgcgtcaagcatctcctgccctgcttgagccgattatggcagttgaagtggaaactccggaagaatacatgggcgacgtaatgggcgacttgaaccgccgtcgcggtgttgtattgggtatggatgatgacggtatcggcggtaaaaaagtccgtgccgaagtacctctggcagaaatgttcggttactcgaccgacctgcgttctgcaacccaaggccgcgctacttactctatggagttcaagaaatattctgaagctcctgcccacatagctgctgctgtaactgaagcccgtaaaggctaa

SEQ ID NO: 113 - DNA, 2106 bp, Neisseria gonorrhoeae
ttagcctttacgggcttcagttacagcagcagctatgtgggcaggagcttcagaatatttcttgaactccatagagtaagtagcgcggccttgggttgcagaacgcaggtcggtcgagtaaccgaacatttctgccagaggtacttcggcacggacttttttaccgccgataccgtcatcatccatacccaatacaacaccgcgacggcggttcaagtcgcccattacgtcgcccatgtattcttccggagtttccacttcaactgccataatcggctcaagcagggcaggagatgcttgacgcataccttctttaaacgcttgagaagcagccaattcaaatgccaattgggaagagtcgacatcatggtaagaaccgaataccagacggatacgtacgtcaactacaggatagccggcaacgataccgttaggcaacgtatcgcggatacctttatcgacagacggaataaattcgcgaggaatcacaccacctttaatttcatcgataaactcgtaaccttcaccacccggttccataggttccatttcaatcacaacgtgaccgtattgacctttaccaccggattgttttgcatgtttgtattcagctttaacggctttgcggatagtttcacggtaagccacttgaggtgcaccgatatttgcttccacaccgaattcgcgtttcatacggtcaacaataatttccaagtgcagctcacccataccggaaataatggtttgaccggattcttcgtctgtacgaacgcggaaagaagggtcttctttagccaagcggttcagggcgatacccattttctcttggtcggctttggttttcggctcaacggcaatatggattaccggctcggggaattccatacgttccaagataatcggcgcgctttccgcacacaaggtttcaccggtagtaacgtctttcagaccgatagcggctgcgatgtcgccagcgcgtacttcttcgatttcagtacggtctgcggcagtcatttgcaccaaacgaccgatacgttcgcgagtgccttttacagaattcagtacggtatcgccggattttactacgcctgagtaaacgcggataaaggtcagctgaccgacgtatttgtcgttcaacattttgaatgccaatgcagagaatttctcttcatcgctggcttgacggctgtcggcttcttcagtgttaggattaacaccttgaaccggaggaatatcggtaggagctggcagcaattctacaactgcgtccaacatacgttgaacacctttgtttttaaatgcagaaccgcacagcataggctgaatttcgcctgccaaagtacgttgacgcaacgcgcctacgatttcttcttcggccagatcttcaccgcccaagtatttgtccatcagttcttcgctggcttcggctgcggcttcaatcatattttgacgccattcttcggcagtttcgaccaattcggcaggaatatcgccataggtaaaggttgtacctttatcggcttcattccagatgatagatttcattttcagcaaatcgacaacaccggtaaaactgtcttccgcgcctaccggaatgacgataggtacggggtttgcgcgcaaacgggttttcatttgctcgacaacgcggaagaagttggcaccttggcggtccattttattgacaaacgccaagcgcggaacttggtatttgttggcttgccgccatacggtttcagattgcggttgaacaccgcccaccgcacagtaaaccattaccgcgccgtccaatacacgcatagaacgctctacctctacggtaaagtcaacgtgccccggggtgtcgatgatgttgaagcggtgctcggggaattgtttcgccataccggaccagtaggaagtaacggcagcggaggtaatggtaataccgcgctcttgctcttgttccatgtagtcggtagtagccgcaccgtcatgcacttcgcccagcttgtgggtcaaaccggtatagaacaaaatacgttctgtcgtcgtggttttacccgcatcgatatgggcggaaataccgatgttgcggtacaggctgatcggggtcttacgagccat

SEQ ID NO: 114 - RNA, 2106 nt, Neisseria gonorrhoeae
auggcucguaagaccccgaucagccuguaccgcaacaucgguauuuccgcccauaucgaugcggguaaaaccacgacgacagaacguauuuuguucuauaccgguuugacccacaagcugggcgaagugcaugacggugcggcuacuaccgacuacauggaacaagagcaagagcgcgguauuaccauuaccuccgcugccguuacuuccuacugguccgguauggcgaaacaauuccccgagcaccgcuucaacaucaucgacaccccggggcacguugacuuuaccguagagguagagcguucuaugcguguauuggacggcgcgguaaugguuuacugugcggugggcgguguucaaccgcaaucugaaaccguauggcggcaagccaacaaauaccaaguuccgcgcuuggcguuugucaauaaaauggaccgccaaggugccaacuucuuccgcguugucgagcaaaugaaaacccguuugcgcgcaaaccccguaccuaucgucauuccgguaggcgcggaagacaguuuuaccgguguugucgauuugcugaaaaugaaaucuaucaucuggaaugaagccgauaaagguacaaccuuuaccuauggcgauauuccugccgaauuggucgaaacugccgaagaauggcgucaaaauaugauugaagccgcagccgaagccagcgaagaacugauggacaaauacuugggcggugaagaucuggccgaagaagaaaucguaggcgcguugcgucaacguacuuuggcaggcgaaauucagccuaugcugugcgguucugcauuuaaaaacaaagguguucaacguauguuggacgcaguuguagaauugcugccagcuccuaccgauauuccuccgguucaagguguuaauccuaacacugaagaagccgacagccgucaagccagcgaugaagagaaauucucugcauuggcauucaaaauguugaacgacaaauacgucggucagcugaccuuuauccgcguuuacucaggcguaguaaaauccggcgauaccguacugaauucuguaaaaggcacucgcgaacguaucggucguuuggugcaaaugacugccgcagaccguacugaaaucgaagaaguacgcgcuggcgacaucgcagccgcuaucggucugaaagacguuacuaccggugaaaccuugugugcggaaagcgcgccgauuaucuuggaacguauggaauuccccgagccgguaauccauauugccguugagccgaaaaccaaagccgaccaagagaaaauggguaucgcccugaaccgcuuggcuaaagaagacccuucuuuccgcguucguacagacgaagaauccggucaaaccauuauuuccgguaugggugagcugcacuuggaaauuauuguugaccguaugaaacgcgaauucgguguggaagcaaauaucggugcaccucaaguggcuuaccgugaaacuauccgcaaagccguuaaagcugaauacaaacaugcaaaacaauccggugguaaaggucaauacggucacguugugauugaaauggaaccuauggaaccggguggugaagguuacgaguuuaucgaugaaauuaaagguggugugauuccucgcgaauuuauuccgucugucgauaaagguauccgcgauacguugccuaacgguaucguugccggcuauccuguaguugacguacguauccgucugguauucgguucuuaccaugaugucgacucuucccaauuggcauuugaauuggcugcuucucaagcguuuaaagaagguaugcgucaagcaucuccugcccugcuugagccgauuauggcaguugaaguggaaacuccggaagaauacaugggcgacguaaugggcgacuugaaccgccgucgcgguguuguauuggguauggaugaugacgguaucggcgguaaaaaaguccgugccgaaguaccucuggcagaaauguucgguuacucgaccgaccugcguucugcaacccaaggccgcgcuacuuacucuauggaguucaagaaauauucugaagcuccugcccacauagcugcugcuguaacugaagcccguaaaggcuaa

SEQ ID NO: 115 - DNA, 471 bp, Neisseria gonorrhoeae
atgccaagacgtagagaagtccccaagcgcgacgtactgccagatcctaaattcggtagcgtcgagttgaccaaattcatgaacgtattgatgattgacggtaaaaaatccgttgccgagcgtatcgtttacggtgcgttggaacagattgagaaaaaaaccggcaaagcagcaatcgaagtatttaacgaagccattgcaaactccaaacctatcgtggaagtgaaaagccgccgtgtaggtggtgcaaactaccaagttcctgttgaagttcgtccttcacgccgtctggctttggcaatgcgttgggttcgcgacgcggcccgcaaacgtggtgagaaatccatggatctgcgtttggcaggcgagttgattgatgcgtccgaaggccgtggcggtgcgttgaaaaaacgtgaagaagtacaccgtatggctgaagccaacaaagcattctctcacttccgtttctaa

SEQ ID NO: 116 - DNA, 471 bp, Neisseria gonorrhoeae
ttagaaacggaagtgagagaatgctttgttggcttcagccatacggtgtacttcttcacgttttttcaacgcaccgccacggccttcggacgcatcaatcaactcgcctgccaaacgcagatccatggatttctcaccacgtttgcgggccgcgtcgcgaacccaacgcattgccaaagccagacggcgtgaaggacgaacttcaacaggaacttggtagtttgcaccacctacacggcggcttttcacttccacgataggtttggagtttgcaatggcttcgttaaatacttcgattgctgctttgccggtttttttctcaatctgttccaacgcaccgtaaacgatacgctcggcaacggattttttaccgtcaatcatcaatacgttcatgaatttggtcaactcgacgctaccgaatttaggatctggcagtacgtcgcgcttggggacttctctacgtcttggcat

SEQ ID NO: 117 - RNA, 471 nt, Neisseria gonorrhoeae
augccaagacguagagaaguccccaagcgcgacguacugccagauccuaaauucgguagcgucgaguugaccaaauucaugaacguauugaugauugacgguaaaaaauccguugccgagcguaucguuuacggugcguuggaacagauugagaaaaaaaccggcaaagcagcaaucgaaguauuuaacgaagccauugcaaacuccaaaccuaucguggaagugaaaagccgccguguagguggugcaaacuaccaaguuccuguugaaguucguccuucacgccgucuggcuuuggcaaugcguuggguucgcgacgcggcccgcaaacguggugagaaauccauggaucugcguuuggcaggcgaguugauugaugcguccgaaggccguggcggugcguugaaaaaacgugaagaaguacaccguauggcugaagccaacaaagcauucucucacuuccguuucuaa

SEQ ID NO: 118 - DNA, 372 bp, Neisseria gonorrhoeae
atgccaactatcaaccaattggtacgcaaaggccgtcaaaagcccgtgtacgtaaacaaagtgcccgcactggaagcctgcccgcaaaaacgcggcgtgtgcacccgtgtatacacgactacccctagaaaacctaactctgcattgcgtaaagtatgtaaagtccgcctgaccaacggttttgaagtcatttcatatatcggcggtgaaggccacaacctgcaagagcacagcgtcgtactgattcgcggcggccgtgtaaaagacttgccgggtgtacgttaccacactgtacgcggttctttggatactgcaggtgttaaagaccgcaaacaagcccgttctaaatacggtgctaagcgtcctaaataa

SEQ ID NO: 119 - DNA, 372 bp, Neisseria gonorrhoeae
ttatttaggacgcttagcaccgtatttagaacgggcttgtttgcggtctttaacacctgcagtatccaaagaaccgcgtacagtgtggtaacgtacacccggcaagtcttttacacggccgccgcgaatcagtacgacgctgtgctcttgcaggttgtggccttcaccgccgatatatgaaatgacttcaaaaccgttggtcaggcggactttacatactttacgcaatgcagagttaggttttctaggggtagtcgtgtatacacgggtgcacacgccgcgtttttgcgggcaggcttccagtgcgggcactttgtttacgtacacgggcttttgacggcctttgcgtaccaattggttgatagttggcat

SEQ ID NO: 120 - RNA, 372 nt, Neisseria gonorrhoeae
augccaacuaucaaccaauugguacgcaaaggccgucaaaagcccguguacguaaacaaagugcccgcacuggaagccugcccgcaaaaacgcggcgugugcacccguguauacacgacuaccccuagaaaaccuaacucugcauugcguaaaguauguaaaguccgccugaccaacgguuuugaagucauuucauauaucggcggugaaggccacaaccugcaagagcacagcgucguacugauucgcggcggccguguaaaagacuugccggguguacguuaccacacuguacgcgguucuuuggauacugcagguguuaaagaccgcaaacaagcccguucuaaauacggugcuaagcguccuaaauaa

SEQ ID NO: 121 - DNA, 1215 bp, Neisseria gonorrhoeae
atggaatgggcgtttaacagttattacaccttgattgccgccactttggttttgttggtcggcaaggttttggttaagaaaatcaaaatcttgcgtgattttaacatccccgaacccgtggcgggcgggctgattgccgcgattatcctgtttgcgctgcacgaggcgtacggcgtgagcttcaaatttgagaaaccgctgcaaaatgcgtttatgctgattttcttcacgtccatcggcttgagcgcggatttttcccgtttgaaggcgggcggtttgccgctggtggtttttaccgcgattgtgggcggatttatcttggtgcaaaactttgtcggggtcggactggctacggctttgggtttggacccgctcatcggtctgattaccggttcggtgtcgctgacgggcggacacggcacgtcaggtgcgtggggacctaattttgaaacgcaatacggcttggtcggcgcaaccggtttgggtattgcttcggttactttcgggctggtgttcggcggcctgatcggagggccggttgcgcgccgcctgatcaacaaaatgggccgcaaaccggttgaaaacacaaaacaggatcaggacgacaacgcggacgacgtgttcgagcaggcaaaacgcacccgcctgattacggcggaatctgccgttgaaacgcttgccatgtttgccgcgtgtctggcgtttgccgagattatggacggtttcgacaaagaatacctgttcgacctgcccaaattcgtgtggtgtctgtttggcggcgtggttatccgcaacatccttaccgccgcattcaaggtcaatatgttcgaccgtgccatcgatgtgttcggcaatgcttcgctttcgcttttcttggcaatggcgttgctgaatttgaaactgtgggagctgaccggtttggcggggtctgtaaccgtgattcttgcagtacaaaccgcagtgatggttttgtacgcgacttttgttacctatgtctttatggggcgcgactatgatgccgcagtattggctgccggccactgcggtttcggtttgggcgcaacgccgacggcggtggcaaatatgcagtccgtcacgcatactttcggcgcgtcacataaggcgtttttgattgtgcctatggtcggcgcgttctttgtcgatttgattaatgccgcgattctcaccggttttgtgaatttctttaaaggctga

SEQ ID NO: 122 - DNA, 1215 bp, Neisseria gonorrhoeae
tcagcctttaaagaaattcacaaaaccggtgagaatcgcggcattaatcaaatcgacaaagaacgcgccgaccataggcacaatcaaaaacgccttatgtgacgcgccgaaagtatgcgtgacggactgcatatttgccaccgccgtcggcgttgcgcccaaaccgaaaccgcagtggccggcagccaatactgcggcatcatagtcgcgccccataaagacataggtaacaaaagtcgcgtacaaaaccatcactgcggtttgtactgcaagaatcacggttacagaccccgccaaaccggtcagctcccacagtttcaaattcagcaacgccattgccaagaaaagcgaaagcgaagcattgccgaacacatcgatggcacggtcgaacatattgaccttgaatgcggcggtaaggatgttgcggataaccacgccgccaaacagacaccacacgaatttgggcaggtcgaacaggtattctttgtcgaaaccgtccataatctcggcaaacgccagacacgcggcaaacatggcaagcgtttcaacggcagattccgccgtaatcaggcgggtgcgttttgcctgctcgaacacgtcgtccgcgttgtcgtcctgatcctgttttgtgttttcaaccggtttgcggcccattttgttgatcaggcggcgcgcaaccggccctccgatcaggccgccgaacaccagcccgaaagtaaccgaagcaatacccaaaccggttgcgccgaccaagccgtattgcgtttcaaaattaggtccccacgcacctgacgtgccgtgtccgcccgtcagcgacaccgaaccggtaatcagaccgatgagcgggtccaaacccaaagccgtagccagtccgaccccgacaaagttttgcaccaagataaatccgcccacaatcgcggtaaaaaccaccagcggcaaaccgcccgccttcaaacgggaaaaatccgcgctcaagccgatggacgtgaagaaaatcagcataaacgcattttgcagcggtttctcaaatttgaagctcacgccgtacgcctcgtgcagcgcaaacaggataatcgcggcaatcagcccgcccgccacgggttcggggatgttaaaatcacgcaagattttgattttcttaaccaaaaccttgccgaccaacaaaaccaaagtggcggcaatcaaggtgtaataactgttaaacgcccattccat

SEQ ID NO: 123 - RNA, 1215 nt, Neisseria gonorrhoeae
auggaaugggcguuuaacaguuauuacaccuugauugccgccacuuugguuuuguuggucggcaagguuuugguuaagaaaaucaaaaucuugcgugauuuuaacauccccgaacccguggcgggcgggcugauugccgcgauuauccuguuugcgcugcacgaggcguacggcgugagcuucaaauuugagaaaccgcugcaaaaugcguuuaugcugauuuucuucacguccaucggcuugagcgcggauuuuucccguuugaaggcgggcgguuugccgcuggugguuuuuaccgcgauugugggcggauuuaucuuggugcaaaacuuugucggggucggacuggcuacggcuuuggguuuggacccgcucaucggucugauuaccgguucggugucgcugacgggcggacacggcacgucaggugcguggggaccuaauuuugaaacgcaauacggcuuggucggcgcaaccgguuuggguauugcuucgguuacuuucgggcugguguucggcggccugaucggagggccgguugcgcgccgccugaucaacaaaaugggccgcaaaccgguugaaaacacaaaacaggaucaggacgacaacgcggacgacguguucgagcaggcaaaacgcacccgccugauuacggcggaaucugccguugaaacgcuugccauguuugccgcgugucuggcguuugccgagauuauggacgguuucgacaaagaauaccuguucgaccugcccaaauucguguggugucuguuuggcggcgugguuauccgcaacauccuuaccgccgcauucaaggucaauauguucgaccgugccaucgauguguucggcaaugcuucgcuuucgcuuuucuuggcaauggcguugcugaauuugaaacugugggagcugaccgguuuggcggggucuguaaccgugauucuugcaguacaaaccgcagugaugguuuuguacgcgacuuuuguuaccuaugucuuuauggggcgcgacuaugaugccgcaguauuggcugccggccacugcgguuucgguuugggcgcaacgccgacggcgguggcaaauaugcaguccgucacgcauacuuucggcgcgucacauaaggcguuuuugauugugccuauggucggcgcguucuuugucgauuugauuaaugccgcgauucucaccgguuuugugaauuucuuuaaaggcuga

SEQ ID NO: 124 - DNA, 432 bp, Neisseria gonorrhoeae
atgaaaaccttttcagcgaaaccccacgaggtgaagcgcgaatggttcgtcatcgatgcccaagacaaagtcttgggtcgcgttgcaaccgaagtcgccagccgtctgcgtggcaaacacaaacctgaatacaccccccacgtcgataccggcgattacatcatcgtcatcaatgcggacaaactgcgtgtaaccggtgccaaattcgaagataaaaaatacttccgccattccggttttccaggcggcatctacgagcgcactttccgcgaaatgcaagatcaattcccgggccgcgctttggagcaggctgtaaaaggtatgctgcccaaaggtccgctgggttacgccatgattaaaaaactgaaagtgtacgctggtgcggagcatgcccatgctgcgcaacaacccaaagttttggaactgaaataa

SEQ ID NO: 125 - DNA, 432 bp, Neisseria gonorrhoeae
ttatttcagttccaaaactttgggttgttgcgcagcatgggcatgctccgcaccagcgtacactttcagttttttaatcatggcgtaacccagcggacctttgggcagcataccttttacagcctgctccaaagcgcggcccgggaattgatcttgcatttcgcggaaagtgcgctcgtagatgccgcctggaaaaccggaatggcggaagtattttttatcttcgaatttggcaccggttacacgcagtttgtccgcattgatgacgatgatgtaatcgccggtatcgacgtggggggtgtattcaggtttgtgtttgccacgcagacggctggcgacttcggttgcaacgcgacccaagactttgtcttgggcatcgatgacgaaccattcgcgcttcacctcgtggggtttcgctgaaaaggttttcat

SEQ ID NO: 126 - RNA, 432 nt, Neisseria gonorrhoeae
augaaaaccuuuucagcgaaaccccacgaggugaagcgcgaaugguucgucaucgaugcccaagacaaagucuugggucgcguugcaaccgaagucgccagccgucugcguggcaaacacaaaccugaauacaccccccacgucgauaccggcgauuacaucaucgucaucaaugcggacaaacugcguguaaccggugccaaauucgaagauaaaaaauacuuccgccauuccgguuuuccaggcggcaucuacgagcgcacuuuccgcgaaaugcaagaucaauucccgggccgcgcuuuggagcaggcuguaaaagguaugcugcccaaagguccgcuggguuacgccaugauuaaaaaacugaaaguguacgcuggugcggagcaugcccaugcugcgcaacaacccaaaguuuuggaacugaaauaa

SEQ ID NO: 127 - DNA, 1221 bp, Neisseria gonorrhoeae
atgaccctgttttgcgaacaagtcccctacccccgccttgccgaagaattcggcacgccgctttatgtgtacagccaatccgcgctgaccggagcatttgaaaactatcaaaccgcctttgccgctttgaacccgcttgtctgctacgccgtcaaggcaaacggcaacctgagcattatcaaacactttgcttctttgggcagcggttttgacattgtgtcgggcggcgaattggcacgcgttttggcggcaggcggcgatgcggcgaaaacgattttttccggcgtaggcaaaagcgaggcggaaatcgagttcgcgctgaatgccggcgtaaaatgcttcaatatggaaagcatccccgaaatcgaccgcattcagaaaattgccgcgcgtttgggcaaaaccgcgcccgtctccctgcgcgtcaatcccgatgtcgatgcaaaaacccatccctacatctccacaggtctgaaagccaacaaattcggcatcgcctacgccgacgcgctcgaagcctaccgccatgccgcacaacagcccaatttgaaaatcatcggcatcgactgccacatcggttcgcaactgaccgacttaagcccactggtcgaagcctgcgaacgcattttgattttggttgacgctcttgccgccgaaggcattgttttggaacatttggacttaggcggcggcgtcggcattgtttacaaagacgaaggcgtccccgatttgggtgcgtatgcccgagcggttcaaaaactgatggggacacgccgtctgaaactcattcttgagccaggccgcagcttggtcggcaacgcaggtgcattgctgacgcgcgtcgaatttgtcaaacacggtgaagagaaaaactttgtgatggtcgatgcggcgatgaacgatttgatgcgcccagccctatacgatgcctaccaccacatcgaagcggttgaaaccaaaaacattgagcctctgaccgccaacatcgtcggcccgatttgtgaaaccggcgacttcctcggcaaagaccgcaccatcgcctgcgaagaaggcgatttgctgcttatccgcagcgcgggcgcatacggggccagtatggctagcaattacaacacgcgcaaccgtgcggcggaggtgttggttgacggcggcggatacaaactcatccgccggcgcgaaaccttggaacagcaaatggcaaacgaactcgcctgcctataa

SEQ ID NO: 128 - DNA, 1221 bp, Neisseria gonorrhoeae
ttataggcaggcgagttcgtttgccatttgctgttccaaggtttcgcgccggcggatgagtttgtatccgccgccgtcaaccaacacctccgccgcacggttgcgcgtgttgtaattgctagccatactggccccgtatgcgcccgcgctgcggataagcagcaaatcgccttcttcgcaggcgatggtgcggtctttgccgaggaagtcgccggtttcacaaatcgggccgacgatgttggcggtcagaggctcaatgtttttggtttcaaccgcttcgatgtggtggtaggcatcgtatagggctgggcgcatcaaatcgttcatcgccgcatcgaccatcacaaagtttttctcttcaccgtgtttgacaaattcgacgcgcgtcagcaatgcacctgcgttgccgaccaagctgcggcctggctcaagaatgagtttcagacggcgtgtccccatcagtttttgaaccgctcgggcatacgcacccaaatcggggacgccttcgtctttgtaaacaatgccgacgccgccgcctaagtccaaatgttccaaaacaatgccttcggcggcaagagcgtcaaccaaaatcaaaatgcgttcgcaggcttcgaccagtgggcttaagtcggtcagttgcgaaccgatgtggcagtcgatgccgatgattttcaaattgggctgttgtgcggcatggcggtaggcttcgagcgcgtcggcgtaggcgatgccgaatttgttggctttcagacctgtggagatgtagggatgggtttttgcatcgacatcgggattgacgcgcagggagacgggcgcggttttgcccaaacgcgcggcaattttctgaatgcggtcgatttcggggatgctttccatattgaagcattttacgccggcattcagcgcgaactcgatttccgcctcgcttttgcctacgccggaaaaaatcgttttcgccgcatcgccgcctgccgccaaaacgcgtgccaattcgccgcccgacacaatgtcaaaaccgctgcccaaagaagcaaagtgtttgataatgctcaggttgccgtttgccttgacggcgtagcagacaagcgggttcaaagcggcaaaggcggtttgatagttttcaaatgctccggtcagcgcggattggctgtacacataaagcggcgtgccgaattcttcggcaaggcgggggtaggggacttgttcgcaaaacagggtcat

SEQ ID NO: 129 - RNA, 1221 nt, Neisseria gonorrhoeae
augacccuguuuugcgaacaaguccccuacccccgccuugccgaagaauucggcacgccgcuuuauguguacagccaauccgcgcugaccggagcauuugaaaacuaucaaaccgccuuugccgcuuugaacccgcuugucugcuacgccgucaaggcaaacggcaaccugagcauuaucaaacacuuugcuucuuugggcagcgguuuugacauugugucgggcggcgaauuggcacgcguuuuggcggcaggcggcgaugcggcgaaaacgauuuuuuccggcguaggcaaaagcgaggcggaaaucgaguucgcgcugaaugccggcguaaaaugcuucaauauggaaagcauccccgaaaucgaccgcauucagaaaauugccgcgcguuugggcaaaaccgcgcccgucucccugcgcgucaaucccgaugucgaugcaaaaacccaucccuacaucuccacaggucugaaagccaacaaauucggcaucgccuacgccgacgcgcucgaagccuaccgccaugccgcacaacagcccaauuugaaaaucaucggcaucgacugccacaucgguucgcaacugaccgacuuaagcccacuggucgaagccugcgaacgcauuuugauuuugguugacgcucuugccgccgaaggcauuguuuuggaacauuuggacuuaggcggcggcgucggcauuguuuacaaagacgaaggcguccccgauuugggugcguaugcccgagcgguucaaaaacugauggggacacgccgucugaaacucauucuugagccaggccgcagcuuggucggcaacgcaggugcauugcugacgcgcgucgaauuugucaaacacggugaagagaaaaacuuugugauggucgaugcggcgaugaacgauuugaugcgcccagcccuauacgaugccuaccaccacaucgaagcgguugaaaccaaaaacauugagccucugaccgccaacaucgucggcccgauuugugaaaccggcgacuuccucggcaaagaccgcaccaucgccugcgaagaaggcgauuugcugcuuauccgcagcgcgggcgcauacggggccaguauggcuagcaauuacaacacgcgcaaccgugcggcggagguguugguugacggcggcggauacaaacucauccgccggcgcgaaaccuuggaacagcaaauggcaaacgaacucgccugccuauaa

SEQ ID NO: 130 - DNA, 324 bp, Neisseria gonorrhoeae
atgatgaccgaaagcgagtttatccgcgcgagcgaagcattatttgaacacatcgaagaccaaatcgacgaaaacggctgggatttcgactgccggtttgccggaaacgtcctgaccatcgaagccggagacggcacgcaaatcatcgtcaaccgccacacgcccaaccaagaattgtggattgccgcaaaaagcggcggctaccatttcgccgaacaaaacggcaaatggctggcaacgcgcgacagccgcgatttttacgacgttttaaacgaagccctgagcgcggcttcgggcgaagcggttgagattgccgaattgtga

SEQ ID NO: 131 - DNA, 324 bp, Neisseria gonorrhoeae
tcacaattcggcaatctcaaccgcttcgcccgaagccgcgctcagggcttcgtttaaaacgtcgtaaaaatcgcggctgtcgcgcgttgccagccatttgccgttttgttcggcgaaatggtagccgccgctttttgcggcaatccacaattcttggttgggcgtgtggcggttgacgatgatttgcgtgccgtctccggcttcgatggtcaggacgtttccggcaaaccggcagtcgaaatcccagccgttttcgtcgatttggtcttcgatgtgttcaaataatgcttcgctcgcgcggataaactcgctttcggtcatcat

SEQ ID NO: 132 - RNA, 324 nt, Neisseria gonorrhoeae
augaugaccgaaagcgaguuuauccgcgcgagcgaagcauuauuugaacacaucgaagaccaaaucgacgaaaacggcugggauuucgacugccgguuugccggaaacguccugaccaucgaagccggagacggcacgcaaaucaucgucaaccgccacacgcccaaccaagaauuguggauugccgcaaaaagcggcggcuaccauuucgccgaacaaaacggcaaauggcuggcaacgcgcgacagccgcgauuuuuacgacguuuuaaacgaagcccugagcgcggcuucgggcgaagcgguugagauugccgaauuguga

SEQ ID NO: 133 - DNA, 1566 bp, Neisseria gonorrhoeae
atgacccaagacaaaatcctcatcctcgacttcggttctcaagttacccggctgattgcccgccgcgtgcgcgaagcccacgtttactgcgaactgcattccttcgatatgcctttggacgaaatcaaagccttcaaccccaaaggcatcatcctttccggcggccctaattctgtttacgaatccgactatcaagccgataccggtatttttgatttgggcattccggttttgggcatctgctacggcatgcagtttatggcgcaccacttgggtggcgaagtgcagcccggcaaccagcgcgaattcggttacgcgcaagtcaaaaccatcgacagcggactgacacgcggcattcaagacgacgcgcccaacacactcgacgtatggatgagccacggcgacaaagtgtccaaactgcccgacggtttcgccgtcatcggcgataccccgtcctgcccgattgcaatgatggaaaacgccgaaaaacaattctacggcatccagttccaccccgaagttacccacaccaaacaaggccgcgccctgttgaaccgctttgtcttggatatttgcggcgcgcaaccgggctggacgatgcccaactacatcgaagaagccgttgccaaaatccgcgaacaagtcggcagcgacgaagtgattttaggtctgtccggcggcgtggactcttccgtagccgccgcgctgattcaccgcgccatcggcgaccaactgacctgcgtgttcgtcgatcacggtttgttgcgcctgaacgaaggcaaaatggtgatggatatgttcgcccgcaacttgggtgtgaaagtgatacacgtcgatgccgaagggcagtttatggcgaaactcgccggcgtgaccgaccctgagaaaaaacgcaaaatcatcggcgcggaatttatcgaagtatttgatgccgaagagaaaaaactcaccaacgccaaatggctggcgcaaggcacgatttaccccgacgtaatcgaatccgccggtgcgaaaaccaaaaaagcccacgccatcaaatcccaccacaacgtcggcggcctgcctgaaaatatgaagctcaaactgcttgagcccttgcgcgacttgttcaaagacgaagtgcgcgagttgggcgtggctttgggcctgccgcgcgaaatggtgtaccgccaccccttcccgggccccggtttgggtgtgcgcatcttgggcgaagtgaaaaaagaatacgccgacttgctgcgtcaggcggacgatattttcatccaagaattacgcaatactaccgacgaaaacggcacgtcttggtatgacctgaccagccaggcatttgccgtattcctgcccgtcaaatccgtcggcgtgatgggcgacggccgcacttacgactacgtcgtcgcactgcgcgcagtcatcaccagcgactttatgactgcacactgggcagagctgccatactcactgctcggccgcgtgtccaaccgcatcatcaacgaagtcaaaggcatcaaccgcgtggtgtacgatgtcagcggcaaaccgcccgccaccatcgagtgggaataa

SEQ ID NO: 134 - DNA, 1566 bp, Neisseria gonorrhoeae
ttattcccactcgatggtggcgggcggtttgccgctgacatcgtacaccacgcggttgatgcctttgacttcgttgatgatgcggttggacacgcggccgagcagtgagtatggcagctctgcccagtgtgcagtcataaagtcgctggtgatgactgcgcgcagtgcgacgacgtagtcgtaagtgcggccgtcgcccatcacgccgacggatttgacgggcaggaatacggcaaatgcctggctggtcaggtcataccaagacgtgccgttttcgtcggtagtattgcgtaattcttggatgaaaatatcgtccgcctgacgcagcaagtcggcgtattcttttttcacttcgcccaagatgcgcacacccaaaccggggcccgggaaggggtggcggtacaccatttcgcgcggcaggcccaaagccacgcccaactcgcgcacttcgtctttgaacaagtcgcgcaagggctcaagcagtttgagcttcatattttcaggcaggccgccgacgttgtggtgggatttgatggcgtgggcttttttggttttcgcaccggcggattcgattacgtcggggtaaatcgtgccttgcgccagccatttggcgttggtgagttttttctcttcggcatcaaatacttcgataaattccgcgccgatgattttgcgttttttctcagggtcggtcacgccggcgagtttcgccataaactgcccttcggcatcgacgtgtatcactttcacacccaagttgcgggcgaacatatccatcaccattttgccttcgttcaggcgcaacaaaccgtgatcgacgaacacgcaggtcagttggtcgccgatggcgcggtgaatcagcgcggcggctacggaagagtccacgccgccggacagacctaaaatcacttcgtcgctgccgacttgttcgcggattttggcaacggcttcttcgatgtagttgggcatcgtccagcccggttgcgcgccgcaaatatccaagacaaagcggttcaacagggcgcggccttgtttggtgtgggtaacttcggggtggaactggatgccgtagaattgtttttcggcgttttccatcattgcaatcgggcaggacggggtatcgccgatgacggcgaaaccgtcgggcagtttggacactttgtcgccgtggctcatccatacgtcgagtgtgttgggcgcgtcgtcttgaatgccgcgtgtcagtccgctgtcgatggttttgacttgcgcgtaaccgaattcgcgctggttgccgggctgcacttcgccacccaagtggtgcgccataaactgcatgccgtagcagatgcccaaaaccggaatgcccaaatcaaaaataccggtatcggcttgatagtcggattcgtaaacagaattagggccgccggaaaggatgatgcctttggggttgaaggctttgatttcgtccaaaggcatatcgaaggaatgcagttcgcagtaaacgtgggcttcgcgcacgcggcgggcaatcagccgggtaacttgagaaccgaagtcgaggatgaggattttgtcttgggtcat

SEQ ID NO: 135 - RNA, 1566 nt, Neisseria gonorrhoeae
augacccaagacaaaauccucauccucgacuucgguucucaaguuacccggcugauugcccgccgcgugcgcgaagcccacguuuacugcgaacugcauuccuucgauaugccuuuggacgaaaucaaagccuucaaccccaaaggcaucauccuuuccggcggcccuaauucuguuuacgaauccgacuaucaagccgauaccgguauuuuugauuugggcauuccgguuuugggcaucugcuacggcaugcaguuuauggcgcaccacuuggguggcgaagugcagcccggcaaccagcgcgaauucgguuacgcgcaagucaaaaccaucgacagcggacugacacgcggcauucaagacgacgcgcccaacacacucgacguauggaugagccacggcgacaaaguguccaaacugcccgacgguuucgccgucaucggcgauaccccguccugcccgauugcaaugauggaaaacgccgaaaaacaauucuacggcauccaguuccaccccgaaguuacccacaccaaacaaggccgcgcccuguugaaccgcuuugucuuggauauuugcggcgcgcaaccgggcuggacgaugcccaacuacaucgaagaagccguugccaaaauccgcgaacaagucggcagcgacgaagugauuuuaggucuguccggcggcguggacucuuccguagccgccgcgcugauucaccgcgccaucggcgaccaacugaccugcguguucgucgaucacgguuuguugcgccugaacgaaggcaaaauggugauggauauguucgcccgcaacuugggugugaaagugauacacgucgaugccgaagggcaguuuauggcgaaacucgccggcgugaccgacccugagaaaaaacgcaaaaucaucggcgcggaauuuaucgaaguauuugaugccgaagagaaaaaacucaccaacgccaaauggcuggcgcaaggcacgauuuaccccgacguaaucgaauccgccggugcgaaaaccaaaaaagcccacgccaucaaaucccaccacaacgucggcggccugccugaaaauaugaagcucaaacugcuugagcccuugcgcgacuuguucaaagacgaagugcgcgaguugggcguggcuuugggccugccgcgcgaaaugguguaccgccaccccuucccgggccccgguuugggugugcgcaucuugggcgaagugaaaaaagaauacgccgacuugcugcgucaggcggacgauauuuucauccaagaauuacgcaauacuaccgacgaaaacggcacgucuugguaugaccugaccagccaggcauuugccguauuccugcccgucaaauccgucggcgugaugggcgacggccgcacuuacgacuacgucgucgcacugcgcgcagucaucaccagcgacuuuaugacugcacacugggcagagcugccauacucacugcucggccgcguguccaaccgcaucaucaacgaagucaaaggcaucaaccgcgugguguacgaugucagcggcaaaccgcccgccaccaucgagugggaauaa

SEQ ID NO: 136 - DNA, 180 bp, Neisseria gonorrhoeae
atggccgttcaacaaaacaaaaaatccccttccaaacgcggtatgcaccgttcgcacgacgcactgaccgcgcccgcactgtttgtcgacagcacaaccggcgaagtacaccgcccgcaccacatctcccccaacggtatgtaccgcggccgcaaagtggtcaaagccaaaggcgaataa

SEQ ID NO: 137 - DNA, 180 bp, Neisseria gonorrhoeae
ttattcgcctttggctttgaccactttgcggccgcggtacataccgttgggggagatgtggtgcgggcggtgtacttcgccggttgtgctgtcgacaaacagtgcgggcgcggtcagtgcgtcgtgcgaacggtgcataccgcgtttggaaggggattttttgttttgttgaacggccat

SEQ ID NO: 138 - RNA, 180 nt, Neisseria gonorrhoeae
auggccguucaacaaaacaaaaaauccccuuccaaacgcgguaugcaccguucgcacgacgcacugaccgcgcccgcacuguuugucgacagcacaaccggcgaaguacaccgcccgcaccacaucucccccaacgguauguaccgcggccgcaaaguggucaaagccaaaggcgaauaa

SEQ ID NO: 139 - DNA, 501 bp, Neisseria gonorrhoeae
atgtcagaccctaatttgattgacccggaaatttttgccgccgaaaggcagaacctgcaaggcagttttctgctggaagaattggacgagcgagtcagtttgcacgattatcccgccgacaggcggaacaaaatatcgtttacactgaccggcggtcgcgaccggctgcaacgcctgttcctcgacctgaacgtcaaagccgatatgcccctgatttgccagagatgtatcaaacccatgccgttcatgctcgatgaaagcagccgtatcatcctgttttccgacgaagagtccttggacgaatccatgcttgccgacgaagaactcgaaggcatactgattgaaaaagaactcgacgtgcgcgcattggtagaagaccaaatcctgatgtccctgcccttttcgccgcgacacggacactgcggcaatacccttccggaatccgccaaccaagacaaacccaacccctttgctgttttggcgggtttgaaaagcagttaa

SEQ ID NO: 140 - DNA, 501 bp, Neisseria gonorrhoeae
ttaactgcttttcaaacccgccaaaacagcaaaggggttgggtttgtcttggttggcggattccggaagggtattgccgcagtgtccgtgtcgcggcgaaaagggcagggacatcaggatttggtcttctaccaatgcgcgcacgtcgagttctttttcaatcagtatgccttcgagttcttcgtcggcaagcatggattcgtccaaggactcttcgtcggaaaacaggatgatacggctgctttcatcgagcatgaacggcatgggtttgatacatctctggcaaatcaggggcatatcggctttgacgttcaggtcgaggaacaggcgttgcagccggtcgcgaccgccggtcagtgtaaacgatattttgttccgcctgtcggcgggataatcgtgcaaactgactcgctcgtccaattcttccagcagaaaactgccttgcaggttctgcctttcggcggcaaaaatttccgggtcaatcaaattagggtctgacat

SEQ ID NO: 141 - RNA, 501 nt, Neisseria gonorrhoeae
augucagacccuaauuugauugacccggaaauuuuugccgccgaaaggcagaaccugcaaggcaguuuucugcuggaagaauuggacgagcgagucaguuugcacgauuaucccgccgacaggcggaacaaaauaucguuuacacugaccggcggucgcgaccggcugcaacgccuguuccucgaccugaacgucaaagccgauaugccccugauuugccagagauguaucaaacccaugccguucaugcucgaugaaagcagccguaucauccuguuuuccgacgaagaguccuuggacgaauccaugcuugccgacgaagaacucgaaggcauacugauugaaaaagaacucgacgugcgcgcauugguagaagaccaaauccugaugucccugcccuuuucgccgcgacacggacacugcggcaauacccuuccggaauccgccaaccaagacaaacccaaccccuuugcuguuuuggcggguuugaaaagcaguuaa

SEQ ID NO: 142 - DNA, 86 bp, Neisseria gonorrhoeae
gccgacatggtgaaattggtagacacgctatcttgagggggtagtggccgtaggctgtgcgagttcaaatctcgctgtcggcacca

SEQ ID NO: 143 - DNA, 86 bp, Neisseria gonorrhoeae
tggtgccgacagcgagatttgaactcgcacagcctacggccactaccccctcaagatagcgtgtctaccaatttcaccatgtcggc

SEQ ID NO: 144 - RNA, 86 nt, Neisseria gonorrhoeae
gccgacauggugaaauugguagacacgcuaucuugaggggguaguggccguaggcugugcgaguucaaaucucgcugucggcacca

SEQ ID NO: 145 - DNA, 88 bp, Neisseria gonorrhoeae
ggaagcgtggcagagcggtttaatgcaacggtcttgaaaaccgtcgagggttgatagccctccgtgagttcgaatctcaccgcttccg

SEQ ID NO: 146 - DNA, 88 bp, Neisseria gonorrhoeae
cggaagcggtgagattcgaactcacggagggctatcaaccctcgacggttttcaagaccgttgcattaaaccgctctgccacgcttcc

SEQ ID NO: 147 - RNA, 88 nt, Neisseria gonorrhoeae
ggaagcguggcagagcgguuuaaugcaacggucuugaaaaccgucgaggguugauagcccuccgugaguucgaaucucaccgcuuccg

SEQ ID NO: 148 - DNA, 84 bp, Neisseria gonorrhoeae
gcccgggtggcggaattggtagacgcgccagcttcaggtgctggtatcctcacgggtatggaagttcgagtcttctcccgggca

SEQ ID NO: 149 - DNA, 84 bp, Neisseria gonorrhoeae
tgcccgggagaagactcgaacttccatacccgtgaggataccagcacctgaagctggcgcgtctaccaattccgccacccgggc

SEQ ID NO: 150 - RNA, 84 nt, Neisseria gonorrhoeae
gcccggguggcggaauugguagacgcgccagcuucaggugcugguauccucacggguauggaaguucgagucuucucccgggca

SEQ ID NO: 151 - DNA, 88 bp, Neisseria gonorrhoeae
agagaggtggatgagtggtttaagtcgcacgcctggaaagcgtgtatacgtgaatagcgtatcgagggttcgaatcccttcctctctg

SEQ ID NO: 152 - DNA, 88 bp, Neisseria gonorrhoeae
cagagaggaagggattcgaaccctcgatacgctattcacgtatacacgctttccaggcgtgcgacttaaaccactcatccacctctct

SEQ ID NO: 153 - RNA, 88 nt, Neisseria gonorrhoeae
agagagguggaugagugguuuaagucgcacgccuggaaagcguguauacgugaauagcguaucgaggguucgaaucccuuccucucug

SEQ ID NO: 154 - DNA, 996 bp, Neisseria meningitides
atgaaaaaatccctgattgccctgactttggcagcccttcctgttgcagcaatggctgacgttaccctgtacggcaccatcaaagccggcgtagaaacttcccgctctgtatttcaccagaacggccaagttactgaagttacaaccgctaccggcatcgttgatttgggttcgaaaatcggcttcaaaggccaagaagacctcggtaacggcctgaaagccatttggcaggttgagcaaaaagcatctatcgccggtactgactccggttggggcaaccgccaatccttcatcggcttgaaaggcggcttcggtaaattgcgcgtcggtcgtttgaacagcgtcctgaaagacaccggcgacatcaatccttgggatagcaaaagcgactatttgggtgtaaacaaaattgccgaacccgaggcacgcctcatttccgtacgctacgattctcccgaatttgccggcctcagcggcagcgtacaatacgcgcttaacgacaatgcaggcagacataacagcgaatcttaccacgccggcttcaactacaaaaacggtggcttcttcgtgcaatatggcggtgcctataaaagacatcatcaagtgcaagagggcttgaatattgagaaataccagattcaccgtttggtcagcggttacgacaatgatgccctgtacgcttccgtagccgtacagcaacaagacgcgaaactgactgatgcttccaattcgcacaactctcaaaccgaagttgccgctaccttggcataccgcttcggcaacgtaacgccccgagtttcttacgcccacggcttcaaaggtttggttgatgatgcagacataggcaacgaatacgaccaagtggttgtcggtgcggaatacgacttctccaaacgcacttctgccttggtttctgccggttggttgcaagaaggcaaaggcgaaaacaaattcgtagcgactgccggcggtgtcggtctgcgccacaaattctaa

SEQ ID NO: 155 - DNA, 996 bp, Neisseria meningitides; identical to SEQ ID NO: 154

SEQ ID NO: 156 - RNA, 996 nt, Neisseria meningitides
uuagaauuuguggcgcagaccgacaccgccggcagucgcuacgaauuuguuuucgccuuugccuucuugcaaccaaccggcagaaaccaaggcagaagugcguuuggagaagucguauuccgcaccgacaaccacuuggucguauucguugccuaugucugcaucaucaaccaaaccuuugaagccgugggcguaagaaacucggggcguuacguugccgaagcgguaugccaagguagcggcaacuucgguuugagaguugugcgaauuggaagcaucagucaguuucgcgucuuguugcuguacggcuacggaagcguacagggcaucauugucguaaccgcugaccaaacggugaaucugguauuucucaauauucaagcccucuugcacuugaugaugucuuuuauaggcaccgccauauugcacgaagaagccaccguuuuuguaguugaagccggcgugguaagauucgcuguuaugucugccugcauugucguuaagcgcguauuguacgcugccgcugaggccggcaaauucgggagaaucguagcguacggaaaugaggcgugccucggguucggcaauuuuguuuacacccaaauagucgcuuuugcuaucccaaggauugaugucgccggugucuuucaggacgcuguucaaacgaccgacgcgcaauuuaccgaagccgccuuucaagccgaugaaggauuggcgguugccccaaccggagucaguaccggcgauagaugcuuuuugcucaaccugccaaauggcuuucaggccguuaccgaggucuucuuggccuuugaagccgauuuucgaacccaaaucaacgaugccgguagcgguuguaacuucaguaacuuggccguucuggugaaauacagagcgggaaguuucuacgccggcuuugauggugccguacaggguaacgucagccauugcugcaacaggaagggcugccaaagucagggcaaucagggauuuuuucau

SEQ ID NO: 157 - DNA, 234 bp, Neisseria meningitides
ttaagcttcgccgcgagcacgcaaatcagccaatacgacatcaatgcctactttgtcgatggtacgcagtgcagcgttggaaacgcgcaggcgaacccagcggttttcactttctacccaaaaacgacgtgattgcaagttgggcaaaaaacggcgtttggttttgttgttggcgtgcgatacgttgttgccggacatcgggcgtttgccggtcactttgcaaactcgtgccat

SEQ ID NO: 158 - DNA, 234 bp, Neisseria meningitides
atggcacgagtttgcaaagtgaccggcaaacgcccgatgtccggcaacaacgtatcgcacgccaacaacaaaaccaaacgccgttttttgcccaacttgcaatcacgtcgtttttgggtagaaagtgaaaaccgctgggttcgcctgcgcgtttccaacgctgcactgcgtaccatcgacaaagtaggcattgatgtcgtattggctgatttgcgtgctcgcggcgaagcttaa

SEQ ID NO: 159 - RNA, 234 nt, Neisseria meningitides
uuaagcuucgccgcgagcacgcaaaucagccaauacgacaucaaugccuacuuugucgaugguacgcagugcagcguuggaaacgcgcaggcgaacccagcgguuuucacuuucuacccaaaaacgacgugauugcaaguugggcaaaaaacggcguuugguuuuguuguuggcgugcgauacguuguugccggacaucgggcguuugccggucacuuugcaaacucgugccau

SEQ ID NO: 160 - DNA, 23 bp, Artificial Sequence
  Synthetic Polynucleotide
gctacgattctcccgaatttgcc

SEQ ID NO: 161 - DNA, 20 bp, Artificial Sequence
  Synthetic Polynucleotide
ccgcckaccaaacggtgaac

SEQ ID NO: 162 - DNA, 20 bp, Artificial Sequence
  Synthetic Polynucleotide
ttgcccaacttgcaatcacg

SEQ ID NO: 163 - DNA, 21 bp, Artificial Sequence
  Synthetic Polynucleotide
agcacgcaaatcagccaatac

SEQ ID NO: 164 - DNA, 19 bp, Artificial Sequence
  Synthetic Polynucleotide
gctttggaaaaagcagccg

SEQ ID NO: 165 - DNA, 20 bp, Artificial Sequence
  Synthetic Polynucleotide
ggttttgttgtcggtcaggc

SEQ ID NO: 166 - DNA, 19 bp, Artificial Sequence
  Synthetic Polynucleotide
gacttttgccgctgctttg

SEQ ID NO: 167 - DNA, 19 bp, Artificial Sequence
  Synthetic Polynucleotide
gcgcattattcgtgtgcag

SEQ ID NO: 168 - DNA, 19 bp, Artificial Sequence
  Synthetic Polynucleotide
aaagccttgggtattgcgg

SEQ ID NO: 169 - DNA, 19 bp, Artificial Sequence
  Synthetic Polynucleotide
tgaccaaagcaaccggaac

SEQ ID NO: 170 - DNA, 20 bp, Artificial Sequence
  Synthetic Polynucleotide
gaggcttcccccgtattgag

SEQ ID NO: 171 - DNA, 20 bp, Artificial Sequence
  Synthetic Polynucleotide
ttcaaaagccgcttcgttcg

SEQ ID NO: 172 - DNA, 20 bp, Artificial Sequence
  Synthetic Polynucleotide
actgcgttctgaactgggtg

SEQ ID NO: 173 - DNA, 18 bp, Artificial Sequence
  Synthetic Polynucleotide
ggcggtcaatttcacgcg

SEQ ID NO: 174 - DNA, 20 bp, Artificial Sequence
  Synthetic Polynucleotide
gcatctaagcgcgaaactcg

SEQ ID NO: 175 - DNA, 20 bp, Artificial Sequence
  Synthetic Polynucleotide
ccccacctatcaacgtcctg

SEQ ID NO: 176 - DNA, 19 bp, Artificial Sequence
  Synthetic Polynucleotide
gtttcagcggcagcattca

SEQ ID NO: 177 - DNA, 22 bp, Artificial Sequence
  Synthetic Polynucleotide
ccggaactggtttcatctgatt

SEQ ID NO: 178 - DNA, 1047 bp, Neisseria gonorrhoeae
atgaaaaaatccctgattgccctgactttggcagcccttcctgttgcggcaacggccgatgttaccctgtacggcgccatcaaagccggcgtacaaacttaccgttctgtagaacatcgggaaggcaaagtaattggcgtgggaaccggcagcgaaatctccgacttcggttcaaaaatcggcttcaaaggccaagaagacctcggcaacggcctgaaagccatttggcagttggaacaaggcgcctccgtcgccggcactaacagcggctggggcaacaaacaatccttcatcggcttgaagggcggcttcggcaccatccgcgccggtagcctgaacagccccctgaaaaacaccaagaacaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcagggtcaaacggcgaatcttaccacgttggcttgaactaccgaaacaacggcttcttcgcacaatacgccggcttgttccaaagatacggcgaaggcactaaaaaaatggaatacgatggtcaaacttataatatccccagtttgtttgttgaaaaactgcaagttcaccgtttggtcggcggttacgacaataatgccctgtacgcctccgtagccgcacaacaacaagatgccaaattgtatggagcaatgagcggtaattcgcacaactctcaaaccgaagttgccgctaccgtagcataccgtttcggcaacgtaacgccccgtgtttcttacgcccacggcttcaaaggcactgttgatgatgcaaaccacgacaatacttacgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 179 - DNA, 1047 bp, Neisseria gonorrhoeae
atgaaaaaatccctgattgccctgactttggcagcccttcctgttgcggcaacggccgatgttaccctgtacggcgccatcaaagccggcgtacaaacttaccgttctgtagaacatcgggaaggcaaagtaattggcgtgggaaccggcagcgaaatctccgacttcggttcaaaaatcggcttcaaaggccaagaagacctcggcaacggcctgaaagccatttggcagttggaacaaggcgcctccgtcgccggcactaacagcggctggggcaacaaacaatccttcatcggcttgaagggcggcttcggcaccatccgcgccggtagcctgaacagccccctgaaaaacaccaagaacaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcagggtcaaacggcgaatcttaccacgttggcttgaactaccgaaacaacggcttcttcgcacaatacgccggcttgttccaaagatacggcgaaggcactaaaaaaatggaatacgataatcaattttataatatccccagtttgtttgttgaaaaactgcaagttcaccgtttggtcggcggttacgacaataatgccctgtacgcctccgtagccgcacaacaacaagatgccaaattgtatggagcaatgagcggtaattcgcacaactctcaaaccgaagttgccgctaccgtagcataccgtttcggcaacgtaacgccccgtgtttcttacgcccacggcttcaaaggcactgttgatgatgcaaaccacgacaatacttacgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 180 - DNA, 1047 bp, Neisseria gonorrhoeae
atgaaaaaatccctgattgccctgactttggcagcccttcctgttgcggcaacggccgatgttaccctgtacggcgccatcaaagccggcgtacaaacttaccgttctgtagaacatcgggaaggcaaagtagttggcgtgggaaccggcagcgaaatctccgacttcggttcaaaaatcggcttcaaaggtcaagaagacctcggcaacggcctgaaggccgtttggcagttggaacaaggcgcctccgtcgccggcgctaacaccggctggggcaacaaacaatccttcgtcggcttgaaaggcggcttcggcaccatccgcgtcggcagcctgaacagccccctgaaaaacaccggtgccaacgtcaatgcttgggaatccggcaaatataccggcgagcttctggaaatcagcaaaatggccggacgggaacaccgctacctgtccgcacgctacgattctcccgaatttgccggcttcagcggcagcgtacaatacgcacctaaaggtaattcaggctcaaacggcgaatcttaccacgttggcttgaactaccgaaacagcggcttcttcgcacaatacgccggcttgttccaaagatacggcgaaggcactaaaaaaatcgaatacgatgatcaaacttatagtatgcccagtctgtttgttgaaaaactgcaagttcaccgtttggtaggcggttacgacaataatgccctgtacgtttccgtagccgcacaacaacaagatgccaaattgtatggagcaacgagggttaattcgcacaactctcaaaccgaagttgccgctaccgcggcataccgtttcggcaatgtaacgccccgcgtttcttacgcccacggcttcaaaggcactgttgatgatgcaaaccacgacaatacttatgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 181 - DNA, 1047 bp, Neisseria gonorrhoeae
atgaaaaaatccctgattgccctgactttggcagcccttcctgttgcggcaacggccgatgtcaccctgtacggcgccatcaaagccggcgtacaaacttaccgttctgtagaacatcgggaaggcaaagtagttggcgtgggaaccggcagcgaaatctccgacttcggttcaaaaatcggcttcaaaggccaagaagacctcggcaacggcctgaaagccatttggcagttggaacaaggcgcctccgtcgccggcactaacagcggctggggcaacaaacaatccttcatcggcttgaagggcggcttcggcaccatccgcgccggtagcctgaacagccccctgaaaaacaccaagaacaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcagggtcaaacggcgaatcttaccacgttggcttgaactaccgaaacaacggcttcttcgcacaatacgccggcttgttccaaagatacggcgaaggcactaaaaaaatggaatacgatggtcaaacttataatatccccagtttgtttgttgaaaaactgcaagttcaccgtttggtcggcggttacgacaataatgccctgtacgcctccgtagccgcacaacaacaagatgccaaattgtatggagcaatgagcggtaattcgcacaactctcaaaccgaagttgccgctaccgtagcataccgtttcggcaacgtaacgccccgtgtttcttacgcccacggcttcaaaggcactgttgatgatgcaaaccacgacaatacttacgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 182 - DNA, 1047 bp, Neisseria gonorrhoeae
atgaaaaaatccctgattgccctgactttggcagcccttcctgttgcggcaacggccgatgtcaccctgtacggcgccatcaaagccggcgtacaaacttaccgttctgtagaacatcgggaaggcaaagtaattggcgtgggaaccggcagcgaaatctccgacttcggttcaaaaatcggcttcaaaggccaagaagacctcggcaacggcctgaaagccatttggcagttggaacaaggcgcctccgtcgccggcactaacagcggctggggcaacaaacaatccttcatcggcttgaagggcggcttcggcaccatccgcgccggtagcctgaacagccccctgaaaaacaccaagaacaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcagggtcaaacggcgaatcttaccacgttggcttgaactaccgaaacaacggcttcttcgcacaatacgccggcttgttccaaagatacggcgaaggcactaaaaaaatggaatacgatggtcaaacttataatatccccagtttgtttgttgaaaaactgcaagttcaccgtttggtcggcggttacgacaataatgccctgtacgcctccgtagccgcacaacaacaagatgccaaattgtatggagcaatgaacggtaattcgcacaactctcaaaccgaagttgccgctaccgtagcataccgtttcggcaacgtaacgccccgtgtttcttacgcccacggcttcaaaggcactgttgatgatgcaaaccacgacaatacttacgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 183 - DNA, 1047 bp, Neisseria gonorrhoeae
atgaaaaaatccctgattgccctgactttggcagcccttcctgttgcggcaacggccgatgtcaccctgtacggcgccatcaaagccggcgtacaaacttaccgttctgtagaacatcgggaaggcaaagtaattggcgtgggaaccggcagcgaaatctccgacttcggttcaaaaatcggcttcaaaggccaagaagacctcggcaacggcctgaaagccatttggcagttggaacaaggcgcctccgtcgccggcactaacagcggctggggcaacaaacaatccttcatcggcttgaagggcggcttcggcaccatccgcgccggtagcctgaacagccccctgaaaaacaccaagaacaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcagggtcaaacggcgaatcttaccacgttggcttgaactaccgaaacaacggcttcttcgcacaatacgccggcttgttccaaagatacggcgaaggcactaaaaaaatggaatacgatggtcaaacttataatatccccagtttgtttgttgaaaaactgcaagttcaccgtttggtcggcggttacgacaataatgccctgtacgcctccgtagccgcacaacaacaagatgccaaattgtatggagcaatgagcggtaattcgcacaactctcaaaccgaagttgccgctaccgtagcataccgtttcggcaacgtaacgccccgtgtttcttacgcccacggcttcaaaggcactgttgatgatgcaaaccacgacaatacttacgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 184 - DNA, 1047 bp, Neisseria gonorrhoeae; identical to SEQ ID NO: 183

SEQ ID NO: 185 - DNA, 1047 bp, Neisseria gonorrhoeae; identical to SEQ ID NO: 181

SEQ ID NO: 186 - DNA, 1047 bp, Neisseria gonorrhoeae; identical to SEQ ID NO: 182

SEQ ID NO: 187 - DNA, 1047 bp, Neisseria gonorrhoeae; identical to SEQ ID NO: 183

SEQ ID NO: 188 - DNA, 1047 bp, Neisseria gonorrhoeae; identical to SEQ ID NO: 180

SEQ ID NO: 189 - DNA, 1047 bp, Neisseria gonorrhoeae; identical to SEQ ID NO: 181

SEQ ID NO: 190 - DNA, 1047 bp, Neisseria gonorrhoeae
atgaaaaaatccctgattgccctgactttggcagcccttcctgttgcggcaacggccgatgtcaccctgtacggcgccatcaaagccggcgtacaaacttaccgttctgtagaacatcgggaaggcaaagtagttggcgtgggaaccggcagcgaaatctccgacttcggttcaaaaatcggcttcaaaggccaagaagacctcggcaacggcctgaaggccgtttggcagttggaacaaggcgcctccgtcgccggcactaacaccggctggggcaacaaacaatccttcgtcggcttgaagggcggcttcggcaccatccgcgccggtagcctgaacagccccctgaaaaacaccaagaacaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcagggtcaaacggcgaatcttaccacgttggcttgaactaccgaaacaacggcttcttcgcacaatacgccggcttgttccaaagatacggcgaaggcactaaaaaaatcgaatacgatggtcaaacttataatatccccagtttgtttgttgaaaaactgcaagttcaccgtttggtcggcggttacgacaataatgccctgtacgcctccgtagccgcacaacaacaagatgccaaattgtatggagcaatgagcggtaattcgcacaactctcaaaccgaagttgccgctaccgtagcataccgtttcggcaacgtaacgccccgtgtttcttacgcccacggcttcaaaggcactgttgatgatgcaaaccacgacaatacttacgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 191 - DNA, 1047 bp, Neisseria gonorrhoeae; identical to SEQ ID NO: 183

SEQ ID NO: 192 - DNA, 1047 bp, Neisseria gonorrhoeae
atgaaaaaatccctgattgccctgactttggcagcccttcctgttgcggcaacggccgatgttaccctgtacggcgccatcaaagccggcgtacaaacttaccgttctgtagaacatcgggaaggcaaagtagttggcgtggaaaccggcagcgaaatctccgacttcggttcaaaaatcggcttcaaaggtcaagaagacctcggcaacggcctgaaggccgtttggcagttggaacaaggtgcctccgtcgccggcactaacaccggctggggcaacaaacaatccttcgtcggcttgaaaggcggcttcggcaccatccgcgtcggcagcctgaacagccccctgaaaaacaccggtgccaacgtcaatgcttgggaatccggcaaatataccggcgagtttctggaaatcagcaaaatggccagacgggaacaccgctacctgtccgcacgctacgattctcccgaatttgccggcttcagcggcagcgtacaatacgcacctaaagataattcaggctcaaacggcgaatcttaccacgttggcttgaactaccgaaacggcggcttcttcgcacaatacgccggcttgttccaaagatacggcgaaggcactaaaaaaatcgaatacgctggtcaatattatagtatccccagcctgtttgttgaaaaactgcaagttcaccgtttggtcggcggttacgacaataatgccctgtacgcctccgtagccgcacaacaacaagatgccaaattgtatggaacatggagtgctaattcgcacaactctcaaaccgaagttgccgctaccgcggcataccgtttcggcaacttaacgccccgcgtttcttacgcccacggcttcaaaggctctgttcatagtgcagactacgacaatacttatgaccaagtggttgtcggtgcggaatacgacttctccaaacgcacttctgccttggtttctgccggctggttgcaagaaggcaaaggcgcagaaaaagtcgtatcgactgccagcgccgtcgttctgcgccacaaattctaa

SEQ ID NO: 193 - DNA, 1047 bp, Neisseria gonorrhoeae; identical to SEQ ID NO: 181

SEQ ID NO: 194 - DNA, 1047 bp, Neisseria gonorrhoeae
atgaaaaaatccctgattgccctgactttggcagcccttcctgttgcggcaacggccgatgtcaccctgtacggcgccatcaaagccggcgtacaaacttaccgttctgtagaacatcgggaaggcaaagtagttgacgtgggaaccggcagcgaaatctccgacttcggttcaaaaatcggcttcaaaggccaagaagacctcggcaacggcctgaaggccgtttggcagttggaacaaggcgcctccgtcgccggcgctaacagcggctggggcaacaaacaatccttcgtcggcttgaagggcggcttcggcaccatccgcgccggtagcctgaacagccccctgaaaaacaccggcagcaaagtcaatgcttgggaatccggcaaatttaccggcagtgtgctgaaaatcagcggaatggccgaacgggaacaccgctacctgtccgcacgctacgattctcccgaatttgccggcttcagcggcagcgtacaatacgcacctaaagacaattcaggctcaaacggcgaatcttaccacgttggcttgaactaccaaaacagcggcttcttcgcacaatacgccggcttgttccaaagatacggcgaaggcactaaaaaaatcgaatacgatggtcaaacttatagtatgcccagtctgtttgttgaaaaactgcaagttcaccgtttggtaggcggttacgacaataatgccctgtacgcctccgtagccgcacaacaacaagatgccaaattgtatggaacatggagtgctaattcgcacaactctcaaaccgaagttgccgctaccgtggcataccgtttcggcaacgtaacgccccgtgtttcttacgcccacggcttcaaaggcactgttgatgatgcaaaccacgacaatacttacgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 195 - DNA, 1047 bp, Neisseria gonorrhoeae
atgaaaaaatccctgattgccctgactttggcagcccttcctgttgcggcaacggccgatgtcaccctgtacggcgccatcaaagccggcgtacaaacttaccgttctgtagaacatcgggaaggcaaagtaattggcgtggaaaccggcagcgaaatctccgacttcggttcaaaaatcggcttcaaaggccaagaagacctcggcaacggcctgaaagccatttggcagttggaacaaggcgcctccgtcgccggcactaacagcggctggggcaacaaacaatccttcatcggcttgaagggcggcttcggcaccatccgcgccggtagcctgaacagccccctgaaaaacaccaagaacaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcagggtcaaacggcgaatcttaccacgttggcttgaactaccgaaacaacggcttcttcgcacaatacgccggcttgttccaaagatacggcgaaggcactaaaaaaatggaatacgatggtcaatcttataatatccccagtttgtttgttgaaaaactgcaagttcaccgtttggtcggcggttacgacaataatgccctgtacgcctccgtagccgcacaacaacaagatgccaaattgtatggagcaatgagcggtaattcgcacaactctcaaaccgaagttgccgctaccgtagcataccgtttcggcaacgtaacgccccgtgtttcttacgcccacggcttcaaaggcactgttgatgatgcaaaccacgacaatacttacgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 196 - DNA, 1047 bp, Neisseria gonorrhoeae; identical to SEQ ID NO: 183

SEQ ID NO: 197 - DNA, 1047 bp, Neisseria gonorrhoeae; identical to SEQ ID NO: 181

SEQ ID NO: 198 - DNA, 1047 bp, Neisseria gonorrhoeae
atgaaaaaatccctgattgccctgactttggcagcccttcctgttgcggcaacggccgatgtcaccctgtacggcgccatcaaagccggcgtacaaacttaccgttctgtagaacatcgggaaggcaaagtacttggcgtgggaaccggcagcgaaatctccgacttcggttcaaaaatcggcttcaaaggccaagaagacctcggcaacggcctgaaagccatttggcagttggaacaaggcgcctccgtcgccggcactaacagcggctggggcaacaaacaatccttcatcggcttgaagggcggcttcggcaccatccgcgccggtagcctgaacagccccctgaaaaacaccaagaacaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcagggtcaaacggcgaatcttaccacgttggcttgaactaccgaaacaacggcttcttcgcacaatacgccggcttgttccaaagatacggcgaaggcactaaaaaaatggaatacgatggtcaaacttataatatccccagtttgtttgttgaaaaactgcaagttcaccgtttggtcggcggttacgacaataatgccctgtacgcctccgtagccgcacaacaacaagatgccaaattgtatggagcaatgagcggtaattcgcacaactctcaaaccgaagttgccgctaccgtagcataccgtttcggcaacgtaacgccccgtgtttcttacgcccacggcttcaaaggcactgttgatgatgcaaaccacgacaatacttacgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 199 - DNA, 1047 bp, Neisseria gonorrhoeae; identical to SEQ ID NO: 183

SEQ ID NO: 200 - DNA, 1047 bp, Neisseria gonorrhoeae
atgaaaaaatccctgattgccctgactttggcagcccttcctgttgcggcaacggccgatgttaccctgtacggcgccatcaaagccggcgtacaaacttaccgttctgtagaacatcgggaaggcaaagtagttggcgtgggaaccggcagcgaaatctccgacttcggttcaaaaatcggcttcaaaggtcaagaagacctcggcaacggcctgaaggccgtttggcagttggaacaaggcgcctccgtcgccggcactaacaccggctggggcaacaaacaatccttcgtcggcttgaaaggcggcttcggcaccatccgcgtcggcagcctgaacagccccctgaaaaacaccggtgccaacgtcaatgcttgggaatccggcaaatataccggcgagcttctggaaatcagcaaaatggccggacgggaacaccgctacctgtccgcacgctacgattctcccgaatttgccggcttcagcggcagcgtacaatacgcacctaaaggtaattcaggctcaaacggcgaatcttaccacgttggcttgaactaccgaaacaacggcttcttcgcacaatacgccggcttgttccaaagatacggcgaaggcactaaaaaaatggaatacgatggtcaaacttataatatccccagtttgtttgttgaaaaactgcaagttcaccgtttggtcggcggttacgacaataatgccctgtacgcctccgtagccgcacaacaacaagatgccaaattgtatggagcaatgagcggtaattcgcacaactctcaaaccgaagttgccgctaccgtagcataccgtttcggcaacgtaacgccccgtgtttcttacgcccacggcttcaaaggcactgttgatgatgcaaaccacgacaatacttacgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 201 - DNA, 1047 bp, Neisseria gonorrhoeae; identical to SEQ ID NO: 181

SEQ ID NO: 202 - DNA, 1047 bp, Neisseria gonorrhoeae; identical to SEQ ID NO: 183

SEQ ID NO: 203 - DNA, 1047 bp, Neisseria gonorrhoeae
atgaaaaaatccctgattgccctgactttggcagcccttcctgttgcggcaacggccgatgtcaccctgtacggcaccatcaaagccggcgtacaaacttaccgttctgtagaacatacaaaaggcaaggtaagtaaagtggaaaccggcagcgaaatcgccgacttcggttcaaaaatcggcttcaaaggccaagaagacctcggcaacggcctgaaggccgtttggcagttggaacaaggtgcctccgtcgccggcactaacaccggctggggcaacaaacaatccttcgtcggcttgaagggcggcttcggtaccatccgcgccggtagcctgaacagccccctgaaaaacaccaaggacaacgtcaatgcttgggaatccggcaaatttaccggcgatgtgctggaaatcagcggaatggccaaacgggaacaccgctacctgtccgtacgctacgattctcccgaatttgccggcttcagcggcagcgtacaatacgcacctaaagataattcaggctcaaacggcgaatcttaccacgttggcttgaactaccgaaacggcggcttcttcgcacaatacgccggcttgttccaaagatacggcgaaggcactaaaaaaatcgaatacgataatcaattttatagtgtccccagcctgtctgttgaaaaactgcaagttcaccgtttggtcggcggttacgacaataatgccctgtacgcctccgtagccgcacaacaacaagatgccaaattgtatggaacatggcgtgctaattcgcacaactctcaaaccgaagttgctgctaccgcggcataccgtttcggcaacttaacgccccgcgtttcttacgcccacggcttcaaaggctctgttcatagtgcagactacgacaatacttatgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 204 - DNA, 1047 bp, Neisseria gonorrhoeae; identical to SEQ ID NO: 183

SEQ ID NO: 205 - DNA, 1047 bp, Neisseria gonorrhoeae; identical to SEQ ID NO: 181

SEQ ID NO: 206 - DNA, 1047 bp, Neisseria gonorrhoeae
atgaaaaaatccctgattgccctgactttggcagcccttcctgttgcggcaacggccgatgtcaccctgtacggcaccatcaaagccggcgtacaaacttaccgttctgtagaacatacaaaaggcaaggtaagtaaagtggaaaccggcagcgaaatcgccgacttcggttcaaaaatcggcttcaaaggccaagaagacctcggcaacggcctgaaggccgtttggcagttggaacaaggtgcctccgtcgccggcactaacaccggctggggcaacaaacaatccttcgtcggcttgaagggcggcttcggtaccatccgcgccggtagcctgaacagccccctgaaaaacaccaaggacaacgtcaatgcttgggaatccggcaaatttaccggcgatgtgctggaaatcagcggaatggccaaacgggaacaccgctacctgtccgtacgctacgattctcccgaatttgccggcttcagcggcagcgtacaatacgcacctaaagataattcaggctcaaacggcgaatcttaccacgttggcttgaactaccgaaacggcggcttcttcgcacaatacgccggcttgttccaaagatacggcgaaggcactaaaaaaatcgaatacaataatcaattttatagtgtccccagcctgtctgttgaaaaactgcaagttcaccgtttggtcggcggttacgacaataatgccctgtacgcctccgtagccgcacaacaacaagatgccaaattgtatggaacatggcgtgctaattcgcacaactctcaaaccgaagttgctgctaccgcggcataccgtttcggcaacttaacgccccgcgtttcttacgcccacggcttcaaaggctctgttcatagtgcagactacgacaatacttatgaccaagtggttgtcggtgcggaatacgacttctccaaacgcacttctgccttggtttctgccggctggttgcaagcaggcaaaggcgcagacaaaatcgtatcgactgccagcgccgtcgttctgcgccacaaattctaa

SEQ ID NO: 207 - DNA, 1047 bp, Neisseria gonorrhoeae
atgaaaaaatccctgattgccctgactttggcagcccttcctgttgcggcaacggccgatgttaccctgtacggcgccatcaaagccggcgtacaaacttaccgttctgtagaacatcgggaaggcaaagtagttggcgtggaaaccggcagcgaaatctccgacttcggttcaaaaatcggcttcaaaggtcaagaagacctcggcaacggcctgaaggccgtttggcagttggaacaaggtgcctccgtcgccggcactaacaccggctggggcaacaaacaatccttcgtcggcttgaagggcggcttcggtaccatccgcgccggtagcctgaacagccccctgaaaaacaccaacgccaacgtcaatgcttgggaatccggcaaatttaccggcaatgtgctggaaatcagcaaaatggccgaacgggaacaccgctacctgtccgtacgctacgattctcccgaatttgccggcttcagcggcagcgtacaatacgcacctaaagataattcaggctcaaacggcgaatcttaccacgttggcttgaactaccgaaacggcggcttcttcgcacaatacgccggcttgttccaaagatacggcgaaggcactaaaaaaatcgaatacgataatcaatcttatagtatccccagcctgtttgttgaaaaactgcaagttcaccgtttggtaggcggttacgacaataatgccctgtacgtctccgtagccgcacaacaacaagatgccaaattgtatggatcaatgagcggtaattcgcacaactctcaaaccgaagttgccgctaccgcggcataccgtttcggcaacgtaacgccccgcgtttcttacgcccacggcttcaaaggcagtgttgatagtgcaaaccacgacaatacttatgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 208 - DNA, 1047 bp, Neisseria gonorrhoeae
atgaaaaaatccctgattgccctgactttggcagcccttcctgttgcggcaacggccgatgttaccctgtacggcgccatcaaagccggcgtacaaacttaccgttctgtagaacatcgggaaggcaaagtaattggcgtgggaaccggcagcgaaatctccgacttcggttcaaaaatcggcttcaaaggccaagaagacctcggcaacggcctgaaagccatttggcagttggaacaaggcgcctccgtcgccggcactaacagcggctggggcaacaaacaatccttcatcggcttgaagggcggcttcggcaccatccgcgccggtagcctgaacagccccctgaaaaacaccaagaacaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcagggtcaaacggcgaatcttaccacgttggcttgaactaccgaaacaacggcttcttcgcacaatacgccggcttgttccaaagatacggcgaaggcactaaaaaaatggaatacgataatcaaacttataatatccccagtttgtttgttgaaaaactgcaagttcaccgtttggtcggcggttacgacaataatgccctgtacgcctccgtagccgcacaacaacaagatgccaaattgtatggagcaatgagcggtaattcgcacaactctcaaaccgaagttgccgctaccgtagcataccgtttcggcaacgtaacgccccgtgtttcttacgcccacggcttcaaaggcactgttgatgatgcaaaccacgacaatacttacgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 209 - DNA, 1047 bp, Neisseria gonorrhoeae
atgaaaaaatccctgattgccctgactttggcagcccttcctgttgcggcaacggccgatgttaccctgtacggcgccatcaaagccggcgtacaaacttaccgttctgtagaacatcgggaaggcaaagtagttggcgtgggaaccgacagcgaaatctccgacttcggttcaaaaatcggcttcaaaggccaagaagacctcggcaacggcctgaaagccatttggcagttggaacaaggcgcctccgtcgccggcactaacagcggctggggcaacaaacaatccttcatcggcttgaagggcggcttcggcaccatccgcgccggtagcctgaacagccccctgaaaaacaccaagaacaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcagggtcaaacggcgaatcttaccacgttggcttgaactaccgaaacaacggcttcttcgcacaatacgccggcttgttccaaagatacggcgaaggcactaaaaaaatggaatacgataatcaattttataatatccccagtttgtttgttgaaaaactgcaagttcaccgtttggtcggcggttacgacaataatgccctgtacgcctccgtagccgcacaacaacaagatgccaaattgtatggagcaatgagcggtaattcgcacaactctcaaaccgaagttgccgctaccgtagcataccgtttcggcaacgtaacgccccgtgtttcttacgcccacggcttcaaaggcactgttgatgatgcaaaccacgacaatacttacgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 210 - DNA, 1047 bp, Neisseria gonorrhoeae
atgaaaaaatccctgattgccctgactttggcagcccttcctgttgcggcaacggccgatgtcaccctgtacggcgccatcaaagccggcgtacaaacttaccgttctgtagaacatcgggaaggcaaagtagttggcgtgggaaccgacagcgaaatctccgacttcggttcaaaaatcggcttcaaaggccaagaagacctcggcaacggcctgaaagccatttggcagttggaacaaggcgcctccgtcgccggcactaacagcggctggggcaacaaacaatccttcatcggcttgaagggcggcttcggcaccatccgcgccggtagcctgaacagccccctgaaaaacaccaagaacaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcagggtcaaacggcgaatcttaccacgttggcttgaactaccgaaacaacggcttcttcgcacaatacgccggcttgttccaaagatacggcgaaggcactaaaaaaatggaatacaataatcaattttataatatccccagtttgtttgttgaaaaactgcaagttcaccgtttggtcggcggttacgacaataatgccctgtacgcctccgtagccgcacaacaacaagatgccaaattgtatggagcaatgagcggtaattcgcacaactctcaaaccgaagttgccgctaccgtagcataccgtttcggcaacgtaacgccccgtgtttcttacgcccacggcttcaaaggcactgttgatgatgcaaaccacgacaatacttacgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 211 - DNA, 1047 bp, Neisseria gonorrhoeae; identical to SEQ ID NO: 183

SEQ ID NO: 212 - DNA, 1047 bp, Neisseria gonorrhoeae; identical to SEQ ID NO: 206

SEQ ID NO: 213 - DNA, 1047 bp, Neisseria gonorrhoeae
atgaaaaaatccctgattgccctgactttggcagcccttcctgttgcggcaacggccgatgtcaccctgtacggcgccatcaaagccggcgtacaaacttaccgttctgtagaacatcgggaaggcaaagtaattggcgtgggaaccggcagcgaaatctccgacttcggttcaaaaatcggcttcaaaggccaagaagacctcggcaacggcctgaaagccatttggcagttggaacaaggcgcctccgtcgccggcactaacagcggctggggcaacaaacaatccttcatcggcttgaagggcggcttcggcaccatccgcgccggtagcctgaacagccccctgaaaaacaccaagaacaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcagggtcaaacggcgaatcttaccacgttggcttgaactaccgaaacaacggcttcttcgcacaatacgccggcttgttccaaagatacggcgaaggcactaaaaaaatggaatacgatggtcaaacttataatatccccggtttgtttgttgaaaaactgcaagttcaccgtttggtcggcggttacgacaataatgccctgtacgcctccgtagccgcacaacaacaagatgccaaattgtatggagcaatgagcggtaattcgcacaactctcaaaccgaagttgccgctaccgtagcataccgtttcggcaacgtaacgccccgtgtttcttacgcccacggcttcaaaggcactgttgatgatgcaaaccacgacaatacttacgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 214 - DNA, 1047 bp, Neisseria gonorrhoeae; identical to SEQ ID NO: 213

SEQ ID NO: 215 - DNA, 1050 bp, Neisseria gonorrhoeae
atgaaaaaatccctgattgccctgactttggcagcccttcctgttgcggcaacggccgatgttaccctgtacggcgccatcaaagccggcgtacaaacttaccgttctgtagaacatcgggaaggcaaagtagttggcgtgggaaccggcagcgaaatctccgacttcggttcaaaaatcggcttcaaaggtcaagaagacctcggcaacggcctgaaggccgtttggcagttggaacaaggcgcctccgtcgccggcactaacaccggctggggcaacaaacaatccttcgtcggcttgaaaggcggcttcggcaccatccgcgtcggcagcctgaacagccccctgaaaaacaccggtgccaacgtcaatgcttgggaatccggcaaatataccggcgagcttctggaaatcagcaaaatggccggacgggaacaccgctacctgtccgcacgctacgattctcccgaatttgccggcttcagcggcagcgtacaatacgcacctaaaggtaattcaggctcaaacggcgaatcttaccacgttggcttgaactaccgaaacagcggcttcttcgcacaatacgccggcttgttccaaagatacggcgaaggcactaaaaaaatcgaatactacgatgatcaaacttatagtatgcccagtctgtttgttgaaaaactgcaagttcaccgtttggtaggcggttacgacaataatgccctgtacgtttccgtagccgcacaacaacaagatgccaaattgtatggagcaacgagggttaattcgcacaactctcaaaccgaagttgccgctaccgcggcataccgtttcggcaatgtaacgccccgcgtttcttacgcccacggcttcaaaggcactgttgatgatgcaaaccacgacaatacttatgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 216 - DNA, 1047 bp, Neisseria gonorrhoeae; identical to SEQ ID NO: 213

SEQ ID NO: 217 - DNA, 1047 bp, Neisseria gonorrhoeae
atgaaaaaatccctgattgccctgactttggcagcccttcctgttgcggcaacggccgatgtcaccctgtacggcgccatcaaagccggcgtacaaacttaccgttctgtagaacatcgggaaggcaaagtaattggcgtgggaaccggcagcgaaatctccgacttcggttcaaaaatcggcttcaaaggccaagaagacctcggcaacggcctgaaagccatttggcagttggaacaaggcgcctccgtcgccggcactaacagcggctggggcaacaaacaatccttcatcggcttgaagggcggcttcggcaccatccgcgccggtagcctgaacagccccctgaaaaacaccaagaacaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcagggtcaaacggcgaatcttaccacgttggcttgaactaccgaaacaacggcttcttcgcacaatacgccggcttgttccaaagatacggcgaaggcactaaaaaaatggaatacgatggtcaaacttataatatccccagtttgtttgttgaaaaactgcaagttcaccgtttggtcggcggttacgacaataatgccctgtacgcctccgtagccgcacaacaacaagatgccaaattgtatgaagcaatgagcggtaattcgcacaactctcaaaccgaagttgccgctaccgtagcataccgtttcggcaacgtaacgccccgtgtttcttacgcccacggcttcaaaggcactgttgatgatgcaaaccacgacaatacttacgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 218 - DNA, 1047 bp, Neisseria gonorrhoeae; identical to SEQ ID NO: 181

SEQ ID NO: 219 - DNA, 1047 bp, Neisseria gonorrhoeae
atgaaaaaatccctgattgccctgactttggcagcccttcctgttgcggcaacggccgatgtcaccctgtacggcgccatcaaagccggcgtacaaacttaccgttctgtagaacatccagaaggcaaagtaattggcgtggaaaccggcagcgaaatctccgacttcggttcaaaaatcggcttcaaaggccaagaagacctcggcaacggcctgaaagccatttggcagttggaacaaggcgcctccgtcgccggcactaacagcggctggggcaacaaacaatccttcatcggcttgaagggcggcttcggcaccatccgcgccggtagcctgaacagccccctgaaaaacaccaagaacaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcagggtcaaacggcgaatcttaccacgttggcttgaactaccgaaacaacggcttcttcgcacaatacgccggcttgttccaaagatacggcgaaggcactaaaaaaatggaatacgatggtcaaacttataatatccccagtttgtttgttgaaaaactgcaagttcaccgtttggtcggcggttacgacaataatgccctgtacgcctccgtagccgcacaacaacaagatgccaaattgtatggagcaatgagcggtaattcgcacaactctcaaaccgaagttgccgctaccgtagcataccgtttcggcaacgtaacgccccgtgtttcttacgcccacggcttcaaaggcactgttgatgatgcaaaccacgacaatacttacgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 220 - DNA, 1047 bp, Neisseria gonorrhoeae; identical to SEQ ID NO: 181

SEQ ID NO: 221 - DNA, 1047 bp, Neisseria gonorrhoeae; identical to SEQ ID NO: 183

SEQ ID NO: 222 - DNA, 1047 bp, Neisseria gonorrhoeae; identical to SEQ ID NO: 183

SEQ ID NO: 223 - DNA, 1047 bp, Neisseria gonorrhoeae; identical to SEQ ID NO: 213

SEQ ID NO: 224 - DNA, 1047 bp, Neisseria gonorrhoeae; identical to SEQ ID NO: 178

SEQ ID NO: 225 - DNA, 1047 bp, Neisseria gonorrhoeae
atgaaaaaatccctgattgccctgactttggcagcccttcctgttgcggcaacggccgatgtcaccctgtacggcgccatcaaagccggcgtacaaacttaccgttctgtagaacatcgggaaggcaaagtaattggcgtggaaaccggcagcgaaatctccgacttcggttcaaaaatcggcttcaaaggccaagaagacctcggcaacggcctgaaagccatttggcagttggaacaaggcgcctccgtcgccggcactaacagcggctggggcaacaaacaatccttcatcggcttgaagggcggcttcggcaccatccgcgccggtagcctgaacagccccctgaaaaacaccaagaacaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcagggtcaaacggcgaatcttaccacgttggcttgaactaccgaaacaacggcttcttcgcacaatacgccggcttgttccaaagatacggcgaaggcactaaaaaaatcgaatacgatggtcaaacttataatatccccggtttgtttgttgaaaaactgcaagttcaccgtttggtcggcggttacgacaataatgccctgtacgcctccgtagccgcacaacaacaagatgccaaattgtatggagcaatgagcggtaattcgcacaactctcaaaccgaagttgccgctaccgtagcataccgtttcggcaacgtaacgccccgtgtttcttacgcccacggcttcaaaggcactgttgatgatgcaaaccacgacaatacttacgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 226 - DNA, 1047 bp, Neisseria gonorrhoeae
atgaaaaaatccctgattgccctgactttggcagcccttcctgttgcggcaacggccgatgtcaccctgtacggcgccatcaaagccggcgtacaaacttaccgttctgtagaacatcgggaaggcaaagtaattggcgtggaaaccggcagcgaaatctccgacttcggttcaaaaatcggcttcaaaggccaagaagacctcggcaacggcctgaaagccatttggcagttggaacaaggcgcctccgtcgccggcactaacagcggctggggcaacaaacaatccttcatcggcttgaagggcggcttcggcaccatccgcgccggtagcctgaacagccccctgaaaaacaccaagaacaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcagggtcaaacggcgaatcttaccacgttggcttgaactaccgaaacaacggcttcttcgcacaatacgccggcttgttccaaagatacggcgaaggcactaaaaaaatggaatacgatggtcaaacttataatatccccggtttgtttgttgaaaaactgcaagttcaccgtttggtcggcggttacgacaataatgccctgtacgcctccgtagccgcacaacaacaagatgccaaattgtatggagcaatgagcggtaattcgcacaactctcaaaccgaagttgccgctaccgtagcataccgtttcggcaacgtaacgccccgtgtttcttacgcccacggcttcaaaggcactgttgatgatgcaaaccacgacaatacttacgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 227 - DNA, 1047 bp, Neisseria gonorrhoeae; identical to SEQ ID NO: 213

SEQ ID NO: 228 - DNA, 72 bp, Neisseria gonorrhoeae
ctcgccatagttcaacggatagaacgtatgcctcctaagcgtaaaatacaggttcgattcctgttggcgagg

SEQ ID NO: 229 - DNA, 72 bp, Neisseria gonorrhoeae
cctcgccaacaggaatcgaacctgtattttacgcttaggaggcatacgttctatccgttgaactatggcgag

SEQ ID NO: 230 - RNA, 72 nt, Neisseria gonorrhoeae
cucgccauaguucaacggauagaacguaugccuccuaagcguaaaauacagguucgauuccuguuggcgagg

SEQ ID NO: 231 - DNA, 690 bp, Neisseria gonorrhoeae
gtgcaggcggatttagcctacgccgccgaacgcattacccacgattatccggaaccaaccggtgcaaaaaaaggcaaaataagcacggtaagcgattatttcagaaacatccgtacgcattccatccaccccagggtgtcggtcggctacgacttcggcggctggaggatagcggcagattatgcccgttacagaaagtggaacaacagtaaatattccgtcaacacaaaaaaggtgaacgaaaacaagggcgaaaagataaacgtgacgcaatatctgaaggcggaaaatcaggaaaacggtacgttccacgccgtttcttctctcggcttgtccgccgtttacgatttcaaactcaacgacaaattcaaaccctatatcggcatgcgcgtcggctacgggcacgtcagacatcaggttcgttcggttgaacaagaaaccacgactgttaccacttacctacagagtggtaagccaagtcctatcgtacgaggttcgaccctcaaacttccccatcacgaaagccgcagcagccgccgcttgggcttcggcgcgatggcgggcgtgggcatagacgtcgcgcccggtctgaccttggacgccggctaccgctaccactattggggacgcctggaaaacacccgcttcaaaacccacgaagcctcgttgggcgtgcgctaccgcttctga

SEQ ID NO: 232 - RNA, 690 nt, Neisseria gonorrhoeae
gugcaggcggauuuagccuacgccgccgaacgcauuacccacgauuauccggaaccaaccggugcaaaaaaaggcaaaauaagcacgguaagcgauuauuucagaaacauccguacgcauuccauccaccccagggugucggucggcuacgacuucggcggcuggaggauagcggcagauuaugcccguuacagaaaguggaacaacaguaaauauuccgucaacacaaaaaaggugaacgaaaacaagggcgaaaagauaaacgugacgcaauaucugaaggcggaaaaucaggaaaacgguacguuccacgccguuucuucucucggcuuguccgccguuuacgauuucaaacucaacgacaaauucaaacccuauaucggcaugcgcgucggcuacgggcacgucagacaucagguucguucgguugaacaagaaaccacgacuguuaccacuuaccuacagagugguaagccaaguccuaucguacgagguucgacccucaaacuuccccaucacgaaagccgcagcagccgccgcuugggcuucggcgcgauggcgggcgugggcauagacgucgcgcccggucugaccuuggacgccggcuaccgcuaccacuauuggggacgccuggaaaacacccgcuucaaaacccacgaagccucguugggcgugcgcuaccgcuucuga

SEQ ID NO: 233 - DNA, 690 bp, Neisseria gonorrhoeae
tcagaagcggtagcgcacgcccaacgaggcttcgtgggttttgaagcgggtgttttccaggcgtccccaatagtggtagcggtagccggcgtccaaggtcagaccgggcgcgacgtctatgcccacgcccgccatcgcgccgaagcccaagcggcggctgctgcggctttcgtgatggggaagtttgagggtcgaacctcgtacgataggacttggcttaccactctgtaggtaagtggtaacagtcgtggtttcttgttcaaccgaacgaacctgatgtctgacgtgcccgtagccgacgcgcatgccgatatagggtttgaatttgtcgttgagtttgaaatcgtaaacggcggacaagccgagagaagaaacggcgtggaacgtaccgttttcctgattttccgccttcagatattgcgtcacgtttatcttttcgcccttgttttcgttcaccttttttgtgttgacggaatatttactgttgttccactttctgtaacgggcataatctgccgctatcctccagccgccgaagtcgtagccgaccgacaccctggggtggatggaatgcgtacggatgtttctgaaataatcgcttaccgtgcttattttgcctttttttgcaccggttggttccggataatcgtgggtaatgcgttcggcggcgtaggctaaatccgcctgcac

SEQ ID NO: 234 - DNA, 798 bp, Neisseria gonorrhoeae
atgaatccagcccgcaaaaaaccttctcttctcttctcttctcttctcttctcttctcttctcttctcttcggcagcgcaggcggcaagtgaaggcaatggccgcggcccgtatgtgcaggcggatttagcctacgccgccgaacgcattacccacgattatccggaaccaaccgctccaggcaaaaacaaaataagcacggtaagcgattatttcagaaacatccgtacgcattccatccaccccagggtgtcggtcggctacgacttcggcggctggaggatagcggcagattatgcccgttacagaaagtggaacgacaataaatattccgtcgacataaaagagttggaaaacaagaatcagaataagagagacctgaagacggaaaatcaggaaaacggcagcttccacgccgtttcttctctcggcttatcagccgtttacgatttcaaactcaacgacaaattcaaaccctatatcggtgcgcgcgtcgcctacggacacgtcagacacagcatcgattcgactaaaaaaataacaggtactcttaccgcctaccctagtgatgctgacgcagcagttacggtttatcctgacggacatccgcaaaaaaacacctatcaaaaaagcaacagcagccgccgcttgggcttcggcgcgatggcgggcgtgggcatagacgtcgcgcccggcctgaccttggacgccggctaccgctaccacaactggggacgcttggaaaacacccgcttcaaaacccacgaagcctcattgggcatgcgctaccgcttctga

SEQ ID NO: 235 - RNA, 798 nt, Neisseria gonorrhoeae
augaauccagcccgcaaaaaaccuucucuucucuucucuucucuucucuucucuucucuucucuucucuucggcagcgcaggcggcaagugaaggcaauggccgcggcccguaugugcaggcggauuuagccuacgccgccgaacgcauuacccacgauuauccggaaccaaccgcuccaggcaaaaacaaaauaagcacgguaagcgauuauuucagaaacauccguacgcauuccauccaccccagggugucggucggcuacgacuucggcggcuggaggauagcggcagauuaugcccguuacagaaaguggaacgacaauaaauauuccgucgacauaaaagaguuggaaaacaagaaucagaauaagagagaccugaagacggaaaaucaggaaaacggcagcuuccacgccguuucuucucucggcuuaucagccguuuacgauuucaaacucaacgacaaauucaaacccuauaucggugcgcgcgucgccuacggacacgucagacacagcaucgauucgacuaaaaaaauaacagguacucuuaccgccuacccuagugaugcugacgcagcaguuacgguuuauccugacggacauccgcaaaaaaacaccuaucaaaaaagcaacagcagccgccgcuugggcuucggcgcgauggcgggcgugggcauagacgucgcgcccggccugaccuuggacgccggcuaccgcuaccacaacuggggacgcuuggaaaacacccgcuucaaaacccacgaagccucauugggcaugcgcuaccgcuucuga

SEQ ID NO: 236 - DNA, 798 bp, Neisseria gonorrhoeae
tcagaagcggtagcgcatgcccaatgaggcttcgtgggttttgaagcgggtgttttccaagcgtccccagttgtggtagcggtagccggcgtccaaggtcaggccgggcgcgacgtctatgcccacgcccgccatcgcgccgaagcccaagcggcggctgctgttgcttttttgataggtgtttttttgcggatgtccgtcaggataaaccgtaactgctgcgtcagcatcactagggtaggcggtaagagtacctgttatttttttagtcgaatcgatgctgtgtctgacgtgtccgtaggcgacgcgcgcaccgatatagggtttgaatttgtcgttgagtttgaaatcgtaaacggctgataagccgagagaagaaacggcgtggaagctgccgttttcctgattttccgtcttcaggtctctcttattctgattcttgttttccaactcttttatgtcgacggaatatttattgtcgttccactttctgtaacgggcataatctgccgctatcctccagccgccgaagtcgtagccgaccgacaccctggggtggatggaatgcgtacggatgtttctgaaataatcgcttaccgtgcttattttgtttttgcctggagcggttggttccggataatcgtgggtaatgcgttcggcggcgtaggctaaatccgcctgcacatacgggccgcggccattgccttcacttgccgcctgcgctgccgaagagaagagaagagaagagaagagaagagaagagaagagaagagaaggttttttgcgggctggattcat

SEQ ID NO: 237 - DNA, 828 bp, Neisseria gonorrhoeae
atgatgttgaaaaaattcgtactcggcggcattgccgcattggttttggcggcctgcggcggttcggaaggcggcagcggagcatcttccgcgcctgcacaatcggcaatttccggttctttaatcgagcgcatcaacaataaaggcacggttaccgtcggcacggaaggcacttacgcaccgtttacctaccacgacaaagacggcaaactgaccggttacgacgtggaagtaacccgcgccgtggcggaaaaactgggcgtaaaagtcgagtttaaagaaacgcaatgggattcgatgatggcgggtttgaaagccggacgtttcgacgtggtggcaaaccaagtcggcctgaccagccccgaacgccaggcgacatttgacaaatccgaaccttacagctggagcggtgcggttttggttgcgcataacgacagcaacattaaatccatagccgacatcaaaggcgtgaaaaccgcgcaatccctgaccagcaactacggcgaaaaagccaaagccgcaggtgcgcaactcgtgccggtggacggtttggcgcaatcgctgaccctgattgaacaaaaacgcgccgatgcgacgttgaacgatgaattggcggttttggactatctgaagaaaaacccgaatgcgggggtgaaaatcgtgtggtccgcgcctgccgatgaaaaagtcggttccggtctgattgtcaacaagggcaatgacgaggccgtggcgaaattcagcacggcaatcaacgagctgaaagccgacggcacgttgaaaaaactgggcgaacaattcttcggaaaagacatcagtgttcaataa

SEQ ID NO: 238 - RNA, 828 nt, Neisseria gonorrhoeae
augauguugaaaaaauucguacucggcggcauugccgcauugguuuuggcggccugcggcgguucggaaggcggcagcggagcaucuuccgcgccugcacaaucggcaauuuccgguucuuuaaucgagcgcaucaacaauaaaggcacgguuaccgucggcacggaaggcacuuacgcaccguuuaccuaccacgacaaagacggcaaacugaccgguuacgacguggaaguaacccgcgccguggcggaaaaacugggcguaaaagucgaguuuaaagaaacgcaaugggauucgaugauggcggguuugaaagccggacguuucgacgugguggcaaaccaagucggccugaccagccccgaacgccaggcgacauuugacaaauccgaaccuuacagcuggagcggugcgguuuugguugcgcauaacgacagcaacauuaaauccauagccgacaucaaaggcgugaaaaccgcgcaaucccugaccagcaacuacggcgaaaaagccaaagccgcaggugcgcaacucgugccgguggacgguuuggcgcaaucgcugacccugauugaacaaaaacgcgccgaugcgacguugaacgaugaauuggcgguuuuggacuaucugaagaaaaacccgaaugcgggggugaaaaucgugugguccgcgccugccgaugaaaaagucgguuccggucugauugucaacaagggcaaugacgaggccguggcgaaauucagcacggcaaucaacgagcugaaagccgacggcacguugaaaaaacugggcgaacaauucuucggaaaagacaucaguguucaauaa

SEQ ID NO: 239 - DNA, 828 bp, Neisseria gonorrhoeae
ttattgaacactgatgtcttttccgaagaattgttcgcccagttttttcaacgtgccgtcggctttcagctcgttgattgccgtgctgaatttcgccacggcctcgtcattgcccttgttgacaatcagaccggaaccgactttttcatcggcaggcgcggaccacacgattttcacccccgcattcgggtttttcttcagatagtccaaaaccgccaattcatcgttcaacgtcgcatcggcgcgtttttgttcaatcagggtcagcgattgcgccaaaccgtccaccggcacgagttgcgcacctgcggctttggctttttcgccgtagttgctggtcagggattgcgcggttttcacgcctttgatgtcggctatggatttaatgttgctgtcgttatgcgcaaccaaaaccgcaccgctccagctgtaaggttcggatttgtcaaatgtcgcctggcgttcggggctggtcaggccgacttggtttgccaccacgtcgaaacgtccggctttcaaacccgccatcatcgaatcccattgcgtttctttaaactcgacttttacgcccagtttttccgccacggcgcgggttacttccacgtcgtaaccggtcagtttgccgtctttgtcgtggtaggtaaacggtgcgtaagtgccttccgtgccgacggtaaccgtgcctttattgttgatgcgctcgattaaagaaccggaaattgccgattgtgcaggcgcggaagatgctccgctgccgccttccgaaccgccgcaggccgccaaaaccaatgcggcaatgccgccgagtacgaattttttcaacatcat

SEQ ID NO: 240 - DNA, 756 bp, Neisseria gonorrhoeae
atgattaaaatccgcaatatccataagacctttggcgaaaacaccattttgcgcggcatcgatttggatgtgggcaaagggcaggtggtcgtcatcctcgggccttccggctcgggtaaaacaacatttctgcgctgcctaaacgcgttggaaatgcccgaagacggacaaatcgagttcgacaacgcgcggccgttacgcattgatttttccaaaaaaacaagcaaacacgatattttggcactgcgccgcaagtccggaatggtattccaacaatacaacctcttcccgcataaaaccgtgttggaaaacgtgatggaagggccggttgccgtacagggcaagcctgccgcccaagcgcgcgaagaggctttgaaactgctggaaaaagtcggcttgggcgataaagtggatttgtatccctaccagctttccggcggtcagcagcagcgtgtcggtatcgcccgcgcactggcgattcagcctgaattgatgctgtttgacgaacccacttccgcgctggaccccgagttggtgcaagacgtgttggacgccatgaaggaattggcgcgggaaggttggacgatggtcgtcgttacccacgaaatcaagttcacgctggaagttgccacgaacgtcgtcgtgatggacggcggcgttatcgtagagcagggcagcccgaaagagttgttcgaccacctcaaacacgaacggacgcggagatttttaagccaaatccaatctgccaagatttga

SEQ ID NO: 241 - RNA, 756 nt, Neisseria gonorrhoeae
augauuaaaauccgcaauauccauaagaccuuuggcgaaaacaccauuuugcgcggcaucgauuuggaugugggcaaagggcagguggucgucauccucgggccuuccggcucggguaaaacaacauuucugcgcugccuaaacgcguuggaaaugcccgaagacggacaaaucgaguucgacaacgcgcggccguuacgcauugauuuuuccaaaaaaacaagcaaacacgauauuuuggcacugcgccgcaaguccggaaugguauuccaacaauacaaccucuucccgcauaaaaccguguuggaaaacgugauggaagggccgguugccguacagggcaagccugccgcccaagcgcgcgaagaggcuuugaaacugcuggaaaaagucggcuugggcgauaaaguggauuuguaucccuaccagcuuuccggcggucagcagcagcgugucgguaucgcccgcgcacuggcgauucagccugaauugaugcuguuugacgaacccacuuccgcgcuggaccccgaguuggugcaagacguguuggacgccaugaaggaauuggcgcgggaagguuggacgauggucgucguuacccacgaaaucaaguucacgcuggaaguugccacgaacgucgucgugauggacggcggcguuaucguagagcagggcagcccgaaagaguuguucgaccaccucaaacacgaacggacgcggagauuuuuaagccaaauccaaucugccaagauuuga

SEQ ID NO: 242 - DNA, 756 bp, Neisseria gonorrhoeae
tcaaatcttggcagattggatttggcttaaaaatctccgcgtccgttcgtgtttgaggtggtcgaacaactctttcgggctgccctgctctacgataacgccgccgtccatcacgacgacgttcgtggcaacttccagcgtgaacttgatttcgtgggtaacgacgaccatcgtccaaccttcccgcgccaattccttcatggcgtccaacacgtcttgcaccaactcggggtccagcgcggaagtgggttcgtcaaacagcatcaattcaggctgaatcgccagtgcgcgggcgataccgacacgctgctgctgaccgccggaaagctggtagggatacaaatccactttatcgcccaagccgactttttccagcagtttcaaagcctcttcgcgcgcttgggcggcaggcttgccctgtacggcaaccggcccttccatcacgttttccaacacggttttatgcgggaagaggttgtattgttggaataccattccggacttgcggcgcagtgccaaaatatcgtgtttgcttgtttttttggaaaaatcaatgcgtaacggccgcgcgttgtcgaactcgatttgtccgtcttcgggcatttccaacgcgtttaggcagcgcagaaatgttgttttacccgagccggaaggcccgaggatgacgaccacctgccctttgcccacatccaaatcgatgccgcgcaaaatggtgttttcgccaaaggtcttatggatattgcggattttaatcat

SEQ ID NO: 243 - DNA, 840 bp, Neisseria gonorrhoeae
gtgaaacgcatttttctgtttttggctaccaatatcgctgttttggtcgtaatcaacattgttttggcggttctgggcatcaacagccggggcggcgcgggcagcctgttggcgtattccgccgtcgtcggcttcactggttcgattatttcgctgctgatgtccaaatttatcgccaaacaatcggtcggtgcggaagtcatcgacacgccgcgcaccgaagaagaagcctggcttctgaacactgtcgaagcccaagcgcggcaatggaatctgaaaacgccagaagtcgccatctaccactcccccgaacccaatgcctttgccacgggcgcatcgagaaacagctccctgatcgccgtcagcaccggtttgctcgaccatatgacgcgcgacgaagtggaagccgtgttggcgcacgaaatggcgcacgtcggcaacggcgacatggttacgctgacgctgattcaaggcgtggtcaatacctttgtcgtgttcctgtcgcgcattattgccaacctgattgcccgaaacaacgacggcagccagtcccagggaacttatttcctagtcagcatggtattccaaatcctgttcggcttccttgccagcctgattgtcatgtggttcagccgccaacgcgaataccgcgccgacgcgggcgcggcaaaactggtcggcgcaccgaaaatgatttccgccctgcaaaggcttaaaggcaacccggtcgatttgcccgaagaaatgaacgcaatgggcatcgccggagatacgcgcgactccctgctcagcacccacccttcgctggacaaccgaatcgcccgcctcaaatcgctttaa

SEQ ID NO: 244 - RNA, 840 nt, Neisseria gonorrhoeae
gugaaacgcauuuuucuguuuuuggcuaccaauaucgcuguuuuggucguaaucaacauuguuuuggcgguucugggcaucaacagccggggcggcgcgggcagccuguuggcguauuccgccgucgucggcuucacugguucgauuauuucgcugcugauguccaaauuuaucgccaaacaaucggucggugcggaagucaucgacacgccgcgcaccgaagaagaagccuggcuucugaacacugucgaagcccaagcgcggcaauggaaucugaaaacgccagaagucgccaucuaccacucccccgaacccaaugccuuugccacgggcgcaucgagaaacagcucccugaucgccgucagcaccgguuugcucgaccauaugacgcgcgacgaaguggaagccguguuggcgcacgaaauggcgcacgucggcaacggcgacaugguuacgcugacgcugauucaaggcguggucaauaccuuugucguguuccugucgcgcauuauugccaaccugauugcccgaaacaacgacggcagccagucccagggaacuuauuuccuagucagcaugguauuccaaauccuguucggcuuccuugccagccugauugucaugugguucagccgccaacgcgaauaccgcgccgacgcgggcgcggcaaaacuggucggcgcaccgaaaaugauuuccgcccugcaaaggcuuaaaggcaacccggucgauuugcccgaagaaaugaacgcaaugggcaucgccggagauacgcgcgacucccugcucagcacccacccuucgcuggacaaccgaaucgcccgccucaaaucgcuuuaa

SEQ ID NO: 245 - DNA, 840 bp, Neisseria gonorrhoeae
ttaaagcgatttgaggcgggcgattcggttgtccagcgaagggtgggtgctgagcagggagtcgcgcgtatctccggcgatgcccattgcgttcatttcttcgggcaaatcgaccgggttgcctttaagcctttgcagggcggaaatcattttcggtgcgccgaccagttttgccgcgcccgcgtcggcgcggtattcgcgttggcggctgaaccacatgacaatcaggctggcaaggaagccgaacaggatttggaataccatgctgactaggaaataagttccctgggactggctgccgtcgttgtttcgggcaatcaggttggcaataatgcgcgacaggaacacgacaaaggtattgaccacgccttgaatcagcgtcagcgtaaccatgtcgccgttgccgacgtgcgccatttcgtgcgccaacacggcttccacttcgtcgcgcgtcatatggtcgagcaaaccggtgctgacggcgatcagggagctgtttctcgatgcgcccgtggcaaaggcattgggttcgggggagtggtagatggcgacttctggcgttttcagattccattgccgcgcttgggcttcgacagtgttcagaagccaggcttcttcttcggtgcgcggcgtgtcgatgacttccgcaccgaccgattgtttggcgataaatttggacatcagcagcgaaataatcgaaccagtgaagccgacgacggcggaatacgccaacaggctgcccgcgccgccccggctgttgatgcccagaaccgccaaaacaatgttgattacgaccaaaacagcgatattggtagccaaaaacagaaaaatgcgtttcac

SEQ ID NO: 246 - DNA, 612 bp, Neisseria gonorrhoeae
atgaagaataatgattgcttgcgcctgaaaaatccccagtccggtatggcgttgatagaagtcttggtcgctatgctcgttctgaccatcggtattttggcattgctgtccgtacagttgcggacagtcgcttccgtcagggaggcggaaacgcaaaccatcgtcagccaaatcacgcaaaacctgatggaaggaatgttgatgaatccgaccattgatttggacagcaacaagaaaaactatagtctttacatgggaaaacagacactatcagctgtggatggtgagtttatgcttgatgccgagaaaagtaaggcgcagttggcagaggaacaattgaagagatttagtcatgagctgaaaaatgccttgccggatgcggtagctattcattacgccgtctgcaaggattcgtcgggtgacgcgccgacattgtccgacagcggtgctttttcttcaaattgcgacaataaggcaaacggggatactttgattaaagtattgtgggtaaatgattcggcaggggattcggatatttcccgtacgaatcttgaagtgagcggcgacaatatcgtatatacctatcaggcaagggtcggaggtcgtgaatga

SEQ ID NO: 247 - RNA, 612 nt, Neisseria gonorrhoeae
augaagaauaaugauugcuugcgccugaaaaauccccaguccgguauggcguugauagaagucuuggucgcuaugcucguucugaccaucgguauuuuggcauugcuguccguacaguugcggacagucgcuuccgucagggaggcggaaacgcaaaccaucgucagccaaaucacgcaaaaccugauggaaggaauguugaugaauccgaccauugauuuggacagcaacaagaaaaacuauagucuuuacaugggaaaacagacacuaucagcuguggauggugaguuuaugcuugaugccgagaaaaguaaggcgcaguuggcagaggaacaauugaagagauuuagucaugagcugaaaaaugccuugccggaugcgguagcuauucauuacgccgucugcaaggauucgucgggugacgcgccgacauuguccgacagcggugcuuuuucuucaaauugcgacaauaaggcaaacggggauacuuugauuaaaguauuguggguaaaugauucggcaggggauucggauauuucccguacgaaucuugaagugagcggcgacaauaucguauauaccuaucaggcaagggucggaggucgugaauga

SEQ ID NO: 248 - DNA, 612 bp, Neisseria gonorrhoeae
tcattcacgacctccgacccttgcctgataggtatatacgatattgtcgccgctcacttcaagattcgtacgggaaatatccgaatcccctgccgaatcatttacccacaatactttaatcaaagtatccccgtttgccttattgtcgcaatttgaagaaaaagcaccgctgtcggacaatgtcggcgcgtcacccgacgaatccttgcagacggcgtaatgaatagctaccgcatccggcaaggcatttttcagctcatgactaaatctcttcaattgttcctctgccaactgcgccttacttttctcggcatcaagcataaactcaccatccacagctgatagtgtctgttttcccatgtaaagactatagtttttcttgttgctgtccaaatcaatggtcggattcatcaacattccttccatcaggttttgcgtgatttggctgacgatggtttgcgtttccgcctccctgacggaagcgactgtccgcaactgtacggacagcaatgccaaaataccgatggtcagaacgagcatagcgaccaagacttctatcaacgccataccggactggggatttttcaggcgcaagcaatcattattcttcat

SEQ ID NO: 249 - DNA, 1797 bp, Neisseria gonorrhoeae
atgcgctacaaaccccttctgcttgccctgatgctcgttttttccacgcccgccgttgccgcccacgacgcggcacacaaccgttccgccgaagtgaaaaaacaggcgaagaacaaaaaagaacagcccgaagcggcggaaggcaaaaaagaaaaaggcaaaaatgccgcagtgaaagataaaaaaacaggcggcaaagaggcggcaaaagagttcaaaaaaaccgccaaaaaccgcaaagaagcagagaaggaggcgacatccaggcagtctgcgcgcaaaggacgcgaaggggataaggaatcgaaggcggaacacaaaaaggcacatggcaagcccgtgtccggatccaaagaaaaaaacgcaaaaacacagcctgaaaacaaacaaggcaaaaaaggggcaaaaggacagggcaatccgcgcaagggcggcaaggcggaaaaagacactgtttctgcaaataaaaaagcccgttccgacaagaacggcaaagcagtgaaacaggacaaaaaacacacggaagagaaaaatgccaaaaccgattccgacgaattgaaagccgccgttgccgctgccaccaatgatgtcgaaaacaaaaaagccctgctcaaacaaagcgaaggaatgctgcttcatgtcagcaattccctcaaacagcttcaggaagagcgtatccgccaagaacgtatccgccaagagcgtatccgtcaggcgcgcggcaaccttgcttccgtcaaccgcaaacagcgcgaggcttgggacaaattccaaaaactcaataccgagctgaaccgtttgaaaacggaagtcgccgctacgaaagcgcagatttcccgtttcgtatcggggaactataaaaacagccggccgaatgcggttgccctgttcctgaaaaacgccgaaccgggtcagaaaaaccgctttttgcgttatacgcgttatgtaaacgcctccaatcgggaagttgtcaaggatttggaaaaacagcagaaggctttggcggtacaagagcagaaaatcaacaatgagcttgcccgtttgaagaaaattcaggcaaacgtgcaatccctgctgaaaaaacagggtgtaaccgatgcggcggaacagacggaaagccgcagacagaatgccaaaatctccaaagatgcccgaaaactgctggaacagaaagggaacgagcagcagctgaacaagctcttgagcaatttggagaaaaaaaaagccgaacaccgcattcaggatgcggaagcaaaaagaaaattggctgaagccaaactggcggcagccgaaaaagccagaaaagaagcggcgcagcagaaggctgaagcgcgacgtgcggaaatgtccaacctgaccgccgaagacaggaacatccaagcgccttcggttatgggtatcggcagtgccgacggtttcagccgcatgcagggacgtttgaaaaaaccggttgacggtgtgccgaccgggcttttcgggcagaaccggagcggcggcgatgtttggaaaggcgtgttctattccactgcgcctgcaacggttgaaagcattgcgccgggaacggtaagctatgcggacgagttggacggctacggcaaagtggtcgtgatcgatcacggcgagaactacatcagcatctatgccggtttgagcgaaatttccgccggcaagggttatacggtcgcggcaggaagcaaaatcggcacgagcgggtcgctgccggacggggaagaggggctttacctgcaaatacgttatcgaggtcaggtgttgaacccttcgggctggatacgttga

SEQ ID NO: 250 - RNA, 1797 nt, Neisseria gonorrhoeae
augcgcuacaaaccccuucugcuugcccugaugcucguuuuuuccacgcccgccguugccgcccacgacgcggcacacaaccguuccgccgaagugaaaaaacaggcgaagaacaaaaaagaacagcccgaagcggcggaaggcaaaaaagaaaaaggcaaaaaugccgcagugaaagauaaaaaaacaggcggcaaagaggcggcaaaagaguucaaaaaaaccgccaaaaaccgcaaagaagcagagaaggaggcgacauccaggcagucugcgcgcaaaggacgcgaaggggauaaggaaucgaaggcggaacacaaaaaggcacauggcaagcccguguccggauccaaagaaaaaaacgcaaaaacacagccugaaaacaaacaaggcaaaaaaggggcaaaaggacagggcaauccgcgcaagggcggcaaggcggaaaaagacacuguuucugcaaauaaaaaagcccguuccgacaagaacggcaaagcagugaaacaggacaaaaaacacacggaagagaaaaaugccaaaaccgauuccgacgaauugaaagccgccguugccgcugccaccaaugaugucgaaaacaaaaaagcccugcucaaacaaagcgaaggaaugcugcuucaugucagcaauucccucaaacagcuucaggaagagcguauccgccaagaacguauccgccaagagcguauccgucaggcgcgcggcaaccuugcuuccgucaaccgcaaacagcgcgaggcuugggacaaauuccaaaaacucaauaccgagcugaaccguuugaaaacggaagucgccgcuacgaaagcgcagauuucccguuucguaucggggaacuauaaaaacagccggccgaaugcgguugcccuguuccugaaaaacgccgaaccgggucagaaaaaccgcuuuuugcguuauacgcguuauguaaacgccuccaaucgggaaguugucaaggauuuggaaaaacagcagaaggcuuuggcgguacaagagcagaaaaucaacaaugagcuugcccguuugaagaaaauucaggcaaacgugcaaucccugcugaaaaaacaggguguaaccgaugcggcggaacagacggaaagccgcagacagaaugccaaaaucuccaaagaugcccgaaaacugcuggaacagaaagggaacgagcagcagcugaacaagcucuugagcaauuuggagaaaaaaaaagccgaacaccgcauucaggaugcggaagcaaaaagaaaauuggcugaagccaaacuggcggcagccgaaaaagccagaaaagaagcggcgcagcagaaggcugaagcgcgacgugcggaaauguccaaccugaccgccgaagacaggaacauccaagcgccuucgguuauggguaucggcagugccgacgguuucagccgcaugcagggacguuugaaaaaaccgguugacggugugccgaccgggcuuuucgggcagaaccggagcggcggcgauguuuggaaaggcguguucuauuccacugcgccugcaacgguugaaagcauugcgccgggaacgguaagcuaugcggacgaguuggacggcuacggcaaaguggucgugaucgaucacggcgagaacuacaucagcaucuaugccgguuugagcgaaauuuccgccggcaaggguuauacggucgcggcaggaagcaaaaucggcacgagcgggucgcugccggacggggaagaggggcuuuaccugcaaauacguuaucgaggucagguguugaacccuucgggcuggauacguuga

SEQ ID NO: 251 - DNA, 1797 bp, Neisseria gonorrhoeae
tcaacgtatccagcccgaagggttcaacacctgacctcgataacgtatttgcaggtaaagcccctcttccccgtccggcagcgacccgctcgtgccgattttgcttcctgccgcgaccgtataacccttgccggcggaaatttcgctcaaaccggcatagatgctgatgtagttctcgccgtgatcgatcacgaccactttgccgtagccgtccaactcgtccgcatagcttaccgttcccggcgcaatgctttcaaccgttgcaggcgcagtggaatagaacacgcctttccaaacatcgccgccgctccggttctgcccgaaaagcccggtcggcacaccgtcaaccggttttttcaaacgtccctgcatgcggctgaaaccgtcggcactgccgatacccataaccgaaggcgcttggatgttcctgtcttcggcggtcaggttggacatttccgcacgtcgcgcttcagccttctgctgcgccgcttcttttctggctttttcggctgccgccagtttggcttcagccaattttctttttgcttccgcatcctgaatgcggtgttcggctttttttttctccaaattgctcaagagcttgttcagctgctgctcgttccctttctgttccagcagttttcgggcatctttggagattttggcattctgtctgcggctttccgtctgttccgccgcatcggttacaccctgttttttcagcagggattgcacgtttgcctgaattttcttcaaacgggcaagctcattgttgattttctgctcttgtaccgccaaagccttctgctgtttttccaaatccttgacaacttcccgattggaggcgtttacataacgcgtataacgcaaaaagcggtttttctgacccggttcggcgtttttcaggaacagggcaaccgcattcggccggctgtttttatagttccccgatacgaaacgggaaatctgcgctttcgtagcggcgacttccgttttcaaacggttcagctcggtattgagtttttggaatttgtcccaagcctcgcgctgtttgcggttgacggaagcaaggttgccgcgcgcctgacggatacgctcttggcggatacgttcttggcggatacgctcttcctgaagctgtttgagggaattgctgacatgaagcagcattccttcgctttgtttgagcagggcttttttgttttcgacatcattggtggcagcggcaacggcggctttcaattcgtcggaatcggttttggcatttttctcttccgtgtgttttttgtcctgtttcactgctttgccgttcttgtcggaacgggcttttttatttgcagaaacagtgtctttttccgccttgccgcccttgcgcggattgccctgtccttttgccccttttttgccttgtttgttttcaggctgtgtttttgcgtttttttctttggatccggacacgggcttgccatgtgcctttttgtgttccgccttcgattccttatccccttcgcgtcctttgcgcgcagactgcctggatgtcgcctccttctctgcttctttgcggtttttggcggtttttttgaactcttttgccgcctctttgccgcctgtttttttatctttcactgcggcatttttgcctttttcttttttgccttccgccgcttcgggctgttcttttttgttcttcgcctgttttttcacttcggcggaacggttgtgtgccgcgtcgtgggcggcaacggcgggcgtggaaaaaacgagcatcagggcaagcagaaggggtttgtagcgcat

SEQ ID NO: 252 - DNA, 321 bp, Neisseria gonorrhoeae
atgattacccttaccgagaatgccgcaaaacacatcaatgactatctcgccaaacgcggcaaaggcttgggcgtacgcttgggtgtaaaaaccagcggctgctcggggatggcgtacaaccttgaatttgtcgatgaagccaacggcgacgacctgattttcgaaggacacggcgcgcgcatttatatcgacccgaaaagcttggtttatctggacggcacacaagtcgattacaccaaagaagatttgcaggaaggtttcaaatttgaaaaccccaatgtcaaagactcctgcggctgcggcgagagcttccacgtttaa

SEQ ID NO: 253 - RNA, 321 nt, Neisseria gonorrhoeae
augauuacccuuaccgagaaugccgcaaaacacaucaaugacuaucucgccaaacgcggcaaaggcuugggcguacgcuuggguguaaaaaccagcggcugcucggggauggcguacaaccuugaauuugucgaugaagccaacggcgacgaccugauuuucgaaggacacggcgcgcgcauuuauaucgacccgaaaagcuugguuuaucuggacggcacacaagucgauuacaccaaagaagauuugcaggaagguuucaaauuugaaaaccccaaugucaaagacuccugcggcugcggcgagagcuuccacguuuaa

SEQ ID NO: 254 - DNA, 321 bp, Neisseria gonorrhoeae
ttaaacgtggaagctctcgccgcagccgcaggagtctttgacattggggttttcaaatttgaaaccttcctgcaaatcttctttggtgtaatcgacttgtgtgccgtccagataaaccaagcttttcgggtcgatataaatgcgcgcgccgtgtccttcgaaaatcaggtcgtcgccgttggcttcatcgacaaattcaaggttgtacgccatccccgagcagccgctggtttttacacccaagcgtacgcccaagcctttgccgcgtttggcgagatagtcattgatgtgttttgcggcattctcggtaagggtaatcat

SEQ ID NO: 255 - DNA, 387 bp, Neisseria gonorrhoeae
atggcatacagcgataaagtaatcgaccactacgaaaatccccgcaacgtcggcactttcgacaaaaacgacgagtccgtcggcaccggcatggtcggcgcgcccgcctgcggcgacgtgatgcgcctgcaaatcaaagtgaacgatgaaggcatcatcgaagatgcgaaattcaaaacttacggctgcggttccgccatcgcttcgtccagcctgattaccgagtgggtcaaaggcaaaagtctggatgacgcgctggcaatcaaaaacagcgaaatcgccgaagaactggaattgccgccggtaaaaatccactgctccatcttggctgaagatgcggtaaaagcggccgttgccgactaccgcaaacgtcaggaaaacagataa

SEQ ID NO: 256 - RNA, 387 nt, Neisseria gonorrhoeae
auggcauacagcgauaaaguaaucgaccacuacgaaaauccccgcaacgucggcacuuucgacaaaaacgacgaguccgucggcaccggcauggucggcgcgcccgccugcggcgacgugaugcgccugcaaaucaaagugaacgaugaaggcaucaucgaagaugcgaaauucaaaacuuacggcugcgguuccgccaucgcuucguccagccugauuaccgagugggucaaaggcaaaagucuggaugacgcgcuggcaaucaaaaacagcgaaaucgccgaagaacuggaauugccgccgguaaaaauccacugcuccaucuuggcugaagaugcgguaaaagcggccguugccgacuaccgcaaacgucaggaaaacagauaa

SEQ ID NO: 257 - DNA, 387 bp, Neisseria gonorrhoeae
ttatctgttttcctgacgtttgcggtagtcggcaacggccgcttttaccgcatcttcagccaagatggagcagtggatttttaccggcggcaattccagttcttcggcgatttcgctgtttttgattgccagcgcgtcatccagacttttgcctttgacccactcggtaatcaggctggacgaagcgatggcggaaccgcagccgtaagttttgaatttcgcatcttcgatgatgccttcatcgttcactttgatttgcaggcgcatcacgtcgccgcaggcgggcgcgccgaccatgccggtgccgacggactcgtcgtttttgtcgaaagtgccgacgttgcggggattttcgtagtggtcgattactttatcgctgtatgccat

SEQ ID NO: 258 - DNA, 255 bp, Neisseria gonorrhoeae
atgaacaaacttttcgttaccgccctgtccgccctcgccttgtccgcctgcgccggcacttggcagggcgcgaaacaagacaccgcccgcaaccttgacaaaacacaggccgccgccgaacgcgccgccgaacaaacaggcaacgccgtcgaaaaaggttgggacaaaaccaaagaagccgtcaaaaaaggcggcaatgccgtcggacgcggcatttcccatctcggcaaaaaaatcgaaaacgccaccgaataa

SEQ ID NO: 259 - RNA, 255 nt, Neisseria gonorrhoeae
augaacaaacuuuucguuaccgcccuguccgcccucgccuuguccgccugcgccggcacuuggcagggcgcgaaacaagacaccgcccgcaaccuugacaaaacacaggccgccgccgaacgcgccgccgaacaaacaggcaacgccgucgaaaaagguugggacaaaaccaaagaagccgucaaaaaaggcggcaaugccgucggacgcggcauuucccaucucggcaaaaaaaucgaaaacgccaccgaauaa

SEQ ID NO: 260 - DNA, 255 bp, Neisseria gonorrhoeae
ttattcggtggcgttttcgatttttttgccgagatgggaaatgccgcgtccgacggcattgccgccttttttgacggcttctttggttttgtcccaacctttttcgacggcgttgcctgtttgttcggcggcgcgttcggcggcggcctgtgttttgtcaaggttgcgggcggtgtcttgtttcgcgccctgccaagtgccggcgcaggcggacaaggcgagggcggacagggcggtaacgaaaagtttgttcat

SEQ ID NO: 261 - DNA, 738 bp, Neisseria gonorrhoeae
atggctttgcaagatcgtaccggtcaaaaagtaccttccgtagtattccgcacccgcgtcggcgacacttggaaagatgtgtctaccgatgatttgttcaaaggcaaaaaagtagtcgtattctccctgcccggtgcatttaccccgacttgttcttcttcacacctgccgcgttacaacgaattgttcggcgcgttcaaagaaaacggcgttgacgcaatctgctgcgtatctgtaaacgatactttcgtaatgaacgcttgggctgccgaagaagaatcagacaacatctacatgattcctgacggcaacggcgaatttaccgaaggtatgggtatgctggtcggtaaagaagacttgggcttcggcaaacgctcttggcgttactccatgctggttaacgacggcgtggttgaaaaaatgttcatcgaacctgaagaaccgggcgatcctttcaaagtatccgatgcagatactatgctgaaattcgttgctcccgattggaaggctcaagagtctgtggcaattttcactaaaccaggttgccaattctgtgccaaagtcaaacaagctttgcaagacaaaggtttgtcttacgaagaaatcgtattgggcaaagatgcaaccgttacttccgttcgcgctattaccggcaagatgactgcccctcaagtcttcatcggcggcaaatacatcggcggcagcgaagatttggaagcttacttggctaaaaactga

SEQ ID NO: 262 - RNA, 738 nt, Neisseria gonorrhoeae
auggcuuugcaagaucguaccggucaaaaaguaccuuccguaguauuccgcacccgcgucggcgacacuuggaaagaugugucuaccgaugauuuguucaaaggcaaaaaaguagucguauucucccugcccggugcauuuaccccgacuuguucuucuucacaccugccgcguuacaacgaauuguucggcgcguucaaagaaaacggcguugacgcaaucugcugcguaucuguaaacgauacuuucguaaugaacgcuugggcugccgaagaagaaucagacaacaucuacaugauuccugacggcaacggcgaauuuaccgaagguauggguaugcuggucgguaaagaagacuugggcuucggcaaacgcucuuggcguuacuccaugcugguuaacgacggcgugguugaaaaaauguucaucgaaccugaagaaccgggcgauccuuucaaaguauccgaugcagauacuaugcugaaauucguugcucccgauuggaaggcucaagagucuguggcaauuuucacuaaaccagguugccaauucugugccaaagucaaacaagcuuugcaagacaaagguuugucuuacgaagaaaucguauugggcaaagaugcaaccguuacuuccguucgcgcuauuaccggcaagaugacugccccucaagucuucaucggcggcaaauacaucggcggcagcgaagauuuggaagcuuacuuggcuaaaaacuga

SEQ ID NO: 263 - DNA, 738 bp, Neisseria gonorrhoeae
tcagtttttagccaagtaagcttccaaatcttcgctgccgccgatgtatttgccgccgatgaagacttgaggggcagtcatcttgccggtaatagcgcgaacggaagtaacggttgcatctttgcccaatacgatttcttcgtaagacaaacctttgtcttgcaaagcttgtttgactttggcacagaattggcaacctggtttagtgaaaattgccacagactcttgagccttccaatcgggagcaacgaatttcagcatagtatctgcatcggatactttgaaaggatcgcccggttcttcaggttcgatgaacattttttcaaccacgccgtcgttaaccagcatggagtaacgccaagagcgtttgccgaagcccaagtcttctttaccgaccagcatacccataccttcggtaaattcgccgttgccgtcaggaatcatgtagatgttgtctgattcttcttcggcagcccaagcgttcattacgaaagtatcgtttacagatacgcagcagattgcgtcaacgccgttttctttgaacgcgccgaacaattcgttgtaacgcggcaggtgtgaagaagaacaagtcggggtaaatgcaccgggcagggagaatacgactacttttttgcctttgaacaaatcatcggtagacacatctttccaagtgtcgccgacgcgggtgcggaatactacggaaggtactttttgaccggtacgatcttgcaaagccat

SEQ ID NO: 264 - DNA, 561 bp, Neisseria gonorrhoeae
atgaaaacagcacaagaactgcgcgccggcaatgtatttatggtcggcaacgatcctatggtcgttcaaaaaaccgaatacatcaaaggcggccgctcttccgccaaagtcagcatgaaactgaaaaacctgctgaccggcgctgcttccgaaaccatttacaaagccgacgacaaattcgacgtggtcatcctgtcccgcaaaaactgtacgtacagctattttgccgacccgatgtacgtctttatggacgaagaattcaaccaatacgaaatcgaagccgacaacatcggcgacgcgttgaaattcatcgttgacggtatggaagaccaatgcgaagttaccttctatgaaggcaatcccatttctgtcgaactgcccaccatcatcgtgcgcgaagtcgagtacaccgagcctgccgtcaaaggcgatacttccggcaaagtgatgaaaaccgcgcgtctggtcggcggcaccgaaatccaagtgatgtcttacatcgaaaacggcgacaaagtcgaaatcgatacccgtaccggcgaattccgcaaacgcgcctga

SEQ ID NO: 265 - RNA, 561 nt, Neisseria gonorrhoeae
augaaaacagcacaagaacugcgcgccggcaauguauuuauggucggcaacgauccuauggucguucaaaaaaccgaauacaucaaaggcggccgcucuuccgccaaagucagcaugaaacugaaaaaccugcugaccggcgcugcuuccgaaaccauuuacaaagccgacgacaaauucgacguggucauccugucccgcaaaaacuguacguacagcuauuuugccgacccgauguacgucuuuauggacgaagaauucaaccaauacgaaaucgaagccgacaacaucggcgacgcguugaaauucaucguugacgguauggaagaccaaugcgaaguuaccuucuaugaaggcaaucccauuucugucgaacugcccaccaucaucgugcgcgaagucgaguacaccgagccugccgucaaaggcgauacuuccggcaaagugaugaaaaccgcgcgucuggucggcggcaccgaaauccaagugaugucuuacaucgaaaacggcgacaaagucgaaaucgauacccguaccggcgaauuccgcaaacgcgccuga

SEQ ID NO: 266 - DNA, 561 bp, Neisseria gonorrhoeae
tcaggcgcgtttgcggaattcgccggtacgggtatcgatttcgactttgtcgccgttttcgatgtaagacatcacttggatttcggtgccgccgaccagacgcgcggttttcatcactttgccggaagtatcgcctttgacggcaggctcggtgtactcgacttcgcgcacgatgatggtgggcagttcgacagaaatgggattgccttcatagaaggtaacttcgcattggtcttccataccgtcaacgatgaatttcaacgcgtcgccgatgttgtcggcttcgatttcgtattggttgaattcttcgtccataaagacgtacatcgggtcggcaaaatagctgtacgtacagtttttgcgggacaggatgaccacgtcgaatttgtcgtcggctttgtaaatggtttcggaagcagcgccggtcagcaggtttttcagtttcatgctgactttggcggaagagcggccgcctttgatgtattcggttttttgaacgaccataggatcgttgccgaccataaatacattgccggcgcgcagttcttgtgctgttttcat

SEQ ID NO: 267 - DNA, 699 bp, Neisseria gonorrhoeae
gtgcaggcggatttagcctacgccgccgaacgcattacccacgattatccggaaccaaccggtgcaaaaaaagacaaaaaaataagcacggtaagcgattatttcagaaacatccgtacgcattccgtccaccccagggtgtcggtcggctacgatttcggcagctggaggatagcggcagattatgcccgttacagaaagtggaacaacagtaaatattccgtcaacataaaaagggtgaaagaaaacaatggcagcgggaaaaaactgacgcaagacctgaagacggaaaatcaggaaaacggtacgttccacgccgtttcttctctcggcttgtccgccgtttacgatttcgataccggttcccgcttcaaaccctatgcaggcgtgcgcgtcagctacggacacgtcagacacagcatcgattcgaccaaaaaaacaacagatgttattaccgccccccccactacttctgacggagcacctacaacttataatgctaatccacagacgcaaaacccttatcaccaaagcgacagcatccgccgcgtgggcctcggcgtcatcgccggcgtcggtttcgacatcacgcccaacctgaccctggacaccggctaccgctaccacaactggggacgcctggaaaacacccgcttcaaaacccacgaagcctcattgggcatgcgctaccgcttctga

SEQ ID NO: 268 - RNA, 699 nt, Neisseria gonorrhoeae
gugcaggcggauuuagccuacgccgccgaacgcauuacccacgauuauccggaaccaaccggugcaaaaaaagacaaaaaaauaagcacgguaagcgauuauuucagaaacauccguacgcauuccguccaccccagggugucggucggcuacgauuucggcagcuggaggauagcggcagauuaugcccguuacagaaaguggaacaacaguaaauauuccgucaacauaaaaagggugaaagaaaacaauggcagcgggaaaaaacugacgcaagaccugaagacggaaaaucaggaaaacgguacguuccacgccguuucuucucucggcuuguccgccguuuacgauuucgauaccgguucccgcuucaaacccuaugcaggcgugcgcgucagcuacggacacgucagacacagcaucgauucgaccaaaaaaacaacagauguuauuaccgccccccccacuacuucugacggagcaccuacaacuuauaaugcuaauccacagacgcaaaacccuuaucaccaaagcgacagcauccgccgcgugggccucggcgucaucgccggcgucgguuucgacaucacgcccaaccugacccuggacaccggcuaccgcuaccacaacuggggacgccuggaaaacacccgcuucaaaacccacgaagccucauugggcaugcgcuaccgcuucuga

SEQ ID NO: 269 - DNA, 699 bp, Neisseria gonorrhoeae
tcagaagcggtagcgcatgcccaatgaggcttcgtgggttttgaagcgggtgttttccaggcgtccccagttgtggtagcggtagccggtgtccagggtcaggttgggcgtgatgtcgaaaccgacgccggcgatgacgccgaggcccacgcggcggatgctgtcgctttggtgataagggttttgcgtctgtggattagcattataagttgtaggtgctccgtcagaagtagtggggggggcggtaataacatctgttgtttttttggtcgaatcgatgctgtgtctgacgtgtccgtagctgacgcgcacgcctgcatagggtttgaagcgggaaccggtatcgaaatcgtaaacggcggacaagccgagagaagaaacggcgtggaacgtaccgttttcctgattttccgtcttcaggtcttgcgtcagttttttcccgctgccattgttttctttcaccctttttatgttgacggaatatttactgttgttccactttctgtaacgggcataatctgccgctatcctccagctgccgaaatcgtagccgaccgacaccctggggtggacggaatgcgtacggatgtttctgaaataatcgcttaccgtgcttatttttttgtctttttttgcaccggttggttccggataatcgtgggtaatgcgttcggcggcgtaggctaaatccgcctgcac

SEQ ID NO: 270 - DNA, 681 bp, Neisseria gonorrhoeae
gtgcaggcggatctggcttacgcctacgagcacatcacccgcgattatcccgatgcagccggtgcaaaccaaggcaaaaaaataagcacggtaagcgattatttcaaaaacatccgtacgcattccatccaccccagggtgtcggtcggctacgacttcggcggctggaggatagcggcagattatgcccgttacagaaagtggaacgacaataaatattccgtcgacataaaagagttggaaaacaagaatcagaataagagagacctgaagacggaaaatcaggaaaacggcagcttccacgccgtttcttctctcggcttatcagccgtttacgatttcaaactcaacgacaaattcaaaccctatatcggtgcgcgcgtcgcctacggacacgtcagacacagcatcgattcgaccaaaaaaacaacagagtttcttaccgccgccggtcaggatggcggagcgcctacggtttataataacggaagtacgcaagacgcccatcaagaaagcgacagcatccgccgcgtgggcctcggcgtcatcgccggtatcggtttcgacatcacgcccaagctgaccctggacaccggctaccgctaccacaactggggacgcttggaaaacacccgcttcaaaacccacgaagcctcattgggcgtgcgctaccgcttctga

SEQ ID NO: 271 - RNA, 681 nt, Neisseria gonorrhoeae
gugcaggcggaucuggcuuacgccuacgagcacaucacccgcgauuaucccgaugcagccggugcaaaccaaggcaaaaaaauaagcacgguaagcgauuauuucaaaaacauccguacgcauuccauccaccccagggugucggucggcuacgacuucggcggcuggaggauagcggcagauuaugcccguuacagaaaguggaacgacaauaaauauuccgucgacauaaaagaguuggaaaacaagaaucagaauaagagagaccugaagacggaaaaucaggaaaacggcagcuuccacgccguuucuucucucggcuuaucagccguuuacgauuucaaacucaacgacaaauucaaacccuauaucggugcgcgcgucgccuacggacacgucagacacagcaucgauucgaccaaaaaaacaacagaguuucuuaccgccgccggucaggauggcggagcgccuacgguuuauaauaacggaaguacgcaagacgcccaucaagaaagcgacagcauccgccgcgugggccucggcgucaucgccgguaucgguuucgacaucacgcccaagcugacccuggacaccggcuaccgcuaccacaacuggggacgcuuggaaaacacccgcuucaaaacccacgaagccucauugggcgugcgcuaccgcuucuga

SEQ ID NO: 272 - DNA, 681 bp, Neisseria gonorrhoeae
tcagaagcggtagcgcacgcccaatgaggcttcgtgggttttgaagcgggtgttttccaagcgtccccagttgtggtagcggtagccggtgtccagggtcagcttgggcgtgatgtcgaaaccgataccggcgatgacgccgaggcccacgcggcggatgctgtcgctttcttgatgggcgtcttgcgtacttccgttattataaaccgtaggcgctccgccatcctgaccggcggcggtaagaaactctgttgtttttttggtcgaatcgatgctgtgtctgacgtgtccgtaggcgacgcgcgcaccgatatagggtttgaatttgtcgttgagtttgaaatcgtaaacggctgataagccgagagaagaaacggcgtggaagctgccgttttcctgattttccgtcttcaggtctctcttattctgattcttgttttccaactcttttatgtcgacggaatatttattgtcgttccactttctgtaacgggcataatctgccgctatcctccagccgccgaagtcgtagccgaccgacaccctggggtggatggaatgcgtacggatgtttttgaaataatcgcttaccgtgcttatttttttgccttggtttgcaccggctgcatcgggataatcgcgggtgatgtgctcgtaggcgtaagccagatccgcctgcac

SEQ ID NO: 273 - DNA, 690 bp, Neisseria gonorrhoeae
gtgcaggcggatttagcctacgccgccgaacgcattacccacgattatccggaaccaaccggtacaaaaaaagacaaaataagcacggtaagcgattatttcagaaacatccgtacgcattccatccaccccagggtgtcggtcggctacgacttcggcggctggaggatagcggcagattatgcccgttacagaaagtggaacaacagtaaatattccgtcaacacaaaaaaggtgaacgaaaacaagggcgaaaagataaacgtgacgcaatatctgaaggcggaaaatcaggaaaacggtacgttccacgccgtttcttctctcggcttgtccgccgtttacgatttcaaactcaacgacaaattcaaaccctatatcggtgcgcgcgtcgcctacggacacgtcagacacagcatcgattcgaccaaaaaaacaacagagtttcttaccgccgccggtcaggatggcggagcgcctacggtttataataacggaagtacgcaagacgcccatcaagaaagcgacagcatccgccgcgtgggcctcggcgtcatcgccggcgtcggtttcgacatcacgcccaacctgaccttggacgccgggtaccgctaccacaactggggacgcttggaaaacacccgcttcaaaacccacgaagcctcgttgggcatgcgctaccgcttctga

SEQ ID NO: 274 - RNA, 690 nt, Neisseria gonorrhoeae
gugcaggcggauuuagccuacgccgccgaacgcauuacccacgauuauccggaaccaaccgguacaaaaaaagacaaaauaagcacgguaagcgauuauuucagaaacauccguacgcauuccauccaccccagggugucggucggcuacgacuucggcggcuggaggauagcggcagauuaugcccguuacagaaaguggaacaacaguaaauauuccgucaacacaaaaaaggugaacgaaaacaagggcgaaaagauaaacgugacgcaauaucugaaggcggaaaaucaggaaaacgguacguuccacgccguuucuucucucggcuuguccgccguuuacgauuucaaacucaacgacaaauucaaacccuauaucggugcgcgcgucgccuacggacacgucagacacagcaucgauucgaccaaaaaaacaacagaguuucuuaccgccgccggucaggauggcggagcgccuacgguuuauaauaacggaaguacgcaagacgcccaucaagaaagcgacagcauccgccgcgugggccucggcgucaucgccggcgucgguuucgacaucacgcccaaccugaccuuggacgccggguaccgcuaccacaacuggggacgcuuggaaaacacccgcuucaaaacccacgaagccucguugggcaugcgcuaccgcuucuga

SEQ ID NO: 275 - DNA, 690 bp, Neisseria gonorrhoeae
tcagaagcggtagcgcatgcccaacgaggcttcgtgggttttgaagcgggtgttttccaagcgtccccagttgtggtagcggtacccggcgtccaaggtcaggttgggcgtgatgtcgaaaccgacgccggcgatgacgccgaggcccacgcggcggatgctgtcgctttcttgatgggcgtcttgcgtacttccgttattataaaccgtaggcgctccgccatcctgaccggcggcggtaagaaactctgttgtttttttggtcgaatcgatgctgtgtctgacgtgtccgtaggcgacgcgcgcaccgatatagggtttgaatttgtcgttgagtttgaaatcgtaaacggcggacaagccgagagaagaaacggcgtggaacgtaccgttttcctgattttccgccttcagatattgcgtcacgtttatcttttcgcccttgttttcgttcaccttttttgtgttgacggaatatttactgttgttccactttctgtaacgggcataatctgccgctatcctccagccgccgaagtcgtagccgaccgacaccctggggtggatggaatgcgtacggatgtttctgaaataatcgcttaccgtgcttattttgtctttttttgtaccggttggttccggataatcgtgggtaatgcgttcggcggcgtaggctaaatccgcctgcac

SEQ ID NO: 276 - DNA, 819 bp, Neisseria gonorrhoeae
atgaacaccattttcaaaatcagcgcactgaccctttccgccgctttggcactttccgcctgcggcaaaaaagaagccgcccccgcatctgcatccgaacctgccgccgcttctgccgcgcagggcgacacctcttcaatcggcagcacgatgcagcaggcaagctatgcaatgggcgtggacatcggacgctccctgaaacaaatgaaggaacagggcgcggaaatcgatttgaaagtctttaccgatgccatgcaggcagtgtatgacggcaaagaaatcaaaatgaccgaagagcaggcccaggaagtgatgatgaaattcctgcaggagcagcaggctaaagccgtagaaaaacacaaggcggatgcgaaggccaacaaagaaaaaggcgaagccttcctgaaggaaaatgccgccaaagacggcgtgaagaccactgcttccggtctgcagtacaaaatcaccaaacagggtgaaggcaaacagccgacaaaagacgacatcgttaccgtggaatacgaaggccgcctgattgacggtaccgtattcgacagcagcaaagccaacggcggcccggccaccttccctttgagccaagtgattccgggttggaccgaaggcgtacggcttctgaaagaaggcggcgaagccacgttctacatcccgtccaaccttgcctaccgcgaacagggtgcgggcgaaaaaatcggtccgaacgccactttggtatttgacgtgaaactggtcaaaatcggcgcacccgaaaacgcgcccgccaagcagccggatcaagtcgacatcaaaaaagtaaattaa

SEQ ID NO: 277 - RNA, 819 nt, Neisseria gonorrhoeae
augaacaccauuuucaaaaucagcgcacugacccuuuccgccgcuuuggcacuuuccgccugcggcaaaaaagaagccgcccccgcaucugcauccgaaccugccgccgcuucugccgcgcagggcgacaccucuucaaucggcagcacgaugcagcaggcaagcuaugcaaugggcguggacaucggacgcucccugaaacaaaugaaggaacagggcgcggaaaucgauuugaaagucuuuaccgaugccaugcaggcaguguaugacggcaaagaaaucaaaaugaccgaagagcaggcccaggaagugaugaugaaauuccugcaggagcagcaggcuaaagccguagaaaaacacaaggcggaugcgaaggccaacaaagaaaaaggcgaagccuuccugaaggaaaaugccgccaaagacggcgugaagaccacugcuuccggucugcaguacaaaaucaccaaacagggugaaggcaaacagccgacaaaagacgacaucguuaccguggaauacgaaggccgccugauugacgguaccguauucgacagcagcaaagccaacggcggcccggccaccuucccuuugagccaagugauuccggguuggaccgaaggcguacggcuucugaaagaaggcggcgaagccacguucuacaucccguccaaccuugccuaccgcgaacagggugcgggcgaaaaaaucgguccgaacgccacuuugguauuugacgugaaacuggucaaaaucggcgcacccgaaaacgcgcccgccaagcagccggaucaagucgacaucaaaaaaguaaauuaa

SEQ ID NO: 278 - DNA, 819 bp, Neisseria gonorrhoeae
ttaatttacttttttgatgtcgacttgatccggctgcttggcgggcgcgttttcgggtgcgccgattttgaccagtttcacgtcaaataccaaagtggcgttcggaccgattttttcgcccgcaccctgttcgcggtaggcaaggttggacgggatgtagaacgtggcttcgccgccttctttcagaagccgtacgccttcggtccaacccggaatcacttggctcaaagggaaggtggccgggccgccgttggctttgctgctgtcgaatacggtaccgtcaatcaggcggccttcgtattccacggtaacgatgtcgtcttttgtcggctgtttgccttcaccctgtttggtgattttgtactgcagaccggaagcagtggtcttcacgccgtctttggcggcattttccttcaggaaggcttcgcctttttctttgttggccttcgcatccgccttgtgtttttctacggctttagcctgctgctcctgcaggaatttcatcatcacttcctgggcctgctcttcggtcattttgatttctttgccgtcatacactgcctgcatggcatcggtaaagactttcaaatcgatttccgcgccctgttccttcatttgtttcagggagcgtccgatgtccacgcccattgcatagcttgcctgctgcatcgtgctgccgattgaagaggtgtcgccctgcgcggcagaagcggcggcaggttcggatgcagatgcgggggcggcttcttttttgccgcaggcggaaagtgccaaagcggcggaaagggtcagtgcgctgattttgaaaatggtgttcat

SEQ ID NO: 279 - DNA, 705 bp, Neisseria gonorrhoeae
gtgcaggcggatttagcctacgccgccgaacgcattacccacgattatccggaaccaaccggtgcaaaaaaaggcaaaataagcacggtaagcgattatttcagaaacatccgtacgcattccatccaccccagggtgtcggtcggctacgacttcggcggctggaggatagcggcagattatgcccgttacagaaagtggaacgacaataaatattccgtgaacataaaagagttgggaagaaaggatggtacctcttctagcggccgctatcttaacatacaaacccgaaagacggaaaatcaggaaaacggtacgttccacgccgtttcttctctcggcttgtcaaccgtttacgatttcagagccaacgataaattcaaaccctatatcggcgtgcgcgtcgcctacggacacgtcagacatcaggttcattcaatggaaaaagaaaccacgactgttaccacttacccaagcgacggtagtgcgaaaacttctgttccatcagaaatgccccccaaacctgcctatcacgaaaaccgcagcagccgccgcttgggcttcggcgcgatggcgggcgtgggcatagacgtcgcgcccggtctgaccttggacgccggctaccgctaccactattggggacgcctggaaaacacccgcttcaaaacccacgaagcctcattgggcatgcgctaccgcttctga

SEQ ID NO: 280 - RNA, 705 nt, Neisseria gonorrhoeae
gugcaggcggauuuagccuacgccgccgaacgcauuacccacgauuauccggaaccaaccggugcaaaaaaaggcaaaauaagcacgguaagcgauuauuucagaaacauccguacgcauuccauccaccccagggugucggucggcuacgacuucggcggcuggaggauagcggcagauuaugcccguuacagaaaguggaacgacaauaaauauuccgugaacauaaaagaguugggaagaaaggaugguaccucuucuagcggccgcuaucuuaacauacaaacccgaaagacggaaaaucaggaaaacgguacguuccacgccguuucuucucucggcuugucaaccguuuacgauuucagagccaacgauaaauucaaacccuauaucggcgugcgcgucgccuacggacacgucagacaucagguucauucaauggaaaaagaaaccacgacuguuaccacuuacccaagcgacgguagugcgaaaacuucuguuccaucagaaaugccccccaaaccugccuaucacgaaaaccgcagcagccgccgcuugggcuucggcgcgauggcgggcgugggcauagacgucgcgcccggucugaccuuggacgccggcuaccgcuaccacuauuggggacgccuggaaaacacccgcuucaaaacccacgaagccucauugggcaugcgcuaccgcuucuga

SEQ ID NO: 281 - DNA, 705 bp, Neisseria gonorrhoeae
tcagaagcggtagcgcatgcccaatgaggcttcgtgggttttgaagcgggtgttttccaggcgtccccaatagtggtagcggtagccggcgtccaaggtcagaccgggcgcgacgtctatgcccacgcccgccatcgcgccgaagcccaagcggcggctgctgcggttttcgtgataggcaggtttggggggcatttctgatggaacagaagttttcgcactaccgtcgcttgggtaagtggtaacagtcgtggtttctttttccattgaatgaacctgatgtctgacgtgtccgtaggcgacgcgcacgccgatatagggtttgaatttatcgttggctctgaaatcgtaaacggttgacaagccgagagaagaaacggcgtggaacgtaccgttttcctgattttccgtctttcgggtttgtatgttaagatagcggccgctagaagaggtaccatcctttcttcccaactcttttatgttcacggaatatttattgtcgttccactttctgtaacgggcataatctgccgctatcctccagccgccgaagtcgtagccgaccgacaccctggggtggatggaatgcgtacggatgtttctgaaataatcgcttaccgtgcttattttgcctttttttgcaccggttggttccggataatcgtgggtaatgcgttcggcggcgtaggctaaatccgcctgcac

SEQ ID NO: 282 - DNA, 837 bp, Neisseria gonorrhoeae
atgaatccagcccgcaaaaaaccttctcttctcttctcttctcttctcttctcttctcttctcttctcttctcttctcttctcttcggcagcgcaggcggcaagtgaaggcaatggccgcggcccgtatgtgcaggcggatttagcctacgccgccgaacgcattacccacgattatccggaaccaaccgctccaggcaaaaacaaaataagcacggtaagcgattatttcagaaacatccgtacgcattccatccaccccagggtgtcggtcggctacgacttcggcggctggcgcatcgccgcggattatgcccgttacaggaaatggcacaacaataaatattccgtgaacataaaagagttggaaagaaagaataataaaacttttggcggcaaccagcttaacataaaataccaaaagacggaacatcaggaaaacggcacattccacgccgtttcttctctcggcttgtccgccgtttacgatttcaaactcaacgacaaattcaaaccctatatcggtgcgcgcgtcgcctacggacacgtcagacacagcatcgattcgactaaaaaaataacaggtactcttaccgcctaccctagtgatgctgacgcagcagttacggtttatcctgacggacatccgcaaaaaaacacctatcaaaaaagcaacagcagccgccgcttgggcttcggcgcgatggcgggcgtgggcatagacgtcgcgcccggcctgaccttggacgccggctaccgctaccacaactggggacgcttggaaaacacccgcttcaaaacccacgaagcctcgttgggcatgcgctaccgcttctga

SEQ ID NO: 283 - RNA, 837 nt, Neisseria gonorrhoeae
augaauccagcccgcaaaaaaccuucucuucucuucucuucucuucucuucucuucucuucucuucucuucucuucucuucucuucggcagcgcaggcggcaagugaaggcaauggccgcggcccguaugugcaggcggauuuagccuacgccgccgaacgcauuacccacgauuauccggaaccaaccgcuccaggcaaaaacaaaauaagcacgguaagcgauuauuucagaaacauccguacgcauuccauccaccccagggugucggucggcuacgacuucggcggcuggcgcaucgccgcggauuaugcccguuacaggaaauggcacaacaauaaauauuccgugaacauaaaagaguuggaaagaaagaauaauaaaacuuuuggcggcaaccagcuuaacauaaaauaccaaaagacggaacaucaggaaaacggcacauuccacgccguuucuucucucggcuuguccgccguuuacgauuucaaacucaacgacaaauucaaacccuauaucggugcgcgcgucgccuacggacacgucagacacagcaucgauucgacuaaaaaaauaacagguacucuuaccgccuacccuagugaugcugacgcagcaguuacgguuuauccugacggacauccgcaaaaaaacaccuaucaaaaaagcaacagcagccgccgcuugggcuucggcgcgauggcgggcgugggcauagacgucgcgcccggccugaccuuggacgccggcuaccgcuaccacaacuggggacgcuuggaaaacacccgcuucaaaacccacgaagccucguugggcaugcgcuaccgcuucuga

SEQ ID NO: 284 - DNA, 837 bp, Neisseria gonorrhoeae
tcagaagcggtagcgcatgcccaacgaggcttcgtgggttttgaagcgggtgttttccaagcgtccccagttgtggtagcggtagccggcgtccaaggtcaggccgggcgcgacgtctatgcccacgcccgccatcgcgccgaagcccaagcggcggctgctgttgcttttttgataggtgtttttttgcggatgtccgtcaggataaaccgtaactgctgcgtcagcatcactagggtaggcggtaagagtacctgttatttttttagtcgaatcgatgctgtgtctgacgtgtccgtaggcgacgcgcgcaccgatatagggtttgaatttgtcgttgagtttgaaatcgtaaacggcggacaagccgagagaagaaacggcgtggaatgtgccgttttcctgatgttccgtcttttggtattttatgttaagctggttgccgccaaaagttttattattctttctttccaactcttttatgttcacggaatatttattgttgtgccatttcctgtaacgggcataatccgcggcgatgcgccagccgccgaagtcgtagccgaccgacaccctggggtggatggaatgcgtacggatgtttctgaaataatcgcttaccgtgcttattttgtttttgcctggagcggttggttccggataatcgtgggtaatgcgttcggcggcgtaggctaaatccgcctgcacatacgggccgcggccattgccttcacttgccgcctgcgctgccgaagagaagagaagagaagagaagagaagagaagagaagagaagagaagagaagagaagagaaggttttttgcgggctggattcat

SEQ ID NO: 285 - DNA, 699 bp, Neisseria gonorrhoeae; identical to SEQ ID NO: 267

SEQ ID NO: 286 - RNA, 699 nt, Neisseria gonorrhoeae; identical to SEQ ID NO: 268

SEQ ID NO: 287 - DNA, 699 bp, Neisseria gonorrhoeae; identical to SEQ ID NO: 269

SEQ ID NO: 288 - DNA, 237 bp, Neisseria gonorrhoeae
atgtcaaacatcgaacaacaagttaagaaaattattgctgaacaactgggcgtaaacgaagccgacgtgaaaaacgaatcttccttccaagacgacttgggcgcggattctttggataccgtggagttggttatggctttggaagaagccttcggctgcgaaatccccgacgaagatgccgaaaaaatcaccaccgtccaactggctatcgactacatcaatgcccacaacggctaa

SEQ ID NO: 289 - RNA, 237 nt, Neisseria gonorrhoeae
augucaaacaucgaacaacaaguuaagaaaauuauugcugaacaacugggcguaaacgaagccgacgugaaaaacgaaucuuccuuccaagacgacuugggcgcggauucuuuggauaccguggaguugguuauggcuuuggaagaagccuucggcugcgaaauccccgacgaagaugccgaaaaaaucaccaccguccaacuggcuaucgacuacaucaaugcccacaacggcuaa

SEQ ID NO: 290 - DNA, 237 bp, Neisseria gonorrhoeae
ttagccgttgtgggcattgatgtagtcgatagccagttggacggtggtgattttttcggcatcttcgtcggggatttcgcagccgaaggcttcttccaaagccataaccaactccacggtatccaaagaatccgcgcccaagtcgtcttggaaggaagattcgtttttcacgtcggcttcgtttacgcccagttgttcagcaataattttcttaacttgttgttcgatgtttgacat

SEQ ID NO: 291 - DNA, 1185 bp, Neisseria gonorrhoeae
atggctaaggaaaaattcgaacgtagcaaaccgcacgtaaacgttggcaccatcggtcacgttgaccatggtaaaaccaccctgactgctgctttgactactattttagctaaaaaattcggcggcgctgcaaaagcttacgaccaaatcgacaacgcacccgaagaaaaagcacgcggtattaccattaacacctcgcacgtagaatacgaaaccgaaacccgccactacgcacacgtagactgtccgggtcacgccgactacgttaaaaacatgattaccggcgccgcacaaatggacggtgcaatcctggtatgttctgctgccgacggccctatgccgcaaacccgcgaacacatcctgctggcccgtcaagtaggcgtaccttacatcatcgtgttcatgaacaaatgcgacatggtcgacgatgccgagctgttggaactggttgaaatggaaatccgcgacctgctgtccagctacgacttccccggcgacgactgcccgatcgtacaaggttccgcactgaaagccttggaaggcgatgccgcttacgaagaaaaaatcttcgaactggctaccgcattggacagctacatcccgactcccgagcgtgccgtggacaaaccattcctgctgcctatcgaagacgtgttctccatttccggccgcggtaccgtagtcaccggccgtgtagagcgaggtatcatccacgttggtgacgagattgaaatcgtcggtctgaaagaaacccaaaaaaccacctgtaccggcgttgaaatgttccgcaaactgctggacgaaggtcaggcgggcgacaacgtaggcgtattgctgcgcggtaccaaacgtgaagacgtagaacgcggtcaggtattggccaaaccgggtactatcactcctcacaccaagttcaaagcagaagtgtacgtattgagcaaagaagagggcggccgccataccccgtttttcgccaactaccgtccccaattctacttccgtaccactgacgtaaccggcgcggttactttggaaaaaggtgtggaaatggtaatgccgggtgagaacgtaaccattactgtagaactgattgcgcctatcgctatggaagaaggtctgcgctttgcgattcgcgaaggcggccgtaccgtgggtgccggcgtggtttcttctgttatcgcttaa

SEQ ID NO: 292 - RNA, 1185 nt, Neisseria gonorrhoeae
auggcuaaggaaaaauucgaacguagcaaaccgcacguaaacguuggcaccaucggucacguugaccaugguaaaaccacccugacugcugcuuugacuacuauuuuagcuaaaaaauucggcggcgcugcaaaagcuuacgaccaaaucgacaacgcacccgaagaaaaagcacgcgguauuaccauuaacaccucgcacguagaauacgaaaccgaaacccgccacuacgcacacguagacuguccgggucacgccgacuacguuaaaaacaugauuaccggcgccgcacaaauggacggugcaauccugguauguucugcugccgacggcccuaugccgcaaacccgcgaacacauccugcuggcccgucaaguaggcguaccuuacaucaucguguucaugaacaaaugcgacauggucgacgaugccgagcuguuggaacugguugaaauggaaauccgcgaccugcuguccagcuacgacuuccccggcgacgacugcccgaucguacaagguuccgcacugaaagccuuggaaggcgaugccgcuuacgaagaaaaaaucuucgaacuggcuaccgcauuggacagcuacaucccgacucccgagcgugccguggacaaaccauuccugcugccuaucgaagacguguucuccauuuccggccgcgguaccguagucaccggccguguagagcgagguaucauccacguuggugacgagauugaaaucgucggucugaaagaaacccaaaaaaccaccuguaccggcguugaaauguuccgcaaacugcuggacgaaggucaggcgggcgacaacguaggcguauugcugcgcgguaccaaacgugaagacguagaacgcggucagguauuggccaaaccggguacuaucacuccucacaccaaguucaaagcagaaguguacguauugagcaaagaagagggcggccgccauaccccguuuuucgccaacuaccguccccaauucuacuuccguaccacugacguaaccggcgcgguuacuuuggaaaaagguguggaaaugguaaugccgggugagaacguaaccauuacuguagaacugauugcgccuaucgcuauggaagaaggucugcgcuuugcgauucgcgaaggcggccguaccgugggugccggcgugguuucuucuguuaucgcuuaa

SEQ ID NO: 293 - DNA, 1185 bp, Neisseria gonorrhoeae
ttaagcgataacagaagaaaccacgccggcacccacggtacggccgccttcgcgaatcgcaaagcgcagaccttcttccatagcgataggcgcaatcagttctacagtaatggttacgttctcacccggcattaccatttccacacctttttccaaagtaaccgcgccggttacgtcagtggtacggaagtagaattggggacggtagttggcgaaaaacggggtatggcggccgccctcttctttgctcaatacgtacacttctgctttgaacttggtgtgaggagtgatagtacccggtttggccaatacctgaccgcgttctacgtcttcacgtttggtaccgcgcagcaatacgcctacgttgtcgcccgcctgaccttcgtccagcagtttgcggaacatttcaacgccggtacaggtggttttttgggtttctttcagaccgacgatttcaatctcgtcaccaacgtggatgatacctcgctctacacggccggtgactacggtaccgcggccggaaatggagaacacgtcttcgataggcagcaggaatggtttgtccacggcacgctcgggagtcgggatgtagctgtccaatgcggtagccagttcgaagattttttcttcgtaagcggcatcgccttccaaggctttcagtgcggaaccttgtacgatcgggcagtcgtcgccggggaagtcgtagctggacagcaggtcgcggatttccatttcaaccagttccaacagctcggcatcgtcgaccatgtcgcatttgttcatgaacacgatgatgtaaggtacgcctacttgacgggccagcaggatgtgttcgcgggtttgcggcatagggccgtcggcagcagaacataccaggattgcaccgtccatttgtgcggcgccggtaatcatgtttttaacgtagtcggcgtgacccggacagtctacgtgtgcgtagtggcgggtttcggtttcgtattctacgtgcgaggtgttaatggtaataccgcgtgctttttcttcgggtgcgttgtcgatttggtcgtaagcttttgcagcgccgccgaattttttagctaaaatagtagtcaaagcagcagtcagggtggttttaccatggtcaacgtgaccgatggtgccaacgtttacgtgcggtttgctacgttcgaatttttccttagccat

SEQ ID NO: 294 - DNA, 504 bp, Neisseria gonorrhoeae
atggctttactgaatatcttgcaatatcccgacgagcgtctgcacacggtggcaaagcctgtcgaacaagttgacgagcgcatccggaagctggttgccgatatgtttgaaacgatgtacgaatcgcgcggcatcgggctggcggcgacgcaggtcgatgtgcacgaacgcgtggtcgtgatggatttgaccgaagaccgcagcgaaccgcgcgtgttcatcaaccccgtcatcgttgaaaaagacggcgaaaccacttacgaagagggctgcctgtccgtaccgggcatttacgacgccgttacccgcgccgaacgcgtcaaggtcgaggctttgaacgaaaaaggcgaaaaattcacgctggaggcggacgggctgctggcgatttgcgtgcagcacgagttagatcacctgatgggcatcgtgtttgtcgaacgcctttcccaactcaagcaggggcggattaagaccaaactgaaaaaacgtcagaaacatacgatttga

SEQ ID NO: 295 - RNA, 504 nt, Neisseria gonorrhoeae
auggcuuuacugaauaucuugcaauaucccgacgagcgucugcacacgguggcaaagccugucgaacaaguugacgagcgcauccggaagcugguugccgauauguuugaaacgauguacgaaucgcgcggcaucgggcuggcggcgacgcaggucgaugugcacgaacgcguggucgugauggauuugaccgaagaccgcagcgaaccgcgcguguucaucaaccccgucaucguugaaaaagacggcgaaaccacuuacgaagagggcugccuguccguaccgggcauuuacgacgccguuacccgcgccgaacgcgucaaggucgaggcuuugaacgaaaaaggcgaaaaauucacgcuggaggcggacgggcugcuggcgauuugcgugcagcacgaguuagaucaccugaugggcaucguguuugucgaacgccuuucccaacucaagcaggggcggauuaagaccaaacugaaaaaacgucagaaacauacgauuuga

SEQ ID NO: 296 - DNA, 504 bp, Neisseria gonorrhoeae
tcaaatcgtatgtttctgacgttttttcagtttggtcttaatccgcccctgcttgagttgggaaaggcgttcgacaaacacgatgcccatcaggtgatctaactcgtgctgcacgcaaatcgccagcagcccgtccgcctccagcgtgaatttttcgcctttttcgttcaaagcctcgaccttgacgcgttcggcgcgggtaacggcgtcgtaaatgcccggtacggacaggcagccctcttcgtaagtggtttcgccgtctttttcaacgatgacggggttgatgaacacgcgcggttcgctgcggtcttcggtcaaatccatcacgaccacgcgttcgtgcacatcgacctgcgtcgccgccagcccgatgccgcgcgattcgtacatcgtttcaaacatatcggcaaccagcttccggatgcgctcgtcaacttgttcgacaggctttgccaccgtgtgcagacgctcgtcgggatattgcaagatattcagtaaagccat

SEQ ID NO: 297 - DNA, 1044 bp, Neisseria gonorrhoeae
atgcagattaccgacttactcgccttcggcgctaaaaacaaagcatccgaccttcacctgagttcgggcatatcccctatgattcgggttcacggcgacatgcggcgcatcaaccttcccgaaatgagcgcggaagaggtcggcaatatggtaacttcggtgatgaacgaccaccagcggaaaatctaccagcaaaacttggaagtcgacttctcgttcgaactgcccaacgtcgcccgattccgcgtcaacgccttcaacaccggccgcggccccgccgccgtattccgcaccattcccagcaccgtcttatcgctggaagaattgaaagccccgagcattttccaaaaaatcgcagaatcgccgcgcggcatggtattggttaccggccctaccggttcgggcaaatcgaccacgcttgccgcgatgatcaactacatcaacgaaacccagccggcacacatcctgaccatcgaagacccgatcgaattcgtccaccaaagcaaaaaatccctgattaaccaacgcgagctgcaccagcacaccctcagcttcgccaacgcgctgagttccgcattgcgcgaagaccccgacgttatccttgtcggcgagatgcgcgaccccgaaaccatcggcttggcactgaccgccgccgaaaccggacacttggttttcggcacgctgcacacgaccggcgcggcaaaaaccgtcgaccgtatcgtggacgtattccccgccggagagaaagaaatggtgcgttccatgctgtccgaatcgctgaccgccgtcatctcccaaaacctgctgaaaacgcacgacggcgacggccgtgtcgcctcgcacgaaatcctgattgccaaccccgccgtccgcaacctcatccgcgaaaacaaaatcacgcagattaactccgtcctgcaaaccgggcgggcgagcggtatgcagacgatggaccaatcgctgcaatcgctggtgcgccaagggctgatcgcaccggaagccacacgcagacgcgcgcaaaacagcgaaagtatgagtttctga

SEQ ID NO: 298 - RNA, 1044 nt, Neisseria gonorrhoeae
augcagauuaccgacuuacucgccuucggcgcuaaaaacaaagcauccgaccuucaccugaguucgggcauauccccuaugauucggguucacggcgacaugcggcgcaucaaccuucccgaaaugagcgcggaagaggucggcaauaugguaacuucggugaugaacgaccaccagcggaaaaucuaccagcaaaacuuggaagucgacuucucguucgaacugcccaacgucgcccgauuccgcgucaacgccuucaacaccggccgcggccccgccgccguauuccgcaccauucccagcaccgucuuaucgcuggaagaauugaaagccccgagcauuuuccaaaaaaucgcagaaucgccgcgcggcaugguauugguuaccggcccuaccgguucgggcaaaucgaccacgcuugccgcgaugaucaacuacaucaacgaaacccagccggcacacauccugaccaucgaagacccgaucgaauucguccaccaaagcaaaaaaucccugauuaaccaacgcgagcugcaccagcacacccucagcuucgccaacgcgcugaguuccgcauugcgcgaagaccccgacguuauccuugucggcgagaugcgcgaccccgaaaccaucggcuuggcacugaccgccgccgaaaccggacacuugguuuucggcacgcugcacacgaccggcgcggcaaaaaccgucgaccguaucguggacguauuccccgccggagagaaagaaauggugcguuccaugcuguccgaaucgcugaccgccgucaucucccaaaaccugcugaaaacgcacgacggcgacggccgugucgccucgcacgaaauccugauugccaaccccgccguccgcaaccucauccgcgaaaacaaaaucacgcagauuaacuccguccugcaaaccgggcgggcgagcgguaugcagacgauggaccaaucgcugcaaucgcuggugcgccaagggcugaucgcaccggaagccacacgcagacgcgcgcaaaacagcgaaaguaugaguuucuga

SEQ ID NO: 299 - DNA, 1044 bp, Neisseria gonorrhoeae
tcagaaactcatactttcgctgttttgcgcgcgtctgcgtgtggcttccggtgcgatcagcccttggcgcaccagcgattgcagcgattggtccatcgtctgcataccgctcgcccgcccggtttgcaggacggagttaatctgcgtgattttgttttcgcggatgaggttgcggacggcggggttggcaatcaggatttcgtgcgaggcgacacggccgtcgccgtcgtgcgttttcagcaggttttgggagatgacggcggtcagcgattcggacagcatggaacgcaccatttctttctctccggcggggaatacgtccacgatacggtcgacggtttttgccgcgccggtcgtgtgcagcgtgccgaaaaccaagtgtccggtttcggcggcggtcagtgccaagccgatggtttcggggtcgcgcatctcgccgacaaggataacgtcggggtcttcgcgcaatgcggaactcagcgcgttggcgaagctgagggtgtgctggtgcagctcgcgttggttaatcagggattttttgctttggtggacgaattcgatcgggtcttcgatggtcaggatgtgtgccggctgggtttcgttgatgtagttgatcatcgcggcaagcgtggtcgatttgcccgaaccggtagggccggtaaccaataccatgccgcgcggcgattctgcgattttttggaaaatgctcggggctttcaattcttccagcgataagacggtgctgggaatggtgcggaatacggcggcggggccgcggccggtgttgaaggcgttgacgcggaatcgggcgacgttgggcagttcgaacgagaagtcgacttccaagttttgctggtagattttccgctggtggtcgttcatcaccgaagttaccatattgccgacctcttccgcgctcatttcgggaaggttgatgcgccgcatgtcgccgtgaacccgaatcataggggatatgcccgaactcaggtgaaggtcggatgctttgtttttagcgccgaaggcgagtaagtcggtaatctgcat

SEQ ID NO: 300 - DNA, 300 bp, Neisseria gonorrhoeae
atgaaaatatttgaaaatatagaagatgttaaagccatccgtaaaaagaccgggatgaaccagatagacttctggggcaaggtcggcgttactcaatccggaggttcacgctacgaaaccggccgtaagatgcccaaacccgtacgcgaactgctccgcctcgtccatatcgaatgcctcgatttggcaaaagtcaacaaaaaagatatggaaatcgccgccctgttgaaaaaacaccatcccgacctgtatgccgagttgtccaaacagaccaagtccgaaagaaaaaaacaaagttaa

SEQ ID NO: 301 - RNA, 300 nt, Neisseria gonorrhoeae
augaaaauauuugaaaauauagaagauguuaaagccauccguaaaaagaccgggaugaaccagauagacuucuggggcaaggucggcguuacucaauccggagguucacgcuacgaaaccggccguaagaugcccaaacccguacgcgaacugcuccgccucguccauaucgaaugccucgauuuggcaaaagucaacaaaaaagauauggaaaucgccgcccuguugaaaaaacaccaucccgaccuguaugccgaguuguccaaacagaccaaguccgaaagaaaaaaacaaaguuaa

SEQ ID NO: 302 - DNA, 300 bp, Neisseria gonorrhoeae
ttaactttgtttttttctttcggacttggtctgtttggacaactcggcatacaggtcgggatggtgttttttcaacagggcggcgatttccatatcttttttgttgacttttgccaaatcgaggcattcgatatggacgaggcggagcagttcgcgtacgggtttgggcatcttacggccggtttcgtagcgtgaacctccggattgagtaacgccgaccttgccccagaagtctatctggttcatcccggtctttttacggatggctttaacatcttctatattttcaaatattttcat

SEQ ID NO: 303 - DNA, 684 bp, Neisseria gonorrhoeae
gtgcaggcggatttagcctacgccgccgaacgcattacccacgattatccggaaccaaccgctccaggcaaaaacaaaataagcacggtaagcgattatttcagaaacatccgtacgcattccatccaccccagggtgtcggtcggctacgacttcggcggctggaggatagcggcagattatgcccgttacagaaagtggaacgacaataaatattccgtcgacataaaagagttggaaaacaagaatcagaataagagagacctgaagacggaaaatcaggaaaacggcagcttccacgccgtttcttctctcggcttatcagccgtttacgatttcaaactcaacgacaaattcaaaccctatatcggtgcgcgcgtcgcctacggacacgtcagacacagcatcgattcgactaaaaaaataacaggtactcttaccgcctaccctagtgatgctgacgcagcagttacggtttatcctgacggacatccgcaaaaaaacacctatcaaaaaagcaacagcagccgccgcttgggcttcggcgcgatggcgggcgtgggcatagacgtcgcgcccggcctgaccttggacgccggctaccgctaccacaactggggacgcttggaaaacacccgcttcaaaacccacgaagcctcattgggcatgcgctaccgcttctga

SEQ ID NO: 304 - RNA, 684 nt, Neisseria gonorrhoeae
gugcaggcggauuuagccuacgccgccgaacgcauuacccacgauuauccggaaccaaccgcuccaggcaaaaacaaaauaagcacgguaagcgauuauuucagaaacauccguacgcauuccauccaccccagggugucggucggcuacgacuucggcggcuggaggauagcggcagauuaugcccguuacagaaaguggaacgacaauaaauauuccgucgacauaaaagaguuggaaaacaagaaucagaauaagagagaccugaagacggaaaaucaggaaaacggcagcuuccacgccguuucuucucucggcuuaucagccguuuacgauuucaaacucaacgacaaauucaaacccuauaucggugcgcgcgucgccuacggacacgucagacacagcaucgauucgacuaaaaaaauaacagguacucuuaccgccuacccuagugaugcugacgcagcaguuacgguuuauccugacggacauccgcaaaaaaacaccuaucaaaaaagcaacagcagccgccgcuugggcuucggcgcgauggcgggcgugggcauagacgucgcgcccggccugaccuuggacgccggcuaccgcuaccacaacuggggacgcuuggaaaacacccgcuucaaaacccacgaagccucauugggcaugcgcuaccgcuucuga

SEQ ID NO: 305 - DNA, 684 bp, Neisseria gonorrhoeae
tcagaagcggtagcgcatgcccaatgaggcttcgtgggttttgaagcgggtgttttccaagcgtccccagttgtggtagcggtagccggcgtccaaggtcaggccgggcgcgacgtctatgcccacgcccgccatcgcgccgaagcccaagcggcggctgctgttgcttttttgataggtgtttttttgcggatgtccgtcaggataaaccgtaactgctgcgtcagcatcactagggtaggcggtaagagtacctgttatttttttagtcgaatcgatgctgtgtctgacgtgtccgtaggcgacgcgcgcaccgatatagggtttgaatttgtcgttgagtttgaaatcgtaaacggctgataagccgagagaagaaacggcgtggaagctgccgttttcctgattttccgtcttcaggtctctcttattctgattcttgttttccaactcttttatgtcgacggaatatttattgtcgttccactttctgtaacgggcataatctgccgctatcctccagccgccgaagtcgtagccgaccgacaccctggggtggatggaatgcgtacggatgtttctgaaataatcgcttaccgtgcttattttgtttttgcctggagcggttggttccggataatcgtgggtaatgcgttcggcggcgtaggctaaatccgcctgcac

SEQ ID NO: 306 - DNA, 684 bp, Neisseria gonorrhoeae
atgcaacacgacgtttacgactacaccgcgcatacggtttctaaaaacaccgtcctgcagaaaacctaccgcctgctcggattttcattcattccggcagccgcaggcgcggcacttgccgccaatgccggtttcaatttttacgccgccttcggttcgcgctggataggatttgccgtcgtattggcgtttttctacggtatgatccacttcatcgaaaaaaaccgttacagcaataccggcgttaccctgctgatggtattcacattcggtatgggcgtattgatcggccccgtgctgcaatacgcactccatattgccgacggtgcgaaaatcgtcggcattgccgccgcgatgaccgccgccgtctttttaacgatgtccgcattggcacgccgaacccggctcgatatgaacgcgctcggacgcttcctgaccgtaggtgcggtcattctgatggtcgccgtggttgccaatctgtttttgggtattcccgcactcgccctgaccatttccgccggttttgtcttgttcagttccttaataattatgtggcaggtacgcaccgtcatcgacggcggcgaagacagttacatcagcgcggcactgacactgtttatctcgctttacaacatcttcagcagcctgctcaacatcctgctgtccttaaacggcgacgactga

SEQ ID NO: 307 - RNA, 684 nt, Neisseria gonorrhoeae
augcaacacgacguuuacgacuacaccgcgcauacgguuucuaaaaacaccguccugcagaaaaccuaccgccugcucggauuuucauucauuccggcagccgcaggcgcggcacuugccgccaaugccgguuucaauuuuuacgccgccuucgguucgcgcuggauaggauuugccgucguauuggcguuuuucuacgguaugauccacuucaucgaaaaaaaccguuacagcaauaccggcguuacccugcugaugguauucacauucgguaugggcguauugaucggccccgugcugcaauacgcacuccauauugccgacggugcgaaaaucgucggcauugccgccgcgaugaccgccgccgucuuuuuaacgauguccgcauuggcacgccgaacccggcucgauaugaacgcgcucggacgcuuccugaccguaggugcggucauucugauggucgccgugguugccaaucuguuuuuggguauucccgcacucgcccugaccauuuccgccgguuuugucuuguucaguuccuuaauaauuauguggcagguacgcaccgucaucgacggcggcgaagacaguuacaucagcgcggcacugacacuguuuaucucgcuuuacaacaucuucagcagccugcucaacauccugcuguccuuaaacggcgacgacuga

SEQ ID NO: 308 - DNA, 684 bp, Neisseria gonorrhoeae
tcagtcgtcgccgtttaaggacagcaggatgttgagcaggctgctgaagatgttgtaaagcgagataaacagtgtcagtgccgcgctgatgtaactgtcttcgccgccgtcgatgacggtgcgtacctgccacataattattaaggaactgaacaagacaaaaccggcggaaatggtcagggcgagtgcgggaatacccaaaaacagattggcaaccacggcgaccatcagaatgaccgcacctacggtcaggaagcgtccgagcgcgttcatatcgagccgggttcggcgtgccaatgcggacatcgttaaaaagacggcggcggtcatcgcggcggcaatgccgacgattttcgcaccgtcggcaatatggagtgcgtattgcagcacggggccgatcaatacgcccataccgaatgtgaataccatcagcagggtaacgccggtattgctgtaacggtttttttcgatgaagtggatcataccgtagaaaaacgccaatacgacggcaaatcctatccagcgcgaaccgaaggcggcgtaaaaattgaaaccggcattggcggcaagtgccgcgcctgcggctgccggaatgaatgaaaatccgagcaggcggtaggttttctgcaggacggtgtttttagaaaccgtatgcgcggtgtagtcgtaaacgtcgtgttgcat

SEQ ID NO: 309 - DNA, 213 bp, Neisseria gonorrhoeae
atgcctgcaatccgcgtaaaagagaatgaaccatttgaagtcgctatgcgccgtttcaaacgcgccgtagaaaaaaccggcctgctgaccgagctgcgcgcccgcgaagcctacgaaaaaccgactaccgaacgcaaacgcaaaaaagcggcagccgtaaaacgcctgcaaaaacgcctgcgcagccaacagctgccgcccaaaatgtactaa

SEQ ID NO: 310 - RNA, 213 nt, Neisseria gonorrhoeae
augccugcaauccgcguaaaagagaaugaaccauuugaagucgcuaugcgccguuucaaacgcgccguagaaaaaaccggccugcugaccgagcugcgcgcccgcgaagccuacgaaaaaccgacuaccgaacgcaaacgcaaaaaagcggcagccguaaaacgccugcaaaaacgccugcgcagccaacagcugccgcccaaaauguacuaa

SEQ ID NO: 311 - DNA, 213 bp, Neisseria gonorrhoeae
ttagtacattttgggcggcagctgttggctgcgcaggcgtttttgcaggcgttttacggctgccgcttttttgcgtttgcgttcggtagtcggtttttcgtaggcttcgcgggcgcgcagctcggtcagcaggccggttttttctacggcgcgtttgaaacggcgcatagcgacttcaaatggttcattctcttttacgcggattgcaggcat

SEQ ID NO: 312 - DNA, 237 bp, Neisseria gonorrhoeae
atgggtttgattgctatcgcatgtggtttgatcgttgcattgggtgcattgggtgcatctatcggtatcgcaatggtcggttctaaatatttggagtcttctgctcgccaacctgaactgattggtccgctgcaaaccaaactgttcctgattgccggtctgattgatgccgcattcttgatcggtgtcgccattgcactactgttcgccttcgtcaacccgtttgcaggtgcataa

SEQ ID NO: 313 - RNA, 237 nt, Neisseria gonorrhoeae
auggguuugauugcuaucgcaugugguuugaucguugcauugggugcauugggugcaucuaucgguaucgcaauggucgguucuaaauauuuggagucuucugcucgccaaccugaacugauugguccgcugcaaaccaaacuguuccugauugccggucugauugaugccgcauucuugaucggugucgccauugcacuacuguucgccuucgucaacccguuugcaggugcauaa

SEQ ID NO: 314 - DNA, 237 bp, Neisseria gonorrhoeae
ttatgcacctgcaaacgggttgacgaaggcgaacagtagtgcaatggcgacaccgatcaagaatgcggcatcaatcagaccggcaatcaggaacagtttggtttgcagcggaccaatcagttcaggttggcgagcagaagactccaaatatttagaaccgaccattgcgataccgatagatgcacccaatgcacccaatgcaacgatcaaaccacatgcgatagcaatcaaacccat

SEQ ID NO: 315 - DNA, 471 bp, Neisseria gonorrhoeae
gtgaatatcaatgcaacattattcgctcaaatcatcgtctttttcggtttggtatggtttaccatgaaatttgtgtggccgccgattgcaaaagctttggatgagcgtgccgcaaaaatcgccgagggcttggctgccgccgagcgtggtaaaagcgatttcgagcaggctgaaaaaaaggttgcagaacttttggcagaagggcgtaatcaggtttccgaaatggttgccaacgccgaaaaacgtgccgccaaaattgtcgaagaagccaaagaacaggcttcttccgaggcggcgcgcattgcagctcaggcaaaggccgatgtggagcaggaattgttccgcgcacgcgaatccctgcgcgatcaggttgccgtgttggctgtcaaaggtgccgaatctattttgcgcagcgaagtcgatgcttccaaacacgcaaaactgctcgataccctgaaacaggagttgtaa

SEQ ID NO: 316 - RNA, 471 nt, Neisseria gonorrhoeae
gugaauaucaaugcaacauuauucgcucaaaucaucgucuuuuucgguuugguaugguuuaccaugaaauuuguguggccgccgauugcaaaagcuuuggaugagcgugccgcaaaaaucgccgagggcuuggcugccgccgagcgugguaaaagcgauuucgagcaggcugaaaaaaagguugcagaacuuuuggcagaagggcguaaucagguuuccgaaaugguugccaacgccgaaaaacgugccgccaaaauugucgaagaagccaaagaacaggcuucuuccgaggcggcgcgcauugcagcucaggcaaaggccgauguggagcaggaauuguuccgcgcacgcgaaucccugcgcgaucagguugccguguuggcugucaaaggugccgaaucuauuuugcgcagcgaagucgaugcuuccaaacacgcaaaacugcucgauacccugaaacaggaguuguaa

SEQ ID NO: 317 - DNA, 471 bp, Neisseria gonorrhoeae
ttacaactcctgtttcagggtatcgagcagttttgcgtgtttggaagcatcgacttcgctgcgcaaaatagattcggcacctttgacagccaacacggcaacctgatcgcgcagggattcgcgtgcgcggaacaattcctgctccacatcggcctttgcctgagctgcaatgcgcgccgcctcggaagaagcctgttctttggcttcttcgacaattttggcggcacgtttttcggcgttggcaaccatttcggaaacctgattacgcccttctgccaaaagttctgcaacctttttttcagcctgctcgaaatcgcttttaccacgctcggcggcagccaagccctcggcgatttttgcggcacgctcatccaaagcttttgcaatcggcggccacacaaatttcatggtaaaccataccaaaccgaaaaagacgatgatttgagcgaataatgttgcattgatattcac

SEQ ID NO: 318 - DNA, 73 bp, Neisseria gonorrhoeae
aggccaatagctcaattggtagagtatcggtctccaaaaccgagggttgggggttcgagaccctcttggcctg

SEQ ID NO: 319 - RNA, 73 nt, Neisseria gonorrhoeae
aggccaauagcucaauugguagaguaucggucuccaaaaccgaggguuggggguucgagacccucuuggccug

SEQ ID NO: 320 - DNA, 73 bp, Neisseria gonorrhoeae
caggccaagagggtctcgaacccccaaccctcggttttggagaccgatactctaccaattgagctattggcct

SEQ ID NO: 321 - DNA, 71 bp, Neisseria gonorrhoeae
gcgggtgtagctcaatggtagagcagaagccttccaagcttacggtgagggttcgattcccttcacccgct

SEQ ID NO: 322 - RNA, 71 nt, Neisseria gonorrhoeae
gcggguguagcucaaugguagagcagaagccuuccaagcuuacggugaggguucgauucccuucacccgcu

SEQ ID NO: 323 - DNA, 71 bp, Neisseria gonorrhoeae
agcgggtgaagggaatcgaaccctcaccgtaagcttggaaggcttctgctctaccattgagctacacccgc

SEQ ID NO: 324 - DNA, 2910 bp, Neisseria gonorrhoeae; identical to SEQ ID NO: 1

SEQ ID NO: 325 - RNA, 2910 nt, Neisseria gonorrhoeae; identical to SEQ ID NO: 3

SEQ ID NO: 326 - DNA, 2910 bp, Neisseria gonorrhoeae; identical to SEQ ID NO: 2

SEQ ID NO: 327 - DNA, 1545 bp, Neisseria gonorrhoeae; identical to SEQ ID NO: 4

SEQ ID NO: 328 - RNA, 1545 nt, Neisseria gonorrhoeae; identical to SEQ ID NO: 6

SEQ ID NO: 329 - DNA, 1545 bp, Neisseria gonorrhoeae; identical to SEQ ID NO: 5

SEQ ID NO: 330 - DNA, 2904 bp, Neisseria gonorrhoeae; identical to SEQ ID NO: 7

SEQ ID NO: 331 - RNA, 2904 nt, Neisseria gonorrhoeae; identical to SEQ ID NO: 9

SEQ ID NO: 332 - DNA, 2904 bp, Neisseria gonorrhoeae; identical to SEQ ID NO: 8

SEQ ID NO: 333 - DNA, 1545 bp, Neisseria gonorrhoeae; identical to SEQ ID NO: 4

SEQ ID NO: 334 - RNA, 1545 nt, Neisseria gonorrhoeae; identical to SEQ ID NO: 6

SEQ ID NO: 335 - DNA, 1545 bp, Neisseria gonorrhoeae; identical to SEQ ID NO: 5

SEQ ID NO: 336 - DNA, 2903 bp, Neisseria gonorrhoeae; identical to SEQ ID NO: 16

SEQ ID NO: 337 - RNA, 2903 nt, Neisseria gonorrhoeae; identical to SEQ ID NO: 18

SEQ ID NO: 338 - DNA, 2903 bp, Neisseria gonorrhoeae; identical to SEQ ID NO: 17

SEQ ID NO: 339 - DNA, 1545 bp, Neisseria gonorrhoeae; identical to SEQ ID NO: 4

SEQ ID NO: 340 - RNA, 1545 nt, Neisseria gonorrhoeae; identical to SEQ ID NO: 6

SEQ ID NO: 341 - DNA, 1545 bp, Neisseria gonorrhoeae; identical to SEQ ID NO: 5

SEQ ID NO: 342 - DNA, 2903 bp, Neisseria gonorrhoeae; identical to SEQ ID NO: 22

SEQ ID NO: 343 - RNA, 2903 nt, Neisseria gonorrhoeae; identical to SEQ ID NO: 24

SEQ ID NO: 344 - DNA, 2903 bp, Neisseria gonorrhoeae; identical to SEQ ID NO: 23

(19) United States
(12) Patent Application Publication
(10) Pub. No.: US 2023/0183818 A1
(43) Pub. Date: Jun. 15, 2023

(54) ANTIBIOTIC SUSCEPTIBILITY OF MICROORGANISMS AND RELATED MARKERS, COMPOSITIONS, METHODS AND SYSTEMS

(71) Applicant: California Institute of Technology, Pasadena, CA (US)

(72) Inventors: Rustem F. ISMAGILOV, Pasadena, CA (US); Nathan SCHOEPP, Pasadena, CA (US); Tahmineh KHAZAEI, Pasadena, CA (US); Jacob T. BARLOW, Camperdown (AU)

(21) Appl. No.: 17/264,734

(22) PCT Filed: Aug. 1, 2019
(86) PCT No.: PCT/US2019/044748

§ 371 Date: Jan. 29, 2021

Related U.S. Application Data
(60) Provisional application No. 62/713,412, filed on Aug. 1, 2018.

(51) Int. Cl. C12Q 1/689; C12Q 1/02; C12Q 1/04

(52) U.S. Cl. CPC C12Q 1/689; C12Q 1/04; C12Q 1/025; C12Q 2600/106; C12Q 2600/158

(57) ABSTRACT

Provided herein are RNA markers and compositions, methods and systems for the related identification and/or uses in methods for detection of antibiotic susceptibility and resistance in a microorganism, and in particular in N. gonorrhoeae.

### CROSS REFERENCE TO RELATED APPLICATIONS

The present application is the U.S. National Stage of International Patent Application No. PCT/US2019/044748, entitled “Antibiotic Susceptibility of Microorganisms and Related Markers, Compositions, methods and Systems,” filed on Aug. 1, 2019 which claims priority to U.S. Provisional Application No. 62/713,412, entitled “Antibiotic Susceptibility of Microorganisms and Related Markers, Compositions, methods and Systems” filed on Aug. 1, 2018 with docket number P2255-USP, the content of which is incorporated herein by reference in its entirety.

### STATEMENT OF GOVERNMENT GRANT

This invention was made with government support under Federal Award No. IDSEP160030-02awarded by the Department of Health and Human Services (HHS) Office of the Assistant Secretary for Preparedness and Response (ASPR) and the Wellcome Trust under the CARB-X program. The government has certain rights in the invention.

### FIELD

The present disclosure relates to microorganisms and related biology as well as to diagnosis and treatment of related conditions in individuals. In particular, the present disclosure relates to antibiotic susceptibility of microorganisms and related markers, compositions, methods and systems.

### BACKGROUND

Antibiotic susceptibility is an important feature of the biology of various microorganisms, which can be used in identifying approaches to treat or prevent bacterial infections.

Ideal antibiotic therapy is based on determination of the etiological agent for a particular condition and determination of the antibiotic sensitivity of the identified agent. In particular, the effectiveness of individual antibiotics varies with various factors including the ability of the microorganism to resist or inactivate the antibiotic.

Despite progress in identifying methods and systems to test antibiotic susceptibility for various microorganisms, as well as the identification of related markers, determination of antibiotic susceptibility can still be challenging. In particular, determination of antibiotic susceptibility when a rapid and accurate detection is desired for microorganisms such as Neisseria gonorrhoeae which are slow growing and lack the classic transcriptional SOS response to DNA damage.

### SUMMARY

Provided herein are RNA markers of antibiotic (sometimes abbreviated as ABX) susceptibility of microorganisms and related compositions, methods and systems that can be used for their identification and/or use. In particular described herein are RNA markers and related methods and systems to test antibiotic susceptibility of microorganisms as well as RNA markers and related methods and systems for the diagnosis and/or treatment of related infections in individuals.

According to a first aspect, a method is described to identify a RNA marker of antibiotic susceptibility in a microorganism. The method comprises providing a susceptible isolate or specimen comprising a strain of the microorganism susceptible to the antibiotic and a resistant isolate or specimen comprising a strain of the microorganism resistant to the antibiotic.

The method further comprises providing a susceptible (Cs:Ts) value for a candidate marker gene in the susceptible isolate or specimen, wherein Cs is a control susceptible gene expression value Cs for a candidate marker in a control susceptible sample not treated with the antibiotic and Ts is a treated susceptible gene expression for the candidate marker in a treated susceptible sample treated with the antibiotic.

The method also comprises providing a resistant (Cr:Tr) value for a candidate marker gene in the resistant isolate or specimen, wherein Cr is a control resistant gene expression value for the candidate marker in a control resistant sample not treated with the antibiotic and Tr is a treated resistant gene expression for the candidate marker in a treated resistant sample treated with the antibiotic. The method additionally comprises selecting the candidate marker gene when Cs:Ts in the susceptible isolate or specimen is different from Cr:Tr in the resistant isolate or specimen to provide a selected marker gene expressing the RNA marker of antibiotic susceptibility of the microorganism. In particular the selected marker gene is therefore differentially expressed in the treated samples of the susceptible isolate or specimen compared with the resistant isolate or specimen as will be understood by a skilled person.

According to a second aspect, an RNA marker of antibiotic susceptibility in a microorganism, a corresponding marker gene and/or a corresponding cDNA are described, which can be obtained by the method to identify an RNA marker of antibiotic susceptibility herein described.

In some embodiments the RNA marker can be selected from a transcript encoding for a ribosomal protein of the microorganism. In some of those embodiments the RNA marker can be selected from a transcript encoding for a 30S ribosomal protein and 50S ribosomal protein. In some embodiments, the RNA marker can be selected from: a transcript of N. gonorrhoeae gene having locus tag NGO0340, a transcript of N. gonorrhoeae gene having locus tag NGO1837, a transcript of N. gonorrhoeae gene having locus tag NGO1843, a transcript of N. gonorrhoeae gene having locus tag having locus tag NGO2024, a transcript of N. gonorrhoeae gene having locus tag NGO1845, a transcript of N. gonorrhoeae gene having locus tag NGO1677, a transcript of N. gonorrhoeae gene having locus tag NGO1844, a transcript of N. gonorrhoeae gene having locus tag NGO0171, a transcript of N. gonorrhoeae gene having locus tag NGO1834, a transcript of N. gonorrhoeae gene having locus tag NGO0172, a transcript of N. gonorrhoeae gene having locus tag NGO1835, a transcript of N. gonorrhoeae gene having locus tag NGO1673, a transcript of N. gonorrhoeae gene having locus tag NGO1833, a transcript of N. gonorrhoeae gene having locus tag NGO2173, a transcript of N. gonorrhoeae gene having locus tag NGO0604, a transcript of N. gonorrhoeae gene having locus tag NGO0016, a transcript of N. gonorrhoeae gene having locus tag NGO1676, a transcript of N. gonorrhoeae gene having locus tag NGO1679, a transcript of N. gene having locus tag NGO1658 and encoding hypothetical protein, a transcript of N. gonorrhoeae gene having locus tag NGO1440, a transcript of N. gonorrhoeae gene having locus tag NGO0174, a transcript of N. gonorrhoeae gene having locus tag NGO0173, a transcript of N. gonorrhoeae gene having locus tag NGO0592, a transcript of N. gonorrhoeae gene having locus tag NGO1680, a transcript of N. gonorrhoeae gene having locus tag NGO0620, a transcript of N. gonorrhoeae gene having locus tag NGO1659, a transcript of N. gonorrhoeae gene having locus tag NGO1291, a transcript of N. gonorrhoeae gene having locus tag NGO0648, a transcript of N. gonorrhoeae gene having locus tag NGO0593, a transcript of N. gonorrhoeae gene having locus tag NGO1804, a transcript of N. gonorrhoeae gene having locus tag NGO0618, a transcript of N. gonorrhoeae gene having locus tag NGO0619, a transcript of N. gonorrhoeae gene having locus tag NGO1812, a transcript of N. gonorrhoeae gene having locus tag NGO1890, a transcript of N. gonorrhoeae gene having locus tag NGO2098, a transcript of N. gonorrhoeae gene having locus tag NGO2100 and a transcript tRNA having GeneID A9Y61_RS02445 or NGO_t12, a tRNA transcript having GeneID A9Y61_RS04515 or NGO_t15, a transcript tRNA having GeneID A9Y61_RS04510 or NGO_t14, a transcript tRNA having GeneID A9Y61_RS09170 or NGO_t37, or a transcript tRNA having GeneID A9Y61_RS00075 or NGO_t01. The locus tags and GeneIDs of the transcripts of N. gonorrhoeae gene are the locus tags and GeneIDs of the registry of locus_tag prefixes of databases of the International Nucleotide Sequence Database Collaboration (INSDC) at the filing date of the present disclosure.

According to a third aspect, a method is described to detect a transcript of an N. gonorrhoeae. The method comprises quantitatively detecting in the N. gonorrhoeae a transcript expression value of an RNA marker of N. gonorrhoeae selected from any one of the RNA markers of N. gonorrhoeae herein described, following contacting of the N. gonorrhoeae with an antibiotic to obtain an antibiotic treated transcript expression value for the RNA marker of N. gonorrhoeae

According to a fourth aspect, a method to perform an antibiotic susceptibility test for N. gonorrhoeae is described. The method comprises detecting susceptibility to an antibiotic of an N. gonorrhoeae, by quantitatively detecting in a sample comprising the N. gonorrhoeae a transcript expression value of an RNA marker of N. gonorrhoeae selected from the RNA markers of an N. gonorrhoeae herein described following contacting the sample with the antibiotic.

According to a fifth aspect a method is described to detect an RNA marker of susceptibility to an antibiotic in N. gonorrhoeae in a sample comprising the N. gonorrhoeae. The method comprises contacting the sample with the antibiotic to obtain an antibiotic treated sample and quantitatively detecting in the antibiotic treated sample one or more of the RNA marker of N. gonorrhoeae herein described.

According to a sixth aspect, a method to diagnose susceptibility to an antibiotic of a N. gonorrhoeae infection in an individual is described. The method comprises contacting with the antibiotic a sample from the individual comprising N. gonorrhoeae; and quantitatively detecting expression by the N. gonorrhoeae in the sample of a marker of antibiotic susceptibility in N. gonorrhoeae selected from any one of the transcripts of N. gonorrhoeae genes herein described. In the method, the quantitatively detecting is performed following or upon contacting the sample with the antibiotic. The method further comprises detecting whether there is a downshift of the transcript presence quantitatively detected in the antibiotic treated sample with respect to the transcript presence in a sample from the individual not treated with antibiotic and comprising N. gonorrhoeae to diagnose the antibiotic susceptibility of the N. gonorrhoeae infection in the individual.

According to a seventh aspect, a method is described to detect antibiotic susceptibility of an N. gonorrhoeae bacterium and treat N. gonorrhoeae in an individual. The method comprises contacting a sample from the individual with an antibiotic, and quantitatively detecting in the sample, expression by the N. gonorrhoeae bacteria of a marker of antibiotic susceptibility selected from any one of the transcripts of N. gonorrhoeae genes herein described. In the method, the quantitatively detecting is performed following contacting the sample with the antibiotic. The method further comprises diagnosing antibiotic susceptibility of N. gonorrhoeae infection in the individual when a downshift in expression of at least one of the detected markers in the sample is detected in comparison with a control untreated sample of the individual. The method also comprises administering an effective amount of the antibiotic to the diagnosed individual.

According to an eighth aspect, a system is described for performing at least one of the methods herein described to detect an N. gonorrhoeae transcript, to detect antibiotic susceptibility of an N. gonorrhoeae bacteria, to perform an antibiotic susceptibility test for an N gonorrhoeae, and/or to diagnose and/or treat an N. gonorrhoeae in an individual. The system comprises at least one probe specific for a transcript selected from any one of the transcripts of N. gonorrhoeae genes herein described or for a polynucleotide complementary thereof, and reagents for detecting the at least one probe.

In additional aspects, methods and systems are described, in which RNA markers and related marker genes and cDNAs of a microorganism other than N. gonorrhoeae in accordance with the second aspect of the disclosure, are used in place of N. gonorrhoeae RNA markers and related genes and cDNA to: i) detect a transcript of the another microorganism, ii) perform an antibiotic susceptibility test for the another microorganism, detect an RNA marker of susceptibility to an antibiotic in the another microorganism, diagnose susceptibility to an antibiotic of the another microorganism infection in an individual, and/or detect antibiotic susceptibility of the another microorganism and treat the another microorganism in an individual, the methods and systems comprising the features according to the third to the eighth aspect of the instant disclosure. In some of these embodiments the another microorganism is N. meningitidis.

RNA markers and related compositions methods and systems herein described allow in several embodiments to elicit in a microorganism, (e.g. N gonorrhoeae) phenotypic responses to antibiotics that are faster and greater in magnitude compared to responses in DNA markers. Therefore, in several embodiments RNA markers and related compositions methods and systems herein described allow phenotypic measurements of antibiotic susceptibility and resistance of a microorganism (e.g. N gonorrhoeae).

RNA markers and related compositions methods and systems herein described allow in several embodiments to identify as markers of antibiotic susceptibility responsive transcripts with the highest abundance and fold changes, as well as validated gene expression.

RNA markers and related compositions methods and systems herein described allow in several embodiments to perform an accurate and rapid antibiotic susceptibility test for N. gonorrhoeae based on RNA signatures.

RNA markers and related compositions methods and systems herein described allow in several embodiments to compensate for errors in sample splitting between treated and control samples and to compensate for errors in sample preparation.

RNA markers and related compositions methods and systems herein described can be used in connection with various applications wherein identification and/or detection of antibiotic susceptibility for a microorganism is desired, in particular when the microorganism is N gonorrhoeae. For example, RNA markers and related compositions methods and systems herein described can be used in drug research and to develop diagnostic and therapeutic approaches and tools to counteract infections, in particular for N gonorrhoeae. Additional exemplary applications include uses of the RNA markers and related compositions methods and systems herein described in several fields including basic biology research, applied biology, bio-engineering, aetiology, medical research, medical diagnostics, therapeutics, and in additional fields identifiable by a skilled person upon reading of the present disclosure.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and example sections, serve to explain the principles and implementations of the disclosure. Exemplary embodiments of the present disclosure will become more fully understood from the detailed description and the accompanying drawings, wherein:

FIG. 1 illustrates an exemplary workflow for selection and validation of RNA markers for phenotypic measurements of antibiotic susceptibility and resistance. Susceptible and resistant isolates of Neisseria gonorrhoeae are exposed to antibiotics (ABX) for 5, 10, and 15 min. Samples are collected for RNA sequencing at time zero and every 5 min thereafter. Genes demonstrating fold changes in expression (control:treated ratio (C:T ratio)) greater than the threshold of significance (edges of grey shaded area) are identified as differentially expressed (below grey shaded area: downregulated and above grey shaded area: upregulated). Candidate markers are selected from the pool of differentially expressed genes and validated by digital PCR.

FIGS. 2A-B shows exemplary temporal shifts in global gene expression upon ciprofloxacin exposure in Neisseria gonorrhoeae. FIG. 2A shows the distribution of -log₂(C:T ratios) for a susceptible isolate (Sus) and resistant isolate (Res) at 0, 5, 10, and 15 min. FIG. 2B shows the fold change in gene expression between control and treated samples (C:T ratio) versus expression in the control sample at 0, 5, 10, and 15 min for one susceptible isolate and one resistant isolate. Genes with C:T ratios above or below the significance threshold are identified as differentially expressed (circles with diagonal lines: downregulated; solid black circles: upregulated). In the illustration of FIGS. 2A-B, thresholds for statistical significance of fold change (black solid lines) are determined by fitting a negative exponential curve (with 90% confidence interval) to the outer edge of the -log₂ C:T ratios measured at time zero (see Methods).

FIGS. 3A-B show exemplary selection of candidate RNA markers for phenotypic antibiotic susceptibility testing in Neisseria gonorrhoeae and measurements of marker abundances per cell. FIG. 3A shows genes that are differentially expressed (dark grey) across three pairs of resistant and susceptible clinical isolates are identified as candidate markers (circles with vertical lines). Six candidate markers that span different biological functions were selected for validation (circles with diagonal lines). FIG. 3B shows copies/cell values for the candidate markers are determined from RNA sequencing and dPCR (see Methods). Data are shown for one pair of susceptible (S2) and resistant (R2) isolates at 15 min of ciprofloxacin exposure.

FIG. 4 shows an exemplary validation of the RNA sequencing approach using digital PCR (dPCR) with six candidate markers. Control:Treated ratios (C:T ratios) determined by RNA sequencing (light gray) were validated against C:T ratios measured by dPCR (dark gray). The dPCR C:T ratios were normalized using ribosomal RNA (rRNA) by dividing the C:T ratio of marker by the C:T ratio of 16S rRNA. Markers were validated using two susceptible (S1 and S2) and two resistant (R1 and R2) isolates at 15 min of ciprofloxacin exposure. In many sequencing experiments the counts per gene result from sequencing of a random sampling of the RNA pool. Relative expression values are calculated by normalizing to the total read count through generation of Transcript per Million (TPM) values (see Examples).

FIG. 5 shows in some embodiments antibiotic susceptibility testing of 49 clinical isolates using (a) porB, and (b) rpmB as RNA AST markers. Antibiotic susceptibility of 49 clinical isolates (9 susceptible and 40 resistant) from the Neisseria gonorrhoeae panel of the Central for Disease Control and Prevention (CDC) bacteria bank was determined using the “normalized” C:T ratios (C:T ratio of marker/C:T ratio of 16S rRNA). Clinical isolates were exposed to ciprofloxacin for 10 min and the concentration of RNA markers was measured by digital PCR.

FIG. 6 shows a table containing a list of candidate markers and their expression in transcripts per million (TPM) and copies per cell for susceptible isolate S2 and resistant isolate R2 after 15 min of ciprofloxacin exposure. The genome used for alignment was N. gonorrhoeae FA1090 (NCBI Reference Sequence: NC_002946.2).

FIG. 7 shows a table containing exemplary primer sequences used for validation of candidate markers by digital PCR (SEQ ID NOs: 160-173).

FIG. 8 shows a table containing minimum inhibitory concentration (MIC) values for the 49 Neisseria gonorrhoeae clinical isolates acquired from the Center for Disease Control and Prevention (CDC) and Federal Drug Administration (FDA) Antibiotic Resistance Isolate Bank published in 2018.

FIG. 9 shows a diagram reporting a fitting a curve of the C:T ratios expected to be obtained at various antibiotic concentrations in a prophetic example of the methods and systems herein described. In particular in the diagram of FIG. 9, the CT ratios obtained for a particular sample are reported vs the related concentration of antibiotic for samples comprising a microorganism susceptible to the antibiotic (black circles) a microorganism having intermediate susceptibility to the antibiotic (black squares) and a microorganism resistant to the antibiotic (black triangles). In the prophetic illustration of FIG. 9, the microorganism is N. gonorrhoeae and the antibiotic is ciprofloxacin.

### ANNEX A-E

The accompanying ANNEX A provides exemplary 16S rRNA and 23S rRNA sequences (SEQ ID NO: 1-9 and 13-27) that can be used as control transcript for normalization. ANNEX B provides exemplary marker genes (SEQ ID NO: 28-153 and 228-230) differentially expressed by an exemplary microorganism (N. gonorrhoeae) in an untreated sample and in a sample treated with an antibiotic. ANNEX C provides exemplary marker genes (SEQ ID NO: 154-159) expected to be differentially expressed by an exemplary microorganism (N. meningitidis) in an untreated sample and in a sample treated with an antibiotic. ANNEX D provides sequences of an exemplary marker of antibiotic susceptibility (porB) in 50 clinical isolates from the Center of Disease Control and Prevention (CDC) bank (SEQ ID NO: 178-227). ANNEX E provides a list of exemplary RNAs reported in Table 1 (SEQ ID NO: 231-344 and SEQ ID NO: 10-12) with a log2 fold change less than 0.32 (corresponding to <25% change) that can be used as control transcripts. ANNEX A to E which are incorporated into and constitute a part of this specification, together with the detailed description section, serve to explain the principles and implementations of the disclosure. Other features, objects, and advantages will be apparent from the entire description and drawings, and from the claims.

### DETAILED DESCRIPTION

Provided herein are RNA markers of antibiotic susceptibility of microorganisms and related compositions, methods and systems for their identification and/or use.

The term “RNA” or “Ribonucleic acid” as used herein indicates a polynucleotide composed of our of ribonucleotide bases: or an analog thereof linked to form an organic polymer. The term “ribonucleotide” refers to any compounds that consist of a ribose (ribonucleotide) sugar joined to a purine or pyrimidine base and to a phosphate group, and that are the basic structural units of a ribonucleic acid, typically adenine (A), cytosine (C), guanine (G), and uracil (U). In an RNA adjacent ribose nucleotide bases are chemically attached to one another in a chain typically via phosphodiester bonds. The term “ribonucleotide analog” refers to a ribonucleotide in which one or more individual atoms have been replaced with a different atom with a different functional group. For example, ribonucleotide analogues include chemically modified ribonucleotides, such as methylation hydroxymethylation glycosylation and additional modifications identifiable by a skilled person. Examples of chemical modifications of RNA comprise dynamic modifications to RNA identified in the transcriptome, including N⁶-methyladenosine (m⁶A), inosine (I), 5-methylcytosine (m⁵C), pseudouridine (Ψ), 5-hydroxymethylcytosine (hm⁵C), and N¹-methyladenosine (m¹A), and related epitranscriptome which are described in Song and Yi 2017,. [1] Additional chemical modifications of transfer RNA (tRNA) are described in Jackman and Alfonzo 2013 [2] (Accordingly, the term RNA includes ribonucleic acids of any length including analogs or fragments thereof.

The term “marker” as used herein refers to a category of characteristics that are objectively measured and evaluated as an indicator of biological processes, pathogenic processes, or pharmacologic response to a therapeutic intervention or an environmental exposure. A marker can be any molecule associated with the process and/or response of interest and that can be used as an identifier to detect the process and/or response of interest, such as certain characteristics in a microorganism and/or its response to a therapeutic intervention or an environmental exposure including exposure to antibiotics.

The term “antibiotic” sometimes abbreviated as ABX, as used herein refers to a type of antimicrobial used in the treatment and prevention of bacterial infection. Some antibiotics can either kill or inhibit the growth of bacteria. Others can be effective against fungi and protozoans. The term “antibiotic” can be used to refer to any substance used against microbes. Antibiotics are commonly classified based on their mechanism of action, chemical structure, or spectrum of activity. Most antibiotics target bacterial functions or growth processes. Antibiotics having bactericidal activities target the bacterial cell wall, such as penicillins and cephalosporins, or target the cell membrane, such as polymyxins, or interfere with essential bacterial enzymes, such as rifamycins, lipiarmycins, quinolones and sulfonamides. Antibiotics having bacteriostatic properties target protein synthesis, such as macrolides, lincosamides and tetracyclines. Antibiotics can be further categorized based on their target specificity. “Narrow-spectrum” antibacterial antibiotics target specific types of bacteria, such as Gram-negative or Gram-positive bacteria. “Broad-spectrum” antibiotics affect a wide range of bacteria. Exemplary antibiotics comprise topoisomerase inhibitors which are chemical compounds capable of blocking the action of a topoisomerase such as topoisomerase I and II (a type of enzyme that controls the changes in DNA structure by catalyzing the breaking and rejoining of the phosphodiester backbone of DNA strands during the normal cell cycle) and fluoroquinolones which are quinolones containing a fluorine atom in their chemical structure and are effective against both Gram-negative and Gram-positive bacteria. A quinolone antibiotic indicates any member of a large group of broad-spectrum bactericides that share a bicyclic core structure related to the compound 4-quinolone. Exemplary fluoroquinolones include ciprofloxacin (Cipro), gemifloxacin (Factive), levofloxacin (Levaquin), moxifloxacin (Avelox), norfloxacin (Noroxin), and ofloxacin (Floxin).

The wording “antibiotic susceptibility” or “antibiotic sensitivity” as used herein indicates the susceptibility of bacteria to antibiotics and the antibiotic susceptibility can vary within a species. Antibiotic susceptibility testing (AST) can be carried out to predict the clinical response to treatment and guide the selection of antibiotics as will be understood by a person skilled in the art. In some embodiments, AST categorizes organisms as susceptible, resistant, or intermediate to a certain antibiotic.

Microorganisms can be classified as susceptible (sensitive), intermediate or resistant based on breakpoint minimum inhibitory concentration (MIC) values that are arbitrarily defined and reflect the achievable levels of the antibiotic, the distribution of MICs for the organism and their correlation with clinical outcome. MIC value of a microorganism is the lowest concentration of an antibiotic that will inhibit its growth. Methods that can be used to measure the MIC of a microorganism comprise broth dilution, agar dilution and gradient diffusion (the ‘E test’), where twofold serial dilutions of antibiotic are incorporated into tubes of broth, agar plates or on a paper strip, respectively, as will be understood by a person skilled in the art. The disk diffusion method defines an organism as susceptible or resistant based on the extent of its growth around an antibiotic-containing disk. MIC values are influenced by several laboratory factors.

Laboratories follow standard for parameters such as incubation temperature, incubation environment, growth media, as well as inoculum and quality control parameters. In the U.S., standards for performing AST as well as breakpoint MIC values for various bacteria can be found in Clinical & Laboratory Standards Institute (CLSI) publications (see the web page https://clsi.org/standards/products/microbiology/documents/m100/ at the date of filing of the present disclosure). An example of standards for performing an Antibiotic Susceptibility Test (AST) as well as breakpoint MIC values for various bacteria which can be used in embodiments of the present disclosure is provided in Example 16. In Europe, standards for performing AST as well as breakpoint MIC values for bacteria can be found in European Committee on Antimicrobial Susceptibility Testing (EUCAST) see http://www.eucast.org/clinical_breakpoints/ at the time of filing of the instant disclosure) as will be understood by the skilled person.

The term “microorganism”, or “microbe” as used herein indicates a microscopic organism, which may exist in its single-celled form or in a colony of cells, such as prokaryotes and in particular bacteria.

The term “prokaryotic” is used herein interchangeably with the terms “cell” and refers to a microbial species which contains no nucleus or other organelles in the cell. Exemplary prokaryotic cells include bacteria.

The term “bacteria” or “bacterial cell”, used herein interchangeably with the terms “cell” indicates a large domain of prokaryotic microorganisms. Typically a few micrometers in length, bacteria have a number of shapes, ranging from spheres to rods and spirals, and are present in several habitats, such as soil, water, acidic hot springs, radioactive waste, the deep portions of Earth’s crust, as well as in symbiotic and parasitic relationships with plants and animals. Bacteria in the sense of the disclosure refers to several prokaryotic microbial species which comprise Gram-negative bacteria Gram-positive bacteria, Proteobacteria, Cyanobacteria, Spirochetes and related species, Planctomyces, Bacteroides, Flavobacteria, Chlamydia, Green sulfur bacteria, Green non-sulfur bacteria including anaerobic phototrophs, Radioresistant micrococci and related species, Thermotoga and Thermosipho thermophiles as would be understood by a skilled person. More specifically, the wording “Gram positive bacteria” refers to cocci, nonsporulating rods and sporulating rods, such as, for example, Actinomyces, Bacillus, Clostridium, Corynebacterium, Erysipelothrix, Lactobacillus, Listeria, Mycobacterium, Myxococcus, Nocardia, Staphylococcus, Streptococcus and Streptomyces.

The term “proteobacteria” as used herein refers to a major phylum of Gram-negative bacteria. Many move about using flagella, but some are nonmotile or rely on bacterial gliding. As understood by skilled persons, taxonomic classification as proteobacteria is determined primarily in terms of ribosomal RNA (rRNA) sequences. The Proteobacteria are divided into six classes, referred to by the Greek letters alpha through epsilon and the Acidithiobacillia and Oligoflexia, including alphaproteobacteria, betaproteobacteria and gammaproteobacteria as will be understood by a skilled person. Proteobacteria comprise the species: N. gonorrhoeae and N meningitidis within the class of Betaproteobacteria, the order: Neisseriales the Family of Neisseriaceae and the Genus of Neisseria.

In embodiments of the instant disclosure, RNA markers are described and related methods and systems to test antibiotic susceptibility of microorganisms as well as for the diagnosis and/or treatment of related infections in individuals.

In particular, in some embodiments described herein is a method to identify an RNA marker of antibiotic susceptibility in a microorganism. The method herein described is based on the use of a susceptible isolate or specimen comprising a strain of the microorganism susceptible to the antibiotic and of a resistant isolate or specimen comprising a strain of the microorganism resistant to the antibiotic.

The term “isolate” as used herein indicates a portion of matter resulting from a separation of a strain of a microorganism from a natural, usually mixed population of living microbes, as present in a natural or experimental environment, for example in water or soil flora, or from living beings with skin flora, oral flora or gut flora.

The word “specimen” as used herein indicates a portion of matter from an environment for use in testing, examination, or study. The environment can comprise living beings and in particular human beings. In these instances a specimen can include portion of tissues, organs or other biological material from the living being such as urethra, urine, cervix, vagina, rectum, oropharynges, conjunctiva, or any body fluids.

In some embodiments, the isolates can be obtained from isolate banks such as CDC and FDA AR Isolate Bank which provide curated collections of susceptible and resistant organisms. In particular in embodiments wherein the microorganism is N. gonorrhoeae, the susceptible and resistant isolates are obtained from the N. gonorrhoeae panel of the CDC Antimicrobial Resistance Isolate Bank, which as of Aug. 1, 2018 contained 50 total isolates.

In methods to identify such an RNA marker of antibiotic susceptibility in a microorganism herein described, the selected RNA marker of antibiotic susceptibility identified by the method is a transcript of a gene which is differentially expressed in a sample of the susceptible isolate or specimen treated with the antibiotic and in sample of the resistant isolate or specimen treated with the antibiotic.

The term “sample” as used herein indicates a limited quantity of something that is indicative of a larger quantity of that something, including but not limited to fluids from an isolate or a specimen such as biological environment, cultures, tissues, commercial recombinant proteins, synthetic compounds or portions thereof. In particular biological sample can comprise one or more cells of any biological lineage, as being representative of the total population of similar cells in the sampled individual. Exemplary biological samples comprise the following: cheek tissue, whole blood, dried blood spots, organ tissue, plasma, urine, mucus, mucosal secretions, vaginal fluids and secretions, urethral fluids and secretions, feces, skin, hair, or tumor cells, among others identifiable by a skilled person. Biological samples can be obtained using sterile techniques or non-sterile techniques, as appropriate for the sample type, as identifiable by persons skilled in the art. Some biological samples can be obtained by contacting a swab with a surface on a human body and removing some material from said surface, examples include throat swab, urethral swab, oropharyngeal swab, cervical swab, vaginal swab, genital swab, anal swab. Depending on the type of biological sample and the intended analysis, biological samples can be used freshly for sample preparation and analysis, or can be fixed using fixative. Preferably, in methods and systems herein described the sample comprises live cells.

The wording “differentially expressed” as used herein with respect to a gene indicates a difference in the expression of the gene by a cell under different experimental, environmental and/or biological conditions. Accordingly, differential expression of a gene can be detected in a microorganism following a different in one or more of these conditions as will be understood by a skilled person. For example, the wording “differentially expressed” can reference to a difference in the expression of a gene in a microorganism: i) with or without drug treatment, ii) on a same sample or different samples, and/or iii) at different times. Accordingly, differential expression analysis requires that gene expression values detected under the different conditions be compared and therefore that the expression of the genes be quantitatively detected.

In particular, detection of a differential expression of a gene in a susceptible or resistant isolate or specimen according to methods herein described can be performed by quantitatively detecting the expression of the gene in samples of the susceptible and resistant isolate or specimen.

The terms “detect” or “detection” as used herein indicates the determination of the existence, presence or fact of a target in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

An exemplary way to quantitatively detect differential expression is the fold change approach which can be used as a criterion to select differentially expressed genes as will be understood by a person skilled in the art. In the fold-change approach, a gene is considered to be differentially expressed if the ratio of the normalized marker expression level, possibly normalized, between the antibiotic treated and untreated conditions exceeds a certain threshold

In methods herein described, quantitative detection of expression of a gene can be performed with various techniques such as by RNA-seq, qPCR, digital PCR, and isothermal techniques such as LAMP or digital isothermal, microarrays signals, Nanostring as well high throughput RNA sequencing as reads per kilobase per million reads (RPKM) or transcripts per million (TPM) for RNA-seq data and additional nucleic acid quantification techniques identifiable to a skilled person. It should be understood that in such methods quantitative detection of expression of a gene is commonly combined with a reverse transcription step to convert the RNA sequence into a cDNA sequence which can be quantified by methods described herein and/or identifiable by a skilled person. Either sequence-specific or sequence-non-specific primers can be used to initiate reverse transcription of a target gene as will be understood by a skilled person.

In some embodiments, detecting specific gene expression can be performed at the transcription level by performing RNA-seq and calculating RNA expression values based on the sequence data.

In some embodiments, the RNA expression values can be detected and provided as transcripts per million (TPM) as will be understood by a person skilled in the art. In particular, to calculate TPM, read counts are first divided by the length of each gene in kilobases, which gives reads per kilobase (RPK). RPKs for all genes are added and the sum is divided by 1,000,000. This gives the “per million” scaling factor. Finally, the RPK value for each genes is divided by the “per million” scaling factor to give TPM. [3]

In particular, in method to identify an RNA marker of antibiotic susceptibility herein described, quantitatively detecting the expression of a gene is performed in treated samples of the susceptible and resistant isolate or specimen following treatment of the samples with the antibiotic and in control samples of the susceptible and resistant isolate or specimen without treatment with the antibiotic.

In some of these embodiments, providing a treated sample and a control sample of the susceptible and/or resistant isolate or specimen can comprise contacting a first sample of the susceptible and/or resistant isolate or specimen with a treatment media to obtain the susceptible and/or resistant control samples respectively and contacting a second sample of the susceptible and/or resistant isolate or specimen from the same source or host with the same treatment media and an antibiotic to obtain a susceptible and/or resistant antibiotic treated sample respectively. The contacting time (referring to the duration of the contact) with the treatment media is preferably substantially the same for the control sample and the treated sample. The wording “substantially the same” when referred to two or more times indicates times differing one from another of an amount up to 30%, Accordingly, for example two contacting times are substantially the same in the sense of the disclosure, if they are within approximately 30% of each other, 20% of each other, 10% of each other, 5% of each other. For example, the two contacting times can be within 2 minutes of each other, or within 1 minute of each other.

In some particular embodiments, treatment of a sample with a treatment media is performed to create a controlled environment that would minimize the impact of biochemical parameters of a sample, such as pH or salt concentration or presence of molecules other than RNA or cells (human cells or other microorganisms other than target microorganism from which gene expression is to be detected)) on the gene expression and RNA response of the target microorganism to an external stimulus such as a antibiotic treatment and/or quantitative detection of gene expression. Treatment media can be used to create a more controlled environment for obtaining a more reliable gene expression. For example, treatment media can be composed of commercially available broths designed for the cultivation of microorganisms (such as Fastidious Broth from Hardy Diagnostics) or prepared using chemically defined components. In some cases, commercial broths can be diluted to create the desired treatment environment. For example, a specific osmolarity (for example in the range 0.0 - 0.5 osmols) or pH (for example in the range 5 - 9). Treatment media can be modified to contain specific factors to increase or decrease the metabolism of the target microorganism (such as carbon source or specific anions or cations). Gentle or vigorous mixing can be performed at specific time intervals after the addition of microorganisms to the treatment media in order to maintain homogeneity and reliable gene expression.

In some embodiments, a control sample and/or treated sample of the susceptible and/or resistant isolate or specimen can preferably be pretreated to enrich said sample with RNA or with the target microorganism, and/or to remove human RNA or RNA of other microorganisms. The removal of human RNA can be performed via hybridization to beads or columns with probes specific for human RNA. The removal of human RNA can also be performed via selective lysis of human cells and degradation of released human RNA. The sample may also be pretreated to enrich or deplete, as desired, tRNA via size selection.

In some embodiments, treatment or exposure with antibiotic can be performed by adding antibiotics to the microorganism and incubating the sample under certain condition preferably following and/or upon contacting the sample with a treatment media.

Treatment media used in connection with antibiotic exposure in accordance to methods herein described can be designed to support physiological processes of the target microorganism, enable or accelerate DNA replication and translation, maintain cellular uniformity and homogeneity in suspension, and promote interaction of the microorganism and antibiotic. Accordingly, the treatment media can be selected to include a source of energy and nourishment specific for the target microorganism, such as carbon, hydrogen, oxygen, nitrogen phosphorus, Sulphur, potassium, magnesium, calcium, iron, trace elements and organic growth factors which can be provided as organic sources such as simple sugars e.g. glucose, acetate or pyruvate, amino acids, nitrogenous bases or extracts such as peptone, tryptone, yeast extract and additional identifiable by a skilled person., Inorganic sources such as ; carbon dioxide (CO2) or hydrogen carbonate salts (HCO3)NH4CI, (NH4)2S04, KNO3, and for dinitrogen fixers N2, KH2PO4, Na2HPO4, Na2SO4, H2S, KCI, K2HPO4, MgCI2, MgSO4, CaCI2, Ca(HC03)2, NaCI, FeCI3, Fe(NH4)(SO4)2, Fe-chelates1), CoCI2, ZnCI2, Na2MoO4, CuCI2, MnSO4, NiCI2, Na2SeO4, Na2WO4, Na2VO4, as well as Vitamins, amino acids, purines, pyrimidines (see the website https://www.sigmaaldrich.com/technical-documents/articles/microbiology/microbiology-introduction.html at the filing date of the present disclosure). Additional parameters considered to select the proper treatment media for a target microorganism comprise osmotic pressure, pH, oxygen content, water content, carbon dioxide content as will be understood by a skilled person to support physiological processes of the target microorganism, enable or accelerate DNA replication and translation, maintain cellular uniformity and homogeneity in suspension, and promote interaction of the microorganism and antibiotic. For example in the experiments described herein with reference to N. gonorrhoeae the treatment media used was Fastidious Broth from Hardy Diagnostics (cat no. K31) which comprise pancreatic Digest of Casein , Yeast Extract, Dextrose, Peptic Digest of Animal Tissue, Sodium Chloride, Brain Heart Infusion, TRIS , Pancreatic Digest of Gelatin, Agarose, L-Cysteine HCl, Magnesium Sulfate, Ferrous Sulfate , Hematin, NAD, Pyridoxal and Tween® 80 (see https://catalog.hardydiagnostics.com/cp_prod/content/hugo/fbbroth.htm at the filing date of the present disclosure) Additional treatment media suitable to support physiological processes of N. gonorrhoeae, to enable or accelerate DNA replication and translation, maintain cellular uniformity and homogeneity in suspension, and promote interaction of the N. gonorrhoeae and the antibiotic are identifiable by a skilled person.

In methods herein described, incubation of a sample with an antibiotic can be performed at a temperature such that a physiological response to the antibiotic is generated in the target microorganism (often the microorganisms optimal growth temperature, for example 37° C. or at a temperature ± 0.5 degrees, ± 1 degree, ± 2 degrees, ± 3° C. therefrom). Also, adding the antibiotics can be performed throughout incubation or at set intervals during incubation to increase or decrease the physiological response of the microorganism to the antibiotic.

In particular in some embodiments, the antibiotic for treating the sample herein described can be provided at a concentration equal to or above the breakpoint MIC for the susceptible isolate or specimen to the antibiotic. In particular, the antibiotic for treating the sample herein described can be provided at a concentration lower than the breakpoint MIC for the resistant isolate or specimen to the antibiotic, for example 1.5 times (or 1.5X) lower, 2 times (or 2X) lower, 3 times (or 3X) lower, 4 times (or 4X) lower, 8 times (or 8X) lower, or 16 times (or 16X) lower than the breakpoint MIC for a resistant isolate. In some embodiments, the antibiotic for treating the sample herein described is provided at a concentration higher than the breakpoint MIC for the resistant isolate or specimen to the antibiotic, for example 1.5 times (or 1.5X) higher, 2 times (or 2X) higher, 3 times (or 3X) higher, or 4 times (or 4X) higher, 8 times higher (8X), 16 times higher (or 16X) than then breakpoint MIC. The breakpoint MIC of the antibiotic can be obtained from the Clinical & Laboratory Standards Institute (CLSI) guidelines, European Committee of Antimicrobial Susceptibility Testing (EUCAST) or other sources identifiable to a skilled person. In some embodiments, samples can be treated at several concentrations of the antibiotics for example, to measure the MIC of an organism before identifying the marker of antibiotic susceptibility as will be understood by a skilled person.

In some embodiments, antibiotic treatment or exposure can be performed for a set time period (e.g. up to 5 minutes, 10 minutes, 15 minutes or 20 minutes or any other time between 0-20 minutes or longer).

In some embodiments of the methods of the instant disclosure, the time period of contacting the sample with an antibiotic is shorter than the doubling time of the target organism. For example, the time of contacting could be less than 1x doubling time, less than 0.75X doubling time, less than 0.5 doubling time, less than 0.35 doubling time, less than 0.25 doubling time, less than 0.2 doubling time, less than 0.15 doubling time, less than 0.1 doubling time, less than 0.075 doubling time, less than 0.05 doubling time.

During the incubation, the sample can be collected at different time interval for further analysis (see Example 1). In addition to collecting samples during the incubation with antibiotics, samples can be collected for analysis before treatment or exposure. Such samples can be used as controls in analysis. Detection of response of the microorganism to the antibiotic can be performed one or more times at any time after antibiotic treatment or exposure. In some embodiments, rapid detection, for example detection completed within 10 minutes, 15 minutes, 20 minutes, 30 minutes, 40 minutes after exposure.

In some of embodiments of the method to identify an RNA marker of antibiotic susceptibility herein described, providing a treated sample and a control sample of the susceptible and/or resistant isolate or specimen can comprise enriching a first sample and a second sample of the susceptible and/or resistant isolate or specimen from the same source or host with the microorganism to obtain the susceptible and/or resistant control samples respectively, and contacting the second sample with an antibiotic to obtain a susceptible and/or resistant antibiotic treated sample respectively.

In embodiments of the method to identify an RNA marker of antibiotic susceptibility herein described,, providing a treated sample and a control sample of the susceptible and/or resistant isolate or specimen can comprise enriching a first sample and a second sample of the susceptible and/or resistant isolate or specimen from the same source or host with the microorganism, contacting the first sample with a treatment media following the enriching to obtain the susceptible and/or resistant control samples respectively and contacting the second sample of the susceptible and/or resistant isolate or specimen from the same source or host with the same treatment media and an antibiotic to obtain a susceptible and/or resistant antibiotic treated sample respectively.

In methods herein described, enriching a sample with the microorganisms can be performed between sample collection (and optionally elution from a collection tool such as a swab) and exposure. In particular enriching a sample with microorganisms and in particular bacteria (such as Neisseria gonorrhoeae) can be performed by capturing the microorganism using a solid support (e.g. a membrane, a filtration membrane, an affinity membrane, an affinity column) or a suspension of a solid reagent (e.g. microspheres, beads). Capture of a target microorganism can improve the assay and the response to antibiotic. Capture can be used to enrich/concentrate low-concentration samples. Capture followed by washing can be used to remove inhibitors or components that may interfere with the method described here. Capture followed by washing may be used to remove inhibitors of nucleic acid amplification or inhibitors of other quantitative detection assays. Enrichment can also be performed using lysis-filtration techniques to lyse host cells and dissolve protein and/or salt precipitates while maintaining bacterial cell integrity then capturing target bacteria on filters (e.g. mixed cellulose ester membranes, polypropylene and polysulfone membranes). Enrichment can also be performed by binding target bacteria to membranes of microspheres, optionally coated with an affinity reagent (e.g. an antibody, an aptamer) specific to the target bacteria’s cell envelope. When microspheres or beads are used for capture, they can be filtered, centrifuged, or collected using a magnet to enrich bacteria. AST in the format described here can then be performed directly on captured bacteria, or the bacteria can be released before performing the method.

Accordingly, in methods to identify an RNA marker of antibiotic susceptibility, quantitative detection of a marker gene is performed to provide for each of the detected genes a control gene expression value C in a control sample not treated with the antibiotic and a corresponding treated gene expression value T in a treated sample treated with the antibiotic in each of the susceptible and resistant isolate or specimen.

In particular, quantitative detection of the expression of one or more genes in method herein described to identify an RNA marker of antibiotic susceptibility is performed to provide
a control susceptible gene expression value Cs for each of the detected genes in a control susceptible sample not treated with the antibiotic and a corresponding treated susceptible gene expression Ts for each of the detected genes in a treated susceptible sample treated with the antibiotic; and
a control resistant gene expression value Cr for each of the detected genes in a control resistant sample not treated with the antibiotic and a corresponding treated resistant gene expression Tr for each of the detected genes in a treated resistant sample treated with the antibiotic.

More particularly in methods to identify an RNA marker of antibiotic quantitative detection of the expression of one or more genes is performed to provide a susceptible (Cs:Ts) value for a candidate marker gene in the susceptible isolate or specimen, and a resistant (Cr:Tr) value for a candidate marker gene in the resistance isolate or specimen.

In particular providing a susceptible (Cs:Ts) value for the candidate marker gene in the susceptible isolate or specimen can be performed by
providing a treated susceptible sample treated with the antibiotic and a control susceptible sample not treated with the antibiotic,
quantitatively detecting a control susceptible gene expression value Cs for a candidate marker gene in the control susceptible sample,
quantitatively detecting a treated susceptible gene expression value Ts for the candidate marker gene in the treated susceptible sample, and
providing a susceptible (Cs:Ts) value for the candidate marker gene by dividing Cs for the candidate marker gene by Ts for the candidate marker gene .

Additionally, providing a resistant (Cr:Tr) value for the candidate marker gene in the at least one resistant isolate or specimen can be performed by.
providing a treated resistant sample treated with the antibiotic and a control resistant sample not treated with the antibiotic,
quantitatively detecting a control resistant gene expression value Cr for the candidate marker gene in the control resistant sample,
quantitatively detecting a treated resistant gene expression value Tr for the candidate marker gene in the treated resistant sample, and
providing a resistant (Cr:Tr) value for the candidate marker gene by dividing Cr for the candidate marker gene by Tr for the candidate marker gene.

In methods to identify an RNA marker of antibiotic susceptibility, the RNA is identified by selecting the candidate marker gene when Cs:Ts is different from Cr:Tr to provide a selected marker gene differentially expressed in the treated susceptible sample and in the treated resistant sample.

In some embodiments, the Cs:Ts ratio and the Cr:Tr ratios are provided by gene expressionin TPM in the control sample divided by the gene expression in TPM in the treated sample.

In some embodiments, the Cs:Ts ratio and the Cr:Tr ratios can be provided by RPKM (reads per kilobase per million mapped reads). The use of RPKM and comparison to TPM is described for example in Wagner et al 2012 [3]. In some embodiments the Cs:Ts ratio and the Cr:Tr ratios are provided by FPKM (fragments per kilobase per million), the use of FPKM is described for example in Conesa, Ana, et al. 2016 [4]. These units normalize for sequencing depth and transcript length. In some embodiments RPM (reads per million mapped reads; RPM does not normalize for transcript length) or raw sequencing read counts can be used. Typically, to calculate RPM (reads per million), the total reads from a sample are divided by 1,000,000 to obtain the “per million scaling factor”. The read counts for each gene are then divided by the “per million scaling factor” to give RPM. Also typically to calculate RPKM (for single-end RNA-seq), the RPM values are divided by the gene length in kilobases. FPKM (for paired-end RNA-seq), is calculated the same way as RPKM, taking into account that with paired-end RNA-seq, two reads can correspond to a single fragment, or, if one read in the pair did not map, one read can correspond to a single fragment as will be understood by a skilled person.

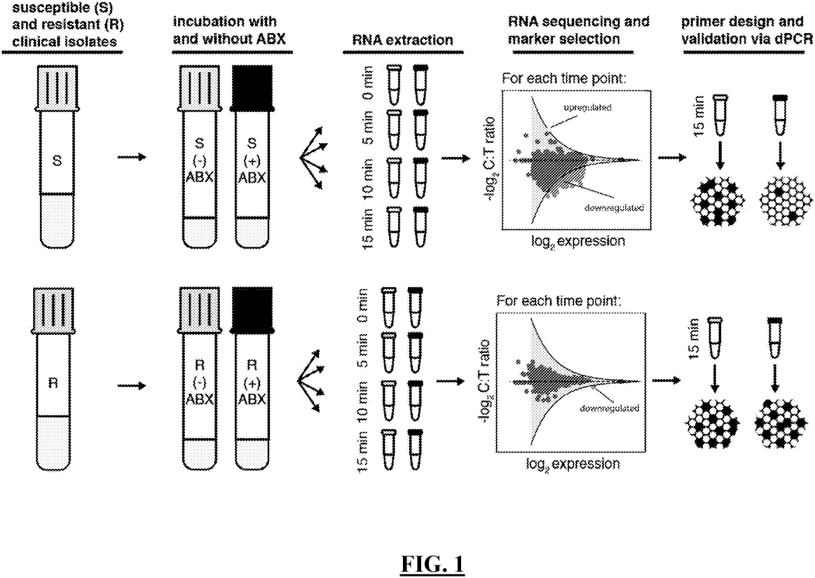

In some embodiments, the Cs:Ts ratio and the Cr:Tr ratio can be plotted as -log₂(C:T) against the -log₂(expression in TPM) for all genes (FIGS. 1-3).

In some embodiments, to qualify for a marker gene differentially expressed in the treated sample of the susceptible isolate or specimen and in the treated sample of the resistant isolate or specimen, the difference between the (Cs:Ts) value and resistant (Cr:Tr) value is statistically significant.

In preferred embodiments, to qualify for a marker gene differentially expressed in the treated sample of the susceptible isolate or specimen and in the treated sample of the resistant isolate or specimen, the difference between the (Cs:Ts) value and resistant (Cr:Tr) value is statistically significant over the related biological variability (variability due to physiologic differences among a biological unit of a same microorganism such as between different strains of the microorganism and/or between different individual microorganism of a same strains) and/or technical variability (variability due to performance of different measurements of a same biological unit), more preferably over both biological and technical variability.

To measure technical variability a Cs:Ts or a Cr:Tr ratio is measured from a given sample multiple times with the method of choice (e.g. at least 3 or more times, or 5 or more times depending on the variability of the methods chosen for measurement as will be understood by a skilled person) and statistical analysis is performed on the resulting distribution (e.g. standard error of the mean, or standard distribution depending on the number of samples used as will be understood by a skilled person). Technical variability would depend on the measurement method chosen, as different methods have different accuracy, upper quantitative limits and more importantly lower quantitative limits as will be understood by a skilled person. For example RNA sequencing and reverse transcription digital PCR are methods with low technical variability.

To measure biological variability, a Cs:Ts or a Cr:Tr ratio is measured from multiple samples (in particular one can use three resistant and three susceptible samples, or preferably at least 5 resistant and 5 susceptible samples) with a method that has minimal technical variability such as RNA sequencing or others identifiable by a skilled person upon of reading of the present disclosure.

Statistical significance can be defined using a desired percent confidence. A common choice would be a 95% confidence interval or a 99% confidence interval (for relevant descriptions see Devore 2017 [5]. Additional description of statistical analysis used in single-molecule (digital) measurements to resolve differences between two distributions is provided in Kreutz et al 2011. [6]

In preferred embodiments, to qualify for a marker gene differentially expressed in the treated sample of the susceptible isolate or specimen and in the treated sample of the resistant isolate or specimen, the difference between the (Cs:Ts) value and resistant (Cr:Tr) value is adjusted to reduce the impact of biological variability and/or technical variability, more preferably of both biological and technical variability. Accordingly, in some embodiments, the method to identify a marker, further comprises normalizing the susceptible (Cs:Ts) value and the resistant (Cr:Tr) value prior to selecting a marker gene differentially expressed in the treated samples.

The wording “normalizing” and “normalization” as used herein refer to adjustments of a value related to a quantified amount to account for variations. In particular normalization of a value can be performed to account for a variation in a parameter associated with the detection of the quantified amount, such as variations in an amount of starting material, variations in an amount of sample, variations in bacterial concentration of sample, variations due to biological variability and variations due to technical variability.

Normalizing the susceptible (Cs:Ts) value and the resistant (Cr:Tr) value is performed with a reference measurement of RNA, DNA or cell number, the number of samples, the volume of sample used, the concentration of sample used, the effective amount of sample used and/or a related ratio in a control and in a treated sample. Effective amount of sample can be calculated by for example measuring the volumes and concentration of the sample used. Normalizing the susceptible (Cs:Ts) value can be performed by dividing the control susceptible gene expression by a reference measurement in the control susceptible sample and dividing the treated susceptible gene expression by the reference measurement in the treated susceptible sample. Normalizing the resistant (Cr:Tr) value can be performed by dividing the control resistant gene expression by a reference measurement in the control resistant sample and dividing the treated resistant gene expression by the reference measurement in the treated resistant sample. In addition, the normalization ratio for susceptible sample can be calculated by dividing the control susceptible reference measurement by the treated susceptible reference measurement. Normalizing the susceptible (Cs:Ts) value can be performed by dividing the (Cs:Ts) value by a susceptible normalization ratio. The normalization ratio for resistant sample can be calculated by dividing the control resistant reference measurement by the treated resistant reference measurement. Normalizing the resistant (Cr:Tr) value can be performed by dividing the (Cs:Ts) value by a resistant normalization ratio.

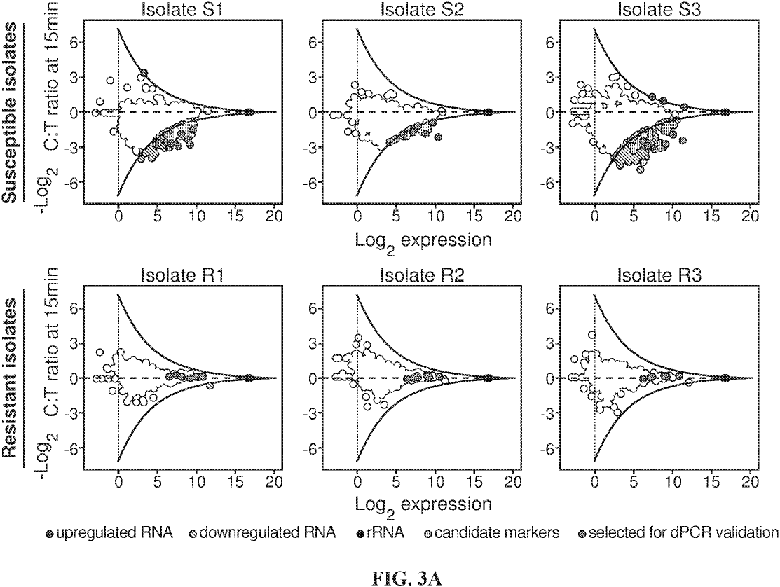

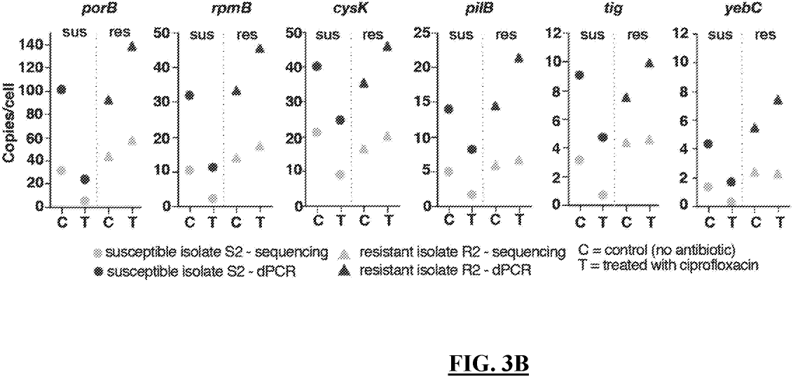

In some embodiments, normalization can be performed with reference measurement of cells such as cell number and/or a related ratio (FIGS. 3A-B).

In some embodiments of these embodiments, the reference measurement is a measurement that reflects the number of target cells. For example, prior to the calculation of a CT ratio, the RNA expression in the untreated control sample and the RNA expression in the treated sample would be divided by a cell normalization ratio between number of target cells in the treated sample and number of target cells in the control sample which can be calculated from other measurements such as optical density, turbidity, increase in intensity of a colorimetric, fluorogenic, or luminescent metabolic indicator or a live/dead indicator, colony counting after plating, amount of pathogen-specific DNA and amount of pathogen-specific RNA as will be understood by a skilled person,.

In some embodiments, normalization can be performed with reference measurement of DNA and/or a related normalization ratio.

In some of these embodiments, the reference measurement is a measurement that reflects the amount of DNA of the target pathogen. For example, the amount of DNA of the target pathogen present could be measured using real time polymerase chain reaction, digital polymerase chain reaction, digital isothermal amplification, real time isothermal amplification, and/or other nucleic acid quantification techniques described herein. One or more DNA target sequences from the genome of the target pathogen can be used for estimating the amount of DNA of the target pathogen. Preferably, DNA sequences conserved within this organism are used.

For example, prior to the calculation of the CT ratio, the RNA expression in the untreated control sample would be divided by the amount of DNA of the target pathogen measured to be present in the control sample, and the RNA expression in the treated sample would be divided by the amount of DNA of the target pathogen measured to be present in the treated sample. In addition or in the alternative prior to the calculation of the CT ratio, a DNA normalization ratio can be provided by dividing the amount of DNA of the target pathogen measured to be present in the control sample and the amount of DNA of the target pathogen measured to be present in the treated sample. The RNA expression in the untreated control sample and the RNA expression in the treated sample can then be divided by the DNA normalization ratio to normalize the related value.

In some embodiments, normalization can be performed with reference to an RNA measurement and/or a related ratio. In particular, in those embodiments, the normalization can be performed using the expression value of a reference RNA, preferably selected among RNA expressed by the microorganism with low variability among strains of the microorganism.

In some of these embodiments, prior to the calculation of a CT ratio, the RNA expression value of a marker in the treated and/or in the untreated control sample would be divided by the expression value of the reference RNA in the treated and/or untreated control sample respectively. In addition or in the alternative, prior to the calculation of a CT ratio, the RNA expression in the untreated control sample and the RNA expression in the treated sample can be divided by a RNA normalization ratio provided by the expression value of the reference RNA in the untreated control sample divided by the expression of the reference RNA in the treated sample. The expression value the reference RNA can be detected by detecting the RNA and/or the corresponding cDNA in the microorganism.

In some embodiments, also the susceptible (Cs:Ts) value and the resistant (Cr:Tr) value can be normalized with respect to a reference parameter and/or a related ratio.

For example, normalization of the susceptible (Cs:Ts) value can be performed by dividing the susceptible (Cs:Ts) value of a target transcript in an untreated control sample by the expression of a control transcript such as 16S rRNA and/or 23S rRNA in the untreated control sample, and by dividing the susceptible (Cs:Ts) value of the target transcript in the treated sample by the expression of the same control transcript (e.g. 16S rRNA and/or 23S rRNA) in the treated sample. In addition or in the alternative normalizing the susceptible (Cs:Ts) value can be performed by dividing the susceptible (Cs:Ts) value by a susceptible control (Csc:Tsc) value of a control transcript (e.g. 16S rRNA or 23S rRNA) wherein the susceptible control (Csc:Tsc) value is calculated by dividing a gene expression value of the control transcript (e.g. 16S rRNA or 23S rRNA) in the control susceptible sample by a gene expression value of the control transcript (e.g. 16S rRNA or 23S rRNA) in the treated susceptible sample. In some embodiments, the control transcript can be ribosomal rRNA such as 16S rRNA or 23S rRNA.

Normalization of the resistant (Cr:Tr) value can be performed by dividing the resistant (Cr:Tr) value of a target transcript in an untreated control sample by the expression of 16S rRNA and/or 23S rRNA in the untreated control sample, and by dividing the resistant (Cr:Tr) value of the target transcript in the treated sample by the expression of 16S rRNA and/or 23S rRNA in the treated sample. In addition or in the alternative Normalizing the resistant (Cr:Tr) value can be performed by dividing the resistant (C:T) value by a resistant control (Crc:Trc) value of a control transcript (16S rRNA or 23S rRNA) wherein the resistant control (Crc:Trc) value is calculated by dividing a gene expression value of the control transcript (16S rRNA or 23S rRNA) in the control resistant sample by a gene expression value of the control transcript (16S rRNA or 23S rRNA) in the treated resistant sample.

The term “control transcript” refers to a transcript with a fold change in gene expression between control and treated samples (C:T ratio) that is substantially the same in the resistant and susceptible samples. In some embodiments, the CT ratio of the control transcript is within a 0.1-10 range, preferably within 0.5 to 2.0 range, more preferably within 0.75 and 1.25 range.

In preferred embodiments, a control transcript is selected so that the percentage change from control to treated gene expression is less than 25%, more preferably less than 10%. For example, in some embodiments control transcripts are selected so this C:T ratio is close to 1.0 in both resistant and susceptible samples. Preferably, control transcripts are selected so this C:T ratio has low technical and biological variability, for example described by standard deviation with value of less than 0.5, less than 0.4, less than 0.3, less than 0.2, less than 0.1. In some embodiments, high-abundance transcripts (for example, transcripts in the top 10% of most expressed transcripts) are used to achieve low technical variability. Preferably, control transcripts are selected so this C:T ratio has low biological variability. Transcripts with high expression and low biological variability which are not affected by the antibiotic treatment are good candidates for control transcripts.

Exemplary RNAs with a log2 fold change less than 0.32 (corresponding to <25% change) that can be used as control transcripts is reported in Table 1 below. The fold change is calculated as the average over the six (three susceptible and three resistant) isolates sequenced. The expression guidelines follow the same as in markers.

In Table 1, the GeneID and Gene Name columns are respectively the identification or reference and name or description of the control transcript gene from NCBI FA1090. Susc. Fold Change column represents the average Log2 C:T ratio for the three susceptible isolates sequenced and Susc. Control column represents the average TPM for the three susceptible isolates sequenced.

TABLE 1

List of exemplary control RNA with low C:T ratios

| Geneid | Gene Name | DNA, cDNA and RNA sequences | Log2 Susc. Fold Change | Susc. Control |
|---|---|---|---|---|
| NGO0066a | opacity protein | SEQ ID NO: 231-233 in ANNEX E | -0.170968576 | 392.786073 |
| NGO0070 | opacity protein opA58 | SEQ ID NO: 234-236 in ANNEX E | -0.209702876 | 233.9872447 |
| NGO0372 | amino acid ABC transporter substrate-binding protein | SEQ ID NO: 237-239 in ANNEX E | 0.00367789 | 758.3587698 |
| NGO0374 | amino acid ABC transporter ATP-binding protein | SEQ ID NO: 240-242 in ANNEX E | -0.201484006 | 301.4382091 |
| NGO0399 | protease HtpX | SEQ ID NO: 243-245 in ANNEX E | -0.01276968 | 195.791542 |
| NGO0453 | type IV pilus assembly protein PilV | SEQ ID NO: 246-248 in ANNEX E | -0.304641252 | 102.3440114 |
| NGO0571 | hypothetical protein | SEQ ID NO: 249-251 in ANNEX E | -0.036612448 | 150.1617177 |
| NGO0632 | Fe-S)-cluster assembly protein | SEQ ID NO: 252-254 in ANNEX E | 0.020285087 | 284.094415 |
| NGO0633 | iron-sulfur cluster assembly scaffold protein | SEQ ID NO: 255-257 in ANNEX E | 0.005118807 | 205.6161095 |
| NGO0678 | hypothetical protein | SEQ ID NO: 258-260 in ANNEX E | -0.289054194 | 140.5781892 |
| NGO0926 | peroxiredoxin family protein/glutaredoxin | SEQ ID NO: 261-263 in ANNEX E | -0.206313513 | 1041.933939 |
| NGO0936 | elongation factor P | SEQ ID NO: 264-266 in ANNEX E | -0.139160962 | 220.8383642 |
| NGO0950a | opacity protein | SEQ ID NO: 267-269 in ANNEX E | -0.023532111 | 210.4893677 |
| NGO1040a | opacity protein | SEQ ID NO: 270-272 in ANNEX E | -0.230149948 | 366.4512778 |
| NGO1073a | opacity protein | SEQ ID NO: 273-275 in ANNEX E | -0.071137135 | 260.6204103 |
| NGO_r02 | 23S ribosomal RNA | SEQ ID NO: 324-326 in ANNEX E | 0.05763481 | 98595.48132 |
| NGO_r03 | 16S ribosomal RNA | SEQ ID NO: 327-329 in ANNEX E | 0.075840851 | 09877.3255 |
| NGO1225 | peptidyl-prolyl isomerase | SEQ ID NO: 276-278 in ANNEX E | -0.269241544 | 210.4315667 |
| NGO1277a | opacity protein | SEQ ID NO: 279-281 in ANNEX E | -0.22403411 | 774.1660932 |
| NGO_r05 | 23S ribosomal RNA | SEQ ID NO: 330-332 in ANNEX E | 0.056291313 | 105513.464 |
| NGO_r06 | 16S ribosomal RNA | SEQ ID NO: 333-335 in ANNEX E | 0.075380208 | 109869.8039 |
| NGO1513 | opacity protein OpaD | SEQ ID NO: 282-284 in ANNEX E | 0.219592047 | 467.3481454 |
| NGO1553a | opacity protein | SEQ ID NO: 285-287 in ANNEX E | 0.046438846 | 199.2181833 |
| NGO_r08 | 23S ribosomal RNA | SEQ ID NO: 336-338 in ANNEX E | 0.055364901 | 113073.5889 |
| NGO_r09 | 16S ribosomal RNA | SEQ ID NO: 339-341 in ANNEX E | 0.077151808 | 109869.6765 |
| NGO1762 | acpP | SEQ ID NO: 288-290 in ANNEX E | 0.108200968 | 418.8600381 |
| NGO1842 | Tuf | SEQ ID NO: 291-293 in ANNEX E | 0.124500308 | 948.5850148 |
| NGO_t45 | tRNA-Trp | SEQ ID NO: 318-320 in ANNEX E | 0.08302137 | 658.3452457 |
| NGO_t47 | tRNA-Gly | SEQ ID NO: 321-323 in ANNEX E | 0.292430237 | 660.2453417 |
| NGO1871 | Peptide deformylase | SEQ ID NO: 294-296 in ANNEX E | 0.046048547 | 201.2627628 |
| NGO_r11 | 23S ribosomal RNA | SEQ ID NO: 342-344 in ANNEX E | 0.059281774 | 109175.2904 |
| NGO_r12 | 16S ribosomal RNA | SEQ ID NO: 10-12 in ANNEX E | 0.080622568 | 109611.2649 |
| NGO1908 | pilus retraction protein PilT | SEQ ID NO: 297-299 in ANNEX E | -0.17727476 | 302.6151055 |
| NGO1982 | hypothetical protein | SEQ ID NO: 300-302 in ANNEX E | 0.181935722 | 123.4307398 |
| NGO2060a | opacity protein | SEQ ID NO: 303-305 in ANNEX E | 0.014421302 | 237.2782045 |
| NGO2084 | membrane protein | SEQ ID NO: 306-308 in ANNEX E | 0.242929753 | 158.6654013 |
| NGO2134 | rpsU | SEQ ID NO: 309-311 in ANNEX E | 0.171806238 | 710.1798633 |
| NGO2145 | ATP synthase subunit C | SEQ ID NO: 312-314 in ANNEX E | 0.08725091 | 432.1571821 |
| NGO2146 | ATP synthase subunit B | SEQ ID NO: 315-317 in ANNEX E | 0.188542222 | 274.7458871 |

In some embodiments, the control transcript can be a ribosomal RNA, including 23S rRNA, 16S rRNA, 5S rRNA and other RNA component of ribosome.

In some embodiments, 16S rRNA or 23 rRNA are used as control transcripts for normalization. Exemplary control transcripts are listed in Table 2:

TABLE 2

List of exemplary 16S ribosomal RNA and 23S ribosomal RNA used as control transcripts for normalization

| Locus Tag | Gene Description | DNA, cDNA and RNA sequences | Average Fold Change | Average Relative Abundance (TPM) |
|---|---|---|---|---|
| A9Y61_RS06450 or NGO_r02 | 23S ribosomal RNA | SEQ ID NOs: 1 to 3 in ANNEX A | 1.054 | 110136.512 |
| A9Y61_RS06465 or NGO_r03 | 16S ribosomal RNA | SEQ ID NOs:4 to 6 in ANNEX A | 1.048 | 99551.420 |
| A9Y61_RS07175 or NGO_r05 | 23S ribosomal RNA | SEQ ID NOs:7 to 9 in ANNEX A | 1.054 | 110037.026 |
| A9Y61_RS07190 or NGO_r04 | 16S ribosomal RNA | SEQ ID NOs:13 to 15 in ANNEX A | 1.050 | 105158.011 |
| A9Y61_RS09315 or NGO_r08 | 23S ribosomal RNA | SEQ ID NOs:16 to 18 in ANNEX A | 1.054 | 110108.563 |
| A9Y61_RS09330 or NGO_r09 | 16S ribosomal RNA | SEQ ID NOs:19 to 21 in ANNEX A | 1.048 | 105226.036 |
| A9Y61_RS 10490 Or NGO_r11 | 23S ribosomal RNA | SEQ ID NOs:22 to 24 in ANNEX A | 1.054 | 110097.800 |
| A9Y61_RS 10505 or NGO_r12 | 16S ribosomal RNA | SEQ ID NOs:25 to 27 in ANNEX A | 1.048 | 05322.731 |

In some embodiments, control transcript according to the instant disclosure can have a sequence identity of at least 80%, or 90%, up to 100% of the markers listed in Table 1 and 2. In particular markers of the instant disclosure can be have sequence identity of 93%, 94%, 95%, 96%, 97%, 98%, or 99% of the sequences indicated in Tables 1 and 2.

The Gene IDs listed above as well as their sequences can be retrieved from NCBI database (https://www.ncbi.nlm.nih.gov/nuccore/1036099588) as will be understood by a person skilled in the art.

For example, in some embodiments, a specific region (such as a gene) of the DNA can be measured in in the control and treated sample and used as normalization DNA measurement, as will be understood by a skilled person. In some embodiments DNA normalization methods can be performed by PCR or dPCR. In some embodiments, a fluorescence dye that quantitatively stains DNA can be used as a normalization method. Additional methods to perform normalization DNA measurements are identifiable by a skilled person upon reading of the present disclosure.

In some embodiments, quantitatively detecting Cs Ts and Cr and Tr can be performed on a treated sample and corresponding control sample under several sets of conditions (e.g. varying treatment times, different experimental settings and/or using a plurality of isolates or specimen and/or a plurality of related control and/or treated sample) to provide a gene expression pattern for the candidate marker gene formed by the gene expression values detected in each treated and corresponding control samples under each set of conditions. In those embodiments, the differential expression of the candidate gene marker is detected with respect to the corresponding gene expression pattern according to approaches identifiable by a skilled person upon reading of the present disclosure.

In some embodiments, the candidate gene marker is a plurality of candidate gene markers. In those embodiments the quantitative detection of the related expression can be performed by detecting global gene expression, or patterns of gene expression, in the samples of the susceptible and resistant isolate or specimen.

The wording “global gene expression” as used herein indicates an expression level of a population of RNA molecules in cells and tissues. In particular, global gene expression can be performed to detect a transcriptome which is the set of all RNA molecules in one cell or a population of cells. Global gene expression is an approach typically used to investigate a transcriptional behavior of a biological system in connection with various biological phenomenon, as global genes expression can provide quantitative information about the population of RNA species in cells and tissues. The wording “Pattern of gene expression” refers to gene expression of multiple markers, or gene expression of the same marker over multiple conditions.

In embodiments herein described detecting global gene expression and pattern of gene expression can be performed using DNA microarrays, Nanostring, RNA-Seq, digital PCR, bulk qPCR, isothermal techniques such as LAMP or digital isothermal amplification techniques, and other nucleic acid quantification techniques described herein to measure the levels of RNA species in biological systems.

In those embodiments, providing a susceptible (Cs:Ts) value for the candidate marker gene in the susceptible isolate or specimen and providing a resistant (Cr:Tr) value for the candidate marker gene in the resistant isolate or specimen can be performed by
quantitatively detecting a control susceptible gene expression value Cs for each of the plurality of genes in the control susceptible sample and a control resistant gene expression value Cr for each of a plurality of genes in the control resistant sample,
quantitatively detecting a treated susceptible gene expression Ts value for each of the plurality of genes in the treated susceptible sample and a treated resistant gene expression value Tr for each of a plurality of genes in the treated resistant sample,
providing a susceptible (Cs:Ts) value and a corresponding resistant (Cr:Tr) value for each of the plurality of genes.

In those embodiments, the method further comprises selecting a set of maker genes differentially expressed in the treated sample of the susceptible isolate or specimen and in the treated sample of the resistant isolate or specimen by identifying the genes with the susceptible (Cs:Ts) value different from the corresponding resistant (Cs:Ts) value.

In some embodiments, to qualify for a marker gene differentially expressed in the treated sample of the susceptible isolate or specimen and in the treated sample of the resistant isolate or specimen, the difference between the susceptible (C:T) value and resistant (C:T) value is larger than a threshold.

In some embodiments, the method further comprises selecting the candidate gene markers having a Cs:Ts and/or Cr:Tr above or below a threshold of significance respectively. In some embodiments, an individual threshold is established for each of the plurality of markers in accordance with approaches of the present disclosure. In particular the threshold can be based on the knowledge of a distribution of a parameter indicative of the expression of one or more transcripts, to include transcripts differentially expressed in treated vs control sample across the distribution. For example to establish the threshold for each marker, C:T measurements are performed on a plurality of resistant and susceptible isolates, optionally including isolates with intermediate resistance. Threshold values can then be chosen to maximally separate C:T ratios for resistant and susceptible isolates. If a plurality of markers is used to determine antibiotic susceptibility of an organism, a number of algorithms can be used to interpret such information to make the determination. For example, weighted average or weighted sum of C:T ratios of the markers can be compared to the weighted average or weighted sum of the thresholds. Machine learning and pattern-recognition algorithms can be used. Measured fold-changes can be multiplied and compared to multiplied thresholds for multiple markers.

In detections when there is overlap between C:T ratios of resistant and susceptible isolates, various classification models can be used to map the C:T ratios between the susceptible and resistant groups. For example, receiver operating characteristic (ROC curve) can be analyzed and used to set optimal threshold. (see https://en.wikipedia.org/wiki/Receiver_operating_characteristic at the filing date of the present disclosure). ROC curve can be used to select optimal balance of analytical specificity and sensitivity of the test. In particular, the wording “analytical sensitivity” indicates the method’s ability to detect the target molecule at low levels in a sample. This is defined as the lowest concentration of RNA in a sample that can be detected >95% of the time. The wording “analytical specificity” indicates the method’s ability to detect the intended target in a complex sample. This refers to the ability of the method to differentiate between the intended target and similar targets from other bacterial species and the ability of the method to overcome inhibitors from the sample. When tested with clinical samples, ROC curve can be used to select optimal balance of clinical specificity and sensitivity of the test. Furthermore, prevalence data can be incorporated to provide a further refinement or predicted specificity and sensitivity of the test.

Additionally, in those embodiments detection where there is overlap between C:T ratios of resistant and susceptible isolates, the threshold can be also set in view of the severity of one type of error versus another, to reduce or minimize major errors even if this requires an increase of minor errors. For example, in case of overlaps between C:T ratios of resistant and susceptible isolates the threshold can be set to reduce up to minimize false susceptible (considered a more problematic error in terms of resulting treatment) increasing the expected percentage of false resistant. In some embodiments, the method can be performed with a plurality of susceptible and/or resistant isolates having genetic variability.

The wording “genetic variability” refers to either the presence of, or the generation of, genetic differences in a microorganism. The term “genetic variability” is defined as the formation of individuals differing in genotype, or the presence of genotypically different individuals. Therefore, Genetic variability refers to the difference in genotype between specific organisms while biological variability refers to the phenotypic differences between specific organisms, in this case RNA response to an antibiotic given for a specified amount of time.

Accordingly, a genetic variant indicates a genetic difference from a reference genome. The genetic variant can be used to describe an alteration (such as insertions, deletions, and /or replacement of nucleotides) that can be a result of mutations, recombination as will be understood by a person skilled in the art. Exemplary genetic variants comprise single base-pair substitution, also known as single nucleotide polymorphism (SNP), insertion or deletion of a single stretch of DNA sequence that can range for example from two to hundreds of base-pairs in length, and structural variation including copy number variation and chromosomal rearrangement events. The structural variation typically include deletion, insertion, inversion, duplication and copy number variation of the individual nucleic acids as will be understood by a person skilled in the art.

In particular in some embodiments, the susceptible and resistant isolates or specimen used herein for identifying a marker of antibiotic susceptibility comprise at least three different susceptible isolates or specimen and at least three different resistant isolates or specimen, preferably at least five different susceptible isolates or specimen and at least five different resistant isolates.

In preferred embodiments, the susceptible and resistant isolates or specimen used herein for identifying a marker of antibiotic susceptibility are selected to differ in genotypes and in biological responses to antibiotic administration to maximize genetic and biological variability of the isolates or specimen used for identifying a marker.

In some embodiments, selection of susceptible and resistant isolates or specimen used for identifying a marker of antibiotic susceptibility to increase or maximize genetic variability can be performed by sequencing the genomes of multiple isolates and selecting genetically different isolates or by obtaining isolates from different clusters from an isolate depository such as the CDC isolate bank or others entities identifiable by a skilled person. Hierarchical clustering based on genetic distance can be performed by first generating a SNP profile for each isolate against a reference genome (NCBI FA1090). Then a maximum-likelihood based inference method for phylogenetic tree generation can be performed to cluster isolates by genetic variability using tools such as RAxML or Garli and additional tools identifiable by a skilled person. Isolates can then be chosen from a plurality of clusters after hierarchical phylogenetic clustering.

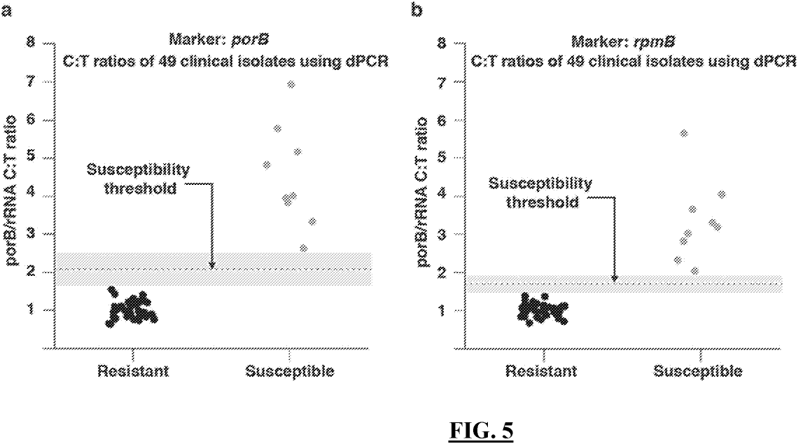

In some embodiments, selection of susceptible and resistant isolates or specimen used for identifying a marker of antibiotic susceptibility to increase or maximize biological variability in RNA expression can be performed on a full transcriptome scale, (e.g. by detecting the transctiptome through RNA sequencing or on a gene specific scale (e.g. by detecting the specific gene expression through PCR based methods) following administration of an antibiotic and then calculating the related C:T ratio. Reference is made in this connection to the resistant isolates in FIG. 5 of the instant disclosure wherein detection of the spread of the porB C:T ratios would provide an estimate for the biological variability for porB. Additional indicator of biological variability comprise resistance profile to antibiotics indicated for example in terms of MIC for one or more antbiotics.

In preferred embodiments, selection of susceptible and resistant isolates or specimen used herein for identifying a marker of antibiotic susceptibility to select isolates having a high prevalence in a target region (area where the marker is intended to be used, such a city a county, a state, a country or larger regions formed by groups of countries or the entire world) based on surveys or other epidemiological data on the strains of a certain microorganism in the target region. In particular, one or more isolates can be selected that cluster together with strains accounting for at least 75% more preferably at least 85% even more preferably at least 90% or most preferably at least 95% of the strains infecting individuals in the target region.

In preferred embodiments selection of susceptible and resistant isolates or specimen used for identifying a marker of antibiotic susceptibility is performed by selecting at least 3 to 5 isolates maximizing genetic variability, biological variability while selecting the isolates with a prevalence of at least 75% more preferably at least 85% even more preferably at least 90% or most preferably at least 95% of the strains infecting individuals in a target region.

Following selection of a plurality of isolates preferably maximizing genetic and biological variability and prevalence in a target region, candidate markers can be tested with methods herein described.

In some embodiments, detecting expression of a candidate gene marker in a plurality of the selected susceptible isolates and in a plurality of the selected resistant isolates (at least three preferably at least 5) gene expression upon antibiotic exposure is performed by detecting expression a plurality of candidate gene markers (e.g. at least 2, at least 5, at least 10, at least 50 or, at least 100 or 300 or more depending on the genome size and the candidate markers selected and the detection technique selected). In those embodiments, detecting expression a plurality of candidate gene markers can be performed by detecting patterns of gene expression and/or global gene expression upon antibiotic exposure in a control sample and in a treated sample of each of the plurality of the selected susceptible isolates and in each of the plurality of the selected resistance isolates.

In some embodiments wherein quantitatively detecting expression of a candidate marker genes is performed by quantitatively detecting a plurality of candidate marker gene, and/or by quantitatively detecting expression of a candidate marker gene in a plurality of resistant and/or susceptible isolate, the method to identify a marker of antibiotic susceptibility in a microorganism of the instant disclosure can further comprises selecting the candidate gene marker with a transcript having a high fold change in expression upon antibiotic exposure.

A high fold change is defined as at least two folder change or higher. In particular, in some embodiments, a significant shift of fold change (larger than 4) in transcript levels can be observed within 5 min of antibiotic exposure. In some typically more infrequent instances genes can respond to antibiotic exposure with changes as large as 6-fold within 5 min.

The term “transcript” as used herein refers to any ribonucleic acid sequence provided in the microorganism without limitation to any specific type, function or length. Transcripts include messenger RNA (mRNA), transfer RNA (tRNA), and ribosomal RNA (rRNA) of any length.

In some embodiments, the method to identify an RNA marker of antibiotic susceptibility further comprises validating the candidate markers by determining whether the candidate markers respond consistently across a large pool of isolates with genetic variability.

The validation of candidate markers can be performed by selecting the candidate markers with the highest abundance and fold change and using these selected candidate markers to determine the susceptibility of clinical isolates with known susceptibility/resistance. The clinical isolates can be obtained from the Centers for Disease Control (CDC) Antimicrobial Resistance Isolate Bank (see Example 10) and preferably represent a large degree of genetic variation or difference.

Validate markers are identified as markers showing consistency in their ability to correctly determine susceptibility or resistant of the clinical isolates.

In some embodiments, wherein quantitatively detecting expression of a candidate marker genes is performed by quantitatively detecting a plurality of candidate marker gene, and/or by quantitatively detecting expression of a candidate marker gene in a plurality of resistant and/or susceptible isolates or specimen, the method to identify a marker of antibiotic susceptibility in a microorganism of the instant disclosure further comprises selecting a candidate gene marker having transcripts representative of different biochemical pathways.

The term “biochemical pathways” refer to a sequence of chemical or biochemical reactions catalyzed by enzymes in which a product of one enzyme acts as the substrate for the next.

In some embodiments of the method to identify a marker of antibiotic susceptibility in a microorganism of the instant disclosure, the microorganism is a slow growing microorganism, a microorganism with a transcriptome which is not characterized and/or a microorganism that lacks a transcriptional SOS response to DNA damage.

The term “slow growing” as used herein indicates an organism with a doubling time longer than 30 minutes.

In some embodiments of the method to identify a marker of antibiotic susceptibility in a microorganism of the instant disclosure, the antibiotic is a fluoroquinolone. The term “fluoroquinolone” as used herein indicates a group of antibiotics containing a fluorine atom in their chemical structure. Fluoroquinolones are usually effective against both Gram-negative and Gram-positive bacteria. Exemplary fluoroquinolone include levofloxacin, ofloxacin, gatifloxacin, moxifloxacin, and norfloxacin.

In some of these embodiments, the antibiotic for treating the sample herein described, the concentration of the antibiotic can be provided at a concentration between 0.015 microgram/mL and 16.0 microgram/mL.

In some of these embodiments, the fluoroquinolones is ciprofloxacin. In some of these embodiments, the concentration of antibiotic used during exposure or treatment can be any concentration between the susceptible and resistant MIC breakpoints of the target organism. For example, for exposure or treatment of Ng with ciprofloxacin, the concentration of antibiotic used could any concentration ≥ 0.06 microgram/mL (the susceptible MIC breakpoint for ciprofloxacin for Neisseria gonorrhoeae) and ≤ 1.00 microgram/mL (the resistant MIC breakpoint for ciprofloxacin for Neisseria gonorrhoeae). In some embodiments, for example when faster responses are desired, higher than breakpoint concentrations can be used.

In some embodiments of the method to identify a marker of antibiotic susceptibility in a microorganism of the instant disclosure the antibiotic is an antibiotic inhibiting the enzymes topoisomerase II (DNA gyrase) and topoisomerase IV, thereby inhibiting cell division. Examples include Aminocoumarin antibiotics such as Novobiocin, Albamycin Coumermycin, Clorobiocin, and their derivatives, Simocyclinones and derivatives, moxifloxacin, ciprofloxacin, azithromycin, tetracycline, and ceftriaxone.

Additional examples of such antibiotics comprise novel bacterial topoisomerase inhibitors (NBTIs) and in particular Type I NBTIs such as gepotidacin and its analogues, GSK945237, AM-8722, 1,5-naphthyridine oxabicyclooctane linked NBTIs, and type II NBTIs, such as quinolone pyrimidone trione-1 (QPT-1) Zoliflodacin (AZD0914), isothiazolone analogue REDX04957 and its two enantiomer forms, REDX05967 and REDX05990,. Further examples comprise nalidixic acid, oxolinic acid, norfloxacin, iprofloxacin, levofloxacin, moxifloxacin, Gemifloxacin, EDX04139, REDX05604, REDX05931, kibdelomycin thiosemicarbazide; 4,5-dibromo-N-(thiazol-2-yl)-1H-pyrrole-2-carboxamide, cyclothialidine; pyrazolopyridone, 4-(4-(3,4-dichloro-5-methyl-1H-pyrrole-2carboxamido), piperidin-1-yl)-4-oxobutanoic acid, trans-4-(4,5-dibromo1H-pyrrole-2-carboxamide)cyclohexyl)glycine, pyrazolopyridones, cyclothialidines and their analogues, GR122222X, cinodine, albicidin, clerocidin, microcin B17, CcdB, an pentapeptide repeat proteins Qnr and MfpA, as well as additional antibiotics identifiable by a skilled person (see e.g. Badshah and Ullah 2018 [7] and Collin et al. 2018 [8]).

In the instant disclosure, an RNA marker of antibiotic susceptibility in a microorganism is described, as well as a corresponding marker gene and/or a corresponding cDNA are described, which can be obtained by the method to identify an RNA marker of antibiotic susceptibility

In some embodiments, the RNA markers comprise RNA markers encoding a ribosomal protein. The term “ribosomal protein” is the protein component of ribosome that in conjunction with rRNA make up the ribosomal subunits involved in the cellular process of translation. Prokaryotic bacteria and archaea have a 30S small subunit and a 50S large subunit. Accordingly, some of these mRNA markers disclosed herein comprise mRNA markers encoding 50S ribosomal proteins and mRNA markers encoding 30S ribosomal proteins.

Exemplary mRNA markers encoding ribosomal proteins include mRNA encoding 50S L4, 50S L13, 30S S12, 50S L27, 50S L19, 30S S19, 50S L2, 50S L22, 50S L32, 30S S1, 50S L21, 50S L33, 30S S16, 50S L28.

An additional list of exemplary mRNA markers of N. gonorrhoeae encoding ribosomal proteins is also shown in Table 5 of the instant application including rplD, rplM, rpsL, rpmA, rplS, rpsS, rplB, rplV, rpmF, rpsA, rplU, rpmG, rpsP, and rpmB.In some embodiments of the method herein described to identify a marker of antibiotic susceptibility in a microorganism of the instant disclosure, the microorganism is N. gonorrhoeae.

Neisseria gonorrhoeae is one type of proteobacteria that causes the sexually transmitted genitourinary infection gonorrhea as well as other forms of gonococcal disease including disseminated gonococcemia, septic arthritis, and gonococcal ophthalmia neonatorum. The term “Neisseria gonorrhea” includes all strains of N. gonorrhoeae identifiable by a person skilled in the art. Neisseria gonorrhea also includes genetic variants of different strains. One may determine whether the target organism is N. gonorrhoeae by a number of accepted methods, including sequencing of the 16S ribosomal RNA (rRNA) gene, as described in Chakravorty et al (2007) for N. gonorrhoeae. [9]

In some embodiments of the method herein described to identify a marker of antibiotic susceptibility in a microorganism of the instant disclosure, the microorganism is Neisseria meningitidis. Neisseria meningitidis, often referred to as meningococcus, is a Gram-negative bacterium that can cause meningitis and other forms of meningococcal disease such as meningococcemia, a life-threatening sepsis.

In some embodiments of the method herein described to identify a marker of antibiotic susceptibility in a microorganism of the instant disclosure, the RNA marker is not a direct target of the antibiotic. For example in some embodiments where the antibiotic is a quinolone and in particular ciprofloxacin, the selected markers are not identified target of gyrA, parC and/or recA identified as target for ciprofloxacin.

In some embodiments of the instant disclosure wherein the microorganism is N. gonorrhoeae, the markers can be selected from: a transcript of N. gonorrhoeae gene having locus tag NGO0340, a transcript of N. gonorrhoeae gene having locus tag NGO1837, a transcript of N. gonorrhoeae gene having locus tag NGO1843, a transcript of N. gonorrhoeae gene having locus tag having locus tag NGO2024, a transcript of N. gonorrhoeae gene having locus tag NGO1845, a transcript of N. gonorrhoeae gene having locus tag NGO1677, a transcript of N. gonorrhoeae gene having locus tag NGO1844, a transcript of N. gonorrhoeae gene having locus tag NGO0171, a transcript of N. gonorrhoeae gene having locus tag NGO1834, a transcript of N. gonorrhoeae gene having locus tag NGO0172, a transcript of N. gonorrhoeae gene having locus tag NGO1835, a transcript of N. gonorrhoeae gene having locus tag NGO1673, a transcript of N. gonorrhoeae gene having locus tag NGO1833, a transcript of N. gonorrhoeae gene having locus tag NGO2173, a transcript of N. gonorrhoeae gene having locus tag NGO0604, a transcript of N. gonorrhoeae gene having locus tag NGO0016, a transcript of N. gonorrhoeae gene having locus tag NGO1676, a transcript of N. gonorrhoeae gene having locus tag NGO1679, a transcript of N. gonorrhoeae gene having locus tag NGO1658, a transcript of N. gonorrhoeae gene having locus tag NGO1440, a transcript of N. gonorrhoeae gene having locus tag NGO0174, a transcript of N. gonorrhoeae gene having locus tag NGO0173, a transcript of N. gonorrhoeae gene having locus tag NGO0592, a transcript of N. gonorrhoeae gene having locus tag NGO1680, a transcript of N. gonorrhoeae gene having locus tag NGO0620, a transcript of N. gonorrhoeae gene having locus tag NGO1659, a transcript of N. gonorrhoeae gene having locus tag NGO1291, a transcript of N. gonorrhoeae gene having locus tag NGO0648, a transcript of N. gonorrhoeae gene having locus tag NGO0593, a transcript of N. gonorrhoeae gene having locus tag NGO1804, a transcript of N. gonorrhoeae gene having locus tag NGO0618, a transcript of N. gonorrhoeae gene having locus tag NGO0619, a transcript of N. gonorrhoeae gene having locus tag NGO1812, a transcript of N. gonorrhoeae gene having locus tag NGO1890, a transcript of N. gonorrhoeae gene having locus tag NGO2098, a transcript of N. gonorrhoeae gene having locus tag NGO2100, a transcript tRNA having GeneID A9Y61_RS02445 or NGO_t12, a transcript tRNA having GeneID A9Y61_RS04515 or NGO_t15, a transcript tRNA having GeneID A9Y61_RS04510 or NGO_t14, a transcript tRNA having GeneID A9Y61_RS09170 or NGO_t37, and a transcript tRNA having GeneID A9Y61_RS00075 or NGO_t01. The sequences of these transcripts can be retrieved from the public databases in compliance with the International Nucleotide Sequence Database Collaboration at the date of filing of the present disclosure as will be understood by a person skilled in the art. In particular, the sequences of these transcript can be identified by entering the locus tag or the GenID, alone or in combination with additional information provided in the present disclosure, in databases such as National Center for Biotechnology Information (NCBI) the European Bioinformatics Institute (EMBL-EBI) and DNA Data Bank of Japan (DDBJ) at the date of filing of the present disclosure.

In some embodiments the cDNAs of N. gonorrhoeae can have a sequence that can be shorter or longer than the sequences in the databases as will be understood by a skilled person. In particular, the transcript can include a re be up to 30 bp, 40 bp, 50 bp, 60 bp, 70 bp, 80 bp, 90 bp, 100 bp, 150 bp, 200 bp, 250 bp, 300 bp, 400 bp, 500 bp, 750 bp, 1000 bp, 1500 bp, 2000 bp, 2500 bp , or up to 3000 bp, shorter or longer of the sequence in the database as will be understood by a skilled person. Exemplary sequences for the above markers are provided in Table 3 below.

TABLE 3

List of exemplary marker genes differentially expressed between an untreated sample and a sample treated with antibiotics.

| | |
|---|---|
| Locus Tag | DNA, cDNA and RNA Sequences |
| NGO0340 | SEQ ID Nos. 49 to 51 in ANNEX B |
| NGO1837 | SEQ ID Nos. 109 to 111 in ANNEX B |
| NGO1843 | SEQ ID Nos. 112 to 114 in ANNEX B |
| NGO2024 | SEQ ID Nos. 124 to 126 in ANNEX B |
| NGO1845 | SEQ ID Nos. 118 to 120 in ANNEX B |
| NGO1677 | SEQ ID Nos. 91 to 93 in ANNEX B |
| NGO1844 | SEQ ID Nos. 115 to 117 in ANNEX B |
| NGO0171 | SEQ ID Nos. 37 to 39 in ANNEX B |
| NGO1834 | SEQ ID Nos. 103 to 105 in ANNEX B |
| NGO0172 | SEQ ID Nos. 40 to 42 in ANNEX B |
| NGO1835 | SEQ ID Nos. 106 to 108 in ANNEX B |
| NGO1673 | SEQ ID Nos. 85 to 87 in ANNEX B |
| NGO1833 | SEQ ID Nos. 100 to 102 in ANNEX B |
| NGO2173 | SEQ ID Nos. 136 to 138 in ANNEX B |
| NGO0604 | SEQ ID Nos. 58 to 60 in ANNEX B |
| NGO0016 | SEQ ID Nos. 34 to 36 in ANNEX B |
| NGO2174 | SEQ ID Nos. 139 to 141 in ANNEX B |
| NGO2164 | SEQ ID Nos. 133 to 135 in ANNEX B |
| NGO1676 | SEQ ID Nos. 88 to 90 in ANNEX B |
| NGO1679 | SEQ ID Nos. 94 to 96 in ANNEX B |
| NGO1658 | SEQ ID Nos. 79 to 81 in ANNEX B |
| NGO1440 | SEQ ID Nos. 76 to 78 in ANNEX B |
| NGO0174 | SEQ ID Nos. 46 to 48 in ANNEX B |
| NGO0173 | SEQ ID Nos. 43 to 45 in ANNEX B |
| NGO0592 | SEQ ID Nos. 52 to 54 in ANNEX B |
| NGO1680 | SEQ ID Nos. 31 to 33 in ANNEX B |
| NGO0620 | SEQ ID Nos. 67 to 69 in ANNEX B |
| NGO1659 | SEQ ID Nos. 82 to 84 in ANNEX B |
| NGO1291 | SEQ ID Nos. 73 to 75 in ANNEX B |
| NGO0648 | SEQ ID Nos. 70 to 72 in ANNEX B |
| NGO0593 | SEQ ID Nos. 55 to 57 in ANNEX B |
| NGO1804 | SEQ ID Nos. 97 to 99 in ANNEX B |
| NGO0618 | SEQ ID Nos. 61 to 63 in ANNEX B |
| NGO0619 | SEQ ID Nos. 64 to 66 in ANNEX B |
| NGO1812 | SEQ ID NOs.28 to 30 in ANNEX B |
| NGO1890 | SEQ ID Nos. 121 to 123 in ANNEX B |
| NGO2098 | SEQ ID Nos. 127 to 129 in ANNEX B |
| NGO2100 | SEQ ID Nos. 130 to 132 in ANNEX B |
| A9Y61_RS02445 or NGO_t12 | SEQ ID Nos. 145 to 147 in ANNEX B |
| A9Y61_RS04515 or NGO_t15 | SEQ ID Nos. 151 to 153 in ANNEX B |
| A9Y61_RS04510 or NGO_t14 | SEQ ID Nos. 148 to 150 in ANNEX B |
| A9Y61_RS09170 or NGO_t37 | SEQ ID Nos. 228 to 230 in ANNEX B |
| A9Y61_RS00075 or NGO_t01 | SEQ ID Nos. 142 to 144 in ANNEX B |

In some embodiments, markers according to the instant disclosure can have a sequence identity of at least 80%, or 90%, up to 100% of the markers listed in Table 3. In particular, markers of the instant disclosure can have sequence identity of 93%, 94%, 95%, 96%, 97%, 98%, or 99% of the sequences indicated in Table 3.

The term “sequence identity” refers to a quantitative measurement of the identity between sequences of a polypeptide or a polynucleotide and, in particular, indicates the amount of characters that match between two different sequences. Commonly used similarity searching programs, such as BLAST, PSI-BLAST [10] [11] [12] [13], SSEARCH [14] [15] FASTA[16] and the HMMER3 9 [17] can produce accurate statistical estimates, ensuring that protein sequences that share significant similarity also have similar structures.

The identity between sequences is typically measured by a process that comprises the steps of aligning the two polypeptide or polynucleotide sequences to form aligned sequences, then detecting the number of matched characters, i.e. characters identical between the two aligned sequences, and calculating the total number of matched characters divided by the total number of aligned characters in each polypeptide or polynucleotide sequence, including gaps. The identity result is expressed as a percentage of identity.

Biomarker’s features of the RNA markers of Table 3, such as resistant CT ratios and values, susceptible CT ratio values, abundance and threshold values, are further illustrated in

TABLE 4

List of exemplary marker genes differentially expressed between an untreated sample and a sample treated with antibiotics
Table 4: List of exemplary marker genes differentially expressed between an untreated sample and a sample treated with antibiotics

| Locus Tag | Average Susceptible C:T ratio after 15 min exposure | Standard Deviation of Susceptible C:T ratio after 15 min exposure | Average Susceptible Relative Abundance (TPM) | Average Resistant C:T ratio after 15 min exposure | Standard Deviation of Resistant C:T ratio after 15 min exposure |
|---|---|---|---|---|---|
| NGO0340 | 2.757 | 0.811 | 886.752 | 0.917 | 0.014 |
| NGO1837 | 2.08 | 0.382 | 542.158 | 0.906 | 0.058 |
| NGO1843 | 2.053 | 0.089 | 596.162 | 0.856 | 0.036 |
| NGO2024 | 1.93 | 0.138 | 504.857 | 0.921 | 0.023 |
| NGO1845 | 1.981 | 0.083 | 793.435 | 0.872 | 0.03 |
| NGO1677 | 2.991 | 0.631 | 500.919 | 0.983 | 0.02 |
| NGO1844 | 2.084 | 0.051 | 719.419 | 0.815 | 0.034 |
| NGO0171 | 2.425 | 0.448 | 425.961 | 0.913 | 0.02 |
| NGO1834 | 2.161 | 0.123 | 418.234 | 0.892 | 0.055 |
| NGO0172 | 3.551 | 1.865 | 273.08 | 0.892 | 0.031 |
| NGO1835 | 2.149 | 0.21 | 507.389 | 0.891 | 0.061 |
| NGO1673 | 4.902 | 2.876 | 227.951 | 0.931 | 0.005 |
| NGO1833 | 2.2 | 0.146 | 441.363 | 0.879 | 0.074 |
| NGO2173 | 2.629 | 0.25 | 429.176 | 0.962 | 0.019 |
| NGO0604 | 2.452 | 0.162 | 504.385 | 0.881 | 0.037 |
| NGO0016 | 3.348 | 0.64 | 161.719 | 0.946 | 0.034 |
| NGO2174 | 2.8 | 0.264 | 422.688 | 0.955 | 0.046 |
| NGO2164 | 2.978 | 0.257 | 136.319 | 0.901 | 0.043 |
| NGO1676 | 3.237 | 0.371 | 626.49 | 0.936 | 0.006 |
| NGO1679 | 5.201 | 2.268 | 317.278 | 0.952 | 0.074 |
| NGO1658 | 3.428 | 0.841 | 127.162 | 0.957 | 0.058 |
| NGO1440 | 4.781 | 1.963 | 140.148 | 0.916 | 0.065 |
| NGO0174 | 4.187 | 1.653 | 368.372 | 0.885 | 0.04 |
| NGO0173 | 4.834 | 2.216 | 400.084 | 0.967 | 0.054 |
| NGO0592 | 6.014 | 1.977 | 145.561 | 0.92 | 0.062 |
| NGO1680 | 5.579 | 1.488 | 522.393 | 0.931 | 0.052 |
| NGO0620 | 4.147 | 0.727 | 61.968 | 0.937 | 0.091 |
| NGO1659 | 5.667 | 1.791 | 73.859 | 0.939 | 0.076 |
| NGO1291 | 5.105 | 1.164 | 77.971 | 0.998 | 0.061 |
| NGO0648 | 4.959 | 1.74 | 57.357 | 1.115 | 0.169 |
| NGO0593 | 4.643 | 0.768 | 70.256 | 0.951 | 0.018 |
| NGO1804 | 5.062 | 1.772 | 103.085 | 0.901 | 0.09 |
| NGO0618 | 4.323 | 0.445 | 91.615 | 0.874 | 0.063 |
| NGO0619 | 5.758 | 1.431 | 63.787 | 0.88 | 0.054 |
| NGO1812 | 4.875 | 0.352 | 1142.564 | 0.897 | 0.027 |
| NGO1890 | 9.946 | 6.62 | 55.955 | 0.829 | 0.022 |
| NGO2098 | 7.087 | 2.034 | 30.66 | 0.927 | 0.072 |
| NGO2100 | 6.593 | 0.696 | 24.365 | 0.816 | 0.029 |
| A9Y61_RS02445 or NGO_t12 | 6.495 | 2.273 | 72.289 | 1.113 | 0.114 |
| A9Y61_RS04515 or NGO_t15 | 3.064 | 0.94 | 561.339 | 1.343 | 0.109 |
| A9Y61_RS04510 or NGO_t14 | 2.9 | 0.827 | 889.592 | 1.119 | 0.048 |
| A9Y61_RS09170 or NGO_t37 | 3.396 | 1.001 | 128.355 | 1.006 | 0.165 |
| A9Y61_RS00075 or NGO_t01 | 4.086 | 0.448 | 64.178 | 1.046 | 0.248 |

In the illustration of Table 4, for each marker, the range of possible threshold C:T ratios is calculated as a range between the mean Cr:Tr ratios for resistant and the mean Cs:Ts ratios for susceptible isolates, and narrowed down further to account for variability of the Cr:Tr ratios for resistant and the Cs:Ts ratios of susceptible isolates.

In some embodiments, after the marker is selected, when testing a sample with bacteria of unknown susceptibility to an antibiotic, the C:T ratio for this marker obtained from this sample is compared with Cs:Ts and Cr:Tr ratios. In some embodiments the C:T ratio thus obtained can be assigned as belonging to susceptible or resistant organism based on a threshold value.

For example, for a marker downregulated in the susceptible bacteria, the Cr:Tr values will be smaller than Cs:Ts values and a threshold value can be set above Cr:Tr value(s) and below Cs:Ts value(s). If a detected C:T is below threshold, we call it resistant and if CT is above threshold we call susceptible. In particular the threshold value can be set based on the knowledge of a distribution of a parameter indicative of the expression of one or more transcripts, to include transcripts differentially expressed in treated vs control sample across the distribution. In particular the threshold value for a C:T ratio can be set based on the knowledge of Cs:Ts and Cr:Tr distributions of a given transcript. In some embodiments, the threshold value is set at the average between the means of Cs:Ts and Cr:Tr distributions. In some embodiments, especially when the Cs:Ts and Cr:Tr distributions have unequal variance, the threshold value is set to between the means of Cs:Ts and Cr:Tr distributions at a value where the overlap between Cs:Ts and Cr:Tr distributions is zero or minimized.

In some embodiments, the threshold value can be selected among any one of the value within the following ranges 0.931-1.946, 0.964-1.698, 0.892-1.964., 0.944-1.792, 0.902-1.898, 1.003-2.360, 0.849-2.033, 0.933-1.977, 0.947-2.038., 0.923-1.686, 0.952-1.939, 0.936-2.026, 0.953-2.054, 0.981-2.379, 0.918-2.290, 0.980-2.708, 1.001-2.536, 0.944-2.721, 0.942-2.866, 1.026-2.933, 1.015-2.587, 0.981-2.818, 0.925-2.534, 1.021-2.618, 0.982-4.037, 0.983-4.091, 1.028-3.420, 1.015-3.876, 1.059-3.941, 1.284-3.219, 0.969-3.875, 0.991-3.290, 0.937-3.878, 0.934-4.327, 0.924-4.523, 0.851-3.326, 0.999-5.053, 0.845-5.897, 1.227-4.222 as will be understood by a skilled person upon reading of the present disclosure.

In some embodiments the RNA markers of N. gonorrhoeae herein described can have the following sequences indicated properties indicated in Table 5.

TABLE 5

List of exemplary marker genes differentially expressed between an untreated sample and a sample treated with antibiotics.

| | |
|---|---|
| Locus Tag | Description of putative or verified functionality associated to the marker |
| NGO0340 | cysteine synthase A (cysK) |
| NGO1837 | 50S ribosomal protein L4 (rplD) |
| NGO1843 | elongation factor G (fusA) |
| NGO2024 | 50S ribosomal protein L13 (rplM) |
| NGO1845 | 30S ribosomal protein S12 (rpsL) |
| NGO1677 | 50S ribosomal protein L27 (rpmA) |
| NGO1844 | 30S ribosomal protein S7 |
| NGO0171 | 50S ribosomal protein L19 (rplS) |
| NGO1834 | 30S ribosomal protein S19 (rpsS) |
| NGO0172 | tRNA (guanine-N(1)-)-methyltransferase (trmD) |
| NGO1835 | 50S ribosomal protein L2 (rplB) |
| NGO1673 | type IV pilus assembly protein (pilB) |
| NGO1833 | 50S ribosomal protein L22 (rplV) |
| NGO2173 | 50S ribosomal protein L32 (rpmF) |
| NGO0604 | 30S ribosomal protein S1 (rpsA) |
| NGO0016 | preprotein translocase subunit (secG) |
| NGO2174 | hypothetical protein |
| NGO2164 | GMP synthase (guaA) |
| NGO1676 | 50S ribosomal protein L21 (rplU) |
| NGO1679 | 50S ribosomal protein L33 (rpmG) |
| NGO1658 | hypothetical protein |
| NGO1440 | macrolide transport protein MacA |
| NGO0174 | 30S ribosomal protein S16 (rpsP) |
| NGO0173 | ribosome maturation factor RimM (rimM) |
| NGO0592 | trigger factor (tig) |
| NGO1680 | 50S ribosomal protein L28 (rpmB) |
| NGO0620 | aspartate alpha-decarboxylase |
| NGO1659 | intracellular septation protein A |
| NGO1291 | transcriptional regulator (yebC) |
| NGO0648 | membrane protein |
| NGO0593 | ATP-dependent Clp protease proteolytic subunit (clpP) |
| NGO1804 | (3R)-hydroxymyristoyl-ACP dehydratase (fabZ) |
| NGO0618 | membrane protein |
| NGO0619 | 2-dehydro-3-deoxyphosphooctonate aldolase |
| NGO1812 | major outer membrane protein (porB) |
| NGO1890 | glutamate permease; sodium/glutamate symport carrier protein |
| NGO2098 | diaminopimelate decarboxylase |
| NGO2100 | frataxin-like protein (cyaY) |
| A9Y61_RS02445 or NGO_t12 | tRNA-Serine |
| A9Y61_RS04515 or NGO_t15 | tRNA-Serine |
| A9Y61 _RS04510 or NGO_t14 | tRNA-Leucine |
| A9Y61_RS09170 or NGO_t37 | tRNA-Arginine |
| A9Y61 _RS00075 or NGO_t01 | tRNA-Leucine |

In preferred embodiments, the transcript can comprise at least one of a transcript of N. gonorrhoeae gene having locus tag NGO1812, a transcript of N. gonorrhoeae gene having locus tag NGO1680), a transcript of N. gonorrhoeae gene having locus tag NGO1291, a transcript of N. gonorrhoeae gene having locus tag NGO1673, a transcript of a transcript of N. gonorrhoeae gene having locus tag NGO0592 and a transcript of N. gonorrhoeae gene having locus tag NGO0340.

In more preferred embodiments, the transcript comprises or is at least one of a transcript N. gonorrhoeae gene having locus tag NGO1812 and possibly and putatively encoding major outer membrane protein (porB), and N. gonorrhoeae gene having locus tag NGO1680 and possibly and putatively encoding 50S ribosomal protein L28 (rpmB).

In some embodiments of the instant disclosure a method is described to detect in an N. gonorrhoeae bacteria, a N. gonorrhoeae transcript, which comprises
quantitatively detecting a transcript expression value of an RNA marker of N. gonorrhoeae selected from anyone of the RNA markers of N. gonorrhoeae herein described, in the N. gonorrhoeae following and/or upon contacting of the N. gonorrhoeae with an antibiotic to obtain an antibiotic treated transcript expression value for the RNA marker of .

In some embodiments, the method further comprises detecting whether there is a downshift in the transcript expression value of the RNA marker of N. gonorrhoeae following and/or upon the contacting of the N. gonorrhoeae with the antibiotic by comparing the antibiotic treated transcript expression value with an untreated marker expression valuean untreated marker expression value indicating of the expression of the RNA marker of N. gonorrhoeae in N. gonorrhoeae in absence of antibiotic treatment.

In some embodiments, the reference expression value of the RNA marker of N. gonorrhoeae in absence of antibiotic treatment is a control transcript expression value obtained by quantitatively detecting the RNA of N. gonorrhoeae in a control sample not treated with the antibiotic. In some embodiments, the reference transcript expression value of the RNA marker of N. gonorrhoeae is a transcript expression value obtained by quantitatively detecting the RNA of N. gonorrhoeae in the same sample prior to treatment with the antibiotic. In some embodiments, the reference transcript expression value of the RNA marker of N. gonorrhoeae is a transcript expression value obtained by quantitatively detecting the RNA of N. gonorrhoeae at time zero of the RNA expression of the transcript.

Accordingly, in some embodiments, the method to detect in an N. gonorrhoeae bacteria an N. gonorrhoeae transcripts can be performed by
contacting a sample of an isolate or specimen comprising the N. gonorrhoeae with an antibiotic to obtain an antibiotic treated sample,
quantitatively detecting a transcript expression value of a RNA marker of N. gonorrhoeae herein described in the antibiotic treated sample at one or more times following and/or upon contacting the sample with the antibiotic, to provide an antibiotic treated transcript expression value for the RNA marker of N. gonorrhoeae; and
detecting whether there is a downshift of the quantitatively detected transcript of the RNA marker of N. gonorrhoeae herein described in the treated sample with respect to an untreated marker expression valuean untreated marker expression value indicative of the expression of the RNA marker of N. gonorrhoeae in N. gonorrhoeae in absence of antibiotic treatment.

In some embodiments an untreated marker expression value indicative of the expression of the RNA marker of N. gonorrhoeae in N. gonorrhoeae in absence of antibiotic treatment is a control transcript expression value obtained by
quantitatively detecting a transcript expression value of the RNA marker of N. gonorrhoeae herein described in a control sample of the isolate or specimen comprising the N. gonorrhoeae, to provide a control transcript expression value of the RNA marker of N. gonorrhoeae herein described.

In some embodiments, the RNA markers of N. gonorrhoeae herein described can be used in a method to perform an antibiotic susceptibility test for N. gonorrhoeae. The method comprises detecting susceptibility to an antibiotic of an N. gonorrhoeae, by quantitatively detecting in a sample comprising the N. gonorrhoeae a transcript expression value of an RNA marker of N. gonorrhoeae selected from the RNA markers of an N. gonorrhoeae herein described following and/or upon contacting the sample with the antibiotic.

In the method to perform an antibiotic susceptibility test for N. gonorrhoeae the quantitatively detecting is performed to obtain an antibiotic treated transcript expression value for the RNA marker of N. gonorrhoeae suitable to detect susceptibility to the antibiotic of the N. gonorrhoeae in the sample.

In some embodiments, the method to perform an antibiotic susceptibility test for N. gonorrhoeae further comprises detecting whether there is a downshift of the transcript expression value with respect to the expression of the transcript in an untreated sample of the same specimen by comparing the detected antibiotic transcript expression value with an untreated marker expression value indicative of the transcript expression in the sample in absence of antibiotic treatment.

In some embodiments, the RNA markers of N. gonorrhoeae herein described can be used in a method to detect an RNA marker of susceptibility to an antibiotic in N. gonorrhoeae in a sample comprising the N. gonorrhoeae. The method comprises contacting the sample with the antibiotic to obtain an antibiotic treated sample and quantitatively detecting in the antibiotic treated sample one or more of the RNA markers of N. gonorrhoeae herein described.

In some embodiments, the method to detect an RNA marker of susceptibility to an antibiotic in N. gonorrhoeae further comprises detecting a downshift of an RNA marker selected from any one of the transcripts of N. gonorrhoeae genes herein described with respect to an untreated marker expression value indicative of the expression of the RNA marker of N. gonorrhoeae in N. gonorrhoeae in absence of antibiotic treatment.

The RNA markers of N. gonorrhoeae herein described can be used in a method to diagnose susceptibility to an antibiotic of a N. gonorrhoeae infection in an individual. The method comprises contacting a sample from the individual with the antibiotic; and quantitatively detecting expression by the N. gonorrhoeae in the sample of a marker of antibiotic susceptibility in N. gonorrhoeae selected from any one of the transcripts of N. gonorrhoeae genes herein described. In the method, the quantitatively detecting is performed following contacting the sample with the antibiotic. The method further comprises detecting whether there is a downshift of the detected transcript presence in the antibiotic sample with respect to an untreated marker expression value indicative of the expression of the marker of antibiotic susceptibility in N. gonorrhoeae to diagnose the antibiotic susceptibility of the N. gonorrhoeae infection in the individual.

The RNA markers of N. gonorrhoeae herein described can be used in a method to detect antibiotic susceptibility of an N. gonorrhoeae bacterium and treat N. gonorrhoeae in an individual. The method comprises contacting a sample from the individual with an antibiotic, and quantitatively detecting in the sample expression by the N. gonorrhoeae bacteria of a marker of antibiotic susceptibility selected from any one of the transcripts of N. gonorrhoeae genes herein described. In the method, the quantitatively detecting is performed following and/or upon contacting the sample with the antibiotic.

The method further comprises diagnosing antibiotic susceptibility of N. gonorrhoeae infection in the individual when a downshift in expression of at least one of the detected markers in the sample is detected in comparison with an untreated marker expression value indicative of the expression of the at least one of the detected markers in the sample from the individual in absence of antibiotic treatment.

The method also comprises administering an effective amount of the antibiotic to the diagnosed individual.

The term “individual” as used herein in the context of treatment includes a single biological organism, including but not limited to, animals and in particular higher animals and in particular vertebrates such as mammals and in particular human beings

In embodiments of the methods of the instant disclosure using any one of the N. gonorrhoeae markers herein described, contacting the N. gonorrhea can be performed by adding antibiotics to the microorganism and incubating the sample under certain condition

In particular in some embodiments, the antibiotic for treating the sample herein described can be provided in a sample comprising N. gonorrhoeae at a concentration equal to or the breakpoint MIC for the N. gonorrhoeae, to the antibiotic. In particular, the antibiotic for treating the sample herein described can be provided at a concentration lower than the breakpoint MIC for the N. gonorrhoeae strain in the sample, for example 1.5 times (or 1.5X) lower, 2 times (or 2X) lower, 3 times (or 3X) lower, 4 times (or 4X) lower, 8 times (or 8X) lower, or 16 times (or 16X) lower than the breakpoint MIC for a resistant isolate.. In some embodiments, the antibiotic for treating the sample herein described can be provided at a concentration higher than the breakpoint MIC for the N. gonorrhoeae strain in the sample, for example 1.5 times (or 1.5X) higher, 2 times (or 2X) higher, 3 times (or 3X) higher, or 4 times (or 4X) higher, or 8 times higher (8X) or 16 times higher (or 16X) than the breakpoint MIC for a resistant isolate. The breakpoint MIC of the antibiotic for the N. gonorrhoeae strain in the sample, can be obtained from the Clinical & Laboratory Standards Institute (CLSI) guidelines, European Committee of Antimicrobial Susceptibility Testing (EUCAST) or other sources identifiable to a skilled person.

In some embodiments, samples may be treated at several concentrations of the antibiotic to measure MIC of an organism and/or to determine if a sample contains bacteria with intermediate susceptibility, susceptible bacteria, or resistant bacteria to the antibiotic of interest. In order to determine the MIC using the described method, samples can be treated at multiple concentrations of antibiotic. These concentrations would include multiple dilutions below the susceptible MIC breakpoint, dilutions between the susceptible and resistant MIC breakpoints (including intermediate breakpoint concentrations), as well as a dilution above the resistant MIC breakpoint (see Example 13) To determine, degree of susceptibility, the sample would be exposed to three concentrations of antibiotic: a concentration equal to the susceptible MIC breakpoint, a concentration equal to the concentration of the resistant MIC breakpoint, and a concentration equal to the average of the maximum and minimum of the intermediate MIC breakpoint range. Susceptibility would then be determined , for example, by measuring the slope obtained by fitting a curve or line to the three points on the C:T ratio vs treatment concentration plot, and/or by comparing the relative difference in C:T ratio between the low and intermediate concentration of antibiotic and the difference in CT ratio between the intermediate and high concentration, and/or by comparing the magnitude of the value relative to a pre-defined threshold, or a combination of these analyses (see Example 14).

In some embodiments of the methods of the instant disclosure using any one of the N. gonorrhoeae markers herein described, the time period of contacting the sample with an antibiotic can be up to 5 minutes, 10 minutes, 15 minutes, 20 minutes, 25 minutes, 30 minutes up to 60 up to 90 up to 120 or higher, inclusive of any value therebetween or fraction thereof.

In some embodiments of the methods of the instant disclosure using any one of the N. gonorrhoeae markers herein described, the time period of contacting the sample with an antibiotic is shorter than the doubling time of the N. gonorrhoeae strain in the sample. For example, for conditions with N. gonorrhoeae doubling time of 45 minutes, 1 hour, 1.5 hours, or 2 hours, antibiotic exposure contacting time could be less than the time indicated in Table 6 below

TABLE 6

time of contacting N. gonorrhoeae with antibiotic

| | 45 minute doubling | 60 min doubling | 90 min doubling | 120 doubling |
|---|---|---|---|---|
| factor X | | contacting time, less than (minutes): | | |
| 1 | 45 | 60 | 90 | 120 |
| 0.75 | 33.75 | 45 | 67.5 | 90 |
| 0.5 | 22.5 | 30 | 45 | 60 |
| 0.35 | 15.75 | 21 | 31.5 | 42 |
| 0.25 | 11.25 | 15 | 22.5 | 30 |
| 0.2 | 9 | 12 | 18 | 24 |
| 0.15 | 6.75 | 9 | 13.5 | 18 |
| 0.1 | 4.5 | 6 | 9 | 12 |
| 0.075 | 3.375 | 4.5 | 6.75 | 9 |
| 0.05 | 2.25 | 3 | 4.5 | 6 |

In methods of the instant disclosure using any one of the N. gonorrhoeae markers herein described, incubation of a sample with an antibiotic can be performed at a temperature such that a physiological response to the antibiotic is generated in N. gonorrhoeae. The contacting is performed typically in an incubation temperature at 37° C., in an incubation temperature within the range of 36-38° °C, in an incubation temperature within the range of 35-39° °C.

In methods of the instant disclosure using any one of the N. gonorrhoeae markers herein described, the contacting can be performed by adding antibiotics to the microorganism and incubating the sample under certain condition preferably following and/or upon contacting the sample with a treatment media designed to support physiological processes of N. gonorrhoeae, enable or accelerate DNA replication and translation, maintain cellular uniformity and homogeneity in suspension, and promote interaction of the N. gonorrhoeae and antibiotic herein described.

In methods of the instant disclosure using any one of the N. gonorrhoeae markers herein described, quantitatively detecting an antibiotic treated transcript expression value in the treated sample can be performed following and/or upon contacting the sample with an antibiotic for a time period up to 20 minutes.

In some embodiments of the methods of the instant disclosure using any one of the N. gonorrhoeae markers herein described, quantitatively detecting transcript expression value can be performed by RNA-seq, qPCR, digital PCR, isothermal techniques such as LAMP, digital isothermal amplification methods, or using probes specifically targeting any one of the differentially expressed transcripts herein described. Additional techniques include microarrays and nanostring^{tm} as will be understood by a person skilled in the art.

In some embodiments, detecting specific gene expression can be performed at the transcription level by performing RNA sequencing (RNA-seq) and calculating RNA expression values based on the sequence data.

In some embodiments, the RNA expression values can be calculated as transcripts per million (TPM) as will be understood by a person skilled in the art. To calculate TPM, read counts are first divided by the length of each gene in kilobases, which gives reads per kilobase (RPK). RPKs for all genes are added and the sum is divided by 1,000,000. This gives the “per million” scaling factor. Finally, the RPK value for each genes is divided by the “per million” scaling factor to give TPM. [3]

In some embodiments of the methods of the instant disclosure using any one of the N. gonorrhoeae markers herein described, quantitatively detecting a treated gene expression pattern of the transcript can be performed using probes specifically targeting any one of the differentially expressed transcripts herein described.

The term “probe” as described herein indicates a molecule or computer support tool capable of specifically detect a target molecule such as one of the markers herein described. The wording “specific” “specifically” or “specificity” as used herein with reference to the binding of a first molecule to second molecule refers to the recognition, contact and formation of a stable complex between the first molecule and the second molecule, together with substantially less to no recognition, contact and formation of a stable complex between each of the first molecule and the second molecule with other molecules that may be present. Exemplary specific bindings are antibody-antigen interaction, cellular receptor-ligand interactions, polynucleotide hybridization, enzyme substrate interactions and additional interactions identifiable by a skilled person. The wording “specific” “specifically” or “specificity” as used herein with reference to a computer supported tool, such as a software indicates a tool capable of identifying a target sequence (such as the one of a marker herein described) among a group of sequences e.g. within a database following alignment of the target sequence with the sequences of the database. Exemplary software configured to specifically detect target sequences comprise Primer-3, PerlPrimer and PrimerBlast.

In methods of the instant disclosure using any one of the N. gonorrhoeae transcripts herein described, treatment of the N. gonorrhoeae bacteria with a probe and/or antibiotic or with any other reagents functional to perform the related step is performed on samples.

In some embodiments of the methods of the instant disclosure using any one of the N. gonorrhoeae markers herein described, the probe specific for the transcript is selected from a primer having a sequence specific for the marker, or an antibody specific for the marker.

In particular, probes usable in methods herein described can include primers for nucleic acid amplification reactions (such as PCR, LAMP, HAD, RPA, NASBA, RCA, SDA, NEAR, and additional reactions identifiable by a skilled person), including digital single molecule versions of these reactions and including real-time versions of these reactions, molecular beacons that include dyes, quenchers, or combinations of dyes and quenchers.

Nucleic acid probes preferably have sequences that complementarily bind to the DNA and/or RNA sequences of the markers described herein, and can be used to target RNA molecules directly, or DNA molecules that result, for example, from reverse transcription of the target RNA molecules (such molecules may be referred to as cDNA). In embodiments of the present disclosure when two polynucleotide strands, sequences or segments are noted to be binding to each other through complementarily binding or complementarily bind to each other, this indicate that a sufficient number of bases pairs forms between the two strands, sequences or segments to form a thermodynamically stable double-stranded duplex, although the duplex can contain mismatches, bulges and/or wobble base pairs as will be understood by a skilled person.

The term “thermodynamic stability” as used herein indicates a lowest energy state of a chemical system. Thermodynamic stability can be used in connection with description of two chemical entities (e.g. two molecules or portions thereof) to compare the relative energies of the chemical entities. For example, when a chemical entity is a polynucleotide, thermodynamic stability can be used in absolute terms to indicate a conformation that is at a lowest energy state, or in relative terms to describe conformations of the polynucleotide or portions thereof to identify the prevailing conformation as a result of the prevailing conformation being in a lower energy state. Thermodynamic stability can be detected using methods and techniques identifiable by a skilled person. For example, for polynucleotides thermodynamic stability can be determined based on measurement of melting temperature Tₘ, among other methods, wherein a higher Tₘ can be associated with a more thermodynamically stable chemical entity as will be understood by a skilled person. Contributors to thermodynamic stability can include, but are not limited to, chemical compositions, base compositions, neighboring chemical compositions, and geometry of the chemical entity.

In embodiments herein described, primer and/or other nucleic acid probes can be designed to complementarily bind the target marker herein described with methods described in [13].

Probes usable in methods herein described include probes used in guiding CRISPR-based detection of nucleic acids. e.g. CRISPR-associated protein-9 nuclease; CRISPR-associated nucleases. An example of a CRISPR-based method is described in references [18] [19] [20]. Such probes can be synthesized using naturally occurring nucleotides including deoxyInosine, or include unnatural nucleotides such as locked nucleic acid (LNA). Probes can comprise dyes, quenchers, or combinations of dyes and quenchers attached to the probe. Hybridization probes, including those used in fluorescent in situ hybridization and hybridization chain reaction. Probes can also comprise electrochemically active redox molecules attached to the probe. Probes can be provided in a dry state. Probes can also include probes bound to beads, such beads may be fluorescently labeled. Probes can also include probes bound to nanoparticles, such nanoparticles may include gold nanoparticles. Probes can include probes disposed in arrays of wells with volumes less than 50 microliters, and/or wells within plastic substrates. Exemplary probes suitable to be used in methods using any one of the N. gonorrhoeae markers herein described comprise probes provided with the commercially available technology such as the technology of any of the companies GenProbe, Nanosphere, Luminex, Biofire and additional companies identifiable by a skilled person.

In some embodiments, quantitative detection of the marker/transcript is performed by one or more methods including Northern blotting, Nuclease Protection Assays (NPAs) in situ hybridization, reverse transcription polymerase chain reaction, and qPCR.

In some embodiments, of the methods of the instant disclosure using any one of the N. gonorrhoeae markers herein described, quantitatively detecting of a marker can be performed by detecting a detectable portion thereof. Exemplary detectable portions comprise to regions of at least 14 base pair, at least 16 base pair, at least 18 base pair, at least 19 base pair, at least 20 base pair, at least 21 base pair, at least 22 base pair, at least 23 base pair, at least 24 base pair, at least 30 base pair, at least 40 base pair, at least 50 base pair, at least 60 base pair, at least 70 base pair, at least 80 base pair, at least 90 base pair, or at least 100 base pair, The specific portion can be identified by a skilled person based on the length of the transcript to be detected as will be understood by a skilled person.

In some embodiments of the methods of the instant disclosure using any one of the N. gonorrhoeae markers herein described, quantitatively detecting individual tRNA markers can be performed with quantification methods comparable with method used for detection of other RNA markers above. The secondary structure and multitude of base modifications prevalent on tRNA often makes reverse transcription inefficient and thus a variety of modified reverse transcription steps can be used. These can involve more flexible reverse transcriptases (RTs) like group II intron reverse transcriptase[21] [22].

In some embodiments of the methods of the instant disclosure using any one of the N. gonorrhoeae markers herein described, the methods comprise detecting whether there is a shift in the transcript expression of the markers, in a sample treated with an antibiotic with respect to a sample not treated with antibiotic.

In particular, in embodiments of the methods of the instant disclosure using any one of the N. gonorrhoeae markers herein described, the methods comprise detecting whether there is a downshift of a detected presence in N. gonorrhoeae of a N. gonorrhoeae marker following treatment with antibiotic with respect to an untreated marker expression value indicative of the expression in N. gonorrhoeae of the one or more N. gonorrhoeae marker in absence of antibiotic treatment.

In some embodiments of the methods of the instant disclosure using any one of the N. gonorrhoeae markers, the reference expression value is a control transcript expression value of the RNA marker of N. gonorrhoeae detected in a control sample of the specimen, and detecting whether there is a downshift can be performed by comparing the antibiotic treated transcript expression value with respect to the control transcript expression value of the RNA marker of N. gonorrhoeae in a control sample of the specimen.

Therefore, in some embodiments of the methods of the instant disclosure using any one of the N. gonorrhoeae markers, the reference expression value indicative of the expression of the RNA marker of N. gonorrhoeae in absence of antibiotic treatment is a control transcript expression value obtained by quantitatively detecting the RNA of N. gonorrhoeae marker in a control sample not treated with the antibiotic.

A shift in the expression of the markers can be determined by calculating differential gene expression levels (C:T ratios) as described above in connection with methods to identify a marker of antibiotic susceptibility.

In particular in methods of the instant disclosure using any one of the N. gonorrhoeae markers, the methods can comprise for a specimen comprising N. gonorrhoeae (e.g. from an individual).
providing a treated N. gonorrhoeae sample treated with the antibiotic and a control N. gonorrhoeae sample not treated with the antibiotic,
quantitatively detecting a control N. gonorrhoeae gene expression value C for a N. gonorrhoeae marker gene in the control N. gonorrhoeae sample,
quantitatively detecting a treated N. gonorrhoeae gene expression value T for the N. gonorrhoeae marker gene in the treated N. gonorrhoeae sample, and
providing a N. gonorrhoeae (C:T) value for the N. gonorrhoeae marker gene by dividing C for the N. gonorrhoeae marker gene by T for the N. gonorrhoeae marker gene detected in the sample, and
detecting differential expression of the N. gonorrhoeae marker gene based on the N. gonorrhoeae C:T value:

In some embodiments of the methods of the instant disclosure using any one of the N. gonorrhoeae markers herein described, the marker comprises more than one marker.

In some embodiments of the methods of the instant disclosure using any one of the N. gonorrhoeae markers herein described, the N. gonorrhoeae bacteria is selected from any strain of N. gonorrhoeae including its genetic variants.

In some embodiments, the C:T ratio can be provided by RPKM (reads per kilobase per million mapped reads). The use of RPKM and comparison to TPM is described for example in Wagner et al 2012 [3]. In some embodiments the C:T ratio is provided by FPKM (fragments per kilobase per million), the use of FPKM is described for example in Conesa et al. 2016 [4]. These units normalize for sequencing depth and transcript length. In some embodiments RPM (reads per million mapped reads; RPM does not normalize for transcript length) or raw sequencing read counts can be used. The related methods are identifiable by a skilled person upon reading of the present disclosure.

In methods of the instant disclosure using any one of the N. gonorrhoeae markers, the differential expression of the N. gonorrhoeae marker can be expressed in accordance with a fold change approach in view of the C:T ratios identifiable by a skilled person upon reading of the present disclosure. In particular in the fold-change approach, a gene is considered to be differentially expressed if the ratio of the marker expression level between the antibiotic treated and untreated conditions exceeds a certain threshold, for example, 1.5-fold, twofold or threefold, or 4-fold or 5-fold change.

Accordingly, in some embodiments of the methods of the instant disclosure using any one of the N. gonorrhoeae markers at least 1.2-fold magnitude of fold change is considered as a shift. In some embodiments, contacting the sample with an antibiotic results the markers a 1.5 fold change or 2-fold or 4-fold change up to 6-fold change within the first 5 minutes of contact. Increasing the antibiotic exposure time can further shift the fold-change value.

In some embodiments of the methods of the instant disclosure using any one of the N. gonorrhoeae markers, the downshift of the transcript presence is at least 2-fold, 4-fold or is 6-fold or higher.

In preferred embodiments, the (C:T) value of an N. gonorrhoeae marker can be adjusted to reduce the impact of biological variability and/or technical variability in the C:T detection, more preferably of both biological and technical variability.

Accordingly, in some embodiments, any one of the methods of the instant disclosure using any one of the N. gonorrhoeae markers herein described can further comprise normalizing the antibiotic treated transcript expression value, the control transcript expression value and/or the related ratio, before detecting whether there is a downshift in antibiotic treated sample with respect to the untreated sample.

In particular, in some embodiments, at least one of the antibiotic treated transcript expression value and the control transcript expression value are normalized before providing a C:T ratio. In some embodiments, the C:T ratio of the antibiotic treated transcript expression value and the control transcript expression value is normalized using reference measurements.

The normalization can be performed by dividing the antibiotic treated transcript expression value, the control transcript expression value and/or the related ratio, by a reference measurement of RNA, DNA, cell number, number of samples, effective amount of sample used and/or a related ratio in a control and in a treated sample, according to approaches indicated for methods to identify markers of antibiotic susceptibility of the disclosure.

In particular, in some of these embodiments, the quantitatively detecting can be performed at a plurality of times following and/or upon contacting the sample, and/or under several conditions following and/or upon contacting the sample. For example in some of these embodiments, the antibiotic can be added at different concentrations. Also, in some of those embodiments adding the antibiotic can be performed in the treated N. gonorrhoeae sample throughout incubation or at set intervals during incubation to increase or decrease the physiological response of the N. gonorrhoeae to the antibiotic. Also in some of those embodiments, the quantitatively detecting can be performed at various times including time zero (for example, immediately prior or immediately after antibiotic treatment) of the transcript expression in the sample. In some of those embodiments, the quantitatively detecting can be performed at various temperatures and/or in multiple samples. In these embodiments, normalization can be performed by dividing the detected expression value and/or the related ratio between treated and control samples by the volume of samples or other reference measurements, such as the expression value of a reference RNA, level of DNA, cell numbers, as well as other reference parameters.

The control transcripts and related method of identification described in the method to identify markers of the present disclosure apply to the instant methods as will be understood by a skilled person.

Preferably, control transcripts are selected so this C:T ratio has low technical and biological variability, for example described by standard deviation with value of less than 0.5, less than 0.4, less than 0.3, less than 0.2, less than 0.1. In some embodiments, high-abundance transcripts (for example, transcripts in the top 10% of most expressed transcripts) are used to achieve low technical variability. Preferably, control transcripts are selected so this C:T ratio has low biological variability. Transcripts with high expression not affected by the antibiotic treatment are good candidates for control transcripts with low biological variability. For mRNA high expression level is obtained with more than 10 copies per cell or equivalent parameter in view of the method of measurement (for example RNAseq can have preferred expression levels for detection are TPM > 100 for any transcript and “high expression” being TPM > 100,000 (greater than 3000 copies/cell).

In some embodiments, a control transcript can be selected by providing a pool of isolates with known susceptibility; for each of these isolates, measuring a CT ratio of each transcript; and selecting as the control transcripts the transcripts with a CT ratio that is substantially the same in the pool of isolates between the susceptible isolates and the resistant isolates. The pool of isolates can be obtained from CDC Antimicrobial Resistance Isolate Bank. and/or from clinical collections of isolates.

Alternatively, the control transcript can be selected by measuring a CT ratio of each transcript in a strain subject to the antibiotic susceptibility test, i.e. with unknown susceptibility, and selecting as the control transcript the transcript with a CT ratio close to one, i.e. transcripts with expression not affected by the antibiotic treatment. Preferably, the control transcripts have a high expression level (e.g. with a TPM >10,000). Exemplary control transcripts comprise the transcript listed in Table 1.

In some embodiments, the control transcript can be a ribosomal RNA, including 23S rRNA, 16S rRNA, 5S rRNA and other RNA component of ribosome.

In some embodiments, 16S rRNA or 23 rRNA are used as reference RNA for normalization (see e.g. Table 2 of the instant disclosure).

In some embodiments of the fold-change approach, a gene is considered to be differentially expressed if the ratio of the normalized marker expression level between the antibiotic treated and untreated conditions exceeds a certain threshold, for example, 1.5 fold, twofold or threefold, or 4-fold or 5-fold change, wherein normalization can be performed with any of the methods herein described.

In some embodiments of any one of the methods of the instant disclosure using any one of the N. gonorrhoeae markers herein described, detecting whether there is a downshift can be performed by comparing the antibiotic treated transcript expression value of the RNA marker of N. gonorrhoeae with the expression value in the treated sample of a biomarker of the expression of the RNA marker of N. gonorrhoeae to detect the downshift. In particular, a biomarker of the expression can be any molecule and in particular a transcript, whose expression, under control conditions, has been previously shown to be correlated with the expression of the RNA marker of N. gonorrhoeae, preferably for a plurality of strains. In some embodiments, a downshift of expression of the RNA marker is detected when the ratio of expression of this marker to the expression of the biomarker of the expression in the treated sample is statistically significantly different than the range of ratios expected based on the analysis correlation of expression of these two markers under control conditions.

In some embodiments any one of the methods of the instant disclosure using any one of the N. gonorrhoeae markers herein described, the N. gonorrhoeae marker is a plurality of N. gonorrhoeae markers. In those embodiments the quantitative detection of the related expression can be performed by detecting global gene expression, or patterns of gene expression, in the tested samples for the plurality of the N. gonorrhoeae markers, as will be understood by a skilled person.

In methods of the instant disclosure using any one of the N. gonorrhoeae markers herein described, the sample can be provided from urine, swab, genital swab, throat swab, urethral swab, cervical swab, vaginal swab, oropharyngeal swab, throat swab, and rectal swabs. For urine sample, the preferable amount is between 1 ul and 10 ml. If the sample is provided as in swabs, the swab can be placed in an elution buffer to elute bacterial target cells from the swab. Samples can also include bacterial culture samples, for example, those grown on solid media such as chocolate agar, or grown in liquid culture such as Hardy Fastidious Broth (HFB).

In some embodiments of the methods of the instant disclosure using any one of the N. gonorrhoeae markers herein described, the sample can be pretreated to enrich RNA or a N. gonorrhoeae by removal of human RNA or RNA of other microorganisms. The removal of human RNA can be performed via hybridization to beads or columns with probes specific for human RNA. The removal of human RNA can also be performed via selective lysis of human cells and degradation of released human RNA. The sample may also be pretreated to enrich tRNA via size selection.

In general, in embodiments of the methods of the instant disclosure using any one of the N. gonorrhoeae markers herein described, enriching a sample can be performed with methods and approaches described for the methods to identify an antibiotic susceptibility marker of the disclosure.

In some embodiments of the methods of the instant disclosure using any one of the N. gonorrhoeae markers herein described, the sample can be stored until sample preparation and analysis, for example at room temperature, 4° C., -20° C., or -80° C., as appropriate, identifiable by those skilled in the art. When biological specimens are stored, ideally they remain equivalent to freshly-collected specimens for the purposes of analysis. In some embodiments, of the methods of the instant disclosure using any one of the N. gonorrhoeae markers herein described, the sample can be pre-incubated with growth media for a short period of time to increase the number of viable bacterial cells or to increase the level of RNA expression in such cells. The temperature and media for such pre-incubation can be performed as described herein for incubation. The duration of such pre-incubation can range, for example, from 5 minutes to 20 minutes to 1 hour to 2 hours.

In some embodiments of the instant disclosure wherein the microorganism is N. meningitidis, markers are expected to be selected from a transcript of a N. meningitidis gene based on the fact that Neisseria meningitidis also lacks the SOS response [23] ([24] (and [25] or a corresponding cDNA.

In particular, markers are expected to be selected from a transcript of a N. meningitidis gene comprise the ones listed in Table 7

TABLE 7

List of exemplary marker genes expected to be differentially expressed between an untreated sample and a sample treated with antibiotics

| | | | |
|---|---|---|---|
| Marker | DNA(+)strand | cDNA strand | RNA |
| PorB NC_003112.2:2157529-2158524 Neisseria meningitidis MC58 | SEQ ID NO:154 in ANNEX C | SEQ ID NO:155 in ANNEX C | SEQ ID NO:156 in ANNEX C |
| rpmB NC_003112.2:332567-332800 Neisseria meningitidis MC58 - on the (-)strand | SEQ ID NO:157 in ANNEX C | SEQ ID NO:158 in ANNEX C | SEQ ID NO:159 in ANNEX C |

In some embodiments, markers according to the instant disclosure can have a sequence identity of at least 80%, or 90%, up to 100% of the markers listed in Table 7. In particular markers of the instant disclosure can have sequence identity of 93%, 94%, 95%, 96%, 97%, 98%, or 99% of the sequences indicated in Table 7.

The RNA marker of N. meningitidis and/or corresponding cDNA can be used to detect a transcript of N. meningitidis., perform an antibiotic susceptibility test for N. meningitidis, detect an RNA marker of susceptibility to an antibiotic in N. meningitidis, diagnose susceptibility to an antibiotic of a N. meningitidis infection in an individual, and/or detect antibiotic susceptibility of an N. meningitidis bacterium and treat N. meningitidis in an individual, with methods and systems comprising the features indicated in any one of the third to the eighth aspect of the summary section and related portion of the detailed description of the instant disclosure in connection with N. gonorrhoeae transcripts and/or corresponding cDNA and their use in methods and systems related to the N. gonorrhoeae microorganism.

Methods of the present disclosure using any one of the N. gonorrhoeae transcripts and/or N. meningitidis herein described, can be performed with a corresponding system comprising at least one probe specific for a transcript herein described and/or or probe specific for cDNA a transcript herein described, and reagents for detecting the at least one probe. The at least one probe and reagents are included in the system for simultaneous combined or sequential use in any one of the methods of the present disclosure using any one of the N. gonorrhoeae transcripts herein described.

In particular, in the instant disclosure a system is described for performing at least one of the methods herein described to detect an N. gonorrhoeae transcript, to detect antibiotic susceptibility of N. gonorrhoeae bacteria, to perform an antibiotic susceptibility test for an N gonorrhoeae, and/or to diagnose and/or treat N. gonorrhoeae in an individual. The system comprises at least one probe specific for a transcript selected from any one of the transcripts of N. gonorrhoeae genes herein described, and/or a probe specific for cDNA a transcript herein described, and reagents for detecting the at least one probe.

In some embodiments of the system herein described the system comprises at least one probe specific for a transcript, and/or probe specific for a corresponding cDNA of said transcript, selected from at least one of a transcript of N. gonorrhoeae gene having locus tag NGO1812 and encoding major outer membrane protein (porB), a transcript of N. gonorrhoeae gene having locus tag NGO1680 and encoding 50S ribosomal protein L28 (rpmB), a transcript of N. gonorrhoeae gene having locus tag NGO1291 and encoding transcriptional regulator (yebC)a transcript of N. gonorrhoeae gene having locus tag NGO1673 and encoding type IV pilus assembly protein(pilB), a transcript of a transcript of N. gonorrhoeae gene having locus tag NGO0592 and encoding trigger factor (tig) and a transcript of N. gonorrhoeae gene having locus tag NGO0340 and encoding cysteine synthase A (cysK).

In some embodiments of the system herein described the system comprises at least one probe specific for a transcript and/or a corresponding cDNA, which comprises or is at least one of a transcript N. gonorrhoeae gene having locus tag NGO1812 and annotated as encoding major outer membrane protein (porB), and/or a corresponding cDNA and N. gonorrhoeae gene having locus tag NGO1680 and annotated as encoding 50S ribosomal protein L28 (rpmB) and/or a corresponding cDNA.

In some embodiments of the system herein described the system comprises primers configured to specifically hybridizes with the transcript and/or a corresponding cDNA. In some of these embodiments the system comprises a probe specific for a transcript of N. gonorrhoeae gene having locus tag NGO1812, the probe comprises a pair of primers having sequence GCTACGATTCTCCCGAATTTGCC (SEQ ID NO: 160) (CCGCCKACCAAACGGTGAAC (SEQ ID NO: 161), a probe specific for a transcript of N. gonorrhoeae gene having locus tag NGO1680 the probe comprises a pair of primers having sequence TTGCCCAACTTGCAATCACG (SEQ ID NO: 162) and AGCACGCAAATCAGCCAATAC (SEQ ID NO: 163). a probe specific for a transcript of N. gonorrhoeae gene having locus tag NGO1291 the probe comprises a pair of primers having sequence GCTTTGGAAAAAGCAGCCG (SEQ ID NO: 164) and GGTTTTGTTGTCGGTCAGGC (SEQ ID NO: 165), a probe specific for a transcript of N. gonorrhoeae gene having locus tag NGO1673, the probe comprises a pair of primers having sequence GACTTTTGCCGCTGCTTTG (SEQ ID NO: 166) and GCGCATTATTCGTGTGCAG (SEQ ID NO: 167), a probe specific for a transcript of N. gonorrhoeae gene having locus tag NGO0592 the probe comprises a pair of primers having sequence AAAGCCTTGGGTATTGCGG (SEQ ID NO: 168) and TGACCAAAGCAACCGGAAC (SEQ ID NO: 169). and/or a probe specific for a transcript of N. gonorrhoeae gene having locus tag NGO0340 the probe comprises a pair of primers having sequence GAGGCTTCCCCCGTATTGAG (SEQ ID NO: 170) and TTCAAAAGCCGCTTCGTTCG (SEQ ID NO: 171).

In some embodiments, the systems of the disclosure to be used in connection with methods herein described using any one of the N. gonorrhoeae transcripts herein described, the system further comprises a probe specific for a reference RNA and/or a corresponding cDNA. In some of these embodiments, the reference RNA is N. gonorrhoeae 16S rRNA the and the probe comprises a pair of primers having sequence the probe comprises a pair of primers having sequence ACTGCGTTCTGAACTGGGTG (SEQ ID NO: 172) and GGCGGTCAATTTCACGCG (SEQ ID NO: 173). In some of these embodiments, the control transcript is N. gonorrhoeae 23S rRNA and the probe comprises a pair of primers having sequence the probe comprises a pair of primers having sequence GCATCTAAGCGCGAAACTCG (SEQ ID NO: 174), and CCCCACCTATCAACGTCCTG (SEQ ID NO: 175).

In some embodiments, the systems of the disclosure to be used in connection with methods herein described using any one of the N. gonorrhoeae transcripts herein described or cDNA of any one of the N. gonorrhoeae transcripts herein described the system can further comprise an antibiotic formulated for administration to a sample in combination with the at least one probe.

In some embodiments, the systems of the disclosure to be used in connection with methods herein described using any one of the N. gonorrhoeae transcripts herein described and/or cDNA of any one of the N. gonorrhoeae transcripts herein described, the system further comprises an antibiotic formulated for administration to an individual in an effective amount to treat an N. gonorrhoeae infection in the individual.

In some embodiments, the systems of the disclosure to be used in connection with methods herein described using any one of the N. gonorrhoeae transcripts herein described, the reagents comprise RNA extraction kit and amplification mix. The system may also include one or more antibiotics and/or exposure media with or without the antibiotics. The system can also include reagents required for preparing the sample, such as one or more of buffers e.g. lysis, stabilization, binding, elution buffers for sample preparation, enzyme for removal of DNA e.g. DNase I, and solid phase extraction material for sample preparation., reagents required for quantitative detection such as intercalating dye, reverse-transcription enzyme, polymerase enzyme, nuclease enzyme (e.g. restriction enzymes; CRISPR-associated protein-9 nuclease; CRISPR-associated nucleases as described herein) and reaction buffer. Sample preparation materials and reagents may include reagents for preparation of RNA and DNA from samples, including commercially available reagents for example from Zymo Research, Qiagen or other sample preparations identifiable by a skilled person. The system can also include means for performing RNA quantification such as one or more of: container to define reaction volume, droplet generator for digital quantification, chip for digital detection, chip or device for multiplexed nucleic acid quantification or semiquantification, and optionally equipment for temperature control and detection, including optical detection, fluorescent detection, electrochemical detection.

In some embodiments, the system can comprise a device combining all aspects required for an antibiotic susceptibility test.

The systems herein disclosed can be provided in the form of kits of parts. In kit of parts for performing any one of the methods herein described, the probes and the reagents for the related detection can be included in the kit alone or in the presence of one or more antibiotic as well as any one of the RNA markers, corresponding cDNA and/or probes for one or more reference RNAs and/or corresponding cDNAs. In kit of parts for the treatment of an individual the probes and reagents for the related detection can be comprised together with the antibiotic formulated for administration to the individual as well as additional components identifiable by a skilled person.

In a kit of parts, the probes and the reagents for the related detection, antibiotics, RNA markers, and/or reference RNA and additional reagents identifiable by a skilled person are comprised in the kit independently possibly included in a composition together with suitable vehicle carrier or auxiliary agents. For example, one or more probes can be included in one or more compositions together with reagents for detection also in one or more suitable compositions.

Additional components can include labeled polynucleotides, labeled antibodies, labels, microfluidic chip, reference standards, and additional components identifiable by a skilled person upon reading of the present disclosure.

The terms “label” and “labeled molecule” as used herein refer to a molecule capable of detection, including but not limited to radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting fluorescence in a detectable image. As a consequence, the wording “labeling signal” as used herein indicates the signal emitted from the label that allows detection of the label, including but not limited to radioactivity, fluorescence, chemoluminescence, production of a compound in outcome of an enzymatic reaction and the like.

In embodiments herein described, the components of the kit can be provided, with suitable instructions and other necessary reagents, in order to perform the methods here disclosed. The kit will normally contain the compositions in separate containers. Instructions, for example written or audio instructions, on paper or electronic support such as tapes, CD-ROMs, flash drives, or by indication of a Uniform Resource Locator (URL), which contains a pdf copy of the instructions for carrying out the assay, will usually be included in the kit. The kit can also contain, depending on the particular method used, other packaged reagents and materials (i.e. wash buffers and the like).

Further details concerning the identification of the suitable carrier agent or auxiliary agent of the compositions, and generally manufacturing and packaging of the kit, can be identified by the person skilled in the art upon reading of the present disclosure.

### EXAMPLES

The methods and system herein disclosed are further illustrated in the following examples, which are provided by way of illustration and are not intended to be limiting.

### Example 1: Microorganisms’ Exposure to Antibiotic

Antibiotic susceptible and resistant clinical isolates were obtained from the University of California, Los Angeles, Clinical Microbiology Laboratory.

Isolates were plated from glycerol stocks onto Chocolate Agar plates and grown in static incubation overnight (37° C., 5% CO₂). Cells were re-suspended in Hardy Fastidious Broth (HFB) and incubated for 45 min (37° C., 5% CO₂) with shaking (800 rpm) to an OD₆₀₀ between 1 and 5. Cultures were diluted (5X) into HFB. Each isolate culture was split into “treated” and “control” tubes.

Ciprofloxacin was added to the “treated” tubes (final concentration of 0.5 µg/mL) and water was added to the “control” tubes; cultures were incubated (static; 37° C., 5% CO₂) for 15 min. During incubation, samples were collected for RNA sequencing at 5, 10, and 15 min (300 µL aliquot of sample was mixed into 600 µL of Qiagen RNA Protect Reagent (Qiagen, Hilden, Germany) for immediate RNA stabilization).

In addition, a sample was collected for RNA sequencing immediately before ciprofloxacin was added.

To quantify CFU, the sample at t = 15 min was serially diluted (10x), plated on a Chocolate Agar plate, and incubated overnight (37° C., 5% CO₂).

### Example 2: Microorganisms’ Exposure to Antibiotic

Antibiotic susceptible and resistant clinical isolates were obtained from the N. gonorrhoeae panel of the CDC Antimicrobial Resistance Isolate Bank. Isolates were plated from glycerol stocks onto Chocolate Agar plates and grown in static incubation overnight (37° C., 5% CO₂). Cells were re-suspended in pre-warmed HFB + 5 mM sodium bicarbonate and incubated for 30 min (37° C., 5% CO₂) with shaking (800 rpm) to an OD₆₀₀ between 1 and 5. Cultures were diluted (100X) into HFB + 5 mM sodium bicarbonate.

Each isolate culture was split into treated (0.5 µg/mL final concentration of ciprofloxacin) and control (water instead of antibiotic) samples. Samples were incubated at 37° C. for 10 min on a static hot plate. A 90 µLaliquot of each sample was placed into 180 µLof Qiagen RNA Protect Reagent for immediate RNA stabilization. A 5 µLaliquot of each sample was plated onto a Chocolate Agar plate and incubated overnight (37° C., 5% CO₂) as a control for the exposure experiments. If the expected growth phenotypes (i.e. resistant = growth; susceptible = no growth) were not observed for any single sample in the plating control, the exposure experiment was repeated for the set of samples.

From the 50 total isolates available from the N. gonorrhoeae panel of the CDC Antimicrobial Resistance Isolate Bank, 49 were used in this study. One isolate was excluded from this study because it is suspected that it had been contaminated; N. gonorrhoeae porB primer amplification was not detected using qPCR.

### Example 3: RNA Sequencing and Analysis

RNA was extracted using the Enzymatic Lysis of Bacteria protocol of the Qiagen RNeasy Mini Kit and processed according to the manufacturer’s protocol. DNA digestion was performed during extraction using the Qiagen RNase-Free DNase Set.

The quality of extracted RNA was measured using an Agilent 2200 TapeStation (Agilent, Santa Clara, CA, USA). Extracted RNA samples were prepared for sequencing using the NEBNext Ultra RNA Library Prep Kit for Illumina (New England Biolabs, Ipswitch, MA, USA) and the NEBNExt Multiplex Oligos for Illumina. Libraries were sequenced at 50 single base pair reads and a sequencing depth of 10 million reads on an Illumina HiSeq 2500 System (Illumina, San Diego, CA, USA) at the Millard and Muriel Jacobs Genetics and Genomics Laboratory, California Institute of Technology. Raw reads from the sequenced libraries were subjected to quality control to filter out low-quality reads and trim the adaptor sequences using Trimmomatic (version 0.35).

The reads were aligned to the FA 1090 strain of N. gonorrhoeae (NCBI Reference Sequence: NC_002946.2) using Bowtie2 (version 2.2.5) and quantified using the Subread package (version 1.5.0-p1). A pseudocount of 1 was added to the gene quantification; gene expression was defined in transcripts per million (TPM).

### Example 4: Marker Selection Based on C:T Ratio

For each gene, the C:T ratio was defined as the gene expression (TPM) in the control sample divided by the gene expression (in TPM) in the treated sample. The -log₂(C:T) was plotted against the -log₂(expression in TPM) for all genes. To identify genes that were differentially expressed between control and treated samples, a threshold of significance was defined.

The threshold of significance was calculated from the C:T ratios at t = 0 for the biological replicates that were sequenced (three susceptible and three resistant isolates). For each of the six gene expression datasets (one for each isolate), a negative exponential curve was fit to the outer edge of each plot and then the curves were averaged from all six datasets.

Finally, a 90% confidence interval was added to the average curve by assuming a Gaussian fit for the error distribution, which is the threshold of significance. Genes with a -log₂(C:T) value above or below the upper and lower thresholds were identified as differentially expressed. Genes that were differentially expressed consistently (either always above or always below the thresholds) among the three susceptible isolates and were not differentially expressed among the three resistant isolates were defined as candidate markers.

### Example 5: Copies/Cell Measurements

To measure copies per cell using RNA sequencing data, 2uL of (1/1000 dilution) ERCC RNA Spike-In Mix (Thermo Fisher Scientific, Waltham, MA, USA) was added to the lysis buffer in the RNeasy Mini Kit to each individual sample. The number of copies of each ERCC transcript in the sample was calculated, by accounting for dilution and multiplying by Avogadro’s number (manufacturer’s concentrations were reported in attomoles/µL). The relationship between log₂(ERCC copies added) against log₂(gene expression in TPM) was plotted and a linear regression in the region of linearity was performed. The linear regression was used to convert TPM values to total RNA copies in each sample. Finally, using the CFU measured for each sample from plating (described in the “Antibiotic exposure for RNA sequencing” section), the total RNA copies were converted to copies per cell.

### Example 6: Validation With Droplet Digital PCR (dPCR)

Primers were designed for candidate markers using Primer-BLAST[13] and primer alignments were verified using SnapGene. Expression of candidate markers was quantified using the Bio-Rad QX200 droplet dPCR system (Bio-Rad Laboratories, Hercules, CA, USA). The concentration of the components in the dPCR mix used in this study were as follows: 1× EvaGreen Droplet Generation Mix (Bio-Rad), 150U/mL WarmStart RTx Reverse Transcriptase, 800U/mL RiboGaurd RNase Inhibitor, 500 nM forward primer, and 500 nM reverse primer. The RNA extraction comprised 5% of the final volume in the dPCR mix.

For each isolate, candidate marker expression was quantified in the control and treated samples and the fold-change difference (C:T ratio) was calculated. To account for potential differences between the control and treated samples that could arise from experimental variability and extraction efficiency, ribosomal RNA (rRNA) was used as an internal control.

From the sequencing data, it was found that rRNA was not affected by antibiotic exposure in the time frame of this study and showed very low variability. The 16S rRNA in the control was therefore also quantified, samples were treated by dPCR and an rRNA C:T ratio was calculated. The C:T ratio of each marker was normalized with the rRNA C:T ratio. All dPCR C:T ratios reported in the example section of the disclosure are the normalized C:T ratios.

### Example 7: Temporal Shifts in Global Gene Expression Upon Antibiotic Exposure

RNA-seq was used to study the transcriptome response of susceptible and resistant isolates of N. gonorrhoeae after 5, 10, and 15 min of ciprofloxacin exposure (FIG. 1). Each clinical isolate was initially split into two tubes, where one tube was exposed to the antibiotic (+) and the other served as the control with no antibiotic exposure (-). Samples were collected for RNA-seq prior to antibiotic exposure (time zero) and every 5 min for 15 min. The fold change in gene expression was calculated between the control and treated samples, which is defined as the control:treated ratio (C:T ratio).

Genes that demonstrated significant fold-change differences between the susceptible and resistant isolates were identified as differentially expressed. To account for biological variability, three pairs of susceptible and resistant isolates were used in this study to identify markers. Candidate markers were selected from the pool of differentially expressed genes and were validated using droplet dPCR (see Examples 4 and 6).

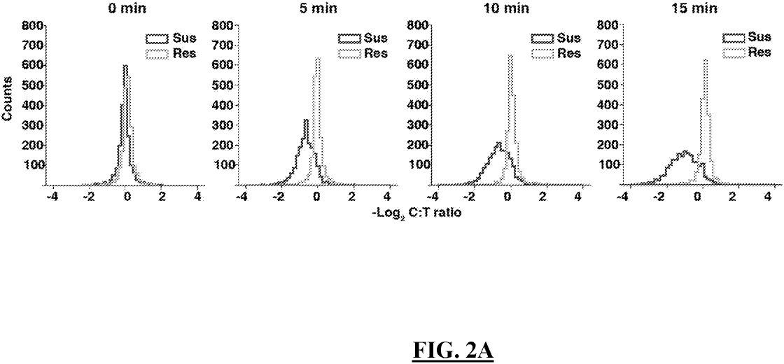

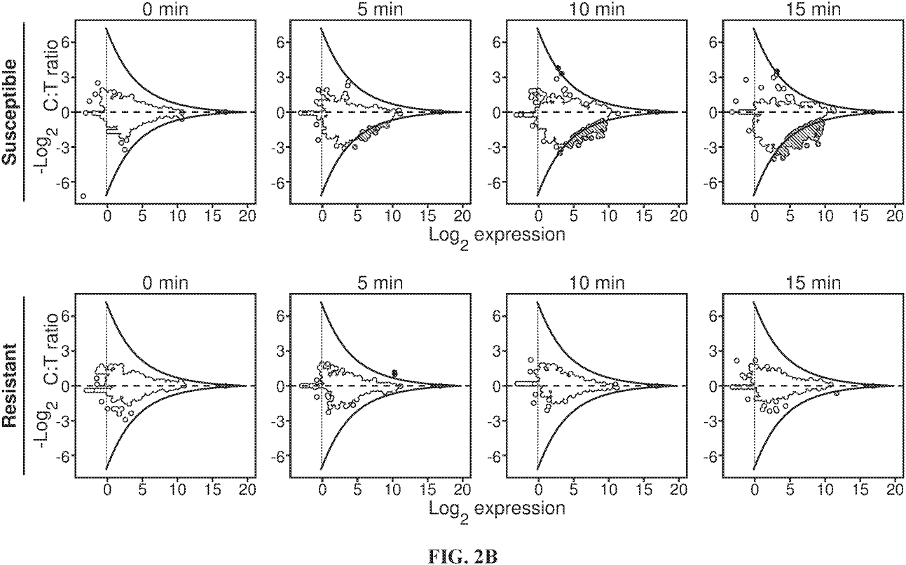

Global shifts were observed in RNA expression in susceptible isolates in as early as 5 min after antibiotic exposure (FIG. 2A). The distribution of fold changes in gene expression levels (C:T ratios) indicated global shifts toward negative log₂ fold-change values (downregulation). The magnitude of fold change at which most genes were distributed was approximately 2-fold. The tail of the distribution illustrates that a few genes responded to antibiotic exposure with changes as large as 6-fold within 5 min. Increasing the antibiotic exposure time further shifted the distribution to larger negative log₂ fold-change values. The transcriptional response in resistant isolates was tightly distributed around negative log₂ zero values at all time points, indicating that the transcriptome did not significantly respond to antibiotic in the resistant isolates (FIG. 2A).

To identify genes that were differentially expressed between control and treated samples, a threshold of significance was defined (FIG. 2B). The threshold of significance took into account technical variability and was calculated from the C:T ratios at t = 0 min of all biological replicates that had RNA sequenced (three susceptible and three resistant isolates). For each of the six gene expression datasets (one for each isolate), the -log₂(C:T ratio) was plotted against the -log₂(expression) for all genes and a negative exponential curve was fit to the outer edge of each plot.

The curves were then averaged from all six datasets and added a 90% confidence interval to the average curve by assuming a Gaussian fit for the error distribution, which was defined as the threshold of significance. Genes with a -log₂(C:T ratio) value above or below the upper and lower thresholds were identified as differentially expressed. Downregulated genes (fold changes below the significance threshold) appeared as early as 5 min after antibiotic exposure (blue dots, FIG. 2B). Two upregulated genes (above the significance threshold) appeared after 10 min of exposure (orange dots, FIG. 2B).

A key aim of this study was to identify RNA markers that would yield a measurable response after only a short antibiotic exposure (less or equal to 15 min) to ensure this approach can fit within the required timescale for a rapid AST. It is possible that longer exposure times could provide additional insight into the biological response of N. gonorrhoeae to ciprofloxacin, but this was not the focus of this study. Furthermore, the short exposure times potentially introduce a bias toward transcripts present at low abundance when evaluating fold change.

For transcripts present at high abundance to display the same fold change, a substantially higher number of mRNA molecules must be transcribed, which would require longer timescales. As an example, a 4-fold change from 1 to 4 transcripts requires 3 additional mRNA to be produced, whereas a 4-fold change from 20 to 80 requires 60 mRNA to be transcribed. This bias also holds true in downregulation, where mRNA continues to be transcribed in the control samples, whereas transcript levels drop in treated samples due to degradation of RNA, and/or a reduction in rate of transcription.

### Example 8: Selection of Candidate Markers That Are Consistent in Response and Abundant

RNA expression in response to antibiotics can be heterogeneous among different isolates of the same species[26]; thus, it is important to select candidate markers from differentially expressed genes that respond consistently across isolates of N. gonorrhoeae.

To identify these markers, three different pairs of susceptible isolates (minimum inhibitory concentrations (MICs) <= 0.015microg/mL) and resistant isolates (MICs 2.0 microgram/mL, 4.0 microgram/mL, and 16.0 microgram/mL) were exposed to ciprofloxacin for 15 min and extracted RNA for sequencing (see workflow in FIG. 1).

The nature of the transcriptional response of N. gonorrhoeae to antibiotic exposure was a global downregulation in transcript levels. In particular, 181, 41, and 410 differentially expressed genes were found in susceptible isolates 1, 2, and 3, respectively (FIG. 3A).

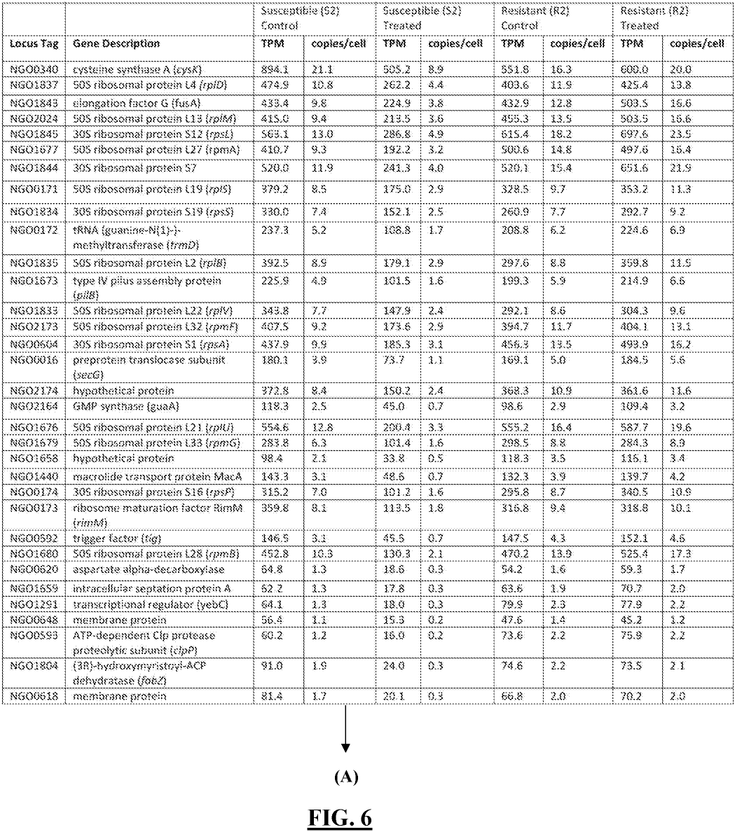

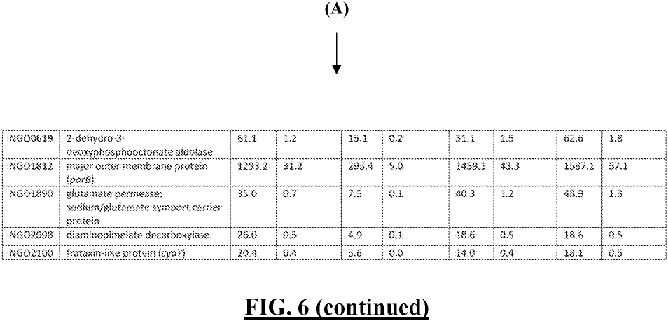

Among the differentially expressed genes, 38 genes responded consistently across the three pairs of susceptible and resistant isolates (i.e. responses overlapped in all three susceptible isolates and were not responsive in all three resistant isolates) (see FIG. 6).

Among the 38 candidate markers, 15 were ribosomal proteins (including one of the top markers, rpmB), which play a prominent role in assembly and function of the ribosomes and are essential for cell growth. Mutations in ribosomal proteins have been reported to confer resistance to different classes of antibiotics[27].

These 38 genes spanned a variety of biochemical functions in the cell. Six candidate transcript markers were selected for further analysis based on the following criteria: (1) high fold change; (2) high expression levels (>75 transcripts per million, TPM); and (3) representative of different biochemical pathways.

The selected candidate markers were: porB (membrane protein), rpmB (ribosomal protein), tig (molecular chaperone), yebC (transcriptional regulator), pilB (pilus assembly ATPase), and cysK (cysteine synthase). Among the candidate marks, all exhibited downregulation in response to ciprofloxacin.

The candidate marker with the highest abundance and largest fold change upon antibiotic exposure was porB, which is a membrane channel forming protein and the site of antibiotic influx into the cell[28]. porB is a porin protein responsible for uptake of small nutrients and the site of antibiotic influx into the cell. The expression of porins is highly regulated in response to environmental stimuli[29]. Reducing permeability to decrease intracellular antibiotic concentration is a known mechanism for bacteria to confer antibiotic resistance[27]. The downregulation of porB observed in this study can be attributed to a halt in growth processes caused by ciprofloxacin damage and possibly an attempt to reduce influx of antibiotic.

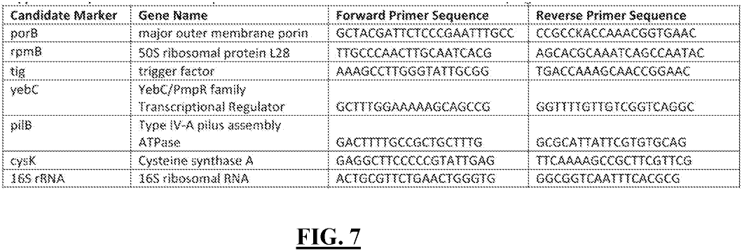

A high level of gene expression was one of the criteria for selection of candidate markers from the sequencing data. High expression of candidate markers is not only important for sensitivity and limits of detection, as has been previously demonstrated in AST methods based on quantification of DNA replication[30], but is particularly important for clinical samples with low numbers of pathogen cells. One of the advantages of RNA compared with DNA as a nucleic acid marker is its natural abundance in the cell. Because the gene expression values obtained from sequencing are relative values, the next step was to quantify the absolute copies per cell for the candidate markers. In the quantification approach, clinical isolate samples were plated after 15 min of ciprofloxacin exposure to obtain cell numbers in colony forming units (CFU/mL). Primers were designed for the candidate markers (see Example 6 and FIG. 7) and measured their absolute concentration using dPCR. The concentrations were converted to per cell values using the cell counts from plating (FIG. 3B).

Additionally, the RNA sequencing data was used to obtain transcriptome-wide estimates of transcript copies per cell. In the sequencing approach, external RNA control consortium (ERCC) spike-ins was added to the lysis buffer step of the extraction protocol in order to capture any loss of RNA throughout the extraction steps. By linear regression the relationship between ERCC copies added to the samples and ERCC quantified by sequencing was captured. Using the linear regression, gene expression values were converted from RNA sequencing (in TPM) to approximate copy numbers per cell (see Example 5). The transcript copies per cell estimated for the candidate markers using the sequencing approach were within the same order of magnitude as the absolute copies per cell measured by digital PCR (FIG. 3B).

It is noted that gyrA and parC, which are known genotypic markers for resistance to ciprofloxacin, were not found to be differentially expressed. recA, which is one of the prominent genes in the SOS response, was also not found to have an increased transcript level because N. gonorrhoeae does not have a true SOS system[31, 32]. Whereas recA is a specific cellular response to overcome DNA damage, the global downregulation that was observed suggests a general shift away from growth and cell proliferation

### Example 9: Validation of Markers by dPCR

To determine how the relative changes observed through RNA-seq compare to direct gene expression measurements by dPCR, dPCR assays were designed for candidate markers, which involved measuring the expression of the candidate marker in both control and treated samples, and calculating the C:T ratio.

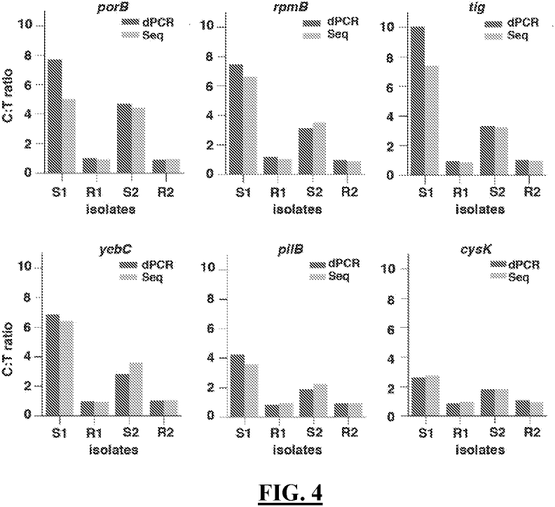

In this assay, the 16S rRNA was also measured and used to normalize the C:T ratio of the candidate markers. In the three susceptible isolates that were sequenced we found that rRNA consistently showed the smallest fold change (< 1.06) in response to ciprofloxacin compared with all other genes in N. gonorrhoeae. Therefore, to account for experimental variations in the antibiotic exposure and RNA extraction steps between control and treated samples, the 16S rRNA was used as an intracellular control for normalizing the C:T ratios (see Example 6). It was found that the C:T ratios measured by the dPCR assay agreed with the C:T ratios obtained through sequencing (FIG. 4), confirming that both approaches accurately capture the transcriptional response to antibiotic exposure.

### Example 10: Validation of RNA Markers porB and rpmB Across CDC Isolates

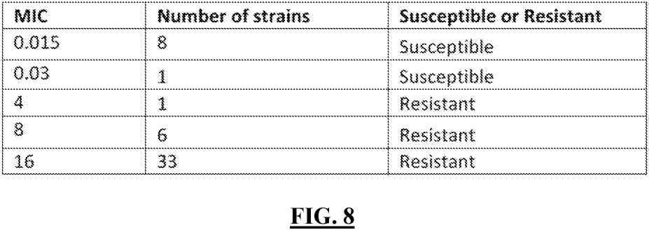

To determine whether candidate markers respond consistently across a large pool of isolates with genetic variability, the two candidate markers with the highest abundance and fold change (porB and rpmB) were chosen to determine the susceptibility of 49 clinical isolates, with a wide range of MIC values (see FIG. 8), from the N. gonorrhoeae panel of the Centers for Disease Control (CDC) Antimicrobial Resistance Isolate Bank.

The MIC values were representative of the population-wide distribution values reported by the European Committee on Antimicrobial Susceptibility Testing[34]. Each clinical isolate was exposed to ciprofloxacin for 10 min and the fold change was measured in expression of the two candidate markers between the control and treated sample using dPCR (FIG. 5). The results show that both markers correctly classified all 49 CDC isolates, based on Clinical and Laboratory Standards Institute (CLSI) breakpoint values, as 9 susceptible and 40 resistant strains.

In particular, both markers were consistent in their ability to correctly determine susceptibility or resistance of all 49 clinical isolates. porB demonstrated C:T ratios between 2.5 to 7 and rpmB demonstrated C:T ratios between 2 and 6 after 10 min of antibiotic exposure in the nine susceptible clinical isolates. The large fold changes highlight the significance of using RNA response as an AST marker compared with quantification of DNA replication. The previous work using dPCR quantification of DNA replication demonstrated C:T ratios between 1.2 and 2.4 for 15 min of antibiotic exposure in susceptible E. coli[30], which has a doubling time approximately 3 times shorter than N. gonorrhoeae.

An alignment search of porB was performed against other prokaryotes and porB was found to be specific to the Neisseria genus. AST markers should be specific to the pathogen of interest because additional bacterial species are likely to be present in clinical samples.

### Example 11: DNA Quantification of N. Gonorrhoeae

Antibiotic susceptible and resistant clinical isolates plated from glycerol stocks onto Chocolate Agar plates and grown in static incubation overnight (37° C., 5% CO2). Cells were re-suspended in Hardy Fastidious Broth (HFB) and incubated for 45 min (37° C., 5% CO2) with shaking (800 rpm) to an OD600 between 1 and 5. Cultures were diluted (5X) into HFB. Each isolate culture was split into “treated” and “control” tubes. Ciprofloxacin was added to the “treated” tubes (final concentration of 0.5 µg/mL) and water was added to the “control” tubes; cultures were incubated (static; 37° C., 5% CO2) for 15 min. Samples for DNA quantification were extracted at 0 and 15 min using the Epicentre QuickExtract DNA Extraction Solution according to the manufacturer’s protocol. 10 uL of sample is placed into 90 uL extraction buffer and incubated at 65° C. for 6 min, followed by 98° C. for 4 min. t0 samples were left at 65° C. during treatment. DNA quantification was performed by digital droplet PCR. The concentrations of the components in the dPCR mix was as follows: 1× QX200 ddPCR EvaGreen Supermix (Bio-Rad), 500 nM forward primer GTTTCAGCGGCAGCATTCA (SEQ ID NO: 176), and 500 nM reverse primer CCGGAACTGGTTTCATCTGATT (SEQ ID NO: 177). Primers that target the 16S or 23S gene of N. gonorrhoeae can be used for dPCR amplification.

### Example 12: porB Sequences in 50 Clinical Isolates From the CDC Bank

In order to understand the variability of the porB gene among the 50 CDC clinical isolates, a clustal omega alignment was performed to determine the smallest percent identity between the FA 1090 sequence and the 50 CDC sequences. The percent identity was shown to be 94.94%. porB is known to be more variable than rpmB and therefore it is likely that percent identity will be higher for rpmB. The porB sequences for the 50 clinical isolates from the CDC bank are listed in ANNEX D (SEQ ID NO: 178-227).

### Example 13: Determination of Antibiotic MIC in Targeted Microorganism

An antibiotic MIC in a targeted organism can be determined in connection with any one of the methods herein described.

For example, when determining ciprofloxacin MIC in Neisseria gonorrhoeae, in some embodiments samples would be treated at 0.015, 0.030, 0.060, 0.125, 0.25, 0.5, 1.0, 2.0, and 4.0 microgram/mL. The C:T ratios measured at each concentration would then be used to determine the sample’s MIC. MIC could be determined, for example, by fitting a curve to the C:T ratios obtained at each concentration of antibiotic plotted vs the concentration of antibiotic used for treatment, and determining the concentration at which the maximum slope of the curve occurs.

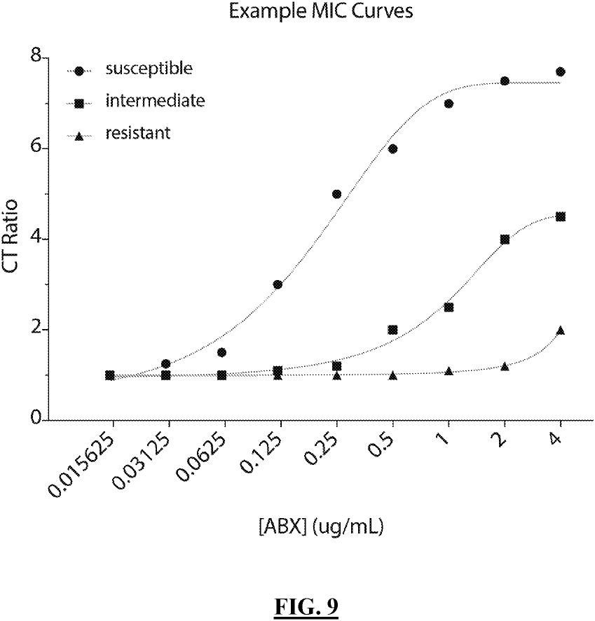

This concentration of antibiotic would then correlate to a particular MIC, determined from performing this method on samples with known MICs. MIC could also be determined by the value at which the fit curve crosses a pre-defined threshold or from the lowest antibiotic concentration that gives a CT response larger than a pre-defined threshold. MIC could also be determined from matching the shape of single curve (or multiple curves) fit to the CT ratios to a pre-constructed library of curves determined by performing the method on isolates with known MICs. An exemplary curve fitting antibiotic concentrations and C:T ratios is reported in FIG. 9.

### Example 14: Determination of Type of Degree of Antibiotic Susceptibility in Targeted Microorganism

In order to determine if a sample contains bacteria with intermediate susceptibility, susceptible bacteria, or resistant bacteria to the antibiotic of interest, the sample can be exposed to three concentrations of antibiotic: a concentration equal to the susceptible MIC breakpoint, a concentration equal to the concentration of the resistant MIC breakpoint, and a concentration equal to the average of the maximum and minimum of the intermediate MIC breakpoint range. Susceptibility would then be determined , for example, by measuring the slope obtained by fitting a curve or line to the three points on the C:T ratio vs treatment concentration plot, and/or by comparing the relative difference in C:T ratio between the low and intermediate concentration of antibiotic and the difference in CT ratio between the intermediate and high concentration, and/or by comparing the magnitude of the value relative to a pre-defined threshold, or a combination of these analyses. For example, for exposure or treatment of Neisseria gonorrhoeae to ciprofloxacin the sample would be exposed to 0.06, 0.25, and 1.0 ug/mL ciprofloxacin.

### Example 15: Detection of Antibiotic Susceptibility of a N Gonorrhoeae Using an N. Gonorrhoeae RNA Marker of the Disclosure (Prophetic)

This example follows the procedure used in [30] Schoepp, N.G., et al., Rapid pathogen-specific phenotypic antibiotic susceptibility testing using digital LAMP quantification in clinical samples. Sci Transl Med, 2017. 9(410)). Urine containing or suspected of containing Neisseria gonorrhoeae is obtained from a patient. Urine is then mixed and incubated in exposure media with and without antibiotics. After incubation in exposure media, nucleic acids are extracted and the target Neisseria gonorrhoeae RNA marker is quantified using digital loop-mediated isothermal amplification (dLAMP). The marker concentration in the control sample (sample without antibiotics) is divided by the concentration in the treated sample (sample with antibiotics) to generate a control-treated ratio (C:T ratio).

If the C:T ratio is above the threshold, Neisseria gonorrhoeae bacteria from this patient sample are called susceptible. If the C:T ratio is below the threshold, Neisseria gonorrhoeae bacteria from this patient sample are called resistant. If the C:T ratio is at the threshold, or within 0.05 of the threshold, Neisseria gonorrhoeae bacteria from this patient sample are called indeterminate.

### Example 16: An Exemplary Performance Standard for Antimicrobial Susceptibility Testing According to CISI Standard

The following description is taken from Clinical Laboratory Standards Institute (CISI) as an example for performing an Antibiotic Susceptibility Test (AST) as well as breakpoint MIC values for various bacteria according to the CLSI standard. More detailed description and updates for CLSI documents can be further found at https://clsi.org/standards-development/documentcorrection-notices/ as will be understood by a person skilled in the art.

TABLE 8

below shows an exemplary zone diameter and MIC breakpoints for Neisseria gonorrhoease.
Table 8: An exemplary zone diameter and MIC breakpoints for Neiseeria gonorrhoeae

| Testing Conditions | | Routine QC Recommendations (see Tables 4B and 5C of the CLSI document at https://Clsi.org/standards/products/mi crobiology/documents/m100/for acceptable QC ranges) N. gonorrhoeae ATCCⓇ* 49226 When a commercial test system is used for susceptibility testing, refer to the manufacturer’s instructions for |
|---|---|---|
| Medium | Disk diffusion: GC agar base and 1% defined growth supplement. (The use of a cysteine-free growth supplement is not required for disk diffusion testing.) Agar dilution: GC agar base and 1% defined growth supplement. (The use of a cysteine-free growth supplement is required for agar dilution tests with carbapenems and clavulanate. Cysteine-containing defined growth supplement | |
| | does not significantly alter dilution test results with other drugs.) | QC test recommendations and QC ranges. |
| Inoculum | Colony suspension, equivalent to a 0.5 McFarland standard prepared in MHB or 0.9% phosphate-buffered saline, pH 7, using colonies from an overnight (20- to 24-hour) chocolate agar plate incubated in 5% CO2 | |
| Incubation | 36° C. ± 1° C. (do not exceed 37° C.); 5% CO2; all methods, 20-24 hours | |

* ATCC® is a registered trademark of the American Type Culture Collection

General Comments include:
(1) For disk diffusion, test a maximum of 9 disks on a 150-mm plate and 4 disks on a 100-mm plate. For some agents, eg, fluoroquinolones or cephalosporins, only 2 to 3 disks may be tested per plate. Measure the diameter of the zones of complete inhibition (as judged by the unaided eye), including the diameter of the disk. Hold the Petri plate a few inches above a black background illuminated with reflected light. The zone margin should be considered the area showing no obvious, visible growth that can be detected with the unaided eye. Ignore faint growth of tiny colonies that can be detected only with a magnifying lens at the edge of the zone of inhibited growth.
(2) The clinical effectiveness of cefmetazole, cefotetan, cefoxitin, and spectinomycin for treating infections due to organisms that produce intermediate results with these agents is unknown.
(3) For disk diffusion testing of N. gonorrhoeae, an intermediate result for an antimicrobial agent indicates either a technical problem that should be resolved by repeat testing or a lack of clinical experience in treating infections due to organisms with these zones. Strains with intermediate zones to agents other than cefmetazole, cefotetan, cefoxitin, and spectinomycin have a documented lower clinical cure rate (85% to 95%) compared with > 95% for susceptible strains.
(4) The recommended medium for testing N. gonorrhoeae consists of GC agar to which a 1% defined growth supplement (1.1 g L-cystine, 0.03 g guanine HCl, 0.003 g thiamine HCl, 0.013 g para-aminobenzoic acid, 0.01 g B12, 0.1 g cocarboxylase, 0.25 g NAD, 1 g adenine, 10 g L-glutamine, 100 g glucose, 0.02 g ferric nitrate, 25.9 g L-cysteine HCl [in 1L H2O]) is added after autoclaving.

TABLE 9

Table 9: A list of exemplary antibiotics and their zone diameter and MIC breakpoints

| Test/Repo rt Group | Antimicrobia 1 Agent | Disk Cont ent | Interpretive Categories and Zone Diameter Breakpoints, nearest whole mm | | | Interpretive Categories and MIC Breakpoints, µg/mL | | | Comments |
|---|---|---|---|---|---|---|---|---|---|
| | | | S | I | R | S | I | R | |
| PENICILLINS | | | | | | | | | |
| O | Penicillin | 10 units | ≥ 47 | 27-46 | ≤ 26 | ≤ 0.06 | 0.12-1 | ≥2 | See general comment (3). (5) A positive β-lactamase test predicts resistance to penicillin, ampicillin, and amoxicillin. (6) A β-lactamase test detects one form of penicillin resistance in N. gonorrhoeae and also may be used to provide epidemiological information. Strains with chromosomally mediated resistance can be detected only by the disk diffusion method or the agar dilution MIC method. (7) Gonococci that produce zones of inhibition of ≤ 19 mm around a 10-unit penicillin disk are likely to be β-lactamase-producing strains. However, the β-lactamase test remains preferable to other susceptibility methods for rapid, accurate recognition of this plasmid-mediated penicillin resistance. |
| CEPHEMS (PARENTERAL) (Including cephalosporins I, II, III, and IV. Please refer to Glossary I.) | | | | | | | | | |
| A | Ceftriaxone | 30 µg | ≥ 35 | - | - | ≤ 0.25 | - | - | |
| O | Cefoxitin | 30 µg | ≥ 28 | 24-27 | ≤ 23 | ≤2 | 4 | ≥8 | See general comment (2) |
| O | Cefuroxime | 30 µg | ≥31 | 26-30 | ≤ 25 | ≤1 | 2 | ≥4 | See general comment (2) |
| O | Cefepime | 30 µg | ≥31 | - | - | ≤ 0.5 | - | - | |
| O | Cefmetazole | 30 µg | ≥ 33 | 28-32 | ≤ 27 | ≤2 | 4 | ≥8 | See general comment (2) |
| O | Cefotaxime | 30 µg | ≥31 | - | - | ≤ 0.5 | - | - | |
| O | Cefotetan | 30 µg | ≥26 | 20-25 | ≤19 | ≤2 | 4 | ≥8 | See general comment (2) |
| O | Ceftazidime | 30 µg | ≥31 | - | - | ≤ 0.5 | - | - | |
| O | Ceftizoxime | 30 µg | ≥38 | - | - | ≤ 0.5 | - | - | |
| A | Cefixime | 5 µg | ≥31 | - | - | ≤ 0.25 | - | - | |
| O | Cefpodoxime | 10 µg | ≥29 | | - | ≤ 0.5 | - | - | |
| Inv. | Cefetamet | 10 µg | ≥31 | - | - | ≤ 0.5 | - | - | |
| A | Tetracycline | 30 µg | ≥38 | 31-37 | ≤ 30 | ≤ 0.25 | 0.5-1 | ≥2 | (9) Gonococci with 30-µg tetracycline disk zone diameters of ≤ 19 mm usually indicate a plasmid-mediated tetracycline-resistant N. gonorrhoeae isolate. Resistance in these strains should be confirmed by a dilution test (MIC ≥16 µg/mL). |
| A | Ciprofloxacin | 5 µg | ≥41 | 28-40 | ≤ 27 | ≤ 0.06 | 0.12-0.5 | ≥1 | |
| O | Enoxacin | 10 µg | ≥36 | 32-35 | ≤ 31 | ≤ 0.5 | 1 | ≥2 | |
| O | Lomefloxacin | 10 µg | ≥38 | 27-37 | ≤ 26 | ≤ 0.12 | 0.25-1 | ≥2 | |
| O | Ofloxacin | 5 µg | ≥31 | 25-30 | ≤ 24 | ≤ 0.25 | 0.5-1 | ≥2 | |
| Inv. | Fleroxacin | 5 µg | ≥35 | 29-34 | ≤ 28 | ≤ 0.25 | 0.5 | ≥1 | |
| O | Spectinomycin | 100 µg | ≥18 | 15-17 | ≤14 | ≤ 32 | 64 | ≥ 128 | See general comment (2) |

Abbreviations: ATCC®, American Type Culture Collection; I, intermediate; MHB, Mueller-Hinton broth; MIC, minimal inhibitory concentration; QC, quality control; NAD, nicotinamide adenine dinucleotide; R, resistant; S, susceptible.

The examples set forth above are provided to give those of ordinary skill in the art a complete disclosure and description of how to make and use the embodiments of the materials, compositions, systems and methods of the disclosure, and are not intended to limit the scope of what the inventors regard as their disclosure. Those skilled in the art will recognize how to adapt the features of the exemplified methods and systems based on the RNA markers identified herein for detection of susceptibility and resistance against various antibiotics in antimicrobial-resistance bacteria according to various embodiments and scope of the claims.

All patents and publications mentioned in the specification are indicative of the levels of skill of those skilled in the art to which the disclosure pertains.

The entire disclosure of each document cited (including webpages patents, patent applications, journal articles, abstracts, laboratory manuals, books, or other disclosures) in the Background, Summary, Detailed Description, and Examples is hereby incorporated herein by reference. All references cited in this disclosure, including references cited in any one of the Appendices, are incorporated by reference to the same extent as if each reference had been incorporated by reference in its entirety individually. However, if any inconsistency arises between a cited reference and the present disclosure, the present disclosure takes precedence. Furthermore, the computer readable form of the sequence listing of the ASCII text file named “P2255-US-2021-08-23-Sq-List-ST25”, created on Aug. 23, 2021, and having a file size (not “size on disk”) of 425 kilobytes measured on Windows Server 2016 Standard ver. 1607, is incorporated herein by reference in its entirety.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified may be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein may be excluded from a claim of this disclosure. The disclosure illustratively described herein suitably may be practiced in the absence of any element or elements, limitation or limitations which is not specifically disclosed herein.

A number of embodiments of the disclosure have been described. The specific embodiments provided herein are examples of useful embodiments of the invention and it will be apparent to one skilled in the art that the disclosure can be carried out using a large number of variations of the devices, device components, methods steps set forth in the present description. As will be obvious to one of skill in the art, methods and devices useful for the present methods may include a large number of optional composition and processing elements and steps.

In particular, it will be understood that various modifications may be made without departing from the spirit and scope of the present disclosure. Accordingly, other embodiments are within the scope of the following claims.

### REFERENCES

1. Song, J. and C. Yi, Chemical Modifications to RNA: A New Layer of Gene Expression Regulation. ACS Chem Biol, 2017. 12(2): p. 316-325.
2. Jackman, J.E. and J.D. Alfonzo, Transfer RNA modifications: Nature’s combinatorial chemistry playground. Wiley interdisciplinary reviews. RNA, 2013. 4(1): p. 35-48.
3. Wagner, G.P., K. Kin, and V.J. Lynch, Measurement of mRNA abundance using RNA-seq data: RPKM measure is inconsistent among samples. Theory Biosci, 2012. 131(4): p. 281-5.
4. Conesa, A., et al., A survey of best practices for RNA-seq data analysis. Genome Biol, 2016. 17: p. 13.
5. Devore, J.L., Probability and Statistics for Engineering and the Sciences. 9th ed. 2016: Cengage.
6. Kreutz, J.E., et al., Theoretical design and analysis of multivolume digital assays with wide dynamic range validated experimentally with microfluidic digital PCR. Anal Chem, 2011. 83(21): p. 8158-68.
7. Badshah, S.L. and A. Ullah, New developments in non-quinolone-based antibiotics for the inhibiton of bacterial gyrase and topoisomerase IV. Eur J Med Chem, 2018. 152: p. 393-400.
8. Collin, F., S. Karkare, and A. Maxwell, Exploiting bacterial DNA gyrase as a drug target: current state and perspectives. Appl Microbiol Biotechnol, 2011. 92(3): p. 479-97.
9. Chakravorty, S., et al., A detailed analysis of 16S ribosomal RNA gene segments for the diagnosis of pathogenic bacteria. J Microbiol Methods, 2007. 69(2): p. 330-9.
10. Altschul, S.F., et al., Gapped BLAST and PSI-BLAST: a new generation of protein database search programs. Nucleic Acids Res, 1997. 25(17): p. 3389-402.
11. Altschul, S.F., et al., Basic local alignment search tool. J Mol Biol, 1990. 215(3): p. 403-10.
12. Tatusova, T.A. and T.L. Madden, BLAST 2 Sequences, a new tool for comparing protein and nucleotide sequences. FEMS Microbiol Lett, 1999. 174(2): p. 247-50.
13. Ye, J., et al., Primer-BLAST: a tool to design target-specific primers for polymerase chain reaction. BMC bioinformatics, 2012. 13(1): p. 134.
14. Smith, T.F. and M.S. Waterman, Identification of common molecular subsequences. J Mol Biol, 1981. 147(1): p. 195-7.
15. Pearson, W.R., Searching protein sequence libraries: comparison of the sensitivity and selectivity of the Smith-Waterman and FASTA algorithms. Genomics, 1991. 11(3): p. 635-50.
16. Pearson, W.R. and D.J. Lipman, Improved tools for biological sequence comparison. Proc Natl Acad Sci U S A, 1988. 85(8): p. 2444-8.
17. Johnson, L.S., S.R. Eddy, and E. Portugaly, Hidden Markov model speed heuristic and iterative HMM search procedure. BMC Bioinformatics, 2010. 11: p. 431.
18. Gootenberg, J.S., et al., Nucleic acid detection with CRISPR-Cas13a/C2c2. Science, 2017. 356(6336): p. 438-442.
19. Myhrvold, C., et al., Field-deployable viral diagnostics using CRISPR-Cas13. Science, 2018. 360(6387): p. 444-448.
20. Gootenberg, J.S., et al., Multiplexed and portable nucleic acid detection platform with Cas13, Cas12a, and Csm6. Science, 2018. 360(6387): p. 439-444.
21. Zheng, G., et al., Efficient and quantitative high-throughput tRNA sequencing. Nat Methods, 2015. 12(9): p. 835-837.
22. Honda, S., et al., Four-leaf clover qRT-PCR: A convenient method for selective quantification of mature tRNA. RNA Biol, 2015. 12(5): p. 501-8.
23. Davidsen, T. and T. Tonjum, Meningococcal genome dynamics. Nat Rev Microbiol, 2006. 4(1): p. 11-22.
24. Davidsen, T., et al., Genetic interactions of DNA repair pathways in the pathogen Neisseria meningitidis. J Bacteriol, 2007. 189(15): p. 5728-37.
25. Marin, M.A., et al., The invasive Neisseria meningitidis MenC CC103 from Brazil is characterized by an accessory gene repertoire. Sci Rep, 2017. 7(1): p. 1617.
26. Gao, Q., et al., Gene expression diversity among Mycobacterium tuberculosis clinical isolates. Microbiology, 2005. 151(1): p. 5-14.
27. Gomez, J.E., et al., Ribosomal mutations promote the evolution of antibiotic resistance in a multidrug environment. Elife, 2017. 6.
28. Quillin, S.J. and H.S. Seifert, Neisseria gonorrhoeae host adaptation and pathogenesis. Nature Reviews Microbiology, 2018.
29. Fernández, L. and R.E. Hancock, Adaptive and mutational resistance: role of porins and efflux pumps in drug resistance. Clinical microbiology reviews, 2012. 25(4): p. 661-681.
30. Schoepp, N.G., et al., Rapid pathogen-specific phenotypic antibiotic susceptibility testing using digital LAMP quantification in clinical samples. Science translational medicine, 2017. 9(410): p. eaal3693.
31. Stohl, E.A., et al., Purification and characterization of the RecA protein from Neisseria gonorrhoeae. PloS one, 2011. 6(2): p. e17101.
32. Schook, P.O., et al., The DNA-binding activity of the Neisseria gonorrhoeae LexA orthologue NG1427 is modulated by oxidation. Molecular microbiology, 2011. 79(4): p. 846-860.
33. Testing, T.E.C.o.A.S., Ciprofloxacin/Neisseria gonorrhoeae International MIC Distribution - Reference Database 2018-04-02.
34. The European Committee on Antimicrobial Susceptibility Testing. Ciprofloxacin/Neisseria gonorrhoeae International MIC Distribution - Reference Database 2018-04-02. 2018; Available from: https://mic.eucast.org/Eucast2/regShow.jsp?Id=35702.

### ANNEX A

Appendix D: List of 16S ribosomal RNA and 23S ribosomal RNA used for normalization

### Sequences for rRNA

> A9Y61_06450: 23S ribosomal RNA (1 of 4 copies) NZ_CP016017.1:1190505-1193403 - Is on the negative strand DNA (- strand): SEQ ID NO: 1

TGAAATGATAGAGTCAAGTGAATAAGTGCATCAGGCGGATGCCTTGGCGATGATAGGCGAC
GAAGGACGTGTAAGCCTGCGAAAAGCGCGGGGGAGCTGGCAATAAAGCAATGATCCCGCG
GTGTCCGAATGGGGAAACCCACTGCATTCTGTGCAGTATCCTAAGTTGAATACATAGGCTTA
GAGAAGCGAACCCGGAGAACTGAACCATCTAAGTACCCGGAGGAAAAGAAATCAACCGAG
ATTCCGCAAGTAGTGGCGAGCGAACGCGGAGGAGCCTGTACGTAATAACTGTCGAGGTAGA
AGAACAAGCTGGGAAGCTTGACCATAGCGGGTGACAGTCCCGTATTCGAAATCTCAACAGC
GGTACTAAGCGTACGAAAAGTAGGGCGGGACACGTGAAATCCTGTCTGAATATGGGGGGAC
CATCCTCCAAGGCTAAATACTCATCATCGACCGATAGTGAACCAGTACCGTGAGGGAAAGG
CGAAAAGAACCCCGGGAGGGGAGTGAAACAGAACCTGAAACCTGATGCATACAAACAGTG
GGAGCGCCCTAGTGGTGTGACTGCGTACCTTTTGTATAATGGGTCAACGACTTACATTCAGT
AGCGAGCTTAACCGGATAGGGGAGGCGTAGGGAAACCGAGTCTTAATAGGGCGATGAGTTG
CTGGGTGTAGACCCGAAACCGAGTGATCTATCCATGGCCAGGTTGAAGGTGCCGTAACAGG
TACTGGAGGACCGAACCCACGCATGTTGCAAAATGCGGGGATGAGCTGTGGGTAGGGGTGA
AAGGCTAAACAAACTCGGAGATAGCTGGTTCTCCCCGAAAACTATTTAGGTAGTGCCTCGAG
CAAGACACTGATGGGGGTAAAGCACTGTTATGGCTAGGGGGTTATTGCAACTTACCAACCCA
TGGCAAACTCAGAATACCATCAAGTGGTTCCTCGGGAGACAGACAGCGGGTGCTAACGTCC
GTTGTCAAGAGGGAAACAACCCAGACCGCCGGCTAAGGTCCCAAATGATAGATTAAGTGGT
AAACGAAGTGGGAAGGCACAGACAGCCAGGATGTTGGCTTAGAAGCAGCCATCATTTAAAG
AAAGCGTAATAGCTCACTGGTCGAGTCGTCCTGCGCGGAAGATGTAACGGGGCTCAAATCT
ATAACCGAAGCTGCGGATGCCGGTTTACCGGCATGGTAGGGGAGCGTTCTGTAGGCTGATG
AAGGTGCATTGTAAAGTGTGCTGGAGGTATCAGAAGTGCGAATGTTGACATGAGTAGCGAT
AAAGCGGGTGAAAAGCCCGCTCGCCGAAAGCCCAAGGTTTCCTACGCAACGTTCATCGGCG
TAGGGTGAGTCGGCCCCTAAGGCGAGGCAGAAATGCGTAGTCGATGGGAAACAGGTTAATA
TTCCTGTACTTGATTCAAATGCGATGTGGGGACGGAGAAGGTTAGGTTGGCAAGCTGTTGGA
ATAGCTTGTTTAAGCCGGTAGGTGGAAGACTTAGGCAAATCCGGGTTTTCTTAACACCGAAG
AAGTGATGACGAGTGTTTACGGACACGAAGCAACCGATACCACGCTTCCAGGAAAAGCCAC
TAAGCTTCAGTTTGAATCGAACCGTACCGCAAACCGACACAGGTGGGCAGGATGAGAATTC
TAAGGCGCTTGAGAGAACTCGGGAGAAGGAACTCGGCAAATTGATACCGTAACTTCGGGAG
AAGGTATGCCCTCTAAGGTTAAGGACTTGCTCCGTAAGCCCCGGAGGGTCGCAGAGAATAG
GTGGCTGCGACTTGTTTATTAAAAACACGAGCACTCTTGCCAACACGAAAGTGGACGTATAG
GGTGTAACGCCTGCCCGGTGCCGGAAGGTTAATTGAAGATGTGCAAGCATCGGATCGAAGC
CCCGGTAAACGGCGGCCGTAACTATAACGGTCCTAAGGTAGCGAAATTCCTTGTCGGGTAAG
TTCCGACCCGCACGAATGGCGTAACGATGGCCACACTGTCTCCTCCCGAGACTCAGCGAAGT
TGAAGTGGTTGTGAAGATGCAATCTACCCGCTGCTAGACGGAAAGACCCCGTGAACCTTTAC
TGTAGCTTTGCATTGGACTTTGAAGTCACTTGTGTAGGATAGGTGGAAGGCTTGGAAGCAAA
GACGCCAGTCTCTGTGGAGTCGTCCTTGAAAATACCACCCTGGTGTCTTTGAGGTTCTAACCC
AGACCCGTCATCCGGGTCGGGGACCGTGCATGGTAGGCAGTTTGACTGGGGCGGTCTCCTCC
CAAAGCGTAACGGAGGAGTTCGAAGGTTACCTAGGTCCGGTCGGAAATCGGACTGATAGTG
CAATGGCAAAAGGTAGCTTAACTGCGAGACCGACAAGTCGGGCAGGTGCGAAAGCAGGAC
ATAGTGATCCGGTGGTTCTGTATGGAAGGGCCATCGCTCAACGGATAAAAGGTACTCCGGG
GATAACAGGCTTGATTCCGCCCAAGAGTTCATATCGACGGCGGAGTTTGGCACCTCGATGTC
GGCTCATCACATCCTGGGGCTGTAGTCGGTCCCAAGGGTATGGCTGTTCGCCATTTTAAAGT
GGTACGTGAGTTGGGTTTAAAACGTCGTGAGACAGTTTGGTCCCTATCTGCAGTGGGCGTTG
GAAGTTTGACGGGGGCTGCTCCTAGTACGAGAGGACCGGAGTGGACGAACCTCTGGTGTAC
CGGTTGTAACGCCAGTTGCATAGCCGGGTAGCTAAGTTCGGAAGAGATAAGCGCTGAAAGC
ATCTAAGCGCGAAACTCGCCTGAAGATGAGACTTCCCTTGCGGTTTAACCGCACTAAAGGGT
CGTTCGAGACCAGGACGTTGATAGGTGGGGTGTGGAAGCGCGGTAACGCGTGAAGCTAACC
CATACTAATTGCCCGTGAGGCTTGACTCT

cDNA: SEQ ID NO: 2

AGAGTCAAGCCTCACGGGCAATTAGTATGGGTTAGCTTCACGCGTTACCGCGCTTCCACACC
CCACCTATCAACGTCCTGGTCTCGAACGACCCTTTAGTGCGGTTAAACCGCAAGGGAAGTCT
CATCTTCAGGCGAGTTTCGCGCTTAGATGCTTTCAGCGCTTATCTCTTCCGAACTTAGCTACC
CGGCTATGCAACTGGCGTTACAACCGGTACACCAGAGGTTCGTCCACTCCGGTCCTCTCGTA
CTAGGAGCAGCCCCCGTCAAACTTCCAACGCCCACTGCAGATAGGGACCAAACTGTCTCACG
ACGTTTTAAACCCAACTCACGTACCACTTTAAAATGGCGAACAGCCATACCCTTGGGACCGA
CTACAGCCCCAGGATGTGATGAGCCGACATCGAGGTGCCAAACTCCGCCGTCGATATGAACT
CTTGGGCGGAATCAAGCCTGTTATCCCCGGAGTACCTTTTATCCGTTGAGCGATGGCCCTTCC
ATACAGAACCACCGGATCACTATGTCCTGCTTTCGCACCTGCCCGACTTGTCGGTCTCGCAGT
TAAGCTACCTTTTGCCATTGCACTATCAGTCCGATTTCCGACCGGACCTAGGTAACCTTCGAA
CTCCTCCGTTACGCTTTGGGAGGAGACCGCCCCAGTCAAACTGCCTACCATGCACGGTCCCC
GACCCGGATGACGGGTCTGGGTTAGAACCTCAAAGACACCAGGGTGGTATTTTCAAGGACG
ACTCCACAGAGACTGGCGTCTTTGCTTCCAAGCCTTCCACCTATCCTACACAAGTGACTTCAA
AGTCCAATGCAAAGCTACAGTAAAGGTTCACGGGGTCTTTCCGTCTAGCAGCGGGTAGATTG
CATCTTCACAACCACTTCAACTTCGCTGAGTCTCGGGAGGAGACAGTGTGGCCATCGTTACG
CCATTCGTGCGGGTCGGAACTTACCCGACAAGGAATTTCGCTACCTTAGGACCGTTATAGTT
ACGGCCGCCGTTTACCGGGGCTTCGATCCGATGCTTGCACATCTTCAATTAACCTTCCGGCAC
CGGGCAGGCGTTACACCCTATACGTCCACTTTCGTGTTGGCAAGAGTGCTCGTGTTTTTAATA
AACAAGTCGCAGCCACCTATTCTCTGCGACCCTCCGGGGCTTACGGAGCAAGTCCTTAACCT
TAGAGGGCATACCTTCTCCCGAAGTTACGGTATCAATTTGCCGAGTTCCTTCTCCCGAGTTCT
CTCAAGCGCCTTAGAATTCTCATCCTGCCCACCTGTGTCGGTTTGCGGTACGGTTCGATTCAA
ACTGAAGCTTAGTGGCTTTTCCTGGAAGCGTGGTATCGGTTGCTTCGTGTCCGTAAACACTCG
TCATCACTTCTTCGGTGTTAAGAAAACCCGGATTTGCCTAAGTCTTCCACCTACCGGCTTAAA
CAAGCTATTCCAACAGCTTGCCAACCTAACCTTCTCCGTCCCCACATCGCATTTGAATCAAGT
ACAGGAATATTAACCTGTTTCCCATCGACTACGCATTTCTGCCTCGCCTTAGGGGCCGACTCA
CCCTACGCCGATGAACGTTGCGTAGGAAACCTTGGGCTTTCGGCGAGCGGGCTTTTCACCCG
CTTTATCGCTACTCATGTCAACATTCGCACTTCTGATACCTCCAGCACACTTTACAATGCACC
TTCATCAGCCTACAGAACGCTCCCCTACCATGCCGGTAAACCGGCATCCGCAGCTTCGGTTA
TAGATTTGAGCCCCGTTACATCTTCCGCGCAGGACGACTCGACCAGTGAGCTATTACGCTTT
CTTTAAATGATGGCTGCTTCTAAGCCAACATCCTGGCTGTCTGTGCCTTCCCACTTCGTTTAC
CACTTAATCTATCATTTGGGACCTTAGCCGGCGGTCTGGGTTGTTTCCCTCTTGACAACGGAC
GTTAGCACCCGCTGTCTGTCTCCCGAGGAACCACTTGATGGTATTCTGAGTTTGCCATGGGTT
GGTAAGTTGCAATAACCCCCTAGCCATAACAGTGCTTTACCCCCATCAGTGTCTTGCTCGAG
GCACTACCTAAATAGTTTTCGGGGAGAACCAGCTATCTCCGAGTTTGTTTAGCCTTTCACCCC
TACCCACAGCTCATCCCCGCATTTTGCAACATGCGTGGGTTCGGTCCTCCAGTACCTGTTACG
GCACCTTCAACCTGGCCATGGATAGATCACTCGGTTTCGGGTCTACACCCAGCAACTCATCG
CCCTATTAAGACTCGGTTTCCCTACGCCTCCCCTATCCGGTTAAGCTCGCTACTGAATGTAAG
TCGTTGACCCATTATACAAAAGGTACGCAGTCACACCACTAGGGCGCTCCCACTGTTTGTAT
GCATCAGGTTTCAGGTTCTGTTTCACTCCCCTCCCGGGGTTCTTTTCGCCTTTCCCTCACGGTA
CTGGTTCACTATCGGTCGATGATGAGTATTTAGCCTTGGAGGATGGTCCCCCCATATTCAGA
CAGGATTTCACGTGTCCCGCCCTACTTTTCGTACGCTTAGTACCGCTGTTGAGATTTCGAATA
CGGGACTGTCACCCGCTATGGTCAAGCTTCCCAGCTTGTTCTTCTACCTCGACAGTTATTACG
TACAGGCTCCTCCGCGTTCGCTCGCCACTACTTGCGGAATCTCGGTTGATTTCTTTTCCTCCG
GGTACTTAGATGGTTCAGTTCTCCGGGTTCGCTTCTCTAAGCCTATGTATTCAACTTAGGATA
CTGCACAGAATGCAGTGGGTTTCCCCATTCGGACACCGCGGGATCATTGCTTTATTGCCAGC
TCCCCCGCGCTTTTCGCAGGCTTACACGTCCTTCGTCGCCTATCATCGCCAAGGCATCCGCCT
GATGCACTTATTCACTTGACTCTATCATTTCA

RNA: SEQ ID NO: 3

UGAAAUGAUAGAGUCAAGUGAAUAAGUGCAUCAGGCGGAUGCCUUGGCGAUGAUAGGCG
ACGAAGGACGUGUAAGCCUGCGAAAAGCGCGGGGGAGCUGGCAAUAAAGCAAUGAUCCC
GCGGUGUCCGAAUGGGGAAACCCACUGCAUUCUGUGCAGUAUCCUAAGUUGAAUACAUA
GGCUUAGAGAAGCGAACCCGGAGAACUGAACCAUCUAAGUACCCGGAGGAAAAGAAAUC
AACCGAGAUUCCGCAAGUAGUGGCGAGCGAACGCGGAGGAGCCUGUACGUAAUAACUGU
CGAGGUAGAAGAACAAGCUGGGAAGCUUGACCAUAGCGGGUGACAGUCCCGUAUUCGAA
AUCUCAACAGCGGUACUAAGCGUACGAAAAGUAGGGCGGGACACGUGAAAUCCUGUCUG
AAUAUGGGGGGACCAUCCUCCAAGGCUAAAUACUCAUCAUCGACCGAUAGUGAACCAGUA
CCGUGAGGGAAAGGCGAAAAGAACCCCGGGAGGGGAGUGAAACAGAACCUGAAACCUGA
UGCAUACAAACAGUGGGAGCGCCCUAGUGGUGUGACUGCGUACCUUUUGUAUAAUGGGU
CAACGACUUACAUUCAGUAGCGAGCUUAACCGGAUAGGGGAGGCGUAGGGAAACCGAGU
CUUAAUAGGGCGAUGAGUUGCUGGGUGUAGACCCGAAACCGAGUGAUCUAUCCAUGGCC
AGGUUGAAGGUGCCGUAACAGGUACUGGAGGACCGAACCCACGCAUGUUGCAAAAUGCG
GGGAUGAGCUGUGGGUAGGGGUGAAAGGCUAAACAAACUCGGAGAUAGCUGGUUCUCCC
CGAAAACUAUUUAGGUAGUGCCUCGAGCAAGACACUGAUGGGGGUAAAGCACUGUUAUG
GCUAGGGGGUUAUUGCAACUUACCAACCCAUGGCAAACUCAGAAUACCAUCAAGUGGUUC
CUCGGGAGACAGACAGCGGGUGCUAACGUCCGUUGUCAAGAGGGAAACAACCCAGACCGC
CGGCUAAGGUCCCAAAUGAUAGAUUAAGUGGUAAACGAAGUGGGAAGGCACAGACAGCC
AGGAUGUUGGCUUAGAAGCAGCCAUCAUUUAAAGAAAGCGUAAUAGCUCACUGGUCGAG
UCGUCCUGCGCGGAAGAUGUAACGGGGCUCAAAUCUAUAACCGAAGCUGCGGAUGCCGGU
UUACCGGCAUGGUAGGGGAGCGUUCUGUAGGCUGAUGAAGGUGCAUUGUAAAGUGUGCU
GGAGGUAUCAGAAGUGCGAAUGUUGACAUGAGUAGCGAUAAAGCGGGUGAAAAGCCCGC
UCGCCGAAAGCCCAAGGUUUCCUACGCAACGUUCAUCGGCGUAGGGUGAGUCGGCCCCUA
AGGCGAGGCAGAAAUGCGUAGUCGAUGGGAAACAGGUUAAUAUUCCUGUACUUGAUUCA
AAUGCGAUGUGGGGACGGAGAAGGUUAGGUUGGCAAGCUGUUGGAAUAGCUUGUUUAAG
CCGGUAGGUGGAAGACUUAGGCAAAUCCGGGUUUUCUUAACACCGAAGAAGUGAUGACG
AGUGUUUACGGACACGAAGCAACCGAUACCACGCUUCCAGGAAAAGCCACUAAGCUUCAG
UUUGAAUCGAACCGUACCGCAAACCGACACAGGUGGGCAGGAUGAGAAUUCUAAGGCGC
UUGAGAGAACUCGGGAGAAGGAACUCGGCAAAUUGAUACCGUAACUUCGGGAGAAGGUA
UGCCCUCUAAGGUUAAGGACUUGCUCCGUAAGCCCCGGAGGGUCGCAGAGAAUAGGUGGC
UGCGACUUGUUUAUUAAAAACACGAGCACUCUUGCCAACACGAAAGUGGACGUAUAGGG
UGUAACGCCUGCCCGGUGCCGGAAGGUUAAUUGAAGAUGUGCAAGCAUCGGAUCGAAGC
CCCGGUAAACGGCGGCCGUAACUAUAACGGUCCUAAGGUAGCGAAAUUCCUUGUCGGGUA
AGUUCCGACCCGCACGAAUGGCGUAACGAUGGCCACACUGUCUCCUCCCGAGACUCAGCG
AAGUUGAAGUGGUUGUGAAGAUGCAAUCUACCCGCUGCUAGACGGAAAGACCCCGUGAA
CCUUUACUGUAGCUUUGCAUUGGACUUUGAAGUCACUUGUGUAGGAUAGGUGGAAGGCU
UGGAAGCAAAGACGCCAGUCUCUGUGGAGUCGUCCUUGAAAAUACCACCCUGGUGUCUUU
GAGGUUCUAACCCAGACCCGUCAUCCGGGUCGGGGACCGUGCAUGGUAGGCAGUUUGACU
GGGGCGGUCUCCUCCCAAAGCGUAACGGAGGAGUUCGAAGGUUACCUAGGUCCGGUCGGA
AAUCGGACUGAUAGUGCAAUGGCAAAAGGUAGCUUAACUGCGAGACCGACAAGUCGGGC
AGGUGCGAAAGCAGGACAUAGUGAUCCGGUGGUUCUGUAUGGAAGGGCCAUCGCUCAAC
GGAUAAAAGGUACUCCGGGGAUAACAGGCUUGAUUCCGCCCAAGAGUUCAUAUCGACGG
CGGAGUUUGGCACCUCGAUGUCGGCUCAUCACAUCCUGGGGCUGUAGUCGGUCCCAAGGG
UAUGGCUGUUCGCCAUUUUAAAGUGGUACGUGAGUUGGGUUUAAAACGUCGUGAGACAG
UUUGGUCCCUAUCUGCAGUGGGCGUUGGAAGUUUGACGGGGGCUGCUCCUAGUACGAGA
GGACCGGAGUGGACGAACCUCUGGUGUACCGGUUGUAACGCCAGUUGCAUAGCCGGGUA
GCUAAGUUCGGAAGAGAUAAGCGCUGAAAGCAUCUAAGCGCGAAACUCGCCUGAAGAUG
AGACUUCCCUUGCGGUUUAACCGCACUAAAGGGUCGUUCGAGACCAGGACGUUGAUAGG
UGGGGUGUGGAAGCGCGGUAACGCGUGAAGCUAACCCAUACUAAUUGCCCGUGAGGCUU
GACUCU

> A9Y61_06465: 16S ribosomal RNA (1 of 4 copies) NZ_CP016017.1:1194001-1195552 - Is on the negative strand DNA (- strand): SEQ ID NO: 4

TGAACATAAGAGTTTGATCCTGGCTCAGATTGAACGCTGGCGGCATGCTTTACACATGCAAG
TCGGACGGCAGCACAGGGAAGCTTGCTTCTCGGGTGGCGAGTGGCGAACGGGTGAGTAACA
TATCGGAACGTACCGGGTAGCGGGGGATAACTGATCGAAAGATCAGCTAATACCGCATACG
TCTTGAGAGGGAAAGCAGGGGACCTTCGGGCCTTGCGCTATCCGAGCGGCCGATATCTGATT
AGCTGGTTGGCGGGGTAAAGGCCCACCAAGGCGACGATCAGTAGCGGGTCTGAGAGGATGA
TCCGCCACACTGGGACTGAGACACGGCCCAGACTCCTACGGGAGGCAGCAGTGGGGAATTT
TGGACAATGGGCGCAAGCCTGATCCAGCCATGCCGCGTGTCTGAAGAAGGCCTTCGGGTTGT
AAAGGACTTTTGTCAGGGAAGAAAAGGCCGTTGCCAATATCGGCGGCCGATGACGGTACCT
GAAGAATAAGCACCGGCTAACTACGTGCCAGCAGCCGCGGTAATACGTAGGGTGCGAGCGT
TAATCGGAATTACTGGGCGTAAAGCGGGCGCAGACGGTTACTTAAGCAGGATGTGAAATCC
CCGGGCTCAACCCGGGAACTGCGTTCTGAACTGGGTGACTCGAGTGTGTCAGAGGGAGGTG
GAATTCCACGTGTAGCAGTGAAATGCGTAGAGATGTGGAGGAATACCGATGGCGAAGGCAG
CCTCCTGGGATAACACTGACGTTCATGTCCGAAAGCGTGGGTAGCAAACAGGATTAGATACC
CTGGTAGTCCACGCCCTAAACGATGTCAATTAGCTGTTGGGCAACTTGATTGCTTGGTAGCG
TAGCTAACGCGTGAAATTGACCGCCTGGGGAGTACGGTCGCAAGATTAAAACTCAAAGGAA
TTGACGGGGACCCGCACAAGCGGTGGATGATGTGGATTAATTCGATGCAACGCGAAGAACC
TTACCTGGTTTTGACATGTGCGGAATCCTCCGGAGACGGAGGAGTGCCTTCGGGAGCCGTAA
CACAGGTGCTGCATGGCTGTCGTCAGCTCGTGTCGTGAGATGTTGGGTTAAGTCCCGCAACG
AGCGCAACCCTTGTCATTAGTTGCCATCATTCGGTTGGGCACTCTAATGAGACTGCCGGTGA
CAAGCCGGAGGAAGGTGGGGATGACGTCAAGTCCTCATGGCCCTTATGACCAGGGCTTCAC
ACGTCATACAATGGTCGGTACAGAGGGTAGCCAAGCCGCGAGGCGGAGCCAATCTCACAAA
ACCGATCGTAGTCCGGATTGCACTCTGCAACTCGAGTGCATGAAGTCGGAATCGCTAGTAAT
CGCAGGTCAGCATACTGCGGTGAATACGTTCCCGGGTCTTGTACACACCGCCCGTCACACCA
TGGGAGTGGGGGATACCAGAAGTAGGTAGGGTAACCGCAAGGAGTCCGCTTACCACGGTAT
GCTTCATGACTGGGGTGAAGTCGTAACAAGGTAGCCGTAGGGGAACCTGCGGCTGGATCAC
CTCCTTTCTA

cDNA: SEQ ID NO: 5

TAGAAAGGAGGTGATCCAGCCGCAGGTTCCCCTACGGCTACCTTGTTACGACTTCACCCCAG
TCATGAAGCATACCGTGGTAAGCGGACTCCTTGCGGTTACCCTACCTACTTCTGGTATCCCCC
ACTCCCATGGTGTGACGGGCGGTGTGTACAAGACCCGGGAACGTATTCACCGCAGTATGCTG
ACCTGCGATTACTAGCGATTCCGACTTCATGCACTCGAGTTGCAGAGTGCAATCCGGACTAC
GATCGGTTTTGTGAGATTGGCTCCGCCTCGCGGCTTGGCTACCCTCTGTACCGACCATTGTAT
GACGTGTGAAGCCCTGGTCATAAGGGCCATGAGGACTTGACGTCATCCCCACCTTCCTCCGG
CTTGTCACCGGCAGTCTCATTAGAGTGCCCAACCGAATGATGGCAACTAATGACAAGGGTTG
CGCTCGTTGCGGGACTTAACCCAACATCTCACGACACGAGCTGACGACAGCCATGCAGCACC
TGTGTTACGGCTCCCGAAGGCACTCCTCCGTCTCCGGAGGATTCCGCACATGTCAAAACCAG
GTAAGGTTCTTCGCGTTGCATCGAATTAATCCACATCATCCACCGCTTGTGCGGGTCCCCGTC
AATTCCTTTGAGTTTTAATCTTGCGACCGTACTCCCCAGGCGGTCAATTTCACGCGTTAGCTA
CGCTACCAAGCAATCAAGTTGCCCAACAGCTAATTGACATCGTTTAGGGCGTGGACTACCAG
GGTATCTAATCCTGTTTGCTACCCACGCTTTCGGACATGAACGTCAGTGTTATCCCAGGAGG
CTGCCTTCGCCATCGGTATTCCTCCACATCTCTACGCATTTCACTGCTACACGTGGAATTCCA
CCTCCCTCTGACACACTCGAGTCACCCAGTTCAGAACGCAGTTCCCGGGTTGAGCCCGGGGA
TTTCACATCCTGCTTAAGTAACCGTCTGCGCCCGCTTTACGCCCAGTAATTCCGATTAACGCT
CGCACCCTACGTATTACCGCGGCTGCTGGCACGTAGTTAGCCGGTGCTTATTCTTCAGGTACC
GTCATCGGCCGCCGATATTGGCAACGGCCTTTTCTTCCCTGACAAAAGTCCTTTACAACCCG
AAGGCCTTCTTCAGACACGCGGCATGGCTGGATCAGGCTTGCGCCCATTGTCCAAAATTCCC
CACTGCTGCCTCCCGTAGGAGTCTGGGCCGTGTCTCAGTCCCAGTGTGGCGGATCATCCTCTC
AGACCCGCTACTGATCGTCGCCTTGGTGGGCCTTTACCCCGCCAACCAGCTAATCAGATATC
GGCCGCTCGGATAGCGCAAGGCCCGAAGGTCCCCTGCTTTCCCTCTCAAGACGTATGCGGTA
TTAGCTGATCTTTCGATCAGTTATCCCCCGCTACCCGGTACGTTCCGATATGTTACTCACCCG
TTCGCCACTCGCCACCCGAGAAGCAAGCTTCCCTGTGCTGCCGTCCGACTTGCATGTGTAAA
GCATGCCGCCAGCGTTCAATCTGAGCCAGGATCAAACTCTTATGTTCA

RNA: SEQ ID NO: 6

UGAACAUAAGAGUUUGAUCCUGGCUCAGAUUGAACGCUGGCGGCAUGCUUUACACAUGC
AAGUCGGACGGCAGCACAGGGAAGCUUGCUUCUCGGGUGGCGAGUGGCGAACGGGUGAG
UAACAUAUCGGAACGUACCGGGUAGCGGGGGAUAACUGAUCGAAAGAUCAGCUAAUACC
GCAUACGUCUUGAGAGGGAAAGCAGGGGACCUUCGGGCCUUGCGCUAUCCGAGCGGCCGA
UAUCUGAUUAGCUGGUUGGCGGGGUAAAGGCCCACCAAGGCGACGAUCAGUAGCGGGUC
UGAGAGGAUGAUCCGCCACACUGGGACUGAGACACGGCCCAGACUCCUACGGGAGGCAGC
AGUGGGGAAUUUUGGACAAUGGGCGCAAGCCUGAUCCAGCCAUGCCGCGUGUCUGAAGA
AGGCCUUCGGGUUGUAAAGGACUUUUGUCAGGGAAGAAAAGGCCGUUGCCAAUAUCGGC
GGCCGAUGACGGUACCUGAAGAAUAAGCACCGGCUAACUACGUGCCAGCAGCCGCGGUAA
UACGUAGGGUGCGAGCGUUAAUCGGAAUUACUGGGCGUAAAGCGGGCGCAGACGGUUAC
UUAAGCAGGAUGUGAAAUCCCCGGGCUCAACCCGGGAACUGCGUUCUGAACUGGGUGACU
CGAGUGUGUCAGAGGGAGGUGGAAUUCCACGUGUAGCAGUGAAAUGCGUAGAGAUGUGG
AGGAAUACCGAUGGCGAAGGCAGCCUCCUGGGAUAACACUGACGUUCAUGUCCGAAAGCG
UGGGUAGCAAACAGGAUUAGAUACCCUGGUAGUCCACGCCCUAAACGAUGUCAAUUAGC
UGUUGGGCAACUUGAUUGCUUGGUAGCGUAGCUAACGCGUGAAAUUGACCGCCUGGGGA
GUACGGUCGCAAGAUUAAAACUCAAAGGAAUUGACGGGGACCCGCACAAGCGGUGGAUG
AUGUGGAUUAAUUCGAUGCAACGCGAAGAACCUUACCUGGUUUUGACAUGUGCGGAAUC
CUCCGGAGACGGAGGAGUGCCUUCGGGAGCCGUAACACAGGUGCUGCAUGGCUGUCGUCA
GCUCGUGUCGUGAGAUGUUGGGUUAAGUCCCGCAACGAGCGCAACCCUUGUCAUUAGUU
GCCAUCAUUCGGUUGGGCACUCUAAUGAGACUGCCGGUGACAAGCCGGAGGAAGGUGGG
GAUGACGUCAAGUCCUCAUGGCCCUUAUGACCAGGGCUUCACACGUCAUACAAUGGUCGG
UACAGAGGGUAGCCAAGCCGCGAGGCGGAGCCAAUCUCACAAAACCGAUCGUAGUCCGGA
UUGCACUCUGCAACUCGAGUGCAUGAAGUCGGAAUCGCUAGUAAUCGCAGGUCAGCAUAC
UGCGGUGAAUACGUUCCCGGGUCUUGUACACACCGCCCGUCACACCAUGGGAGUGGGGGA
UACCAGAAGUAGGUAGGGUAACCGCAAGGAGUCCGCUUACCACGGUAUGCUUCAUGACU
GGGGUGAAGUCGUAACAAGGUAGCCGUAGGGGAACCUGCGGCUGGAUCACCUCCUUUCU
A

> A9Y61_07175: 23S ribosomal RNA (1 of 4 copies) NZ_CP016017.1:1325810-1328708 - Is on the negative strand DNA (- strand): SEQ ID NO: 7

TGAAATGATAGAGTCAAGTGAATAAGTGCATCAGGCGGATGCCTTGGCGATGATAGGCGAC
GAAGGACGTGTAAGCCTGCGAAAAGCGCGGGGGAGCTGGCAATAAAGCAATGATCCCGCG
GTGTCCGAATGGGGAAACCCACTGCATTCTGTGCAGTATCCTAAGTTGAATACATAGGCTTA
GAGAAGCGAACCCGGAGAACTGAACCATCTAAGTACCCGGAGGAAAAGAAATCAACCGAG
ATTCCGCAAGTAGTGGCGAGCGAACGCGGAGGAGCCTGTACGTAATAACTGTCGAGGTAGA
AGAACAAGCTGGGAAGCTTGACCATAGCGGGTGACAGTCCCGTATTCGAAATCTCAACAGC
GGTACTAAGCGTACGAAAAGTAGGGCGGGACACGTGAAATCCTGTCTGAATATGGGGGGAC
CATCCTCCAAGGCTAAATACTCATCATCGACCGATAGTGAACCAGTACCGTGAGGGAAAGG
CGAAAAGAACCCCGGGAGGGGAGTGAAACAGAACCTGAAACCTGATGCATACAAACAGTG
GGAGCGCCCTAGTGGTGTGACTGCGTACCTTTTGTATAATGGGTCAACGACTTACATTCAGT
AGCGAGCTTAACCGGATAGGGGAGGCGTAGGGAAACCGAGTCTTAATAGGGCGATGAGTTG
CTGGGTGTAGACCCGAAACCGAGTGATCTATCCATGGCCAGGTTGAAGGTGCCGTAACAGG
TACTGGAGGACCGAACCCACGCATGTTGCAAAATGCGGGGATGAGCTGTGGGTAGGGGTGA
AAGGCTAAACAAACTCGGAGATAGCTGGTTCTCCCCGAAAACTATTTAGGTAGTGCCTCGAG
CAAGACACTGATGGGGGTAAAGCACTGTTATGGCTAGGGGGTTATTGCAACTTACCAACCCA
TGGCAAACTCAGAATACCATCAAGTGGTTCCTCGGGAGACAGACAGCGGGTGCTAACGTCC
GTTGTCAAGAGGGAAACAACCCAGACCGCCGGCTAAGGTCCCAAATGATAGATTAAGTGGT
AAACGAAGTGGGAAGGCACAGACAGCCAGGATGTTGGCTTAGAAGCAGCCATCATTTAAAG
AAAGCGTAATAGCTCACTGGTCGAGTCGTCCTGCGCGGAAGATGTAACGGGGCTCAAATCT
ATAACCCAAGCTGCGTATGCCGGTTTACCGGCATGGTAGGGGAGCGTTCTGTAGGCTGATGA
AGGTGCATTGTAAAGTGTGCTGGAGGTATCAGAAGTGCGAATGTTGACATGAGTAGCGATA
AAGCGGGTGAAAAGCCCGCTCGCCGCAAAGCCCAAGGTTTCCTACGCAACGTTCATCGGCG
TAGGGTGAGTCGGCCCCTAAGGCGAGGCAGAAATGCGTAGTCGATGGGAAACAGGTTAATA
TTCCTGTACTTGATTCAAATGCGATGTGGGGACGGAGAAGGTTAGGTTGGCAAGCTGTTGGA
ATAGCTTGTTTAAGCCGGTAGGTGGAAGACTTAGGCAAATCCGGGTTTTCTTAACACCGAGA
AGTGATGACGAGTGTCTACGGACACGAAGCAACCGATACCACGCTTCCAGGAAAAGCCACT
AAGCTTCAGTTTGAATCGAACCGTACCGCAAACCGACACAGGTGGGCAGGATGAGAATTCT
AAGGCGCTTGAGAGAACTCGGGAGAAGGAACTCGGCAAATTGATACCGTAACTTCGGGAGA
AGGTATGCCCTCTAAGGTTAAGGACTTGCTCCGTAAGCCCCGGAGGGTCGCAGAGAATAGG
TGGCTGCGACTGTTTATTAAAAACACAGCACTCTGCCAACACGAAAGTGGACGTATAGGGTG
TGACGCCTGCCCGGTGCCGGAAGGTTAATTGAAGATGTGCAAGCATCGGATCGAAGCCCCG
GTAAACGGCGGCCGTAACTATAACGGTCCTAAGGTAGCGAAATTCCTTGTCGGGTAAGTTCC
GACCCGCACGAATGGCGTAACGATGGCCACACTGTCTCCTCCCGAGACTCAGCGAAGTTGA
AGTGGTTGTGAAGATGCAATCTACCCGCTGCTAGACGGAAAGACCCCGTGAACCTTTACTGT
AGCTTTGCATTGGACTTTGAAGTCACTTGTGTAGGATAGGTGGGAGGCTTGGAAGCAGAGAC
GCCAGTCTCTGTGGAGTCGTCCTTGAAATACCACCCTGGTGTCTTTGAGGTTCTAACCCAGAC
CCGTCATCCGGGTCGGGGACCGTGCATGGTAGGCAGTTTGACTGGGGCGGTCTCCTCCCAAA
GCGTAACGGAGGAGTTCGAAGGTTACCTAGGTCCGGTCGGAAATCGGACTGATAGTGCAAT
GGCAAAAGGTAGCTTAACTGCGAGACCGACAAGTCGGGCAGGTGCGAAAGCAGGACATAGT
GATCCGGTGGTTCTGTATGGAAGGGCCATCGCTCAACGGATAAAAGGTACTCCGGGGATAA
CAGGCTGATTCCGCCCAAGAGTTCATATCGACGGCGGAGTTTGGCACCTCGATGTCGGCTCA
TCACATCCTGGGGCTGTAGTCGGTCCCAAGGGTATGGCTGTTCGCCATTTAAAGTGGTACGT
GAGCTGGGTTTAAAACGTCGTGAGACAGTTTGGTCCCTATCTGCAGTGGGCGTTGGAAGTTT
GACGGGGGCTGCTCCTAGTACGAGAGGACCGGAGTGGACGAACCTCTGGTGTACCGGTTGT
AACGCCAGTTGCATAGCCGGGTAGCTAAGTTCGGAAGAGATAAGCGCTGAAAGCATCTAAG
CGCGAAACTCGCCTGAAGATGAGACTTCCCTTGCGGTTTAACCGCACTAAAGGGTCGTTCGA
GACCAGGACGTTGATAGGTGGGGTGTGGAAGCGCGGTAACGCGTGAAGCTAACCCATACTA
ATTGCCCGTGAGGCTTGACTCT

cDNA: SEQ ID NO: 8

AGAGTCAAGCCTCACGGGCAATTAGTATGGGTTAGCTTCACGCGTTACCGCGCTTCCACACC
CCACCTATCAACGTCCTGGTCTCGAACGACCCTTTAGTGCGGTTAAACCGCAAGGGAAGTCT
CATCTTCAGGCGAGTTTCGCGCTTAGATGCTTTCAGCGCTTATCTCTTCCGAACTTAGCTACC
CGGCTATGCAACTGGCGTTACAACCGGTACACCAGAGGTTCGTCCACTCCGGTCCTCTCGTA
CTAGGAGCAGCCCCCGTCAAACTTCCAACGCCCACTGCAGATAGGGACCAAACTGTCTCACG
ACGTTTTAAACCCAGCTCACGTACCACTTTAAATGGCGAACAGCCATACCCTTGGGACCGAC
TACAGCCCCAGGATGTGATGAGCCGACATCGAGGTGCCAAACTCCGCCGTCGATATGAACTC
TTGGGCGGAATCAGCCTGTTATCCCCGGAGTACCTTTTATCCGTTGAGCGATGGCCCTTCCAT
ACAGAACCACCGGATCACTATGTCCTGCTTTCGCACCTGCCCGACTTGTCGGTCTCGCAGTTA
AGCTACCTTTTGCCATTGCACTATCAGTCCGATTTCCGACCGGACCTAGGTAACCTTCGAACT
CCTCCGTTACGCTTTGGGAGGAGACCGCCCCAGTCAAACTGCCTACCATGCACGGTCCCCGA
CCCGGATGACGGGTCTGGGTTAGAACCTCAAAGACACCAGGGTGGTATTTCAAGGACGACT
CCACAGAGACTGGCGTCTCTGCTTCCAAGCCTCCCACCTATCCTACACAAGTGACTTCAAAG
TCCAATGCAAAGCTACAGTAAAGGTTCACGGGGTCTTTCCGTCTAGCAGCGGGTAGATTGCA
TCTTCACAACCACTTCAACTTCGCTGAGTCTCGGGAGGAGACAGTGTGGCCATCGTTACGCC
ATTCGTGCGGGTCGGAACTTACCCGACAAGGAATTTCGCTACCTTAGGACCGTTATAGTTAC
GGCCGCCGTTTACCGGGGCTTCGATCCGATGCTTGCACATCTTCAATTAACCTTCCGGCACCG
GGCAGGCGTCACACCCTATACGTCCACTTTCGTGTTGGCAGAGTGCTGTGTTTTTAATAAAC
AGTCGCAGCCACCTATTCTCTGCGACCCTCCGGGGCTTACGGAGCAAGTCCTTAACCTTAGA
GGGCATACCTTCTCCCGAAGTTACGGTATCAATTTGCCGAGTTCCTTCTCCCGAGTTCTCTCA
AGCGCCTTAGAATTCTCATCCTGCCCACCTGTGTCGGTTTGCGGTACGGTTCGATTCAAACTG
AAGCTTAGTGGCTTTTCCTGGAAGCGTGGTATCGGTTGCTTCGTGTCCGTAGACACTCGTCAT
CACTTCTCGGTGTTAAGAAAACCCGGATTTGCCTAAGTCTTCCACCTACCGGCTTAAACAAG
CTATTCCAACAGCTTGCCAACCTAACCTTCTCCGTCCCCACATCGCATTTGAATCAAGTACAG
GAATATTAACCTGTTTCCCATCGACTACGCATTTCTGCCTCGCCTTAGGGGCCGACTCACCCT
ACGCCGATGAACGTTGCGTAGGAAACCTTGGGCTTTGCGGCGAGCGGGCTTTTCACCCGCTT
TATCGCTACTCATGTCAACATTCGCACTTCTGATACCTCCAGCACACTTTACAATGCACCTTC
ATCAGCCTACAGAACGCTCCCCTACCATGCCGGTAAACCGGCATACGCAGCTTGGGTTATAG
ATTTGAGCCCCGTTACATCTTCCGCGCAGGACGACTCGACCAGTGAGCTATTACGCTTTCTTT
AAATGATGGCTGCTTCTAAGCCAACATCCTGGCTGTCTGTGCCTTCCCACTTCGTTTACCACT
TAATCTATCATTTGGGACCTTAGCCGGCGGTCTGGGTTGTTTCCCTCTTGACAACGGACGTTA
GCACCCGCTGTCTGTCTCCCGAGGAACCACTTGATGGTATTCTGAGTTTGCCATGGGTTGGTA
AGTTGCAATAACCCCCTAGCCATAACAGTGCTTTACCCCCATCAGTGTCTTGCTCGAGGCAC
TACCTAAATAGTTTTCGGGGAGAACCAGCTATCTCCGAGTTTGTTTAGCCTTTCACCCCTACC
CACAGCTCATCCCCGCATTTTGCAACATGCGTGGGTTCGGTCCTCCAGTACCTGTTACGGCAC
CTTCAACCTGGCCATGGATAGATCACTCGGTTTCGGGTCTACACCCAGCAACTCATCGCCCT
ATTAAGACTCGGTTTCCCTACGCCTCCCCTATCCGGTTAAGCTCGCTACTGAATGTAAGTCGT
TGACCCATTATACAAAAGGTACGCAGTCACACCACTAGGGCGCTCCCACTGTTTGTATGCAT
CAGGTTTCAGGTTCTGTTTCACTCCCCTCCCGGGGTTCTTTTCGCCTTTCCCTCACGGTACTGG
TTCACTATCGGTCGATGATGAGTATTTAGCCTTGGAGGATGGTCCCCCCATATTCAGACAGG
ATTTCACGTGTCCCGCCCTACTTTTCGTACGCTTAGTACCGCTGTTGAGATTTCGAATACGGG
ACTGTCACCCGCTATGGTCAAGCTTCCCAGCTTGTTCTTCTACCTCGACAGTTATTACGTACA
GGCTCCTCCGCGTTCGCTCGCCACTACTTGCGGAATCTCGGTTGATTTCTTTTCCTCCGGGTA
CTTAGATGGTTCAGTTCTCCGGGTTCGCTTCTCTAAGCCTATGTATTCAACTTAGGATACTGC
ACAGAATGCAGTGGGTTTCCCCATTCGGACACCGCGGGATCATTGCTTTATTGCCAGCTCCC
CCGCGCTTTTCGCAGGCTTACACGTCCTTCGTCGCCTATCATCGCCAAGGCATCCGCCTGATG
CACTTATTCACTTGACTCTATCATTTCA

RNA: SEQ ID NO: 9

UGAAAUGAUAGAGUCAAGUGAAUAAGUGCAUCAGGCGGAUGCCUUGGCGAUGAUAGGCG
ACGAAGGACGUGUAAGCCUGCGAAAAGCGCGGGGGAGCUGGCAAUAAAGCAAUGAUCCC
GCGGUGUCCGAAUGGGGAAACCCACUGCAUUCUGUGCAGUAUCCUAAGUUGAAUACAUA
GGCUUAGAGAAGCGAACCCGGAGAACUGAACCAUCUAAGUACCCGGAGGAAAAGAAAUC
AACCGAGAUUCCGCAAGUAGUGGCGAGCGAACGCGGAGGAGCCUGUACGUAAUAACUGU
CGAGGUAGAAGAACAAGCUGGGAAGCUUGACCAUAGCGGGUGACAGUCCCGUAUUCGAA
AUCUCAACAGCGGUACUAAGCGUACGAAAAGUAGGGCGGGACACGUGAAAUCCUGUCUG
AAUAUGGGGGGACCAUCCUCCAAGGCUAAAUACUCAUCAUCGACCGAUAGUGAACCAGUA
CCGUGAGGGAAAGGCGAAAAGAACCCCGGGAGGGGAGUGAAACAGAACCUGAAACCUGA
UGCAUACAAACAGUGGGAGCGCCCUAGUGGUGUGACUGCGUACCUUUUGUAUAAUGGGU
CAACGACUUACAUUCAGUAGCGAGCUUAACCGGAUAGGGGAGGCGUAGGGAAACCGAGU
CUUAAUAGGGCGAUGAGUUGCUGGGUGUAGACCCGAAACCGAGUGAUCUAUCCAUGGCC
AGGUUGAAGGUGCCGUAACAGGUACUGGAGGACCGAACCCACGCAUGUUGCAAAAUGCG
GGGAUGAGCUGUGGGUAGGGGUGAAAGGCUAAACAAACUCGGAGAUAGCUGGUUCUCCC
CGAAAACUAUUUAGGUAGUGCCUCGAGCAAGACACUGAUGGGGGUAAAGCACUGUUAUG
GCUAGGGGGUUAUUGCAACUUACCAACCCAUGGCAAACUCAGAAUACCAUCAAGUGGUUC
CUCGGGAGACAGACAGCGGGUGCUAACGUCCGUUGUCAAGAGGGAAACAACCCAGACCGC
CGGCUAAGGUCCCAAAUGAUAGAUUAAGUGGUAAACGAAGUGGGAAGGCACAGACAGCC
AGGAUGUUGGCUUAGAAGCAGCCAUCAUUUAAAGAAAGCGUAAUAGCUCACUGGUCGAG
UCGUCCUGCGCGGAAGAUGUAACGGGGCUCAAAUCUAUAACCCAAGCUGCGUAUGCCGGU
UUACCGGCAUGGUAGGGGAGCGUUCUGUAGGCUGAUGAAGGUGCAUUGUAAAGUGUGCU
GGAGGUAUCAGAAGUGCGAAUGUUGACAUGAGUAGCGAUAAAGCGGGUGAAAAGCCCGC
UCGCCGCAAAGCCCAAGGUUUCCUACGCAACGUUCAUCGGCGUAGGGUGAGUCGGCCCCU
AAGGCGAGGCAGAAAUGCGUAGUCGAUGGGAAACAGGUUAAUAUUCCUGUACUUGAUUC
AAAUGCGAUGUGGGGACGGAGAAGGUUAGGUUGGCAAGCUGUUGGAAUAGCUUGUUUAA
GCCGGUAGGUGGAAGACUUAGGCAAAUCCGGGUUUUCUUAACACCGAGAAGUGAUGACG
AGUGUCUACGGACACGAAGCAACCGAUACCACGCUUCCAGGAAAAGCCACUAAGCUUCAG
UUUGAAUCGAACCGUACCGCAAACCGACACAGGUGGGCAGGAUGAGAAUUCUAAGGCGC
UUGAGAGAACUCGGGAGAAGGAACUCGGCAAAUUGAUACCGUAACUUCGGGAGAAGGUA
UGCCCUCUAAGGUUAAGGACUUGCUCCGUAAGCCCCGGAGGGUCGCAGAGAAUAGGUGGC
UGCGACUGUUUAUUAAAAACACAGCACUCUGCCAACACGAAAGUGGACGUAUAGGGUGU
GACGCCUGCCCGGUGCCGGAAGGUUAAUUGAAGAUGUGCAAGCAUCGGAUCGAAGCCCCG
GUAAACGGCGGCCGUAACUAUAACGGUCCUAAGGUAGCGAAAUUCCUUGUCGGGUAAGU
UCCGACCCGCACGAAUGGCGUAACGAUGGCCACACUGUCUCCUCCCGAGACUCAGCGAAG
UUGAAGUGGUUGUGAAGAUGCAAUCUACCCGCUGCUAGACGGAAAGACCCCGUGAACCU
UUACUGUAGCUUUGCAUUGGACUUUGAAGUCACUUGUGUAGGAUAGGUGGGAGGCUUGG
AAGCAGAGACGCCAGUCUCUGUGGAGUCGUCCUUGAAAUACCACCCUGGUGUCUUUGAGG
UUCUAACCCAGACCCGUCAUCCGGGUCGGGGACCGUGCAUGGUAGGCAGUUUGACUGGGG
CGGUCUCCUCCCAAAGCGUAACGGAGGAGUUCGAAGGUUACCUAGGUCCGGUCGGAAAUC
GGACUGAUAGUGCAAUGGCAAAAGGUAGCUUAACUGCGAGACCGACAAGUCGGGCAGGU
GCGAAAGCAGGACAUAGUGAUCCGGUGGUUCUGUAUGGAAGGGCCAUCGCUCAACGGAU
AAAAGGUACUCCGGGGAUAACAGGCUGAUUCCGCCCAAGAGUUCAUAUCGACGGCGGAG
UUUGGCACCUCGAUGUCGGCUCAUCACAUCCUGGGGCUGUAGUCGGUCCCAAGGGUAUGG
CUGUUCGCCAUUUAAAGUGGUACGUGAGCUGGGUUUAAAACGUCGUGAGACAGUUUGGU
CCCUAUCUGCAGUGGGCGUUGGAAGUUUGACGGGGGCUGCUCCUAGUACGAGAGGACCG
GAGUGGACGAACCUCUGGUGUACCGGUUGUAACGCCAGUUGCAUAGCCGGGUAGCUAAG
UUCGGAAGAGAUAAGCGCUGAAAGCAUCUAAGCGCGAAACUCGCCUGAAGAUGAGACUU
CCCUUGCGGUUUAACCGCACUAAAGGGUCGUUCGAGACCAGGACGUUGAUAGGUGGGGU
GUGGAAGCGCGGUAACGCGUGAAGCUAACCCAUACUAAUUGCCCGUGAGGCUUGACUCU

> A9Y61_RS07190: 16S ribosomal RNA (1 of 4 copies) NZ_CP016017.1:1329306-1330857 - Is on the negative strand DNA (+ strand): SEQ ID NO: 13

TGAACATAAGAGTTTGATCCTGGCTCAGATTGAACGCTGGCGGCATGCTTTACACATGCAAG
TCGGACGGCAGCACAGGGAAGCTTGCTTCTCGGGTGGCGAGTGGCGAACGGGTGAGTAACA
TATCGGAACGTACCGGGTAGCGGGGGATAACTGATCGAAAGATCAGCTAATACCGCATACG
TCTTGAGAGGGAAAGCAGGGGACCTTCGGGCCTTGCGCTATCCGAGCGGCCGATATCTGATT
AGCTGGTTGGCGGGGTAAAGGCCCACCAAGGCGACGATCAGTAGCGGGTCTGAGAGGATGA
TCCGCCACACTGGGACTGAGACACGGCCCAGACTCCTACGGGAGGCAGCAGTGGGGAATTT
TGGACAATGGGCGCAAGCCTGATCCAGCCATGCCGCGTGTCTGAAGAAGGCCTTCGGGTTGT
AAAGGACTTTTGTCAGGGAAGAAAAGGCCGTTGCCAATATCGGCGGCCGATGACGGTACCT
GAAGAATAAGCACCGGCTAACTACGTGCCAGCAGCCGCGGTAATACGTAGGGTGCGAGCGT
TAATCGGAATTACTGGGCGTAAAGCGGGCGCAGACGGTTACTTAAGCAGGATGTGAAATCC
CCGGGCTCAACCCGGGAACTGCGTTCTGAACTGGGTGACTCGAGTGTGTCAGAGGGAGGTG
GAATTCCACGTGTAGCAGTGAAATGCGTAGAGATGTGGAGGAATACCGATGGCGAAGGCAG
CCTCCTGGGATAACACTGACGTTCATGTCCGAAAGCGTGGGTAGCAAACAGGATTAGATACC
CTGGTAGTCCACGCCCTAAACGATGTCAATTAGCTGTTGGGCAACTTGATTGCTTGGTAGCG
TAGCTAACGCGTGAAATTGACCGCCTGGGGAGTACGGTCGCAAGATTAAAACTCAAAGGAA
TTGACGGGGACCCGCACAAGCGGTGGATGATGTGGATTAATTCGATGCAACGCGAAGAACC
TTACCTGGTTTTGACATGTGCGGAATCCTCCGGAGACGGAGGAGTGCCTTCGGGAGCCGTAA
CACAGGTGCTGCATGGCTGTCGTCAGCTCGTGTCGTGAGATGTTGGGTTAAGTCCCGCAACG
AGCGCAACCCTTGTCATTAGTTGCCATCATTCGGTTGGGCACTCTAATGAGACTGCCGGTGA
CAAGCCGGAGGAAGGTGGGGATGACGTCAAGTCCTCATGGCCCTTATGACCAGGGCTTCAC
ACGTCATACAATGGTCGGTACAGAGGGTAGCCAAGCCGCGAGGCGGAGCCAATCTCACAAA
ACCGATCGTAGTCCGGATTGCACTCTGCAACTCGAGTGCATGAAGTCGGAATCGCTAGTAAT
CGCAGGTCAGCATACTGCGGTGAATACGTTCCCGGGTCTTGTACACACCGCCCGTCACACCA
TGGGAGTGGGGGATACCAGAAGTAGGTAGGGTAACCGCAAGGAGTCCGCTTACCACGGTAT
GCTTCATGACTGGGGTGAAGTCGTAACAAGGTAGCCGTAGGGGAACCTGCGGCTGGATCAC
CTCCTTTCTA

cDNA: SEQ ID NO: 14

TAGAAAGGAGGTGATCCAGCCGCAGGTTCCCCTACGGCTACCTTGTTACGACTTCACCCCAG
TCATGAAGCATACCGTGGTAAGCGGACTCCTTGCGGTTACCCTACCTACTTCTGGTATCCCCC
ACTCCCATGGTGTGACGGGCGGTGTGTACAAGACCCGGGAACGTATTCACCGCAGTATGCTG
ACCTGCGATTACTAGCGATTCCGACTTCATGCACTCGAGTTGCAGAGTGCAATCCGGACTAC
GATCGGTTTTGTGAGATTGGCTCCGCCTCGCGGCTTGGCTACCCTCTGTACCGACCATTGTAT
GACGTGTGAAGCCCTGGTCATAAGGGCCATGAGGACTTGACGTCATCCCCACCTTCCTCCGG
CTTGTCACCGGCAGTCTCATTAGAGTGCCCAACCGAATGATGGCAACTAATGACAAGGGTTG
CGCTCGTTGCGGGACTTAACCCAACATCTCACGACACGAGCTGACGACAGCCATGCAGCACC
TGTGTTACGGCTCCCGAAGGCACTCCTCCGTCTCCGGAGGATTCCGCACATGTCAAAACCAG
GTAAGGTTCTTCGCGTTGCATCGAATTAATCCACATCATCCACCGCTTGTGCGGGTCCCCGTC
AATTCCTTTGAGTTTTAATCTTGCGACCGTACTCCCCAGGCGGTCAATTTCACGCGTTAGCTA
CGCTACCAAGCAATCAAGTTGCCCAACAGCTAATTGACATCGTTTAGGGCGTGGACTACCAG
GGTATCTAATCCTGTTTGCTACCCACGCTTTCGGACATGAACGTCAGTGTTATCCCAGGAGG
CTGCCTTCGCCATCGGTATTCCTCCACATCTCTACGCATTTCACTGCTACACGTGGAATTCCA
CCTCCCTCTGACACACTCGAGTCACCCAGTTCAGAACGCAGTTCCCGGGTTGAGCCCGGGGA
TTTCACATCCTGCTTAAGTAACCGTCTGCGCCCGCTTTACGCCCAGTAATTCCGATTAACGCT
CGCACCCTACGTATTACCGCGGCTGCTGGCACGTAGTTAGCCGGTGCTTATTCTTCAGGTACC
GTCATCGGCCGCCGATATTGGCAACGGCCTTTTCTTCCCTGACAAAAGTCCTTTACAACCCG
AAGGCCTTCTTCAGACACGCGGCATGGCTGGATCAGGCTTGCGCCCATTGTCCAAAATTCCC
CACTGCTGCCTCCCGTAGGAGTCTGGGCCGTGTCTCAGTCCCAGTGTGGCGGATCATCCTCTC
AGACCCGCTACTGATCGTCGCCTTGGTGGGCCTTTACCCCGCCAACCAGCTAATCAGATATC
GGCCGCTCGGATAGCGCAAGGCCCGAAGGTCCCCTGCTTTCCCTCTCAAGACGTATGCGGTA
TTAGCTGATCTTTCGATCAGTTATCCCCCGCTACCCGGTACGTTCCGATATGTTACTCACCCG
TTCGCCACTCGCCACCCGAGAAGCAAGCTTCCCTGTGCTGCCGTCCGACTTGCATGTGTAAA
GCATGCCGCCAGCGTTCAATCTGAGCCAGGATCAAACTCTTATGTTCA

RNA: SEQ ID NO: 15

UGAACAUAAGAGUUUGAUCCUGGCUCAGAUUGAACGCUGGCGGCAUGCUUUACACAUGC
AAGUCGGACGGCAGCACAGGGAAGCUUGCUUCUCGGGUGGCGAGUGGCGAACGGGUGAG
UAACAUAUCGGAACGUACCGGGUAGCGGGGGAUAACUGAUCGAAAGAUCAGCUAAUACC
GCAUACGUCUUGAGAGGGAAAGCAGGGGACCUUCGGGCCUUGCGCUAUCCGAGCGGCCGA
UAUCUGAUUAGCUGGUUGGCGGGGUAAAGGCCCACCAAGGCGACGAUCAGUAGCGGGUC
UGAGAGGAUGAUCCGCCACACUGGGACUGAGACACGGCCCAGACUCCUACGGGAGGCAGC
AGUGGGGAAUUUUGGACAAUGGGCGCAAGCCUGAUCCAGCCAUGCCGCGUGUCUGAAGA
AGGCCUUCGGGUUGUAAAGGACUUUUGUCAGGGAAGAAAAGGCCGUUGCCAAUAUCGGC
GGCCGAUGACGGUACCUGAAGAAUAAGCACCGGCUAACUACGUGCCAGCAGCCGCGGUAA
UACGUAGGGUGCGAGCGUUAAUCGGAAUUACUGGGCGUAAAGCGGGCGCAGACGGUUAC
UUAAGCAGGAUGUGAAAUCCCCGGGCUCAACCCGGGAACUGCGUUCUGAACUGGGUGACU
CGAGUGUGUCAGAGGGAGGUGGAAUUCCACGUGUAGCAGUGAAAUGCGUAGAGAUGUGG
AGGAAUACCGAUGGCGAAGGCAGCCUCCUGGGAUAACACUGACGUUCAUGUCCGAAAGCG
UGGGUAGCAAACAGGAUUAGAUACCCUGGUAGUCCACGCCCUAAACGAUGUCAAUUAGC
UGUUGGGCAACUUGAUUGCUUGGUAGCGUAGCUAACGCGUGAAAUUGACCGCCUGGGGA
GUACGGUCGCAAGAUUAAAACUCAAAGGAAUUGACGGGGACCCGCACAAGCGGUGGAUG
AUGUGGAUUAAUUCGAUGCAACGCGAAGAACCUUACCUGGUUUUGACAUGUGCGGAAUC
CUCCGGAGACGGAGGAGUGCCUUCGGGAGCCGUAACACAGGUGCUGCAUGGCUGUCGUCA
GCUCGUGUCGUGAGAUGUUGGGUUAAGUCCCGCAACGAGCGCAACCCUUGUCAUUAGUU
GCCAUCAUUCGGUUGGGCACUCUAAUGAGACUGCCGGUGACAAGCCGGAGGAAGGUGGG
GAUGACGUCAAGUCCUCAUGGCCCUUAUGACCAGGGCUUCACACGUCAUACAAUGGUCGG
UACAGAGGGUAGCCAAGCCGCGAGGCGGAGCCAAUCUCACAAAACCGAUCGUAGUCCGGA
UUGCACUCUGCAACUCGAGUGCAUGAAGUCGGAAUCGCUAGUAAUCGCAGGUCAGCAUAC
UGCGGUGAAUACGUUCCCGGGUCUUGUACACACCGCCCGUCACACCAUGGGAGUGGGGGA
UACCAGAAGUAGGUAGGGUAACCGCAAGGAGUCCGCUUACCACGGUAUGCUUCAUGACU
GGGGUGAAGUCGUAACAAGGUAGCCGUAGGGGAACCUGCGGCUGGAUCACCUCCUUUCU
A

> A9Y61_09315: 23S ribosomal RNA (1 of 4 copies) NZ_CP016017.1:1718894-1721792 - Is on the negative strand DNA (- strand): SEQ ID NO: 16

TGAAATGATAGAGTCAAGTGAATAAGTGCATCAGGCGGATGCCTTGGCGATGATAGGCGAC
GAAGGACGTGTAAGCCTGCGAAAAGCGCGGGGGAGCTGGCAATAAAGCAATGATCCCGCG
GTGTCCGAATGGGGAAACCCACTGCATTCTGTGCAGTATCCTAAGTTGAATACATAGGCTTA
GAGAAGCGAACCCGGAGAACTGAACCATCTAAGTACCCGGAGGAAAAGAAATCAACCGAG
ATTCCGCAAGTAGTGGCGAGCGAACGCGGAGGAGCCTGTACGTAATAACTGTCGAGGTAGA
AGAACAAGCTGGGAAGCTTGACCATAGCGGGTGACAGTCCCGTATTCGAAATCTCAACAGC
GGTACTAAGCGTACGAAAAGTAGGGCGGGACACGTGAAATCCTGTCTGAATATGGGGGGAC
CATCCTCCAAGGCTAAATACTCATCATCGACCGATAGTGAACCAGTACCGTGAGGGAAAGG
CGAAAAGAACCCCGGGAGGGGAGTGAAACAGAACCTGAAACCTGATGCATACAAACAGTG
GGAGCGCCCTAGTGGTGTGACTGCGTACCTTTTGTATAATGGGTCAACGACTTACATTCAGT
AGCGAGCTTAACCGGATAGGGGAGGCGTAGGGAAACCGAGTCTTAATAGGGCGATGAGTTG
CTGGGTGTAGACCCGAAACCGAGTGATCTATCCATGGCCAGGTTGAAGGTGCCGTAACAGG
TACTGGAGGACCGAACCCACGCATGTTGCAAAATGCGGGGATGAGCTGTGGGTAGGGGTGA
AAGGCTAAACAAACTCGGAGATAGCTGGTTCTCCCCGAAAACTATTTAGGTAGTGCCTCGAG
CAAGACACTGATGGGGGTAAAGCACTGTTATGGCTAGGGGGTTATTGCAACTTACCAACCCA
TGGCAAACTCAGAATACCATCAAGTGGTTCCTCGGGAGACAGACAGCGGGTGCTAACGTCC
GTTGTCAAGAGGGAAACAACCCAGACCGCCGGCTAAGGTCCCAAATGATAGATTAAGTGGT
AAACGAAGTGGGAAGGCACAGACAGCCAGGATGTTGGCTTAGAAGCAGCCATCATTTAAAG
AAAGCGTAATAGCTCACTGGTCGAGTCGTCCTGCGCGGAAGATGTAACGGGGCTCAAATCT
ATAACCGAAGCTGCGGATGCCGGTTTACCGGCATGGTAGGGGAGCGTTCTGTAGGCTGATG
AAGGTGCATTGTAAAGTGTGCTGGAGGTATCAGAAGTGCGAATGTTGACATGAGTAGCGAT
AAAGCGGGTGAAAAGCCCGCTCGCCGAAAGCCCAAGGTTTCCTACGCAACGTTCATCGGCG
TAGGGTGAGTCGGCCCCTAAGGCGAGGCAGAAATGCGTAGTCGATGGGAAACAGGTTAATA
TTCCTGTACTTGATTCAAATGCGATGTGGGGACGGAGAAGGTTAGGTTGGCAAGCTGTTGGA
ATAGCTTGTTTAAGCCGGTAGGTGGAAGACTTAGGCAAATCCGGGTTTTCTTAACACCGAGA
AGTGATGACGAGTGTCTACGGACACGAAGCAACCGATACCACGCTTCCAGGAAAAGCCACT
AAGCTTCAGTTTGAATCGAACCGTACCGCAAACCGACACAGGTGGGCAGGATGAGAATTCT
AAGGCGCTTGAGAGAACTCGGGAGAAGGAACTCGGCAAATTGATACCGTAACTTCGGGAGA
AGGTATGCCCTCTAAGGTTAAGGACTTGCTCCGTAAGCCCCGGAGGGTCGCAGAGAATAGG
TGGCTGCGACTGTTTATTAAAAACACAGCACTCTGCCAACACGAAAGTGGACGTATAGGGTG
TGACGCCTGCCCGGTGCCGGAAGGTTAATTGAAGATGTGCAAGCATCGGATCGAAGCCCCG
GTAAACGGCGGCCGTAACTATAACGGTCCTAAGGTAGCGAAATTCCTTGTCGGGTAAGTTCC
GACCCGCACGAATGGCGTAACGATGGCCACACTGTCTCCTCCCGAGACTCAGCGAAGTTGA
AGTGGTTGTGAAGATGCAATCTACCCGCTGCTAGACGGAAAGACCCCGTGAACCTTTACTGT
AGCTTTGCATTGGACTTTGAAGTCACTTGTGTAGGATAGGTGGGAGGCTTGGAAGCAGAGAC
GCCAGTCTCTGTGGAGTCGTCCTTGAAATACCACCCTGGTGTCTTTGAGGTTCTAACCCAGAC
CCGTCATCCGGGTCGGGGACCGTGCATGGTAGGCAGTTTGACTGGGGCGGTCTCCTCCCAAA
GCGTAACGGAGGAGTTCGAAGGTTACCTAGGTCCGGTCGGAAATCGGACTGATAGTGCAAT
GGCAAAAGGTAGCTTAACTGCGAGACCGACAAGTCGGGCAGGTGCGAAAGCAGGACATAGT
GATCCGGTGGTTCTGTATGGAAGGGCCATCGCTCAACGGATAAAAGGTACTCCGGGGATAA
CAGGCTGATTCCGCCCAAGAGTTCATATCGACGGCGGAGTTTGGCACCTCGATGTCGGCTCA
TCACATCCTGGGGCTGTAGTCGGTCCCAAGGGTATGGCTGTTCGCCATTTAAAGTGGTACGT
GAGCTGGGTTTAAAACGTCGTGAGACAGTTTGGTCCCTATCTGCAGTGGGCGTTGGAAGTTT
GACGGGGGCTGCTCCTAGTACGAGAGGACCGGAGTGGACGAACCTCTGGTGTACCGGTTGT
AACGCCAGTTGCATAGCCGGGTAGCTAAGTTCGGAAGAGATAAGCGCTGAAAGCATCTAAG
CGCGAAACTCGCCTGAAGATGAGACTTCCCTTGCGGTTTAACCGCACTAAAGGGTCGTTCGA
GACCAGGACGTTGATAGGTGGGGTGTGGAAGCGCGGTAACGCGTGAAGCTAACCCATACTA
ATTGCCCGTGAGGCTTGACTCT

cDNA: SEQ ID NO: 17

AGAGTCAAGCCTCACGGGCAATTAGTATGGGTTAGCTTCACGCGTTACCGCGCTTCCACACC
CCACCTATCAACGTCCTGGTCTCGAACGACCCTTTAGTGCGGTTAAACCGCAAGGGAAGTCT
CATCTTCAGGCGAGTTTCGCGCTTAGATGCTTTCAGCGCTTATCTCTTCCGAACTTAGCTACC
CGGCTATGCAACTGGCGTTACAACCGGTACACCAGAGGTTCGTCCACTCCGGTCCTCTCGTA
CTAGGAGCAGCCCCCGTCAAACTTCCAACGCCCACTGCAGATAGGGACCAAACTGTCTCACG
ACGTTTTAAACCCAGCTCACGTACCACTTTAAATGGCGAACAGCCATACCCTTGGGACCGAC
TACAGCCCCAGGATGTGATGAGCCGACATCGAGGTGCCAAACTCCGCCGTCGATATGAACTC
TTGGGCGGAATCAGCCTGTTATCCCCGGAGTACCTTTTATCCGTTGAGCGATGGCCCTTCCAT
ACAGAACCACCGGATCACTATGTCCTGCTTTCGCACCTGCCCGACTTGTCGGTCTCGCAGTTA
AGCTACCTTTTGCCATTGCACTATCAGTCCGATTTCCGACCGGACCTAGGTAACCTTCGAACT
CCTCCGTTACGCTTTGGGAGGAGACCGCCCCAGTCAAACTGCCTACCATGCACGGTCCCCGA
CCCGGATGACGGGTCTGGGTTAGAACCTCAAAGACACCAGGGTGGTATTTCAAGGACGACT
CCACAGAGACTGGCGTCTCTGCTTCCAAGCCTCCCACCTATCCTACACAAGTGACTTCAAAG
TCCAATGCAAAGCTACAGTAAAGGTTCACGGGGTCTTTCCGTCTAGCAGCGGGTAGATTGCA
TCTTCACAACCACTTCAACTTCGCTGAGTCTCGGGAGGAGACAGTGTGGCCATCGTTACGCC
ATTCGTGCGGGTCGGAACTTACCCGACAAGGAATTTCGCTACCTTAGGACCGTTATAGTTAC
GGCCGCCGTTTACCGGGGCTTCGATCCGATGCTTGCACATCTTCAATTAACCTTCCGGCACCG
GGCAGGCGTCACACCCTATACGTCCACTTTCGTGTTGGCAGAGTGCTGTGTTTTTAATAAAC
AGTCGCAGCCACCTATTCTCTGCGACCCTCCGGGGCTTACGGAGCAAGTCCTTAACCTTAGA
GGGCATACCTTCTCCCGAAGTTACGGTATCAATTTGCCGAGTTCCTTCTCCCGAGTTCTCTCA
AGCGCCTTAGAATTCTCATCCTGCCCACCTGTGTCGGTTTGCGGTACGGTTCGATTCAAACTG
AAGCTTAGTGGCTTTTCCTGGAAGCGTGGTATCGGTTGCTTCGTGTCCGTAGACACTCGTCAT
CACTTCTCGGTGTTAAGAAAACCCGGATTTGCCTAAGTCTTCCACCTACCGGCTTAAACAAG
CTATTCCAACAGCTTGCCAACCTAACCTTCTCCGTCCCCACATCGCATTTGAATCAAGTACAG
GAATATTAACCTGTTTCCCATCGACTACGCATTTCTGCCTCGCCTTAGGGGCCGACTCACCCT
ACGCCGATGAACGTTGCGTAGGAAACCTTGGGCTTTCGGCGAGCGGGCTTTTCACCCGCTTT
ATCGCTACTCATGTCAACATTCGCACTTCTGATACCTCCAGCACACTTTACAATGCACCTTCA
TCAGCCTACAGAACGCTCCCCTACCATGCCGGTAAACCGGCATCCGCAGCTTCGGTTATAGA
TTTGAGCCCCGTTACATCTTCCGCGCAGGACGACTCGACCAGTGAGCTATTACGCTTTCTTTA
AATGATGGCTGCTTCTAAGCCAACATCCTGGCTGTCTGTGCCTTCCCACTTCGTTTACCACTT
AATCTATCATTTGGGACCTTAGCCGGCGGTCTGGGTTGTTTCCCTCTTGACAACGGACGTTAG
CACCCGCTGTCTGTCTCCCGAGGAACCACTTGATGGTATTCTGAGTTTGCCATGGGTTGGTAA
GTTGCAATAACCCCCTAGCCATAACAGTGCTTTACCCCCATCAGTGTCTTGCTCGAGGCACT
ACCTAAATAGTTTTCGGGGAGAACCAGCTATCTCCGAGTTTGTTTAGCCTTTCACCCCTACCC
ACAGCTCATCCCCGCATTTTGCAACATGCGTGGGTTCGGTCCTCCAGTACCTGTTACGGCACC
TTCAACCTGGCCATGGATAGATCACTCGGTTTCGGGTCTACACCCAGCAACTCATCGCCCTA
TTAAGACTCGGTTTCCCTACGCCTCCCCTATCCGGTTAAGCTCGCTACTGAATGTAAGTCGTT
GACCCATTATACAAAAGGTACGCAGTCACACCACTAGGGCGCTCCCACTGTTTGTATGCATC
AGGTTTCAGGTTCTGTTTCACTCCCCTCCCGGGGTTCTTTTCGCCTTTCCCTCACGGTACTGGT
TCACTATCGGTCGATGATGAGTATTTAGCCTTGGAGGATGGTCCCCCCATATTCAGACAGGA
TTTCACGTGTCCCGCCCTACTTTTCGTACGCTTAGTACCGCTGTTGAGATTTCGAATACGGGA
CTGTCACCCGCTATGGTCAAGCTTCCCAGCTTGTTCTTCTACCTCGACAGTTATTACGTACAG
GCTCCTCCGCGTTCGCTCGCCACTACTTGCGGAATCTCGGTTGATTTCTTTTCCTCCGGGTAC
TTAGATGGTTCAGTTCTCCGGGTTCGCTTCTCTAAGCCTATGTATTCAACTTAGGATACTGCA
CAGAATGCAGTGGGTTTCCCCATTCGGACACCGCGGGATCATTGCTTTATTGCCAGCTCCCC
CGCGCTTTTCGCAGGCTTACACGTCCTTCGTCGCCTATCATCGCCAAGGCATCCGCCTGATGC
ACTTATTCACTTGACTCTATCATTTCA

RNA: SEQ ID NO: 18

UGAAAUGAUAGAGUCAAGUGAAUAAGUGCAUCAGGCGGAUGCCUUGGCGAUGAUAGGCG
ACGAAGGACGUGUAAGCCUGCGAAAAGCGCGGGGGAGCUGGCAAUAAAGCAAUGAUCCC
GCGGUGUCCGAAUGGGGAAACCCACUGCAUUCUGUGCAGUAUCCUAAGUUGAAUACAUA
GGCUUAGAGAAGCGAACCCGGAGAACUGAACCAUCUAAGUACCCGGAGGAAAAGAAAUC
AACCGAGAUUCCGCAAGUAGUGGCGAGCGAACGCGGAGGAGCCUGUACGUAAUAACUGU
CGAGGUAGAAGAACAAGCUGGGAAGCUUGACCAUAGCGGGUGACAGUCCCGUAUUCGAA
AUCUCAACAGCGGUACUAAGCGUACGAAAAGUAGGGCGGGACACGUGAAAUCCUGUCUG
AAUAUGGGGGGACCAUCCUCCAAGGCUAAAUACUCAUCAUCGACCGAUAGUGAACCAGUA
CCGUGAGGGAAAGGCGAAAAGAACCCCGGGAGGGGAGUGAAACAGAACCUGAAACCUGA
UGCAUACAAACAGUGGGAGCGCCCUAGUGGUGUGACUGCGUACCUUUUGUAUAAUGGGU
CAACGACUUACAUUCAGUAGCGAGCUUAACCGGAUAGGGGAGGCGUAGGGAAACCGAGU
CUUAAUAGGGCGAUGAGUUGCUGGGUGUAGACCCGAAACCGAGUGAUCUAUCCAUGGCC
AGGUUGAAGGUGCCGUAACAGGUACUGGAGGACCGAACCCACGCAUGUUGCAAAAUGCG
GGGAUGAGCUGUGGGUAGGGGUGAAAGGCUAAACAAACUCGGAGAUAGCUGGUUCUCCC
CGAAAACUAUUUAGGUAGUGCCUCGAGCAAGACACUGAUGGGGGUAAAGCACUGUUAUG
GCUAGGGGGUUAUUGCAACUUACCAACCCAUGGCAAACUCAGAAUACCAUCAAGUGGUUC
CUCGGGAGACAGACAGCGGGUGCUAACGUCCGUUGUCAAGAGGGAAACAACCCAGACCGC
CGGCUAAGGUCCCAAAUGAUAGAUUAAGUGGUAAACGAAGUGGGAAGGCACAGACAGCC
AGGAUGUUGGCUUAGAAGCAGCCAUCAUUUAAAGAAAGCGUAAUAGCUCACUGGUCGAG
UCGUCCUGCGCGGAAGAUGUAACGGGGCUCAAAUCUAUAACCGAAGCUGCGGAUGCCGGU
UUACCGGCAUGGUAGGGGAGCGUUCUGUAGGCUGAUGAAGGUGCAUUGUAAAGUGUGCU
GGAGGUAUCAGAAGUGCGAAUGUUGACAUGAGUAGCGAUAAAGCGGGUGAAAAGCCCGC
UCGCCGAAAGCCCAAGGUUUCCUACGCAACGUUCAUCGGCGUAGGGUGAGUCGGCCCCUA
AGGCGAGGCAGAAAUGCGUAGUCGAUGGGAAACAGGUUAAUAUUCCUGUACUUGAUUCA
AAUGCGAUGUGGGGACGGAGAAGGUUAGGUUGGCAAGCUGUUGGAAUAGCUUGUUUAAG
CCGGUAGGUGGAAGACUUAGGCAAAUCCGGGUUUUCUUAACACCGAGAAGUGAUGACGA
GUGUCUACGGACACGAAGCAACCGAUACCACGCUUCCAGGAAAAGCCACUAAGCUUCAGU
UUGAAUCGAACCGUACCGCAAACCGACACAGGUGGGCAGGAUGAGAAUUCUAAGGCGCU
UGAGAGAACUCGGGAGAAGGAACUCGGCAAAUUGAUACCGUAACUUCGGGAGAAGGUAU
GCCCUCUAAGGUUAAGGACUUGCUCCGUAAGCCCCGGAGGGUCGCAGAGAAUAGGUGGCU
GCGACUGUUUAUUAAAAACACAGCACUCUGCCAACACGAAAGUGGACGUAUAGGGUGUG
ACGCCUGCCCGGUGCCGGAAGGUUAAUUGAAGAUGUGCAAGCAUCGGAUCGAAGCCCCGG
UAAACGGCGGCCGUAACUAUAACGGUCCUAAGGUAGCGAAAUUCCUUGUCGGGUAAGUU
CCGACCCGCACGAAUGGCGUAACGAUGGCCACACUGUCUCCUCCCGAGACUCAGCGAAGU
UGAAGUGGUUGUGAAGAUGCAAUCUACCCGCUGCUAGACGGAAAGACCCCGUGAACCUU
UACUGUAGCUUUGCAUUGGACUUUGAAGUCACUUGUGUAGGAUAGGUGGGAGGCUUGGA
AGCAGAGACGCCAGUCUCUGUGGAGUCGUCCUUGAAAUACCACCCUGGUGUCUUUGAGGU
UCUAACCCAGACCCGUCAUCCGGGUCGGGGACCGUGCAUGGUAGGCAGUUUGACUGGGGC
GGUCUCCUCCCAAAGCGUAACGGAGGAGUUCGAAGGUUACCUAGGUCCGGUCGGAAAUCG
GACUGAUAGUGCAAUGGCAAAAGGUAGCUUAACUGCGAGACCGACAAGUCGGGCAGGUG
CGAAAGCAGGACAUAGUGAUCCGGUGGUUCUGUAUGGAAGGGCCAUCGCUCAACGGAUA
AAAGGUACUCCGGGGAUAACAGGCUGAUUCCGCCCAAGAGUUCAUAUCGACGGCGGAGU
UUGGCACCUCGAUGUCGGCUCAUCACAUCCUGGGGCUGUAGUCGGUCCCAAGGGUAUGGC
UGUUCGCCAUUUAAAGUGGUACGUGAGCUGGGUUUAAAACGUCGUGAGACAGUUUGGUC
CCUAUCUGCAGUGGGCGUUGGAAGUUUGACGGGGGCUGCUCCUAGUACGAGAGGACCGG
AGUGGACGAACCUCUGGUGUACCGGUUGUAACGCCAGUUGCAUAGCCGGGUAGCUAAGU
UCGGAAGAGAUAAGCGCUGAAAGCAUCUAAGCGCGAAACUCGCCUGAAGAUGAGACUUC
CCUUGCGGUUUAACCGCACUAAAGGGUCGUUCGAGACCAGGACGUUGAUAGGUGGGGUG
UGGAAGCGCGGUAACGCGUGAAGCUAACCCAUACUAAUUGCCCGUGAGGCUUGACUCU

> A9Y61_09330: 16S ribosomal RNA (1 of 4 copies) NZ_CP016017.1:1722390-17239411721792 — Is on the negative strand DNA (- strand): SEQ ID NO: 19

TGAACATAAGAGTTTGATCCTGGCTCAGATTGAACGCTGGCGGCATGCTTTACACATGCAAG
TCGGACGGCAGCACAGGGAAGCTTGCTTCTCGGGTGGCGAGTGGCGAACGGGTGAGTAACA
TATCGGAACGTACCGGGTAGCGGGGGATAACTGATCGAAAGATCAGCTAATACCGCATACG
TCTTGAGAGGGAAAGCAGGGGACCTTCGGGCCTTGCGCTATCCGAGCGGCCGATATCTGATT
AGCTGGTTGGCGGGGTAAAGGCCCACCAAGGCGACGATCAGTAGCGGGTCTGAGAGGATGA
TCCGCCACACTGGGACTGAGACACGGCCCAGACTCCTACGGGAGGCAGCAGTGGGGAATTT
TGGACAATGGGCGCAAGCCTGATCCAGCCATGCCGCGTGTCTGAAGAAGGCCTTCGGGTTGT
AAAGGACTTTTGTCAGGGAAGAAAAGGCCGTTGCCAATATCGGCGGCCGATGACGGTACCT
GAAGAATAAGCACCGGCTAACTACGTGCCAGCAGCCGCGGTAATACGTAGGGTGCGAGCGT
TAATCGGAATTACTGGGCGTAAAGCGGGCGCAGACGGTTACTTAAGCAGGATGTGAAATCC
CCGGGCTCAACCCGGGAACTGCGTTCTGAACTGGGTGACTCGAGTGTGTCAGAGGGAGGTG
GAATTCCACGTGTAGCAGTGAAATGCGTAGAGATGTGGAGGAATACCGATGGCGAAGGCAG
CCTCCTGGGATAACACTGACGTTCATGTCCGAAAGCGTGGGTAGCAAACAGGATTAGATACC
CTGGTAGTCCACGCCCTAAACGATGTCAATTAGCTGTTGGGCAACTTGATTGCTTGGTAGCG
TAGCTAACGCGTGAAATTGACCGCCTGGGGAGTACGGTCGCAAGATTAAAACTCAAAGGAA
TTGACGGGGACCCGCACAAGCGGTGGATGATGTGGATTAATTCGATGCAACGCGAAGAACC
TTACCTGGTTTTGACATGTGCGGAATCCTCCGGAGACGGAGGAGTGCCTTCGGGAGCCGTAA
CACAGGTGCTGCATGGCTGTCGTCAGCTCGTGTCGTGAGATGTTGGGTTAAGTCCCGCAACG
AGCGCAACCCTTGTCATTAGTTGCCATCATTCGGTTGGGCACTCTAATGAGACTGCCGGTGA
CAAGCCGGAGGAAGGTGGGGATGACGTCAAGTCCTCATGGCCCTTATGACCAGGGCTTCAC
ACGTCATACAATGGTCGGTACAGAGGGTAGCCAAGCCGCGAGGCGGAGCCAATCTCACAAA
ACCGATCGTAGTCCGGATTGCACTCTGCAACTCGAGTGCATGAAGTCGGAATCGCTAGTAAT
CGCAGGTCAGCATACTGCGGTGAATACGTTCCCGGGTCTTGTACACACCGCCCGTCACACCA
TGGGAGTGGGGGATACCAGAAGTAGGTAGGGTAACCGCAAGGAGTCCGCTTACCACGGTAT
GCTTCATGACTGGGGTGAAGTCGTAACAAGGTAGCCGTAGGGGAACCTGCGGCTGGATCAC
CTCCTTTCTA

cDNA: SEQ ID NO: 20

TAGAAAGGAGGTGATCCAGCCGCAGGTTCCCCTACGGCTACCTTGTTACGACTTCACCCCAG
TCATGAAGCATACCGTGGTAAGCGGACTCCTTGCGGTTACCCTACCTACTTCTGGTATCCCCC
ACTCCCATGGTGTGACGGGCGGTGTGTACAAGACCCGGGAACGTATTCACCGCAGTATGCTG
ACCTGCGATTACTAGCGATTCCGACTTCATGCACTCGAGTTGCAGAGTGCAATCCGGACTAC
GATCGGTTTTGTGAGATTGGCTCCGCCTCGCGGCTTGGCTACCCTCTGTACCGACCATTGTAT
GACGTGTGAAGCCCTGGTCATAAGGGCCATGAGGACTTGACGTCATCCCCACCTTCCTCCGG
CTTGTCACCGGCAGTCTCATTAGAGTGCCCAACCGAATGATGGCAACTAATGACAAGGGTTG
CGCTCGTTGCGGGACTTAACCCAACATCTCACGACACGAGCTGACGACAGCCATGCAGCACC
TGTGTTACGGCTCCCGAAGGCACTCCTCCGTCTCCGGAGGATTCCGCACATGTCAAAACCAG
GTAAGGTTCTTCGCGTTGCATCGAATTAATCCACATCATCCACCGCTTGTGCGGGTCCCCGTC
AATTCCTTTGAGTTTTAATCTTGCGACCGTACTCCCCAGGCGGTCAATTTCACGCGTTAGCTA
CGCTACCAAGCAATCAAGTTGCCCAACAGCTAATTGACATCGTTTAGGGCGTGGACTACCAG
GGTATCTAATCCTGTTTGCTACCCACGCTTTCGGACATGAACGTCAGTGTTATCCCAGGAGG
CTGCCTTCGCCATCGGTATTCCTCCACATCTCTACGCATTTCACTGCTACACGTGGAATTCCA
CCTCCCTCTGACACACTCGAGTCACCCAGTTCAGAACGCAGTTCCCGGGTTGAGCCCGGGGA
TTTCACATCCTGCTTAAGTAACCGTCTGCGCCCGCTTTACGCCCAGTAATTCCGATTAACGCT
CGCACCCTACGTATTACCGCGGCTGCTGGCACGTAGTTAGCCGGTGCTTATTCTTCAGGTACC
GTCATCGGCCGCCGATATTGGCAACGGCCTTTTCTTCCCTGACAAAAGTCCTTTACAACCCG
AAGGCCTTCTTCAGACACGCGGCATGGCTGGATCAGGCTTGCGCCCATTGTCCAAAATTCCC
CACTGCTGCCTCCCGTAGGAGTCTGGGCCGTGTCTCAGTCCCAGTGTGGCGGATCATCCTCTC
AGACCCGCTACTGATCGTCGCCTTGGTGGGCCTTTACCCCGCCAACCAGCTAATCAGATATC
GGCCGCTCGGATAGCGCAAGGCCCGAAGGTCCCCTGCTTTCCCTCTCAAGACGTATGCGGTA
TTAGCTGATCTTTCGATCAGTTATCCCCCGCTACCCGGTACGTTCCGATATGTTACTCACCCG
TTCGCCACTCGCCACCCGAGAAGCAAGCTTCCCTGTGCTGCCGTCCGACTTGCATGTGTAAA
GCATGCCGCCAGCGTTCAATCTGAGCCAGGATCAAACTCTTATGTTCA

RNA: SEQ ID NO: 21

UGAACAUAAGAGUUUGAUCCUGGCUCAGAUUGAACGCUGGCGGCAUGCUUUACACAUGC
AAGUCGGACGGCAGCACAGGGAAGCUUGCUUCUCGGGUGGCGAGUGGCGAACGGGUGAG
UAACAUAUCGGAACGUACCGGGUAGCGGGGGAUAACUGAUCGAAAGAUCAGCUAAUACC
GCAUACGUCUUGAGAGGGAAAGCAGGGGACCUUCGGGCCUUGCGCUAUCCGAGCGGCCGA
UAUCUGAUUAGCUGGUUGGCGGGGUAAAGGCCCACCAAGGCGACGAUCAGUAGCGGGUC
UGAGAGGAUGAUCCGCCACACUGGGACUGAGACACGGCCCAGACUCCUACGGGAGGCAGC
AGUGGGGAAUUUUGGACAAUGGGCGCAAGCCUGAUCCAGCCAUGCCGCGUGUCUGAAGA
AGGCCUUCGGGUUGUAAAGGACUUUUGUCAGGGAAGAAAAGGCCGUUGCCAAUAUCGGC
GGCCGAUGACGGUACCUGAAGAAUAAGCACCGGCUAACUACGUGCCAGCAGCCGCGGUAA
UACGUAGGGUGCGAGCGUUAAUCGGAAUUACUGGGCGUAAAGCGGGCGCAGACGGUUAC
UUAAGCAGGAUGUGAAAUCCCCGGGCUCAACCCGGGAACUGCGUUCUGAACUGGGUGACU
CGAGUGUGUCAGAGGGAGGUGGAAUUCCACGUGUAGCAGUGAAAUGCGUAGAGAUGUGG
AGGAAUACCGAUGGCGAAGGCAGCCUCCUGGGAUAACACUGACGUUCAUGUCCGAAAGCG
UGGGUAGCAAACAGGAUUAGAUACCCUGGUAGUCCACGCCCUAAACGAUGUCAAUUAGC
UGUUGGGCAACUUGAUUGCUUGGUAGCGUAGCUAACGCGUGAAAUUGACCGCCUGGGGA
GUACGGUCGCAAGAUUAAAACUCAAAGGAAUUGACGGGGACCCGCACAAGCGGUGGAUG
AUGUGGAUUAAUUCGAUGCAACGCGAAGAACCUUACCUGGUUUUGACAUGUGCGGAAUC
CUCCGGAGACGGAGGAGUGCCUUCGGGAGCCGUAACACAGGUGCUGCAUGGCUGUCGUCA
GCUCGUGUCGUGAGAUGUUGGGUUAAGUCCCGCAACGAGCGCAACCCUUGUCAUUAGUU
GCCAUCAUUCGGUUGGGCACUCUAAUGAGACUGCCGGUGACAAGCCGGAGGAAGGUGGG
GAUGACGUCAAGUCCUCAUGGCCCUUAUGACCAGGGCUUCACACGUCAUACAAUGGUCGG
UACAGAGGGUAGCCAAGCCGCGAGGCGGAGCCAAUCUCACAAAACCGAUCGUAGUCCGGA
UUGCACUCUGCAACUCGAGUGCAUGAAGUCGGAAUCGCUAGUAAUCGCAGGUCAGCAUAC
UGCGGUGAAUACGUUCCCGGGUCUUGUACACACCGCCCGUCACACCAUGGGAGUGGGGGA
UACCAGAAGUAGGUAGGGUAACCGCAAGGAGUCCGCUUACCACGGUAUGCUUCAUGACU
GGGGUGAAGUCGUAACAAGGUAGCCGUAGGGGAACCUGCGGCUGGAUCACCUCCUUUCU
A

> A9Y61_10490: 23S ribosomal RNA (1 of 4 copies) NZ_CP016017.1:1941315-1944213 - Is on the negative strand DNA (- strand): SEQ ID NO: 22

TGAAATGATAGAGTCAAGTGAATAAGTGCATCAGGCGGATGCCTTGGCGATGATAGGCGAC
GAAGGACGTGTAAGCCTGCGAAAAGCGCGGGGGAGCTGGCAATAAAGCAATGATCCCGCG
GTGTCCGAATGGGGAAACCCACTGCATTCTGTGCAGTATCCTAAGTTGAATACATAGGCTTA
GAGAAGCGAACCCGGAGAACTGACCCATCTAAGTACCCGGAGGAAAAGAAATCAACCGAG
ATTCCGCAAGTAGTGGCGAGCGAACGCGGAGGAGCCTGTACGTAATAACTGTCGAGGTAGA
AGAACAAGCTGGGAAGCTTGACCATAGCGGGTGACAGTCCCGTATTCGAAATCTCAACAGC
GGTACTAAGCGTACGAAAAGTAGGGCGGGACACGTGAAATCCTGTCTGAATATGGGGGGAC
CATCCTCCAAGGCTAAATACTCATCATCGACCGATAGTGAACCAGTACCGTGAGGGAAAGG
CGAAAAGAACCCCGGGAGGGGAGTGAAACAGAACCTGAAACCTGATGCATACAAACAGTG
GGAGCGCCCTAGTGGTGTGACTGCGTACCTTTTGTATAATGGGTCAACGACTTACATTCAGT
AGCGAGCTTAACCGGATAGGGGAGGCGTAGGGAAACCGAGTCTTAATAGGGCGATGAGTTG
CTGGGTGTAGACCCGAAACCGAGTGATCTATCCATGGCCAGGTTGAAGGTGCCGTAACAGG
TACTGGAGGACCGAACCCACGCATGTTGCAAAATGCGGGGATGAGCTGTGGGTAGGGGTGA
AAGGCTAAACAAACTCGGAGATAGCTGGTTCTCCCCGAAAACTATTTAGGTAGTGCCTCGAG
CAAGACACTGATGGGGGTAAAGCACTGTTATGGCTAGGGGGTTATTGCAACTTACCAACCCA
TGGCAAACTCAGAATACCATCAAGTGGTTCCTCGGGAGACAGACAGCGGGTGCTAACGTCC
GTTGTCAAGAGGGAAACAACCCAGACCGCCGGCTAAGGTCCCAAATGATAGATTAAGTGGT
AAACGAAGTGGGAAGGCACAGACAGCCAGGATGTTGGCTTAGAAGCAGCCATCATTTAAAG
AAAGCGTAATAGCTCACTGGTCGAGTCGTCCTGCGCGGAAGATGTAACGGGGCTCAAATCT
ATAACCGAAGCTGCGGATGCCGGTTTACCGGCATGGTAGGGGAGCGTTCTGTAGGCTGATG
AAGGTGCATTGTAAAGTGTGCTGGAGGTATCAGAAGTGCGAATGTTGACATGAGTAGCGAT
AAAGCGGGTGAAAAGCCCGCTCGCCGAAAGCCCAAGGTTTCCTACGCAACGTTCATCGGCG
TAGGGTGAGTCGGCCCCTAAGGCGAGGCAGAAATGCGTAGTCGATGGGAAACAGGTTAATA
TTCCTGTACTTGATTCAAATGCGATGTGGGGACGGAGAAGGTTAGGTTGGCAAGCTGTTGGA
ATAGCTTGTTTAAGCCGGTAGGTGGAAGACTTAGGCAAATCCGGGTTTTCTTAACACCGAGA
AGTGATGACGAGTGTCTACGGACACGAAGCAACCGATACCACGCTTCCAGGAAAAGCCACT
AAGCTTCAGTTTGAATCGAACCGTACCGCAAACCGACACAGGTGGGCAGGATGAGAATTCT
AAGGCGCTTGAGAGAACTCGGGAGAAGGAACTCGGCAAATTGATACCGTAACTTCGGGAGA
AGGTATGCCCTCTAAGGTTAAGGACTTGCTCCGTAAGCCCCGGAGGGTCGCAGAGAATAGG
TGGCTGCGACTGTTTATTAAAAACACAGCACTCTGCCAACACGAAAGTGGACGTATAGGGTG
TGACGCCTGCCCGGTGCCGGAAGGTTAATTGAAGATGTGCAAGCATCGGATCGAAGCCCCG
GTAAACGGCGGCCGTAACTATAACGGTCCTAAGGTAGCGAAATTCCTTGTCGGGTAAGTTCC
GACCCGCACGAATGGCGTAACGATGGCCACACTGTCTCCTCCCGAGACTCAGCGAAGTTGA
AGTGGTTGTGAAGATGCAATCTACCCGCTGCTAGACGGAAAGACCCCGTGAACCTTTACTGT
AGCTTTGCATTGGACTTTGAAGTCACTTGTGTAGGATAGGTGGGAGGCTTGGAAGCAGAGAC
GCCAGTCTCTGTGGAGTCGTCCTTGAAATACCACCCTGGTGTCTTTGAGGTTCTAACCCAGAC
CCGTCATCCGGGTCGGGGACCGTGCATGGTAGGCAGTTTGACTGGGGCGGTCTCCTCCCAAA
GCGTAACGGAGGAGTTCGAAGGTTACCTAGGTCCGGTCGGAAATCGGACTGATAGTGCAAT
GGCAAAAGGTAGCTTAACTGCGAGACCGACAAGTCGGGCAGGTGCGAAAGCAGGACATAGT
GATCCGGTGGTTCTGTATGGAAGGGCCATCGCTCAACGGATAAAAGGTACTCCGGGGATAA
CAGGCTGATTCCGCCCAAGAGTTCATATCGACGGCGGAGTTTGGCACCTCGATGTCGGCTCA
TCACATCCTGGGGCTGTAGTCGGTCCCAAGGGTATGGCTGTTCGCCATTTAAAGTGGTACGT
GAGCTGGGTTTAAAACGTCGTGAGACAGTTTGGTCCCTATCTGCAGTGGGCGTTGGAAGTTT
GACGGGGGCTGCTCCTAGTACGAGAGGACCGGAGTGGACGAACCTCTGGTGTACCGGTTGT
AACGCCAGTTGCATAGCCGGGTAGCTAAGTTCGGAAGAGATAAGCGCTGAAAGCATCTAAG
CGCGAAACTCGCCTGAAGATGAGACTTCCCTTGCGGTTTAACCGCACTAAAGGGTCGTTCGA
GACCAGGACGTTGATAGGTGGGGTGTGGAAGCGCGGTAACGCGTGAAGCTAACCCATACTA
ATTGCCCGTGAGGCTTGACTCT

cDNA: SEQ ID NO: 23

AGAGTCAAGCCTCACGGGCAATTAGTATGGGTTAGCTTCACGCGTTACCGCGCTTCCACACC
CCACCTATCAACGTCCTGGTCTCGAACGACCCTTTAGTGCGGTTAAACCGCAAGGGAAGTCT
CATCTTCAGGCGAGTTTCGCGCTTAGATGCTTTCAGCGCTTATCTCTTCCGAACTTAGCTACC
CGGCTATGCAACTGGCGTTACAACCGGTACACCAGAGGTTCGTCCACTCCGGTCCTCTCGTA
CTAGGAGCAGCCCCCGTCAAACTTCCAACGCCCACTGCAGATAGGGACCAAACTGTCTCACG
ACGTTTTAAACCCAGCTCACGTACCACTTTAAATGGCGAACAGCCATACCCTTGGGACCGAC
TACAGCCCCAGGATGTGATGAGCCGACATCGAGGTGCCAAACTCCGCCGTCGATATGAACTC
TTGGGCGGAATCAGCCTGTTATCCCCGGAGTACCTTTTATCCGTTGAGCGATGGCCCTTCCAT
ACAGAACCACCGGATCACTATGTCCTGCTTTCGCACCTGCCCGACTTGTCGGTCTCGCAGTTA
AGCTACCTTTTGCCATTGCACTATCAGTCCGATTTCCGACCGGACCTAGGTAACCTTCGAACT
CCTCCGTTACGCTTTGGGAGGAGACCGCCCCAGTCAAACTGCCTACCATGCACGGTCCCCGA
CCCGGATGACGGGTCTGGGTTAGAACCTCAAAGACACCAGGGTGGTATTTCAAGGACGACT
CCACAGAGACTGGCGTCTCTGCTTCCAAGCCTCCCACCTATCCTACACAAGTGACTTCAAAG
TCCAATGCAAAGCTACAGTAAAGGTTCACGGGGTCTTTCCGTCTAGCAGCGGGTAGATTGCA
TCTTCACAACCACTTCAACTTCGCTGAGTCTCGGGAGGAGACAGTGTGGCCATCGTTACGCC
ATTCGTGCGGGTCGGAACTTACCCGACAAGGAATTTCGCTACCTTAGGACCGTTATAGTTAC
GGCCGCCGTTTACCGGGGCTTCGATCCGATGCTTGCACATCTTCAATTAACCTTCCGGCACCG
GGCAGGCGTCACACCCTATACGTCCACTTTCGTGTTGGCAGAGTGCTGTGTTTTTAATAAAC
AGTCGCAGCCACCTATTCTCTGCGACCCTCCGGGGCTTACGGAGCAAGTCCTTAACCTTAGA
GGGCATACCTTCTCCCGAAGTTACGGTATCAATTTGCCGAGTTCCTTCTCCCGAGTTCTCTCA
AGCGCCTTAGAATTCTCATCCTGCCCACCTGTGTCGGTTTGCGGTACGGTTCGATTCAAACTG
AAGCTTAGTGGCTTTTCCTGGAAGCGTGGTATCGGTTGCTTCGTGTCCGTAGACACTCGTCAT
CACTTCTCGGTGTTAAGAAAACCCGGATTTGCCTAAGTCTTCCACCTACCGGCTTAAACAAG
CTATTCCAACAGCTTGCCAACCTAACCTTCTCCGTCCCCACATCGCATTTGAATCAAGTACAG
GAATATTAACCTGTTTCCCATCGACTACGCATTTCTGCCTCGCCTTAGGGGCCGACTCACCCT
ACGCCGATGAACGTTGCGTAGGAAACCTTGGGCTTTCGGCGAGCGGGCTTTTCACCCGCTTT
ATCGCTACTCATGTCAACATTCGCACTTCTGATACCTCCAGCACACTTTACAATGCACCTTCA
TCAGCCTACAGAACGCTCCCCTACCATGCCGGTAAACCGGCATCCGCAGCTTCGGTTATAGA
TTTGAGCCCCGTTACATCTTCCGCGCAGGACGACTCGACCAGTGAGCTATTACGCTTTCTTTA
AATGATGGCTGCTTCTAAGCCAACATCCTGGCTGTCTGTGCCTTCCCACTTCGTTTACCACTT
AATCTATCATTTGGGACCTTAGCCGGCGGTCTGGGTTGTTTCCCTCTTGACAACGGACGTTAG
CACCCGCTGTCTGTCTCCCGAGGAACCACTTGATGGTATTCTGAGTTTGCCATGGGTTGGTAA
GTTGCAATAACCCCCTAGCCATAACAGTGCTTTACCCCCATCAGTGTCTTGCTCGAGGCACT
ACCTAAATAGTTTTCGGGGAGAACCAGCTATCTCCGAGTTTGTTTAGCCTTTCACCCCTACCC
ACAGCTCATCCCCGCATTTTGCAACATGCGTGGGTTCGGTCCTCCAGTACCTGTTACGGCACC
TTCAACCTGGCCATGGATAGATCACTCGGTTTCGGGTCTACACCCAGCAACTCATCGCCCTA
TTAAGACTCGGTTTCCCTACGCCTCCCCTATCCGGTTAAGCTCGCTACTGAATGTAAGTCGTT
GACCCATTATACAAAAGGTACGCAGTCACACCACTAGGGCGCTCCCACTGTTTGTATGCATC
AGGTTTCAGGTTCTGTTTCACTCCCCTCCCGGGGTTCTTTTCGCCTTTCCCTCACGGTACTGGT
TCACTATCGGTCGATGATGAGTATTTAGCCTTGGAGGATGGTCCCCCCATATTCAGACAGGA
TTTCACGTGTCCCGCCCTACTTTTCGTACGCTTAGTACCGCTGTTGAGATTTCGAATACGGGA
CTGTCACCCGCTATGGTCAAGCTTCCCAGCTTGTTCTTCTACCTCGACAGTTATTACGTACAG
GCTCCTCCGCGTTCGCTCGCCACTACTTGCGGAATCTCGGTTGATTTCTTTTCCTCCGGGTAC
TTAGATGGGTCAGTTCTCCGGGTTCGCTTCTCTAAGCCTATGTATTCAACTTAGGATACTGCA
CAGAATGCAGTGGGTTTCCCCATTCGGACACCGCGGGATCATTGCTTTATTGCCAGCTCCCC
CGCGCTTTTCGCAGGCTTACACGTCCTTCGTCGCCTATCATCGCCAAGGCATCCGCCTGATGC
ACTTATTCACTTGACTCTATCATTTCA

RNA: SEQ ID NO: 24

UGAAAUGAUAGAGUCAAGUGAAUAAGUGCAUCAGGCGGAUGCCUUGGCGAUGAUAGGCG
ACGAAGGACGUGUAAGCCUGCGAAAAGCGCGGGGGAGCUGGCAAUAAAGCAAUGAUCCC
GCGGUGUCCGAAUGGGGAAACCCACUGCAUUCUGUGCAGUAUCCUAAGUUGAAUACAUA
GGCUUAGAGAAGCGAACCCGGAGAACUGACCCAUCUAAGUACCCGGAGGAAAAGAAAUC
AACCGAGAUUCCGCAAGUAGUGGCGAGCGAACGCGGAGGAGCCUGUACGUAAUAACUGU
CGAGGUAGAAGAACAAGCUGGGAAGCUUGACCAUAGCGGGUGACAGUCCCGUAUUCGAA
AUCUCAACAGCGGUACUAAGCGUACGAAAAGUAGGGCGGGACACGUGAAAUCCUGUCUG
AAUAUGGGGGGACCAUCCUCCAAGGCUAAAUACUCAUCAUCGACCGAUAGUGAACCAGUA
CCGUGAGGGAAAGGCGAAAAGAACCCCGGGAGGGGAGUGAAACAGAACCUGAAACCUGA
UGCAUACAAACAGUGGGAGCGCCCUAGUGGUGUGACUGCGUACCUUUUGUAUAAUGGGU
CAACGACUUACAUUCAGUAGCGAGCUUAACCGGAUAGGGGAGGCGUAGGGAAACCGAGU
CUUAAUAGGGCGAUGAGUUGCUGGGUGUAGACCCGAAACCGAGUGAUCUAUCCAUGGCC
AGGUUGAAGGUGCCGUAACAGGUACUGGAGGACCGAACCCACGCAUGUUGCAAAAUGCG
GGGAUGAGCUGUGGGUAGGGGUGAAAGGCUAAACAAACUCGGAGAUAGCUGGUUCUCCC
CGAAAACUAUUUAGGUAGUGCCUCGAGCAAGACACUGAUGGGGGUAAAGCACUGUUAUG
GCUAGGGGGUUAUUGCAACUUACCAACCCAUGGCAAACUCAGAAUACCAUCAAGUGGUUC
CUCGGGAGACAGACAGCGGGUGCUAACGUCCGUUGUCAAGAGGGAAACAACCCAGACCGC
CGGCUAAGGUCCCAAAUGAUAGAUUAAGUGGUAAACGAAGUGGGAAGGCACAGACAGCC
AGGAUGUUGGCUUAGAAGCAGCCAUCAUUUAAAGAAAGCGUAAUAGCUCACUGGUCGAG
UCGUCCUGCGCGGAAGAUGUAACGGGGCUCAAAUCUAUAACCGAAGCUGCGGAUGCCGGU
UUACCGGCAUGGUAGGGGAGCGUUCUGUAGGCUGAUGAAGGUGCAUUGUAAAGUGUGCU
GGAGGUAUCAGAAGUGCGAAUGUUGACAUGAGUAGCGAUAAAGCGGGUGAAAAGCCCGC
UCGCCGAAAGCCCAAGGUUUCCUACGCAACGUUCAUCGGCGUAGGGUGAGUCGGCCCCUA
AGGCGAGGCAGAAAUGCGUAGUCGAUGGGAAACAGGUUAAUAUUCCUGUACUUGAUUCA
AAUGCGAUGUGGGGACGGAGAAGGUUAGGUUGGCAAGCUGUUGGAAUAGCUUGUUUAAG
CCGGUAGGUGGAAGACUUAGGCAAAUCCGGGUUUUCUUAACACCGAGAAGUGAUGACGA
GUGUCUACGGACACGAAGCAACCGAUACCACGCUUCCAGGAAAAGCCACUAAGCUUCAGU
UUGAAUCGAACCGUACCGCAAACCGACACAGGUGGGCAGGAUGAGAAUUCUAAGGCGCU
UGAGAGAACUCGGGAGAAGGAACUCGGCAAAUUGAUACCGUAACUUCGGGAGAAGGUAU
GCCCUCUAAGGUUAAGGACUUGCUCCGUAAGCCCCGGAGGGUCGCAGAGAAUAGGUGGCU
GCGACUGUUUAUUAAAAACACAGCACUCUGCCAACACGAAAGUGGACGUAUAGGGUGUG
ACGCCUGCCCGGUGCCGGAAGGUUAAUUGAAGAUGUGCAAGCAUCGGAUCGAAGCCCCGG
UAAACGGCGGCCGUAACUAUAACGGUCCUAAGGUAGCGAAAUUCCUUGUCGGGUAAGUU
CCGACCCGCACGAAUGGCGUAACGAUGGCCACACUGUCUCCUCCCGAGACUCAGCGAAGU
UGAAGUGGUUGUGAAGAUGCAAUCUACCCGCUGCUAGACGGAAAGACCCCGUGAACCUU
UACUGUAGCUUUGCAUUGGACUUUGAAGUCACUUGUGUAGGAUAGGUGGGAGGCUUGGA
AGCAGAGACGCCAGUCUCUGUGGAGUCGUCCUUGAAAUACCACCCUGGUGUCUUUGAGGU
UCUAACCCAGACCCGUCAUCCGGGUCGGGGACCGUGCAUGGUAGGCAGUUUGACUGGGGC
GGUCUCCUCCCAAAGCGUAACGGAGGAGUUCGAAGGUUACCUAGGUCCGGUCGGAAAUCG
GACUGAUAGUGCAAUGGCAAAAGGUAGCUUAACUGCGAGACCGACAAGUCGGGCAGGUG
CGAAAGCAGGACAUAGUGAUCCGGUGGUUCUGUAUGGAAGGGCCAUCGCUCAACGGAUA
AAAGGUACUCCGGGGAUAACAGGCUGAUUCCGCCCAAGAGUUCAUAUCGACGGCGGAGU
UUGGCACCUCGAUGUCGGCUCAUCACAUCCUGGGGCUGUAGUCGGUCCCAAGGGUAUGGC
UGUUCGCCAUUUAAAGUGGUACGUGAGCUGGGUUUAAAACGUCGUGAGACAGUUUGGUC
CCUAUCUGCAGUGGGCGUUGGAAGUUUGACGGGGGCUGCUCCUAGUACGAGAGGACCGG
AGUGGACGAACCUCUGGUGUACCGGUUGUAACGCCAGUUGCAUAGCCGGGUAGCUAAGU
UCGGAAGAGAUAAGCGCUGAAAGCAUCUAAGCGCGAAACUCGCCUGAAGAUGAGACUUC
CCUUGCGGUUUAACCGCACUAAAGGGUCGUUCGAGACCAGGACGUUGAUAGGUGGGGUG
UGGAAGCGCGGUAACGCGUGAAGCUAACCCAUACUAAUUGCCCGUGAGGCUUGACUCU

> A9Y61_10505: 16S ribosomal RNA (1 of 4 copies) NZ_CP016017.1:1944811-1946362- Is on the negative strand DNA (- strand): SEQ ID NO: 25

TGAACATAAGAGTTTGATCCTGGCTCAGATTGAACGCTGGCGGCATGCTTTACACATGCAAG
TCGGACGGCAGCACAGGGAAGCTTGCTTCTCGGGTGGCGAGTGGCGAACGGGTGAGTAACA
TATCGGAACGTACCGGGTAGCGGGGGATAACTGATCGAAAGATCAGCTAATACCGCATACG
TCTTGAGAGGGAAAGCAGGGGACCTTCGGGCCTTGCGCTATCCGAGCGGCCGATATCTGATT
AGCTGGTTGGCGGGGTAAAGGCCCACCAAGGCGACGATCAGTAGCGGGTCTGAGAGGATGA
TCCGCCACACTGGGACTGAGACACGGCCCAGACTCCTACGGGAGGCAGCAGTGGGGAATTT
TGGACAATGGGCGCAAGCCTGATCCAGCCATGCCGCGTGTCTGAAGAAGGCCTTCGGGTTGT
AAAGGACTTTTGTCAGGGAAGAAAAGGCCGTTGCCAATATCGGCGGCCGATGACGGTACCT
GAAGAATAAGCACCGGCTAACTACGTGCCAGCAGCCGCGGTAATACGTAGGGTGCGAGCGT
TAATCGGAATTACTGGGCGTAAAGCGGGCGCAGACGGTTACTTAAGCAGGATGTGAAATCC
CCGGGCTCAACCCGGGAACTGCGTTCTGAACTGGGTGACTCGAGTGTGTCAGAGGGAGGTG
GAATTCCACGTGTAGCAGTGAAATGCGTAGAGATGTGGAGGAATACCGATGGCGAAGGCAG
CCTCCTGGGATAACACTGACGTTCATGTCCGAAAGCGTGGGTAGCAAACAGGATTAGATACC
CTGGTAGTCCACGCCCTAAACGATGTCAATTAGCTGTTGGGCAACTTGATTGCTTGGTAGCG
TAGCTAACGCGTGAAATTGACCGCCTGGGGAGTACGGTCGCAAGATTAAAACTCAAAGGAA
TTGACGGGGACCCGCACAAGCGGTGGATGATGTGGATTAATTCGATGCAACGCGAAGAACC
TTACCTGGTTTTGACATGTGCGGAATCCTCCGGAGACGGAGGAGTGCCTTCGGGAGCCGTAA
CACAGGTGCTGCATGGCTGTCGTCAGCTCGTGTCGTGAGATGTTGGGTTAAGTCCCGCAACG
AGCGCAACCCTTGTCATTAGTTGCCATCATTCGGTTGGGCACTCTAATGAGACTGCCGGTGA
CAAGCCGGAGGAAGGTGGGGATGACGTCAAGTCCTCATGGCCCTTATGACCAGGGCTTCAC
ACGTCATACAATGGTCGGTACAGAGGGTAGCCAAGCCGCGAGGCGGAGCCAATCTCACAAA
ACCGATCGTAGTCCGGATTGCACTCTGCAACTCGAGTGCATGAAGTCGGAATCGCTAGTAAT
CGCAGGTCAGCATACTGCGGTGAATACGTTCCCGGGTCTTGTACACACCGCCCGTCACACCA
TGGGAGTGGGGGATACCAGAAGTAGGTAGGGTAACCGCAAGGAGTCCGCTTACCACGGTAT
GCTTCATGACTGGGGTGAAGTCGTAACAAGGTAGCCGTAGGGGAACCTGCGGCTGGATCAC
CTCCTTTCTA

cDNA: SEQ ID NO: 26

TAGAAAGGAGGTGATCCAGCCGCAGGTTCCCCTACGGCTACCTTGTTACGACTTCACCCCAG
TCATGAAGCATACCGTGGTAAGCGGACTCCTTGCGGTTACCCTACCTACTTCTGGTATCCCCC
ACTCCCATGGTGTGACGGGCGGTGTGTACAAGACCCGGGAACGTATTCACCGCAGTATGCTG
ACCTGCGATTACTAGCGATTCCGACTTCATGCACTCGAGTTGCAGAGTGCAATCCGGACTAC
GATCGGTTTTGTGAGATTGGCTCCGCCTCGCGGCTTGGCTACCCTCTGTACCGACCATTGTAT
GACGTGTGAAGCCCTGGTCATAAGGGCCATGAGGACTTGACGTCATCCCCACCTTCCTCCGG
CTTGTCACCGGCAGTCTCATTAGAGTGCCCAACCGAATGATGGCAACTAATGACAAGGGTTG
CGCTCGTTGCGGGACTTAACCCAACATCTCACGACACGAGCTGACGACAGCCATGCAGCACC
TGTGTTACGGCTCCCGAAGGCACTCCTCCGTCTCCGGAGGATTCCGCACATGTCAAAACCAG
GTAAGGTTCTTCGCGTTGCATCGAATTAATCCACATCATCCACCGCTTGTGCGGGTCCCCGTC
AATTCCTTTGAGTTTTAATCTTGCGACCGTACTCCCCAGGCGGTCAATTTCACGCGTTAGCTA
CGCTACCAAGCAATCAAGTTGCCCAACAGCTAATTGACATCGTTTAGGGCGTGGACTACCAG
GGTATCTAATCCTGTTTGCTACCCACGCTTTCGGACATGAACGTCAGTGTTATCCCAGGAGG
CTGCCTTCGCCATCGGTATTCCTCCACATCTCTACGCATTTCACTGCTACACGTGGAATTCCA
CCTCCCTCTGACACACTCGAGTCACCCAGTTCAGAACGCAGTTCCCGGGTTGAGCCCGGGGA
TTTCACATCCTGCTTAAGTAACCGTCTGCGCCCGCTTTACGCCCAGTAATTCCGATTAACGCT
CGCACCCTACGTATTACCGCGGCTGCTGGCACGTAGTTAGCCGGTGCTTATTCTTCAGGTACC
GTCATCGGCCGCCGATATTGGCAACGGCCTTTTCTTCCCTGACAAAAGTCCTTTACAACCCG
AAGGCCTTCTTCAGACACGCGGCATGGCTGGATCAGGCTTGCGCCCATTGTCCAAAATTCCC
CACTGCTGCCTCCCGTAGGAGTCTGGGCCGTGTCTCAGTCCCAGTGTGGCGGATCATCCTCTC
AGACCCGCTACTGATCGTCGCCTTGGTGGGCCTTTACCCCGCCAACCAGCTAATCAGATATC
GGCCGCTCGGATAGCGCAAGGCCCGAAGGTCCCCTGCTTTCCCTCTCAAGACGTATGCGGTA
TTAGCTGATCTTTCGATCAGTTATCCCCCGCTACCCGGTACGTTCCGATATGTTACTCACCCG
TTCGCCACTCGCCACCCGAGAAGCAAGCTTCCCTGTGCTGCCGTCCGACTTGCATGTGTAAA
GCATGCCGCCAGCGTTCAATCTGAGCCAGGATCAAACTCTTATGTTCA

RNA: SEQ ID NO: 27

UGAACAUAAGAGUUUGAUCCUGGCUCAGAUUGAACGCUGGCGGCAUGCUUUACACAUGC
AAGUCGGACGGCAGCACAGGGAAGCUUGCUUCUCGGGUGGCGAGUGGCGAACGGGUGAG
UAACAUAUCGGAACGUACCGGGUAGCGGGGGAUAACUGAUCGAAAGAUCAGCUAAUACC
GCAUACGUCUUGAGAGGGAAAGCAGGGGACCUUCGGGCCUUGCGCUAUCCGAGCGGCCGA
UAUCUGAUUAGCUGGUUGGCGGGGUAAAGGCCCACCAAGGCGACGAUCAGUAGCGGGUC
UGAGAGGAUGAUCCGCCACACUGGGACUGAGACACGGCCCAGACUCCUACGGGAGGCAGC
AGUGGGGAAUUUUGGACAAUGGGCGCAAGCCUGAUCCAGCCAUGCCGCGUGUCUGAAGA
AGGCCUUCGGGUUGUAAAGGACUUUUGUCAGGGAAGAAAAGGCCGUUGCCAAUAUCGGC
GGCCGAUGACGGUACCUGAAGAAUAAGCACCGGCUAACUACGUGCCAGCAGCCGCGGUAA
UACGUAGGGUGCGAGCGUUAAUCGGAAUUACUGGGCGUAAAGCGGGCGCAGACGGUUAC
UUAAGCAGGAUGUGAAAUCCCCGGGCUCAACCCGGGAACUGCGUUCUGAACUGGGUGACU
CGAGUGUGUCAGAGGGAGGUGGAAUUCCACGUGUAGCAGUGAAAUGCGUAGAGAUGUGG
AGGAAUACCGAUGGCGAAGGCAGCCUCCUGGGAUAACACUGACGUUCAUGUCCGAAAGCG
UGGGUAGCAAACAGGAUUAGAUACCCUGGUAGUCCACGCCCUAAACGAUGUCAAUUAGC
UGUUGGGCAACUUGAUUGCUUGGUAGCGUAGCUAACGCGUGAAAUUGACCGCCUGGGGA
GUACGGUCGCAAGAUUAAAACUCAAAGGAAUUGACGGGGACCCGCACAAGCGGUGGAUG
AUGUGGAUUAAUUCGAUGCAACGCGAAGAACCUUACCUGGUUUUGACAUGUGCGGAAUC
CUCCGGAGACGGAGGAGUGCCUUCGGGAGCCGUAACACAGGUGCUGCAUGGCUGUCGUCA
GCUCGUGUCGUGAGAUGUUGGGUUAAGUCCCGCAACGAGCGCAACCCUUGUCAUUAGUU
GCCAUCAUUCGGUUGGGCACUCUAAUGAGACUGCCGGUGACAAGCCGGAGGAAGGUGGG
GAUGACGUCAAGUCCUCAUGGCCCUUAUGACCAGGGCUUCACACGUCAUACAAUGGUCGG
UACAGAGGGUAGCCAAGCCGCGAGGCGGAGCCAAUCUCACAAAACCGAUCGUAGUCCGGA
UUGCACUCUGCAACUCGAGUGCAUGAAGUCGGAAUCGCUAGUAAUCGCAGGUCAGCAUAC
UGCGGUGAAUACGUUCCCGGGUCUUGUACACACCGCCCGUCACACCAUGGGAGUGGGGGA
UACCAGAAGUAGGUAGGGUAACCGCAAGGAGUCCGCUUACCACGGUAUGCUUCAUGACU
GGGGUGAAGUCGUAACAAGGUAGCCGUAGGGGAACCUGCGGCUGGAUCACCUCCUUUCU
A

### ANNEX B

Sequences for the exemplary marker genes differentially expressed between an untreated sample and a sample treated with antibiotics

### 1. porB (Locus Tag: NGO1812)

### Ngo1812: Nc_002946.2:1788697-1789744

DNA (+ strand): SEQ ID NO: 28

ATGAAAAAATCCCTGATTGCCCTGACTTTGGCAGCCCTTCCTGTTGCGGCAATGGCC
GATGTCACCCTGTACGGCGCCATCAAAGCCGGCGTACAAACTTACCGTTCTGTAGAA
CATACAGACGGCAAGGTAAGTAAAGTGGAAACCGGCAGCGAAATCGCCGACTTCGG
TTCAAAAATCGGCTTCAAAGGCCAAGAAGACCTCGGCAACGGCCTGAAGGCCGTTT
GGCAGTTGGAACAAGGTGCCTCCGTCGCCGGCACTAACACCGGCTGGGGCAACAAA
CAATCCTTCGTCGGCTTGAAGGGCGGCTTCGGTACCATCCGCGCCGGTAGCCTGAAC
AGCCCCCTGAAAAACACCGGCGCCAACGTCAATGCTTGGGAATCCGGCAAATTTAC
CGGCAATGTGCTGGAAATCAGCGGAATGGCCCAACGGGAACACCGCTACCTGTCCG
TACGCTACGATTCTCCCGAATTTGCCGGCTTCAGCGGCAGCGTACAATACGCACCTA
AAGACAATTCAGGCTCAAACGGCGAATCTTACCACGTTGGCTTGAACTACCAAAAC
AGCGGCTTCTTCGCGCAATACGCCGGCTTGTTCCAAAGATACGGCGAAGGCACTAA
AAAAATCGAATACGATGGTCAAACTTATAGTATCCCCAGTCTGTTTGTTGAAAAACT
GCAAGTTCACCGTTTGGTAGGCGGTTACGACAATAATGCCCTGTACGTTTCCGTAGC
CGCACAACAACAAGATGCCAAATTGTATGGAGCAATGAGCGGTAATTCGCACAACT
CTCAAACCGAAGTTGCCGCTACCGCGGCATACCGTTTCGGCAATGTAACGCCCCGCG
TTTCTTACGCCCACGGCTTCAAAGGCACTGTTGATAGTGCAAACCACGACAATACTT
ATGACCAAGTGGTTGTCGGTGCGGAATACGACTTCTCCAAACGCACTTCTGCCTTGG
TTTCTGCCGGCTGGTTGCAAGAAGGCAAAGGCGCAGACAAAATCGTATCGACTGCC
AGCGCCGTCGTTCTGCGCCACAAATTCTAA

cDNA: SEQ ID NO: 29

TTAGAATTTGTGGCGCAGAACGACGGCGCTGGCAGTCGATACGATTTTGTCTGCGCC
TTTGCCTTCTTGCAACCAGCCGGCAGAAACCAAGGCAGAAGTGCGTTTGGAGAAGT
CGTATTCCGCACCGACAACCACTTGGTCATAAGTATTGTCGTGGTTTGCACTATCAA
CAGTGCCTTTGAAGCCGTGGGCGTAAGAAACGCGGGGCGTTACATTGCCGAAACGG
TATGCCGCGGTAGCGGCAACTTCGGTTTGAGAGTTGTGCGAATTACCGCTCATTGCT
CCATACAATTTGGCATCTTGTTGTTGTGCGGCTACGGAAACGTACAGGGCATTATTG
TCGTAACCGCCTACCAAACGGTGAACTTGCAGTTTTTCAACAAACAGACTGGGGATA
CTATAAGTTTGACCATCGTATTCGATTTTTTTAGTGCCTTCGCCGTATCTTTGGAACA
AGCCGGCGTATTGCGCGAAGAAGCCGCTGTTTTGGTAGTTCAAGCCAACGTGGTAA
GATTCGCCGTTTGAGCCTGAATTGTCTTTAGGTGCGTATTGTACGCTGCCGCTGAAG
CCGGCAAATTCGGGAGAATCGTAGCGTACGGACAGGTAGCGGTGTTCCCGTTGGGC
CATTCCGCTGATTTCCAGCACATTGCCGGTAAATTTGCCGGATTCCCAAGCATTGAC
GTTGGCGCCGGTGTTTTTCAGGGGGCTGTTCAGGCTACCGGCGCGGATGGTACCGAA
GCCGCCCTTCAAGCCGACGAAGGATTGTTTGTTGCCCCAGCCGGTGTTAGTGCCGGC
GACGGAGGCACCTTGTTCCAACTGCCAAACGGCCTTCAGGCCGTTGCCGAGGTCTTC
TTGGCCTTTGAAGCCGATTTTTGAACCGAAGTCGGCGATTTCGCTGCCGGTTTCCACT
TTACTTACCTTGCCGTCTGTATGTTCTACAGAACGGTAAGTTTGTACGCCGGCTTTGA
TGGCGCCGTACAGGGTGACATCGGCCATTGCCGCAACAGGAAGGGCTGCCAAAGTC
AGGGCAATCAGGGATTTTTTCAT

RNA: SEQ ID NO: 30

AUGAAAAAAUCCCUGAUUGCCCUGACUUUGGCAGCCCUUCCUGUUGCGGCAAUGG
CCGAUGUCACCCUGUACGGCGCCAUCAAAGCCGGCGUACAAACUUACCGUUCUGU
AGAACAUACAGACGGCAAGGUAAGUAAAGUGGAAACCGGCAGCGAAAUCGCCGA
CUUCGGUUCAAAAAUCGGCUUCAAAGGCCAAGAAGACCUCGGCAACGGCCUGAAG
GCCGUUUGGCAGUUGGAACAAGGUGCCUCCGUCGCCGGCACUAACACCGGCUGGG
GCAACAAACAAUCCUUCGUCGGCUUGAAGGGCGGCUUCGGUACCAUCCGCGCCGG
UAGCCUGAACAGCCCCCUGAAAAACACCGGCGCCAACGUCAAUGCUUGGGAAUCC
GGCAAAUUUACCGGCAAUGUGCUGGAAAUCAGCGGAAUGGCCCAACGGGAACACC
GCUACCUGUCCGUACGCUACGAUUCUCCCGAAUUUGCCGGCUUCAGCGGCAGCGU
ACAAUACGCACCUAAAGACAAUUCAGGCUCAAACGGCGAAUCUUACCACGUUGGC
UUGAACUACCAAAACAGCGGCUUCUUCGCGCAAUACGCCGGCUUGUUCCAAAGAU
ACGGCGAAGGCACUAAAAAAAUCGAAUACGAUGGUCAAACUUAUAGUAUCCCCA
GUCUGUUUGUUGAAAAACUGCAAGUUCACCGUUUGGUAGGCGGUUACGACAAUA
AUGCCCUGUACGUUUCCGUAGCCGCACAACAACAAGAUGCCAAAUUGUAUGGAGC
AAUGAGCGGUAAUUCGCACAACUCUCAAACCGAAGUUGCCGCUACCGCGGCAUAC
CGUUUCGGCAAUGUAACGCCCCGCGUUUCUUACGCCCACGGCUUCAAAGGCACUG
UUGAUAGUGCAAACCACGACAAUACUUAUGACCAAGUGGUUGUCGGUGCGGAAU
ACGACUUCUCCAAACGCACUUCUGCCUUGGUUUCUGCCGGCUGGUUGCAAGAAGG
CAAAGGCGCAGACAAAAUCGUAUCGACUGCCAGCGCCGUCGUUCUGCGCCACAAA
UUCUAA

### 2. rpmB (Locus Tag: NGO1680

### NGO1680: NC_002946.2: C1633854-1633621

DNA (- strand): SEQ ID NO: 31

ATGGCACGAGTTTGCAAAGTGACCGGTAAACGCCCGATGTCCGGCAACAACGTATC
GCACGCCAACAACAAAACCAAACGCCGTTTTTTGCCCAACTTGCAATCACGTCGTTT
TTGGGTAGAAAGTGAAAACCGCTGGGTTCGCCTGCGCGTTTCCAACGCTGCATTGCG
TACCATCGACAAAGTAGGCATTGATGTCGTATTGGCTGATTTGCGTGCTCGCGGCGA
AGCTTAA

cDNA: SEQ ID NO: 32

TTAAGCTTCGCCGCGAGCACGCAAATCAGCCAATACGACATCAATGCCTACTTTGTC
GATGGTACGCAATGCAGCGTTGGAAACGCGCAGGCGAACCCAGCGGTTTTCACTTTC
TACCCAAAAACGACGTGATTGCAAGTTGGGCAAAAAACGGCGTTTGGTTTTGTTGTT
GGCGTGCGATACGTTGTTGCCGGACATCGGGCGTTTACCGGTCACTTTGCAAACTCG
TGCCAT

RNA: SEQ ID NO: 33

AUGGCACGAGUUUGCAAAGUGACCGGUAAACGCCCGAUGUCCGGCAACAACGUAU
CGCACGCCAACAACAAAACCAAACGCCGUUUUUUGCCCAACUUGCAAUCACGUCG
UUUUUGGGUAGAAAGUGAAAACCGCUGGGUUCGCCUGCGCGUUUCCAACGCUGC
AUUGCGUACCAUCGACAAAGUAGGCAUUGAUGUCGUAUUGGCUGAUUUGCGUGC
UCGCGGCGAAGCUUAA

### 3. NGO0016: NC_002946.2:c14431-14081

DNA (- strand): SEQ ID NO: 34

ATGGAAGCCTTCAAAACCCTAATTTGGATTATTAATATTATTTCCGCTTTGGCCGTCA
TCGTGTTAGTATTGCTCCAACACGGCAAAGGCGCGGATGCCGGCGCGACCTTCGGAT
CGGGAAGCGGCAGCGCGCAAGGCGTATTCGGCTCTGCCGGCAACGCCAACTTCCTC
AGCCGCTCGACCGCCGTTGCAGCAACATTTTTCTTTGCAACCTGCATGGCTATGGTG
TATATTCACACCCACACGACAAAACACGGTTTGGACTTCAGCAACATACGACAGAC
TCAGCAAGCACCCAAACCCGTAAGCAATACCGAACCTTCTGCCCCTGTTCCTCAGCA
GCAGAAATAA

cDNA: SEQ ID NO: 35

TTATTTCTGCTGCTGAGGAACAGGGGCAGAAGGTTCGGTATTGCTTACGGGTTTGGG
TGCTTGCTGAGTCTGTCGTATGTTGCTGAAGTCCAAACCGTGTTTTGTCGTGTGGGTG
TGAATATACACCATAGCCATGCAGGTTGCAAAGAAAAATGTTGCTGCAACGGCGGT
CGAGCGGCTGAGGAAGTTGGCGTTGCCGGCAGAGCCGAATACGCCTTGCGCGCTGC
CGCTTCCCGATCCGAAGGTCGCGCCGGCATCCGCGCCTTTGCCGTGTTGGAGCAATA
CTAACACGATGACGGCCAAAGCGGAAATAATATTAATAATCCAAATTAGGGTTTTG
AAGGCTTCCAT

RNA: SEQ ID NO: 36

AUGGAAGCCUUCAAAACCCUAAUUUGGAUUAUUAAUAUUAUUUCCGCUUUGGCC
GUCAUCGUGUUAGUAUUGCUCCAACACGGCAAAGGCGCGGAUGCCGGCGCGACCU
UCGGAUCGGGAAGCGGCAGCGCGCAAGGCGUAUUCGGCUCUGCCGGCAACGCCAA
CUUCCUCAGCCGCUCGACCGCCGUUGCAGCAACAUUUUUCUUUGCAACCUGCAUG
GCUAUGGUGUAUAUUCACACCCACACGACAAAACACGGUUUGGACUUCAGCAACA
UACGACAGACUCAGCAAGCACCCAAACCCGUAAGCAAUACCGAACCUUCUGCCCC
UGUUCCUCAGCAGCAGAAAUAA

### 4. NGO0171: NC_002946.2:c174519-174154

DNA (- strand): SEQ ID NO: 37

ATGAACCTGATTCAACAGCTCGAGCAAGAAGAAATTGCCCGCCTGAACAAAGAAAT
CCCCGAATTCGCACCGGGCGACACCGTAGTCGTATCCGTACGCGTCGTGGAAGGTA
CCCGCAGCCGTCTGCAAGCCTACGAAGGCGTGGTTATCGCCCGTCGCAACCGTGGTT
TGAACAGCAACTTCATCGTCCGCAAAATCTCCAGCGGCGAAGGTGTTGAACGTACTT
TCCAACTGTATTCCCCTACTGTTGAGAAAATCGAAGTCAAACGCCGTGGCGACGTAC
GCCGTGCCAAACTGTACTACCTGCGCGGTCTGACCGGCAAAGCTGCACGCATCAAA
GAAAAACTGCCTGCACGCAAAGGTTGA

cDNA: SEQ ID NO: 38

TCAACCTTTGCGTGCAGGCAGTTTTTCTTTGATGCGTGCAGCTTTGCCGGTCAGACCG
CGCAGGTAGTACAGTTTGGCACGGCGTACGTCGCCACGGCGTTTGACTTCGATTTTC
TCAACAGTAGGGGAATACAGTTGGAAAGTACGTTCAACACCTTCGCCGCTGGAGAT
TTTGCGGACGATGAAGTTGCTGTTCAAACCACGGTTGCGACGGGCGATAACCACGCC
TTCGTAGGCTTGCAGACGGCTGCGGGTACCTTCCACGACGCGTACGGATACGACTAC
GGTGTCGCCCGGTGCGAATTCGGGGATTTCTTTGTTCAGGCGGGCAATTTCTTCTTGC
TCGAGCTGTTGAATCAGGTTCAT

RNA: SEQ ID NO: 39

AUGAACCUGAUUCAACAGCUCGAGCAAGAAGAAAUUGCCCGCCUGAACAAAGAA
AUCCCCGAAUUCGCACCGGGCGACACCGUAGUCGUAUCCGUACGCGUCGUGGAAG
GUACCCGCAGCCGUCUGCAAGCCUACGAAGGCGUGGUUAUCGCCCGUCGCAACCG
UGGUUUGAACAGCAACUUCAUCGUCCGCAAAAUCUCCAGCGGCGAAGGUGUUGA
ACGUACUUUCCAACUGUAUUCCCCUACUGUUGAGAAAAUCGAAGUCAAACGCCGU
GGCGACGUACGCCGUGCCAAACUGUACUACCUGCGCGGUCUGACCGGCAAAGCUG
CACGCAUCAAAGAAAAACUGCCUGCACGCAAAGGUUGA

### 5. NGO0172: NC_002946.2:c175283-174534

DNA (- strand): SEQ ID NO: 40

ATGCTTATCCAGGCAGTTACCATTTTCCCCGAAATGTTCGACAGCATTACCCGCTAC
GGCGTAACGGGACGCGCGAACAGACAGGGAATCTGGCAGTTTGAAGCAGTCAATCC
CCGAAAGTTTGCCGACAACAGATTGGGCTATATCGACGACCGCCCGTTCGGCGGCG
GTCCGGGAATGATTATGATGGCTCCGCCGCTTCATGCGGCGATAGAACACGCCAAA
GCACAATCTTCCCAAACCGCAAAAGTCATCTACCTCAGCCCCCAAGGAAAACCGCT
GACACACCAAAAAGCGGCAGAACTGGCAGAACTTACGCATCTGATTCTGCTGTGCG
GACGCTATGAGGGAATAGACGAAAGACTGCTGCAAAGCAGCGTCGATGAAGAAATC
AGCATCGGAGACTTCGTCGTTTCCGGCGGAGAGCTTCCCGCCATGATGCTGATGGAT
GCGGTATTGAGGCTCGTACCCGGCATATTGGGCGACATTCAGTCTGCCGAACAGGAT
TCGTTCTCAAGCGGTATTTTGGACTGCCCCCACTACACCAAACCCTTAGAATTTCAA
GGCATGGCTGTTCCGGAAGTATTGCGCTCCGGAAATCATGGCTTGATAGCGGAATGG
CGGTTGGAACAATCGCTGCGCCGCACCTTGGAGCGCAGACCCGATCTTTTGGAAAA
GCGCGTTTTAATCCCAAAGGAATCCCGCCTCTTGAATAAAATCCTACAAGAGCAACG
GGAAATCCAATCATAA

cDNA: SEQ ID NO: 41

TTATGATTGGATTTCCCGTTGCTCTTGTAGGATTTTATTCAAGAGGCGGGATTCCTTT
GGGATTAAAACGCGCTTTTCCAAAAGATCGGGTCTGCGCTCCAAGGTGCGGCGCAG
CGATTGTTCCAACCGCCATTCCGCTATCAAGCCATGATTTCCGGAGCGCAATACTTC
CGGAACAGCCATGCCTTGAAATTCTAAGGGTTTGGTGTAGTGGGGGCAGTCCAAAA
TACCGCTTGAGAACGAATCCTGTTCGGCAGACTGAATGTCGCCCAATATGCCGGGTA
CGAGCCTCAATACCGCATCCATCAGCATCATGGCGGGAAGCTCTCCGCCGGAAACG
ACGAAGTCTCCGATGCTGATTTCTTCATCGACGCTGCTTTGCAGCAGTCTTTCGTCTA
TTCCCTCATAGCGTCCGCACAGCAGAATCAGATGCGTAAGTTCTGCCAGTTCTGCCG
CTTTTTGGTGTGTCAGCGGTTTTCCTTGGGGGCTGAGGTAGATGACTTTTGCGGTTTG
GGAAGATTGTGCTTTGGCGTGTTCTATCGCCGCATGAAGCGGCGGAGCCATCATAAT
CATTCCCGGACCGCCGCCGAACGGGCGGTCGTCGATATAGCCCAATCTGTTGTCGGC
AAACTTTCGGGGATTGACTGCTTCAAACTGCCAGATTCCCTGTCTGTTCGCGCGTCCC
GTTACGCCGTAGCGGGTAATGCTGTCGAACATTTCGGGGAAAATGGTAACTGCCTGG
ATAAGCAT

RNA: SEQ ID NO: 42

AUGCUUAUCCAGGCAGUUACCAUUUUCCCCGAAAUGUUCGACAGCAUUACCCGCU
ACGGCGUAACGGGACGCGCGAACAGACAGGGAAUCUGGCAGUUUGAAGCAGUCA
AUCCCCGAAAGUUUGCCGACAACAGAUUGGGCUAUAUCGACGACCGCCCGUUCGG
CGGCGGUCCGGGAAUGAUUAUGAUGGCUCCGCCGCUUCAUGCGGCGAUAGAACAC
GCCAAAGCACAAUCUUCCCAAACCGCAAAAGUCAUCUACCUCAGCCCCCAAGGAA
AACCGCUGACACACCAAAAAGCGGCAGAACUGGCAGAACUUACGCAUCUGAUUCU
GCUGUGCGGACGCUAUGAGGGAAUAGACGAAAGACUGCUGCAAAGCAGCGUCGA
UGAAGAAAUCAGCAUCGGAGACUUCGUCGUUUCCGGCGGAGAGCUUCCCGCCAUG
AUGCUGAUGGAUGCGGUAUUGAGGCUCGUACCCGGCAUAUUGGGCGACAUUCAG
UCUGCCGAACAGGAUUCGUUCUCAAGCGGUAUUUUGGACUGCCCCCACUACACCA
AACCCUUAGAAUUUCAAGGCAUGGCUGUUCCGGAAGUAUUGCGCUCCGGAAAUC
AUGGCUUGAUAGCGGAAUGGCGGUUGGAACAAUCGCUGCGCCGCACCUUGGAGC
GCAGACCCGAUCUUUUGGAAAAGCGCGUUUUAAUCCCAAAGGAAUCCCGCCUCUU
GAAUAAAAUCCUACAAGAGCAACGGGAAAUCCAAUCAUAA

### 6. NGO0173: NC_002946.2:c175792-175283

DNA (- strand): SEQ ID NO: 43

ATGACAGACACTCAAAACCGGGTAGCCATGGGCTACATCAAAGGCGTATTCGGCAT
AAAAGGCTGGCTGAAAATTGCCGCCAACACCGAATATTCCGACAGCCTTTTGGACTA
CCCCGAGTGGCATTTGGCCAAGGACGGCAAAACCGTCAGCGTTACCCTTGAAGCCG
GAAAAGTCGTCAACGGCGAACTCCAAGTCAAATTCGAAGGCATAGACGACCGCGAT
TCAGCATTCTCATTGCGCGGTTACACCATCGAAATACCCCGTGAAGCATTCGCCCCG
ACAGAAGAAGACGAATACTACTGGGCAGACTTGGTCGGCATGACCGTCGTCAACAA
AGACGATACCGTTTTAGGCAAGGTAAGCAACCTGATGGAAACCGGCGCAAACGACG
TATTGATGATTGACGGAGAACACGGGCAGATTCTGATTCCGTTCGTTTCCCAATATA
TCGAAACCGTCGATACCGGCAGCAAGACCATTACTGCCGACTGGGGTTTGGACTACT
GA

cDNA: SEQ ID NO: 44

TCAGTAGTCCAAACCCCAGTCGGCAGTAATGGTCTTGCTGCCGGTATCGACGGTTTC
GATATATTGGGAAACGAACGGAATCAGAATCTGCCCGTGTTCTCCGTCAATCATCAA
TACGTCGTTTGCGCCGGTTTCCATCAGGTTGCTTACCTTGCCTAAAACGGTATCGTCT
TTGTTGACGACGGTCATGCCGACCAAGTCTGCCCAGTAGTATTCGTCTTCTTCTGTCG
GGGCGAATGCTTCACGGGGTATTTCGATGGTGTAACCGCGCAATGAGAATGCTGAA
TCGCGGTCGTCTATGCCTTCGAATTTGACTTGGAGTTCGCCGTTGACGACTTTTCCGG
CTTCAAGGGTAACGCTGACGGTTTTGCCGTCCTTGGCCAAATGCCACTCGGGGTAGT
CCAAAAGGCTGTCGGAATATTCGGTGTTGGCGGCAATTTTCAGCCAGCCTTTTATGC
CGAATACGCCTTTGATGTAGCCCATGGCTACCCGGTTTTGAGTGTCTGTCAT

RNA: SEQ ID NO: 45

AUGACAGACACUCAAAACCGGGUAGCCAUGGGCUACAUCAAAGGCGUAUUCGGCA
UAAAAGGCUGGCUGAAAAUUGCCGCCAACACCGAAUAUUCCGACAGCCUUUUGGA
CUACCCCGAGUGGCAUUUGGCCAAGGACGGCAAAACCGUCAGCGUUACCCUUGAA
GCCGGAAAAGUCGUCAACGGCGAACUCCAAGUCAAAUUCGAAGGCAUAGACGACC
GCGAUUCAGCAUUCUCAUUGCGCGGUUACACCAUCGAAAUACCCCGUGAAGCAUU
CGCCCCGACAGAAGAAGACGAAUACUACUGGGCAGACUUGGUCGGCAUGACCGUC
GUCAACAAAGACGAUACCGUUUUAGGCAAGGUAAGCAACCUGAUGGAAACCGGC
GCAAACGACGUAUUGAUGAUUGACGGAGAACACGGGCAGAUUCUGAUUCCGUUC
GUUUCCCAAUAUAUCGAAACCGUCGAUACCGGCAGCAAGACCAUUACUGCCGACU
GGGGUUUGGACUACUGA

### 7. NGO0174: NC_002946.2:c176053-175808

DNA (- strand): SEQ ID NO: 46

ATGGTAGTTATCCGTTTGGCACGCGGCGGCTCGAAACACCGCCCCTTCTACAACGTC
ATCGTTACTGACTCACGCAGCCGCCGCGACGGCCGCTTCATCGAACGCGTAGGCTTC
TACAACCCCGTAGCCAACGAAAAACAAGAGCGCGTCCGCCTCAATGCAGACCGCCT
GAACCACTGGATTGCACAAGGCGCGCAAGTCAGCGACTCCGTTGCAAAACTGATTA
AAGAACAAAAAGCCGTCTAA

cDNA: SEQ ID NO: 47

TTAGACGGCTTTTTGTTCTTTAATCAGTTTTGCAACGGAGTCGCTGACTTGCGCGCCT
TGTGCAATCCAGTGGTTCAGGCGGTCTGCATTGAGGCGGACGCGCTCTTGTTTTTCG
TTGGCTACGGGGTTGTAGAAGCCTACGCGTTCGATGAAGCGGCCGTCGCGGCGGCT
GCGTGAGTCAGTAACGATGACGTTGTAGAAGGGGCGGTGTTTCGAGCCGCCGCGTG
CCAAACGGATAACTACCAT

RNA: SEQ ID NO: 48

AUGGUAGUUAUCCGUUUGGCACGCGGCGGCUCGAAACACCGCCCCUUCUACAACG
UCAUCGUUACUGACUCACGCAGCCGCCGCGACGGCCGCUUCAUCGAACGCGUAGG
CUUCUACAACCCCGUAGCCAACGAAAAACAAGAGCGCGUCCGCCUCAAUGCAGAC
CGCCUGAACCACUGGAUUGCACAAGGCGCGCAAGUCAGCGACUCCGUUGCAAAAC
UGAUUAAAGAACAAAAAGCCGUCUAA

### 8. Ngo0340: Nc_002946.2:334760-335692

DNA (+ strand): SEQ ID NO: 49

ATGAAAATTGCAAACAGCATCACCGAATTGATCGGCAACACGCCTTTGGTCAAACT
GAACCGTTTGACCAAAGGTTTGAAGGCAGAGGTTGCCGTGAAACTGGAATTTTTTAA
TCCGGGCAGCAGCGTCAAAGACCGCATTGCCGAAGCAATGATCGAGGCCGCCGAAA
AAGCGGGAAAAATCAACAAAAACACCGTCATTGTCGAAGCAACCAGCGGCAATAC
GGGTATCGGTTTGGCAATGGTATGTGCCGCACGCGGCTACAAACTGGCGATTACCAT
GCCGGAAAGCATGAGCAAAGAGCGCAAAATGCTGTTGCGCACGTTTGGCGCGGAAC
TGATTCTAACCCCCGCCGCCGAAGGTATGGCGGGCGCGATTGCCAAAGCGCAATCC
TTGGTGGACGCTCATCCAGACACTTATTTTATGCCGCGCCAGTTCGACAATGAGGCA
AATCCCGAAGTCCACCGCAAAACAACCGCCGAGGAAATTTGGAACGATACCGACGG
TAAAGTCGATGTCTTCGTTGCCGGCGTCGGCACGGGCGGTACGATTACCGGCGTGGG
CGAAGTGTTGAAAAAATACAAACCCGAAATTGAAGTGTGTGCCGTCGAAGCTGGCG
CTTCCCCCGTATTGAGCGGCGGCGAAAAAGGTCCGCACCCGATTCAAGGTATCGGC
GCAGGTTTTATTCCGACCGTTTTGAATACCAAAATCTACGACAGCATTGCCAAAGTG
CCGAACGAAGCGGCTTTTGAAACCGCCCGTGCAATGGCGGAAAAAGAAGGCATTTT
GGCGGGCATTTCTTCCGGTGCGGCGGTTTGGAGCGCGTTGCAGCTTGCCAAACAGCC
TGAAAACGAAGGCAAGCTGATAGTCGTGCTGCTGCCTTCTTATGGCGAACGCTATCT
TTCTACGCCACTTTTTGCAGATTTGGCATAA

cDNA: SEQ ID NO: 50

TTATGCCAAATCTGCAAAAAGTGGCGTAGAAAGATAGCGTTCGCCATAAGAAGGCA
GCAGCACGACTATCAGCTTGCCTTCGTTTTCAGGCTGTTTGGCAAGCTGCAACGCGC
TCCAAACCGCCGCACCGGAAGAAATGCCCGCCAAAATGCCTTCTTTTTCCGCCATTG
CACGGGCGGTTTCAAAAGCCGCTTCGTTCGGCACTTTGGCAATGCTGTCGTAGATTT
TGGTATTCAAAACGGTCGGAATAAAACCTGCGCCGATACCTTGAATCGGGTGCGGA
CCTTTTTCGCCGCCGCTCAATACGGGGGAAGCGCCAGCTTCGACGGCACACACTTCA
ATTTCGGGTTTGTATTTTTTCAACACTTCGCCCACGCCGGTAATCGTACCGCCCGTGC
CGACGCCGGCAACGAAGACATCGACTTTACCGTCGGTATCGTTCCAAATTTCCTCGG
CGGTTGTTTTGCGGTGGACTTCGGGATTTGCCTCATTGTCGAACTGGCGCGGCATAA
AATAAGTGTCTGGATGAGCGTCCACCAAGGATTGCGCTTTGGCAATCGCGCCCGCCA
TACCTTCGGCGGCGGGGGTTAGAATCAGTTCCGCGCCAAACGTGCGCAACAGCATTT
TGCGCTCTTTGCTCATGCTTTCCGGCATGGTAATCGCCAGTTTGTAGCCGCGTGCGGC
ACATACCATTGCCAAACCGATACCCGTATTGCCGCTGGTTGCTTCGACAATGACGGT
GTTTTTGTTGATTTTTCCCGCTTTTTCGGCGGCCTCGATCATTGCTTCGGCAATGCGG
TCTTTGACGCTGCTGCCCGGATTAAAAAATTCCAGTTTCACGGCAACCTCTGCCTTCA
AACCTTTGGTCAAACGGTTCAGTTTGACCAAAGGCGTGTTGCCGATCAATTCGGTGA
TGCTGTTTGCAATTTTCAT

RNA: SEQ ID NO 51

AUGAAAAUUGCAAACAGCAUCACCGAAUUGAUCGGCAACACGCCUUUGGUCAAAC
UGAACCGUUUGACCAAAGGUUUGAAGGCAGAGGUUGCCGUGAAACUGGAAUUUU
UUAAUCCGGGCAGCAGCGUCAAAGACCGCAUUGCCGAAGCAAUGAUCGAGGCCGC
CGAAAAAGCGGGAAAAAUCAACAAAAACACCGUCAUUGUCGAAGCAACCAGCGGC
AAUACGGGUAUCGGUUUGGCAAUGGUAUGUGCCGCACGCGGCUACAAACUGGCG
AUUACCAUGCCGGAAAGCAUGAGCAAAGAGCGCAAAAUGCUGUUGCGCACGUUU
GGCGCGGAACUGAUUCUAACCCCCGCCGCCGAAGGUAUGGCGGGCGCGAUUGCCA
AAGCGCAAUCCUUGGUGGACGCUCAUCCAGACACUUAUUUUAUGCCGCGCCAGUU
CGACAAUGAGGCAAAUCCCGAAGUCCACCGCAAAACAACCGCCGAGGAAAUUUGG
AACGAUACCGACGGUAAAGUCGAUGUCUUCGUUGCCGGCGUCGGCACGGGCGGUA
CGAUUACCGGCGUGGGCGAAGUGUUGAAAAAAUACAAACCCGAAAUUGAAGUGU
GUGCCGUCGAAGCUGGCGCUUCCCCCGUAUUGAGCGGCGGCGAAAAAGGUCCGCA
CCCGAUUCAAGGUAUCGGCGCAGGUUUUAUUCCGACCGUUUUGAAUACCAAAAUC
UACGACAGCAUUGCCAAAGUGCCGAACGAAGCGGCUUUUGAAACCGCCCGUGCAA
UGGCGGAAAAAGAAGGCAUUUUGGCGGGCAUUUCUUCCGGUGCGGCGGUUUGGA
GCGCGUUGCAGCUUGCCAAACAGCCUGAAAACGAAGGCAAGCUGAUAGUCGUGCU
GCUGCCUUCUUAUGGCGAACGCUAUCUUUCUACGCCACUUUUUGCAGAUUUGGCA
UAA

### 9. Ngo0592: Nc_002946.2:578772-580085

DNA (+ strand): SEQ ID NO: 52

ATGATGAGCGTAACTGTTGAAATTTTAGAAAATCTGGAACGCAAAGTAGTGTTGTCC
CTGCCTTGGTCCGAAATCAACGCAGAAACCGATAAAAAACTGAAACAAACCCAACG
CCGTGCAAAAATCGACGGTTTCCGTCCGGGTAAAGCACCTTTAAAAATGATTGCCCA
AATGTACGGTGCGAGCGCGCAAAACGACGTGATCAACGAGCTGGTGCAACGCCGCT
TCTACGATGTTGCCGTTGCCCAAGAGTTGAAAGTGGCAGGCTATCCACGTTTTGAAG
GCGTTGAAGAACAAGACGATAAAGAGTCTTTCAAAGTTGCCGCCATTTTTGAAGTGT
TCCCCGAAGTCGTTATCGGCGATTTGTCTGCACAAGAGGTCGAAAAAGTAACCGCTT
CCGTCGGCGATGCCGAAGTCGACCAAACTGTAGAAATCCTGCGTAAACAACGTACC
CGCTTCAACCATGTTGACCGCGAAGCCCGAAACGGCGACCGCGTCATCATCGACTTT
GAAGGCAAAATCGACGGCGAACCTTTTGCCGGCGGCACATCCAAAAACTACGCCTT
CGTATTGGGCGCAGGTCAAATGCTGCCTGAATTTGAAGCCGGCGTAGTCGGCATGA
AAGCGGGCGAAAGTAAAGACGTTACCGTCAACTTCCCTGAAGAATACCACGGCAAA
GATGTTGCCGGTAAAACTGCCGTGTTCACCATTACGCTGAACAATGTTTCCGAGCCC
ACTCTGCCTGAAGTCGATGCAGATTTTGCAAAAGCCTTGGGTATTGCGGATGGCGAT
GTTGCCAAAATGCGTGAAGAAGTGAAGAAAAACGTAAGCCGCGAAGTGGAACGCC
GCGTGAACGAACAAACCAAAGAATCCGTAATGAACGCGCTGATTAAAGCCGTAGAG
TTGAAAGTTCCGGTTGCTTTGGTCAATGAAGAAGCCGCCCGCCTGGCAAACGAAATG
AAACAAAACTTCGTTAACCAAGGTATGACCGATGCCGCGAACTTGGATTTGCCTTTG
GATATGTTCAAAGAACAAGCCGAACGTCGCGTATCTTTGGGTCTGATTTTGGCCAAA
CTGGTTGACGAAAACAAACTGGAACCGACTGAAGGGCAAATCAAAGCCGTTGTCGC
CAACTTCGCAGAAAGCTACGAAGATCCTCAAGAAGTGATTGACTGGTACTACGCAG
ATACTTCCCGCCTGCAAGCCCCGACTTCTTTGGCAGTAGAAAGCAACGTTGTTGATT
TCGTTTTGGGCAAAGCCAAAGTAAACAAAAAAGCTTTGTCTTTTGACGAAGTGATGG
GCGCGCAAGCCTGA

cDNA: SEQ ID NO: 53

TCAGGCTTGCGCGCCCATCACTTCGTCAAAAGACAAAGCTTTTTTGTTTACTTTGGCT
TTGCCCAAAACGAAATCAACAACGTTGCTTTCTACTGCCAAAGAAGTCGGGGCTTGC
AGGCGGGAAGTATCTGCGTAGTACCAGTCAATCACTTCTTGAGGATCTTCGTAGCTT
TCTGCGAAGTTGGCGACAACGGCTTTGATTTGCCCTTCAGTCGGTTCCAGTTTGTTTT
CGTCAACCAGTTTGGCCAAAATCAGACCCAAAGATACGCGACGTTCGGCTTGTTCTT
TGAACATATCCAAAGGCAAATCCAAGTTCGCGGCATCGGTCATACCTTGGTTAACGA
AGTTTTGTTTCATTTCGTTTGCCAGGCGGGCGGCTTCTTCATTGACCAAAGCAACCGG
AACTTTCAACTCTACGGCTTTAATCAGCGCGTTCATTACGGATTCTTTGGTTTGTTCG
TTCACGCGGCGTTCCACTTCGCGGCTTACGTTTTTCTTCACTTCTTCACGCATTTTGGC
AACATCGCCATCCGCAATACCCAAGGCTTTTGCAAAATCTGCATCGACTTCAGGCAG
AGTGGGCTCGGAAACATTGTTCAGCGTAATGGTGAACACGGCAGTTTTACCGGCAA
CATCTTTGCCGTGGTATTCTTCAGGGAAGTTGACGGTAACGTCTTTACTTTCGCCCGC
TTTCATGCCGACTACGCCGGCTTCAAATTCAGGCAGCATTTGACCTGCGCCCAATAC
GAAGGCGTAGTTTTTGGATGTGCCGCCGGCAAAAGGTTCGCCGTCGATTTTGCCTTC
AAAGTCGATGATGACGCGGTCGCCGTTTCGGGCTTCGCGGTCAACATGGTTGAAGCG
GGTACGTTGTTTACGCAGGATTTCTACAGTTTGGTCGACTTCGGCATCGCCGACGGA
AGCGGTTACTTTTTCGACCTCTTGTGCAGACAAATCGCCGATAACGACTTCGGGGAA
CACTTCAAAAATGGCGGCAACTTTGAAAGACTCTTTATCGTCTTGTTCTTCAACGCCT
TCAAAACGTGGATAGCCTGCCACTTTCAACTCTTGGGCAACGGCAACATCGTAGAAG
CGGCGTTGCACCAGCTCGTTGATCACGTCGTTTTGCGCGCTCGCACCGTACATTTGG
GCAATCATTTTTAAAGGTGCTTTACCCGGACGGAAACCGTCGATTTTTGCACGGCGT
TGGGTTTGTTTCAGTTTTTTATCGGTTTCTGCGTTGATTTCGGACCAAGGCAGGGACA
ACACTACTTTGCGTTCCAGATTTTCTAAAATTTCAACAGTTACGCTCATCAT

RNA: SEQ ID NO: 54

AUGAUGAGCGUAACUGUUGAAAUUUUAGAAAAUCUGGAACGCAAAGUAGUGUUG
UCCCUGCCUUGGUCCGAAAUCAACGCAGAAACCGAUAAAAAACUGAAACAAACCC
AACGCCGUGCAAAAAUCGACGGUUUCCGUCCGGGUAAAGCACCUUUAAAAAUGA
UUGCCCAAAUGUACGGUGCGAGCGCGCAAAACGACGUGAUCAACGAGCUGGUGCA
ACGCCGCUUCUACGAUGUUGCCGUUGCCCAAGAGUUGAAAGUGGCAGGCUAUCCA
CGUUUUGAAGGCGUUGAAGAACAAGACGAUAAAGAGUCUUUCAAAGUUGCCGCC
AUUUUUGAAGUGUUCCCCGAAGUCGUUAUCGGCGAUUUGUCUGCACAAGAGGUC
GAAAAAGUAACCGCUUCCGUCGGCGAUGCCGAAGUCGACCAAACUGUAGAAAUCC
UGCGUAAACAACGUACCCGCUUCAACCAUGUUGACCGCGAAGCCCGAAACGGCGA
CCGCGUCAUCAUCGACUUUGAAGGCAAAAUCGACGGCGAACCUUUUGCCGGCGGC
ACAUCCAAAAACUACGCCUUCGUAUUGGGCGCAGGUCAAAUGCUGCCUGAAUUUG
AAGCCGGCGUAGUCGGCAUGAAAGCGGGCGAAAGUAAAGACGUUACCGUCAACU
UCCCUGAAGAAUACCACGGCAAAGAUGUUGCCGGUAAAACUGCCGUGUUCACCAU
UACGCUGAACAAUGUUUCCGAGCCCACUCUGCCUGAAGUCGAUGCAGAUUUUGCA
AAAGCCUUGGGUAUUGCGGAUGGCGAUGUUGCCAAAAUGCGUGAAGAAGUGAAG
AAAAACGUAAGCCGCGAAGUGGAACGCCGCGUGAACGAACAAACCAAAGAAUCCG
UAAUGAACGCGCUGAUUAAAGCCGUAGAGUUGAAAGUUCCGGUUGCUUUGGUCA
AUGAAGAAGCCGCCCGCCUGGCAAACGAAAUGAAACAAAACUUCGUUAACCAAGG
UAUGACCGAUGCCGCGAACUUGGAUUUGCCUUUGGAUAUGUUCAAAGAACAAGC
CGAACGUCGCGUAUCUUUGGGUCUGAUUUUGGCCAAACUGGUUGACGAAAACAA
ACUGGAACCGACUGAAGGGCAAAUCAAAGCCGUUGUCGCCAACUUCGCAGAAAGC
UACGAAGAUCCUCAAGAAGUGAUUGACUGGUACUACGCAGAUACUUCCCGCCUGC
AAGCCCCGACUUCUUUGGCAGUAGAAAGCAACGUUGUUGAUUUCGUUUUGGGCA
AAGCCAAAGUAAACAAAAAAGCUUUGUCUUUUGACGAAGUGAUGGGCGCGCAAG
CCUGA

### 10. Ngo0593: Nc_002946.2:580181-580795

DNA (+ strand): SEQ ID NO: 55

ATGTCTTTTGATAACCATCTTGTCCCTACCGTTATCGAGCAGAGCGGTCGCGGTGAG
CGTGCATTCGATATCTATTCCCGGCTTTTGAAAGAGCGCATCGTATTTCTGGTTGGCC
CGGTAACCGATGAGTCTGCTAATCTGGTGGTCGCCCAACTGTTGTTTTTGGAAAGTG
AGAATCCGGATAAGGATATTTTCTTCTACATCAATTCCCCCGGCGGCTCGGTAACGG
CCGGTATGTCGATTTACGACACGATGAATTTCATCAAGCCCGATGTATCGACTTTGT
GCTTGGGGCAGGCGGCAAGTATGGGCGCGTTCTTATTGTCGGCAGGCGAGAAAGGC
AAACGTTTCGCCCTGCCCAACAGCCGGATTATGATTCACCAGCCTTTAATCAGCGGC
GGCTTGGGCGGTCAGGCATCCGACATTGAAATCCACGCACGCGAGTTGTTGAAAAT
CAAAGAAAAACTCAACCGCCTGATGGCGAAACATTGCGGCCGCGATTTGGCAGATT
TGGAGCGCGACACCGACCGTGATAATTTCATGTCTGCCGAAGAAGCAAAAGAATAT
GGTTTGATCGACCAAGTTTTGGAAAACCGCGCTTCTTTGCGGCTTTAA

cDNA: SEQ ID NO: 56

TTAAAGCCGCAAAGAAGCGCGGTTTTCCAAAACTTGGTCGATCAAACCATATTCTTT
TGCTTCTTCGGCAGACATGAAATTATCACGGTCGGTGTCGCGCTCCAAATCTGCCAA
ATCGCGGCCGCAATGTTTCGCCATCAGGCGGTTGAGTTTTTCTTTGATTTTCAACAAC
TCGCGTGCGTGGATTTCAATGTCGGATGCCTGACCGCCCAAGCCGCCGCTGATTAAA
GGCTGGTGAATCATAATCCGGCTGTTGGGCAGGGCGAAACGTTTGCCTTTCTCGCCT
GCCGACAATAAGAACGCGCCCATACTTGCCGCCTGCCCCAAGCACAAAGTCGATAC
ATCGGGCTTGATGAAATTCATCGTGTCGTAAATCGACATACCGGCCGTTACCGAGCC
GCCGGGGGAATTGATGTAGAAGAAAATATCCTTATCCGGATTCTCACTTTCCAAAAA
CAACAGTTGGGCGACCACCAGATTAGCAGACTCATCGGTTACCGGGCCAACCAGAA
ATACGATGCGCTCTTTCAAAAGCCGGGAATAGATATCGAATGCACGCTCACCGCGA
CCGCTCTGCTCGATAACGGTAGGGACAAGATGGTTATCAAAAGACAT

RNA: SEQ ID NO: 57

AUGUCUUUUGAUAACCAUCUUGUCCCUACCGUUAUCGAGCAGAGCGGUCGCGGUG
AGCGUGCAUUCGAUAUCUAUUCCCGGCUUUUGAAAGAGCGCAUCGUAUUUCUGG
UUGGCCCGGUAACCGAUGAGUCUGCUAAUCUGGUGGUCGCCCAACUGUUGUUUU
UGGAAAGUGAGAAUCCGGAUAAGGAUAUUUUCUUCUACAUCAAUUCCCCCGGCG
GCUCGGUAACGGCCGGUAUGUCGAUUUACGACACGAUGAAUUUCAUCAAGCCCGA
UGUAUCGACUUUGUGCUUGGGGCAGGCGGCAAGUAUGGGCGCGUUCUUAUUGUC
GGCAGGCGAGAAAGGCAAACGUUUCGCCCUGCCCAACAGCCGGAUUAUGAUUCAC
CAGCCUUUAAUCAGCGGCGGCUUGGGCGGUCAGGCAUCCGACAUUGAAAUCCACG
CACGCGAGUUGUUGAAAAUCAAAGAAAAACUCAACCGCCUGAUGGCGAAACAUU
GCGGCCGCGAUUUGGCAGAUUUGGAGCGCGACACCGACCGUGAUAAUUUCAUGUC
UGCCGAAGAAGCAAAAGAAUAUGGUUUGAUCGACCAAGUUUUGGAAAACCGCGC
UUCUUUGCGGCUUUAA

### 11. NGO0604: NC_002946.2:c591006-589321

DNA (- strand): SEQ ID NO: 58

ATGTCTATGGAAAATTTTGCTCAGCTGTTGGAAGAAAGCTTTACCCTGCAAGAAATG
ACCCGGGTGAGGTGATTACCGCTGAAGTAGTGGCAATCGACCAAAACTTCGTTAC
CGTAAACGCAGGTCTGAAATCAGAATCCCTGATCGATGTAGCTGAATTCAAAAACG
CTCAAGGCGAAATTGAAGTTAAAGTCGGCGACTTCGTTACCGTTACCATCGAATCCG
TCGAAAACGGCTTCGGCGAAACCAAACTGTCCCGCGAAAAAGCCAAACGCGCAGCC
GATTGGATCGCTTTGGAAGAAGCCATGGAAAACGGCAACATCCTGTCCGGCATCAT
CAACGGTAAAGTCAAAGGCGGCCTGACCGTTATGATCAGCAGCATCCGCGCATTCC
TGCCGGGTTCTTTGGTCGACGTACGTCCCGTTAAAGACACTTCCCATTTTGAAGGCA
AAGAGATCGAATTCAAAGTGATCAAACTGGACAAAAAACGCAACAACGTCGTTGTT
TCCCGCCGCGCCGTTTTGGAAGCCACTTTGGGTGAAGAACGCAAAGCCCTGCTGGA
AAACCTGCAAGAAGGCTCCGTCATCAAAGGCATCGTCAAAAATATCACCGACTACG
GCGCATTCGTTGACCTGGGCGGCATCGACGGCCTGCTGCACATCACCGATTTGGCAT
GGCGTCGCGTGAAACACCCGAGCGAAGTCTTGGAAGTCGGTCAGGAAGTTGAAGCC
AAAGTATTGAAATTCGACCAAGAAAAACAACGTGTTTCCTTGGGTATGAAACAACT
GGGCGAAGATCCTTGGAGCGGTCTGACCCGCCGTTATCCGCAAGCCACCCGCCTGTT
CGGCAAAGTATCCAACCTGACCGACTACGGCGCATTCGTCGAAATCGAACAAGGCA
TCGAAGGTTTGGTACACGTCTCCGAAATGGACTGGACCAACAAAAACGTACACCCG
AGCAAAGTCGTACAACTGGGTGACGAAGTCGAAGTCATGATTTTGGAAATCGACGA
AGGCCGCCGCCGTATCTCTTTGGGTATGAAACAATGCCAAGCCAATCCTTGGGAAGA
ATTTGCCGCCAACCACAACAAAGGAGACAAAATCTCCGGTGCGGTTAAATCCATTA
CCGATTTCGGCGTATTCGTCGGCCTGCCCGGCGGCATCGACGGTCTGGTTCACCTGT
CCGACCTGTCTTGGACCGAATCCGGCGAAGAAGCCGTACGCAAATACAAAAAAGGA
GAAGAAGTCGAAGCCGTCGTATTGGCAATCGATGTGGAAAAAGAACGCATCTCCTT
GGGTATCAAACAACTGGAAGGCGATCCTTTCGGCAACTTCATCAGCGTGAACGACA
AAGGTTCTTTGGTTAAAGGTTCCGTGAAATCTGTTGACGCCAAAGGCGCTGTTATCG
CCCTGTCTGACGAAGTAGAAGGCTACCTGCCTGCTTCCGAATTTGCAGCCGACCGCG
TTGAAGACTTGACCACCAAACTGAAAGAAGGCGACGAAGTTGAAGCCGTCATCGTT
ACCGTTGACCGCAAAAACCGCAGCATCAAACTTTCCGTTAAAGCCAAAGATGCCAA
AGAAAGCCGCGAAGCACTGAACTCCGTCAATGCCGCCGCCAATGCGAATGCCGGTA
CCACCAGCTTGGGCGACCTGCTGAAAGCCAAACTCTCCGGCGAACAAGAATAA

cDNA: SEQ ID NO: 59

TTATTCTTGTTCGCCGGAGAGTTTGGCTTTCAGCAGGTCGCCCAAGCTGGTGGTACC
GGCATTCGCATTGGCGGCGGCATTGACGGAGTTCAGTGCTTCGCGGCTTTCTTTGGC
ATCTTTGGCTTTAACGGAAAGTTTGATGCTGCGGTTTTTGCGGTCAACGGTAACGAT
GACGGCTTCAACTTCGTCGCCTTCTTTCAGTTTGGTGGTCAAGTCTTCAACGCGGTCG
GCTGCAAATTCGGAAGCAGGCAGGTAGCCTTCTACTTCGTCAGACAGGGCGATAAC
AGCGCCTTTGGCGTCAACAGATTTCACGGAACCTTTAACCAAAGAACCTTTGTCGTT
CACGCTGATGAAGTTGCCGAAAGGATCGCCTTCCAGTTGTTTGATACCCAAGGAGAT
GCGTTCTTTTTCCACATCGATTGCCAATACGACGGCTTCGACTTCTTCTCCTTTTTTGT
ATTTGCGTACGGCTTCTTCGCCGGATTCGGTCCAAGACAGGTCGGACAGGTGAACCA
GACCGTCGATGCCGCCGGGCAGGCCGACGAATACGCCGAAATCGGTAATGGATTTA
ACCGCACCGGAGATTTTGTCTCCTTTGTTGTGGTTGGCGGCAAATTCTTCCCAAGGAT
TGGCTTGGCATTGTTTCATACCCAAAGAGATACGGCGGCGGCCTTCGTCGATTTCCA
AAATCATGACTTCGACTTCGTCACCCAGTTGTACGACTTTGCTCGGGTGTACGTTTTT
GTTGGTCCAGTCCATTTCGGAGACGTGTACCAAACCTTCGATGCCTTGTTCGATTTCG
ACGAATGCGCCGTAGTCGGTCAGGTTGGATACTTTGCCGAACAGGCGGGTGGCTTGC
GGATAACGGCGGGTCAGACCGCTCCAAGGATCTTCGCCCAGTTGTTTCATACCCAAG
GAAACACGTTGTTTTTCTTGGTCGAATTTCAATACTTTGGCTTCAACTTCCTGACCGA
CTTCCAAGACTTCGCTCGGGTGTTTCACGCGACGCCATGCCAAATCGGTGATGTGCA
GCAGGCCGTCGATGCCGCCCAGGTCAACGAATGCGCCGTAGTCGGTGATATTTTTGA
CGATGCCTTTGATGACGGAGCCTTCTTGCAGGTTTTCCAGCAGGGCTTTGCGTTCTTC
ACCCAAAGTGGCTTCCAAAACGGCGCGGCGGGAAACAACGACGTTGTTGCGTTTTTT
GTCCAGTTTGATCACTTTGAATTCGATCTCTTTGCCTTCAAAATGGGAAGTGTCTTTA
ACGGGACGTACGTCGACCAAAGAACCCGGCAGGAATGCGCGGATGCTGCTGATCAT
AACGGTCAGGCCGCCTTTGACTTTACCGTTGATGATGCCGGACAGGATGTTGCCGTT
TTCCATGGCTTCTTCCAAAGCGATCCAATCGGCTGCGCGTTTGGCTTTTTCGCGGGAC
AGTTTGGTTTCGCCGAAGCCGTTTTCGACGGATTCGATGGTAACGGTAACGAAGTCG
CCGACTTTAACTTCAATTTCGCCTTGAGCGTTTTTGAATTCAGCTACATCGATCAGGG
ATTCTGATTTCAGACCTGCGTTTACGGTAACGAAGTTTTGGTCGATTGCCACTACTTC
AGCGGTAATCACCTCACCCGGGTTCATTTCTTGCAGGGTAAAGCTTTCTTCCAACAG
CTGAGCAAAATTTTCCATAGACAT

RNA: SEQ ID NO: 60

AUGUCUAUGGAAAAUUUUGCUCAGCUGUUGGAAGAAAGCUUUACCCUGCAAGAA
AUGAACCCGGGUGAGGUGAUUACCGCUGAAGUAGUGGCAAUCGACCAAAACUUC
GUUACCGUAAACGCAGGUCUGAAAUCAGAAUCCCUGAUCGAUGUAGCUGAAUUC
AAAAACGCUCAAGGCGAAAUUGAAGUUAAAGUCGGCGACUUCGUUACCGUUACC
AUCGAAUCCGUCGAAAACGGCUUCGGCGAAACCAAACUGUCCCGCGAAAAAGCCA
AACGCGCAGCCGAUUGGAUCGCUUUGGAAGAAGCCAUGGAAAACGGCAACAUCCU
GUCCGGCAUCAUCAACGGUAAAGUCAAAGGCGGCCUGACCGUUAUGAUCAGCAGC
AUCCGCGCAUUCCUGCCGGGUUCUUUGGUCGACGUACGUCCCGUUAAAGACACUU
CCCAUUUUGAAGGCAAAGAGAUCGAAUUCAAAGUGAUCAAACUGGACAAAAAAC
GCAACAACGUCGUUGUUUCCCGCCGCGCCGUUUUGGAAGCCACUUUGGGUGAAGA
ACGCAAAGCCCUGCUGGAAAACCUGCAAGAAGGCUCCGUCAUCAAAGGCAUCGUC
AAAAAUAUCACCGACUACGGCGCAUUCGUUGACCUGGGCGGCAUCGACGGCCUGC
UGCACAUCACCGAUUUGGCAUGGCGUCGCGUGAAACACCCGAGCGAAGUCUUGGA
AGUCGGUCAGGAAGUUGAAGCCAAAGUAUUGAAAUUCGACCAAGAAAAACAACG
UGUUUCCUUGGGUAUGAAACAACUGGGCGAAGAUCCUUGGAGCGGUCUGACCCG
CCGUUAUCCGCAAGCCACCCGCCUGUUCGGCAAAGUAUCCAACCUGACCGACUAC
GGCGCAUUCGUCGAAAUCGAACAAGGCAUCGAAGGUUUGGUACACGUCUCCGAA
AUGGACUGGACCAACAAAAACGUACACCCGAGCAAAGUCGUACAACUGGGUGACG
AAGUCGAAGUCAUGAUUUUGGAAAUCGACGAAGGCCGCCGCCGUAUCUCUUUGG
GUAUGAAACAAUGCCAAGCCAAUCCUUGGGAAGAAUUUGCCGCCAACCACAACAA
AGGAGACAAAAUCUCCGGUGCGGUUAAAUCCAUUACCGAUUUCGGCGUAUUCGU
CGGCCUGCCCGGCGGCAUCGACGGUCUGGUUCACCUGUCCGACCUGUCUUGGACC
GAAUCCGGCGAAGAAGCCGUACGCAAAUACAAAAAAGGAGAAGAAGUCGAAGCC
GUCGUAUUGGCAAUCGAUGUGGAAAAAGAACGCAUCUCCUUGGGUAUCAAACAA
CUGGAAGGCGAUCCUUUCGGCAACUUCAUCAGCGUGAACGACAAAGGUUCUUUG
GUUAAAGGUUCCGUGAAAUCUGUUGACGCCAAAGGCGCUGUUAUCGCCCUGUCU
GACGAAGUAGAAGGCUACCUGCCUGCUUCCGAAUUUGCAGCCGACCGCGUUGAAG
ACUUGACCACCAAACUGAAAGAAGGCGACGAAGUUGAAGCCGUCAUCGUUACCGU
UGACCGCAAAAACCGCAGCAUCAAACUUUCCGUUAAAGCCAAAGAUGCCAAAGAA
AGCCGCGAAGCACUGAACUCCGUCAAUGCCGCCGCCAAUGCGAAUGCCGGUACCA
CCAGCUUGGGCGACCUGCUGAAAGCCAAACUCUCCGGCGAACAAGAAUAA

### 12. NGO0618: NC_002946.2:c606708-606268

DNA (- strand): SEQ ID NO: 61

ATGATGCAGACTTTCCGAAAAATCAGCCTGTATGCCGCAACCTTGTGGCTCGGTATG
CAGATTATGGCAGGTTATATCGCCGCACCGGTGCTGTTCAAAATGCTGCCCAAAATG
CAGGCGGGCGAAATTGCCGGCGTATTGTTCGACATCCTCTCTTGGAGCGGGCTTGCC
GTTTGGGGCACGGTACTGGCTGCCGCCTTTGCCGCCCTAACCCGGCGGCAAACCGCC
CTGCTGCTTTTTTTATTGTCCGCCCTTGCCGCCAACCAATTTTTGGTTACACCCGTTAT
CGAGGCACTGAAATACGGGCATGAAAATTGGCTGTTGTCGGTTGCAGGCGGATCCTT
CGGAATGTGGCACGGTATTTCCAGCATGACTTTCATGGCAACCGCCCTACTTTCAGC
AGTTTTAAGTTGGCGGCTTTCCGGCAAAGAGGCCGTCTGA

cDNA: SEQ ID NO: 62

TCAGACGGCCTCTTTGCCGGAAAGCCGCCAACTTAAAACTGCTGAAAGTAGGGCGG
TTGCCATGAAAGTCATGCTGGAAATACCGTGCCACATTCCGAAGGATCCGCCTGCAA
CCGACAACAGCCAATTTTCATGCCCGTATTTCAGTGCCTCGATAACGGGTGTAACCA
AAAATTGGTTGGCGGCAAGGGCGGACAATAAAAAAAGCAGCAGGGCGGTTTGCCGC
CGGGTTAGGGCGGCAAAGGCGGCAGCCAGTACCGTGCCCCAAACGGCAAGCCCGCT
CCAAGAGAGGATGTCGAACAATACGCCGGCAATTTCGCCCGCCTGCATTTTGGGCA
GCATTTTGAACAGCACCGGTGCGGCGATATAACCTGCCATAATCTGCATACCGAGCC
ACAAGGTTGCGGCATACAGGCTGATTTTTCGGAAAGTCTGCATCAT

RNA: SEQ ID NO: 63

AUGAUGCAGACUUUCCGAAAAAUCAGCCUGUAUGCCGCAACCUUGUGGCUCGGUA
UGCAGAUUAUGGCAGGUUAUAUCGCCGCACCGGUGCUGUUCAAAAUGCUGCCCAA
AAUGCAGGCGGGCGAAAUUGCCGGCGUAUUGUUCGACAUCCUCUCUUGGAGCGG
GCUUGCCGUUUGGGGCACGGUACUGGCUGCCGCCUUUGCCGCCCUAACCCGGCGG
CAAACCGCCCUGCUGCUUUUUUUAUUGUCCGCCCUUGCCGCCAACCAAUUUUUGG
UUACACCCGUUAUCGAGGCACUGAAAUACGGGCAUGAAAAUUGGCUGUUGUCGG
UUGCAGGCGGAUCCUUCGGAAUGUGGCACGGUAUUUCCAGCAUGACUUUCAUGG
CAACCGCCCUACUUUCAGCAGUUUUAAGUUGGCGGCUUUCCGGCAAAGAGGCCGU
CUGA

### 13. NGO0619: NC_002946.2:c607565-606723

DNA (- strand): SEQ ID NO: 64

ATGGATATTAAAATCAACGACATCACCCTCGGCAACAATTCGCCTTTCGTCCTATTC
GGCGGCATCAACGTTTTAGAAGATTTGGATTCCACCCTCCAAACCTGTGCGCATTAC
GTCGAAGTTACCCGCAAACTGGGCATCCCCTATATCTTTAAAGCCTCTTTCGACAAG
GCAAACCGCTCGTCTATCCATTCCTATCGCGGCGTAGGCTTGGAAGAAGGCTTAAAG
ATTTTTGAAAAAGTCAAAGCAGAGTTCGGCATCCCCGTCATTACCGACGTACACGAA
CCCCATCAATGCCAACCCGTCGCCGAAGTGTGCGATGTCATCCAGCTTCCCGCCTTT
CTTGCGCGGCAGACCGATTTGGTGGCCGCAATGGCGGAAACGGGCAATGTTATCAA
CATCAAAAAACCCCAGTTCCTCAGCCCTTCGCAAATGAAAAACATCGTGGAAAAAT
TCCGCGAAGCCGGCAACGGGAAGCTGATTTTATGCGAACGCGGCAGCAGCTTCGGC
TACGACAACCTCGTTGTCGATATGCTCGGTTTCGGCGTGATGAAACAAACCTGCGGC
AACCTGCCGGTTATTTTCGACGTTACCCATTCCCTGCAAACCCGCGATGCCGGTTCT
GCCGCATCCGGCGGTCGTCGCGCACAGGCTTTGGATTTGGCACTTGCAGGCATGGCA
ACCCGCCTTGCCGGCCTGTTCCTCGAATCGCACCCCGATCCGAAACTGGCAAAATGC
GACGGCCCCAGCGCGCTGCCGCTACACCTTTTAGAAAATTTTTTAATCCGCATCAAA
GCATTGGACGATTTAATCAAATCACAACCGATTTTAACAATCGAGTAA

cDNA: SEQ ID NO: 65

TTACTCGATTGTTAAAATCGGTTGTGATTTGATTAAATCGTCCAATGCTTTGATGCGG
ATTAAAAAATTTTCTAAAAGGTGTAGCGGCAGCGCGCTGGGGCCGTCGCATTTTGCC
AGTTTCGGATCGGGGTGCGATTCGAGGAACAGGCCGGCAAGGCGGGTTGCCATGCC
TGCAAGTGCCAAATCCAAAGCCTGTGCGCGACGACCGCCGGATGCGGCAGAACCGG
CATCGCGGGTTTGCAGGGAATGGGTAACGTCGAAAATAACCGGCAGGTTGCCGCAG
GTTTGTTTCATCACGCCGAAACCGAGCATATCGACAACGAGGTTGTCGTAGCCGAAG
CTGCTGCCGCGTTCGCATAAAATCAGCTTCCCGTTGCCGGCTTCGCGGAATTTTTCCA
CGATGTTTTTCATTTGCGAAGGGCTGAGGAACTGGGGTTTTTTGATGTTGATAACATT
GCCCGTTTCCGCCATTGCGGCCACCAAATCGGTCTGCCGCGCAAGAAAGGCGGGAA
GCTGGATGACATCGCACACTTCGGCGACGGGTTGGCATTGATGGGGTTCGTGTACGT
CGGTAATGACGGGGATGCCGAACTCTGCTTTGACTTTTTCAAAAATCTTTAAGCCTT
CTTCCAAGCCTACGCCGCGATAGGAATGGATAGACGAGCGGTTTGCCTTGTCGAAA
GAGGCTTTAAAGATATAGGGGATGCCCAGTTTGCGGGTAACTTCGACGTAATGCGC
ACAGGTTTGGAGGGTGGAATCCAAATCTTCTAAAACGTTGATGCCGCCGAATAGGA
CGAAAGGCGAATTGTTGCCGAGGGTGATGTCGTTGATTTTAATATCCAT

RNA: SEQ ID NO: 66

AUGGAUAUUAAAAUCAACGACAUCACCCUCGGCAACAAUUCGCCUUUCGUCCUAU
UCGGCGGCAUCAACGUUUUAGAAGAUUUGGAUUCCACCCUCCAAACCUGUGCGCA
UUACGUCGAAGUUACCCGCAAACUGGGCAUCCCCUAUAUCUUUAAAGCCUCUUUC
GACAAGGCAAACCGCUCGUCUAUCCAUUCCUAUCGCGGCGUAGGCUUGGAAGAAG
GCUUAAAGAUUUUUGAAAAAGUCAAAGCAGAGUUCGGCAUCCCCGUCAUUACCG
ACGUACACGAACCCCAUCAAUGCCAACCCGUCGCCGAAGUGUGCGAUGUCAUCCA
GCUUCCCGCCUUUCUUGCGCGGCAGACCGAUUUGGUGGCCGCAAUGGCGGAAACG
GGCAAUGUUAUCAACAUCAAAAAACCCCAGUUCCUCAGCCCUUCGCAAAUGAAAA
ACAUCGUGGAAAAAUUCCGCGAAGCCGGCAACGGGAAGCUGAUUUUAUGCGAAC
GCGGCAGCAGCUUCGGCUACGACAACCUCGUUGUCGAUAUGCUCGGUUUCGGCGU
GAUGAAACAAACCUGCGGCAACCUGCCGGUUAUUUUCGACGUUACCCAUUCCCUG
CAAACCCGCGAUGCCGGUUCUGCCGCAUCCGGCGGUCGUCGCGCACAGGCUUUGG
AUUUGGCACUUGCAGGCAUGGCAACCCGCCUUGCCGGCCUGUUCCUCGAAUCGCA
CCCCGAUCCGAAACUGGCAAAAUGCGACGGCCCCAGCGCGCUGCCGCUACACCUU
UUAGAAAAUUUUUUAAUCCGCAUCAAAGCAUUGGACGAUUUAAUCAAAUCACAA
CCGAUUUUAACAAUCGAGUAA

### 14. NGO0620: NC_002946.2:c607970-607587

DNA (- strand): SEQ ID NO: 67

ATGTTCCGTACTATACTTGGCGGAAAAATCCACCGCGCCACCGTAACCGAAGCCGAT
TTAAACTACGTCGGCAGCATTACCGTCGATCAAGACCTGTTAGACGCGGCAGGCATC
TGCCCCAACGAAAAAGTCGCCATCGTCAACAACAACAACGGCGAACGTTTTGAAAC
CTATACCATTGCAGGGAAACGCGGCAGCGGCGTGATTTGCCTGAACGGTGCTGCAG
CCAGGCTGGTACAGAAAGGCGACATCGTCATCATTATGTCTTATATCCAACTTTCCG
AACCGGAAATCGCCGCACACGAACCCAAAGTCGTCTTAGTGGACGGAAACAATAAA
ATCCGCGACATCATCTCCTACGAGCCGCCGCACACCGTACTGTAA

cDNA: SEQ ID NO: 68

TTACAGTACGGTGTGCGGCGGCTCGTAGGAGATGATGTCGCGGATTTTATTGTTTCC
GTCCACTAAGACGACTTTGGGTTCGTGTGCGGCGATTTCCGGTTCGGAAAGTTGGAT
ATAAGACATAATGATGACGATGTCGCCTTTCTGTACCAGCCTGGCTGCAGCACCGTT
CAGGCAAATCACGCCGCTGCCGCGTTTCCCTGCAATGGTATAGGTTTCAAAACGTTC
GCCGTTGTTGTTGTTGACGATGGCGACTTTTTCGTTGGGGCAGATGCCTGCCGCGTCT
AACAGGTCTTGATCGACGGTAATGCTGCCGACGTAGTTTAAATCGGCTTCGGTTACG
GTGGCGCGGTGGATTTTTCCGCCAAGTATAGTACGGAACAT

RNA: SEQ ID NO: 69

AUGUUCCGUACUAUACUUGGCGGAAAAAUCCACCGCGCCACCGUAACCGAAGCCG
AUUUAAACUACGUCGGCAGCAUUACCGUCGAUCAAGACCUGUUAGACGCGGCAGG
CAUCUGCCCCAACGAAAAAGUCGCCAUCGUCAACAACAACAACGGCGAACGUUUU
GAAACCUAUACCAUUGCAGGGAAACGCGGCAGCGGCGUGAUUUGCCUGAACGGU
GCUGCAGCCAGGCUGGUACAGAAAGGCGACAUCGUCAUCAUUAUGUCUUAUAUCC
AACUUUCCGAACCGGAAAUCGCCGCACACGAACCCAAAGUCGUCUUAGUGGACGG
AAACAAUAAAAUCCGCGACAUCAUCUCCUACGAGCCGCCGCACACCGUACUGUAA

### 15. Ngo0648: Nc_002946.2:638163-638717

DNA (+ strand): SEQ ID NO: 70

ATGAAAAAAATCATCGCCTCCGCGCTTATCGCAACATTCGCACTCACCGCCTGCCAA
GACGACACGCAGGCGCGGCTCGAACGGCAGCAGAAACAGATTGAAGCCCTGCAAC
AGCAGCTCGCACAGCAGGCAGACGATACGGTTTACCAACTGACTCCCGAAGCAGTC
AAAGACACCATTCCTGCCCAGGCGCAGGCAAACGGCAACAACGGTCAGCCCGTTAC
CGGCAAAGACGGGCAGCAGTATATTTACGACCAATCGACAGGAAGCTGGCTGCTGC
AAAGCCTGATTGGCGCGGCGGCAGGCGCGTTTATCGGCAACGCGCTGGCAAACAAA
TTCACACGGGCGGGCAACCAAGACAGCCCCGTCGCCCGTCGCGCGCGTGCTGCCTA
CCATCAGTCCGCACGCCCCAATGCGCGCACCAGCAGGGATTTGAACACGCGCAGCC
TCCGTGCAAAACAACAGGCGGCGCAGGCGCAGCGTTACCGCCCGACAACGCGCCCG
CCCGTCAATTACCGCCGTCCCGCTATGCGCGGTTTCGGCAGAAGGCGGTAA

cDNA: SEQ ID NO: 71

TTACCGCCTTCTGCCGAAACCGCGCATAGCGGGACGGCGGTAATTGACGGGCGGGC
GCGTTGTCGGGCGGTAACGCTGCGCCTGCGCCGCCTGTTGTTTTGCACGGAGGCTGC
GCGTGTTCAAATCCCTGCTGGTGCGCGCATTGGGGCGTGCGGACTGATGGTAGGCAG
CACGCGCGCGACGGGCGACGGGGCTGTCTTGGTTGCCCGCCCGTGTGAATTTGTTTG
CCAGCGCGTTGCCGATAAACGCGCCTGCCGCCGCGCCAATCAGGCTTTGCAGCAGC
CAGCTTCCTGTCGATTGGTCGTAAATATACTGCTGCCCGTCTTTGCCGGTAACGGGCT
GACCGTTGTTGCCGTTTGCCTGCGCCTGGGCAGGAATGGTGTCTTTGACTGCTTCGG
GAGTCAGTTGGTAAACCGTATCGTCTGCCTGCTGTGCGAGCTGCTGTTGCAGGGCTT
CAATCTGTTTCTGCTGCCGTTCGAGCCGCGCCTGCGTGTCGTCTTGGCAGGCGGTGA
GTGCGAATGTTGCGATAAGCGCGGAGGCGATGATTTTTTTCAT

RNA: SEQ ID NO: 72

AUGAAAAAAAUCAUCGCCUCCGCGCUUAUCGCAACAUUCGCACUCACCGCCUGCC
AAGACGACACGCAGGCGCGGCUCGAACGGCAGCAGAAACAGAUUGAAGCCCUGCA
ACAGCAGCUCGCACAGCAGGCAGACGAUACGGUUUACCAACUGACUCCCGAAGCA
GUCAAAGACACCAUUCCUGCCCAGGCGCAGGCAAACGGCAACAACGGUCAGCCCG
UUACCGGCAAAGACGGGCAGCAGUAUAUUUACGACCAAUCGACAGGAAGCUGGC
UGCUGCAAAGCCUGAUUGGCGCGGCGGCAGGCGCGUUUAUCGGCAACGCGCUGGC
AAACAAAUUCACACGGGCGGGCAACCAAGACAGCCCCGUCGCCCGUCGCGCGCGU
GCUGCCUACCAUCAGUCCGCACGCCCCAAUGCGCGCACCAGCAGGGAUUUGAACA
CGCGCAGCCUCCGUGCAAAACAACAGGCGGCGCAGGCGCAGCGUUACCGCCCGAC
AACGCGCCCGCCCGUCAAUUACCGCCGUCCCGCUAUGCGCGGUUUCGGCAGAAGG
CGGUAA

### 16. Ngo1291: Nc_002946.2:1246814-1247542

DNA (+ strand): SEQ ID NO: 73

ATGGCAGGCCATAGCAAGTGGGCGAATATCCAGCATAAAAAAGCCCGTCAGGATGC
CAAACGCGGCAAAATCTTCACCCGTTTAATCAAAGAAATCACCGTTGCGGCGCGTAT
GGGCGGCGGCGATCCCGGCGCAAATCCGCGCCTGCGTCTGGCTTTGGAAAAAGCAG
CCGAAAACAATATGCCCAAAGACAATGTGCAACGCGCCATCGACAAAGGTACGGGT
AACTTGGAAGGCGTGGAATACATCGAGTTGCGCTACGAAGGCTACGGCATCGGCGG
CGCAGCTTTGATGGTGGACTGCCTGACCGACAACAAAACCCGCACCGTTGCGGACG
TACGCCACGCATTTACCAAAAACGGCGGCAACTTGGGTACCGACGGCTGCGTGGCG
TTCAACTTCGTGCATCAGGGCTATTTGGTATTCGAACCCGGCGTTGACGAAGACGAG
CTGATGGAAGCGGCTTTGGAAGCCGGTGCGGAAGACGTGGTTACCAACGACGACGG
TTCCATCGAAGTCATTACCGCGCCAAATGATTGGGCGGGCGTAAAATCCGCTTTGGA
GGCGGCAGGTTACAAATCCGTTGACGGCGACGTTACGATGCGCGCCCAAAACGAAA
CCGAACTCTCCGGCGACGATGCCGTCAAAATGCAAAAACTGATTGACGCGCTGGAA
GACTTGGACGACGTGCAAGACGTTTACACTTCCGCCGTATTGAATCTGGACTGA

cDNA: SEQ ID NO: 74

TCAGTCCAGATTCAATACGGCGGAAGTGTAAACGTCTTGCACGTCGTCCAAGTCTTC
CAGCGCGTCAATCAGTTTTTGCATTTTGACGGCATCGTCGCCGGAGAGTTCGGTTTC
GTTTTGGGCGCGCATCGTAACGTCGCCGTCAACGGATTTGTAACCTGCCGCCTCCAA
AGCGGATTTTACGCCCGCCCAATCATTTGGCGCGGTAATGACTTCGATGGAACCGTC
GTCGTTGGTAACCACGTCTTCCGCACCGGCTTCCAAAGCCGCTTCCATCAGCTCGTC
TTCGTCAACGCCGGGTTCGAATACCAAATAGCCCTGATGCACGAAGTTGAACGCCA
CGCAGCCGTCGGTACCCAAGTTGCCGCCGTTTTTGGTAAATGCGTGGCGTACGTCCG
CAACGGTGCGGGTTTTGTTGTCGGTCAGGCAGTCCACCATCAAAGCTGCGCCGCCGA
TGCCGTAGCCTTCGTAGCGCAACTCGATGTATTCCACGCCTTCCAAGTTACCCGTAC
CTTTGTCGATGGCGCGTTGCACATTGTCTTTGGGCATATTGTTTTCGGCTGCTTTTTCC
AAAGCCAGACGCAGGCGCGGATTTGCGCCGGGATCGCCGCCGCCCATACGCGCCGC
AACGGTGATTTCTTTGATTAAACGGGTGAAGATTTTGCCGCGTTTGGCATCCTGACG
GGCTTTTTTATGCTGGATATTCGCCCACTTGCTATGGCCTGCCAT

RNA: SEQ ID NO: 75

AUGGCAGGCCAUAGCAAGUGGGCGAAUAUCCAGCAUAAAAAAGCCCGUCAGGAU
GCCAAACGCGGCAAAAUCUUCACCCGUUUAAUCAAAGAAAUCACCGUUGCGGCGC
GUAUGGGCGGCGGCGAUCCCGGCGCAAAUCCGCGCCUGCGUCUGGCUUUGGAAAA
AGCAGCCGAAAACAAUAUGCCCAAAGACAAUGUGCAACGCGCCAUCGACAAAGGU
ACGGGUAACUUGGAAGGCGUGGAAUACAUCGAGUUGCGCUACGAAGGCUACGGC
AUCGGCGGCGCAGCUUUGAUGGUGGACUGCCUGACCGACAACAAAACCCGCACCG
UUGCGGACGUACGCCACGCAUUUACCAAAAACGGCGGCAACUUGGGUACCGACGG
CUGCGUGGCGUUCAACUUCGUGCAUCAGGGCUAUUUGGUAUUCGAACCCGGCGUU
GACGAAGACGAGCUGAUGGAAGCGGCUUUGGAAGCCGGUGCGGAAGACGUGGUU
ACCAACGACGACGGUUCCAUCGAAGUCAUUACCGCGCCAAAUGAUUGGGCGGGCG
UAAAAUCCGCUUUGGAGGCGGCAGGUUACAAAUCCGUUGACGGCGACGUUACGA
UGCGCGCCCAAAACGAAACCGAACUCUCCGGCGACGAUGCCGUCAAAAUGCAAAA
ACUGAUUGACGCGCUGGAAGACUUGGACGACGUGCAAGACGUUUACACUUCCGCC
GUAUUGAAUCUGGACUGA

### 17. NGO1440: NC_002946.2:c1406345-1405167

DNA (- strand): SEQ ID NO: 76

ATGGCAAAAATGATGAAATGGGCGGCTGTTGCGGCGGTCGCGGCGGCAGCGGTTTG
GGGCGGATGGTCTTATCTGAAGCCCGAACCGCAGGCTGCTTATATTACGGAAACGGT
CAGGCGCGGCGATATCAGCCGGACGGTTTCCGCGACGGGCGAGATTTCGCCGTCCA
ACCTGGTATCGGTCGGCGCGCAGGCTTCGGGGCAGATTAAAAAGCTTTATGTCAAAC
TCGGGCAACAGGTCAAAAAGGGCGATTTGATTGCGGAAATCAATTCGACCACGCAG
ACCAACACGATCGATATGGAAAAATCCAAATTGGAAACGTATCAGGCGAAGCTGGT
GTCGGCACAGATTGCATTGGGCAGCGCGGAGAAGAAATATAAGCGTCAGGCGGCGT
TGTGGAAGGATGATGCGACCTCTAAAGAAGATTTGGAAAGCGCGCAGGATGCGCTT
GCCGCCGCCAAAGCCAATGTTGCCGAGTTGAAGGCTTTAATCAGACAGAGCAAAAT
TTCCATCAATACCGCCGAGTCGGATTTGGGCTACACGCGCATTACCGCGACGATGGA
CGGCACGGTGGTGGCGATTCCCGTGGAAGAGGGGCAGACTGTGAACGCGGCGCAGT
CTACGCCGACGATTGTCCAATTGGCGAATCTGGATATGATGTTGAACAAAATGCAGA
TTGCCGAGGGCGATATTACCAAGGTGAAGGCGGGGCAGGATATTTCGTTTACGATTT
TGTCCGAACCGGATACGCCGATTAAGGCGAAGCTCGACAGCGTCGACCCCGGGCTG
ACCACGATGTCGTCGGGCGGCTACAACAGCAGTACGGATACGGCTTCCAATGCGGT
CTATTATTATGCCCGTTCGTTTGTGCCGAATCCGGACGGCAAACTCGCCACGGGGAT
GACGACGCAGAATACGGTTGAAATCGACGGTGTGAAAAATGTGTTGCTTATTCCGTC
GCTGACCGTGAAAAATCGCGGCGGCAAGGCGTTCGTACGCGTGTTGGGTGCGGACG
GCAAGGCAGTGGAACGCGAAATCCGGACCGGTATGAAAGACAGTATGAATACCGA
AGTGAAAAGCGGGTTGAAAGAGGGGGACAAAGTGGTCATCTCCGAAATAACCGCCG
CCGAGCAGCAGGAAAGCGGCGAACGCGCCCTAGGCGGCCCGCCGCGCCGATAA

cDNA: SEQ ID NO: 77

TTATCGGCGCGGCGGGCCGCCTAGGGCGCGTTCGCCGCTTTCCTGCTGCTCGGCGGC
GGTTATTTCGGAGATGACCACTTTGTCCCCCTCTTTCAACCCGCTTTTCACTTCGGTA
TTCATACTGTCTTTCATACCGGTCCGGATTTCGCGTTCCACTGCCTTGCCGTCCGCAC
CCAACACGCGTACGAACGCCTTGCCGCCGCGATTTTTCACGGTCAGCGACGGAATA
AGCAACACATTTTTCACACCGTCGATTTCAACCGTATTCTGCGTCGTCATCCCCGTGG
CGAGTTTGCCGTCCGGATTCGGCACAAACGAACGGGCATAATAATAGACCGCATTG
GAAGCCGTATCCGTACTGCTGTTGTAGCCGCCCGACGACATCGTGGTCAGCCCGGGG
TCGACGCTGTCGAGCTTCGCCTTAATCGGCGTATCCGGTTCGGACAAAATCGTAAAC
GAAATATCCTGCCCCGCCTTCACCTTGGTAATATCGCCCTCGGCAATCTGCATTTTGT
TCAACATCATATCCAGATTCGCCAATTGGACAATCGTCGGCGTAGACTGCGCCGCGT
TCACAGTCTGCCCCTCTTCCACGGGAATCGCCACCACCGTGCCGTCCATCGTCGCGG
TAATGCGCGTGTAGCCCAAATCCGACTCGGCGGTATTGATGGAAATTTTGCTCTGTC
TGATTAAAGCCTTCAACTCGGCAACATTGGCTTTGGCGGCGGCAAGCGCATCCTGCG
CGCTTTCCAAATCTTCTTTAGAGGTCGCATCATCCTTCCACAACGCCGCCTGACGCTT
ATATTTCTTCTCCGCGCTGCCCAATGCAATCTGTGCCGACACCAGCTTCGCCTGATAC
GTTTCCAATTTGGATTTTTCCATATCGATCGTGTTGGTCTGCGTGGTCGAATTGATTT
CCGCAATCAAATCGCCCTTTTTGACCTGTTGCCCGAGTTTGACATAAAGCTTTTTAAT
CTGCCCCGAAGCCTGCGCGCCGACCGATACCAGGTTGGACGGCGAAATCTCGCCCG
TCGCGGAAACCGTCCGGCTGATATCGCCGCGCCTGACCGTTTCCGTAATATAAGCAG
CCTGCGGTTCGGGCTTCAGATAAGACCATCCGCCCCAAACCGCTGCCGCCGCGACCG
CCGCAACAGCCGCCCATTTCATCATTTTTGCCAT

RNA: SEQ ID NO: 78

AUGGCAAAAAUGAUGAAAUGGGCGGCUGUUGCGGCGGUCGCGGCGGCAGCGGUU
UGGGGCGGAUGGUCUUAUCUGAAGCCCGAACCGCAGGCUGCUUAUAUUACGGAA
ACGGUCAGGCGCGGCGAUAUCAGCCGGACGGUUUCCGCGACGGGCGAGAUUUCGC
CGUCCAACCUGGUAUCGGUCGGCGCGCAGGCUUCGGGGCAGAUUAAAAAGCUUUA
UGUCAAACUCGGGCAACAGGUCAAAAAGGGCGAUUUGAUUGCGGAAAUCAAUUC
GACCACGCAGACCAACACGAUCGAUAUGGAAAAAUCCAAAUUGGAAACGUAUCA
GGCGAAGCUGGUGUCGGCACAGAUUGCAUUGGGCAGCGCGGAGAAGAAAUAUAA
GCGUCAGGCGGCGUUGUGGAAGGAUGAUGCGACCUCUAAAGAAGAUUUGGAAAG
CGCGCAGGAUGCGCUUGCCGCCGCCAAAGCCAAUGUUGCCGAGUUGAAGGCUUUA
AUCAGACAGAGCAAAAUUUCCAUCAAUACCGCCGAGUCGGAUUUGGGCUACACGC
GCAUUACCGCGACGAUGGACGGCACGGUGGUGGCGAUUCCCGUGGAAGAGGGGC
AGACUGUGAACGCGGCGCAGUCUACGCCGACGAUUGUCCAAUUGGCGAAUCUGGA
UAUGAUGUUGAACAAAAUGCAGAUUGCCGAGGGCGAUAUUACCAAGGUGAAGGC
GGGGCAGGAUAUUUCGUUUACGAUUUUGUCCGAACCGGAUACGCCGAUUAAGGC
GAAGCUCGACAGCGUCGACCCCGGGCUGACCACGAUGUCGUCGGGCGGCUACAAC
AGCAGUACGGAUACGGCUUCCAAUGCGGUCUAUUAUUAUGCCCGUUCGUUUGUG
CCGAAUCCGGACGGCAAACUCGCCACGGGGAUGACGACGCAGAAUACGGUUGAAA
UCGACGGUGUGAAAAAUGUGUUGCUUAUUCCGUCGCUGACCGUGAAAAAUCGCG
GCGGCAAGGCGUUCGUACGCGUGUUGGGUGCGGACGGCAAGGCAGUGGAACGCG
AAAUCCGGACCGGUAUGAAAGACAGUAUGAAUACCGAAGUGAAAAGCGGGUUGA
AAGAGGGGGACAAAGUGGUCAUCUCCGAAAUAACCGCCGCCGAGCAGCAGGAAA
GCGGCGAACGCGCCCUAGGCGGCCCGCCGCGCCGAUAA

### 18. NGO1658: NC_002946.2:c1613531-1613241

DNA (- strand): SEQ ID NO: 79

GTGGAATATTTTATGTTGCTGGCAACAGACGGGGAGGATGTGCATGAAGCGCGTAT
GGCGGCACGTCCCGAACACTTTAAACGGCTGGAAACGCTGAAATCGGAAGGCCGTC
TGCTGACGGCAGGCCCAAACCTGCTGCCGGACAATCCCGAACGTGTTTCGGGCAGC
TTGATTGTGGCACAGTTCGAGTCTTTGGATGCGGCGCAGGCTTGGGCTGAAGACGAT
CCCTATGTTCATGCCGGCGTGTACAGCGAAGTGCTGATCAAGCCGTTTAAAGCGGTG
TTCAAATAA

cDNA: SEQ ID NO: 80

TTATTTGAACACCGCTTTAAACGGCTTGATCAGCACTTCGCTGTACACGCCGGCATG
AACATAGGGATCGTCTTCAGCCCAAGCCTGCGCCGCATCCAAAGACTCGAACTGTG
CCACAATCAAGCTGCCCGAAACACGTTCGGGATTGTCCGGCAGCAGGTTTGGGCCT
GCCGTCAGCAGACGGCCTTCCGATTTCAGCGTTTCCAGCCGTTTAAAGTGTTCGGGA
CGTGCCGCCATACGCGCTTCATGCACATCCTCCCCGTCTGTTGCCAGCAACATAAAA
TATTCCAC

RNA: SEQ ID NO: 81

GUGGAAUAUUUUAUGUUGCUGGCAACAGACGGGGAGGAUGUGCAUGAAGCGCGU
AUGGCGGCACGUCCCGAACACUUUAAACGGCUGGAAACGCUGAAAUCGGAAGGCC
GUCUGCUGACGGCAGGCCCAAACCUGCUGCCGGACAAUCCCGAACGUGUUUCGGG
CAGCUUGAUUGUGGCACAGUUCGAGUCUUUGGAUGCGGCGCAGGCUUGGGCUGA
AGACGAUCCCUAUGUUCAUGCCGGCGUGUACAGCGAAGUGCUGAUCAAGCCGUUU
AAAGCGGUGUUCAAAUAA

### 19. NGO1659: NC_002946.2:c1614064-1613534

DNA (- strand): SEQ ID NO: 82

ATGAAATTTGTCAGCGACCTTTTGTCCGTCATCTTGTTTTTTGCTACTTATACCGTTAC
CAAAAATATGATTGCCGCTGCGGCGGTTGCCTTGGTTGCAGGCGTGGTTCAGGCGGC
TTTCCTGTATTGGAAGCATAAAAGGCTGGATACGATGCAGTGGGTCGGACTGGTGCT
GATTGTCGTATTCGGCGGCGCAACCATTGTTTTGGGCGACAGCCGCTTCATTATGTG
GAAGCCGACAGTATTGTTCTGGTGCGGGGCGTTATTCCTGCTGGGCAGCCACCTTGC
GGGTAAAAACGGCTTGAAAGCGAGTATCGGCAGGGAGATTCAGCTTCCGGATGCCG
TATGGGGAAAATTGACATATATGTGGGTCGGTTTTCTGATTTTTATGGGTATTGCCA
ACTGGTTTGTGTTTACTAGGTTTGAAGCGCAATGGGTTAACTATAAGATGTTCGGTT
CGACTGCGCTGATGCTTTTTTTCTTTATTATTCAGGGTATTTATCTGAGTACCTATCTG
AAAAAGGAGGATTGA

cDNA: SEQ ID NO: 83

TCAATCCTCCTTTTTCAGATAGGTACTCAGATAAATACCCTGAATAATAAAGAAAAA
AAGCATCAGCGCAGTCGAACCGAACATCTTATAGTTAACCCATTGCGCTTCAAACCT
AGTAAACACAAACCAGTTGGCAATACCCATAAAAATCAGAAAACCGACCCACATAT
ATGTCAATTTTCCCCATACGGCATCCGGAAGCTGAATCTCCCTGCCGATACTCGCTTT
CAAGCCGTTTTTACCCGCAAGGTGGCTGCCCAGCAGGAATAACGCCCCGCACCAGA
ACAATACTGTCGGCTTCCACATAATGAAGCGGCTGTCGCCCAAAACAATGGTTGCGC
CGCCGAATACGACAATCAGCACCAGTCCGACCCACTGCATCGTATCCAGCCTTTTAT
GCTTCCAATACAGGAAAGCCGCCTGAACCACGCCTGCAACCAAGGCAACCGCCGCA
GCGGCAATCATATTTTTGGTAACGGTATAAGTAGCAAAAAACAAGATGACGGACAA
AAGGTCGCTGACAAATTTCAT

RNA: SEQ ID NO: 84

AUGAAAUUUGUCAGCGACCUUUUGUCCGUCAUCUUGUUUUUUGCUACUUAUACC
GUUACCAAAAAUAUGAUUGCCGCUGCGGCGGUUGCCUUGGUUGCAGGCGUGGUU
CAGGCGGCUUUCCUGUAUUGGAAGCAUAAAAGGCUGGAUACGAUGCAGUGGGUC
GGACUGGUGCUGAUUGUCGUAUUCGGCGGCGCAACCAUUGUUUUGGGCGACAGC
CGCUUCAUUAUGUGGAAGCCGACAGUAUUGUUCUGGUGCGGGGCGUUAUUCCUG
CUGGGCAGCCACCUUGCGGGUAAAAACGGCUUGAAAGCGAGUAUCGGCAGGGAG
AUUCAGCUUCCGGAUGCCGUAUGGGGAAAAUUGACAUAUAUGUGGGUCGGUUUU
CUGAUUUUUAUGGGUAUUGCCAACUGGUUUGUGUUUACUAGGUUUGAAGCGCAA
UGGGUUAACUAUAAGAUGUUCGGUUCGACUGCGCUGAUGCUUUUUUUCUUUAUU
AUUCAGGGUAUUUAUCUGAGUACCUAUCUGAAAAAGGAGGAUUGA

### 20. NGO1673: NC_002946.2:c1629235-1627559

DNA (- strand): SEQ ID NO: 85

ATGAGCGTAGGTTTGCTGAGGATTCTGGTTCAAAACCAGGTGGTTACTGTTGAGCGG
GCCGAGCATTACTACAATGAGTCGCAGGCGGGTAAGGAAGTGTTGCCGATGCTGTTT
TCAGACGGTGTCATTTCGCCCAAGTCGCTTGCGGCATTGATTGCGAGGGTGTTCAGT
TATTCGATTCTTGATTTGCGTCATTATCCGCGCCACAGGGTGCTGATGGGGGTGTTG
ACGGAGGAGCAGATGGTGGAGTTCCACTGTGTGCCGGTTTTCCGTCGGGGCGACAA
AGTATTTTTTGCGGTTTCCGATCCGACCCAGATGCCGCAAATTCAGAAAACCGTTTC
TGCCGCAGGGATTGCGGTTGAGTTGGTCATTGTCGAGGATGACCAGTTGGCGGGTTT
GCTCGATTGGGTGGGTTCGCGTTCGACATCGCTGCTTCAGGAGCTTGGGGAGGGGCA
AGAGGAAGAGGAAAGCCACACCCTGTATATCGACAACGAGGAGGCAGAAGACGGC
CCTGTTCCGAGGTTTATCCATAAAACTTTGTCGGATGCCTTGCGTAGCGGGGCATCC
GACATCCATTTCGAGTTTTACGAACACAATGCGCGTATCCGTTTCCGTGTGGACGGG
CAGCTCCGCGAGGTGGTTCAGCCGCCCATTGCGGTAAGGGGGCAGCTTGCTTCCCGG
ATTAAGGTAATGTCGCGTTTGGACATTTCCGAAAAACGGATACCGCAGGACGGTAG
GATGCAGCTGACCTTTCAAAAGGGCGGCAAGCCTGTCGATTTCCGTGTCAGCACATT
GCCGACGCTGTTTGGCGAAAAGGTCGTGATGCGGATTTTGAATTCCGATGCCGCGTC
TTTGAACATCGACCAGCTCGGTTTTGAGCCGTTCCAGAAAAAATTGTTGTTGGAAGC
GATTCACCGTCCTTACGGGATGGTGCTGGTAACCGGTCCGACGGGTTCGGGTAAGAC
GGTGTCGCTCTATACCTGTTTGAATATTTTGAATACGGAGTCGGTAAATATTGCAAC
GGCGGAAGACCCTGCCGAGATTAACCTGCCGGGCATCAATCAGGTTAACGTCAATG
ATAAGCAGGGTCTGACTTTTGCCGCTGCTTTGAAGTCTTTCCTGCGTCAGGACCCGG
ACATCATTATGGTCGGTGAGATTCGTGATTTGGAAACTGCCGATATTGCGATTAAGG
CGGCACAAACAGGGCATATGGTGTTTTCCACACTGCACACGAATAATGCGCCGGCG
ACGTTGTCGCGTATGCTGAATATGGGTGTCGCGCCGTTTAATATTGCCAGTTCGGTC
AGCCTGATTATGGCGCAGCGTCTTTTACGCAGGCTGTGTTCGAGCTGCAAACAGGAA
GTGGAACGCCCGTCTGCCTCTGCTTTGAAGGAAGTCGGTTTCACCGATGAGGATCTT
GCAAAAGATTGGAAACTTTACCGCGCCGTCGGTTGCGACCGTTGCCGGGGGCAGGG
TTATAAGGGGCGTGCGGGCGTGTATGAGGTTATGCCCATCAGCGAAGAAATGCAGC
GTGTGATTATGAACAACGGTACGGAAGTGGGTATTTTGGACGTTGCCTATAAGGAG
GGTATGGTGGATTTGCGCCGGGCCGGTATTTTGAAAATTATGCAGGGCATTACTTCA
TTGGAAGAGGTAACGGCAAATACCAACGATTAG

cDNA: SEQ ID NO: 86

CTAATCGTTGGTATTTGCCGTTACCTCTTCCAATGAAGTAATGCCCTGCATAATTTTC
AAAATACCGGCCCGGCGCAAATCCACCATACCCTCCTTATAGGCAACGTCCAAAAT
ACCCACTTCCGTACCGTTGTTCATAATCACACGCTGCATTTCTTCGCTGATGGGCATA
ACCTCATACACGCCCGCACGCCCCTTATAACCCTGCCCCCGGCAACGGTCGCAACCG
ACGGCGCGGTAAAGTTTCCAATCTTTTGCAAGATCCTCATCGGTGAAACCGACTTCC
TTCAAAGCAGAGGCAGACGGGCGTTCCACTTCCTGTTTGCAGCTCGAACACAGCCTG
CGTAAAAGACGCTGCGCCATAATCAGGCTGACCGAACTGGCAATATTAAACGGCGC
GACACCCATATTCAGCATACGCGACAACGTCGCCGGCGCATTATTCGTGTGCAGTGT
GGAAAACACCATATGCCCTGTTTGTGCCGCCTTAATCGCAATATCGGCAGTTTCCAA
ATCACGAATCTCACCGACCATAATGATGTCCGGGTCCTGACGCAGGAAAGACTTCA
AAGCAGCGGCAAAAGTCAGACCCTGCTTATCATTGACGTTAACCTGATTGATGCCCG
GCAGGTTAATCTCGGCAGGGTCTTCCGCCGTTGCAATATTTACCGACTCCGTATTCA
AAATATTCAAACAGGTATAGAGCGACACCGTCTTACCCGAACCCGTCGGACCGGTT
ACCAGCACCATCCCGTAAGGACGGTGAATCGCTTCCAACAACAATTTTTTCTGGAAC
GGCTCAAAACCGAGCTGGTCGATGTTCAAAGACGCGGCATCGGAATTCAAAATCCG
CATCACGACCTTTTCGCCAAACAGCGTCGGCAATGTGCTGACACGGAAATCGACAG
GCTTGCCGCCCTTTTGAAAGGTCAGCTGCATCCTACCGTCCTGCGGTATCCGTTTTTC
GGAAATGTCCAAACGCGACATTACCTTAATCCGGGAAGCAAGCTGCCCCCTTACCG
CAATGGGCGGCTGAACCACCTCGCGGAGCTGCCCGTCCACACGGAAACGGATACGC
GCATTGTGTTCGTAAAACTCGAAATGGATGTCGGATGCCCCGCTACGCAAGGCATCC
GACAAAGTTTTATGGATAAACCTCGGAACAGGGCCGTCTTCTGCCTCCTCGTTGTCG
ATATACAGGGTGTGGCTTTCCTCTTCCTCTTGCCCCTCCCCAAGCTCCTGAAGCAGCG
ATGTCGAACGCGAACCCACCCAATCGAGCAAACCCGCCAACTGGTCATCCTCGACA
ATGACCAACTCAACCGCAATCCCTGCGGCAGAAACGGTTTTCTGAATTTGCGGCATC
TGGGTCGGATCGGAAACCGCAAAAAATACTTTGTCGCCCCGACGGAAAACCGGCAC
ACAGTGGAACTCCACCATCTGCTCCTCCGTCAACACCCCCATCAGCACCCTGTGGCG
CGGATAATGACGCAAATCAAGAATCGAATAACTGAACACCCTCGCAATCAATGCCG
CAAGCGACTTGGGCGAAATGACACCGTCTGAAAACAGCATCGGCAACACTTCCTTA
CCCGCCTGCGACTCATTGTAGTAATGCTCGGCCCGCTCAACAGTAACCACCTGGTTT
TGAACCAGAATCCTCAGCAAACCTACGCTCAT

RNA: SEQ ID NO: 87

AUGAGCGUAGGUUUGCUGAGGAUUCUGGUUCAAAACCAGGUGGUUACUGUUGAG
CGGGCCGAGCAUUACUACAAUGAGUCGCAGGCGGGUAAGGAAGUGUUGCCGAUG
CUGUUUUCAGACGGUGUCAUUUCGCCCAAGUCGCUUGCGGCAUUGAUUGCGAGG
GUGUUCAGUUAUUCGAUUCUUGAUUUGCGUCAUUAUCCGCGCCACAGGGUGCUG
AUGGGGGUGUUGACGGAGGAGCAGAUGGUGGAGUUCCACUGUGUGCCGGUUUUC
CGUCGGGGCGACAAAGUAUUUUUUGCGGUUUCCGAUCCGACCCAGAUGCCGCAAA
UUCAGAAAACCGUUUCUGCCGCAGGGAUUGCGGUUGAGUUGGUCAUUGUCGAGG
AUGACCAGUUGGCGGGUUUGCUCGAUUGGGUGGGUUCGCGUUCGACAUCGCUGC
UUCAGGAGCUUGGGGAGGGGCAAGAGGAAGAGGAAAGCCACACCCUGUAUAUCG
ACAACGAGGAGGCAGAAGACGGCCCUGUUCCGAGGUUUAUCCAUAAAACUUUGU
CGGAUGCCUUGCGUAGCGGGGCAUCCGACAUCCAUUUCGAGUUUUACGAACACAA
UGCGCGUAUCCGUUUCCGUGUGGACGGGCAGCUCCGCGAGGUGGUUCAGCCGCCC
AUUGCGGUAAGGGGGCAGCUUGCUUCCCGGAUUAAGGUAAUGUCGCGUUUGGAC
AUUUCCGAAAAACGGAUACCGCAGGACGGUAGGAUGCAGCUGACCUUUCAAAAG
GGCGGCAAGCCUGUCGAUUUCCGUGUCAGCACAUUGCCGACGCUGUUUGGCGAAA
AGGUCGUGAUGCGGAUUUUGAAUUCCGAUGCCGCGUCUUUGAACAUCGACCAGC
UCGGUUUUGAGCCGUUCCAGAAAAAAUUGUUGUUGGAAGCGAUUCACCGUCCUU
ACGGGAUGGUGCUGGUAACCGGUCCGACGGGUUCGGGUAAGACGGUGUCGCUCU
AUACCUGUUUGAAUAUUUUGAAUACGGAGUCGGUAAAUAUUGCAACGGCGGAAG
ACCCUGCCGAGAUUAACCUGCCGGGCAUCAAUCAGGUUAACGUCAAUGAUAAGCA
GGGUCUGACUUUUGCCGCUGCUUUGAAGUCUUUCCUGCGUCAGGACCCGGACAUC
AUUAUGGUCGGUGAGAUUCGUGAUUUGGAAACUGCCGAUAUUGCGAUUAAGGCG
GCACAAACAGGGCAUAUGGUGUUUUCCACACUGCACACGAAUAAUGCGCCGGCGA
CGUUGUCGCGUAUGCUGAAUAUGGGUGUCGCGCCGUUUAAUAUUGCCAGUUCGG
UCAGCCUGAUUAUGGCGCAGCGUCUUUUACGCAGGCUGUGUUCGAGCUGCAAACA
GGAAGUGGAACGCCCGUCUGCCUCUGCUUUGAAGGAAGUCGGUUUCACCGAUGA
GGAUCUUGCAAAAGAUUGGAAACUUUACCGCGCCGUCGGUUGCGACCGUUGCCGG
GGGCAGGGUUAUAAGGGGCGUGCGGGCGUGUAUGAGGUUAUGCCCAUCAGCGAA
GAAAUGCAGCGUGUGAUUAUGAACAACGGUACGGAAGUGGGUAUUUUGGACGUU
GCCUAUAAGGAGGGUAUGGUGGAUUUGCGCCGGGCCGGUAUUUUGAAAAUUAUG
CAGGGCAUUACUUCAUUGGAAGAGGUAACGGCAAAUACCAACGAUUAG

### 21. Ngo1676: Nc_002946.2:1631221-1631529

DNA (+ strand): SEQ ID NO: 88

ATGTACGCGGTCGTAAAAACCGGCGGCAAACAGTATAAAGTTTCCGTCGGCGAAAA
ATTGAAAGTAGAACAGATACCAGCCCAACTCGACAGCCAAATCGAACTGACCGAAG
TTTTGATGATTGCTGACGGCGAATCTGTAAAAGTTGGCGCACCCTTTATCGAAGGTG
CAAAAGTAACGGCTAAAGTAGTGGCACACGGTCGTGGCGAAAAAGTCCGCATCTTC
AAAATGCGCCGCCGCAAACACTACCAAAAACGCCAAGGCCACCGCCAAAATTTCAC
CCAAATCGAAATCGTGGCAATCGCCTAA

cDNA: SEQ ID NO: 89

TTAGGCGATTGCCACGATTTCGATTTGGGTGAAATTTTGGCGGTGGCCTTGGCGTTTT
TGGTAGTGTTTGCGGCGGCGCATTTTGAAGATGCGGACTTTTTCGCCACGACCGTGT
GCCACTACTTTAGCCGTTACTTTTGCACCTTCGATAAAGGGTGCGCCAACTTTTACAG
ATTCGCCGTCAGCAATCATCAAAACTTCGGTCAGTTCGATTTGGCTGTCGAGTTGGG
CTGGTATCTGTTCTACTTTCAATTTTTCGCCGACGGAAACTTTATACTGTTTGCCGCC
GGTTTTTACGACCGCGTACAT

RNA: SEQ ID NO: 90

AUGUACGCGGUCGUAAAAACCGGCGGCAAACAGUAUAAAGUUUCCGUCGGCGAA
AAAUUGAAAGUAGAACAGAUACCAGCCCAACUCGACAGCCAAAUCGAACUGACCG
AAGUUUUGAUGAUUGCUGACGGCGAAUCUGUAAAAGUUGGCGCACCCUUUAUCG
AAGGUGCAAAAGUAACGGCUAAAGUAGUGGCACACGGUCGUGGCGAAAAAGUCC
GCAUCUUCAAAAUGCGCCGCCGCAAACACUACCAAAAACGCCAAGGCCACCGCCA
AAAUUUCACCCAAAUCGAAAUCGUGGCAAUCGCCUAA

### 22. Ngo1677: Nc_002946.2:1631554-1631826

DNA (+ strand): SEQ ID NO: 91

ATGGCAAGTAAAAAAGCAGGCGGCAGCACCCGCAACGGTCGCGATTCAGAAGCCA
AACGCTTGGGCGTTAAAGCCTACGGCAACGAGCTGATTCCCGCAGGTTCCATCATCG
TACGCCAACGCGGTACCAAATTCCACGCAGGCGACAACGTAGGTATGGGCAAAGAC
CACACTTTGTTCGCCAAAATTGACGGTTATGTCGAATTCAAAACCAAAGGCGCGCTG
AACCGTAAAACTGTCAGCATCCGTCCTTACACCGGTTCTGAAGAATAA

cDNA: SEQ ID NO: 92

TTATTCTTCAGAACCGGTGTAAGGACGGATGCTGACAGTTTTACGGTTCAGCGCGCC
TTTGGTTTTGAATTCGACATAACCGTCAATTTTGGCGAACAAAGTGTGGTCTTTGCCC
ATACCTACGTTGTCGCCTGCGTGGAATTTGGTACCGCGTTGGCGTACGATGATGGAA
CCTGCGGGAATCAGCTCGTTGCCGTAGGCTTTAACGCCCAAGCGTTTGGCTTCTGAA
TCGCGACCGTTGCGGGTGCTGCCGCCTGCTTTTTTACTTGCCAT

RNA: SEQ ID NO: 93

AUGGCAAGUAAAAAAGCAGGCGGCAGCACCCGCAACGGUCGCGAUUCAGAAGCCA
AACGCUUGGGCGUUAAAGCCUACGGCAACGAGCUGAUUCCCGCAGGUUCCAUCAU
CGUACGCCAACGCGGUACCAAAUUCCACGCAGGCGACAACGUAGGUAUGGGCAAA
GACCACACUUUGUUCGCCAAAAUUGACGGUUAUGUCGAAUUCAAAACCAAAGGC
GCGCUGAACCGUAAAACUGUCAGCAUCCGUCCUUACACCGGUUCUGAAGAAUAA

### 23. NGO1679: NC_002946.2:c1633589-1633434

DNA (- strand): SEQ ID NO: 94

ATGCGCGATAAAATCAAACTGGAATCCGGTGCAGGTACTGGCCACTTCTACACCACT
ACCAAAAATAAACGCACTATGCCCGGCAAACTGGAAATCAAAAAATTCGATCCGGT
TGCCCGCAAACACGTAGTGTACAAAGAAACCAAACTGAAATAA

cDNA: SEQ ID NO: 95

TTATTTCAGTTTGGTTTCTTTGTACACTACGTGTTTGCGGGCAACCGGATCGAATTTT
TTGATTTCCAGTTTGCCGGGCATAGTGCGTTTATTTTTGGTAGTGGTGTAGAAGTGGC
CAGTACCTGCACCGGATTCCAGTTTGATTTTATCGCGCAT

RNA: SEQ ID NO: 96

AUGCGCGAUAAAAUCAAACUGGAAUCCGGUGCAGGUACUGGCCACUUCUACACCA
CUACCAAAAAUAAACGCACUAUGCCCGGCAAACUGGAAAUCAAAAAAUUCGAUCC
GGUUGCCCGCAAACACGUAGUGUACAAAGAAACCAAACUGAAAUAA

### 24. Ngo1804: Nc_002946.2:1777677-1778126

DNA (+ strand): SEQ ID NO: 97

ATGGACGTACAACTCCCCATCGAAGCCAAAGACATCCAAAAACTCATCCCCCACCG
CTACCCGTTTCTCCAGCTCGACCGCATTACCGCCTTCGAGCCGATGAAAACCCTGAC
CGCCATCAAAAACGTAACCATAAACGAACCCCAATTCCAAGGCCATTTCCCCGACCT
GCCCGTTATGCCCGGCGTACTCATCATCGAAGCGATGGCGCAGGCGTGCGGCACGTT
GGCGATTTTGAGCGAAGGCGGGCGCAAGGAAAACGAATTTTTCTTCTTCGCCGGCAT
AGACGAAGCCCGTTTCAAACGCCAAGTCATCCCCGGCGACCAACTCGTCTTTGAAGT
CGAACTCCTGACCAGCCGGCGCGGCATCGGCAAATTCAACGCCGTTGCCAAAGTGG
ACGGACAAGTCGCCGTCGAAGCCGTGATTATGTGCGCCAAACGCGTGGTTTGA

cDNA: SEQ ID NO: 98

TCAAACCACGCGTTTGGCGCACATAATCACGGCTTCGACGGCGACTTGTCCGTCCAC
TTTGGCAACGGCGTTGAATTTGCCGATGCCGCGCCGGCTGGTCAGGAGTTCGACTTC
AAAGACGAGTTGGTCGCCGGGGATGACTTGGCGTTTGAAACGGGCTTCGTCTATGCC
GGCGAAGAAGAAAAATTCGTTTTCCTTGCGCCCGCCTTCGCTCAAAATCGCCAACGT
GCCGCACGCCTGCGCCATCGCTTCGATGATGAGTACGCCGGGCATAACGGGCAGGT
CGGGGAAATGGCCTTGGAATTGGGGTTCGTTTATGGTTACGTTTTTGATGGCGGTCA
GGGTTTTCATCGGCTCGAAGGCGGTAATGCGGTCGAGCTGGAGAAACGGGTAGCGG
TGGGGGATGAGTTTTTGGATGTCTTTGGCTTCGATGGGGAGTTGTACGTCCAT

RNA: SEQ ID NO: 99

AUGGACGUACAACUCCCCAUCGAAGCCAAAGACAUCCAAAAACUCAUCCCCCACC
GCUACCCGUUUCUCCAGCUCGACCGCAUUACCGCCUUCGAGCCGAUGAAAACCCU
GACCGCCAUCAAAAACGUAACCAUAAACGAACCCCAAUUCCAAGGCCAUUUCCCC
GACCUGCCCGUUAUGCCCGGCGUACUCAUCAUCGAAGCGAUGGCGCAGGCGUGCG
GCACGUUGGCGAUUUUGAGCGAAGGCGGGCGCAAGGAAAACGAAUUUUUCUUCU
UCGCCGGCAUAGACGAAGCCCGUUUCAAACGCCAAGUCAUCCCCGGCGACCAACU
CGUCUUUGAAGUCGAACUCCUGACCAGCCGGCGCGGCAUCGGCAAAUUCAACGCC
GUUGCCAAAGUGGACGGACAAGUCGCCGUCGAAGCCGUGAUUAUGUGCGCCAAAC
GCGUGGUUUGA

### 25. NGO1833: NC_002946.2:c1803866-1803537

DNA (- strand): SEQ ID NO: 100

ATGAGAGTAAATGCACAACATAAAAATGCCCGTATCTCTGCTCAAAAGGCTCGTTTG
GTAGCTGATTTGATTCGTGGTAAAGACGTTGCCCAAGCTTTGAATATTTTGGCTTTCA
GCCCTAAAAAAGGTGCCGAGCTGATCAAAAAAGTATTGGAGTCAGCTATTGCTAAT
GCTGAGCATAATAACGGTGCGGACATTGATGAACTGAAAGTGGTAACTATCTTTGTT
GACAAAGGTCCAAGCTTGAAACGTTTTCAAGCTCGCGCCAAAGGTCGCGGTAACCG
CATCGAAAAACAAACTTGTCATATCAATGTGACAGTGGGTAACTAA

cDNA: SEQ ID NO: 101

TTAGTTACCCACTGTCACATTGATATGACAAGTTTGTTTTTCGATGCGGTTACCGCGA
CCTTTGGCGCGAGCTTGAAAACGTTTCAAGCTTGGACCTTTGTCAACAAAGATAGTT
ACCACTTTCAGTTCATCAATGTCCGCACCGTTATTATGCTCAGCATTAGCAATAGCTG
ACTCCAATACTTTTTTGATCAGCTCGGCACCTTTTTTAGGGCTGAAAGCCAAAATATT
CAAAGCTTGGGCAACGTCTTTACCACGAATCAAATCAGCTACCAAACGAGCCTTTTG
AGCAGAGATACGGGCATTTTTATGTTGTGCATTTACTCTCAT

RNA: SEQ ID NO: 102

AUGAGAGUAAAUGCACAACAUAAAAAUGCCCGUAUCUCUGCUCAAAAGGCUCGU
UUGGUAGCUGAUUUGAUUCGUGGUAAAGACGUUGCCCAAGCUUUGAAUAUUUUG
GCUUUCAGCCCUAAAAAAGGUGCCGAGCUGAUCAAAAAAGUAUUGGAGUCAGCU
AUUGCUAAUGCUGAGCAUAAUAACGGUGCGGACAUUGAUGAACUGAAAGUGGUA
ACUAUCUUUGUUGACAAAGGUCCAAGCUUGAAACGUUUUCAAGCUCGCGCCAAA
GGUCGCGGUAACCGCAUCGAAAAACAAACUUGUCAUAUCAAUGUGACAGUGGGU
AACUAA

### 26. NGO1834: NC_002946.2:c1804153-1803875

DNA (- strand): SEQ ID NO: 103

ATGGCTCGTTCATTGAAAAAAGGCCCATATGTAGACCTGCATTTGCTGAAAAAAGTA
GATGCTGTTCGCGCAAGCAACGACAAACGCCCGATTAAAACCTGGTCTCGTCGTTCT
ACCATTCTGCCTGATTTTATCGGTCTGACCATTGCCGTGCACAACGGTCGCACCCAT
GTGCCTGTGTTTATCAGCGACAATATGGTTGGTCATAAATTAGGCGAATTCTCATTG
ACCCGTACCTTTAAAGGCCACCTGGCCGATAAAAAGGCTAAAAAGAAATAA

cDNA: SEQ ID NO: 104

TTATTTCTTTTTAGCCTTTTTATCGGCCAGGTGGCCTTTAAAGGTACGGGTCAATGAG
AATTCGCCTAATTTATGACCAACCATATTGTCGCTGATAAACACAGGCACATGGGTG
CGACCGTTGTGCACGGCAATGGTCAGACCGATAAAATCAGGCAGAATGGTAGAACG
ACGAGACCAGGTTTTAATCGGGCGTTTGTCGTTGCTTGCGCGAACAGCATCTACTTT
TTTCAGCAAATGCAGGTCTACATATGGGCCTTTTTTCAATGAACGAGCCAT

RNA: SEQ ID NO: 105

AUGGCUCGUUCAUUGAAAAAAGGCCCAUAUGUAGACCUGCAUUUGCUGAAAAAA
GUAGAUGCUGUUCGCGCAAGCAACGACAAACGCCCGAUUAAAACCUGGUCUCGUC
GUUCUACCAUUCUGCCUGAUUUUAUCGGUCUGACCAUUGCCGUGCACAACGGUCG
CACCCAUGUGCCUGUGUUUAUCAGCGACAAUAUGGUUGGUCAUAAAUUAGGCGA
AUUCUCAUUGACCCGUACCUUUAAAGGCCACCUGGCCGAUAAAAAGGCUAAAAAG
AAAUAA

### 27. NGO1835: NC_002946.2:c1804992-1804159

DNA (- strand): SEQ ID NO: 106

ATGGCAATCGTTAAAATGAAGCCGACCTCTGCAGGCCGTCGCGGCATGGTTCGCGTG
GTAACAGAAGGTTTGCACAAAGGTGCACCTTATGCACCCTTGCTTGAAAAGAAAAA
TTCTACTGCCGGCCGTAACAATAATGGTCATATCACCACCCGTCACAAAGGCGGCGG
TCATAAACACCATTACCGTGTTGTAGACTTTAAACGTAACAAAGACGGCATTTCTGC
TAAAGTAGAGCGTATCGAATACGATCCTAACCGTACTGCCTTCATCGCACTGTTGTG
CTATGCAGACGGCGAGCGTCGTTACATCATCGCTCCTCGCGGTATTCAAGCCGGTGT
CGTATTGGTTTCCGGTGCTGAAGCTGCCATCAAAGTAGGCAACACCCTGCCGATCCG
CAACATTCCCGTTGGTACGACTATCCACTGTATCGAAATGAAACCCGGTAAAGGTGC
TCAAATCGCACGTTCTGCCGGTGCTTCTGCGGTATTGTTGGCTAAAGAAGGTGCATA
CGCTCAAGTCCGTCTGCGCTCTGGCGAAGTTCGTAAAATCAACGTAGATTGCCGTGC
GACCATCGGTGAAGTCGGTAACGAAGAGCAAAGCCTGAAAAAAATCGGTAAAGCC
GGTGCTAACCGTTGGCGCGGTATTCGTCCGACCGTACGCGGTGTTGTCATGAATCCC
GTCGATCACCCGCATGGTGGTGGTGAAGGCCGTACCGGCGAAGCCCGCGAACCGGT
TAGTCCATGGGGTACTCCTGCTAAAGGCTACCGCACTCGTAATAACAAACGCACGG
ATAATATGATTGTTCGTCGCCGTTACTCAAATAAAGGTTAA

cDNA: SEQ ID NO: 107

TTAACCTTTATTTGAGTAACGGCGACGAACAATCATATTATCCGTGCGTTTGTTATTA
CGAGTGCGGTAGCCTTTAGCAGGAGTACCCCATGGACTAACCGGTTCGCGGGCTTCG
CCGGTACGGCCTTCACCACCACCATGCGGGTGATCGACGGGATTCATGACAACACC
GCGTACGGTCGGACGAATACCGCGCCAACGGTTAGCACCGGCTTTACCGATTTTTTT
CAGGCTTTGCTCTTCGTTACCGACTTCACCGATGGTCGCACGGCAATCTACGTTGATT
TTACGAACTTCGCCAGAGCGCAGACGGACTTGAGCGTATGCACCTTCTTTAGCCAAC
AATACCGCAGAAGCACCGGCAGAACGTGCGATTTGAGCACCTTTACCGGGTTTCATT
TCGATACAGTGGATAGTCGTACCAACGGGAATGTTGCGGATCGGCAGGGTGTTGCCT
ACTTTGATGGCAGCTTCAGCACCGGAAACCAATACGACACCGGCTTGAATACCGCG
AGGAGCGATGATGTAACGACGCTCGCCGTCTGCATAGCACAACAGTGCGATGAAGG
CAGTACGGTTAGGATCGTATTCGATACGCTCTACTTTAGCAGAAATGCCGTCTTTGTT
ACGTTTAAAGTCTACAACACGGTAATGGTGTTTATGACCGCCGCCTTTGTGACGGGT
GGTGATATGACCATTATTGTTACGGCCGGCAGTAGAATTTTTCTTTTCAAGCAAGGG
TGCATAAGGTGCACCTTTGTGCAAACCTTCTGTTACCACGCGAACCATGCCGCGACG
GCCTGCAGAGGTCGGCTTCATTTTAACGATTGCCAT

RNA: SEQ ID NO: 108

AUGGCAAUCGUUAAAAUGAAGCCGACCUCUGCAGGCCGUCGCGGCAUGGUUCGCG
UGGUAACAGAAGGUUUGCACAAAGGUGCACCUUAUGCACCCUUGCUUGAAAAGA
AAAAUUCUACUGCCGGCCGUAACAAUAAUGGUCAUAUCACCACCCGUCACAAAGG
CGGCGGUCAUAAACACCAUUACCGUGUUGUAGACUUUAAACGUAACAAAGACGG
CAUUUCUGCUAAAGUAGAGCGUAUCGAAUACGAUCCUAACCGUACUGCCUUCAUC
GCACUGUUGUGCUAUGCAGACGGCGAGCGUCGUUACAUCAUCGCUCCUCGCGGUA
UUCAAGCCGGUGUCGUAUUGGUUUCCGGUGCUGAAGCUGCCAUCAAAGUAGGCA
ACACCCUGCCGAUCCGCAACAUUCCCGUUGGUACGACUAUCCACUGUAUCGAAAU
GAAACCCGGUAAAGGUGCUCAAAUCGCACGUUCUGCCGGUGCUUCUGCGGUAUUG
UUGGCUAAAGAAGGUGCAUACGCUCAAGUCCGUCUGCGCUCUGGCGAAGUUCGU
AAAAUCAACGUAGAUUGCCGUGCGACCAUCGGUGAAGUCGGUAACGAAGAGCAA
AGCCUGAAAAAAAUCGGUAAAGCCGGUGCUAACCGUUGGCGCGGUAUUCGUCCG
ACCGUACGCGGUGUUGUCAUGAAUCCCGUCGAUCACCCGCAUGGUGGUGGUGAAG
GCCGUACCGGCGAAGCCCGCGAACCGGUUAGUCCAUGGGGUACUCCUGCUAAAGG
CUACCGCACUCGUAAUAACAAACGCACGGAUAAUAUGAUUGUUCGUCGCCGUUAC
UCAAAUAAAGGUUAA

### 28. NGO1837: NC_002946.2:c1805929-1805309

DNA (- strand): SEQ ID NO: 109

ATGGAATTGAAAGTAATTGACGCTAAAGGACAAGTTTCAGGCAGCCTGTCTGTTTCT
GATGCTTTGTTCGCCCGCGAATACAATGAAGCGTTGGTTCACCAGCTGGTAAATGCC
TACTTGGCAAACGCCCGCTCTGGTAACCGTGCTCAAAAAACCCGTGCCGAAGTAAA
ACACTCAACCAAAAAACCATGGCGTCAAAAAGGTACCGGCCGCGCCCGTTCCGGTA
TGACTTCTTCTCCGCTGTGGCGTAAAGGCGGTCGCGCGTTCCCGAACAAACCCGACG
AAAACTTCACTCAAAAAGTAAACCGTAAAATGTACCGTGCCGGTATGGCGACTATC
CTGTCCCAATTGGCGCGTGACGAGCGTTTGTTTGTGATTGAGGCGTTGACTGCCGAA
ACTCCCAAAACCAAAGTTTTTGCCGAACAAGTAAAAAATTTGGCTCTGGAGCAAGT
GCTGTTTGTAACCAAACGGCTCGACGAGAATGTTTACTTGGCTTCACGCAACTTGCC
AAACGTATTGGTTTTGGAAGCTCAACAAGTTGATCCTTACAGCTTGCTGCGTTATAA
AAAAGTAATCATCACTAAAGATGCGGTTGCACAATTAGAGGAGCAATGGGTATGA

cDNA: SEQ ID NO: 110

TCATACCCATTGCTCCTCTAATTGTGCAACCGCATCTTTAGTGATGATTACTTTTTTA
TAACGCAGCAAGCTGTAAGGATCAACTTGTTGAGCTTCCAAAACCAATACGTTTGGC
AAGTTGCGTGAAGCCAAGTAAACATTCTCGTCGAGCCGTTTGGTTACAAACAGCACT
TGCTCCAGAGCCAAATTTTTTACTTGTTCGGCAAAAACTTTGGTTTTGGGAGTTTCGG
CAGTCAACGCCTCAATCACAAACAAACGCTCGTCACGCGCCAATTGGGACAGGATA
GTCGCCATACCGGCACGGTACATTTTACGGTTTACTTTTTGAGTGAAGTTTTCGTCGG
GTTTGTTCGGGAACGCGCGACCGCCTTTACGCCACAGCGGAGAAGAAGTCATACCG
GAACGGGCGCGGCCGGTACCTTTTTGACGCCATGGTTTTTTGGTTGAGTGTTTTACTT
CGGCACGGGTTTTTTGAGCACGGTTACCAGAGCGGGCGTTTGCCAAGTAGGCATTTA
CCAGCTGGTGAACCAACGCTTCATTGTATTCGCGGGCGAACAAAGCATCAGAAACA
GACAGGCTGCCTGAAACTTGTCCTTTAGCGTCAATTACTTTCAATTCCAT

RNA: SEQ ID NO: 111

AUGGAAUUGAAAGUAAUUGACGCUAAAGGACAAGUUUCAGGCAGCCUGUCUGUU
UCUGAUGCUUUGUUCGCCCGCGAAUACAAUGAAGCGUUGGUUCACCAGCUGGUA
AAUGCCUACUUGGCAAACGCCCGCUCUGGUAACCGUGCUCAAAAAACCCGUGCCG
AAGUAAAACACUCAACCAAAAAACCAUGGCGUCAAAAAGGUACCGGCCGCGCCCG
UUCCGGUAUGACUUCUUCUCCGCUGUGGCGUAAAGGCGGUCGCGCGUUCCCGAAC
AAACCCGACGAAAACUUCACUCAAAAAGUAAACCGUAAAAUGUACCGUGCCGGUA
UGGCGACUAUCCUGUCCCAAUUGGCGCGUGACGAGCGUUUGUUUGUGAUUGAGG
CGUUGACUGCCGAAACUCCCAAAACCAAAGUUUUUGCCGAACAAGUAAAAAAUU
UGGCUCUGGAGCAAGUGCUGUUUGUAACCAAACGGCUCGACGAGAAUGUUUACU
UGGCUUCACGCAACUUGCCAAACGUAUUGGUUUUGGAAGCUCAACAAGUUGAUC
CUUACAGCUUGCUGCGUUAUAAAAAAGUAAUCAUCACUAAAGAUGCGGUUGCAC
AAUUAGAGGAGCAAUGGGUAUGA

### 29. NGO1843: NC_002946.2:c1811065-1808960

DNA (- strand): SEQ ID NO: 112

ATGGCTCGTAAGACCCCGATCAGCCTGTACCGCAACATCGGTATTTCCGCCCATATC
GATGCGGGTAAAACCACGACGACAGAACGTATTTTGTTCTATACCGGTTTGACCCAC
AAGCTGGGCGAAGTGCATGACGGTGCGGCTACTACCGACTACATGGAACAAGAGCA
AGAGCGCGGTATTACCATTACCTCCGCTGCCGTTACTTCCTACTGGTCCGGTATGGC
GAAACAATTCCCCGAGCACCGCTTCAACATCATCGACACCCCGGGGCACGTTGACTT
TACCGTAGAGGTAGAGCGTTCTATGCGTGTATTGGACGGCGCGGTAATGGTTTACTG
TGCGGTGGGCGGTGTTCAACCGCAATCTGAAACCGTATGGCGGCAAGCCAACAAAT
ACCAAGTTCCGCGCTTGGCGTTTGTCAATAAAATGGACCGCCAAGGTGCCAACTTCT
TCCGCGTTGTCGAGCAAATGAAAACCCGTTTGCGCGCAAACCCCGTACCTATCGTCA
TTCCGGTAGGCGCGGAAGACAGTTTTACCGGTGTTGTCGATTTGCTGAAAATGAAAT
CTATCATCTGGAATGAAGCCGATAAAGGTACAACCTTTACCTATGGCGATATTCCTG
CCGAATTGGTCGAAACTGCCGAAGAATGGCGTCAAAATATGATTGAAGCCGCAGCC
GAAGCCAGCGAAGAACTGATGGACAAATACTTGGGCGGTGAAGATCTGGCCGAAGA
AGAAATCGTAGGCGCGTTGCGTCAACGTACTTTGGCAGGCGAAATTCAGCCTATGCT
GTGCGGTTCTGCATTTAAAAACAAAGGTGTTCAACGTATGTTGGACGCAGTTGTAGA
ATTGCTGCCAGCTCCTACCGATATTCCTCCGGTTCAAGGTGTTAATCCTAACACTGA
AGAAGCCGACAGCCGTCAAGCCAGCGATGAAGAGAAATTCTCTGCATTGGCATTCA
AAATGTTGAACGACAAATACGTCGGTCAGCTGACCTTTATCCGCGTTTACTCAGGCG
TAGTAAAATCCGGCGATACCGTACTGAATTCTGTAAAAGGCACTCGCGAACGTATCG
GTCGTTTGGTGCAAATGACTGCCGCAGACCGTACTGAAATCGAAGAAGTACGCGCT
GGCGACATCGCAGCCGCTATCGGTCTGAAAGACGTTACTACCGGTGAAACCTTGTGT
GCGGAAAGCGCGCCGATTATCTTGGAACGTATGGAATTCCCCGAGCCGGTAATCCAT
ATTGCCGTTGAGCCGAAAACCAAAGCCGACCAAGAGAAAATGGGTATCGCCCTGAA
CCGCTTGGCTAAAGAAGACCCTTCTTTCCGCGTTCGTACAGACGAAGAATCCGGTCA
AACCATTATTTCCGGTATGGGTGAGCTGCACTTGGAAATTATTGTTGACCGTATGAA
ACGCGAATTCGGTGTGGAAGCAAATATCGGTGCACCTCAAGTGGCTTACCGTGAAA
CTATCCGCAAAGCCGTTAAAGCTGAATACAAACATGCAAAACAATCCGGTGGTAAA
GGTCAATACGGTCACGTTGTGATTGAAATGGAACCTATGGAACCGGGTGGTGAAGG
TTACGAGTTTATCGATGAAATTAAAGGTGGTGTGATTCCTCGCGAATTTATTCCGTCT
GTCGATAAAGGTATCCGCGATACGTTGCCTAACGGTATCGTTGCCGGCTATCCTGTA
GTTGACGTACGTATCCGTCTGGTATTCGGTTCTTACCATGATGTCGACTCTTCCCAAT
TGGCATTTGAATTGGCTGCTTCTCAAGCGTTTAAAGAAGGTATGCGTCAAGCATCTC
CTGCCCTGCTTGAGCCGATTATGGCAGTTGAAGTGGAAACTCCGGAAGAATACATG
GGCGACGTAATGGGCGACTTGAACCGCCGTCGCGGTGTTGTATTGGGTATGGATGAT
GACGGTATCGGCGGTAAAAAAGTCCGTGCCGAAGTACCTCTGGCAGAAATGTTCGG
TTACTCGACCGACCTGCGTTCTGCAACCCAAGGCCGCGCTACTTACTCTATGGAGTT
CAAGAAATATTCTGAAGCTCCTGCCCACATAGCTGCTGCTGTAACTGAAGCCCGTAA
AGGCTAA

cDNA: SEQ ID NO: 113

TTAGCCTTTACGGGCTTCAGTTACAGCAGCAGCTATGTGGGCAGGAGCTTCAGAATA
TTTCTTGAACTCCATAGAGTAAGTAGCGCGGCCTTGGGTTGCAGAACGCAGGTCGGT
CGAGTAACCGAACATTTCTGCCAGAGGTACTTCGGCACGGACTTTTTTACCGCCGAT
ACCGTCATCATCCATACCCAATACAACACCGCGACGGCGGTTCAAGTCGCCCATTAC
GTCGCCCATGTATTCTTCCGGAGTTTCCACTTCAACTGCCATAATCGGCTCAAGCAG
GGCAGGAGATGCTTGACGCATACCTTCTTTAAACGCTTGAGAAGCAGCCAATTCAAA
TGCCAATTGGGAAGAGTCGACATCATGGTAAGAACCGAATACCAGACGGATACGTA
CGTCAACTACAGGATAGCCGGCAACGATACCGTTAGGCAACGTATCGCGGATACCT
TTATCGACAGACGGAATAAATTCGCGAGGAATCACACCACCTTTAATTTCATCGATA
AACTCGTAACCTTCACCACCCGGTTCCATAGGTTCCATTTCAATCACAACGTGACCG
TATTGACCTTTACCACCGGATTGTTTTGCATGTTTGTATTCAGCTTTAACGGCTTTGC
GGATAGTTTCACGGTAAGCCACTTGAGGTGCACCGATATTTGCTTCCACACCGAATT
CGCGTTTCATACGGTCAACAATAATTTCCAAGTGCAGCTCACCCATACCGGAAATAA
TGGTTTGACCGGATTCTTCGTCTGTACGAACGCGGAAAGAAGGGTCTTCTTTAGCCA
AGCGGTTCAGGGCGATACCCATTTTCTCTTGGTCGGCTTTGGTTTTCGGCTCAACGGC
AATATGGATTACCGGCTCGGGGAATTCCATACGTTCCAAGATAATCGGCGCGCTTTC
CGCACACAAGGTTTCACCGGTAGTAACGTCTTTCAGACCGATAGCGGCTGCGATGTC
GCCAGCGCGTACTTCTTCGATTTCAGTACGGTCTGCGGCAGTCATTTGCACCAAACG
ACCGATACGTTCGCGAGTGCCTTTTACAGAATTCAGTACGGTATCGCCGGATTTTAC
TACGCCTGAGTAAACGCGGATAAAGGTCAGCTGACCGACGTATTTGTCGTTCAACAT
TTTGAATGCCAATGCAGAGAATTTCTCTTCATCGCTGGCTTGACGGCTGTCGGCTTCT
TCAGTGTTAGGATTAACACCTTGAACCGGAGGAATATCGGTAGGAGCTGGCAGCAA
TTCTACAACTGCGTCCAACATACGTTGAACACCTTTGTTTTTAAATGCAGAACCGCA
CAGCATAGGCTGAATTTCGCCTGCCAAAGTACGTTGACGCAACGCGCCTACGATTTC
TTCTTCGGCCAGATCTTCACCGCCCAAGTATTTGTCCATCAGTTCTTCGCTGGCTTCG
GCTGCGGCTTCAATCATATTTTGACGCCATTCTTCGGCAGTTTCGACCAATTCGGCAG
GAATATCGCCATAGGTAAAGGTTGTACCTTTATCGGCTTCATTCCAGATGATAGATT
TCATTTTCAGCAAATCGACAACACCGGTAAAACTGTCTTCCGCGCCTACCGGAATGA
CGATAGGTACGGGGTTTGCGCGCAAACGGGTTTTCATTTGCTCGACAACGCGGAAG
AAGTTGGCACCTTGGCGGTCCATTTTATTGACAAACGCCAAGCGCGGAACTTGGTAT
TTGTTGGCTTGCCGCCATACGGTTTCAGATTGCGGTTGAACACCGCCCACCGCACAG
TAAACCATTACCGCGCCGTCCAATACACGCATAGAACGCTCTACCTCTACGGTAAAG
TCAACGTGCCCCGGGGTGTCGATGATGTTGAAGCGGTGCTCGGGGAATTGTTTCGCC
ATACCGGACCAGTAGGAAGTAACGGCAGCGGAGGTAATGGTAATACCGCGCTCTTG
CTCTTGTTCCATGTAGTCGGTAGTAGCCGCACCGTCATGCACTTCGCCCAGCTTGTGG
GTCAAACCGGTATAGAACAAAATACGTTCTGTCGTCGTGGTTTTACCCGCATCGATA
TGGGCGGAAATACCGATGTTGCGGTACAGGCTGATCGGGGTCTTACGAGCCAT

RNA: SEQ ID NO: 114

AUGGCUCGUAAGACCCCGAUCAGCCUGUACCGCAACAUCGGUAUUUCCGCCCAUA
UCGAUGCGGGUAAAACCACGACGACAGAACGUAUUUUGUUCUAUACCGGUUUGA
CCCACAAGCUGGGCGAAGUGCAUGACGGUGCGGCUACUACCGACUACAUGGAACA
AGAGCAAGAGCGCGGUAUUACCAUUACCUCCGCUGCCGUUACUUCCUACUGGUCC
GGUAUGGCGAAACAAUUCCCCGAGCACCGCUUCAACAUCAUCGACACCCCGGGGC
ACGUUGACUUUACCGUAGAGGUAGAGCGUUCUAUGCGUGUAUUGGACGGCGCGG
UAAUGGUUUACUGUGCGGUGGGCGGUGUUCAACCGCAAUCUGAAACCGUAUGGC
GGCAAGCCAACAAAUACCAAGUUCCGCGCUUGGCGUUUGUCAAUAAAAUGGACCG
CCAAGGUGCCAACUUCUUCCGCGUUGUCGAGCAAAUGAAAACCCGUUUGCGCGCA
AACCCCGUACCUAUCGUCAUUCCGGUAGGCGCGGAAGACAGUUUUACCGGUGUUG
UCGAUUUGCUGAAAAUGAAAUCUAUCAUCUGGAAUGAAGCCGAUAAAGGUACAA
CCUUUACCUAUGGCGAUAUUCCUGCCGAAUUGGUCGAAACUGCCGAAGAAUGGCG
UCAAAAUAUGAUUGAAGCCGCAGCCGAAGCCAGCGAAGAACUGAUGGACAAAUA
CUUGGGCGGUGAAGAUCUGGCCGAAGAAGAAAUCGUAGGCGCGUUGCGUCAACG
UACUUUGGCAGGCGAAAUUCAGCCUAUGCUGUGCGGUUCUGCAUUUAAAAACAA
AGGUGUUCAACGUAUGUUGGACGCAGUUGUAGAAUUGCUGCCAGCUCCUACCGA
UAUUCCUCCGGUUCAAGGUGUUAAUCCUAACACUGAAGAAGCCGACAGCCGUCAA
GCCAGCGAUGAAGAGAAAUUCUCUGCAUUGGCAUUCAAAAUGUUGAACGACAAA
UACGUCGGUCAGCUGACCUUUAUCCGCGUUUACUCAGGCGUAGUAAAAUCCGGCG
AUACCGUACUGAAUUCUGUAAAAGGCACUCGCGAACGUAUCGGUCGUUUGGUGC
AAAUGACUGCCGCAGACCGUACUGAAAUCGAAGAAGUACGCGCUGGCGACAUCGC
AGCCGCUAUCGGUCUGAAAGACGUUACUACCGGUGAAACCUUGUGUGCGGAAAG
CGCGCCGAUUAUCUUGGAACGUAUGGAAUUCCCCGAGCCGGUAAUCCAUAUUGCC
GUUGAGCCGAAAACCAAAGCCGACCAAGAGAAAAUGGGUAUCGCCCUGAACCGCU
UGGCUAAAGAAGACCCUUCUUUCCGCGUUCGUACAGACGAAGAAUCCGGUCAAAC
CAUUAUUUCCGGUAUGGGUGAGCUGCACUUGGAAAUUAUUGUUGACCGUAUGAA
ACGCGAAUUCGGUGUGGAAGCAAAUAUCGGUGCACCUCAAGUGGCUUACCGUGA
AACUAUCCGCAAAGCCGUUAAAGCUGAAUACAAACAUGCAAAACAAUCCGGUGG
UAAAGGUCAAUACGGUCACGUUGUGAUUGAAAUGGAACCUAUGGAACCGGGUGG
UGAAGGUUACGAGUUUAUCGAUGAAAUUAAAGGUGGUGUGAUUCCUCGCGAAUU
UAUUCCGUCUGUCGAUAAAGGUAUCCGCGAUACGUUGCCUAACGGUAUCGUUGCC
GGCUAUCCUGUAGUUGACGUACGUAUCCGUCUGGUAUUCGGUUCUUACCAUGAU
GUCGACUCUUCCCAAUUGGCAUUUGAAUUGGCUGCUUCUCAAGCGUUUAAAGAA
GGUAUGCGUCAAGCAUCUCCUGCCCUGCUUGAGCCGAUUAUGGCAGUUGAAGUG
GAAACUCCGGAAGAAUACAUGGGCGACGUAAUGGGCGACUUGAACCGCCGUCGCG
GUGUUGUAUUGGGUAUGGAUGAUGACGGUAUCGGCGGUAAAAAAGUCCGUGCCG
AAGUACCUCUGGCAGAAAUGUUCGGUUACUCGACCGACCUGCGUUCUGCAACCCA
AGGCCGCGCUACUUACUCUAUGGAGUUCAAGAAAUAUUCUGAAGCUCCUGCCCAC
AUAGCUGCUGCUGUAACUGAAGCCCGUAAAGGCUAA

### 30. NGO1844: NC_002946.2:c1811554-1811084

DNA (- strand): SEQ ID NO: 115

ATGCCAAGACGTAGAGAAGTCCCCAAGCGCGACGTACTGCCAGATCCTAAATTCGG
TAGCGTCGAGTTGACCAAATTCATGAACGTATTGATGATTGACGGTAAAAAATCCGT
TGCCGAGCGTATCGTTTACGGTGCGTTGGAACAGATTGAGAAAAAAACCGGCAAAG
CAGCAATCGAAGTATTTAACGAAGCCATTGCAAACTCCAAACCTATCGTGGAAGTG
AAAAGCCGCCGTGTAGGTGGTGCAAACTACCAAGTTCCTGTTGAAGTTCGTCCTTCA
CGCCGTCTGGCTTTGGCAATGCGTTGGGTTCGCGACGCGGCCCGCAAACGTGGTGAG
AAATCCATGGATCTGCGTTTGGCAGGCGAGTTGATTGATGCGTCCGAAGGCCGTGGC
GGTGCGTTGAAAAAACGTGAAGAAGTACACCGTATGGCTGAAGCCAACAAAGCATT
CTCTCACTTCCGTTTCTAA

cDNA: SEQ ID NO: 116

TTAGAAACGGAAGTGAGAGAATGCTTTGTTGGCTTCAGCCATACGGTGTACTTCTTC
ACGTTTTTTCAACGCACCGCCACGGCCTTCGGACGCATCAATCAACTCGCCTGCCAA
ACGCAGATCCATGGATTTCTCACCACGTTTGCGGGCCGCGTCGCGAACCCAACGCAT
TGCCAAAGCCAGACGGCGTGAAGGACGAACTTCAACAGGAACTTGGTAGTTTGCAC
CACCTACACGGCGGCTTTTCACTTCCACGATAGGTTTGGAGTTTGCAATGGCTTCGTT
AAATACTTCGATTGCTGCTTTGCCGGTTTTTTTCTCAATCTGTTCCAACGCACCGTAA
ACGATACGCTCGGCAACGGATTTTTTACCGTCAATCATCAATACGTTCATGAATTTG
GTCAACTCGACGCTACCGAATTTAGGATCTGGCAGTACGTCGCGCTTGGGGACTTCT
CTACGTCTTGGCAT

RNA: SEQ ID NO: 117

AUGCCAAGACGUAGAGAAGUCCCCAAGCGCGACGUACUGCCAGAUCCUAAAUUCG
GUAGCGUCGAGUUGACCAAAUUCAUGAACGUAUUGAUGAUUGACGGUAAAAAAU
CCGUUGCCGAGCGUAUCGUUUACGGUGCGUUGGAACAGAUUGAGAAAAAAACCG
GCAAAGCAGCAAUCGAAGUAUUUAACGAAGCCAUUGCAAACUCCAAACCUAUCGU
GGAAGUGAAAAGCCGCCGUGUAGGUGGUGCAAACUACCAAGUUCCUGUUGAAGU
UCGUCCUUCACGCCGUCUGGCUUUGGCAAUGCGUUGGGUUCGCGACGCGGCCCGC
AAACGUGGUGAGAAAUCCAUGGAUCUGCGUUUGGCAGGCGAGUUGAUUGAUGCG
UCCGAAGGCCGUGGCGGUGCGUUGAAAAAACGUGAAGAAGUACACCGUAUGGCU
GAAGCCAACAAAGCAUUCUCUCACUUCCGUUUCUAA

### 31. NGO1845: NC_002946.2:c1812043-1811672

DNA (- strand): SEQ ID NO: 118

ATGCCAACTATCAACCAATTGGTACGCAAAGGCCGTCAAAAGCCCGTGTACGTAAA
CAAAGTGCCCGCACTGGAAGCCTGCCCGCAAAAACGCGGCGTGTGCACCCGTGTAT
ACACGACTACCCCTAGAAAACCTAACTCTGCATTGCGTAAAGTATGTAAAGTCCGCC
TGACCAACGGTTTTGAAGTCATTTCATATATCGGCGGTGAAGGCCACAACCTGCAAG
AGCACAGCGTCGTACTGATTCGCGGCGGCCGTGTAAAAGACTTGCCGGGTGTACGTT
ACCACACTGTACGCGGTTCTTTGGATACTGCAGGTGTTAAAGACCGCAAACAAGCCC
GTTCTAAATACGGTGCTAAGCGTCCTAAATAA

cDNA: SEQ ID NO: 119

TTATTTAGGACGCTTAGCACCGTATTTAGAACGGGCTTGTTTGCGGTCTTTAACACCT
GCAGTATCCAAAGAACCGCGTACAGTGTGGTAACGTACACCCGGCAAGTCTTTTACA
CGGCCGCCGCGAATCAGTACGACGCTGTGCTCTTGCAGGTTGTGGCCTTCACCGCCG
ATATATGAAATGACTTCAAAACCGTTGGTCAGGCGGACTTTACATACTTTACGCAAT
GCAGAGTTAGGTTTTCTAGGGGTAGTCGTGTATACACGGGTGCACACGCCGCGTTTT
TGCGGGCAGGCTTCCAGTGCGGGCACTTTGTTTACGTACACGGGCTTTTGACGGCCT
TTGCGTACCAATTGGTTGATAGTTGGCAT

RNA: SEQ ID NO: 120

AUGCCAACUAUCAACCAAUUGGUACGCAAAGGCCGUCAAAAGCCCGUGUACGUAA
ACAAAGUGCCCGCACUGGAAGCCUGCCCGCAAAAACGCGGCGUGUGCACCCGUGU
AUACACGACUACCCCUAGAAAACCUAACUCUGCAUUGCGUAAAGUAUGUAAAGUC
CGCCUGACCAACGGUUUUGAAGUCAUUUCAUAUAUCGGCGGUGAAGGCCACAACC
UGCAAGAGCACAGCGUCGUACUGAUUCGCGGCGGCCGUGUAAAAGACUUGCCGGG
UGUACGUUACCACACUGUACGCGGUUCUUUGGAUACUGCAGGUGUUAAAGACCG
CAAACAAGCCCGUUCUAAAUACGGUGCUAAGCGUCCUAAAUAA

### 32. NGO1890: NC_002946.2:c1857972-1856758

DNA (- strand): SEQ ID NO: 121

ATGGAATGGGCGTTTAACAGTTATTACACCTTGATTGCCGCCACTTTGGTTTTGTTGG
TCGGCAAGGTTTTGGTTAAGAAAATCAAAATCTTGCGTGATTTTAACATCCCCGAAC
CCGTGGCGGGCGGGCTGATTGCCGCGATTATCCTGTTTGCGCTGCACGAGGCGTACG
GCGTGAGCTTCAAATTTGAGAAACCGCTGCAAAATGCGTTTATGCTGATTTTCTTCA
CGTCCATCGGCTTGAGCGCGGATTTTTCCCGTTTGAAGGCGGGCGGTTTGCCGCTGG
TGGTTTTTACCGCGATTGTGGGCGGATTTATCTTGGTGCAAAACTTTGTCGGGGTCG
GACTGGCTACGGCTTTGGGTTTGGACCCGCTCATCGGTCTGATTACCGGTTCGGTGT
CGCTGACGGGCGGACACGGCACGTCAGGTGCGTGGGGACCTAATTTTGAAACGCAA
TACGGCTTGGTCGGCGCAACCGGTTTGGGTATTGCTTCGGTTACTTTCGGGCTGGTGT
TCGGCGGCCTGATCGGAGGGCCGGTTGCGCGCCGCCTGATCAACAAAATGGGCCGC
AAACCGGTTGAAAACACAAAACAGGATCAGGACGACAACGCGGACGACGTGTTCG
AGCAGGCAAAACGCACCCGCCTGATTACGGCGGAATCTGCCGTTGAAACGCTTGCC
ATGTTTGCCGCGTGTCTGGCGTTTGCCGAGATTATGGACGGTTTCGACAAAGAATAC
CTGTTCGACCTGCCCAAATTCGTGTGGTGTCTGTTTGGCGGCGTGGTTATCCGCAAC
ATCCTTACCGCCGCATTCAAGGTCAATATGTTCGACCGTGCCATCGATGTGTTCGGC
AATGCTTCGCTTTCGCTTTTCTTGGCAATGGCGTTGCTGAATTTGAAACTGTGGGAGC
TGACCGGTTTGGCGGGGTCTGTAACCGTGATTCTTGCAGTACAAACCGCAGTGATGG
TTTTGTACGCGACTTTTGTTACCTATGTCTTTATGGGGCGCGACTATGATGCCGCAGT
ATTGGCTGCCGGCCACTGCGGTTTCGGTTTGGGCGCAACGCCGACGGCGGTGGCAA
ATATGCAGTCCGTCACGCATACTTTCGGCGCGTCACATAAGGCGTTTTTGATTGTGC
CTATGGTCGGCGCGTTCTTTGTCGATTTGATTAATGCCGCGATTCTCACCGGTTTTGT
GAATTTCTTTAAAGGCTGA

cDNA: SEQ ID NO: 122

TCAGCCTTTAAAGAAATTCACAAAACCGGTGAGAATCGCGGCATTAATCAAATCGA
CAAAGAACGCGCCGACCATAGGCACAATCAAAAACGCCTTATGTGACGCGCCGAAA
GTATGCGTGACGGACTGCATATTTGCCACCGCCGTCGGCGTTGCGCCCAAACCGAAA
CCGCAGTGGCCGGCAGCCAATACTGCGGCATCATAGTCGCGCCCCATAAAGACATA
GGTAACAAAAGTCGCGTACAAAACCATCACTGCGGTTTGTACTGCAAGAATCACGG
TTACAGACCCCGCCAAACCGGTCAGCTCCCACAGTTTCAAATTCAGCAACGCCATTG
CCAAGAAAAGCGAAAGCGAAGCATTGCCGAACACATCGATGGCACGGTCGAACAT
ATTGACCTTGAATGCGGCGGTAAGGATGTTGCGGATAACCACGCCGCCAAACAGAC
ACCACACGAATTTGGGCAGGTCGAACAGGTATTCTTTGTCGAAACCGTCCATAATCT
CGGCAAACGCCAGACACGCGGCAAACATGGCAAGCGTTTCAACGGCAGATTCCGCC
GTAATCAGGCGGGTGCGTTTTGCCTGCTCGAACACGTCGTCCGCGTTGTCGTCCTGA
TCCTGTTTTGTGTTTTCAACCGGTTTGCGGCCCATTTTGTTGATCAGGCGGCGCGCAA
CCGGCCCTCCGATCAGGCCGCCGAACACCAGCCCGAAAGTAACCGAAGCAATACCC
AAACCGGTTGCGCCGACCAAGCCGTATTGCGTTTCAAAATTAGGTCCCCACGCACCT
GACGTGCCGTGTCCGCCCGTCAGCGACACCGAACCGGTAATCAGACCGATGAGCGG
GTCCAAACCCAAAGCCGTAGCCAGTCCGACCCCGACAAAGTTTTGCACCAAGATAA
ATCCGCCCACAATCGCGGTAAAAACCACCAGCGGCAAACCGCCCGCCTTCAAACGG
GAAAAATCCGCGCTCAAGCCGATGGACGTGAAGAAAATCAGCATAAACGCATTTTG
CAGCGGTTTCTCAAATTTGAAGCTCACGCCGTACGCCTCGTGCAGCGCAAACAGGAT
AATCGCGGCAATCAGCCCGCCCGCCACGGGTTCGGGGATGTTAAAATCACGCAAGA
TTTTGATTTTCTTAACCAAAACCTTGCCGACCAACAAAACCAAAGTGGCGGCAATCA
AGGTGTAATAACTGTTAAACGCCCATTCCAT

RNA: SEQ ID NO: 123

AUGGAAUGGGCGUUUAACAGUUAUUACACCUUGAUUGCCGCCACUUUGGUUUUG
UUGGUCGGCAAGGUUUUGGUUAAGAAAAUCAAAAUCUUGCGUGAUUUUAACAUC
CCCGAACCCGUGGCGGGCGGGCUGAUUGCCGCGAUUAUCCUGUUUGCGCUGCACG
AGGCGUACGGCGUGAGCUUCAAAUUUGAGAAACCGCUGCAAAAUGCGUUUAUGC
UGAUUUUCUUCACGUCCAUCGGCUUGAGCGCGGAUUUUUCCCGUUUGAAGGCGG
GCGGUUUGCCGCUGGUGGUUUUUACCGCGAUUGUGGGCGGAUUUAUCUUGGUGC
AAAACUUUGUCGGGGUCGGACUGGCUACGGCUUUGGGUUUGGACCCGCUCAUCG
GUCUGAUUACCGGUUCGGUGUCGCUGACGGGCGGACACGGCACGUCAGGUGCGUG
GGGACCUAAUUUUGAAACGCAAUACGGCUUGGUCGGCGCAACCGGUUUGGGUAU
UGCUUCGGUUACUUUCGGGCUGGUGUUCGGCGGCCUGAUCGGAGGGCCGGUUGC
GCGCCGCCUGAUCAACAAAAUGGGCCGCAAACCGGUUGAAAACACAAAACAGGAU
CAGGACGACAACGCGGACGACGUGUUCGAGCAGGCAAAACGCACCCGCCUGAUUA
CGGCGGAAUCUGCCGUUGAAACGCUUGCCAUGUUUGCCGCGUGUCUGGCGUUUGC
CGAGAUUAUGGACGGUUUCGACAAAGAAUACCUGUUCGACCUGCCCAAAUUCGU
GUGGUGUCUGUUUGGCGGCGUGGUUAUCCGCAACAUCCUUACCGCCGCAUUCAAG
GUCAAUAUGUUCGACCGUGCCAUCGAUGUGUUCGGCAAUGCUUCGCUUUCGCUUU
UCUUGGCAAUGGCGUUGCUGAAUUUGAAACUGUGGGAGCUGACCGGUUUGGCGG
GGUCUGUAACCGUGAUUCUUGCAGUACAAACCGCAGUGAUGGUUUUGUACGCGA
CUUUUGUUACCUAUGUCUUUAUGGGGCGCGACUAUGAUGCCGCAGUAUUGGCUG
CCGGCCACUGCGGUUUCGGUUUGGGCGCAACGCCGACGGCGGUGGCAAAUAUGCA
GUCCGUCACGCAUACUUUCGGCGCGUCACAUAAGGCGUUUUUGAUUGUGCCUAUG
GUCGGCGCGUUCUUUGUCGAUUUGAUUAAUGCCGCGAUUCUCACCGGUUUUGUG
AAUUUCUUUAAAGGCUGA

### 33. Ngo2024: Nc_002946.2:1995172-1995603

DNA (+ strand): SEQ ID NO: 124

ATGAAAACCTTTTCAGCGAAACCCCACGAGGTGAAGCGCGAATGGTTCGTCATCGA
TGCCCAAGACAAAGTCTTGGGTCGCGTTGCAACCGAAGTCGCCAGCCGTCTGCGTG
GCAAACACAAACCTGAATACACCCCCCACGTCGATACCGGCGATTACATCATCGTC
ATCAATGCGGACAAACTGCGTGTAACCGGTGCCAAATTCGAAGATAAAAAATACTT
CCGCCATTCCGGTTTTCCAGGCGGCATCTACGAGCGCACTTTCCGCGAAATGCAAGA
TCAATTCCCGGGCCGCGCTTTGGAGCAGGCTGTAAAAGGTATGCTGCCCAAAGGTCC
GCTGGGTTACGCCATGATTAAAAAACTGAAAGTGTACGCTGGTGCGGAGCATGCCC
ATGCTGCGCAACAACCCAAAGTTTTGGAACTGAAATAA

cDNA: SEQ ID NO: 125

TTATTTCAGTTCCAAAACTTTGGGTTGTTGCGCAGCATGGGCATGCTCCGCACCAGC
GTACACTTTCAGTTTTTTAATCATGGCGTAACCCAGCGGACCTTTGGGCAGCATACC
TTTTACAGCCTGCTCCAAAGCGCGGCCCGGGAATTGATCTTGCATTTCGCGGAAAGT
GCGCTCGTAGATGCCGCCTGGAAAACCGGAATGGCGGAAGTATTTTTTATCTTCGAA
TTTGGCACCGGTTACACGCAGTTTGTCCGCATTGATGACGATGATGTAATCGCCGGT
ATCGACGTGGGGGGTGTATTCAGGTTTGTGTTTGCCACGCAGACGGCTGGCGACTTC
GGTTGCAACGCGACCCAAGACTTTGTCTTGGGCATCGATGACGAACCATTCGCGCTT
CACCTCGTGGGGTTTCGCTGAAAAGGTTTTCAT

RNA: SEQ ID NO: 126

AUGAAAACCUUUUCAGCGAAACCCCACGAGGUGAAGCGCGAAUGGUUCGUCAUCG
AUGCCCAAGACAAAGUCUUGGGUCGCGUUGCAACCGAAGUCGCCAGCCGUCUGCG
UGGCAAACACAAACCUGAAUACACCCCCCACGUCGAUACCGGCGAUUACAUCAUC
GUCAUCAAUGCGGACAAACUGCGUGUAACCGGUGCCAAAUUCGAAGAUAAAAAA
UACUUCCGCCAUUCCGGUUUUCCAGGCGGCAUCUACGAGCGCACUUUCCGCGAAA
UGCAAGAUCAAUUCCCGGGCCGCGCUUUGGAGCAGGCUGUAAAAGGUAUGCUGCC
CAAAGGUCCGCUGGGUUACGCCAUGAUUAAAAAACUGAAAGUGUACGCUGGUGC
GGAGCAUGCCCAUGCUGCGCAACAACCCAAAGUUUUGGAACUGAAAUAA

### 34. NGO2098: NC_002946.2:c2078739-2077519

DNA (- strand): SEQ ID NO: 127

ATGACCCTGTTTTGCGAACAAGTCCCCTACCCCCGCCTTGCCGAAGAATTCGGCACG
CCGCTTTATGTGTACAGCCAATCCGCGCTGACCGGAGCATTTGAAAACTATCAAACC
GCCTTTGCCGCTTTGAACCCGCTTGTCTGCTACGCCGTCAAGGCAAACGGCAACCTG
AGCATTATCAAACACTTTGCTTCTTTGGGCAGCGGTTTTGACATTGTGTCGGGCGGC
GAATTGGCACGCGTTTTGGCGGCAGGCGGCGATGCGGCGAAAACGATTTTTTCCGGC
GTAGGCAAAAGCGAGGCGGAAATCGAGTTCGCGCTGAATGCCGGCGTAAAATGCTT
CAATATGGAAAGCATCCCCGAAATCGACCGCATTCAGAAAATTGCCGCGCGTTTGG
GCAAAACCGCGCCCGTCTCCCTGCGCGTCAATCCCGATGTCGATGCAAAAACCCATC
CCTACATCTCCACAGGTCTGAAAGCCAACAAATTCGGCATCGCCTACGCCGACGCGC
TCGAAGCCTACCGCCATGCCGCACAACAGCCCAATTTGAAAATCATCGGCATCGACT
GCCACATCGGTTCGCAACTGACCGACTTAAGCCCACTGGTCGAAGCCTGCGAACGC
ATTTTGATTTTGGTTGACGCTCTTGCCGCCGAAGGCATTGTTTTGGAACATTTGGACT
TAGGCGGCGGCGTCGGCATTGTTTACAAAGACGAAGGCGTCCCCGATTTGGGTGCGT
ATGCCCGAGCGGTTCAAAAACTGATGGGGACACGCCGTCTGAAACTCATTCTTGAGC
CAGGCCGCAGCTTGGTCGGCAACGCAGGTGCATTGCTGACGCGCGTCGAATTTGTCA
AACACGGTGAAGAGAAAAACTTTGTGATGGTCGATGCGGCGATGAACGATTTGATG
CGCCCAGCCCTATACGATGCCTACCACCACATCGAAGCGGTTGAAACCAAAAACAT
TGAGCCTCTGACCGCCAACATCGTCGGCCCGATTTGTGAAACCGGCGACTTCCTCGG
CAAAGACCGCACCATCGCCTGCGAAGAAGGCGATTTGCTGCTTATCCGCAGCGCGG
GCGCATACGGGGCCAGTATGGCTAGCAATTACAACACGCGCAACCGTGCGGCGGAG
GTGTTGGTTGACGGCGGCGGATACAAACTCATCCGCCGGCGCGAAACCTTGGAACA
GCAAATGGCAAACGAACTCGCCTGCCTATAA

cDNA: SEQ ID NO: 128

TTATAGGCAGGCGAGTTCGTTTGCCATTTGCTGTTCCAAGGTTTCGCGCCGGCGGAT
GAGTTTGTATCCGCCGCCGTCAACCAACACCTCCGCCGCACGGTTGCGCGTGTTGTA
ATTGCTAGCCATACTGGCCCCGTATGCGCCCGCGCTGCGGATAAGCAGCAAATCGCC
TTCTTCGCAGGCGATGGTGCGGTCTTTGCCGAGGAAGTCGCCGGTTTCACAAATCGG
GCCGACGATGTTGGCGGTCAGAGGCTCAATGTTTTTGGTTTCAACCGCTTCGATGTG
GTGGTAGGCATCGTATAGGGCTGGGCGCATCAAATCGTTCATCGCCGCATCGACCAT
CACAAAGTTTTTCTCTTCACCGTGTTTGACAAATTCGACGCGCGTCAGCAATGCACC
TGCGTTGCCGACCAAGCTGCGGCCTGGCTCAAGAATGAGTTTCAGACGGCGTGTCCC
CATCAGTTTTTGAACCGCTCGGGCATACGCACCCAAATCGGGGACGCCTTCGTCTTT
GTAAACAATGCCGACGCCGCCGCCTAAGTCCAAATGTTCCAAAACAATGCCTTCGG
CGGCAAGAGCGTCAACCAAAATCAAAATGCGTTCGCAGGCTTCGACCAGTGGGCTT
AAGTCGGTCAGTTGCGAACCGATGTGGCAGTCGATGCCGATGATTTTCAAATTGGGC
TGTTGTGCGGCATGGCGGTAGGCTTCGAGCGCGTCGGCGTAGGCGATGCCGAATTTG
TTGGCTTTCAGACCTGTGGAGATGTAGGGATGGGTTTTTGCATCGACATCGGGATTG
ACGCGCAGGGAGACGGGCGCGGTTTTGCCCAAACGCGCGGCAATTTTCTGAATGCG
GTCGATTTCGGGGATGCTTTCCATATTGAAGCATTTTACGCCGGCATTCAGCGCGAA
CTCGATTTCCGCCTCGCTTTTGCCTACGCCGGAAAAAATCGTTTTCGCCGCATCGCCG
CCTGCCGCCAAAACGCGTGCCAATTCGCCGCCCGACACAATGTCAAAACCGCTGCC
CAAAGAAGCAAAGTGTTTGATAATGCTCAGGTTGCCGTTTGCCTTGACGGCGTAGCA
GACAAGCGGGTTCAAAGCGGCAAAGGCGGTTTGATAGTTTTCAAATGCTCCGGTCA
GCGCGGATTGGCTGTACACATAAAGCGGCGTGCCGAATTCTTCGGCAAGGCGGGGG
TAGGGGACTTGTTCGCAAAACAGGGTCAT

RNA: SEQ ID NO: 129

AUGACCCUGUUUUGCGAACAAGUCCCCUACCCCCGCCUUGCCGAAGAAUUCGGCA
CGCCGCUUUAUGUGUACAGCCAAUCCGCGCUGACCGGAGCAUUUGAAAACUAUCA
AACCGCCUUUGCCGCUUUGAACCCGCUUGUCUGCUACGCCGUCAAGGCAAACGGC
AACCUGAGCAUUAUCAAACACUUUGCUUCUUUGGGCAGCGGUUUUGACAUUGUG
UCGGGCGGCGAAUUGGCACGCGUUUUGGCGGCAGGCGGCGAUGCGGCGAAAACG
AUUUUUUCCGGCGUAGGCAAAAGCGAGGCGGAAAUCGAGUUCGCGCUGAAUGCC
GGCGUAAAAUGCUUCAAUAUGGAAAGCAUCCCCGAAAUCGACCGCAUUCAGAAA
AUUGCCGCGCGUUUGGGCAAAACCGCGCCCGUCUCCCUGCGCGUCAAUCCCGAUG
UCGAUGCAAAAACCCAUCCCUACAUCUCCACAGGUCUGAAAGCCAACAAAUUCGG
CAUCGCCUACGCCGACGCGCUCGAAGCCUACCGCCAUGCCGCACAACAGCCCAAU
UUGAAAAUCAUCGGCAUCGACUGCCACAUCGGUUCGCAACUGACCGACUUAAGCC
CACUGGUCGAAGCCUGCGAACGCAUUUUGAUUUUGGUUGACGCUCUUGCCGCCGA
AGGCAUUGUUUUGGAACAUUUGGACUUAGGCGGCGGCGUCGGCAUUGUUUACAA
AGACGAAGGCGUCCCCGAUUUGGGUGCGUAUGCCCGAGCGGUUCAAAAACUGAU
GGGGACACGCCGUCUGAAACUCAUUCUUGAGCCAGGCCGCAGCUUGGUCGGCAAC
GCAGGUGCAUUGCUGACGCGCGUCGAAUUUGUCAAACACGGUGAAGAGAAAAAC
UUUGUGAUGGUCGAUGCGGCGAUGAACGAUUUGAUGCGCCCAGCCCUAUACGAU
GCCUACCACCACAUCGAAGCGGUUGAAACCAAAAACAUUGAGCCUCUGACCGCCA
ACAUCGUCGGCCCGAUUUGUGAAACCGGCGACUUCCUCGGCAAAGACCGCACCAU
CGCCUGCGAAGAAGGCGAUUUGCUGCUUAUCCGCAGCGCGGGCGCAUACGGGGCC
AGUAUGGCUAGCAAUUACAACACGCGCAACCGUGCGGCGGAGGUGUUGGUUGAC
GGCGGCGGAUACAAACUCAUCCGCCGGCGCGAAACCUUGGAACAGCAAAUGGCAA
ACGAACUCGCCUGCCUAUAA

### 35. Ngo2100: Nc_002946.2:2078991-2079314

DNA (+ strand): SEQ ID NO: 130

ATGATGACCGAAAGCGAGTTTATCCGCGCGAGCGAAGCATTATTTGAACACATCGA
AGACCAAATCGACGAAAACGGCTGGGATTTCGACTGCCGGTTTGCCGGAAACGTCC
TGACCATCGAAGCCGGAGACGGCACGCAAATCATCGTCAACCGCCACACGCCCAAC
CAAGAATTGTGGATTGCCGCAAAAAGCGGCGGCTACCATTTCGCCGAACAAAACGG
CAAATGGCTGGCAACGCGCGACAGCCGCGATTTTTACGACGTTTTAAACGAAGCCCT
GAGCGCGGCTTCGGGCGAAGCGGTTGAGATTGCCGAATTGTGA

cDNA: SEQ ID NO: 131

TCACAATTCGGCAATCTCAACCGCTTCGCCCGAAGCCGCGCTCAGGGCTTCGTTTAA
AACGTCGTAAAAATCGCGGCTGTCGCGCGTTGCCAGCCATTTGCCGTTTTGTTCGGC
GAAATGGTAGCCGCCGCTTTTTGCGGCAATCCACAATTCTTGGTTGGGCGTGTGGCG
GTTGACGATGATTTGCGTGCCGTCTCCGGCTTCGATGGTCAGGACGTTTCCGGCAAA
CCGGCAGTCGAAATCCCAGCCGTTTTCGTCGATTTGGTCTTCGATGTGTTCAAATAAT
GCTTCGCTCGCGCGGATAAACTCGCTTTCGGTCATCAT

RNA: SEQ ID NO: 132

AUGAUGACCGAAAGCGAGUUUAUCCGCGCGAGCGAAGCAUUAUUUGAACACAUC
GAAGACCAAAUCGACGAAAACGGCUGGGAUUUCGACUGCCGGUUUGCCGGAAAC
GUCCUGACCAUCGAAGCCGGAGACGGCACGCAAAUCAUCGUCAACCGCCACACGC
CCAACCAAGAAUUGUGGAUUGCCGCAAAAAGCGGCGGCUACCAUUUCGCCGAACA
AAACGGCAAAUGGCUGGCAACGCGCGACAGCCGCGAUUUUUACGACGUUUUAAAC
GAAGCCCUGAGCGCGGCUUCGGGCGAAGCGGUUGAGAUUGCCGAAUUGUGA

### 36. NGO2164: NC_002946.2:c2141372-2139807

DNA (- strand): SEQ ID NO: 133

ATGACCCAAGACAAAATCCTCATCCTCGACTTCGGTTCTCAAGTTACCCGGCTGATT
GCCCGCCGCGTGCGCGAAGCCCACGTTTACTGCGAACTGCATTCCTTCGATATGCCT
TTGGACGAAATCAAAGCCTTCAACCCCAAAGGCATCATCCTTTCCGGCGGCCCTAAT
TCTGTTTACGAATCCGACTATCAAGCCGATACCGGTATTTTTGATTTGGGCATTCCGG
TTTTGGGCATCTGCTACGGCATGCAGTTTATGGCGCACCACTTGGGTGGCGAAGTGC
AGCCCGGCAACCAGCGCGAATTCGGTTACGCGCAAGTCAAAACCATCGACAGCGGA
CTGACACGCGGCATTCAAGACGACGCGCCCAACACACTCGACGTATGGATGAGCCA
CGGCGACAAAGTGTCCAAACTGCCCGACGGTTTCGCCGTCATCGGCGATACCCCGTC
CTGCCCGATTGCAATGATGGAAAACGCCGAAAAACAATTCTACGGCATCCAGTTCC
ACCCCGAAGTTACCCACACCAAACAAGGCCGCGCCCTGTTGAACCGCTTTGTCTTGG
ATATTTGCGGCGCGCAACCGGGCTGGACGATGCCCAACTACATCGAAGAAGCCGTT
GCCAAAATCCGCGAACAAGTCGGCAGCGACGAAGTGATTTTAGGTCTGTCCGGCGG
CGTGGACTCTTCCGTAGCCGCCGCGCTGATTCACCGCGCCATCGGCGACCAACTGAC
CTGCGTGTTCGTCGATCACGGTTTGTTGCGCCTGAACGAAGGCAAAATGGTGATGGA
TATGTTCGCCCGCAACTTGGGTGTGAAAGTGATACACGTCGATGCCGAAGGGCAGTT
TATGGCGAAACTCGCCGGCGTGACCGACCCTGAGAAAAAACGCAAAATCATCGGCG
CGGAATTTATCGAAGTATTTGATGCCGAAGAGAAAAAACTCACCAACGCCAAATGG
CTGGCGCAAGGCACGATTTACCCCGACGTAATCGAATCCGCCGGTGCGAAAACCAA
AAAAGCCCACGCCATCAAATCCCACCACAACGTCGGCGGCCTGCCTGAAAATATGA
AGCTCAAACTGCTTGAGCCCTTGCGCGACTTGTTCAAAGACGAAGTGCGCGAGTTGG
GCGTGGCTTTGGGCCTGCCGCGCGAAATGGTGTACCGCCACCCCTTCCCGGGCCCCG
GTTTGGGTGTGCGCATCTTGGGCGAAGTGAAAAAAGAATACGCCGACTTGCTGCGTC
AGGCGGACGATATTTTCATCCAAGAATTACGCAATACTACCGACGAAAACGGCACG
TCTTGGTATGACCTGACCAGCCAGGCATTTGCCGTATTCCTGCCCGTCAAATCCGTC
GGCGTGATGGGCGACGGCCGCACTTACGACTACGTCGTCGCACTGCGCGCAGTCAT
CACCAGCGACTTTATGACTGCACACTGGGCAGAGCTGCCATACTCACTGCTCGGCCG
CGTGTCCAACCGCATCATCAACGAAGTCAAAGGCATCAACCGCGTGGTGTACGATG
TCAGCGGCAAACCGCCCGCCACCATCGAGTGGGAATAA

cDNA: SEQ ID NO: 134

TTATTCCCACTCGATGGTGGCGGGCGGTTTGCCGCTGACATCGTACACCACGCGGTT
GATGCCTTTGACTTCGTTGATGATGCGGTTGGACACGCGGCCGAGCAGTGAGTATGG
CAGCTCTGCCCAGTGTGCAGTCATAAAGTCGCTGGTGATGACTGCGCGCAGTGCGAC
GACGTAGTCGTAAGTGCGGCCGTCGCCCATCACGCCGACGGATTTGACGGGCAGGA
ATACGGCAAATGCCTGGCTGGTCAGGTCATACCAAGACGTGCCGTTTTCGTCGGTAG
TATTGCGTAATTCTTGGATGAAAATATCGTCCGCCTGACGCAGCAAGTCGGCGTATT
CTTTTTTCACTTCGCCCAAGATGCGCACACCCAAACCGGGGCCCGGGAAGGGGTGG
CGGTACACCATTTCGCGCGGCAGGCCCAAAGCCACGCCCAACTCGCGCACTTCGTCT
TTGAACAAGTCGCGCAAGGGCTCAAGCAGTTTGAGCTTCATATTTTCAGGCAGGCCG
CCGACGTTGTGGTGGGATTTGATGGCGTGGGCTTTTTTGGTTTTCGCACCGGCGGATT
CGATTACGTCGGGGTAAATCGTGCCTTGCGCCAGCCATTTGGCGTTGGTGAGTTTTTT
CTCTTCGGCATCAAATACTTCGATAAATTCCGCGCCGATGATTTTGCGTTTTTTCTCA
GGGTCGGTCACGCCGGCGAGTTTCGCCATAAACTGCCCTTCGGCATCGACGTGTATC
ACTTTCACACCCAAGTTGCGGGCGAACATATCCATCACCATTTTGCCTTCGTTCAGG
CGCAACAAACCGTGATCGACGAACACGCAGGTCAGTTGGTCGCCGATGGCGCGGTG
AATCAGCGCGGCGGCTACGGAAGAGTCCACGCCGCCGGACAGACCTAAAATCACTT
CGTCGCTGCCGACTTGTTCGCGGATTTTGGCAACGGCTTCTTCGATGTAGTTGGGCAT
CGTCCAGCCCGGTTGCGCGCCGCAAATATCCAAGACAAAGCGGTTCAACAGGGCGC
GGCCTTGTTTGGTGTGGGTAACTTCGGGGTGGAACTGGATGCCGTAGAATTGTTTTT
CGGCGTTTTCCATCATTGCAATCGGGCAGGACGGGGTATCGCCGATGACGGCGAAA
CCGTCGGGCAGTTTGGACACTTTGTCGCCGTGGCTCATCCATACGTCGAGTGTGTTG
GGCGCGTCGTCTTGAATGCCGCGTGTCAGTCCGCTGTCGATGGTTTTGACTTGCGCG
TAACCGAATTCGCGCTGGTTGCCGGGCTGCACTTCGCCACCCAAGTGGTGCGCCATA
AACTGCATGCCGTAGCAGATGCCCAAAACCGGAATGCCCAAATCAAAAATACCGGT
ATCGGCTTGATAGTCGGATTCGTAAACAGAATTAGGGCCGCCGGAAAGGATGATGC
CTTTGGGGTTGAAGGCTTTGATTTCGTCCAAAGGCATATCGAAGGAATGCAGTTCGC
AGTAAACGTGGGCTTCGCGCACGCGGCGGGCAATCAGCCGGGTAACTTGAGAACCG
AAGTCGAGGATGAGGATTTTGTCTTGGGTCAT

RNA: SEQ ID NO: 135

AUGACCCAAGACAAAAUCCUCAUCCUCGACUUCGGUUCUCAAGUUACCCGGCUGA
UUGCCCGCCGCGUGCGCGAAGCCCACGUUUACUGCGAACUGCAUUCCUUCGAUAU
GCCUUUGGACGAAAUCAAAGCCUUCAACCCCAAAGGCAUCAUCCUUUCCGGCGGC
CCUAAUUCUGUUUACGAAUCCGACUAUCAAGCCGAUACCGGUAUUUUUGAUUUG
GGCAUUCCGGUUUUGGGCAUCUGCUACGGCAUGCAGUUUAUGGCGCACCACUUGG
GUGGCGAAGUGCAGCCCGGCAACCAGCGCGAAUUCGGUUACGCGCAAGUCAAAAC
CAUCGACAGCGGACUGACACGCGGCAUUCAAGACGACGCGCCCAACACACUCGAC
GUAUGGAUGAGCCACGGCGACAAAGUGUCCAAACUGCCCGACGGUUUCGCCGUCA
UCGGCGAUACCCCGUCCUGCCCGAUUGCAAUGAUGGAAAACGCCGAAAAACAAUU
CUACGGCAUCCAGUUCCACCCCGAAGUUACCCACACCAAACAAGGCCGCGCCCUG
UUGAACCGCUUUGUCUUGGAUAUUUGCGGCGCGCAACCGGGCUGGACGAUGCCCA
ACUACAUCGAAGAAGCCGUUGCCAAAAUCCGCGAACAAGUCGGCAGCGACGAAGU
GAUUUUAGGUCUGUCCGGCGGCGUGGACUCUUCCGUAGCCGCCGCGCUGAUUCAC
CGCGCCAUCGGCGACCAACUGACCUGCGUGUUCGUCGAUCACGGUUUGUUGCGCC
UGAACGAAGGCAAAAUGGUGAUGGAUAUGUUCGCCCGCAACUUGGGUGUGAAAG
UGAUACACGUCGAUGCCGAAGGGCAGUUUAUGGCGAAACUCGCCGGCGUGACCGA
CCCUGAGAAAAAACGCAAAAUCAUCGGCGCGGAAUUUAUCGAAGUAUUUGAUGC
CGAAGAGAAAAAACUCACCAACGCCAAAUGGCUGGCGCAAGGCACGAUUUACCCC
GACGUAAUCGAAUCCGCCGGUGCGAAAACCAAAAAAGCCCACGCCAUCAAAUCCC
ACCACAACGUCGGCGGCCUGCCUGAAAAUAUGAAGCUCAAACUGCUUGAGCCCUU
GCGCGACUUGUUCAAAGACGAAGUGCGCGAGUUGGGCGUGGCUUUGGGCCUGCC
GCGCGAAAUGGUGUACCGCCACCCCUUCCCGGGCCCCGGUUUGGGUGUGCGCAUC
UUGGGCGAAGUGAAAAAAGAAUACGCCGACUUGCUGCGUCAGGCGGACGAUAUU
UUCAUCCAAGAAUUACGCAAUACUACCGACGAAAACGGCACGUCUUGGUAUGACC
UGACCAGCCAGGCAUUUGCCGUAUUCCUGCCCGUCAAAUCCGUCGGCGUGAUGGG
CGACGGCCGCACUUACGACUACGUCGUCGCACUGCGCGCAGUCAUCACCAGCGAC
UUUAUGACUGCACACUGGGCAGAGCUGCCAUACUCACUGCUCGGCCGCGUGUCCA
ACCGCAUCAUCAACGAAGUCAAAGGCAUCAACCGCGUGGUGUACGAUGUCAGCGG
CAAACCGCCCGCCACCAUCGAGUGGGAAUAA

### 37. NGO2173: NC_002946.2:c2149065-2148886

DNA (- strand): SEQ ID NO: 136

ATGGCCGTTCAACAAAACAAAAAATCCCCTTCCAAACGCGGTATGCACCGTTCGCA
CGACGCACTGACCGCGCCCGCACTGTTTGTCGACAGCACAACCGGCGAAGTACACC
GCCCGCACCACATCTCCCCCAACGGTATGTACCGCGGCCGCAAAGTGGTCAAAGCC
AAAGGCGAATAA

cDNA: SEQ ID NO: 137

TTATTCGCCTTTGGCTTTGACCACTTTGCGGCCGCGGTACATACCGTTGGGGGAGAT
GTGGTGCGGGCGGTGTACTTCGCCGGTTGTGCTGTCGACAAACAGTGCGGGCGCGGT
CAGTGCGTCGTGCGAACGGTGCATACCGCGTTTGGAAGGGGATTTTTTGTTTTGTTG
AACGGCCAT

RNA: SEQ ID NO: 138

AUGGCCGUUCAACAAAACAAAAAAUCCCCUUCCAAACGCGGUAUGCACCGUUCGC
ACGACGCACUGACCGCGCCCGCACUGUUUGUCGACAGCACAACCGGCGAAGUACA
CCGCCCGCACCACAUCUCCCCCAACGGUAUGUACCGCGGCCGCAAAGUGGUCAAA
GCCAAAGGCGAAUAA

### 38. NGO2174: NC_002946.2:c2149599-2149099

DNA (- strand): SEQ ID NO: 139

ATGTCAGACCCTAATTTGATTGACCCGGAAATTTTTGCCGCCGAAAGGCAGAACCTG
CAAGGCAGTTTTCTGCTGGAAGAATTGGACGAGCGAGTCAGTTTGCACGATTATCCC
GCCGACAGGCGGAACAAAATATCGTTTACACTGACCGGCGGTCGCGACCGGCTGCA
ACGCCTGTTCCTCGACCTGAACGTCAAAGCCGATATGCCCCTGATTTGCCAGAGATG
TATCAAACCCATGCCGTTCATGCTCGATGAAAGCAGCCGTATCATCCTGTTTTCCGA
CGAAGAGTCCTTGGACGAATCCATGCTTGCCGACGAAGAACTCGAAGGCATACTGA
TTGAAAAAGAACTCGACGTGCGCGCATTGGTAGAAGACCAAATCCTGATGTCCCTG
CCCTTTTCGCCGCGACACGGACACTGCGGCAATACCCTTCCGGAATCCGCCAACCAA
GACAAACCCAACCCCTTTGCTGTTTTGGCGGGTTTGAAAAGCAGTTAA

cDNA: SEQ ID NO: 140

TTAACTGCTTTTCAAACCCGCCAAAACAGCAAAGGGGTTGGGTTTGTCTTGGTTGGC
GGATTCCGGAAGGGTATTGCCGCAGTGTCCGTGTCGCGGCGAAAAGGGCAGGGACA
TCAGGATTTGGTCTTCTACCAATGCGCGCACGTCGAGTTCTTTTTCAATCAGTATGCC
TTCGAGTTCTTCGTCGGCAAGCATGGATTCGTCCAAGGACTCTTCGTCGGAAAACAG
GATGATACGGCTGCTTTCATCGAGCATGAACGGCATGGGTTTGATACATCTCTGGCA
AATCAGGGGCATATCGGCTTTGACGTTCAGGTCGAGGAACAGGCGTTGCAGCCGGT
CGCGACCGCCGGTCAGTGTAAACGATATTTTGTTCCGCCTGTCGGCGGGATAATCGT
GCAAACTGACTCGCTCGTCCAATTCTTCCAGCAGAAAACTGCCTTGCAGGTTCTGCC
TTTCGGCGGCAAAAATTTCCGGGTCAATCAAATTAGGGTCTGACAT

RNA: SEQ ID NO: 141

AUGUCAGACCCUAAUUUGAUUGACCCGGAAAUUUUUGCCGCCGAAAGGCAGAACC
UGCAAGGCAGUUUUCUGCUGGAAGAAUUGGACGAGCGAGUCAGUUUGCACGAUU
AUCCCGCCGACAGGCGGAACAAAAUAUCGUUUACACUGACCGGCGGUCGCGACCG
GCUGCAACGCCUGUUCCUCGACCUGAACGUCAAAGCCGAUAUGCCCCUGAUUUGC
CAGAGAUGUAUCAAACCCAUGCCGUUCAUGCUCGAUGAAAGCAGCCGUAUCAUCC
UGUUUUCCGACGAAGAGUCCUUGGACGAAUCCAUGCUUGCCGACGAAGAACUCGA
AGGCAUACUGAUUGAAAAAGAACUCGACGUGCGCGCAUUGGUAGAAGACCAAAU
CCUGAUGUCCCUGCCCUUUUCGCCGCGACACGGACACUGCGGCAAUACCCUUCCG
GAAUCCGCCAACCAAGACAAACCCAACCCCUUUGCUGUUUUGGCGGGUUUGAAAA
GCAGUUAA

### SEQUENCES FOR TRNA

### 1. NGO_t01: NC_002946.2:c14067-13982

DNA (- strand): SEQ ID NO: 142

GCCGACATGGTGAAATTGGTAGACACGCTATCTTGAGGGGGTAGTGGCCGTAGGCT
GTGCGAGTTCAAATCTCGCTGTCGGCACCA

cDNA: SEQ ID NO: 143

TGGTGCCGACAGCGAGATTTGAACTCGCACAGCCTACGGCCACTACCCCCTCAAGAT
AGCGTGTCTACCAATTTCACCATGTCGGC

RNA: SEQ ID NO: 144

GCCGACAUGGUGAAAUUGGUAGACACGCUAUCUUGAGGGGGUAGUGGCCGUAGG
CUGUGCGAGUUCAAAUCUCGCUGUCGGCACCA

### 2. NGO_t12: NC_002946.2:454725-454812

DNA (+ strand): SEQ ID NO: 145

GGAAGCGTGGCAGAGCGGTTTAATGCAACGGTCTTGAAAACCGTCGAGGGTTGATA
GCCCTCCGTGAGTTCGAATCTCACCGCTTCCG

cDNA: SEQ ID NO: 146

CGGAAGCGGTGAGATTCGAACTCACGGAGGGCTATCAACCCTCGACGGTTTTCAAG
ACCGTTGCATTAAACCGCTCTGCCACGCTTCC

RNA: SEQ ID NO: 147

GGAAGCGUGGCAGAGCGGUUUAAUGCAACGGUCUUGAAAACCGUCGAGGGUUGA
UAGCCCUCCGUGAGUUCGAAUCUCACCGCUUCCG

### 3. NGO_t14: NC_002946.2:793319-793402

DNA (+ strand): SEQ ID NO: 148

GCCCGGGTGGCGGAATTGGTAGACGCGCCAGCTTCAGGTGCTGGTATCCTCACGGGT
ATGGAAGTTCGAGTCTTCTCCCGGGCA

cDNA: SEQ ID NO: 149

TGCCCGGGAGAAGACTCGAACTTCCATACCCGTGAGGATACCAGCACCTGAAGCTG
GCGCGTCTACCAATTCCGCCACCCGGGC

RNA: SEQ ID NO: 150

GCCCGGGUGGCGGAAUUGGUAGACGCGCCAGCUUCAGGUGCUGGUAUCCUCACGG
GUAUGGAAGUUCGAGUCUUCUCCCGGGCA

### 4. NGO_t15: NC_002946.2:793444-793531

DNA (+ strand): SEQ ID NO: 151

AGAGAGGTGGATGAGTGGTTTAAGTCGCACGCCTGGAAAGCGTGTATACGTGAATA
GCGTATCGAGGGTTCGAATCCCTTCCTCTCTG

cDNA: SEQ ID NO: 152

CAGAGAGGAAGGGATTCGAACCCTCGATACGCTATTCACGTATACACGCTTTCCAGG
CGTGCGACTTAAACCACTCATCCACCTCTCT

RNA: SEQ ID NO: 153

AGAGAGGUGGAUGAGUGGUUUAAGUCGCACGCCUGGAAAGCGUGUAUACGUGAA
UAGCGUAUCGAGGGUUCGAAUCCCUUCCUCUCUG

### 5. NGO_t37: NC_002946.2:cl629552-1629481

DNA (- strand): SEQ ID NO: 228

CTCGCCATAGTTCAACGGATAGAACGTATGCCTCCTAAGCGTAAAATACAGGTTCGA
TTCCTGTTGGCGAGG

cDNA: SEQ ID NO: 229

CCTCGCCAACAGGAATCGAACCTGTATTTTACGCTTAGGAGGCATACGTTCTATCCG
TTGAACTATGGCGAG

RNA: SEQ ID NO: 230

CUCGCCAUAGUUCAACGGAUAGAACGUAUGCCUCCUAAGCGUAAAAUACAGGUUC
GAUUCCUGUUGGCGAGG

### ANNEX C

### Expected Exemplary RNA Markers of N. Meningitides

### PorB

### NC_003112.2:2157529-2158524 Neisseria Meningitides MC58

DNA(+)strand: SEQ ID NO: 154

ATGAAAAAATCCCTGATTGCCCTGACTTTGGCAGCCCTTCCTGTTGCAGCAATGGCTGACGT
TACCCTGTACGGCACCATCAAAGCCGGCGTAGAAACTTCCCGCTCTGTATTTCACCAGAACG
GCCAAGTTACTGAAGTTACAACCGCTACCGGCATCGTTGATTTGGGTTCGAAAATCGGCTTC
AAAGGCCAAGAAGACCTCGGTAACGGCCTGAAAGCCATTTGGCAGGTTGAGCAAAAAGCAT
CTATCGCCGGTACTGACTCCGGTTGGGGCAACCGCCAATCCTTCATCGGCTTGAAAGGCGGC
TTCGGTAAATTGCGCGTCGGTCGTTTGAACAGCGTCCTGAAAGACACCGGCGACATCAATCC
TTGGGATAGCAAAAGCGACTATTTGGGTGTAAACAAAATTGCCGAACCCGAGGCACGCCTC
ATTTCCGTACGCTACGATTCTCCCGAATTTGCCGGCCTCAGCGGCAGCGTACAATACGCGCT
TAACGACAATGCAGGCAGACATAACAGCGAATCTTACCACGCCGGCTTCAACTACAAAAAC
GGTGGCTTCTTCGTGCAATATGGCGGTGCCTATAAAAGACATCATCAAGTGCAAGAGGGCTT
GAATATTGAGAAATACCAGATTCACCGTTTGGTCAGCGGTTACGACAATGATGCCCTGTACG
CTTCCGTAGCCGTACAGCAACAAGACGCGAAACTGACTGATGCTTCCAATTCGCACAACTCT
CAAACCGAAGTTGCCGCTACCTTGGCATACCGCTTCGGCAACGTAACGCCCCGAGTTTCTTA
CGCCCACGGCTTCAAAGGTTTGGTTGATGATGCAGACATAGGCAACGAATACGACCAAGTG
GTTGTCGGTGCGGAATACGACTTCTCCAAACGCACTTCTGCCTTGGTTTCTGCCGGTTGGTTG
CAAGAAGGCAAAGGCGAAAACAAATTCGTAGCGACTGCCGGCGGTGTCGGTCTGCGCCACA
AATTCTAA

cDNA: SEQ ID NO: 155

ATGAAAAAATCCCTGATTGCCCTGACTTTGGCAGCCCTTCCTGTTGCAGCAATGGCTGACGT
TACCCTGTACGGCACCATCAAAGCCGGCGTAGAAACTTCCCGCTCTGTATTTCACCAGAACG
GCCAAGTTACTGAAGTTACAACCGCTACCGGCATCGTTGATTTGGGTTCGAAAATCGGCTTC
AAAGGCCAAGAAGACCTCGGTAACGGCCTGAAAGCCATTTGGCAGGTTGAGCAAAAAGCAT
CTATCGCCGGTACTGACTCCGGTTGGGGCAACCGCCAATCCTTCATCGGCTTGAAAGGCGGC
TTCGGTAAATTGCGCGTCGGTCGTTTGAACAGCGTCCTGAAAGACACCGGCGACATCAATCC
TTGGGATAGCAAAAGCGACTATTTGGGTGTAAACAAAATTGCCGAACCCGAGGCACGCCTC
ATTTCCGTACGCTACGATTCTCCCGAATTTGCCGGCCTCAGCGGCAGCGTACAATACGCGCT
TAACGACAATGCAGGCAGACATAACAGCGAATCTTACCACGCCGGCTTCAACTACAAAAAC
GGTGGCTTCTTCGTGCAATATGGCGGTGCCTATAAAAGACATCATCAAGTGCAAGAGGGCTT
GAATATTGAGAAATACCAGATTCACCGTTTGGTCAGCGGTTACGACAATGATGCCCTGTACG
CTTCCGTAGCCGTACAGCAACAAGACGCGAAACTGACTGATGCTTCCAATTCGCACAACTCT
CAAACCGAAGTTGCCGCTACCTTGGCATACCGCTTCGGCAACGTAACGCCCCGAGTTTCTTA
CGCCCACGGCTTCAAAGGTTTGGTTGATGATGCAGACATAGGCAACGAATACGACCAAGTG
GTTGTCGGTGCGGAATACGACTTCTCCAAACGCACTTCTGCCTTGGTTTCTGCCGGTTGGTTG
CAAGAAGGCAAAGGCGAAAACAAATTCGTAGCGACTGCCGGCGGTGTCGGTCTGCGCCACA
AATTCTAA

RNA: SEQ ID NO: 156

UUAGAAUUUGUGGCGCAGACCGACACCGCCGGCAGUCGCUACGAAUUUGUUUUCGCCUUU
GCCUUCUUGCAACCAACCGGCAGAAACCAAGGCAGAAGUGCGUUUGGAGAAGUCGUAUUC
CGCACCGACAACCACUUGGUCGUAUUCGUUGCCUAUGUCUGCAUCAUCAACCAAACCUUU
GAAGCCGUGGGCGUAAGAAACUCGGGGCGUUACGUUGCCGAAGCGGUAUGCCAAGGUAG
CGGCAACUUCGGUUUGAGAGUUGUGCGAAUUGGAAGCAUCAGUCAGUUUCGCGUCUUGU
UGCUGUACGGCUACGGAAGCGUACAGGGCAUCAUUGUCGUAACCGCUGACCAAACGGUGA
AUCUGGUAUUUCUCAAUAUUCAAGCCCUCUUGCACUUGAUGAUGUCUUUUAUAGGCACC
GCCAUAUUGCACGAAGAAGCCACCGUUUUUGUAGUUGAAGCCGGCGUGGUAAGAUUCGC
UGUUAUGUCUGCCUGCAUUGUCGUUAAGCGCGUAUUGUACGCUGCCGCUGAGGCCGGCAA
AUUCGGGAGAAUCGUAGCGUACGGAAAUGAGGCGUGCCUCGGGUUCGGCAAUUUUGUUU
ACACCCAAAUAGUCGCUUUUGCUAUCCCAAGGAUUGAUGUCGCCGGUGUCUUUCAGGACG
CUGUUCAAACGACCGACGCGCAAUUUACCGAAGCCGCCUUUCAAGCCGAUGAAGGAUUGG
CGGUUGCCCCAACCGGAGUCAGUACCGGCGAUAGAUGCUUUUUGCUCAACCUGCCAAAUG
GCUUUCAGGCCGUUACCGAGGUCUUCUUGGCCUUUGAAGCCGAUUUUCGAACCCAAAUCA
ACGAUGCCGGUAGCGGUUGUAACUUCAGUAACUUGGCCGUUCUGGUGAAAUACAGAGCG
GGAAGUUUCUACGCCGGCUUUGAUGGUGCCGUACAGGGUAACGUCAGCCAUUGCUGCAAC
AGGAAGGGCUGCCAAAGUCAGGGCAAUCAGGGAUUUUUUCAU

### rpmB

### >NC_003112.2:332567-332800 Neisseria Meningitidis MC58 - on the (-)strand

DNA (+)strand SEQ ID NO: 157

TTAAGCTTCGCCGCGAGCACGCAAATCAGCCAATACGACATCAATGCCTACTTTGTCGATGG
TACGCAGTGCAGCGTTGGAAACGCGCAGGCGAACCCAGCGGTTTTCACTTTCTACCCAAAAA
CGACGTGATTGCAAGTTGGGCAAAAAACGGCGTTTGGTTTTGTTGTTGGCGTGCGATACGTT
GTTGCCGGACATCGGGCGTTTGCCGGTCACTTTGCAAACTCGTGCCAT

Cdna SEQ ID NO: 158

ATGGCACGAGTTTGCAAAGTGACCGGCAAACGCCCGATGTCCGGCAACAACGTATCGCACG
CCAACAACA
AAACCAAACGCCGTTTTTTGCCCAACTTGCAATCACGTCGTTTTTGGGTAGAAAGTGAAAAC
CGCTGGGT
TCGCCTGCGCGTTTCCAACGCTGCACTGCGTACCATCGACAAAGTAGGCATTGATGTCGTAT
TGGCTGAT
TTGCGTGCTCGCGGCGAAGCTTAA

RNA SEQ ID NO: 159

UUAAGCUUCGCCGCGAGCACGCAAAUCAGCCAAUACGACAUCAAUGCCUACUUUGUCGAU
GGUACGCAGUGCAGCGUUGGAAACGCGCAGGCGAACCCAGCGGUUUUCACUUUCUACCCA
AAAACGACGUGAUUGCAAGUUGGGCAAAAAACGGCGUUUGGUUUUGUUGUUGGCGUGCG
AUACGUUGUUGCCGGACAUCGGGCGUUUGCCGGUCACUUUGCAAACUCGUGCCAU

### ANNEX D

>ng_165_porB SEQ ID NO: 178

ATGAAAAAATCCCTGATTGCCCTGACTTTGGCAGCCCTTCCTGTTGCGGCAACGGCCGAT
GTTACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_166_porB SEQ ID NO: 179

ATGAAAAAATCCCTGATTGCCCTGACTTTGGCAGCCCTTCCTGTTGCGGCAACGGCCGAT
GTTACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCCTTGTTCCAA
AGATACGGCGAAGGCACTAAAAAAATGGAATACGATAATCAATTTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_167_porB SEQ ID NO: 180

ATGAAAAAATCCCTGATTGCCCTGACTTTGGCAGCCCTTCCTGTTGCGGCAACGGCCGAT
GTTACCCTGTACGGCGCCATCAAAGCCGGCGTACAAACTTACCGTTCTGTAGAACATCGG
GAAGGCAAAGTAGTTGGCGTGGGAACCGGCAGCGAAATCTCCGACTTCGGTTCAAAAATC
GGCTTCAAAGGTCAAGAAGACCTCGGCAACGGCCTGAAGGCCGTTTGGCAGTTGGAACAA
GGCGCCTCCGTCGCCGGCGCTAACACCGGCTGGGGCAACAAACAATCCTTCGTCGGCTTG
AAAGGCGGCTTCGGCACCATCCGCGTCGGCAGCCTGAACAGCCCCCTGAAAAACACCGGT
GCCAACGTCAATGCTTGGGAATCCGGCAAATATACCGGCGAGCTTCTGGAAATCAGCAAA
ATGGCCGGACGGGAACACCGCTACCTGTCCGCACGCTACGATTCTCCCGAATTTGCCGGC
TTCAGCGGCAGCGTACAATACGCACCTAAAGGTAATTCAGGCTCAAACGGCGAATCTTAC
CACGTTGGCTTGAACTACCGAAACAGCGGCTTCTTCGCACAATACGCCGGCTTGTTCCAA
AGATACGGCGAAGGCACTAAAAAAATCGAATACGATGATCAAACTTATAGTATGCCCAGT
CTGTTTGTTGAAAAACTGCAAGTTCACCGTTTGGTAGGCGGTTACGACAATAATGCCCTG
TACGTTTCCGTAGCCGCACAACAACAAGATGCCAAATTGTATGGAGCAACGAGGGTTAAT
TCGCACAACTCTCAAACCGAAGTTGCCGCTACCGCGGCATACCGTTTCGGCAATGTAACG
CCCCGCGTTTCTTACGCCCACGGCTTCAAAGGCACTGTTGATGATGCAAACCACGACAAT
ACTTATGACCAAGTGGTTGTCGGTGCGGAATACGACTTCTCCAAACGCACTTCTGCCTTG
GTTTGCTGCCGGCTGGTTGCAAGAAGGCAAAGGCGCAGACAAAATCGTATCGACTGCCAGC
GCCGTCGTTCTGCGCCACAAATTCTAA

>ng_168_porB SEQ ID NO: 181

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAGTTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_169_porB SEQ ID NO: 182

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCEGAAAGCCAGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATCTGAATACCTATGGTCAAACTTAIAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAACGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_170_porB SEQ ID NO: 183

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_171_porB SEQ ID NO: 184

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_172_porB SEQ ID NO: 185

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAGTTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_173_porB SEQ ID NO: 186

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAACGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_174_porB SEQ ID NO: 187

ATGAAAAAATCCCTGATTGCCCTGACTTTGGCAGCCCTTCCTGTTGCGGCAACGGCCGAT
TCACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_175_porB SEQ ID NO: 188

ATGAAAAAATCCCTGATTGCCCTGACTTTGGCAGCCCTTCCTGTTGCGGCAACGGCCGAT
GTTACCCTGTACGGCGCCATCAAAGCCGGCGTACAAACTTACCGTTCTAGAACATCGG
GAAGGCAAAGTAGTTGGCGTGGGAACCGGCAGCGAAATCTCCGACTTCGGTTCAAAAATC
GGCTTCAAAGGTCAAGAAGACCTCGGCAACGGCCTGAAGGCCGTTTGGCAGTTGGAACAA
GGCGCCTCCGTCGCCGGCGCTAACACCGGCTGGGGCAACAAACAATCCTTCGTCGGCTTG
AAAGGCGGCTTCGGCACCATCCGCGTCGGCAGCCTGAACAGCCCCCTGAAAAACACCGGT
GCCAACGTCAATGCTTGGGAATCCGGCAAATATACCGGCGAGCTTCTGGAAATCAGCAAA
ATGGCCGGACGGGAACACCGCTACCTGTCCGCACGCTACGATTCTCCCGAATTTGCCGGC
TTCAGCGGCAGCGTACAATACGCACCTAAAGGTAATTCAGGCTCAAACGGCGAATCTTAC
CACGTTGGCTTGAACTACCGAAACAGCGGCTTCTTCGCACAATACGCCGGCTTGTTCCAA
AGATACGGCGAAGGCACTAAAAAAATCGAATACGATGATCAAACTTATAGTATGCCCAGT
CTGTTTGTTGAAAAACTGCAAGTTCACCGTTTGGTAGGCGGTTACGACAATAATGCCCTG
TACGTTTCCGTAGCCGCACAACAACAAGATGCCAAATTGTATGGAGCAACGAGGGTTAAT
TCGCACAACTCTCAAACCGAAGTTGCCGCTACCGCGGCATACCGTTTCGGCAATGTAACG
CCCCGCGTTTCTTACGCCCACGGCTTCAAAGGCACTGTTGATGATGCAAACCACGACAAT
ACTTATGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_176_porB SEQ ID NO: 189

ATGAAAAAATCCCTGATTGCCCTGACTTTGGCAGCCCTTCCTGTTGCGGCAACGGCCGAT
GTCACCCTGTACGGCGGCATCAAAGCCGGCGTACAAACTTACCGTTCTGTAGAACATCGG
GAAGGCAAAGTAGTTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_177_porB SEQ ID NO: 190

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAGTTGGCGTGGGAACCGGCAGCGAAATCTCCGACTTCGGTTCAAAAATC
GGCTTCAAAGGCCAAGAAGACCTCGGCAACGGCCTGAAGGCCGTTTGGCAGTTGGAACAA
GGCGCCTCCGTCGCCGGCACTAACACCGGCTGGGGCAACAAACAATCCTTCGTCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATCGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_178_porB SEQ ID NO: 191

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_179_porB SEQ ID NO: 192

ATGAAAAAATCCCTGATTGCCCTGACTTTGGCAGCCCTTCCTGTTGCGGCAACGGCCGAT
GTTACCCTGTACGGCGCCATCAAAGCCGGCGTACAAACTTACCGTTCTGTAGAACATCGG
GAAGGCAAAGTAGTTGGCGTGGAAACCGGCAGCGAAATCTCCGACTTCGGTTCAAAAATC
GGCTTCAAAGGTCAAGAAGACCTCGGCAACGGCCTGAAGGCCGTTTGGCAGTTGGAACAA
GGTGCCTCCGTCGCCGGCACTAACACCGGCTGGGGCAACAAACAATCCTTCGTCGGCTTG
AAAGGCGGCTTCGGCACCATCCGCGTCGGCAGCCTGAACAGCCCCCTGAAAAACACCGGT
GCCAACGTCAATGCTTGGGAATCCGGCAAATATACCGGCGAGTTTCTGGAAATCAGCAAA
ATGGCCAGACGGGAACACCGCTACCTGTCCGCACGCTACGATTCTCCCGAATTTGCCGGC
TTCAGCGGCAGCGTACAATACGCACCTAAAGATAATTCAGGCTCAAACGGCGAATCTTAC
CACGTTGGCTTGAACTACCGAAACGGCGGCTTCTTCGCACAATACGCCGGCTTGTTCCAA
AGATACGGCGAAGGCACTAAAAAAATCGAATACGCTGGTCAATATTATAGTATCCCCAGC
CTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAACATGGAGTGCTAAT
TCGCACAACTCTCAAACCGAAGTTGCCGCTACCGCGGCATACCGTTTCGGCAACTTAACG
CCCCGCGTTTCTTACGCCCACGGCTTCAAAGGCTCTGTTCATAGTGCAGACTACGACAAT
ACTTATGACCAAGTGGTTGTCGGTGCGGAATACGACTTCTCCAAACGCACTTCTGCCTTG
GTTTCTGCCGGCTGGTTGCAAGAAGGCAAAGGCGCAGAAAAAGTCGTATCGACTGCCAGC
GCCGTCGTTCTGCGCCACAAATTCTAA

>ng_180_porB SEQ ID NO: 193

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAGTTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_181_porB SEQ ID NO: 194

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAGTTGACGTGGGAACCGGCAGCGAAATCTCCGACTTCGGTTCAAAAATC
GGCTTCAAAGGCCAAGAAGACCTCGGCAACGGCCTGAAGGCCGTTTGGCAGTTGGAACAA
GGCGCCTCCGTCGCCGGCGCTAACAGCGGCTGGGGCAACAAACAATCCTTCGTCGGCTTG
AAGGGCGGCTTCGGCACCATCCGCGCCGGTAGCCTGAACAGCCCCCTGAAAAACACCGGC
AGCAAAGTCAATGCTTGGGAATCCGGCAAATTTACCGGCAGTGTGCTGAAAATCAGCGGA
ATGGCCGAACGGGAACACCGCTACCTGTCCGCACGCTACGATTCTCCCGAATTTGCCGGC
TTCAGCGGCAGCGTACAATACGCACCTAAAGACAATTCAGGCTCAAACGGCGAATCTTAC
CACGTTGGCTTGAACTACCAAAACAGCGGCTTCTTCGCACAATACGCCGGCTTGTTCCAA
AGATACGGCGAAGGCACTAAAAAAATCGAATACGATGGICAAACTIATAGTATGCCCAGT
CTGTTTGTTGAAAAACTGCAAGTTCACCGTTTGGTAGGCGGTTACGACAATAATGCCCTG
TACGCCTCCGTAGCCGCACAACAACAAGATGCCAAATTGTATGGAACATGGAGTGCTAAT
TCGCACAACTCTCAAACCGAAGTTGCCGCTACCGTGGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_182_porB SEQ ID NO: 195

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGAAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAATCTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_183_porB SEQ ID NO: 196

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_184_porB SEQ ID NO: 197

ATGAAAAAATCCCTGATTGCCCTGACTTTGGCAGCCCTTCCTGTTGCGGCAACGGCCGAT
TCACCCTGTACGGCGCCATCAAAGCCGGCGTACAAACTTACCGTTCTGTAGAACATCGG
GAAGGCAAAGTAGTTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_185_porB SEQ ID NO: 198

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTACTTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_186_porB SEQ ID NO: 199

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_187_porB SEQ ID NO: 200

ATGAAAAAATCCCTGATTGCCCTGACTTTGGCAGCCCTTCCTGTTGCGGCAACGGCCGAT
GTTACCCTGTACGGCGCCATCAAAGCCGGCGTACAAACTTACCGTTCTGTAGAACATCGG
GAAGGCAAAGTAGTTGGCGTGGGAACCGGCAGCGAAATCTCCGACTTCGGTTCAAAAATC
GGCTTCAAAGGTCAAGAAGACCTCGGCAACGGCCTGAAGGCCGTTTGGCAGTTGGAACAA
GGCGCCTCCGTCGCCGGCACTAACACCGGCTGGGGCAACAAACAATCCTTCGTCGGCTTG
AAAGGCGGCTTCGGCACCATCCGCGTCGGCAGCCTGAACAGCCCCCIGAAAAACACCGGT
GCCAACGTCAATGCTTGGGAAICCGGCAAATATACCGGCGAGCTTCTGGAAAICAGCAAA
ATGGCCGGACGGGAACACCGCTACCTGTCCGCACGCTACGATTCTCCCGAATTTGCCGGC
TTCAGCGGCAGCGTACAATACGCACCTAAAGGTAATTCAGGCTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGCGGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_188_porB SEQ ID NO: 201

ATGAAAAAATCCCTGATTGCCCTGACTTTGGCAGCCCTTCCTGTTGCGGCAACGGCCGAT
TCACCCTGTACGGCGGCATCAAAGCCGGCGTACAAACTTACCGTTCTGTAGAACATCGG
GAAGGCAAAGTAGTTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_189_porB SEQ ID NO: 202

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_190_porB SEQ ID NO: 203

ATGAAAAAATCCCTGATTGCCCTGACTTTGGCAGCCCTTCCTGTTGCGGCAACGGCCGAT
GTCACCCTGTACGGCACCATCAAAGCCGGCGTACAAACTTACCGTTCTGTAGAACATACA
AAAGGCAAGGTAAGTAAAGTGGAAACCGGCAGCGAAATCGCCGACTTCGGTTCAAAAATC
GGCTTCAAAGGCCAAGAAGACCTCGGCAACGGCCTGAAGGCCGTTTGGCAGTTGGAACAA
GGTGCCTCCGTCGCCGGCACTAACACCGGCTGGGGCAACAAACAATCCTTCGTCGGCTTG
AAGGGCGGCTTCGGTACCATCCGCGCCGGTAGCCTGAACAGCCCCCTGAAAAACACCAAG
GACAACGTCAATGCTTGGGAATCCGGCAAATTTACCGGCGATGTGCTGGAAATCAGCGGA
ATGGCCAAACGGGAACACCGCTACCTGTCCGTACGCTACGATTCTCCCGAATTTGCCGGC
TTCAGCGGCAGCGTACAATACGCACCTAAAGATAATTCAGGCTCAAACGGCGAATCTTAC
CACGTTGGCTTGAACTACCGAAACGGCGGCTTCTTCGCACAATACGCCGGCTTGTTCCAA
AGATACGGCGAAGGCACTAAAAAAATCGAATACGATAATCAATTTTAATAGTGTCCCCAGC
CTGTCTGTTGAAAAACTGCAAGTTCACCGTTTGGTCGGCGGTTACGACAATAATGCCCTG
TACGCCTCCGTAGCCGCACAACAACAAGATGCCAAATTGTATGGAACATGGCGTGCTAAT
TCGCACAACTCTCAAACCGAAGTTGCTGCTACCGCGGCATACCGTTTCGGCAACTTAACG
CCCCGCGTTTCTTACGCCCACGGCTTCAAAGGCTCTGTTCATAGTGCAGACTACGACAAT
ACTTATGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_191_porB SEQ ID NO: 204

ATGAAAAAATCCCTGATTGCCCTGACTTTGGCAGCCCTTCCTGTTGCGGCAACGGCCGAT
GTCACCCTGTACGGCGGCATCAAAGCCGGCGTACAAACTTACCGTTCTGTAGAACATCGG
GAACTGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_192_porB SEQ ID NO: 205

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAGTTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_193_porB SEQ ID NO: 206

ATGAAAAAATCCCTGATTGCCCTGACTTTGGCAGCCCTTCCTGTTGCGGCAACGGCCGAT
GTCACCCTGTACGGCACCATCAAAGCCGGCGTACAAACTTACCGTTCTGTAGAACATACA
AAAGGCAAGGTAAGTAAAGTGGAAACCGGCAGCGAAATCGCCGACTTCGGTTCAAAAATC
GGCTTCAAAGGCCAAGAAGACCTCGGCAACGGCCTGAAGGCCGTTTGGCAGTTGGAACAA
GGTGCCTCCGTCGCCCGGCACTAACACCGGCTCTGGGGCAACAAACAATCCTTCGTCGGCTTG
AAGGGCGGCTTCGGTACCATCCGCGCCGGTAGCCTGAACAGCCCCCTGAAAAACACCAAG
GACAACGTCAATGCTTGGGAATCCGGCAAATTTACCGGCGATGTGCTGGAAATCAGCGGA
ATGGCCAAACGGGAACACCGCTACCTGTCCGTACGCTACGATTCTCCCGAATTTGCCGGC
TTCAGCGGCAGCGTACAATACGCACCTAAAGATAATTCAGGCTCAAACGGCGAATCTTAC
CACGTTGGCTTGAACTACCGAAACGGCGGCTTCTTCGCACAATACGCCGGCTTGTTCCAA
AGATACGGCGAAGGCACTAAAAAAATCGAATACAATAATCAATTTTATAGTGTCCCCAGC
CTGTCTGTTGAAAAACTGCAAGTTCACCGTTTGGTCGGCGGTTACGACAATAATGCCCTG
TACGCCTCCGTAGCCGCACAACAACAAGATGCCAAATTGTATGGAACATGGCGTGCTAAT
TCGCACAACTCTCAAACCGAAGTTGCTGCTACCGCGGCATACCGTTTCGGCAACTTAACG
CCCCGCGTTTCTTACGCCCACGGCTTCAAAGGCTCTGTTCATAGTGCAGACTACGACAAT
ACTTATGACCAAGTGGTTGTCGGTGCGGAATACGACTTCTCCAAACGCACTTCTGCCTTG
GTTTCTGCCGGCTGGTTGCAAGCAGGCAAAGGCGCAGACAAAATCGTATCGACTGCCAGC
GCCGTCGTTCTGCGCCACAAATTCTAA

>ng_194_porB SEQ ID NO: 207

ATGAAAAAATCCCTGATTGCCCTGACTTTGGCAGCCCTTCCTGTTGCGGCAACGGCCGAT
GTTACCCTGTACGGCGCCATCAAAGCCGGCGTACAAACTTACCGTTCTGTAGAACATCGG
GAAGGCAAAGTAGTTGGCGTGGAAACCGGCAGCGAAATCTCCGACTTCGGTTCAAAAATC
GGCTTCAAAGGTCAAGAAGACCTCGGCAACGGCCTGAAGGCCGTTTGGCAGTTGGAACAA
GGTGCCTCCGTCGCCGGCACTAACACCGGCTGGGGCAACAAACAATCCTTCGTCGGCTTG
AAGGGCGGCTTCGGTACCATCCGCGCCGGTAGCCTGAACAGCCCCCTGAAAAACACCAAC
GCCAACGTCAATGCTTGGGAATCCGGCAAATTTACCGGCAATGTGCTGGAAATCAGCAAA
ATGGCCGAACGGGAACACCGCTACCTGTCCGTACGCTACGATTCTCCCGAATTTGCCGGC
TTCAGCGGCAGCGTACAATACGCACCTAAAGATAATTCAGGCTCAAACGGCGAATCTTAC
CACGTTGGCTTGAACTACCGAAACGGCGGCTTCTTCGCACAATACGCCGGCTTGTTCCAA
AGATACGGCGAAGGCACTAAAAAAATCGAATACGATAATCAATCTTATAGTATCCCCAGC
CTGTTTGTTGAAAAACTGCAAGTTCACCGTTTGGTAGGCGGTTACGACAATAATGCCCTG
TACGTCTCCGTAGCCGCACAACAACAAGATGCCAAATTGTATGGATCAATGAGCGGTAAT
TCGCACAACTCTCAAACCGAAGTTGCCGCTACCGCGGCATACCGTTTCGGCAACGTAACG
CCCCGCGTTTCTTACGCCCACGGCTTCAAAGGCAGTGTTGATAGTGCAAACCACGACAAT
ACTTATGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_195_porB SEQ ID NO: 208

ATGAAAAAATCCCTGATTGCCCTGACTTTGGCAGCCCTTCCTGTTGCGGCAACGGCCGAT
GTTACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATAATCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_196_porB SEQ ID NO: 209

ATGAAAAAATCCCTGATTGCCCTGACTTTGGCAGCCCTTCCTGTTGCGGCAACGGCCGAT
GTTACCCTGTACGGCGCCATCAAAGCCGGCGTACAAACTTACCGTTCTGTAGAACATCGG
GAAGGCAAAGTAGTTGGCGTGGGAACCGACAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATAATCAATTTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_197_porB SEQ ID NO: 210

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAGTTGGCGTGGGAACCGACAGCGAAATCTCCGACTTCGGTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACAATAATCAATTTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_198_porB SEQ ID NO: 211

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_199_porB SEQ ID NO: 212

ATGAAAAAATCCCTGATTGCCCTGACTTTGGCAGCCCTTCCTGTTGCGGCAACGGCCGAT
GTCACCCTGTACGGCACCATCAAAGCCGGCGTACAAACTTACCGTTCTGTAGAACATACA
AAAGGCAAGGTAAGTAAAGTGGAAACCGGCAGCGAAATCGCCGACTTCGGTTCAAAAATC
GGCTTCAAAGGCCAAGAAGACCTCGGCAACGGCCTGAAGGCCGTTTGGCAGTTGGAACAA
GGTGCCTCCGTCGCCGGCACTAACACCGGCTGGGGCAACAAACAATCCTTCGTCGGCTTG
AAGGGCGGCTTCGGTACCATCCGCGCCGGTAGCCTGAACAGCCCCCTGAAAAACACCAAG
GACAACGTCAATGCTTGGGAATCCGGCAAATTTACCGGCGATGTGCTGGAAATCAGCGGA
ATGGCCAAACGGGAACACCGCTACCTGTCCGTACGCTACGATTCTCCCGAATTTGCCGGC
TTCAGCGGCAGCGTACAATACGCACCTAAAGATAATTCAGGCTCAAACGGCGAATCTTAC
CACGTTGGCTTGAACTACCGAAACGGCGGCTTCTTCGCACAATACGCCGGCTTGTTCCAA
AGATACGGCGAAGGCACTAAAAAAATCGAATACAATAATCAATTTTATAGTGTCCCCAGC
CTGTCTGTTGAAAAACTGCAAGTTCACCGTTTGGTCGGCGGTTACGACAATAATGCCCTG
TACGCCTCCGTAGCCGCACAACAACAAGATGCCAAATTGTATGGAACATGGCGTGCTAAT
TCGCACAACTCTCAAACCGAAGTTGCTGCTACCGCGGCATACCGTTTCGGCAACTTAACG
CCCCGCGTTTCTTACGCCCACGGCTTCAAAGGCTCTGTTCATAGTGCAGACTACGACAAT
ACTTATGACCAAGTGGTTGTCGGTGCGGAATACGACTTCTCCAAACGCACTTCTGCCTTG
GTTTCTGCCGGCTGGTTGCAAGCAGGCAAAGGCGCAGACAAAATCGTATCGACTGCCAGC
GCCGTCGTTCTGCGCCACAAATTCTAA

>ng_200_porB SEQ ID NO: 213

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGGAACCGGCAGCGAAARTCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCGGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_201_porB SEQ ID NO: 214

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCGGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_202_porB SEQ ID NO: 215

ATGAAAAAATCCCTGATTGCCCTGACTTTGGCAGCCCTTCCTGTTGCGGCAACGGCCGAT
GTTACCCTGTACGGCGCCATCAAAGCCGGCGTACAAACTTACCGTTCTGTAGAACATCGG
GAAGGCAAAGTAGTTGGCGTGGGAACCGGCAGCGAAATCTCCGACTTCGGTTCAAAAATC
GGCTTCAAAGGTCAAGAAGACCTCGGCAACGGCCTGAAGGCCGTTTGGCAGTTGGAACAA
GGCGCCTCCGTCGCCGGCACTAACACCGGCTGGGGCAACAAACAATCCTTCGTCGGCTTG
AAAGGCGGCTTCGGCACCATCCGCGTCGGCAGCCTGAACAGCCCCCTGAAAAACACCGGT
GCCAACGTCAATGCTTGGGAATCCGGCAAATATACCGGCGAGCTTCTGGAAATCAGCAAA
ATGGCCGGACGGGAACACCGCTACCTGTCCGCACGCTACGATTCTCCCGAATTTGCCGGC
TTCAGCGGCAGCGTACAATACGCACCTAAAGGTAATTCAGGCTCAAACGGCGAATCTTAC
CACGTTGGCTTGAACTACCGAAACAGCGGCTTCTTCGCACAATACGCCGGCTTGTTCCAA
AGATACGGCGAAGGCACTAAAAAAATCGAATACTACGATGATCAAACTTATAGTATGCCC
AGTCTGTTTGTTGAAAAACTGCAAGTTCACCGTTTGGTAGGCGGTTACGACAATAATGCC
CTGTACGTTTCCGTAGCCGCACAACAACAAGATGCCAAATTGTATGGAGCAACGAGGGTT
AATTCGCACAACTCTCAAACCGAAGTTGCCGCTACCGCGGCATACCGTTTCGGCAATGTA
ACGCCCCGCGTTTCTTACGCCCACGGCTTCAAAGGCACTGTTGATGATGCAAACCACGAC
AATACTTATGACCAAGTGGTTGTCGGTGCGGAATACGACTTCTCCAAACGCACTTCTGCC
TTGGTTTCTGCCGGCTGGTTGCAAGAAGGCAAAGGCGCAGACAAAATCGTATCGACTGCC
AGCGCCGTCGTTCTGCGCCACAAATTCTAA

>ng_203_porB SEQ ID NO: 216

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCGGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_204_porB SEQ ID NO: 217

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGAAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_205_porB SEQ ID NO: 218

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAGTTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_206_porB SEQ ID NO: 219

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCCA
GAAGGCAAAGTAATTGGCGTGGAAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_207_porB SEQ ID NO: 220

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAGTTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_208_porB SEQ ID NO: 221

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_209_porB SEQ ID NO: 222

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_210_porB SEQ ID NO: 223

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCGGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_211_porB SEQ ID NO: 224

ATGAAAAAATCCCTGATTGCCCTGACTTTGGCAGCCCTTCCTGTTGCGGCAACGGCCGAT
GTTACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCAGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_212_porB SEQ ID NO: 225

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGAAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATCGAATACGATGGTCAAACTTATAATATCCCCGGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_213_porB SEQ ID NO: 226

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGAAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGTCAAACTTATAATATCCCCGGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

>ng_214_porB SEQ ID NO: 227

ATGAAAAAATCCCTGATTGCCCTGACTTTGGCAGCCCTTCCTGTTGCGGCAACGGCCGAT
GTCACCCTGTACGGCGCCATCAAAGCCGGCGTACAAACTTACCGTTCTGTAGAACATCGG
GAAGGCAAAGTAATTGGCGTGGGAACCGGCAGCGAAATCTCCGACTTCGGTTCAAAAATC
GGCTTCAAAGGCCAAGAAGACCTCGGCAACGGCCTGAAAGCCATTTGGCAGTTGGAACAA
GGCGCCTCCGTCGCCGGCACTAACAGCGGCTGGGGCAACAAACAATCCTTCATCGGCTTG
AAGGGCGGCTTCGGCACCATCCGCGCCGGTAGCCTGAACAGCCCCCTGAAAAACACCAAG
AACAACGTCAATGCTTGGGAATCCGGCAAATTTACCGGCAATGTGCTGGAAATCAGCGGA
ATGGCCCAACGGGAACACCGCTACCTGTCCGTACGCTACGATTCTCCCGAATTTGCCGGC
TTCAGCGGCAGCGTACAATACGCACCTAAAGACAATTCAGGGTCAAACGGCGAATCTTAC
CACGTTGGCTTGAACTACCGAAACAACGGCTTCTTCGCACAATACGCCGGCTTGTTCCAA
AGATACGGCGAAGGCACTAAAAAAATGGAATACGATGGECAAACITAIAATATCCCCGGT
TTGTTTGTTGAAAAACTGCAAGTTCACCGTTTGGTCGGCGGTTACGACAATAATGCCCTG
TACGCCTCCGTAGCCGCACAACAACAAGATGCCAAATTGTATGGAGCAATGAGCGGTAAT
TCGCACAACTCTCAAACCGAAGTTGCCGCTACCGTAGCATACCGTTTCGGCAACGTAACG
CCCCGTGTTTCTTACGCCCACGGCTTCAAAGGCACTGTTGATGATGCAAACCACGACAAT
ACTTACGACCAAGTGGTTGTCGGTGCGGAATACGACTTCTCCAAACGCACTTCTGCCTTG
GTTTCTGCCGGCTGGTTGCAAGAAGGCAAAGGCGCAGACAAAATCGTATCGACTGCCAGC
GCCGTCGTTCTGCGCCACAAATTCTAA

### ANNEX E

### Sequences for Exemplary RNA With Low C:T Ratios to Be Used for Normalization

### 1. NGO0066a: NC_002946.2:c69710-69021

DNA (- strand): SEQ ID NO: 231

GTGCAGGCGGATTTAGCCTACGCCGCCGAACGCATTACCCACGATTATCCGGAA
CCAACCGGTGCAAAAAAAGGCAAAATAAGCACGGTAAGCGATTATTTCAGAAAC
ATCCGTACGCATTCCATCCACCCCAGGGTGTCGGTCGGCTACGACTTCGGCGGCT
GGAGGATAGCGGCAGATTATGCCCGTTACAGAAAGTGGAACAACAGTAAATATT
CCGTCAACACAAAAAAGGTGAACGAAAACAAGGGCGAAAAGATAAACGTGACG
CAATATCTGAAGGCGGAAAATCAGGAAAACGGTACGTTCCACGCCGTTTCTTCTC
TCGGCTTGTCCGCCGTTTACGATTTCAAACTCAACGACAAATTCAAACCCTATAT
CGGCATGCGCGTCGGCTACGGGCACGTCAGACATCAGGTTCGTTCGGTTGAACA
AGAAACCACGACTGTTACCACTTACCTACAGAGTGGTAAGCCAAGTCCTATCGT
ACGAGGTTCGACCCTCAAACTTCCCCATCACGAAAGCCGCAGCAGCCGCCGCTT
GGGCTTCGGCGCGATGGCGGGCGTGGGCATAGACGTCGCGCCCGGTCTGACCTT
GGACGCCGGCTACCGCTACCACTATTGGGGACGCCTGGAAAACACCCGCTTCAA
AACCCACGAAGCCTCGTTGGGCGTGCGCTACCGCTTCTGA

RNA SEQ ID NO: 232

GUGCAGGCGGAUUUAGCCUACGCCGCCGAACGCAUUACCCACGAUUAUCCGGA
ACCAACCGGUGCAAAAAAAGGCAAAAUAAGCACGGUAAGCGAUUAUUUCAGA
AACAUCCGUACGCAUUCCAUCCACCCCAGGGUGUCGGUCGGCUACGACUUCGG
CGGCUGGAGGAUAGCGGCAGAUUAUGCCCGUUACAGAAAGUGGAACAACAGU
AAAUAUUCCGUCAACACAAAAAAGGUGAACGAAAACAAGGGCGAAAAGAUAA
ACGUGACGCAAUAUCUGAAGGCGGAAAAUCAGGAAAACGGUACGUUCCACGC
CGUUUCUUCUCUCGGCUUGUCCGCCGUUUACGAUUUCAAACUCAACGACAAAU
UCAAACCCUAUAUCGGCAUGCGCGUCGGCUACGGGCACGUCAGACAUCAGGUU
CGUUCGGUUGAACAAGAAACCACGACUGUUACCACUUACCUACAGAGUGGUA
AGCCAAGUCCUAUCGUACGAGGUUCGACCCUCAAACUUCCCCAUCACGAAAGC
CGCAGCAGCCGCCGCUUGGGCUUCGGCGCGAUGGCGGGCGUGGGCAUAGACGU
CGCGCCCGGUCUGACCUUGGACGCCGGCUACCGCUACCACUAUUGGGGACGCC
UGGAAAACACCCGCUUCAAAACCCACGAAGCCUCGUUGGGCGUGCGCUACCGC
UUCUGA

cDNA: SEQ ID NO: 233

TCAGAAGCGGTAGCGCACGCCCAACGAGGCTTCGTGGGTTTTGAAGCGGGTGTT
TTCCAGGCGTCCCCAATAGTGGTAGCGGTAGCCGGCGTCCAAGGTCAGACCGGG
CGCGACGTCTATGCCCACGCCCGCCATCGCGCCGAAGCCCAAGCGGCGGCTGCT
GCGGCTTTCGTGATGGGGAAGTTTGAGGGTCGAACCTCGTACGATAGGACTTGG
CTTACCACTCTGTAGGTAAGTGGTAACAGTCGTGGTTTCTTGTTCAACCGAACGA
ACCTGATGTCTGACGTGCCCGTAGCCGACGCGCATGCCGATATAGGGTTTGAATT
TGTCGTTGAGTTTGAAATCGTAAACGGCGGACAAGCCGAGAGAAGAAACGGCGT
GGAACGTACCGTTTTCCTGATTTTCCGCCTTCAGATATTGCGTCACGTTTATCTTT
TCGCCCTTGTTTTCGTTCACCTTTTTTGTGTTGACGGAATATTTACTGTTGTTCCAC
TTTCTGTAACGGGCATAATCTGCCGCTATCCTCCAGCCGCCGAAGTCGTAGCCGA
CCGACACCCTGGGGTGGATGGAATGCGTACGGATGTTTCTGAAATAATCGCTTAC
CGTGCTTATTTTGCCTTTTTTTGCACCGGTTGGTTCCGGATAATCGTGGGTAATGC
GTTCGGCGGCGTAGGCTAAATCCGCCTGCAC

### 2. NGO0070: NC_002946.2: C75580-74783

DNA (- strand): SEQ ID NO: 234

ATGAATCCAGCCCGCAAAAAACCTTCTCTTCTCTTCTCTTCTCTTCTCTTCTCTTCT
CTTCTCTTCTCTTCGGCAGCGCAGGCGGCAAGTGAAGGCAATGGCCGCGGCCCG
TATGTGCAGGCGGATTTAGCCTACGCCGCCGAACGCATTACCCACGATTATCCGG
AACCAACCGCTCCAGGCAAAAACAAAATAAGCACGGTAAGCGATTATTTCAGAA
ACATCCGTACGCATTCCATCCACCCCAGGGTGTCGGTCGGCTACGACTTCGGCGG
CTGGAGGATAGCGGCAGATTATGCCCGTTACAGAAAGTGGAACGACAATAAATA
TTCCGTCGACATAAAAGAGTTGGAAAACAAGAATCAGAATAAGAGAGACCTGA
AGACGGAAAATCAGGAAAACGGCAGCTTCCACGCCGTTTCTTCTCTCGGCTTATC
AGCCGTTTACGATTTCAAACTCAACGACAAATTCAAACCCTATATCGGTGCGCGC
GTCGCCTACGGACACGTCAGACACAGCATCGATTCGACTAAAAAAATAACAGGT
ACTCTTACCGCCTACCCTAGTGATGCTGACGCAGCAGTTACGGTTTATCCTGACG
GACATCCGCAAAAAAACACCTATCAAAAAAGCAACAGCAGCCGCCGCTTGGGCT
TCGGCGCGATGGCGGGCGTGGGCATAGACGTCGCGCCCGGCCTGACCTTGGACG
CCGGCTACCGCTACCACAACTGGGGACGCTTGGAAAACACCCGCTTCAAAACCC
ACGAAGCCTCATTGGGCATGCGCTACCGCTTCTGA

RNA: SEQ ID NO: 235

AUGAAUCCAGCCCGCAAAAAACCUUCUCUUCUCUUCUCUUCUCUUCUCUUCUC
UUCUCUUCUCUUCUCUUCGGCAGCGCAGGCGGCAAGUGAAGGCAAUGGCCGCG
GCCCGUAUGUGCAGGCGGAUUUAGCCUACGCCGCCGAACGCAUUACCCACGAU
UAUCCGGAACCAACCGCUCCAGGCAAAAACAAAAUAAGCACGGUAAGCGAUU
AUUUCAGAAACAUCCGUACGCAUUCCAUCCACCCCAGGGUGUCGGUCGGCUAC
GACUUCGGCGGCUGGAGGAUAGCGGCAGAUUAUGCCCGUUACAGAAAGUGGA
ACGACAAUAAAUAUUCCGUCGACAUAAAAGAGUUGGAAAACAAGAAUCAGAA
UAAGAGAGACCUGAAGACGGAAAAUCAGGAAAACGGCAGCUUCCACGCCGUU
UCUUCUCUCGGCUUAUCAGCCGUUUACGAUUUCAAACUCAACGACAAAUUCAA
ACCCUAUAUCGGUGCGCGCGUCGCCUACGGACACGUCAGACACAGCAUCGAUU
CGACUAAAAAAAUAACAGGUACUCUUACCGCCUACCCUAGUGAUGCUGACGCA
GCAGUUACGGUUUAUCCUGACGGACAUCCGCAAAAAAACACCUAUCAAAAAA
GCAACAGCAGCCGCCGCUUGGGCUUCGGCGCGAUGGCGGGCGUGGGCAUAGAC
GUCGCGCCCGGCCUGACCUUGGACGCCGGCUACCGCUACCACAACUGGGGACG
CUUGGAAAACACCCGCUUCAAAACCCACGAAGCCUCAUUGGGCAUGCGCUACC
GCUUCUGA

cDNA: SEQ ID NO: 236

TCAGAAGCGGTAGCGCATGCCCAATGAGGCTTCGTGGGTTTTGAAGCGGGTGTTT
TCCAAGCGTCCCCAGTTGTGGTAGCGGTAGCCGGCGTCCAAGGTCAGGCCGGGC
GCGACGTCTATGCCCACGCCCGCCATCGCGCCGAAGCCCAAGCGGCGGCTGCTG
TTGCTTTTTTGATAGGTGTTTTTTTGCGGATGTCCGTCAGGATAAACCGTAACTGC
TGCGTCAGCATCACTAGGGTAGGCGGTAAGAGTACCTGTTATTTTTTTAGTCGAA
TCGATGCTGTGTCTGACGTGTCCGTAGGCGACGCGCGCACCGATATAGGGTTTGA
ATTTGTCGTTGAGTTTGAAATCGTAAACGGCTGATAAGCCGAGAGAAGAAACGG
CGTGGAAGCTGCCGTTTTCCTGATTTTCCGTCTTCAGGTCTCTCTTATTCTGATTC
TTGTTTTCCAACTCTTTTATGTCGACGGAATATTTATTGTCGTTCCACTTTCTGTA
ACGGGCATAATCTGCCGCTATCCTCCAGCCGCCGAAGTCGTAGCCGACCGACAC
CCTGGGGTGGATGGAATGCGTACGGATGTTTCTGAAATAATCGCTTACCGTGCTT
ATTTTGTTTTTGCCTGGAGCGGTTGGTTCCGGATAATCGTGGGTAATGCGTTCGG
CGGCGTAGGCTAAATCCGCCTGCACATACGGGCCGCGGCCATTGCCTTCACTTGC
CGCCTGCGCTGCCGAAGAGAAGAGAAGAGAAGAGAAGAGAAGAGAAGAGAAG
AGAAGAGAAGGTTTTTTGCGGGCTGGATTCAT

### 3. Ngo0372: Nc_002946.2:366358-367185

DNA (+ strand): SEQ ID NO: 237

ATGATGTTGAAAAAATTCGTACTCGGCGGCATTGCCGCATTGGTTTTGGCGGCCT
GCGGCGGTTCGGAAGGCGGCAGCGGAGCATCTTCCGCGCCTGCACAATCGGCAA
TTTCCGGTTCTTTAATCGAGCGCATCAACAATAAAGGCACGGTTACCGTCGGCAC
GGAAGGCACTTACGCACCGTTTACCTACCACGACAAAGACGGCAAACTGACCGG
TTACGACGTGGAAGTAACCCGCGCCGTGGCGGAAAAACTGGGCGTAAAAGTCGA
GTTTAAAGAAACGCAATGGGATTCGATGATGGCGGGTTTGAAAGCCGGACGTTT
CGACGTGGTGGCAAACCAAGTCGGCCTGACCAGCCCCGAACGCCAGGCGACATT
TGACAAATCCGAACCTTACAGCTGGAGCGGTGCGGTTTTGGTTGCGCATAACGA
CAGCAACATTAAATCCATAGCCGACATCAAAGGCGTGAAAACCGCGCAATCCCT
GACCAGCAACTACGGCGAAAAAGCCAAAGCCGCAGGTGCGCAACTCGTGCCGG
TGGACGGTTTGGCGCAATCGCTGACCCTGATTGAACAAAAACGCGCCGATGCGA
CGTTGAACGATGAATTGGCGGTTTTGGACTATCTGAAGAAAAACCCGAATGCGG
GGGTGAAAATCGTGTGGTCCGCGCCTGCCGATGAAAAAGTCGGTTCCGGTCTGA
TTGTCAACAAGGGCAATGACGAGGCCGTGGCGAAATTCAGCACGGCAATCAACG
AGCTGAAAGCCGACGGCACGTTGAAAAAACTGGGCGAACAATTCTTCGGAAAAG
ACATCAGTGTTCAATAA

RNA SEQ ID NO: 238

AUGAUGUUGAAAAAAUUCGUACUCGGCGGCAUUGCCGCAUUGGUUUUGGCGG
CCUGCGGCGGUUCGGAAGGCGGCAGCGGAGCAUCUUCCGCGCCUGCACAAUCG
GCAAUUUCCGGUUCUUUAAUCGAGCGCAUCAACAAUAAAGGCACGGUUACCG
UCGGCACGGAAGGCACUUACGCACCGUUUACCUACCACGACAAAGACGGCAAA
CUGACCGGUUACGACGUGGAAGUAACCCGCGCCGUGGCGGAAAAACUGGGCG
UAAAAGUCGAGUUUAAAGAAACGCAAUGGGAUUCGAUGAUGGCGGGUUUGAA
AGCCGGACGUUUCGACGUGGUGGCAAACCAAGUCGGCCUGACCAGCCCCGAAC
GCCAGGCGACAUUUGACAAAUCCGAACCUUACAGCUGGAGCGGUGCGGUUUU
GGUUGCGCAUAACGACAGCAACAUUAAAUCCAUAGCCGACAUCAAAGGCGUG
AAAACCGCGCAAUCCCUGACCAGCAACUACGGCGAAAAAGCCAAAGCCGCAGG
UGCGCAACUCGUGCCGGUGGACGGUUUGGCGCAAUCGCUGACCCUGAUUGAAC
AAAAACGCGCCGAUGCGACGUUGAACGAUGAAUUGGCGGUUUUGGACUAUCU
GAAGAAAAACCCGAAUGCGGGGGUGAAAAUCGUGUGGUCCGCGCCUGCCGAU
GAAAAAGUCGGUUCCGGUCUGAUUGUCAACAAGGGCAAUGACGAGGCCGUGG
CGAAAUUCAGCACGGCAAUCAACGAGCUGAAAGCCGACGGCACGUUGAAAAA
ACUGGGCGAACAAUUCUUCGGAAAAGACAUCAGUGUUCAAUAA

cDNA: SEQ ID NO: 239

TTATTGAACACTGATGTCTTTTCCGAAGAATTGTTCGCCCAGTTTTTTCAACGTGC
CGTCGGCTTTCAGCTCGTTGATTGCCGTGCTGAATTTCGCCACGGCCTCGTCATTG
CCCTTGTTGACAATCAGACCGGAACCGACTTTTTCATCGGCAGGCGCGGACCAC
ACGATTTTCACCCCCGCATTCGGGTTTTTCTTCAGATAGTCCAAAACCGCCAATT
CATCGTTCAACGTCGCATCGGCGCGTTTTTGTTCAATCAGGGTCAGCGATTGCGC
CAAACCGTCCACCGGCACGAGTTGCGCACCTGCGGCTTTGGCTTTTTCGCCGTAG
TTGCTGGTCAGGGATTGCGCGGTTTTCACGCCTTTGATGTCGGCTATGGATTTAA
TGTTGCTGTCGTTATGCGCAACCAAAACCGCACCGCTCCAGCTGTAAGGTTCGGA
TTTGTCAAATGTCGCCTGGCGTTCGGGGCTGGTCAGGCCGACTTGGTTTGCCACC
ACGTCGAAACGTCCGGCTTTCAAACCCGCCATCATCGAATCCCATTGCGTTTCTT
TAAACTCGACTTTTACGCCCAGTTTTTCCGCCACGGCGCGGGTTACTTCCACGTC
GTAACCGGTCAGTTTGCCGTCTTTGTCGTGGTAGGTAAACGGTGCGTAAGTGCCT
TCCGTGCCGACGGTAACCGTGCCTTTATTGTTGATGCGCTCGATTAAAGAACCGG
AAATTGCCGATTGTGCAGGCGCGGAAGATGCTCCGCTGCCGCCTTCCGAACCGC
CGCAGGCCGCCAAAACCAATGCGGCAATGCCGCCGAGTACGAATTTTTTCAACA
TCAT

### 4. Ngo0374: Nc_002946.2:367901-368656

DNA (+ strand): SEQ ID NO: 240

ATGATTAAAATCCGCAATATCCATAAGACCTTTGGCGAAAACACCATTTTGCGCG
GCATCGATTTGGATGTGGGCAAAGGGCAGGTGGTCGTCATCCTCGGGCCTTCCG
GCTCGGGTAAAACAACATTTCTGCGCTGCCTAAACGCGTTGGAAATGCCCGAAG
ACGGACAAATCGAGTTCGACAACGCGCGGCCGTTACGCATTGATTTTTCCAAAA
AAACAAGCAAACACGATATTTTGGCACTGCGCCGCAAGTCCGGAATGGTATTCC
AACAATACAACCTCTTCCCGCATAAAACCGTGTTGGAAAACGTGATGGAAGGGC
CGGTTGCCGTACAGGGCAAGCCTGCCGCCCAAGCGCGCGAAGAGGCTTTGAAAC
TGCTGGAAAAAGTCGGCTTGGGCGATAAAGTGGATTTGTATCCCTACCAGCTTTC
CGGCGGTCAGCAGCAGCGTGTCGGTATCGCCCGCGCACTGGCGATTCAGCCTGA
ATTGATGCTGTTTGACGAACCCACTTCCGCGCTGGACCCCGAGTTGGTGCAAGAC
GTGTTGGACGCCATGAAGGAATTGGCGCGGGAAGGTTGGACGATGGTCGTCGTT
ACCCACGAAATCAAGTTCACGCTGGAAGTTGCCACGAACGTCGTCGTGATGGAC
GGCGGCGTTATCGTAGAGCAGGGCAGCCCGAAAGAGTTGTTCGACCACCTCAAA
CACGAACGGACGCGGAGATTTTTAAGCCAAATCCAATCTGCCAAGATTTGA

RNA SEQ ID NO: 241

AUGAUUAAAAUCCGCAAUAUCCAUAAGACCUUUGGCGAAAACACCAUUUUGC
GCGGCAUCGAUUUGGAUGUGGGCAAAGGGCAGGUGGUCGUCAUCCUCGGGCC
UUCCGGCUCGGGUAAAACAACAUUUCUGCGCUGCCUAAACGCGUUGGAAAUG
CCCGAAGACGGACAAAUCGAGUUCGACAACGCGCGGCCGUUACGCAUUGAUUU
UUCCAAAAAAACAAGCAAACACGAUAUUUUGGCACUGCGCCGCAAGUCCGGA
AUGGUAUUCCAACAAUACAACCUCUUCCCGCAUAAAACCGUGUUGGAAAACG
UGAUGGAAGGGCCGGUUGCCGUACAGGGCAAGCCUGCCGCCCAAGCGCGCGAA
GAGGCUUUGAAACUGCUGGAAAAAGUCGGCUUGGGCGAUAAAGUGGAUUUGU
AUCCCUACCAGCUUUCCGGCGGUCAGCAGCAGCGUGUCGGUAUCGCCCGCGCA
CUGGCGAUUCAGCCUGAAUUGAUGCUGUUUGACGAACCCACUUCCGCGCUGGA
CCCCGAGUUGGUGCAAGACGUGUUGGACGCCAUGAAGGAAUUGGCGCGGGAA
GGUUGGACGAUGGUCGUCGUUACCCACGAAAUCAAGUUCACGCUGGAAGUUG
CCACGAACGUCGUCGUGAUGGACGGCGGCGUUAUCGUAGAGCAGGGCAGCCCG
AAAGAGUUGUUCGACCACCUCAAACACGAACGGACGCGGAGAUUUUUAAGCC
AAAUCCAAUCUGCCAAGAUUUGA

cDNA: SEQ ID NO: 242

TCAAATCTTGGCAGATTGGATTTGGCTTAAAAATCTCCGCGTCCGTTCGTGTTTG
AGGTGGTCGAACAACTCTTTCGGGCTGCCCTGCTCTACGATAACGCCGCCGTCCA
TCACGACGACGTTCGTGGCAACTTCCAGCGTGAACTTGATTTCGTGGGTAACGAC
GACCATCGTCCAACCTTCCCGCGCCAATTCCTTCATGGCGTCCAACACGTCTTGC
ACCAACTCGGGGTCCAGCGCGGAAGTGGGTTCGTCAAACAGCATCAATTCAGGC
TGAATCGCCAGTGCGCGGGCGATACCGACACGCTGCTGCTGACCGCCGGAAAGC
TGGTAGGGATACAAATCCACTTTATCGCCCAAGCCGACTTTTTCCAGCAGTTTCA
AAGCCTCTTCGCGCGCTTGGGCGGCAGGCTTGCCCTGTACGGCAACCGGCCCTTC
CATCACGTTTTCCAACACGGTTTTATGCGGGAAGAGGTTGTATTGTTGGAATACC
ATTCCGGACTTGCGGCGCAGTGCCAAAATATCGTGTTTGCTTGTTTTTTTGGAAA
AATCAATGCGTAACGGCCGCGCGTTGTCGAACTCGATTTGTCCGTCTTCGGGCAT
TTCCAACGCGTTTAGGCAGCGCAGAAATGTTGTTTTACCCGAGCCGGAAGGCCC
GAGGATGACGACCACCTGCCCTTTGCCCACATCCAAATCGATGCCGCGCAAAAT
GGTGTTTTCGCCAAAGGTCTTATGGATATTGCGGATTTTAATCAT

### 5. NGO0399: NC_002946.2: C392291-391452

DNA (- strand): SEQ ID NO: 243

GTGAAACGCATTTTTCTGTTTTTGGCTACCAATATCGCTGTTTTGGTCGTAATCAA
CATTGTTTTGGCGGTTCTGGGCATCAACAGCCGGGGCGGCGCGGGCAGCCTGTTG
GCGTATTCCGCCGTCGTCGGCTTCACTGGTTCGATTATTTCGCTGCTGATGTCCAA
ATTTATCGCCAAACAATCGGTCGGTGCGGAAGTCATCGACACGCCGCGCACCGA
AGAAGAAGCCTGGCTTCTGAACACTGTCGAAGCCCAAGCGCGGCAATGGAATCT
GAAAACGCCAGAAGTCGCCATCTACCACTCCCCCGAACCCAATGCCTTTGCCAC
GGGCGCATCGAGAAACAGCTCCCTGATCGCCGTCAGCACCGGTTTGCTCGACCA
TATGACGCGCGACGAAGTGGAAGCCGTGTTGGCGCACGAAATGGCGCACGTCGG
CAACGGCGACATGGTTACGCTGACGCTGATTCAAGGCGTGGTCAATACCTTTGTC
GTGTTCCTGTCGCGCATTATTGCCAACCTGATTGCCCGAAACAACGACGGCAGCC
AGTCCCAGGGAACTTATTTCCTAGTCAGCATGGTATTCCAAATCCTGTTCGGCTT
CCTTGCCAGCCTGATTGTCATGTGGTTCAGCCGCCAACGCGAATACCGCGCCGAC
GCGGGCGCGGCAAAACTGGTCGGCGCACCGAAAATGATTTCCGCCCTGCAAAGG
CTTAAAGGCAACCCGGTCGATTTGCCCGAAGAAATGAACGCAATGGGCATCGCC
GGAGATACGCGCGACTCCCTGCTCAGCACCCACCCTTCGCTGGACAACCGAATC
GCCCGCCTCAAATCGCTTTAA

RNA SEQ ID NO: 244

GUGAAACGCAUUUUUCUGUUUUUGGCUACCAAUAUCGCUGUUUUGGUCGUAA
UCAACAUUGUUUUGGCGGUUCUGGGCAUCAACAGCCGGGGCGGCGCGGGCAG
CCUGUUGGCGUAUUCCGCCGUCGUCGGCUUCACUGGUUCGAUUAUUUCGCUGC
UGAUGUCCAAAUUUAUCGCCAAACAAUCGGUCGGUGCGGAAGUCAUCGACAC
GCCGCGCACCGAAGAAGAAGCCUGGCUUCUGAACACUGUCGAAGCCCAAGCGC
GGCAAUGGAAUCUGAAAACGCCAGAAGUCGCCAUCUACCACUCCCCCGAACCC
AAUGCCUUUGCCACGGGCGCAUCGAGAAACAGCUCCCUGAUCGCCGUCAGCAC
CGGUUUGCUCGACCAUAUGACGCGCGACGAAGUGGAAGCCGUGUUGGCGCAC
GAAAUGGCGCACGUCGGCAACGGCGACAUGGUUACGCUGACGCUGAUUCAAG
GCGUGGUCAAUACCUUUGUCGUGUUCCUGUCGCGCAUUAUUGCCAACCUGAU
UGCCCGAAACAACGACGGCAGCCAGUCCCAGGGAACUUAUUUCCUAGUCAGCA
UGGUAUUCCAAAUCCUGUUCGGCUUCCUUGCCAGCCUGAUUGUCAUGUGGUU
CAGCCGCCAACGCGAAUACCGCGCCGACGCGGGCGCGGCAAAACUGGUCGGCG
CACCGAAAAUGAUUUCCGCCCUGCAAAGGCUUAAAGGCAACCCGGUCGAUUUG
CCCGAAGAAAUGAACGCAAUGGGCAUCGCCGGAGAUACGCGCGACUCCCUGCU
CAGCACCCACCCUUCGCUGGACAACCGAAUCGCCCGCCUCAAAUCGCUUUAA

cDNA: SEQ ID NO: 245

TTAAAGCGATTTGAGGCGGGCGATTCGGTTGTCCAGCGAAGGGTGGGTGCTGAG
CAGGGAGTCGCGCGTATCTCCGGCGATGCCCATTGCGTTCATTTCTTCGGGCAAA
TCGACCGGGTTGCCTTTAAGCCTTTGCAGGGCGGAAATCATTTTCGGTGCGCCGA
CCAGTTTTGCCGCGCCCGCGTCGGCGCGGTATTCGCGTTGGCGGCTGAACCACAT
GACAATCAGGCTGGCAAGGAAGCCGAACAGGATTTGGAATACCATGCTGACTAG
GAAATAAGTTCCCTGGGACTGGCTGCCGTCGTTGTTTCGGGCAATCAGGTTGGCA
ATAATGCGCGACAGGAACACGACAAAGGTATTGACCACGCCTTGAATCAGCGTC
AGCGTAACCATGTCGCCGTTGCCGACGTGCGCCATTTCGTGCGCCAACACGGCTT
CCACTTCGTCGCGCGTCATATGGTCGAGCAAACCGGTGCTGACGGCGATCAGGG
AGCTGTTTCTCGATGCGCCCGTGGCAAAGGCATTGGGTTCGGGGGAGTGGTAGA
TGGCGACTTCTGGCGTTTTCAGATTCCATTGCCGCGCTTGGGCTTCGACAGTGTTC
AGAAGCCAGGCTTCTTCTTCGGTGCGCGGCGTGTCGATGACTTCCGCACCGACCG
ATTGTTTGGCGATAAATTTGGACATCAGCAGCGAAATAATCGAACCAGTGAAGC
CGACGACGGCGGAATACGCCAACAGGCTGCCCGCGCCGCCCCGGCTGTTGATGC
CCAGAACCGCCAAAACAATGTTGATTACGACCAAAACAGCGATATTGGTAGCCA
AAAACAGAAAAATGCGTTTCAC

### 6. Ngo0453: Nc_002946.2:447935-448546

DNA (+ strand): SEQ ID NO: 246

ATGAAGAATAATGATTGCTTGCGCCTGAAAAATCCCCAGTCCGGTATGGCGTTG
ATAGAAGTCTTGGTCGCTATGCTCGTTCTGACCATCGGTATTTTGGCATTGCTGTC
CGTACAGTTGCGGACAGTCGCTTCCGTCAGGGAGGCGGAAACGCAAACCATCGT
CAGCCAAATCACGCAAAACCTGATGGAAGGAATGTTGATGAATCCGACCATTGA
TTTGGACAGCAACAAGAAAAACTATAGTCTTTACATGGGAAAACAGACACTATC
AGCTGTGGATGGTGAGTTTATGCTTGATGCCGAGAAAAGTAAGGCGCAGTTGGC
AGAGGAACAATTGAAGAGATTTAGTCATGAGCTGAAAAATGCCTTGCCGGATGC
GGTAGCTATTCATTACGCCGTCTGCAAGGATTCGTCGGGTGACGCGCCGACATTG
TCCGACAGCGGTGCTTTTTCTTCAAATTGCGACAATAAGGCAAACGGGGATACTT
TGATTAAAGTATTGTGGGTAAATGATTCGGCAGGGGATTCGGATATTTCCCGTAC
GAATCTTGAAGTGAGCGGCGACAATATCGTATATACCTATCAGGCAAGGGTCGG
AGGTCGTGAATGA

RNA SEQ ID NO: 247

AUGAAGAAUAAUGAUUGCUUGCGCCUGAAAAAUCCCCAGUCCGGUAUGGCGU
UGAUAGAAGUCUUGGUCGCUAUGCUCGUUCUGACCAUCGGUAUUUUGGCAUU
GCUGUCCGUACAGUUGCGGACAGUCGCUUCCGUCAGGGAGGCGGAAACGCAA
ACCAUCGUCAGCCAAAUCACGCAAAACCUGAUGGAAGGAAUGUUGAUGAAUC
CGACCAUUGAUUUGGACAGCAACAAGAAAAACUAUAGUCUUUACAUGGGAAA
ACAGACACUAUCAGCUGUGGAUGGUGAGUUUAUGCUUGAUGCCGAGAAAAGU
AAGGCGCAGUUGGCAGAGGAACAAUUGAAGAGAUUUAGUCAUGAGCUGAAAA
AUGCCUUGCCGGAUGCGGUAGCUAUUCAUUACGCCGUCUGCAAGGAUUCGUC
GGGUGACGCGCCGACAUUGUCCGACAGCGGUGCUUUUUCUUCAAAUUGCGAC
AAUAAGGCAAACGGGGAUACUUUGAUUAAAGUAUUGUGGGUAAAUGAUUCGG
CAGGGGAUUCGGAUAUUUCCCGUACGAAUCUUGAAGUGAGCGGCGACAAUAU
CGUAUAUACCUAUCAGGCAAGGGUCGGAGGUCGUGAAUGA

cDNA: SEQ ID NO: 248

TCATTCACGACCTCCGACCCTTGCCTGATAGGTATATACGATATTGTCGCCGCTC
ACTTCAAGATTCGTACGGGAAATATCCGAATCCCCTGCCGAATCATTTACCCACA
ATACTTTAATCAAAGTATCCCCGTTTGCCTTATTGTCGCAATTTGAAGAAAAAGC
ACCGCTGTCGGACAATGTCGGCGCGTCACCCGACGAATCCTTGCAGACGGCGTA
ATGAATAGCTACCGCATCCGGCAAGGCATTTTTCAGCTCATGACTAAATCTCTTC
AATTGTTCCTCTGCCAACTGCGCCTTACTTTTCTCGGCATCAAGCATAAACTCAC
CATCCACAGCTGATAGTGTCTGTTTTCCCATGTAAAGACTATAGTTTTTCTTGTTG
CTGTCCAAATCAATGGTCGGATTCATCAACATTCCTTCCATCAGGTTTTGCGTGA
TTTGGCTGACGATGGTTTGCGTTTCCGCCTCCCTGACGGAAGCGACTGTCCGCAA
CTGTACGGACAGCAATGCCAAAATACCGATGGTCAGAACGAGCATAGCGACCAA
GACTTCTATCAACGCCATACCGGACTGGGGATTTTTCAGGCGCAAGCAATCATTA
TTCTTCAT

### 7. Ng00571: Nc_002946.2:553869-555665

DNA (+ strand): SEQ ID NO: 249

ATGCGCTACAAACCCCTTCTGCTTGCCCTGATGCTCGTTTTTTCCACGCCCGCCGT
TGCCGCCCACGACGCGGCACACAACCGTTCCGCCGAAGTGAAAAAACAGGCGA
AGAACAAAAAAGAACAGCCCGAAGCGGCGGAAGGCAAAAAAGAAAAAGGCAA
AAATGCCGCAGTGAAAGATAAAAAAACAGGCGGCAAAGAGGCGGCAAAAGAGT
TCAAAAAAACCGCCAAAAACCGCAAAGAAGCAGAGAAGGAGGCGACATCCAGG
CAGTCTGCGCGCAAAGGACGCGAAGGGGATAAGGAATCGAAGGCGGAACACAA
AAAGGCACATGGCAAGCCCGTGTCCGGATCCAAAGAAAAAAACGCAAAAACAC
AGCCTGAAAACAAACAAGGCAAAAAAGGGGCAAAAGGACAGGGCAATCCGCGC
AAGGGCGGCAAGGCGGAAAAAGACACTGTTTCTGCAAATAAAAAAGCCCGTTCC
GACAAGAACGGCAAAGCAGTGAAACAGGACAAAAAACACACGGAAGAGAAAA
ATGCCAAAACCGATTCCGACGAATTGAAAGCCGCCGTTGCCGCTGCCACCAATG
ATGTCGAAAACAAAAAAGCCCTGCTCAAACAAAGCGAAGGAATGCTGCTTCATG
TCAGCAATTCCCTCAAACAGCTTCAGGAAGAGCGTATCCGCCAAGAACGTATCC
GCCAAGAGCGTATCCGTCAGGCGCGCGGCAACCTTGCTTCCGTCAACCGCAAAC
AGCGCGAGGCTTGGGACAAATTCCAAAAACTCAATACCGAGCTGAACCGTTTGA
AAACGGAAGTCGCCGCTACGAAAGCGCAGATTTCCCGTTTCGTATCGGGGAACT
ATAAAAACAGCCGGCCGAATGCGGTTGCCCTGTTCCTGAAAAACGCCGAACCGG
GTCAGAAAAACCGCTTTTTGCGTTATACGCGTTATGTAAACGCCTCCAATCGGGA
AGTTGTCAAGGATTTGGAAAAACAGCAGAAGGCTTTGGCGGTACAAGAGCAGAA
AATCAACAATGAGCTTGCCCGTTTGAAGAAAATTCAGGCAAACGTGCAATCCCT
GCTGAAAAAACAGGGTGTAACCGATGCGGCGGAACAGACGGAAAGCCGCAGAC
AGAATGCCAAAATCTCCAAAGATGCCCGAAAACTGCTGGAACAGAAAGGGAAC
GAGCAGCAGCTGAACAAGCTCTTGAGCAATTTGGAGAAAAAAAAAGCCGAACA
CCGCATTCAGGATGCGGAAGCAAAAAGAAAATTGGCTGAAGCCAAACTGGCGG
CAGCCGAAAAAGCCAGAAAAGAAGCGGCGCAGCAGAAGGCTGAAGCGCGACGT
GCGGAAATGTCCAACCTGACCGCCGAAGACAGGAACATCCAAGCGCCTTCGGTT
ATGGGTATCGGCAGTGCCGACGGTTTCAGCCGCATGCAGGGACGTTTGAAAAAA
CCGGTTGACGGTGTGCCGACCGGGCTTTTCGGGCAGAACCGGAGCGGCGGCGAT
GTTTGGAAAGGCGTGTTCTATTCCACTGCGCCTGCAACGGTTGAAAGCATTGCGC
CGGGAACGGTAAGCTATGCGGACGAGTTGGACGGCTACGGCAAAGTGGTCGTGA
TCGATCACGGCGAGAACTACATCAGCATCTATGCCGGTTTGAGCGAAATTTCCGC
CGGCAAGGGTTATACGGTCGCGGCAGGAAGCAAAATCGGCACGAGCGGGTCGC
TGCCGGACGGGGAAGAGGGGCTTTACCTGCAAATACGTTATCGAGGTCAGGTGT
TGAACCCTTCGGGCTGGATACGTTGA

RNA: SEQ ID NO: 250

AUGCGCUACAAACCCCUUCUGCUUGCCCUGAUGCUCGUUUUUUCCACGCCCGC
CGUUGCCGCCCACGACGCGGCACACAACCGUUCCGCCGAAGUGAAAAAACAGG
CGAAGAACAAAAAAGAACAGCCCGAAGCGGCGGAAGGCAAAAAAGAAAAAGG
CAAAAAUGCCGCAGUGAAAGAUAAAAAAACAGGCGGCAAAGAGGCGGCAAAA
GAGUUCAAAAAAACCGCCAAAAACCGCAAAGAAGCAGAGAAGGAGGCGACAU
CCAGGCAGUCUGCGCGCAAAGGACGCGAAGGGGAUAAGGAAUCGAAGGCGGA
ACACAAAAAGGCACAUGGCAAGCCCGUGUCCGGAUCCAAAGAAAAAAACGCA
AAAACACAGCCUGAAAACAAACAAGGCAAAAAAGGGGCAAAAGGACAGGGCA
AUCCGCGCAAGGGCGGCAAGGCGGAAAAAGACACUGUUUCUGCAAAUAAAAA
AGCCCGUUCCGACAAGAACGGCAAAGCAGUGAAACAGGACAAAAAACACACG
GAAGAGAAAAAUGCCAAAACCGAUUCCGACGAAUUGAAAGCCGCCGUUGCCG
CUGCCACCAAUGAUGUCGAAAACAAAAAAGCCCUGCUCAAACAAAGCGAAGG
AAUGCUGCUUCAUGUCAGCAAUUCCCUCAAACAGCUUCAGGAAGAGCGUAUCC
GCCAAGAACGUAUCCGCCAAGAGCGUAUCCGUCAGGCGCGCGGCAACCUUGCU
UCCGUCAACCGCAAACAGCGCGAGGCUUGGGACAAAUUCCAAAAACUCAAUAC
CGAGCUGAACCGUUUGAAAACGGAAGUCGCCGCUACGAAAGCGCAGAUUUCCC
GUUUCGUAUCGGGGAACUAUAAAAACAGCCGGCCGAAUGCGGUUGCCCUGUU
CCUGAAAAACGCCGAACCGGGUCAGAAAAACCGCUUUUUGCGUUAUACGCGU
UAUGUAAACGCCUCCAAUCGGGAAGUUGUCAAGGAUUUGGAAAAACAGCAGA
AGGCUUUGGCGGUACAAGAGCAGAAAAUCAACAAUGAGCUUGCCCGUUUGAA
GAAAAUUCAGGCAAACGUGCAAUCCCUGCUGAAAAAACAGGGUGUAACCGAU
GCGGCGGAACAGACGGAAAGCCGCAGACAGAAUGCCAAAAUCUCCAAAGAUG
CCCGAAAACUGCUGGAACAGAAAGGGAACGAGCAGCAGCUGAACAAGCUCUU
GAGCAAUUUGGAGAAAAAAAAAGCCGAACACCGCAUUCAGGAUGCGGAAGCA
AAAAGAAAAUUGGCUGAAGCCAAACUGGCGGCAGCCGAAAAAGCCAGAAAAG
AAGCGGCGCAGCAGAAGGCUGAAGCGCGACGUGCGGAAAUGUCCAACCUGACC
GCCGAAGACAGGAACAUCCAAGCGCCUUCGGUUAUGGGUAUCGGCAGUGCCG
ACGGUUUCAGCCGCAUGCAGGGACGUUUGAAAAAACCGGUUGACGGUGUGCC
GACCGGGCUUUUCGGGCAGAACCGGAGCGGCGGCGAUGUUUGGAAAGGCGUG
UUCUAUUCCACUGCGCCUGCAACGGUUGAAAGCAUUGCGCCGGGAACGGUAA
GCUAUGCGGACGAGUUGGACGGCUACGGCAAAGUGGUCGUGAUCGAUCACGG
CGAGAACUACAUCAGCAUCUAUGCCGGUUUGAGCGAAAUUUCCGCCGGCAAG
GGUUAUACGGUCGCGGCAGGAAGCAAAAUCGGCACGAGCGGGUCGCUGCCGG
ACGGGGAAGAGGGGCUUUACCUGCAAAUACGUUAUCGAGGUCAGGUGUUGAA
CCCUUCGGGCUGGAUACGUUGA

cDNA: SEQ ID NO: 251

TCAACGTATCCAGCCCGAAGGGTTCAACACCTGACCTCGATAACGTATTTGCAG
GTAAAGCCCCTCTTCCCCGTCCGGCAGCGACCCGCTCGTGCCGATTTTGCTTCCT
GCCGCGACCGTATAACCCTTGCCGGCGGAAATTTCGCTCAAACCGGCATAGATG
CTGATGTAGTTCTCGCCGTGATCGATCACGACCACTTTGCCGTAGCCGTCCAACT
CGTCCGCATAGCTTACCGTTCCCGGCGCAATGCTTTCAACCGTTGCAGGCGCAGT
GGAATAGAACACGCCTTTCCAAACATCGCCGCCGCTCCGGTTCTGCCCGAAAAG
CCCGGTCGGCACACCGTCAACCGGTTTTTTCAAACGTCCCTGCATGCGGCTGAAA
CCGTCGGCACTGCCGATACCCATAACCGAAGGCGCTTGGATGTTCCTGTCTTCGG
CGGTCAGGTTGGACATTTCCGCACGTCGCGCTTCAGCCTTCTGCTGCGCCGCTTC
TTTTCTGGCTTTTTCGGCTGCCGCCAGTTTGGCTTCAGCCAATTTTCTTTTTGCTTC
CGCATCCTGAATGCGGTGTTCGGCTTTTTTTTTCTCCAAATTGCTCAAGAGCTTGT
TCAGCTGCTGCTCGTTCCCTTTCTGTTCCAGCAGTTTTCGGGCATCTTTGGAGATT
TTGGCATTCTGTCTGCGGCTTTCCGTCTGTTCCGCCGCATCGGTTACACCCTGTTT
TTTCAGCAGGGATTGCACGTTTGCCTGAATTTTCTTCAAACGGGCAAGCTCATTG
TTGATTTTCTGCTCTTGTACCGCCAAAGCCTTCTGCTGTTTTTCCAAATCCTTGAC
AACTTCCCGATTGGAGGCGTTTACATAACGCGTATAACGCAAAAAGCGGTTTTTC
TGACCCGGTTCGGCGTTTTTCAGGAACAGGGCAACCGCATTCGGCCGGCTGTTTT
TATAGTTCCCCGATACGAAACGGGAAATCTGCGCTTTCGTAGCGGCGACTTCCGT
TTTCAAACGGTTCAGCTCGGTATTGAGTTTTTGGAATTTGTCCCAAGCCTCGCGCT
GTTTGCGGTTGACGGAAGCAAGGTTGCCGCGCGCCTGACGGATACGCTCTTGGC
GGATACGTTCTTGGCGGATACGCTCTTCCTGAAGCTGTTTGAGGGAATTGCTGAC
ATGAAGCAGCATTCCTTCGCTTTGTTTGAGCAGGGCTTTTTTGTTTTCGACATCAT
TGGTGGCAGCGGCAACGGCGGCTTTCAATTCGTCGGAATCGGTTTTGGCATTTTT
CTCTTCCGTGTGTTTTTTGTCCTGTTTCACTGCTTTGCCGTTCTTGTCGGAACGGG
CTTTTTTATTTGCAGAAACAGTGTCTTTTTCCGCCTTGCCGCCCTTGCGCGGATTG
CCCTGTCCTTTTGCCCCTTTTTTGCCTTGTTTGTTTTCAGGCTGTGTTTTTGCGTTT
TTTTCTTTGGATCCGGACACGGGCTTGCCATGTGCCTTTTTGTGTTCCGCCTTCGA
TTCCTTATCCCCTTCGCGTCCTTTGCGCGCAGACTGCCTGGATGTCGCCTCCTTCT
CTGCTTCTTTGCGGTTTTTGGCGGTTTTTTTGAACTCTTTTGCCGCCTCTTTGCCGC
CTGTTTTTTTATCTTTCACTGCGGCATTTTTGCCTTTTTCTTTTTTGCCTTCCGCCG
CTTCGGGCTGTTCTTTTTTGTTCTTCGCCTGTTTTTTCACTTCGGCGGAACGGTTGT
GTGCCGCGTCGTGGGCGGCAACGGCGGGCGTGGAAAAAACGAGCATCAGGGCA
AGCAGAAGGGGTTTGTAGCGCAT

### 8. NGO0632: NC_002946.2:c622370-622050

DNA (- strand): SEQ ID NO: 252

ATGATTACCCTTACCGAGAATGCCGCAAAACACATCAATGACTATCTCGCCAAA
CGCGGCAAAGGCTTGGGCGTACGCTTGGGTGTAAAAACCAGCGGCTGCTCGGGG
ATGGCGTACAACCTTGAATTTGTCGATGAAGCCAACGGCGACGACCTGATTTTCG
AAGGACACGGCGCGCGCATTTATATCGACCCGAAAAGCTTGGTTTATCTGGACG
GCACACAAGTCGATTACACCAAAGAAGATTTGCAGGAAGGTTTCAAATTTGAAA
ACCCCAATGTCAAAGACTCCTGCGGCTGCGGCGAGAGCTTCCACGTTTAA

RNA: SEQ ID NO: 253

AUGAUUACCCUUACCGAGAAUGCCGCAAAACACAUCAAUGACUAUCUCGCCAA
ACGCGGCAAAGGCUUGGGCGUACGCUUGGGUGUAAAAACCAGCGGCUGCUCG
GGGAUGGCGUACAACCUUGAAUUUGUCGAUGAAGCCAACGGCGACGACCUGA
UUUUCGAAGGACACGGCGCGCGCAUUUAUAUCGACCCGAAAAGCUUGGUUUA
UCUGGACGGCACACAAGUCGAUUACACCAAAGAAGAUUUGCAGGAAGGUUUC
AAAUUUGAAAACCCCAAUGUCAAAGACUCCUGCGGCUGCGGCGAGAGCUUCCA
CGUUUAA

cDNA: SEQ ID NO: 254

TTAAACGTGGAAGCTCTCGCCGCAGCCGCAGGAGTCTTTGACATTGGGGTTTTCA
AATTTGAAACCTTCCTGCAAATCTTCTTTGGTGTAATCGACTTGTGTGCCGTCCAG
ATAAACCAAGCTTTTCGGGTCGATATAAATGCGCGCGCCGTGTCCTTCGAAAATC
AGGTCGTCGCCGTTGGCTTCATCGACAAATTCAAGGTTGTACGCCATCCCCGAGC
AGCCGCTGGTTTTTACACCCAAGCGTACGCCCAAGCCTTTGCCGCGTTTGGCGAG
ATAGTCATTGATGTGTTTTGCGGCATTCTCGGTAAGGGTAATCAT

### 9. NGO0633: NC_002946.2:c622843-622457

DNA (- strand): SEQ ID NO: 255

ATGGCATACAGCGATAAAGTAATCGACCACTACGAAAATCCCCGCAACGTCGGC
ACTTTCGACAAAAACGACGAGTCCGTCGGCACCGGCATGGTCGGCGCGCCCGCC
TGCGGCGACGTGATGCGCCTGCAAATCAAAGTGAACGATGAAGGCATCATCGAA
GATGCGAAATTCAAAACTTACGGCTGCGGTTCCGCCATCGCTTCGTCCAGCCTGA
TTACCGAGTGGGTCAAAGGCAAAAGTCTGGATGACGCGCTGGCAATCAAAAACA
GCGAAATCGCCGAAGAACTGGAATTGCCGCCGGTAAAAATCCACTGCTCCATCT
TGGCTGAAGATGCGGTAAAAGCGGCCGTTGCCGACTACCGCAAACGTCAGGAAA
ACAGATAA

RNA SEQ ID NO: 256

AUGGCAUACAGCGAUAAAGUAAUCGACCACUACGAAAAUCCCCGCAACGUCGG
CACUUUCGACAAAAACGACGAGUCCGUCGGCACCGGCAUGGUCGGCGCGCCCG
CCUGCGGCGACGUGAUGCGCCUGCAAAUCAAAGUGAACGAUGAAGGCAUCAU
CGAAGAUGCGAAAUUCAAAACUUACGGCUGCGGUUCCGCCAUCGCUUCGUCCA
GCCUGAUUACCGAGUGGGUCAAAGGCAAAAGUCUGGAUGACGCGCUGGCAAU
CAAAAACAGCGAAAUCGCCGAAGAACUGGAAUUGCCGCCGGUAAAAAUCCAC
UGCUCCAUCUUGGCUGAAGAUGCGGUAAAAGCGGCCGUUGCCGACUACCGCAA
ACGUCAGGAAAACAGAUAA

cDNA: SEQ ID NO: 257

TTATCTGTTTTCCTGACGTTTGCGGTAGTCGGCAACGGCCGCTTTTACCGCATCTT
CAGCCAAGATGGAGCAGTGGATTTTTACCGGCGGCAATTCCAGTTCTTCGGCGAT
TTCGCTGTTTTTGATTGCCAGCGCGTCATCCAGACTTTTGCCTTTGACCCACTCGG
TAATCAGGCTGGACGAAGCGATGGCGGAACCGCAGCCGTAAGTTTTGAATTTCG
CATCTTCGATGATGCCTTCATCGTTCACTTTGATTTGCAGGCGCATCACGTCGCCG
CAGGCGGGCGCGCCGACCATGCCGGTGCCGACGGACTCGTCGTTTTTGTCGAAA
GTGCCGACGTTGCGGGGATTTTCGTAGTGGTCGATTACTTTATCGCTGTATGCCA
T

### 10. NGO0678: NC_002946.2:c667233-666979

DNA (- strand): SEQ ID NO: 258

ATGAACAAACTTTTCGTTACCGCCCTGTCCGCCCTCGCCTTGTCCGCCTGCGCCG
GCACTTGGCAGGGCGCGAAACAAGACACCGCCCGCAACCTTGACAAAACACAG
GCCGCCGCCGAACGCGCCGCCGAACAAACAGGCAACGCCGTCGAAAAAGGTTG
GGACAAAACCAAAGAAGCCGTCAAAAAAGGCGGCAATGCCGTCGGACGCGGCA
TTTCCCATCTCGGCAAAAAAATCGAAAACGCCACCGAATAA

RNA SEQ ID NO: 259

AUGAACAAACUUUUCGUUACCGCCCUGUCCGCCCUCGCCUUGUCCGCCUGCGC
CGGCACUUGGCAGGGCGCGAAACAAGACACCGCCCGCAACCUUGACAAAACAC
AGGCCGCCGCCGAACGCGCCGCCGAACAAACAGGCAACGCCGUCGAAAAAGGU
UGGGACAAAACCAAAGAAGCCGUCAAAAAAGGCGGCAAUGCCGUCGGACGCG
GCAUUUCCCAUCUCGGCAAAAAAAUCGAAAACGCCACCGAAUAA

cDNA: SEQ ID NO: 260

TTATTCGGTGGCGTTTTCGATTTTTTTGCCGAGATGGGAAATGCCGCGTCCGACG
GCATTGCCGCCTTTTTTGACGGCTTCTTTGGTTTTGTCCCAACCTTTTTCGACGGC
GTTGCCTGTTTGTTCGGCGGCGCGTTCGGCGGCGGCCTGTGTTTTGTCAAGGTTG
CGGGCGGTGTCTTGTTTCGCGCCCTGCCAAGTGCCGGCGCAGGCGGACAAGGCG
AGGGCGGACAGGGCGGTAACGAAAAGTTTGTTCAT

### 11. NGO0926: NC_002946.2:c906814-906077

DNA (- strand): SEQ ID NO: 261

ATGGCTTTGCAAGATCGTACCGGTCAAAAAGTACCTTCCGTAGTATTCCGCACCC
GCGTCGGCGACACTTGGAAAGATGTGTCTACCGATGATTTGTTCAAAGGCAAAA
AAGTAGTCGTATTCTCCCTGCCCGGTGCATTTACCCCGACTTGTTCTTCTTCACAC
CTGCCGCGTTACAACGAATTGTTCGGCGCGTTCAAAGAAAACGGCGTTGACGCA
ATCTGCTGCGTATCTGTAAACGATACTTTCGTAATGAACGCTTGGGCTGCCGAAG
AAGAATCAGACAACATCTACATGATTCCTGACGGCAACGGCGAATTTACCGAAG
GTATGGGTATGCTGGTCGGTAAAGAAGACTTGGGCTTCGGCAAACGCTCTTGGC
GTTACTCCATGCTGGTTAACGACGGCGTGGTTGAAAAAATGTTCATCGAACCTGA
AGAACCGGGCGATCCTTTCAAAGTATCCGATGCAGATACTATGCTGAAATTCGTT
GCTCCCGATTGGAAGGCTCAAGAGTCTGTGGCAATTTTCACTAAACCAGGTTGCC
AATTCTGTGCCAAAGTCAAACAAGCTTTGCAAGACAAAGGTTTGTCTTACGAAG
AAATCGTATTGGGCAAAGATGCAACCGTTACTTCCGTTCGCGCTATTACCGGCAA
GATGACTGCCCCTCAAGTCTTCATCGGCGGCAAATACATCGGCGGCAGCGAAGA
TTTGGAAGCTTACTTGGCTAAAAACTGA

RNA: SEQ ID NO: 262

AUGGCUUUGCAAGAUCGUACCGGUCAAAAAGUACCUUCCGUAGUAUUCCGCA
CCCGCGUCGGCGACACUUGGAAAGAUGUGUCUACCGAUGAUUUGUUCAAAGG
CAAAAAAGUAGUCGUAUUCUCCCUGCCCGGUGCAUUUACCCCGACUUGUUCUU
CUUCACACCUGCCGCGUUACAACGAAUUGUUCGGCGCGUUCAAAGAAAACGGC
GUUGACGCAAUCUGCUGCGUAUCUGUAAACGAUACUUUCGUAAUGAACGCUU
GGGCUGCCGAAGAAGAAUCAGACAACAUCUACAUGAUUCCUGACGGCAACGG
CGAAUUUACCGAAGGUAUGGGUAUGCUGGUCGGUAAAGAAGACUUGGGCUUC
GGCAAACGCUCUUGGCGUUACUCCAUGCUGGUUAACGACGGCGUGGUUGAAA
AAAUGUUCAUCGAACCUGAAGAACCGGGCGAUCCUUUCAAAGUAUCCGAUGC
AGAUACUAUGCUGAAAUUCGUUGCUCCCGAUUGGAAGGCUCAAGAGUCUGUG
GCAAUUUUCACUAAACCAGGUUGCCAAUUCUGUGCCAAAGUCAAACAAGCUU
UGCAAGACAAAGGUUUGUCUUACGAAGAAAUCGUAUUGGGCAAAGAUGCAAC
CGUUACUUCCGUUCGCGCUAUUACCGGCAAGAUGACUGCCCCUCAAGUCUUCA
UCGGCGGCAAAUACAUCGGCGGCAGCGAAGAUUUGGAAGCUUACUUGGCUAA
AAACUGA

cDNA: SEQ ID NO: 263

TCAGTTTTTAGCCAAGTAAGCTTCCAAATCTTCGCTGCCGCCGATGTATTTGCCG
CCGATGAAGACTTGAGGGGCAGTCATCTTGCCGGTAATAGCGCGAACGGAAGTA
ACGGTTGCATCTTTGCCCAATACGATTTCTTCGTAAGACAAACCTTTGTCTTGCA
AAGCTTGTTTGACTTTGGCACAGAATTGGCAACCTGGTTTAGTGAAAATTGCCAC
AGACTCTTGAGCCTTCCAATCGGGAGCAACGAATTTCAGCATAGTATCTGCATCG
GATACTTTGAAAGGATCGCCCGGTTCTTCAGGTTCGATGAACATTTTTTCAACCA
CGCCGTCGTTAACCAGCATGGAGTAACGCCAAGAGCGTTTGCCGAAGCCCAAGT
CTTCTTTACCGACCAGCATACCCATACCTTCGGTAAATTCGCCGTTGCCGTCAGG
AATCATGTAGATGTTGTCTGATTCTTCTTCGGCAGCCCAAGCGTTCATTACGAAA
GTATCGTTTACAGATACGCAGCAGATTGCGTCAACGCCGTTTTCTTTGAACGCGC
CGAACAATTCGTTGTAACGCGGCAGGTGTGAAGAAGAACAAGTCGGGGTAAATG
CACCGGGCAGGGAGAATACGACTACTTTTTTGCCTTTGAACAAATCATCGGTAGA
CACATCTTTCCAAGTGTCGCCGACGCGGGTGCGGAATACTACGGAAGGTACTTTT
TGACCGGTACGATCTTGCAAAGCCAT

### 12. NGO0936: NC_002946.2:c914813-914253

DNA (- strand): SEQ ID NO: 264

ATGAAAACAGCACAAGAACTGCGCGCCGGCAATGTATTTATGGTCGGCAACGAT
CCTATGGTCGTTCAAAAAACCGAATACATCAAAGGCGGCCGCTCTTCCGCCAAA
GTCAGCATGAAACTGAAAAACCTGCTGACCGGCGCTGCTTCCGAAACCATTTAC
AAAGCCGACGACAAATTCGACGTGGTCATCCTGTCCCGCAAAAACTGTACGTAC
AGCTATTTTGCCGACCCGATGTACGTCTTTATGGACGAAGAATTCAACCAATACG
AAATCGAAGCCGACAACATCGGCGACGCGTTGAAATTCATCGTTGACGGTATGG
AAGACCAATGCGAAGTTACCTTCTATGAAGGCAATCCCATTTCTGTCGAACTGCC
CACCATCATCGTGCGCGAAGTCGAGTACACCGAGCCTGCCGTCAAAGGCGATAC
TTCCGGCAAAGTGATGAAAACCGCGCGTCTGGTCGGCGGCACCGAAATCCAAGT
GATGTCTTACATCGAAAACGGCGACAAAGTCGAAATCGATACCCGTACCGGCGA
ATTCCGCAAACGCGCCTGA

RNA: SEQ ID NO: 265

AUGAAAACAGCACAAGAACUGCGCGCCGGCAAUGUAUUUAUGGUCGGCAACG
AUCCUAUGGUCGUUCAAAAAACCGAAUACAUCAAAGGCGGCCGCUCUUCCGCC
AAAGUCAGCAUGAAACUGAAAAACCUGCUGACCGGCGCUGCUUCCGAAACCAU
UUACAAAGCCGACGACAAAUUCGACGUGGUCAUCCUGUCCCGCAAAAACUGUA
CGUACAGCUAUUUUGCCGACCCGAUGUACGUCUUUAUGGACGAAGAAUUCAA
CCAAUACGAAAUCGAAGCCGACAACAUCGGCGACGCGUUGAAAUUCAUCGUU
GACGGUAUGGAAGACCAAUGCGAAGUUACCUUCUAUGAAGGCAAUCCCAUUU
CUGUCGAACUGCCCACCAUCAUCGUGCGCGAAGUCGAGUACACCGAGCCUGCC
GUCAAAGGCGAUACUUCCGGCAAAGUGAUGAAAACCGCGCGUCUGGUCGGCG
GCACCGAAAUCCAAGUGAUGUCUUACAUCGAAAACGGCGACAAAGUCGAAAU
CGAUACCCGUACCGGCGAAUUCCGCAAACGCGCCUGA

cDNA: SEQ ID NO: 266

TCAGGCGCGTTTGCGGAATTCGCCGGTACGGGTATCGATTTCGACTTTGTCGCCG
TTTTCGATGTAAGACATCACTTGGATTTCGGTGCCGCCGACCAGACGCGCGGTTT
TCATCACTTTGCCGGAAGTATCGCCTTTGACGGCAGGCTCGGTGTACTCGACTTC
GCGCACGATGATGGTGGGCAGTTCGACAGAAATGGGATTGCCTTCATAGAAGGT
AACTTCGCATTGGTCTTCCATACCGTCAACGATGAATTTCAACGCGTCGCCGATG
TTGTCGGCTTCGATTTCGTATTGGTTGAATTCTTCGTCCATAAAGACGTACATCGG
GTCGGCAAAATAGCTGTACGTACAGTTTTTGCGGGACAGGATGACCACGTCGAA
TTTGTCGTCGGCTTTGTAAATGGTTTCGGAAGCAGCGCCGGTCAGCAGGTTTTTC
AGTTTCATGCTGACTTTGGCGGAAGAGCGGCCGCCTTTGATGTATTCGGTTTTTT
GAACGACCATAGGATCGTTGCCGACCATAAATACATTGCCGGCGCGCAGTTCTT
GTGCTGTTTTCAT

### 13. NGO0950a: NC_002946.2:925084-925782

DNA (+ strand): SEQ ID NO: 267

GTGCAGGCGGATTTAGCCTACGCCGCCGAACGCATTACCCACGATTATCCGGAA
CCAACCGGTGCAAAAAAAGACAAAAAAATAAGCACGGTAAGCGATTATTTCAG
AAACATCCGTACGCATTCCGTCCACCCCAGGGTGTCGGTCGGCTACGATTTCGGC
AGCTGGAGGATAGCGGCAGATTATGCCCGTTACAGAAAGTGGAACAACAGTAAA
TATTCCGTCAACATAAAAAGGGTGAAAGAAAACAATGGCAGCGGGAAAAAACT
GACGCAAGACCTGAAGACGGAAAATCAGGAAAACGGTACGTTCCACGCCGTTTC
TTCTCTCGGCTTGTCCGCCGTTTACGATTTCGATACCGGTTCCCGCTTCAAACCCT
ATGCAGGCGTGCGCGTCAGCTACGGACACGTCAGACACAGCATCGATTCGACCA
AAAAAACAACAGATGTTATTACCGCCCCCCCCACTACTTCTGACGGAGCACCTA
CAACTTATAATGCTAATCCACAGACGCAAAACCCTTATCACCAAAGCGACAGCA
TCCGCCGCGTGGGCCTCGGCGTCATCGCCGGCGTCGGTTTCGACATCACGCCCAA
CCTGACCCTGGACACCGGCTACCGCTACCACAACTGGGGACGCCTGGAAAACAC
CCGCTTCAAAACCCACGAAGCCTCATTGGGCATGCGCTACCGCTTCTGA

RNA: SEQ ID NO: 268

GUGCAGGCGGAUUUAGCCUACGCCGCCGAACGCAUUACCCACGAUUAUCCGGA
ACCAACCGGUGCAAAAAAAGACAAAAAAAUAAGCACGGUAAGCGAUUAUUUC
AGAAACAUCCGUACGCAUUCCGUCCACCCCAGGGUGUCGGUCGGCUACGAUUU
CGGCAGCUGGAGGAUAGCGGCAGAUUAUGCCCGUUACAGAAAGUGGAACAAC
AGUAAAUAUUCCGUCAACAUAAAAAGGGUGAAAGAAAACAAUGGCAGCGGGA
AAAAACUGACGCAAGACCUGAAGACGGAAAAUCAGGAAAACGGUACGUUCCA
CGCCGUUUCUUCUCUCGGCUUGUCCGCCGUUUACGAUUUCGAUACCGGUUCCC
GCUUCAAACCCUAUGCAGGCGUGCGCGUCAGCUACGGACACGUCAGACACAGC
AUCGAUUCGACCAAAAAAACAACAGAUGUUAUUACCGCCCCCCCCACUACUUC
UGACGGAGCACCUACAACUUAUAAUGCUAAUCCACAGACGCAAAACCCUUAUC
ACCAAAGCGACAGCAUCCGCCGCGUGGGCCUCGGCGUCAUCGCCGGCGUCGGU
UUCGACAUCACGCCCAACCUGACCCUGGACACCGGCUACCGCUACCACAACUG
GGGACGCCUGGAAAACACCCGCUUCAAAACCCACGAAGCCUCAUUGGGCAUGC
GCUACCGCUUCUGA

cDNA: SEQ ID NO: 269

TCAGAAGCGGTAGCGCATGCCCAATGAGGCTTCGTGGGTTTTGAAGCGGGTGTTT
TCCAGGCGTCCCCAGTTGTGGTAGCGGTAGCCGGTGTCCAGGGTCAGGTTGGGC
GTGATGTCGAAACCGACGCCGGCGATGACGCCGAGGCCCACGCGGCGGATGCTG
TCGCTTTGGTGATAAGGGTTTTGCGTCTGTGGATTAGCATTATAAGTTGTAGGTG
CTCCGTCAGAAGTAGTGGGGGGGGCGGTAATAACATCTGTTGTTTTTTTGGTCGA
ATCGATGCTGTGTCTGACGTGTCCGTAGCTGACGCGCACGCCTGCATAGGGTTTG
AAGCGGGAACCGGTATCGAAATCGTAAACGGCGGACAAGCCGAGAGAAGAAAC
GGCGTGGAACGTACCGTTTTCCTGATTTTCCGTCTTCAGGTCTTGCGTCAGTTTTT
TCCCGCTGCCATTGTTTTCTTTCACCCTTTTTATGTTGACGGAATATTTACTGTTGT
TCCACTTTCTGTAACGGGCATAATCTGCCGCTATCCTCCAGCTGCCGAAATCGTA
GCCGACCGACACCCTGGGGTGGACGGAATGCGTACGGATGTTTCTGAAATAATC
GCTTACCGTGCTTATTTTTTTGTCTTTTTTTGCACCGGTTGGTTCCGGATAATCGT
GGGTAATGCGTTCGGCGGCGTAGGCTAAATCCGCCTGCAC

### 14. NGO1040a: NC_002946.2:c1000440-999760

DNA (- strand): SEQ ID NO: 270

GTGCAGGCGGATCTGGCTTACGCCTACGAGCACATCACCCGCGATTATCCCGAT
GCAGCCGGTGCAAACCAAGGCAAAAAAATAAGCACGGTAAGCGATTATTTCAAA
AACATCCGTACGCATTCCATCCACCCCAGGGTGTCGGTCGGCTACGACTTCGGCG
GCTGGAGGATAGCGGCAGATTATGCCCGTTACAGAAAGTGGAACGACAATAAAT
ATTCCGTCGACATAAAAGAGTTGGAAAACAAGAATCAGAATAAGAGAGACCTG
AAGACGGAAAATCAGGAAAACGGCAGCTTCCACGCCGTTTCTTCTCTCGGCTTAT
CAGCCGTTTACGATTTCAAACTCAACGACAAATTCAAACCCTATATCGGTGCGCG
CGTCGCCTACGGACACGTCAGACACAGCATCGATTCGACCAAAAAAACAACAGA
GTTTCTTACCGCCGCCGGTCAGGATGGCGGAGCGCCTACGGTTTATAATAACGGA
AGTACGCAAGACGCCCATCAAGAAAGCGACAGCATCCGCCGCGTGGGCCTCGGC
GTCATCGCCGGTATCGGTTTCGACATCACGCCCAAGCTGACCCTGGACACCGGCT
ACCGCTACCACAACTGGGGACGCTTGGAAAACACCCGCTTCAAAACCCACGAAG
CCTCATTGGGCGTGCGCTACCGCTTCTGA

RNA: SEQ ID NO: 271

GUGCAGGCGGAUCUGGCUUACGCCUACGAGCACAUCACCCGCGAUUAUCCCGA
UGCAGCCGGUGCAAACCAAGGCAAAAAAAUAAGCACGGUAAGCGAUUAUUUC
AAAAACAUCCGUACGCAUUCCAUCCACCCCAGGGUGUCGGUCGGCUACGACUU
CGGCGGCUGGAGGAUAGCGGCAGAUUAUGCCCGUUACAGAAAGUGGAACGAC
AAUAAAUAUUCCGUCGACAUAAAAGAGUUGGAAAACAAGAAUCAGAAUAAGA
GAGACCUGAAGACGGAAAAUCAGGAAAACGGCAGCUUCCACGCCGUUUCUUC
UCUCGGCUUAUCAGCCGUUUACGAUUUCAAACUCAACGACAAAUUCAAACCCU
AUAUCGGUGCGCGCGUCGCCUACGGACACGUCAGACACAGCAUCGAUUCGACC
AAAAAAACAACAGAGUUUCUUACCGCCGCCGGUCAGGAUGGCGGAGCGCCUAC
GGUUUAUAAUAACGGAAGUACGCAAGACGCCCAUCAAGAAAGCGACAGCAUC
CGCCGCGUGGGCCUCGGCGUCAUCGCCGGUAUCGGUUUCGACAUCACGCCCAA
GCUGACCCUGGACACCGGCUACCGCUACCACAACUGGGGACGCUUGGAAAACA
CCCGCUUCAAAACCCACGAAGCCUCAUUGGGCGUGCGCUACCGCUUCUGA

cDNA: SEQ ID NO: 272

TCAGAAGCGGTAGCGCACGCCCAATGAGGCTTCGTGGGTTTTGAAGCGGGTGTT
TTCCAAGCGTCCCCAGTTGTGGTAGCGGTAGCCGGTGTCCAGGGTCAGCTTGGGC
GTGATGTCGAAACCGATACCGGCGATGACGCCGAGGCCCACGCGGCGGATGCTG
TCGCTTTCTTGATGGGCGTCTTGCGTACTTCCGTTATTATAAACCGTAGGCGCTCC
GCCATCCTGACCGGCGGCGGTAAGAAACTCTGTTGTTTTTTTGGTCGAATCGATG
CTGTGTCTGACGTGTCCGTAGGCGACGCGCGCACCGATATAGGGTTTGAATTTGT
CGTTGAGTTTGAAATCGTAAACGGCTGATAAGCCGAGAGAAGAAACGGCGTGGA
AGCTGCCGTTTTCCTGATTTTCCGTCTTCAGGTCTCTCTTATTCTGATTCTTGTTTT
CCAACTCTTTTATGTCGACGGAATATTTATTGTCGTTCCACTTTCTGTAACGGGCA
TAATCTGCCGCTATCCTCCAGCCGCCGAAGTCGTAGCCGACCGACACCCTGGGGT
GGATGGAATGCGTACGGATGTTTTTGAAATAATCGCTTACCGTGCTTATTTTTTTG
CCTTGGTTTGCACCGGCTGCATCGGGATAATCGCGGGTGATGTGCTCGTAGGCGT
AAGCCAGATCCGCCTGCAC

### 15. NGO1073a: NC_002946.2:1035309-1035998

DNA (+ strand): SEQ ID NO: 273

GTGCAGGCGGATTTAGCCTACGCCGCCGAACGCATTACCCACGATTATCCGGAA
CCAACCGGTACAAAAAAAGACAAAATAAGCACGGTAAGCGATTATTTCAGAAAC
ATCCGTACGCATTCCATCCACCCCAGGGTGTCGGTCGGCTACGACTTCGGCGGCT
GGAGGATAGCGGCAGATTATGCCCGTTACAGAAAGTGGAACAACAGTAAATATT
CCGTCAACACAAAAAAGGTGAACGAAAACAAGGGCGAAAAGATAAACGTGACG
CAATATCTGAAGGCGGAAAATCAGGAAAACGGTACGTTCCACGCCGTTTCTTCTC
TCGGCTTGTCCGCCGTTTACGATTTCAAACTCAACGACAAATTCAAACCCTATAT
CGGTGCGCGCGTCGCCTACGGACACGTCAGACACAGCATCGATTCGACCAAAAA
AACAACAGAGTTTCTTACCGCCGCCGGTCAGGATGGCGGAGCGCCTACGGTTTA
TAATAACGGAAGTACGCAAGACGCCCATCAAGAAAGCGACAGCATCCGCCGCGT
GGGCCTCGGCGTCATCGCCGGCGTCGGTTTCGACATCACGCCCAACCTGACCTTG
GACGCCGGGTACCGCTACCACAACTGGGGACGCTTGGAAAACACCCGCTTCAAA
ACCCACGAAGCCTCGTTGGGCATGCGCTACCGCTTCTGA

RNA: SEQ ID NO: 274

GUGCAGGCGGAUUUAGCCUACGCCGCCGAACGCAUUACCCACGAUUAUCCGGA
ACCAACCGGUACAAAAAAAGACAAAAUAAGCACGGUAAGCGAUUAUUUCAGA
AACAUCCGUACGCAUUCCAUCCACCCCAGGGUGUCGGUCGGCUACGACUUCGG
CGGCUGGAGGAUAGCGGCAGAUUAUGCCCGUUACAGAAAGUGGAACAACAGU
AAAUAUUCCGUCAACACAAAAAAGGUGAACGAAAACAAGGGCGAAAAGAUAA
ACGUGACGCAAUAUCUGAAGGCGGAAAAUCAGGAAAACGGUACGUUCCACGC
CGUUUCUUCUCUCGGCUUGUCCGCCGUUUACGAUUUCAAACUCAACGACAAAU
UCAAACCCUAUAUCGGUGCGCGCGUCGCCUACGGACACGUCAGACACAGCAUC
GAUUCGACCAAAAAAACAACAGAGUUUCUUACCGCCGCCGGUCAGGAUGGCG
GAGCGCCUACGGUUUAUAAUAACGGAAGUACGCAAGACGCCCAUCAAGAAAG
CGACAGCAUCCGCCGCGUGGGCCUCGGCGUCAUCGCCGGCGUCGGUUUCGACA
UCACGCCCAACCUGACCUUGGACGCCGGGUACCGCUACCACAACUGGGGACGC
UUGGAAAACACCCGCUUCAAAACCCACGAAGCCUCGUUGGGCAUGCGCUACCG
CUUCUGA

cDNA: SEQ ID NO: 275

TCAGAAGCGGTAGCGCATGCCCAACGAGGCTTCGTGGGTTTTGAAGCGGGTGTT
TTCCAAGCGTCCCCAGTTGTGGTAGCGGTACCCGGCGTCCAAGGTCAGGTTGGG
CGTGATGTCGAAACCGACGCCGGCGATGACGCCGAGGCCCACGCGGCGGATGCT
GTCGCTTTCTTGATGGGCGTCTTGCGTACTTCCGTTATTATAAACCGTAGGCGCTC
CGCCATCCTGACCGGCGGCGGTAAGAAACTCTGTTGTTTTTTTGGTCGAATCGAT
GCTGTGTCTGACGTGTCCGTAGGCGACGCGCGCACCGATATAGGGTTTGAATTTG
TCGTTGAGTTTGAAATCGTAAACGGCGGACAAGCCGAGAGAAGAAACGGCGTGG
AACGTACCGTTTTCCTGATTTTCCGCCTTCAGATATTGCGTCACGTTTATCTTTTC
GCCCTTGTTTTCGTTCACCTTTTTTGTGTTGACGGAATATTTACTGTTGTTCCACTT
TCTGTAACGGGCATAATCTGCCGCTATCCTCCAGCCGCCGAAGTCGTAGCCGACC
GACACCCTGGGGTGGATGGAATGCGTACGGATGTTTCTGAAATAATCGCTTACC
GTGCTTATTTTGTCTTTTTTTGTACCGGTTGGTTCCGGATAATCGTGGGTAATGCG
TTCGGCGGCGTAGGCTAAATCCGCCTGCAC

### 16. NGO1225: NC_002946.2:c1175547-1174729

DNA (- strand): SEQ ID NO: 276

ATGAACACCATTTTCAAAATCAGCGCACTGACCCTTTCCGCCGCTTTGGCACTTT
CCGCCTGCGGCAAAAAAGAAGCCGCCCCCGCATCTGCATCCGAACCTGCCGCCG
CTTCTGCCGCGCAGGGCGACACCTCTTCAATCGGCAGCACGATGCAGCAGGCAA
GCTATGCAATGGGCGTGGACATCGGACGCTCCCTGAAACAAATGAAGGAACAGG
GCGCGGAAATCGATTTGAAAGTCTTTACCGATGCCATGCAGGCAGTGTATGACG
GCAAAGAAATCAAAATGACCGAAGAGCAGGCCCAGGAAGTGATGATGAAATTC
CTGCAGGAGCAGCAGGCTAAAGCCGTAGAAAAACACAAGGCGGATGCGAAGGC
CAACAAAGAAAAAGGCGAAGCCTTCCTGAAGGAAAATGCCGCCAAAGACGGCG
TGAAGACCACTGCTTCCGGTCTGCAGTACAAAATCACCAAACAGGGTGAAGGCA
AACAGCCGACAAAAGACGACATCGTTACCGTGGAATACGAAGGCCGCCTGATTG
ACGGTACCGTATTCGACAGCAGCAAAGCCAACGGCGGCCCGGCCACCTTCCCTT
TGAGCCAAGTGATTCCGGGTTGGACCGAAGGCGTACGGCTTCTGAAAGAAGGCG
GCGAAGCCACGTTCTACATCCCGTCCAACCTTGCCTACCGCGAACAGGGTGCGG
GCGAAAAAATCGGTCCGAACGCCACTTTGGTATTTGACGTGAAACTGGTCAAAA
TCGGCGCACCCGAAAACGCGCCCGCCAAGCAGCCGGATCAAGTCGACATCAAAA
AAGTAAATTAA

RNA: SEQ ID NO: 277

AUGAACACCAUUUUCAAAAUCAGCGCACUGACCCUUUCCGCCGCUUUGGCACU
UUCCGCCUGCGGCAAAAAAGAAGCCGCCCCCGCAUCUGCAUCCGAACCUGCCG
CCGCUUCUGCCGCGCAGGGCGACACCUCUUCAAUCGGCAGCACGAUGCAGCAG
GCAAGCUAUGCAAUGGGCGUGGACAUCGGACGCUCCCUGAAACAAAUGAAGG
AACAGGGCGCGGAAAUCGAUUUGAAAGUCUUUACCGAUGCCAUGCAGGCAGU
GUAUGACGGCAAAGAAAUCAAAAUGACCGAAGAGCAGGCCCAGGAAGUGAUG
AUGAAAUUCCUGCAGGAGCAGCAGGCUAAAGCCGUAGAAAAACACAAGGCGG
AUGCGAAGGCCAACAAAGAAAAAGGCGAAGCCUUCCUGAAGGAAAAUGCCGC
CAAAGACGGCGUGAAGACCACUGCUUCCGGUCUGCAGUACAAAAUCACCAAAC
AGGGUGAAGGCAAACAGCCGACAAAAGACGACAUCGUUACCGUGGAAUACGA
AGGCCGCCUGAUUGACGGUACCGUAUUCGACAGCAGCAAAGCCAACGGCGGCC
CGGCCACCUUCCCUUUGAGCCAAGUGAUUCCGGGUUGGACCGAAGGCGUACGG
CUUCUGAAAGAAGGCGGCGAAGCCACGUUCUACAUCCCGUCCAACCUUGCCUA
CCGCGAACAGGGUGCGGGCGAAAAAAUCGGUCCGAACGCCACUUUGGUAUUU
GACGUGAAACUGGUCAAAAUCGGCGCACCCGAAAACGCGCCCGCCAAGCAGCC
GGAUCAAGUCGACAUCAAAAAAGUAAAUUAA

cDNA: SEQ ID NO: 278

TTAATTTACTTTTTTGATGTCGACTTGATCCGGCTGCTTGGCGGGCGCGTTTTCGG
GTGCGCCGATTTTGACCAGTTTCACGTCAAATACCAAAGTGGCGTTCGGACCGAT
TTTTTCGCCCGCACCCTGTTCGCGGTAGGCAAGGTTGGACGGGATGTAGAACGTG
GCTTCGCCGCCTTCTTTCAGAAGCCGTACGCCTTCGGTCCAACCCGGAATCACTT
GGCTCAAAGGGAAGGTGGCCGGGCCGCCGTTGGCTTTGCTGCTGTCGAATACGG
TACCGTCAATCAGGCGGCCTTCGTATTCCACGGTAACGATGTCGTCTTTTGTCGG
CTGTTTGCCTTCACCCTGTTTGGTGATTTTGTACTGCAGACCGGAAGCAGTGGTCT
TCACGCCGTCTTTGGCGGCATTTTCCTTCAGGAAGGCTTCGCCTTTTTCTTTGTTG
GCCTTCGCATCCGCCTTGTGTTTTTCTACGGCTTTAGCCTGCTGCTCCTGCAGGAA
TTTCATCATCACTTCCTGGGCCTGCTCTTCGGTCATTTTGATTTCTTTGCCGTCATA
CACTGCCTGCATGGCATCGGTAAAGACTTTCAAATCGATTTCCGCGCCCTGTTCC
TTCATTTGTTTCAGGGAGCGTCCGATGTCCACGCCCATTGCATAGCTTGCCTGCT
GCATCGTGCTGCCGATTGAAGAGGTGTCGCCCTGCGCGGCAGAAGCGGCGGCAG
GTTCGGATGCAGATGCGGGGGCGGCTTCTTTTTTGCCGCAGGCGGAAAGTGCCA
AAGCGGCGGAAAGGGTCAGTGCGCTGATTTTGAAAATGGTGTTCAT

### 17. NGO1277a: NC_002946.2:1231620-1232324

DNA (+ strand): SEQ ID NO: 279

GTGCAGGCGGATTTAGCCTACGCCGCCGAACGCATTACCCACGATTATCCGGAA
CCAACCGGTGCAAAAAAAGGCAAAATAAGCACGGTAAGCGATTATTTCAGAAAC
ATCCGTACGCATTCCATCCACCCCAGGGTGTCGGTCGGCTACGACTTCGGCGGCT
GGAGGATAGCGGCAGATTATGCCCGTTACAGAAAGTGGAACGACAATAAATATT
CCGTGAACATAAAAGAGTTGGGAAGAAAGGATGGTACCTCTTCTAGCGGCCGCT
ATCTTAACATACAAACCCGAAAGACGGAAAATCAGGAAAACGGTACGTTCCACG
CCGTTTCTTCTCTCGGCTTGTCAACCGTTTACGATTTCAGAGCCAACGATAAATTC
AAACCCTATATCGGCGTGCGCGTCGCCTACGGACACGTCAGACATCAGGTTCATT
CAATGGAAAAAGAAACCACGACTGTTACCACTTACCCAAGCGACGGTAGTGCGA
AAACTTCTGTTCCATCAGAAATGCCCCCCAAACCTGCCTATCACGAAAACCGCA
GCAGCCGCCGCTTGGGCTTCGGCGCGATGGCGGGCGTGGGCATAGACGTCGCGC
CCGGTCTGACCTTGGACGCCGGCTACCGCTACCACTATTGGGGACGCCTGGAAA
ACACCCGCTTCAAAACCCACGAAGCCTCATTGGGCATGCGCTACCGCTTCTGA

RNA: SEQ ID NO: 280

GUGCAGGCGGAUUUAGCCUACGCCGCCGAACGCAUUACCCACGAUUAUCCGGA
ACCAACCGGUGCAAAAAAAGGCAAAAUAAGCACGGUAAGCGAUUAUUUCAGA
AACAUCCGUACGCAUUCCAUCCACCCCAGGGUGUCGGUCGGCUACGACUUCGG
CGGCUGGAGGAUAGCGGCAGAUUAUGCCCGUUACAGAAAGUGGAACGACAAU
AAAUAUUCCGUGAACAUAAAAGAGUUGGGAAGAAAGGAUGGUACCUCUUCUA
GCGGCCGCUAUCUUAACAUACAAACCCGAAAGACGGAAAAUCAGGAAAACGG
UACGUUCCACGCCGUUUCUUCUCUCGGCUUGUCAACCGUUUACGAUUUCAGAG
CCAACGAUAAAUUCAAACCCUAUAUCGGCGUGCGCGUCGCCUACGGACACGUC
AGACAUCAGGUUCAUUCAAUGGAAAAAGAAACCACGACUGUUACCACUUACC
CAAGCGACGGUAGUGCGAAAACUUCUGUUCCAUCAGAAAUGCCCCCCAAACCU
GCCUAUCACGAAAACCGCAGCAGCCGCCGCUUGGGCUUCGGCGCGAUGGCGGG
CGUGGGCAUAGACGUCGCGCCCGGUCUGACCUUGGACGCCGGCUACCGCUACC
ACUAUUGGGGACGCCUGGAAAACACCCGCUUCAAAACCCACGAAGCCUCAUUG
GGCAUGCGCUACCGCUUCUGA

cDNA: SEQ ID NO: 281

TCAGAAGCGGTAGCGCATGCCCAATGAGGCTTCGTGGGTTTTGAAGCGGGTGTTT
TCCAGGCGTCCCCAATAGTGGTAGCGGTAGCCGGCGTCCAAGGTCAGACCGGGC
GCGACGTCTATGCCCACGCCCGCCATCGCGCCGAAGCCCAAGCGGCGGCTGCTG
CGGTTTTCGTGATAGGCAGGTTTGGGGGGCATTTCTGATGGAACAGAAGTTTTCG
CACTACCGTCGCTTGGGTAAGTGGTAACAGTCGTGGTTTCTTTTTCCATTGAATG
AACCTGATGTCTGACGTGTCCGTAGGCGACGCGCACGCCGATATAGGGTTTGAA
TTTATCGTTGGCTCTGAAATCGTAAACGGTTGACAAGCCGAGAGAAGAAACGGC
GTGGAACGTACCGTTTTCCTGATTTTCCGTCTTTCGGGTTTGTATGTTAAGATAGC
GGCCGCTAGAAGAGGTACCATCCTTTCTTCCCAACTCTTTTATGTTCACGGAATA
TTTATTGTCGTTCCACTTTCTGTAACGGGCATAATCTGCCGCTATCCTCCAGCCGC
CGAAGTCGTAGCCGACCGACACCCTGGGGTGGATGGAATGCGTACGGATGTTTC
TGAAATAATCGCTTACCGTGCTTATTTTGCCTTTTTTTGCACCGGTTGGTTCCGGA
TAATCGTGGGTAATGCGTTCGGCGGCGTAGGCTAAATCCGCCTGCAC

### 18. Ngo1513: Nc_002946.2:1481445-1482281

DNA (+ strand): SEQ ID NO: 282

ATGAATCCAGCCCGCAAAAAACCTTCTCTTCTCTTCTCTTCTCTTCTCTTCTCTTCT
CTTCTCTTCTCTTCTCTTCTCTTCTCTTCGGCAGCGCAGGCGGCAAGTGAAGGCAA
TGGCCGCGGCCCGTATGTGCAGGCGGATTTAGCCTACGCCGCCGAACGCATTAC
CCACGATTATCCGGAACCAACCGCTCCAGGCAAAAACAAAATAAGCACGGTAAG
CGATTATTTCAGAAACATCCGTACGCATTCCATCCACCCCAGGGTGTCGGTCGGC
TACGACTTCGGCGGCTGGCGCATCGCCGCGGATTATGCCCGTTACAGGAAATGG
CACAACAATAAATATTCCGTGAACATAAAAGAGTTGGAAAGAAAGAATAATAA
AACTTTTGGCGGCAACCAGCTTAACATAAAATACCAAAAGACGGAACATCAGGA
AAACGGCACATTCCACGCCGTTTCTTCTCTCGGCTTGTCCGCCGTTTACGATTTCA
AACTCAACGACAAATTCAAACCCTATATCGGTGCGCGCGTCGCCTACGGACACG
TCAGACACAGCATCGATTCGACTAAAAAAATAACAGGTACTCTTACCGCCTACC
CTAGTGATGCTGACGCAGCAGTTACGGTTTATCCTGACGGACATCCGCAAAAAA
ACACCTATCAAAAAAGCAACAGCAGCCGCCGCTTGGGCTTCGGCGCGATGGCGG
GCGTGGGCATAGACGTCGCGCCCGGCCTGACCTTGGACGCCGGCTACCGCTACC
ACAACTGGGGACGCTTGGAAAACACCCGCTTCAAAACCCACGAAGCCTCGTTGG
GCATGCGCTACCGCTTCTGA

RNA: SEQ ID NO: 283

AUGAAUCCAGCCCGCAAAAAACCUUCUCUUCUCUUCUCUUCUCUUCUCUUCUC
UUCUCUUCUCUUCUCUUCUCUUCUCUUCUCUUCGGCAGCGCAGGCGGCAAGUG
AAGGCAAUGGCCGCGGCCCGUAUGUGCAGGCGGAUUUAGCCUACGCCGCCGAA
CGCAUUACCCACGAUUAUCCGGAACCAACCGCUCCAGGCAAAAACAAAAUAAG
CACGGUAAGCGAUUAUUUCAGAAACAUCCGUACGCAUUCCAUCCACCCCAGGG
UGUCGGUCGGCUACGACUUCGGCGGCUGGCGCAUCGCCGCGGAUUAUGCCCGU
UACAGGAAAUGGCACAACAAUAAAUAUUCCGUGAACAUAAAAGAGUUGGAAA
GAAAGAAUAAUAAAACUUUUGGCGGCAACCAGCUUAACAUAAAAUACCAAAA
GACGGAACAUCAGGAAAACGGCACAUUCCACGCCGUUUCUUCUCUCGGCUUGU
CCGCCGUUUACGAUUUCAAACUCAACGACAAAUUCAAACCCUAUAUCGGUGCG
CGCGUCGCCUACGGACACGUCAGACACAGCAUCGAUUCGACUAAAAAAAUAAC
AGGUACUCUUACCGCCUACCCUAGUGAUGCUGACGCAGCAGUUACGGUUUAUC
CUGACGGACAUCCGCAAAAAAACACCUAUCAAAAAAGCAACAGCAGCCGCCGC
UUGGGCUUCGGCGCGAUGGCGGGCGUGGGCAUAGACGUCGCGCCCGGCCUGAC
CUUGGACGCCGGCUACCGCUACCACAACUGGGGACGCUUGGAAAACACCCGCU
UCAAAACCCACGAAGCCUCGUUGGGCAUGCGCUACCGCUUCUGA

cDNA: SEQ ID NO: 284

TCAGAAGCGGTAGCGCATGCCCAACGAGGCTTCGTGGGTTTTGAAGCGGGTGTT
TTCCAAGCGTCCCCAGTTGTGGTAGCGGTAGCCGGCGTCCAAGGTCAGGCCGGG
CGCGACGTCTATGCCCACGCCCGCCATCGCGCCGAAGCCCAAGCGGCGGCTGCT
GTTGCTTTTTTGATAGGTGTTTTTTTGCGGATGTCCGTCAGGATAAACCGTAACTG
CTGCGTCAGCATCACTAGGGTAGGCGGTAAGAGTACCTGTTATTTTTTTAGTCGA
ATCGATGCTGTGTCTGACGTGTCCGTAGGCGACGCGCGCACCGATATAGGGTTTG
AATTTGTCGTTGAGTTTGAAATCGTAAACGGCGGACAAGCCGAGAGAAGAAACG
GCGTGGAATGTGCCGTTTTCCTGATGTTCCGTCTTTTGGTATTTTATGTTAAGCTG
GTTGCCGCCAAAAGTTTTATTATTCTTTCTTTCCAACTCTTTTATGTTCACGGAAT
ATTTATTGTTGTGCCATTTCCTGTAACGGGCATAATCCGCGGCGATGCGCCAGCC
GCCGAAGTCGTAGCCGACCGACACCCTGGGGTGGATGGAATGCGTACGGATGTT
TCTGAAATAATCGCTTACCGTGCTTATTTTGTTTTTGCCTGGAGCGGTTGGTTCCG
GATAATCGTGGGTAATGCGTTCGGCGGCGTAGGCTAAATCCGCCTGCACATACG
GGCCGCGGCCATTGCCTTCACTTGCCGCCTGCGCTGCCGAAGAGAAGAGAAGAG
AAGAGAAGAGAAGAGAAGAGAAGAGAAGAGAAGAGAAGAGAAGAGAAGGTTT
TTTGCGGGCTGGATTCAT

### 19. NGO1553a: NC_002946.2:1531422-1532120

DNA (+ strand): SEQ ID NO: 285

GTGCAGGCGGATTTAGCCTACGCCGCCGAACGCATTACCCACGATTATCCGGAA
CCAACCGGTGCAAAAAAAGACAAAAAAATAAGCACGGTAAGCGATTATTTCAG
AAACATCCGTACGCATTCCGTCCACCCCAGGGTGTCGGTCGGCTACGATTTCGGC
AGCTGGAGGATAGCGGCAGATTATGCCCGTTACAGAAAGTGGAACAACAGTAAA
TATTCCGTCAACATAAAAAGGGTGAAAGAAAACAATGGCAGCGGGAAAAAACT
GACGCAAGACCTGAAGACGGAAAATCAGGAAAACGGTACGTTCCACGCCGTTTC
TTCTCTCGGCTTGTCCGCCGTTTACGATTTCGATACCGGTTCCCGCTTCAAACCCT
ATGCAGGCGTGCGCGTCAGCTACGGACACGTCAGACACAGCATCGATTCGACCA
AAAAAACAACAGATGTTATTACCGCCCCCCCCACTACTTCTGACGGAGCACCTA
CAACTTATAATGCTAATCCACAGACGCAAAACCCTTATCACCAAAGCGACAGCA
TCCGCCGCGTGGGCCTCGGCGTCATCGCCGGCGTCGGTTTCGACATCACGCCCAA
CCTGACCCTGGACACCGGCTACCGCTACCACAACTGGGGACGCCTGGAAAACAC
CCGCTTCAAAACCCACGAAGCCTCATTGGGCATGCGCTACCGCTTCTGA

RNA: SEQ ID NO: 286

GUGCAGGCGGAUUUAGCCUACGCCGCCGAACGCAUUACCCACGAUUAUCCGGA
ACCAACCGGUGCAAAAAAAGACAAAAAAAUAAGCACGGUAAGCGAUUAUUUC
AGAAACAUCCGUACGCAUUCCGUCCACCCCAGGGUGUCGGUCGGCUACGAUUU
CGGCAGCUGGAGGAUAGCGGCAGAUUAUGCCCGUUACAGAAAGUGGAACAAC
AGUAAAUAUUCCGUCAACAUAAAAAGGGUGAAAGAAAACAAUGGCAGCGGGA
AAAAACUGACGCAAGACCUGAAGACGGAAAAUCAGGAAAACGGUACGUUCCA
CGCCGUUUCUUCUCUCGGCUUGUCCGCCGUUUACGAUUUCGAUACCGGUUCCC
GCUUCAAACCCUAUGCAGGCGUGCGCGUCAGCUACGGACACGUCAGACACAGC
AUCGAUUCGACCAAAAAAACAACAGAUGUUAUUACCGCCCCCCCCACUACUUC
UGACGGAGCACCUACAACUUAUAAUGCUAAUCCACAGACGCAAAACCCUUAUC
ACCAAAGCGACAGCAUCCGCCGCGUGGGCCUCGGCGUCAUCGCCGGCGUCGGU
UUCGACAUCACGCCCAACCUGACCCUGGACACCGGCUACCGCUACCACAACUG
GGGACGCCUGGAAAACACCCGCUUCAAAACCCACGAAGCCUCAUUGGGCAUGC
GCUACCGCUUCUGA

cDNA: SEQ ID NO: 287

TCAGAAGCGGTAGCGCATGCCCAATGAGGCTTCGTGGGTTTTGAAGCGGGTGTTT
TCCAGGCGTCCCCAGTTGTGGTAGCGGTAGCCGGTGTCCAGGGTCAGGTTGGGC
GTGATGTCGAAACCGACGCCGGCGATGACGCCGAGGCCCACGCGGCGGATGCTG
TCGCTTTGGTGATAAGGGTTTTGCGTCTGTGGATTAGCATTATAAGTTGTAGGTG
CTCCGTCAGAAGTAGTGGGGGGGGCGGTAATAACATCTGTTGTTTTTTTGGTCGA
ATCGATGCTGTGTCTGACGTGTCCGTAGCTGACGCGCACGCCTGCATAGGGTTTG
AAGCGGGAACCGGTATCGAAATCGTAAACGGCGGACAAGCCGAGAGAAGAAAC
GGCGTGGAACGTACCGTTTTCCTGATTTTCCGTCTTCAGGTCTTGCGTCAGTTTTT
TCCCGCTGCCATTGTTTTCTTTCACCCTTTTTATGTTGACGGAATATTTACTGTTGT
TCCACTTTCTGTAACGGGCATAATCTGCCGCTATCCTCCAGCTGCCGAAATCGTA
GCCGACCGACACCCTGGGGTGGACGGAATGCGTACGGATGTTTCTGAAATAATC
GCTTACCGTGCTTATTTTTTTGTCTTTTTTTGCACCGGTTGGTTCCGGATAATCGT
GGGTAATGCGTTCGGCGGCGTAGGCTAAATCCGCCTGCAC

### 20. Ngo1762: Nc_002946.2:1724401-1724637

DNA (+ strand): SEQ ID NO: 288

ATGTCAAACATCGAACAACAAGTTAAGAAAATTATTGCTGAACAACTGGGCGTA
AACGAAGCCGACGTGAAAAACGAATCTTCCTTCCAAGACGACTTGGGCGCGGAT
TCTTTGGATACCGTGGAGTTGGTTATGGCTTTGGAAGAAGCCTTCGGCTGCGAAA
TCCCCGACGAAGATGCCGAAAAAATCACCACCGTCCAACTGGCTATCGACTACA
TCAATGCCCACAACGGCTAA

RNA: SEQ ID NO: 289

AUGUCAAACAUCGAACAACAAGUUAAGAAAAUUAUUGCUGAACAACUGGGCG
UAAACGAAGCCGACGUGAAAAACGAAUCUUCCUUCCAAGACGACUUGGGCGC
GGAUUCUUUGGAUACCGUGGAGUUGGUUAUGGCUUUGGAAGAAGCCUUCGGC
UGCGAAAUCCCCGACGAAGAUGCCGAAAAAAUCACCACCGUCCAACUGGCUAU
CGACUACAUCAAUGCCCACAACGGCUAA

cDNA: SEQ ID NO: 290

TTAGCCGTTGTGGGCATTGATGTAGTCGATAGCCAGTTGGACGGTGGTGATTTTT
TCGGCATCTTCGTCGGGGATTTCGCAGCCGAAGGCTTCTTCCAAAGCCATAACCA
ACTCCACGGTATCCAAAGAATCCGCGCCCAAGTCGTCTTGGAAGGAAGATTCGT
TTTTCACGTCGGCTTCGTTTACGCCCAGTTGTTCAGCAATAATTTTCTTAACTTGT
TGTTCGATGTTTGACAT

### 21. NGO1842: NC_002946.2:c1808872-1807688

DNA (- strand): SEQ ID NO: 291

ATGGCTAAGGAAAAATTCGAACGTAGCAAACCGCACGTAAACGTTGGCACCATC
GGTCACGTTGACCATGGTAAAACCACCCTGACTGCTGCTTTGACTACTATTTTAG
CTAAAAAATTCGGCGGCGCTGCAAAAGCTTACGACCAAATCGACAACGCACCCG
AAGAAAAAGCACGCGGTATTACCATTAACACCTCGCACGTAGAATACGAAACCG
AAACCCGCCACTACGCACACGTAGACTGTCCGGGTCACGCCGACTACGTTAAAA
ACATGATTACCGGCGCCGCACAAATGGACGGTGCAATCCTGGTATGTTCTGCTGC
CGACGGCCCTATGCCGCAAACCCGCGAACACATCCTGCTGGCCCGTCAAGTAGG
CGTACCTTACATCATCGTGTTCATGAACAAATGCGACATGGTCGACGATGCCGAG
CTGTTGGAACTGGTTGAAATGGAAATCCGCGACCTGCTGTCCAGCTACGACTTCC
CCGGCGACGACTGCCCGATCGTACAAGGTTCCGCACTGAAAGCCTTGGAAGGCG
ATGCCGCTTACGAAGAAAAAATCTTCGAACTGGCTACCGCATTGGACAGCTACA
TCCCGACTCCCGAGCGTGCCGTGGACAAACCATTCCTGCTGCCTATCGAAGACGT
GTTCTCCATTTCCGGCCGCGGTACCGTAGTCACCGGCCGTGTAGAGCGAGGTATC
ATCCACGTTGGTGACGAGATTGAAATCGTCGGTCTGAAAGAAACCCAAAAAACC
ACCTGTACCGGCGTTGAAATGTTCCGCAAACTGCTGGACGAAGGTCAGGCGGGC
GACAACGTAGGCGTATTGCTGCGCGGTACCAAACGTGAAGACGTAGAACGCGGT
CAGGTATTGGCCAAACCGGGTACTATCACTCCTCACACCAAGTTCAAAGCAGAA
GTGTACGTATTGAGCAAAGAAGAGGGCGGCCGCCATACCCCGTTTTTCGCCAAC
TACCGTCCCCAATTCTACTTCCGTACCACTGACGTAACCGGCGCGGTTACTTTGG
AAAAAGGTGTGGAAATGGTAATGCCGGGTGAGAACGTAACCATTACTGTAGAAC
TGATTGCGCCTATCGCTATGGAAGAAGGTCTGCGCTTTGCGATTCGCGAAGGCGG
CCGTACCGTGGGTGCCGGCGTGGTTTCTTCTGTTATCGCTTAA

RNA: SEQ ID NO: 292

AUGGCUAAGGAAAAAUUCGAACGUAGCAAACCGCACGUAAACGUUGGCACCA
UCGGUCACGUUGACCAUGGUAAAACCACCCUGACUGCUGCUUUGACUACUAUU
UUAGCUAAAAAAUUCGGCGGCGCUGCAAAAGCUUACGACCAAAUCGACAACG
CACCCGAAGAAAAAGCACGCGGUAUUACCAUUAACACCUCGCACGUAGAAUAC
GAAACCGAAACCCGCCACUACGCACACGUAGACUGUCCGGGUCACGCCGACUA
CGUUAAAAACAUGAUUACCGGCGCCGCACAAAUGGACGGUGCAAUCCUGGUA
UGUUCUGCUGCCGACGGCCCUAUGCCGCAAACCCGCGAACACAUCCUGCUGGC
CCGUCAAGUAGGCGUACCUUACAUCAUCGUGUUCAUGAACAAAUGCGACAUG
GUCGACGAUGCCGAGCUGUUGGAACUGGUUGAAAUGGAAAUCCGCGACCUGC
UGUCCAGCUACGACUUCCCCGGCGACGACUGCCCGAUCGUACAAGGUUCCGCA
CUGAAAGCCUUGGAAGGCGAUGCCGCUUACGAAGAAAAAAUCUUCGAACUGG
CUACCGCAUUGGACAGCUACAUCCCGACUCCCGAGCGUGCCGUGGACAAACCA
UUCCUGCUGCCUAUCGAAGACGUGUUCUCCAUUUCCGGCCGCGGUACCGUAGU
CACCGGCCGUGUAGAGCGAGGUAUCAUCCACGUUGGUGACGAGAUUGAAAUC
GUCGGUCUGAAAGAAACCCAAAAAACCACCUGUACCGGCGUUGAAAUGUUCC
GCAAACUGCUGGACGAAGGUCAGGCGGGCGACAACGUAGGCGUAUUGCUGCG
CGGUACCAAACGUGAAGACGUAGAACGCGGUCAGGUAUUGGCCAAACCGGGU
ACUAUCACUCCUCACACCAAGUUCAAAGCAGAAGUGUACGUAUUGAGCAAAG
AAGAGGGCGGCCGCCAUACCCCGUUUUUCGCCAACUACCGUCCCCAAUUCUAC
UUCCGUACCACUGACGUAACCGGCGCGGUUACUUUGGAAAAAGGUGUGGAAA
UGGUAAUGCCGGGUGAGAACGUAACCAUUACUGUAGAACUGAUUGCGCCUAU
CGCUAUGGAAGAAGGUCUGCGCUUUGCGAUUCGCGAAGGCGGCCGUACCGUG
GGUGCCGGCGUGGUUUCUUCUGUUAUCGCUUAA

cDNA: SEQ ID NO: 293

TTAAGCGATAACAGAAGAAACCACGCCGGCACCCACGGTACGGCCGCCTTCGCG
AATCGCAAAGCGCAGACCTTCTTCCATAGCGATAGGCGCAATCAGTTCTACAGT
AATGGTTACGTTCTCACCCGGCATTACCATTTCCACACCTTTTTCCAAAGTAACC
GCGCCGGTTACGTCAGTGGTACGGAAGTAGAATTGGGGACGGTAGTTGGCGAAA
AACGGGGTATGGCGGCCGCCCTCTTCTTTGCTCAATACGTACACTTCTGCTTTGA
ACTTGGTGTGAGGAGTGATAGTACCCGGTTTGGCCAATACCTGACCGCGTTCTAC
GTCTTCACGTTTGGTACCGCGCAGCAATACGCCTACGTTGTCGCCCGCCTGACCT
TCGTCCAGCAGTTTGCGGAACATTTCAACGCCGGTACAGGTGGTTTTTTGGGTTT
CTTTCAGACCGACGATTTCAATCTCGTCACCAACGTGGATGATACCTCGCTCTAC
ACGGCCGGTGACTACGGTACCGCGGCCGGAAATGGAGAACACGTCTTCGATAGG
CAGCAGGAATGGTTTGTCCACGGCACGCTCGGGAGTCGGGATGTAGCTGTCCAA
TGCGGTAGCCAGTTCGAAGATTTTTTCTTCGTAAGCGGCATCGCCTTCCAAGGCT
TTCAGTGCGGAACCTTGTACGATCGGGCAGTCGTCGCCGGGGAAGTCGTAGCTG
GACAGCAGGTCGCGGATTTCCATTTCAACCAGTTCCAACAGCTCGGCATCGTCGA
CCATGTCGCATTTGTTCATGAACACGATGATGTAAGGTACGCCTACTTGACGGGC
CAGCAGGATGTGTTCGCGGGTTTGCGGCATAGGGCCGTCGGCAGCAGAACATAC
CAGGATTGCACCGTCCATTTGTGCGGCGCCGGTAATCATGTTTTTAACGTAGTCG
GCGTGACCCGGACAGTCTACGTGTGCGTAGTGGCGGGTTTCGGTTTCGTATTCTA
CGTGCGAGGTGTTAATGGTAATACCGCGTGCTTTTTCTTCGGGTGCGTTGTCGAT
TTGGTCGTAAGCTTTTGCAGCGCCGCCGAATTTTTTAGCTAAAATAGTAGTCAAA
GCAGCAGTCAGGGTGGTTTTACCATGGTCAACGTGACCGATGGTGCCAACGTTTA
CGTGCGGTTTGCTACGTTCGAATTTTTCCTTAGCCAT

### 22. NGO1871: NC_002946.2:c1842986-1842483

DNA (- strand): SEQ ID NO: 294

ATGGCTTTACTGAATATCTTGCAATATCCCGACGAGCGTCTGCACACGGTGGCAA
AGCCTGTCGAACAAGTTGACGAGCGCATCCGGAAGCTGGTTGCCGATATGTTTG
AAACGATGTACGAATCGCGCGGCATCGGGCTGGCGGCGACGCAGGTCGATGTGC
ACGAACGCGTGGTCGTGATGGATTTGACCGAAGACCGCAGCGAACCGCGCGTGT
TCATCAACCCCGTCATCGTTGAAAAAGACGGCGAAACCACTTACGAAGAGGGCT
GCCTGTCCGTACCGGGCATTTACGACGCCGTTACCCGCGCCGAACGCGTCAAGG
TCGAGGCTTTGAACGAAAAAGGCGAAAAATTCACGCTGGAGGCGGACGGGCTGC
TGGCGATTTGCGTGCAGCACGAGTTAGATCACCTGATGGGCATCGTGTTTGTCGA
ACGCCTTTCCCAACTCAAGCAGGGGCGGATTAAGACCAAACTGAAAAAACGTCA
GAAACATACGATTTGA

RNA: SEQ ID NO: 295

AUGGCUUUACUGAAUAUCUUGCAAUAUCCCGACGAGCGUCUGCACACGGUGG
CAAAGCCUGUCGAACAAGUUGACGAGCGCAUCCGGAAGCUGGUUGCCGAUAU
GUUUGAAACGAUGUACGAAUCGCGCGGCAUCGGGCUGGCGGCGACGCAGGUC
GAUGUGCACGAACGCGUGGUCGUGAUGGAUUUGACCGAAGACCGCAGCGAAC
CGCGCGUGUUCAUCAACCCCGUCAUCGUUGAAAAAGACGGCGAAACCACUUAC
GAAGAGGGCUGCCUGUCCGUACCGGGCAUUUACGACGCCGUUACCCGCGCCGA
ACGCGUCAAGGUCGAGGCUUUGAACGAAAAAGGCGAAAAAUUCACGCUGGAG
GCGGACGGGCUGCUGGCGAUUUGCGUGCAGCACGAGUUAGAUCACCUGAUGG
GCAUCGUGUUUGUCGAACGCCUUUCCCAACUCAAGCAGGGGCGGAUUAAGACC
AAACUGAAAAAACGUCAGAAACAUACGAUUUGA

cDNA: SEQ ID NO: 296

TCAAATCGTATGTTTCTGACGTTTTTTCAGTTTGGTCTTAATCCGCCCCTGCTTGA
GTTGGGAAAGGCGTTCGACAAACACGATGCCCATCAGGTGATCTAACTCGTGCT
GCACGCAAATCGCCAGCAGCCCGTCCGCCTCCAGCGTGAATTTTTCGCCTTTTTC
GTTCAAAGCCTCGACCTTGACGCGTTCGGCGCGGGTAACGGCGTCGTAAATGCC
CGGTACGGACAGGCAGCCCTCTTCGTAAGTGGTTTCGCCGTCTTTTTCAACGATG
ACGGGGTTGATGAACACGCGCGGTTCGCTGCGGTCTTCGGTCAAATCCATCACG
ACCACGCGTTCGTGCACATCGACCTGCGTCGCCGCCAGCCCGATGCCGCGCGATT
CGTACATCGTTTCAAACATATCGGCAACCAGCTTCCGGATGCGCTCGTCAACTTG
TTCGACAGGCTTTGCCACCGTGTGCAGACGCTCGTCGGGATATTGCAAGATATTC
AGTAAAGCCAT

### 23. Ngo1908: Nc_002946.2:1881198-1882241

DNA (+ strand): SEQ ID NO: 297

ATGCAGATTACCGACTTACTCGCCTTCGGCGCTAAAAACAAAGCATCCGACCTTC
ACCTGAGTTCGGGCATATCCCCTATGATTCGGGTTCACGGCGACATGCGGCGCAT
CAACCTTCCCGAAATGAGCGCGGAAGAGGTCGGCAATATGGTAACTTCGGTGAT
GAACGACCACCAGCGGAAAATCTACCAGCAAAACTTGGAAGTCGACTTCTCGTT
CGAACTGCCCAACGTCGCCCGATTCCGCGTCAACGCCTTCAACACCGGCCGCGG
CCCCGCCGCCGTATTCCGCACCATTCCCAGCACCGTCTTATCGCTGGAAGAATTG
AAAGCCCCGAGCATTTTCCAAAAAATCGCAGAATCGCCGCGCGGCATGGTATTG
GTTACCGGCCCTACCGGTTCGGGCAAATCGACCACGCTTGCCGCGATGATCAACT
ACATCAACGAAACCCAGCCGGCACACATCCTGACCATCGAAGACCCGATCGAAT
TCGTCCACCAAAGCAAAAAATCCCTGATTAACCAACGCGAGCTGCACCAGCACA
CCCTCAGCTTCGCCAACGCGCTGAGTTCCGCATTGCGCGAAGACCCCGACGTTAT
CCTTGTCGGCGAGATGCGCGACCCCGAAACCATCGGCTTGGCACTGACCGCCGC
CGAAACCGGACACTTGGTTTTCGGCACGCTGCACACGACCGGCGCGGCAAAAAC
CGTCGACCGTATCGTGGACGTATTCCCCGCCGGAGAGAAAGAAATGGTGCGTTC
CATGCTGTCCGAATCGCTGACCGCCGTCATCTCCCAAAACCTGCTGAAAACGCAC
GACGGCGACGGCCGTGTCGCCTCGCACGAAATCCTGATTGCCAACCCCGCCGTC
CGCAACCTCATCCGCGAAAACAAAATCACGCAGATTAACTCCGTCCTGCAAACC
GGGCGGGCGAGCGGTATGCAGACGATGGACCAATCGCTGCAATCGCTGGTGCGC
CAAGGGCTGATCGCACCGGAAGCCACACGCAGACGCGCGCAAAACAGCGAAAG
TATGAGTTTCTGA

RNA: SEQ ID NO: 298

AUGCAGAUUACCGACUUACUCGCCUUCGGCGCUAAAAACAAAGCAUCCGACCU
UCACCUGAGUUCGGGCAUAUCCCCUAUGAUUCGGGUUCACGGCGACAUGCGGC
GCAUCAACCUUCCCGAAAUGAGCGCGGAAGAGGUCGGCAAUAUGGUAACUUC
GGUGAUGAACGACCACCAGCGGAAAAUCUACCAGCAAAACUUGGAAGUCGAC
UUCUCGUUCGAACUGCCCAACGUCGCCCGAUUCCGCGUCAACGCCUUCAACAC
CGGCCGCGGCCCCGCCGCCGUAUUCCGCACCAUUCCCAGCACCGUCUUAUCGC
UGGAAGAAUUGAAAGCCCCGAGCAUUUUCCAAAAAAUCGCAGAAUCGCCGCG
CGGCAUGGUAUUGGUUACCGGCCCUACCGGUUCGGGCAAAUCGACCACGCUUG
CCGCGAUGAUCAACUACAUCAACGAAACCCAGCCGGCACACAUCCUGACCAUC
GAAGACCCGAUCGAAUUCGUCCACCAAAGCAAAAAAUCCCUGAUUAACCAACG
CGAGCUGCACCAGCACACCCUCAGCUUCGCCAACGCGCUGAGUUCCGCAUUGC
GCGAAGACCCCGACGUUAUCCUUGUCGGCGAGAUGCGCGACCCCGAAACCAUC
GGCUUGGCACUGACCGCCGCCGAAACCGGACACUUGGUUUUCGGCACGCUGCA
CACGACCGGCGCGGCAAAAACCGUCGACCGUAUCGUGGACGUAUUCCCCGCCG
GAGAGAAAGAAAUGGUGCGUUCCAUGCUGUCCGAAUCGCUGACCGCCGUCAU
CUCCCAAAACCUGCUGAAAACGCACGACGGCGACGGCCGUGUCGCCUCGCACG
AAAUCCUGAUUGCCAACCCCGCCGUCCGCAACCUCAUCCGCGAAAACAAAAUC
ACGCAGAUUAACUCCGUCCUGCAAACCGGGCGGGCGAGCGGUAUGCAGACGAU
GGACCAAUCGCUGCAAUCGCUGGUGCGCCAAGGGCUGAUCGCACCGGAAGCCA
CACGCAGACGCGCGCAAAACAGCGAAAGUAUGAGUUUCUGA

cDNA: SEQ ID NO: 299

TCAGAAACTCATACTTTCGCTGTTTTGCGCGCGTCTGCGTGTGGCTTCCGGTGCG
ATCAGCCCTTGGCGCACCAGCGATTGCAGCGATTGGTCCATCGTCTGCATACCGC
TCGCCCGCCCGGTTTGCAGGACGGAGTTAATCTGCGTGATTTTGTTTTCGCGGAT
GAGGTTGCGGACGGCGGGGTTGGCAATCAGGATTTCGTGCGAGGCGACACGGCC
GTCGCCGTCGTGCGTTTTCAGCAGGTTTTGGGAGATGACGGCGGTCAGCGATTCG
GACAGCATGGAACGCACCATTTCTTTCTCTCCGGCGGGGAATACGTCCACGATAC
GGTCGACGGTTTTTGCCGCGCCGGTCGTGTGCAGCGTGCCGAAAACCAAGTGTC
CGGTTTCGGCGGCGGTCAGTGCCAAGCCGATGGTTTCGGGGTCGCGCATCTCGCC
GACAAGGATAACGTCGGGGTCTTCGCGCAATGCGGAACTCAGCGCGTTGGCGAA
GCTGAGGGTGTGCTGGTGCAGCTCGCGTTGGTTAATCAGGGATTTTTTGCTTTGG
TGGACGAATTCGATCGGGTCTTCGATGGTCAGGATGTGTGCCGGCTGGGTTTCGT
TGATGTAGTTGATCATCGCGGCAAGCGTGGTCGATTTGCCCGAACCGGTAGGGC
CGGTAACCAATACCATGCCGCGCGGCGATTCTGCGATTTTTTGGAAAATGCTCGG
GGCTTTCAATTCTTCCAGCGATAAGACGGTGCTGGGAATGGTGCGGAATACGGC
GGCGGGGCCGCGGCCGGTGTTGAAGGCGTTGACGCGGAATCGGGCGACGTTGGG
CAGTTCGAACGAGAAGTCGACTTCCAAGTTTTGCTGGTAGATTTTCCGCTGGTGG
TCGTTCATCACCGAAGTTACCATATTGCCGACCTCTTCCGCGCTCATTTCGGGAA
GGTTGATGCGCCGCATGTCGCCGTGAACCCGAATCATAGGGGATATGCCCGAAC
TCAGGTGAAGGTCGGATGCTTTGTTTTTAGCGCCGAAGGCGAGTAAGTCGGTAAT
CTGCAT

### 24. NGO1982: NC_002946.2:c1957797-1957498

DNA (- strand): SEQ ID NO: 300

ATGAAAATATTTGAAAATATAGAAGATGTTAAAGCCATCCGTAAAAAGACCGGG
ATGAACCAGATAGACTTCTGGGGCAAGGTCGGCGTTACTCAATCCGGAGGTTCA
CGCTACGAAACCGGCCGTAAGATGCCCAAACCCGTACGCGAACTGCTCCGCCTC
GTCCATATCGAATGCCTCGATTTGGCAAAAGTCAACAAAAAAGATATGGAAATC
GCCGCCCTGTTGAAAAAACACCATCCCGACCTGTATGCCGAGTTGTCCAAACAG
ACCAAGTCCGAAAGAAAAAAACAAAGTTAA

RNA: SEQ ID NO: 301

AUGAAAAUAUUUGAAAAUAUAGAAGAUGUUAAAGCCAUCCGUAAAAAGACCG
GGAUGAACCAGAUAGACUUCUGGGGCAAGGUCGGCGUUACUCAAUCCGGAGG
UUCACGCUACGAAACCGGCCGUAAGAUGCCCAAACCCGUACGCGAACUGCUCC
GCCUCGUCCAUAUCGAAUGCCUCGAUUUGGCAAAAGUCAACAAAAAAGAUAU
GGAAAUCGCCGCCCUGUUGAAAAAACACCAUCCCGACCUGUAUGCCGAGUUGU
CCAAACAGACCAAGUCCGAAAGAAAAAAACAAAGUUAA

cDNA: SEQ ID NO: 302

TTAACTTTGTTTTTTTCTTTCGGACTTGGTCTGTTTGGACAACTCGGCATACAGGT
CGGGATGGTGTTTTTTCAACAGGGCGGCGATTTCCATATCTTTTTTGTTGACTTTT
GCCAAATCGAGGCATTCGATATGGACGAGGCGGAGCAGTTCGCGTACGGGTTTG
GGCATCTTACGGCCGGTTTCGTAGCGTGAACCTCCGGATTGAGTAACGCCGACCT
TGCCCCAGAAGTCTATCTGGTTCATCCCGGTCTTTTTACGGATGGCTTTAACATCT
TCTATATTTTCAAATATTTTCAT

### 25. NGO2060a: NC_002946.2:c2037067-2036384

DNA (- strand): SEQ ID NO: 303

GTGCAGGCGGATTTAGCCTACGCCGCCGAACGCATTACCCACGATTATCCGGAA
CCAACCGCTCCAGGCAAAAACAAAATAAGCACGGTAAGCGATTATTTCAGAAAC
ATCCGTACGCATTCCATCCACCCCAGGGTGTCGGTCGGCTACGACTTCGGCGGCT
GGAGGATAGCGGCAGATTATGCCCGTTACAGAAAGTGGAACGACAATAAATATT
CCGTCGACATAAAAGAGTTGGAAAACAAGAATCAGAATAAGAGAGACCTGAAG
ACGGAAAATCAGGAAAACGGCAGCTTCCACGCCGTTTCTTCTCTCGGCTTATCAG
CCGTTTACGATTTCAAACTCAACGACAAATTCAAACCCTATATCGGTGCGCGCGT
CGCCTACGGACACGTCAGACACAGCATCGATTCGACTAAAAAAATAACAGGTAC
TCTTACCGCCTACCCTAGTGATGCTGACGCAGCAGTTACGGTTTATCCTGACGGA
CATCCGCAAAAAAACACCTATCAAAAAAGCAACAGCAGCCGCCGCTTGGGCTTC
GGCGCGATGGCGGGCGTGGGCATAGACGTCGCGCCCGGCCTGACCTTGGACGCC
GGCTACCGCTACCACAACTGGGGACGCTTGGAAAACACCCGCTTCAAAACCCAC
GAAGCCTCATTGGGCATGCGCTACCGCTTCTGA

RNA: SEQ ID NO: 304

GUGCAGGCGGAUUUAGCCUACGCCGCCGAACGCAUUACCCACGAUUAUCCGGA
ACCAACCGCUCCAGGCAAAAACAAAAUAAGCACGGUAAGCGAUUAUUUCAGA
AACAUCCGUACGCAUUCCAUCCACCCCAGGGUGUCGGUCGGCUACGACUUCGG
CGGCUGGAGGAUAGCGGCAGAUUAUGCCCGUUACAGAAAGUGGAACGACAAU
AAAUAUUCCGUCGACAUAAAAGAGUUGGAAAACAAGAAUCAGAAUAAGAGAG
ACCUGAAGACGGAAAAUCAGGAAAACGGCAGCUUCCACGCCGUUUCUUCUCUC
GGCUUAUCAGCCGUUUACGAUUUCAAACUCAACGACAAAUUCAAACCCUAUA
UCGGUGCGCGCGUCGCCUACGGACACGUCAGACACAGCAUCGAUUCGACUAAA
AAAAUAACAGGUACUCUUACCGCCUACCCUAGUGAUGCUGACGCAGCAGUUAC
GGUUUAUCCUGACGGACAUCCGCAAAAAAACACCUAUCAAAAAAGCAACAGC
AGCCGCCGCUUGGGCUUCGGCGCGAUGGCGGGCGUGGGCAUAGACGUCGCGCC
CGGCCUGACCUUGGACGCCGGCUACCGCUACCACAACUGGGGACGCUUGGAAA
ACACCCGCUUCAAAACCCACGAAGCCUCAUUGGGCAUGCGCUACCGCUUCUGA

cDNA: SEQ ID NO: 305

TCAGAAGCGGTAGCGCATGCCCAATGAGGCTTCGTGGGTTTTGAAGCGGGTGTTT
TCCAAGCGTCCCCAGTTGTGGTAGCGGTAGCCGGCGTCCAAGGTCAGGCCGGGC
GCGACGTCTATGCCCACGCCCGCCATCGCGCCGAAGCCCAAGCGGCGGCTGCTG
TTGCTTTTTTGATAGGTGTTTTTTTGCGGATGTCCGTCAGGATAAACCGTAACTGC
TGCGTCAGCATCACTAGGGTAGGCGGTAAGAGTACCTGTTATTTTTTTAGTCGAA
TCGATGCTGTGTCTGACGTGTCCGTAGGCGACGCGCGCACCGATATAGGGTTTGA
ATTTGTCGTTGAGTTTGAAATCGTAAACGGCTGATAAGCCGAGAGAAGAAACGG
CGTGGAAGCTGCCGTTTTCCTGATTTTCCGTCTTCAGGTCTCTCTTATTCTGATTC
TTGTTTTCCAACTCTTTTATGTCGACGGAATATTTATTGTCGTTCCACTTTCTGTA
ACGGGCATAATCTGCCGCTATCCTCCAGCCGCCGAAGTCGTAGCCGACCGACAC
CCTGGGGTGGATGGAATGCGTACGGATGTTTCTGAAATAATCGCTTACCGTGCTT
ATTTTGTTTTTGCCTGGAGCGGTTGGTTCCGGATAATCGTGGGTAATGCGTTCGG
CGGCGTAGGCTAAATCCGCCTGCAC

### 26. Ngo2084: Nc_002946.2:2061613-2062296

DNA (+ strand): SEQ ID NO: 306

ATGCAACACGACGTTTACGACTACACCGCGCATACGGTTTCTAAAAACACCGTC
CTGCAGAAAACCTACCGCCTGCTCGGATTTTCATTCATTCCGGCAGCCGCAGGCG
CGGCACTTGCCGCCAATGCCGGTTTCAATTTTTACGCCGCCTTCGGTTCGCGCTG
GATAGGATTTGCCGTCGTATTGGCGTTTTTCTACGGTATGATCCACTTCATCGAA
AAAAACCGTTACAGCAATACCGGCGTTACCCTGCTGATGGTATTCACATTCGGTA
TGGGCGTATTGATCGGCCCCGTGCTGCAATACGCACTCCATATTGCCGACGGTGC
GAAAATCGTCGGCATTGCCGCCGCGATGACCGCCGCCGTCTTTTTAACGATGTCC
GCATTGGCACGCCGAACCCGGCTCGATATGAACGCGCTCGGACGCTTCCTGACC
GTAGGTGCGGTCATTCTGATGGTCGCCGTGGTTGCCAATCTGTTTTTGGGTATTCC
CGCACTCGCCCTGACCATTTCCGCCGGTTTTGTCTTGTTCAGTTCCTTAATAATTA
TGTGGCAGGTACGCACCGTCATCGACGGCGGCGAAGACAGTTACATCAGCGCGG
CACTGACACTGTTTATCTCGCTTTACAACATCTTCAGCAGCCTGCTCAACATCCT
GCTGTCCTTAAACGGCGACGACTGA

RNA: SEQ ID NO: 307

AUGCAACACGACGUUUACGACUACACCGCGCAUACGGUUUCUAAAAACACCGU
CCUGCAGAAAACCUACCGCCUGCUCGGAUUUUCAUUCAUUCCGGCAGCCGCAG
GCGCGGCACUUGCCGCCAAUGCCGGUUUCAAUUUUUACGCCGCCUUCGGUUCG
CGCUGGAUAGGAUUUGCCGUCGUAUUGGCGUUUUUCUACGGUAUGAUCCACU
UCAUCGAAAAAAACCGUUACAGCAAUACCGGCGUUACCCUGCUGAUGGUAUU
CACAUUCGGUAUGGGCGUAUUGAUCGGCCCCGUGCUGCAAUACGCACUCCAUA
UUGCCGACGGUGCGAAAAUCGUCGGCAUUGCCGCCGCGAUGACCGCCGCCGUC
UUUUUAACGAUGUCCGCAUUGGCACGCCGAACCCGGCUCGAUAUGAACGCGCU
CGGACGCUUCCUGACCGUAGGUGCGGUCAUUCUGAUGGUCGCCGUGGUUGCCA
AUCUGUUUUUGGGUAUUCCCGCACUCGCCCUGACCAUUUCCGCCGGUUUUGUC
UUGUUCAGUUCCUUAAUAAUUAUGUGGCAGGUACGCACCGUCAUCGACGGCG
GCGAAGACAGUUACAUCAGCGCGGCACUGACACUGUUUAUCUCGCUUUACAAC
AUCUUCAGCAGCCUGCUCAACAUCCUGCUGUCCUUAAACGGCGACGACUGA

cDNA: SEQ ID NO: 308

TCAGTCGTCGCCGTTTAAGGACAGCAGGATGTTGAGCAGGCTGCTGAAGATGTT
GTAAAGCGAGATAAACAGTGTCAGTGCCGCGCTGATGTAACTGTCTTCGCCGCC
GTCGATGACGGTGCGTACCTGCCACATAATTATTAAGGAACTGAACAAGACAAA
ACCGGCGGAAATGGTCAGGGCGAGTGCGGGAATACCCAAAAACAGATTGGCAA
CCACGGCGACCATCAGAATGACCGCACCTACGGTCAGGAAGCGTCCGAGCGCGT
TCATATCGAGCCGGGTTCGGCGTGCCAATGCGGACATCGTTAAAAAGACGGCGG
CGGTCATCGCGGCGGCAATGCCGACGATTTTCGCACCGTCGGCAATATGGAGTG
CGTATTGCAGCACGGGGCCGATCAATACGCCCATACCGAATGTGAATACCATCA
GCAGGGTAACGCCGGTATTGCTGTAACGGTTTTTTTCGATGAAGTGGATCATACC
GTAGAAAAACGCCAATACGACGGCAAATCCTATCCAGCGCGAACCGAAGGCGG
CGTAAAAATTGAAACCGGCATTGGCGGCAAGTGCCGCGCCTGCGGCTGCCGGAA
TGAATGAAAATCCGAGCAGGCGGTAGGTTTTCTGCAGGACGGTGTTTTTAGAAA
CCGTATGCGCGGTGTAGTCGTAAACGTCGTGTTGCAT

### 27. NGO2134: NC_002946.2:c2114153-2113941

DNA (- strand): SEQ ID NO: 309

ATGCCTGCAATCCGCGTAAAAGAGAATGAACCATTTGAAGTCGCTATGCGCCGT
TTCAAACGCGCCGTAGAAAAAACCGGCCTGCTGACCGAGCTGCGCGCCCGCGAA
GCCTACGAAAAACCGACTACCGAACGCAAACGCAAAAAAGCGGCAGCCGTAAA
ACGCCTGCAAAAACGCCTGCGCAGCCAACAGCTGCCGCCCAAAATGTACTAA

RNA: SEQ ID NO: 310

AUGCCUGCAAUCCGCGUAAAAGAGAAUGAACCAUUUGAAGUCGCUAUGCGCC
GUUUCAAACGCGCCGUAGAAAAAACCGGCCUGCUGACCGAGCUGCGCGCCCGC
GAAGCCUACGAAAAACCGACUACCGAACGCAAACGCAAAAAAGCGGCAGCCGU
AAAACGCCUGCAAAAACGCCUGCGCAGCCAACAGCUGCCGCCCAAAAUGUACU
AA

cDNA: SEQ ID NO: 311

TTAGTACATTTTGGGCGGCAGCTGTTGGCTGCGCAGGCGTTTTTGCAGGCGTTTT
ACGGCTGCCGCTTTTTTGCGTTTGCGTTCGGTAGTCGGTTTTTCGTAGGCTTCGCG
GGCGCGCAGCTCGGTCAGCAGGCCGGTTTTTTCTACGGCGCGTTTGAAACGGCG
CATAGCGACTTCAAATGGTTCATTCTCTTTTACGCGGATTGCAGGCAT

### 28. Ngo2145: Nc_002946.2:2122709-2122945

DNA (+ strand): SEQ ID NO: 312

ATGGGTTTGATTGCTATCGCATGTGGTTTGATCGTTGCATTGGGTGCATTGGGTG
CATCTATCGGTATCGCAATGGTCGGTTCTAAATATTTGGAGTCTTCTGCTCGCCA
ACCTGAACTGATTGGTCCGCTGCAAACCAAACTGTTCCTGATTGCCGGTCTGATT
GATGCCGCATTCTTGATCGGTGTCGCCATTGCACTACTGTTCGCCTTCGTCAACC
CGTTTGCAGGTGCATAA

RNA: SEQ ID NO: 313

AUGGGUUUGAUUGCUAUCGCAUGUGGUUUGAUCGUUGCAUUGGGUGCAUUGG
GUGCAUCUAUCGGUAUCGCAAUGGUCGGUUCUAAAUAUUUGGAGUCUUCUGC
UCGCCAACCUGAACUGAUUGGUCCGCUGCAAACCAAACUGUUCCUGAUUGCCG
GUCUGAUUGAUGCCGCAUUCUUGAUCGGUGUCGCCAUUGCACUACUGUUCGCC
UUCGUCAACCCGUUUGCAGGUGCAUAA

cDNA: SEQ ID NO: 314

TTATGCACCTGCAAACGGGTTGACGAAGGCGAACAGTAGTGCAATGGCGACACC
GATCAAGAATGCGGCATCAATCAGACCGGCAATCAGGAACAGTTTGGTTTGCAG
CGGACCAATCAGTTCAGGTTGGCGAGCAGAAGACTCCAAATATTTAGAACCGAC
CATTGCGATACCGATAGATGCACCCAATGCACCCAATGCAACGATCAAACCACA
TGCGATAGCAATCAAACCCAT

### 29. Ngo2146: Nc_002946.2:2123015-2123485

DNA (+ strand): SEQ ID NO: 315

GTGAATATCAATGCAACATTATTCGCTCAAATCATCGTCTTTTTCGGTTTGGTATG
GTTTACCATGAAATTTGTGTGGCCGCCGATTGCAAAAGCTTTGGATGAGCGTGCC
GCAAAAATCGCCGAGGGCTTGGCTGCCGCCGAGCGTGGTAAAAGCGATTTCGAG
CAGGCTGAAAAAAAGGTTGCAGAACTTTTGGCAGAAGGGCGTAATCAGGTTTCC
GAAATGGTTGCCAACGCCGAAAAACGTGCCGCCAAAATTGTCGAAGAAGCCAA
AGAACAGGCTTCTTCCGAGGCGGCGCGCATTGCAGCTCAGGCAAAGGCCGATGT
GGAGCAGGAATTGTTCCGCGCACGCGAATCCCTGCGCGATCAGGTTGCCGTGTT
GGCTGTCAAAGGTGCCGAATCTATTTTGCGCAGCGAAGTCGATGCTTCCAAACAC
GCAAAACTGCTCGATACCCTGAAACAGGAGTTGTAA

RNA: SEQ ID NO: 316

GUGAAUAUCAAUGCAACAUUAUUCGCUCAAAUCAUCGUCUUUUUCGGUUUGG
UAUGGUUUACCAUGAAAUUUGUGUGGCCGCCGAUUGCAAAAGCUUUGGAUGA
GCGUGCCGCAAAAAUCGCCGAGGGCUUGGCUGCCGCCGAGCGUGGUAAAAGCG
AUUUCGAGCAGGCUGAAAAAAAGGUUGCAGAACUUUUGGCAGAAGGGCGUAA
UCAGGUUUCCGAAAUGGUUGCCAACGCCGAAAAACGUGCCGCCAAAAUUGUC
GAAGAAGCCAAAGAACAGGCUUCUUCCGAGGCGGCGCGCAUUGCAGCUCAGGC
AAAGGCCGAUGUGGAGCAGGAAUUGUUCCGCGCACGCGAAUCCCUGCGCGAUC
AGGUUGCCGUGUUGGCUGUCAAAGGUGCCGAAUCUAUUUUGCGCAGCGAAGU
CGAUGCUUCCAAACACGCAAAACUGCUCGAUACCCUGAAACAGGAGUUGUAA

cDNA: SEQ ID NO: 317

TTACAACTCCTGTTTCAGGGTATCGAGCAGTTTTGCGTGTTTGGAAGCATCGACT
TCGCTGCGCAAAATAGATTCGGCACCTTTGACAGCCAACACGGCAACCTGATCG
CGCAGGGATTCGCGTGCGCGGAACAATTCCTGCTCCACATCGGCCTTTGCCTGAG
CTGCAATGCGCGCCGCCTCGGAAGAAGCCTGTTCTTTGGCTTCTTCGACAATTTT
GGCGGCACGTTTTTCGGCGTTGGCAACCATTTCGGAAACCTGATTACGCCCTTCT
GCCAAAAGTTCTGCAACCTTTTTTTCAGCCTGCTCGAAATCGCTTTTACCACGCTC
GGCGGCAGCCAAGCCCTCGGCGATTTTTGCGGCACGCTCATCCAAAGCTTTTGCA
ATCGGCGGCCACACAAATTTCATGGTAAACCATACCAAACCGAAAAAGACGATG
ATTTGAGCGAATAATGTTGCATTGATATTCAC

### tRNA Control Transcripts

### 1. NGO_t45: NC_002946.2:cl827200-1827128

DNA (- strand): SEQ ID NO: 318

AGGCCAATAGCTCAATTGGTAGAGTATCGGTCTCCAAAACCGAGGGTTGGGGGT
TCGAGACCCTCTTGGCCTG

RNA: SEQ ID NO: 319

AGGCCAAUAGCUCAAUUGGUAGAGUAUCGGUCUCCAAAACCGAGGGUUGGGG
GUUCGAGACCCUCUUGGCCUG

cDNA: SEQ ID NO: 320

CAGGCCAAGAGGGTCTCGAACCCCCAACCCTCGGTTTTGGAGACCGATACTCTA
CCAATTGAGCTATTGGCCT

### 2. NGO_t47: NC_002946.2:c1828597-1828527

DNA (- strand) SEQ ID NO: 321

GCGGGTGTAGCTCAATGGTAGAGCAGAAGCCTTCCAAGCTTACGGTGAGGGTTC
GATTCCCTTCACCCGCT

RNA: SEQ ID NO: 322

GCGGGUGUAGCUCAAUGGUAGAGCAGAAGCCUUCCAAGCUUACGGUGAGGGU
UCGAUUCCCUUCACCCGCU

cDNA: SEQ ID NO: 323

AGCGGGTGAAGGGAATCGAACCCTCACCGTAAGCTTGGAAGGCTTCTGCTCTAC
CATTGAGCTACACCCGC

### Ribosomal RNA Control Transcripts

### 1. NGO_r02: NC_002946.2:c1119158-1116249

DNA (- strand): SEQ ID NO: 324

TGAAATGATAGAGTCAAGTGAATAAGTGCATCAGGCGGATGCCTTGGCGATGAT
AGGCGACGAAGGACGTGTAAGCCTGCGAAAAGCGCGGGGGAGCTGGCAATAAA
GCAATGATCCCGCGGTGTCCGAATGGGGAAACCCACTGCATTCTGTGCAGTATC
CTAAGTTGAATACATAGGCTTAGAGAAGCGAACCCGGAGAACTGAACCATCTAA
GTACCCGGAGGAAAAGAAATCAACCGAGATTCCGCAAGTAGTGGCGAGCGAAC
GCGGAGGAGCCTGTACGTAATAACTGTCGAGGTAGAAGAACAAGCTGGGAAGCT
TGACCATAGCGGGTGACAGTCCCGTATTCGAAATCTCAACAGCGGTACTAAGCG
TACGAAAAGTAGGGCGGGACACGTGAAATCCTGTCTGAATATGGGGGGACCATC
CTCCAAGGCTAAATACTCATCATCGACCGATAGTGAACCAGTACCGTGAGGGAA
AGGCGAAAAGAACCCCGGGAGGGGAGTGAAACAGAACCTGAAACCTGATGCAT
ACAAACAGTGGGAGCGCCCTAGTGGTGTGACTGCGTACCTTTTGTATAATGGGTC
AACGACTTACATTCAGTAGCGAGCTTAACCGGATAGGGGAGGCGTAGGGAAACC
GAGTCTTAATAGGGCGATGAGTTGCTGGGTGTAGACCCGAAACCGAGTGATCTA
TCCATGGCCAGGTTGAAGGTGCCGTAACAGGTACTGGAGGACCGAACCCACGCA
TGTTGCAAAATGCGGGGATGAGCTGTGGGTAGGGGTGAAAGGCTAAACAAACTC
GGAGATAGCTGGTTCTCCCCGAAAACTATTTAGGTAGTGCCTCGAGCAAGACAC
TGATGGGGGTAAAGCACTGTTATGGCTAGGGGGTTATTGCAACTTACCAACCCAT
GGCAAACTCAGAATACCATCAAGTGGTTCCTCGGGAGACAGACAGCGGGTGCTA
ACGTCCGTTGTCAAGAGGGAAACAACCCAGACCGCCGGCTAAGGTCCCAAATGA
TAGATTAAGTGGTAAACGAAGTGGGAAGGCACAGACAGCCAGGATGTTGGCTTA
GAAGCAGCCATCATTTAAAGAAAGCGTAATAGCTCACTGGTCGAGTCGTCCTGC
GCGGAAGATGTAACGGGGCTCAAATCTATAACCGAAGCTGCGGATGCCGGTTTA
CCGGCATGGTAGGGGAGCGTTCTGTAGGCTGATGAAGGTGCATTGTAAAGTGTG
CTGGAGGTATCAGAAGTGCGAATGTTGACATGAGTAGCGATAAAGCGGGTGAAA
AGCCCGCTCGCCGAAAGCCCAAGGTTTCCTACGCAACGTTCATCGGCGTAGGGT
GAGTCGGCCCCTAAGGCGAGGCAGAAATGCGTAGTCGATGGGAAACAGGTTAAT
ATTCCTGTACTTGATTCAAATGCGATGTGGGGACGGAGAAGGTTAGGTTGGCAA
GCTGTTGGAATAGCTTGTTTAAGCCGGTAGGTGGAAGACTTAGGCAAATCCGGG
TTTTCTTAACACCGAAGAAGTGATGACGAGTGTTTACGGACACGAAGCAACCGA
TACCACGCTTCCAGGAAAAGCCACTAAGCTTCAGTTTGAATCGAACCGTACCGC
AAACCGACACAGGTGGGCAGGATGAGAATTCTAAGGCGCTTGAGAGAACTCGG
GAGAAGGAACTCGGCAAATTGATACCGTAACTTCGGGAGAAGGTATGCCCTCTA
AGGTTAAGGACTTGCTCCGTAAGCCCCGGAGGGTCGCAGAGAATAGGTGGCTGC
GACTTGTTTATTAAAAACACGAGCACTCTTGCCAACACGAAAGTGGACGTATAG
GGTGTAACGCCTGCCCGGTGCCGGAAGGTTAATTGAAGATGTGCAAGCATCGGA
TCGAAGCCCCGGTAAACGGCGGCCGTAACTATAACGGTCCTAAGGTAGCGAAAT
TCCTTGTCGGGTAAGTTCCGACCCGCACGAATGGCGTAACGATGGCCACACTGTC
TCCTCCCGAGACTCAGCGAAGTTGAAGTGGTTGTGAAGATGCAATCTACCCGCTG
CTAGACGGAAAGACCCCGTGAACCTTTACTGTAGCTTTGCATTGGACTTTGAAGT
CACTTGTGTAGGATAGGTGGAAGGCTTGGAAGCAAAGACGCCAGTCTCTGTGGA
GTCGTCCTTGAAAATACCACCCTGGTGTCTTTGAGGTTCTAACCCAGACCCGTCA
TCCGGGTCGGGGACCGTGCATGGTAGGCAGTTTGACTGGGGCGGTCTCCTCCCA
AAGCGTAACGGAGGAGTTCGAAGGTTACCTAGGTCCGGTCGGAAATCGGACTGA
TAGTGCAATGGCAAAAGGTAGCTTAACTGCGAGACCGACAAGTCGGGCAGGTGC
GAAAGCAGGACATAGTGATCCGGTGGTTCTGTATGGAAGGGCCATCGCTCAACG
GATAAAAGGTACTCCGGGGATAACAGGCTTGATTCCGCCCAAGAGTTCATATCG
ACGGCGGAGTTTGGCACCTCGATGTCGGCTCATCACATCCTGGGGCTGTAGTCGG
TCCCAAGGGTATGGCTGTTCGCCATTTTAAAGTGGTACGTGAGTTGGGTTTAAAA
CGTCGTGAGACAGTTTGGTCCCTATCTGCAGTGGGCGTTGGAAGTTTGACGGGGG
CTGCTCCTAGTACGAGAGGACCGGAGTGGACGAACCTCTGGTGTACCGGTTGTA
ACGCCAGTTGCATAGCCGGGTAGCTAAGTTCGGAAGAGATAAGCGCTGAAAGCA
TCTAAGCGCGAAACTCGCCTGAAGATGAGACTTCCCTTGCGGTTTAACCGCACTA
AAGGGTCGTTCGAGACCAGGACGTTGATAGGTGGGGTGTGGAAGCGCGGTAACG
CGTGAAGCTAACCCATACTAATTGCCCGTGAGGCTTGACTCT

RNA: SEQ ID NO: 325

UGAAAUGAUAGAGUCAAGUGAAUAAGUGCAUCAGGCGGAUGCCUUGGCGAUG
AUAGGCGACGAAGGACGUGUAAGCCUGCGAAAAGCGCGGGGGAGCUGGCAAU
AAAGCAAUGAUCCCGCGGUGUCCGAAUGGGGAAACCCACUGCAUUCUGUGCA
GUAUCCUAAGUUGAAUACAUAGGCUUAGAGAAGCGAACCCGGAGAACUGAAC
CAUCUAAGUACCCGGAGGAAAAGAAAUCAACCGAGAUUCCGCAAGUAGUGGC
GAGCGAACGCGGAGGAGCCUGUACGUAAUAACUGUCGAGGUAGAAGAACAAG
CUGGGAAGCUUGACCAUAGCGGGUGACAGUCCCGUAUUCGAAAUCUCAACAG
CGGUACUAAGCGUACGAAAAGUAGGGCGGGACACGUGAAAUCCUGUCUGAAU
AUGGGGGGACCAUCCUCCAAGGCUAAAUACUCAUCAUCGACCGAUAGUGAACC
AGUACCGUGAGGGAAAGGCGAAAAGAACCCCGGGAGGGGAGUGAAACAGAAC
CUGAAACCUGAUGCAUACAAACAGUGGGAGCGCCCUAGUGGUGUGACUGCGU
ACCUUUUGUAUAAUGGGUCAACGACUUACAUUCAGUAGCGAGCUUAACCGGA
UAGGGGAGGCGUAGGGAAACCGAGUCUUAAUAGGGCGAUGAGUUGCUGGGUG
UAGACCCGAAACCGAGUGAUCUAUCCAUGGCCAGGUUGAAGGUGCCGUAACA
GGUACUGGAGGACCGAACCCACGCAUGUUGCAAAAUGCGGGGAUGAGCUGUG
GGUAGGGGUGAAAGGCUAAACAAACUCGGAGAUAGCUGGUUCUCCCCGAAAA
CUAUUUAGGUAGUGCCUCGAGCAAGACACUGAUGGGGGUAAAGCACUGUUAU
GGCUAGGGGGUUAUUGCAACUUACCAACCCAUGGCAAACUCAGAAUACCAUC
AAGUGGUUCCUCGGGAGACAGACAGCGGGUGCUAACGUCCGUUGUCAAGAGG
GAAACAACCCAGACCGCCGGCUAAGGUCCCAAAUGAUAGAUUAAGUGGUAAA
CGAAGUGGGAAGGCACAGACAGCCAGGAUGUUGGCUUAGAAGCAGCCAUCAU
UUAAAGAAAGCGUAAUAGCUCACUGGUCGAGUCGUCCUGCGCGGAAGAUGUA
ACGGGGCUCAAAUCUAUAACCGAAGCUGCGGAUGCCGGUUUACCGGCAUGGU
AGGGGAGCGUUCUGUAGGCUGAUGAAGGUGCAUUGUAAAGUGUGCUGGAGGU
AUCAGAAGUGCGAAUGUUGACAUGAGUAGCGAUAAAGCGGGUGAAAAGCCCG
CUCGCCGAAAGCCCAAGGUUUCCUACGCAACGUUCAUCGGCGUAGGGUGAGUC
GGCCCCUAAGGCGAGGCAGAAAUGCGUAGUCGAUGGGAAACAGGUUAAUAUU
CCUGUACUUGAUUCAAAUGCGAUGUGGGGACGGAGAAGGUUAGGUUGGCAAG
CUGUUGGAAUAGCUUGUUUAAGCCGGUAGGUGGAAGACUUAGGCAAAUCCGG
GUUUUCUUAACACCGAAGAAGUGAUGACGAGUGUUUACGGACACGAAGCAAC
CGAUACCACGCUUCCAGGAAAAGCCACUAAGCUUCAGUUUGAAUCGAACCGUA
CCGCAAACCGACACAGGUGGGCAGGAUGAGAAUUCUAAGGCGCUUGAGAGAA
CUCGGGAGAAGGAACUCGGCAAAUUGAUACCGUAACUUCGGGAGAAGGUAUG
CCCUCUAAGGUUAAGGACUUGCUCCGUAAGCCCCGGAGGGUCGCAGAGAAUA
GGUGGCUGCGACUUGUUUAUUAAAAACACGAGCACUCUUGCCAACACGAAAG
UGGACGUAUAGGGUGUAACGCCUGCCCGGUGCCGGAAGGUUAAUUGAAGAUG
UGCAAGCAUCGGAUCGAAGCCCCGGUAAACGGCGGCCGUAACUAUAACGGUCC
UAAGGUAGCGAAAUUCCUUGUCGGGUAAGUUCCGACCCGCACGAAUGGCGUA
ACGAUGGCCACACUGUCUCCUCCCGAGACUCAGCGAAGUUGAAGUGGUUGUG
AAGAUGCAAUCUACCCGCUGCUAGACGGAAAGACCCCGUGAACCUUUACUGUA
GCUUUGCAUUGGACUUUGAAGUCACUUGUGUAGGAUAGGUGGAAGGCUUGGA
AGCAAAGACGCCAGUCUCUGUGGAGUCGUCCUUGAAAAUACCACCCUGGUGUC
UUUGAGGUUCUAACCCAGACCCGUCAUCCGGGUCGGGGACCGUGCAUGGUAG
GCAGUUUGACUGGGGCGGUCUCCUCCCAAAGCGUAACGGAGGAGUUCGAAGG
UUACCUAGGUCCGGUCGGAAAUCGGACUGAUAGUGCAAUGGCAAAAGGUAGC
UUAACUGCGAGACCGACAAGUCGGGCAGGUGCGAAAGCAGGACAUAGUGAUC
CGGUGGUUCUGUAUGGAAGGGCCAUCGCUCAACGGAUAAAAGGUACUCCGGG
GAUAACAGGCUUGAUUCCGCCCAAGAGUUCAUAUCGACGGCGGAGUUUGGCA
CCUCGAUGUCGGCUCAUCACAUCCUGGGGCUGUAGUCGGUCCCAAGGGUAUGG
CUGUUCGCCAUUUUAAAGUGGUACGUGAGUUGGGUUUAAAACGUCGUGAGAC
AGUUUGGUCCCUAUCUGCAGUGGGCGUUGGAAGUUUGACGGGGGCUGCUCCU
AGUACGAGAGGACCGGAGUGGACGAACCUCUGGUGUACCGGUUGUAACGCCA
GUUGCAUAGCCGGGUAGCUAAGUUCGGAAGAGAUAAGCGCUGAAAGCAUCUA
AGCGCGAAACUCGCCUGAAGAUGAGACUUCCCUUGCGGUUUAACCGCACUAAA
GGGUCGUUCGAGACCAGGACGUUGAUAGGUGGGGUGUGGAAGCGCGGUAACG
CGUGAAGCUAACCCAUACUAAUUGCCCGUGAGGCUUGACUCU

cDNA: SEQ ID NO: 326

AGAGTCAAGCCTCACGGGCAATTAGTATGGGTTAGCTTCACGCGTTACCGCGCTT
CCACACCCCACCTATCAACGTCCTGGTCTCGAACGACCCTTTAGTGCGGTTAAAC
CGCAAGGGAAGTCTCATCTTCAGGCGAGTTTCGCGCTTAGATGCTTTCAGCGCTT
ATCTCTTCCGAACTTAGCTACCCGGCTATGCAACTGGCGTTACAACCGGTACACC
AGAGGTTCGTCCACTCCGGTCCTCTCGTACTAGGAGCAGCCCCCGTCAAACTTCC
AACGCCCACTGCAGATAGGGACCAAACTGTCTCACGACGTTTTAAACCCAACTC
ACGTACCACTTTAAAATGGCGAACAGCCATACCCTTGGGACCGACTACAGCCCC
AGGATGTGATGAGCCGACATCGAGGTGCCAAACTCCGCCGTCGATATGAACTCT
TGGGCGGAATCAAGCCTGTTATCCCCGGAGTACCTTTTATCCGTTGAGCGATGGC
CCTTCCATACAGAACCACCGGATCACTATGTCCTGCTTTCGCACCTGCCCGACTT
GTCGGTCTCGCAGTTAAGCTACCTTTTGCCATTGCACTATCAGTCCGATTTCCGAC
CGGACCTAGGTAACCTTCGAACTCCTCCGTTACGCTTTGGGAGGAGACCGCCCCA
GTCAAACTGCCTACCATGCACGGTCCCCGACCCGGATGACGGGTCTGGGTTAGA
ACCTCAAAGACACCAGGGTGGTATTTTCAAGGACGACTCCACAGAGACTGGCGT
CTTTGCTTCCAAGCCTTCCACCTATCCTACACAAGTGACTTCAAAGTCCAATGCA
AAGCTACAGTAAAGGTTCACGGGGTCTTTCCGTCTAGCAGCGGGTAGATTGCATC
TTCACAACCACTTCAACTTCGCTGAGTCTCGGGAGGAGACAGTGTGGCCATCGTT
ACGCCATTCGTGCGGGTCGGAACTTACCCGACAAGGAATTTCGCTACCTTAGGA
CCGTTATAGTTACGGCCGCCGTTTACCGGGGCTTCGATCCGATGCTTGCACATCT
TCAATTAACCTTCCGGCACCGGGCAGGCGTTACACCCTATACGTCCACTTTCGTG
TTGGCAAGAGTGCTCGTGTTTTTAATAAACAAGTCGCAGCCACCTATTCTCTGCG
ACCCTCCGGGGCTTACGGAGCAAGTCCTTAACCTTAGAGGGCATACCTTCTCCCG
AAGTTACGGTATCAATTTGCCGAGTTCCTTCTCCCGAGTTCTCTCAAGCGCCTTA
GAATTCTCATCCTGCCCACCTGTGTCGGTTTGCGGTACGGTTCGATTCAAACTGA
AGCTTAGTGGCTTTTCCTGGAAGCGTGGTATCGGTTGCTTCGTGTCCGTAAACAC
TCGTCATCACTTCTTCGGTGTTAAGAAAACCCGGATTTGCCTAAGTCTTCCACCT
ACCGGCTTAAACAAGCTATTCCAACAGCTTGCCAACCTAACCTTCTCCGTCCCCA
CATCGCATTTGAATCAAGTACAGGAATATTAACCTGTTTCCCATCGACTACGCAT
TTCTGCCTCGCCTTAGGGGCCGACTCACCCTACGCCGATGAACGTTGCGTAGGAA
ACCTTGGGCTTTCGGCGAGCGGGCTTTTCACCCGCTTTATCGCTACTCATGTCAA
CATTCGCACTTCTGATACCTCCAGCACACTTTACAATGCACCTTCATCAGCCTAC
AGAACGCTCCCCTACCATGCCGGTAAACCGGCATCCGCAGCTTCGGTTATAGATT
TGAGCCCCGTTACATCTTCCGCGCAGGACGACTCGACCAGTGAGCTATTACGCTT
TCTTTAAATGATGGCTGCTTCTAAGCCAACATCCTGGCTGTCTGTGCCTTCCCACT
TCGTTTACCACTTAATCTATCATTTGGGACCTTAGCCGGCGGTCTGGGTTGTTTCC
CTCTTGACAACGGACGTTAGCACCCGCTGTCTGTCTCCCGAGGAACCACTTGATG
GTATTCTGAGTTTGCCATGGGTTGGTAAGTTGCAATAACCCCCTAGCCATAACAG
TGCTTTACCCCCATCAGTGTCTTGCTCGAGGCACTACCTAAATAGTTTTCGGGGA
GAACCAGCTATCTCCGAGTTTGTTTAGCCTTTCACCCCTACCCACAGCTCATCCC
CGCATTTTGCAACATGCGTGGGTTCGGTCCTCCAGTACCTGTTACGGCACCTTCA
ACCTGGCCATGGATAGATCACTCGGTTTCGGGTCTACACCCAGCAACTCATCGCC
CTATTAAGACTCGGTTTCCCTACGCCTCCCCTATCCGGTTAAGCTCGCTACTGAA
TGTAAGTCGTTGACCCATTATACAAAAGGTACGCAGTCACACCACTAGGGCGCT
CCCACTGTTTGTATGCATCAGGTTTCAGGTTCTGTTTCACTCCCCTCCCGGGGTTC
TTTTCGCCTTTCCCTCACGGTACTGGTTCACTATCGGTCGATGATGAGTATTTAGC
CTTGGAGGATGGTCCCCCCATATTCAGACAGGATTTCACGTGTCCCGCCCTACTT
TTCGTACGCTTAGTACCGCTGTTGAGATTTCGAATACGGGACTGTCACCCGCTAT
GGTCAAGCTTCCCAGCTTGTTCTTCTACCTCGACAGTTATTACGTACAGGCTCCTC
CGCGTTCGCTCGCCACTACTTGCGGAATCTCGGTTGATTTCTTTTCCTCCGGGTAC
TTAGATGGTTCAGTTCTCCGGGTTCGCTTCTCTAAGCCTATGTATTCAACTTAGGA
TACTGCACAGAATGCAGTGGGTTTCCCCATTCGGACACCGCGGGATCATTGCTTT
ATTGCCAGCTCCCCCGCGCTTTTCGCAGGCTTACACGTCCTTCGTCGCCTATCATC
GCCAAGGCATCCGCCTGATGCACTTATTCACTTGACTCTATCATTTCA

### 2. NGO_r03: NC_002946.2:c1121298-1119754

DNA (- strand): SEQ ID NO: 327

TGAACATAAGAGTTTGATCCTGGCTCAGATTGAACGCTGGCGGCATGCTTTACAC
ATGCAAGTCGGACGGCAGCACAGGGAAGCTTGCTTCTCGGGTGGCGAGTGGCGA
ACGGGTGAGTAACATATCGGAACGTACCGGGTAGCGGGGGATAACTGATCGAAA
GATCAGCTAATACCGCATACGTCTTGAGAGGGAAAGCAGGGGACCTTCGGGCCT
TGCGCTATCCGAGCGGCCGATATCTGATTAGCTGGTTGGCGGGGTAAAGGCCCA
CCAAGGCGACGATCAGTAGCGGGTCTGAGAGGATGATCCGCCACACTGGGACTG
AGACACGGCCCAGACTCCTACGGGAGGCAGCAGTGGGGAATTTTGGACAATGGG
CGCAAGCCTGATCCAGCCATGCCGCGTGTCTGAAGAAGGCCTTCGGGTTGTAAA
GGACTTTTGTCAGGGAAGAAAAGGCCGTTGCCAATATCGGCGGCCGATGACGGT
ACCTGAAGAATAAGCACCGGCTAACTACGTGCCAGCAGCCGCGGTAATACGTAG
GGTGCGAGCGTTAATCGGAATTACTGGGCGTAAAGCGGGCGCAGACGGTTACTT
AAGCAGGATGTGAAATCCCCGGGCTCAACCCGGGAACTGCGTTCTGAACTGGGT
GACTCGAGTGTGTCAGAGGGAGGTGGAATTCCACGTGTAGCAGTGAAATGCGTA
GAGATGTGGAGGAATACCGATGGCGAAGGCAGCCTCCTGGGATAACACTGACGT
TCATGTCCGAAAGCGTGGGTAGCAAACAGGATTAGATACCCTGGTAGTCCACGC
CCTAAACGATGTCAATTAGCTGTTGGGCAACTTGATTGCTTGGTAGCGTAGCTAA
CGCGTGAAATTGACCGCCTGGGGAGTACGGTCGCAAGATTAAAACTCAAAGGAA
TTGACGGGGACCCGCACAAGCGGTGGATGATGTGGATTAATTCGATGCAACGCG
AAGAACCTTACCTGGTTTTGACATGTGCGGAATCCTCCGGAGACGGAGGAGTGC
CTTCGGGAGCCGTAACACAGGTGCTGCATGGCTGTCGTCAGCTCGTGTCGTGAGA
TGTTGGGTTAAGTCCCGCAACGAGCGCAACCCTTGTCATTAGTTGCCATCATTCG
GTTGGGCACTCTAATGAGACTGCCGGTGACAAGCCGGAGGAAGGTGGGGATGAC
GTCAAGTCCTCATGGCCCTTATGACCAGGGCTTCACACGTCATACAATGGTCGGT
ACAGAGGGTAGCCAAGCCGCGAGGCGGAGCCAATCTCACAAAACCGATCGTAG
TCCGGATTGCACTCTGCAACTCGAGTGCATGAAGTCGGAATCGCTAGTAATCGCA
GGTCAGCATACTGCGGTGAATACGTTCCCGGGTCTTGTACACACCGCCCGTCACA
CCATGGGAGTGGGGGATACCAGAAGTAGGTAGGGTAACCGCAAGGAGTCCGCTT
ACCACGGTATGCTTCATGACTGGGGTGAAGTCGTAACAAGGTAGCCGTAGGGGA
ACCTGCGGCTGGATCACCTCCTTTCTA

RNA: SEQ ID NO: 328

UGAACAUAAGAGUUUGAUCCUGGCUCAGAUUGAACGCUGGCGGCAUGCUUUA
CACAUGCAAGUCGGACGGCAGCACAGGGAAGCUUGCUUCUCGGGUGGCGAGU
GGCGAACGGGUGAGUAACAUAUCGGAACGUACCGGGUAGCGGGGGAUAACUG
AUCGAAAGAUCAGCUAAUACCGCAUACGUCUUGAGAGGGAAAGCAGGGGACC
UUCGGGCCUUGCGCUAUCCGAGCGGCCGAUAUCUGAUUAGCUGGUUGGCGGG
GUAAAGGCCCACCAAGGCGACGAUCAGUAGCGGGUCUGAGAGGAUGAUCCGC
CACACUGGGACUGAGACACGGCCCAGACUCCUACGGGAGGCAGCAGUGGGGAA
UUUUGGACAAUGGGCGCAAGCCUGAUCCAGCCAUGCCGCGUGUCUGAAGAAG
GCCUUCGGGUUGUAAAGGACUUUUGUCAGGGAAGAAAAGGCCGUUGCCAAUA
UCGGCGGCCGAUGACGGUACCUGAAGAAUAAGCACCGGCUAACUACGUGCCAG
CAGCCGCGGUAAUACGUAGGGUGCGAGCGUUAAUCGGAAUUACUGGGCGUAA
AGCGGGCGCAGACGGUUACUUAAGCAGGAUGUGAAAUCCCCGGGCUCAACCCG
GGAACUGCGUUCUGAACUGGGUGACUCGAGUGUGUCAGAGGGAGGUGGAAUU
CCACGUGUAGCAGUGAAAUGCGUAGAGAUGUGGAGGAAUACCGAUGGCGAAG
GCAGCCUCCUGGGAUAACACUGACGUUCAUGUCCGAAAGCGUGGGUAGCAAA
CAGGAUUAGAUACCCUGGUAGUCCACGCCCUAAACGAUGUCAAUUAGCUGUU
GGGCAACUUGAUUGCUUGGUAGCGUAGCUAACGCGUGAAAUUGACCGCCUGG
GGAGUACGGUCGCAAGAUUAAAACUCAAAGGAAUUGACGGGGACCCGCACAA
GCGGUGGAUGAUGUGGAUUAAUUCGAUGCAACGCGAAGAACCUUACCUGGUU
UUGACAUGUGCGGAAUCCUCCGGAGACGGAGGAGUGCCUUCGGGAGCCGUAA
CACAGGUGCUGCAUGGCUGUCGUCAGCUCGUGUCGUGAGAUGUUGGGUUAAG
UCCCGCAACGAGCGCAACCCUUGUCAUUAGUUGCCAUCAUUCGGUUGGGCACU
CUAAUGAGACUGCCGGUGACAAGCCGGAGGAAGGUGGGGAUGACGUCAAGUC
CUCAUGGCCCUUAUGACCAGGGCUUCACACGUCAUACAAUGGUCGGUACAGAG
GGUAGCCAAGCCGCGAGGCGGAGCCAAUCUCACAAAACCGAUCGUAGUCCGGA
UUGCACUCUGCAACUCGAGUGCAUGAAGUCGGAAUCGCUAGUAAUCGCAGGU
CAGCAUACUGCGGUGAAUACGUUCCCGGGUCUUGUACACACCGCCCGUCACAC
CAUGGGAGUGGGGGAUACCAGAAGUAGGUAGGGUAACCGCAAGGAGUCCGCU
UACCACGGUAUGCUUCAUGACUGGGGUGAAGUCGUAACAAGGUAGCCGUAGG
GGAACCUGCGGCUGGAUCACCUCCUUUCUA

cDNA: SEQ ID NO: 329

TAGAAAGGAGGTGATCCAGCCGCAGGTTCCCCTACGGCTACCTTGTTACGACTTC
ACCCCAGTCATGAAGCATACCGTGGTAAGCGGACTCCTTGCGGTTACCCTACCTA
CTTCTGGTATCCCCCACTCCCATGGTGTGACGGGCGGTGTGTACAAGACCCGGGA
ACGTATTCACCGCAGTATGCTGACCTGCGATTACTAGCGATTCCGACTTCATGCA
CTCGAGTTGCAGAGTGCAATCCGGACTACGATCGGTTTTGTGAGATTGGCTCCGC
CTCGCGGCTTGGCTACCCTCTGTACCGACCATTGTATGACGTGTGAAGCCCTGGT
CATAAGGGCCATGAGGACTTGACGTCATCCCCACCTTCCTCCGGCTTGTCACCGG
CAGTCTCATTAGAGTGCCCAACCGAATGATGGCAACTAATGACAAGGGTTGCGC
TCGTTGCGGGACTTAACCCAACATCTCACGACACGAGCTGACGACAGCCATGCA
GCACCTGTGTTACGGCTCCCGAAGGCACTCCTCCGTCTCCGGAGGATTCCGCACA
TGTCAAAACCAGGTAAGGTTCTTCGCGTTGCATCGAATTAATCCACATCATCCAC
CGCTTGTGCGGGTCCCCGTCAATTCCTTTGAGTTTTAATCTTGCGACCGTACTCCC
CAGGCGGTCAATTTCACGCGTTAGCTACGCTACCAAGCAATCAAGTTGCCCAAC
AGCTAATTGACATCGTTTAGGGCGTGGACTACCAGGGTATCTAATCCTGTTTGCT
ACCCACGCTTTCGGACATGAACGTCAGTGTTATCCCAGGAGGCTGCCTTCGCCAT
CGGTATTCCTCCACATCTCTACGCATTTCACTGCTACACGTGGAATTCCACCTCCC
TCTGACACACTCGAGTCACCCAGTTCAGAACGCAGTTCCCGGGTTGAGCCCGGG
GATTTCACATCCTGCTTAAGTAACCGTCTGCGCCCGCTTTACGCCCAGTAATTCC
GATTAACGCTCGCACCCTACGTATTACCGCGGCTGCTGGCACGTAGTTAGCCGGT
GCTTATTCTTCAGGTACCGTCATCGGCCGCCGATATTGGCAACGGCCTTTTCTTCC
CTGACAAAAGTCCTTTACAACCCGAAGGCCTTCTTCAGACACGCGGCATGGCTG
GATCAGGCTTGCGCCCATTGTCCAAAATTCCCCACTGCTGCCTCCCGTAGGAGTC
TGGGCCGTGTCTCAGTCCCAGTGTGGCGGATCATCCTCTCAGACCCGCTACTGAT
CGTCGCCTTGGTGGGCCTTTACCCCGCCAACCAGCTAATCAGATATCGGCCGCTC
GGATAGCGCAAGGCCCGAAGGTCCCCTGCTTTCCCTCTCAAGACGTATGCGGTAT
TAGCTGATCTTTCGATCAGTTATCCCCCGCTACCCGGTACGTTCCGATATGTTACT
CACCCGTTCGCCACTCGCCACCCGAGAAGCAAGCTTCCCTGTGCTGCCGTCCGAC
TTGCATGTGTAAAGCATGCCGCCAGCGTTCAATCTGAGCCAGGATCAAACTCTTA
TGTTCA

### 3. NGO_r05: NC_002946.2:c1261255-1258352

DNA (- strand): SEQ ID NO: 330

TGAAATGATAGAGTCAAGTGAATAAGTGCATCAGGCGGATGCCTTGGCGATGAT
AGGCGACGAAGGACGTGTAAGCCTGCGAAAAGCGCGGGGGAGCTGGCAATAAA
GCAATGATCCCGCGGTGTCCGAATGGGGAAACCCACTGCATTCTGTGCAGTATC
CTAAGTTGAATACATAGGCTTAGAGAAGCGAACCCGGAGAACTGAACCATCTAA
GTACCCGGAGGAAAAGAAATCAACCGAGATTCCGCAAGTAGTGGCGAGCGAAC
GCGGAGGAGCCTGTACGTAATAACTGTCGAGGTAGAAGAACAAGCTGGGAAGCT
TGACCATAGCGGGTGACAGTCCCGTATTCGAAATCTCAACAGCGGTACTAAGCG
TACGAAAAGTAGGGCGGGACACGTGAAATCCTGTCTGAATATGGGGGGACCATC
CTCCAAGGCTAAATACTCATCATCGACCGATAGTGAACCAGTACCGTGAGGGAA
AGGCGAAAAGAACCCCGGGAGGGGAGTGAAACAGAACCTGAAACCTGATGCAT
ACAAACAGTGGGAGCGCCCTAGTGGTGTGACTGCGTACCTTTTGTATAATGGGTC
AACGACTTACATTCAGTAGCGAGCTTAACCGGATAGGGGAGGCGTAGGGAAACC
GAGTCTTAATAGGGCGATGAGTTGCTGGGTGTAGACCCGAAACCGAGTGATCTA
TCCATGGCCAGGTTGAAGGTGCCGTAACAGGTACTGGAGGACCGAACCCACGCA
TGTTGCAAAATGCGGGGATGAGCTGTGGGTAGGGGTGAAAGGCTAAACAAACTC
GGAGATAGCTGGTTCTCCCCGAAAACTATTTAGGTAGTGCCTCGAGCAAGACAC
TGATGGGGGTAAAGCACTGTTATGGCTAGGGGGTTATTGCAACTTACCAACCCAT
GGCAAACTCAGAATACCATCAAGTGGTTCCTCGGGAGACAGACAGCGGGTGCTA
ACGTCCGTTGTCAAGAGGGAAACAACCCAGACCGCCGGCTAAGGTCCCAAATGA
TAGATTAAGTGGTAAACGAAGTGGGAAGGCACAGACAGCCAGGATGTTGGCTTA
GAAGCAGCCATCATTTAAAGAAAGCGTAATAGCTCACTGGTCGAGTCGTCCTGC
GCGGAAGATGTAACGGGGCTCAAATCTATAACCCAAGCTGCGTATGCCGGTTTA
CCGGCATGGTAGGGGAGCGTTCTGTAGGCTGATGAAGGTGCATTGTAAAGTGTG
CTGGAGGTATCAGAAGTGCGAATGTTGACATGAGTAGCGATAAAGCGGGTGAAA
AGCCCGCTCGCCGCAAAGCCCAAGGTTTCCTACGCAACGTTCATCGGCGTAGGG
TGAGTCGGCCCCTAAGGCGAGGCAGAAATGCGTAGTCGATGGGAAACAGGTTAA
TATTCCTGTACTTGATTCAAATGCGATGTGGGGACGGAGAAGGTTAGGTTGGCA
AGCTGTTGGAATAGCTTGTTTAAGCCGGTAGGTGGAAGACTTAGGCAAATCCGG
GTTTTCTTAACACCGAGAAGTGATGACGAGTGTCTACGGACACGAAGCAACCGA
TACCACGCTTCCAGGAAAAGCCACTAAGCTTCAGTTTGAATCGAACCGTACCGC
AAACCGACACAGGTGGGCAGGATGAGAATTCTAAGGCGCTTGAGAGAACTCGG
GAGAAGGAACTCGGCAAATTGATACCGTAACTTCGGGAGAAGGTATGCCCTCTA
AGGTTAAGGACTTGCTCCGTAAGCCCCGGAGGGTCGCAGAGAATAGGTGGCTGC
GACTGTTTATTAAAAACACAGCACTCTGCCAACACGAAAGTGGACGTATAGGGT
GTGACGCCTGCCCGGTGCCGGAAGGTTAATTGAAGATGTGCAAGCATCGGATCG
AAGCCCCGGTAAACGGCGGCCGTAACTATAACGGTCCTAAGGTAGCGAAATTCC
TTGTCGGGTAAGTTCCGACCCGCACGAATGGCGTAACGATGGCCACACTGTCTCC
TCCCGAGACTCAGCGAAGTTGAAGTGGTTGTGAAGATGCAATCTACCCGCTGCT
AGACGGAAAGACCCCGTGAACCTTTACTGTAGCTTTGCATTGGACTTTGAAGTCA
CTTGTGTAGGATAGGTGGGAGGCTTGGAAGCAGAGACGCCAGTCTCTGTGGAGT
CGTCCTTGAAATACCACCCTGGTGTCTTTGAGGTTCTAACCCAGACCCGTCATCC
GGGTCGGGGACCGTGCATGGTAGGCAGTTTGACTGGGGCGGTCTCCTCCCAAAG
CGTAACGGAGGAGTTCGAAGGTTACCTAGGTCCGGTCGGAAATCGGACTGATAG
TGCAATGGCAAAAGGTAGCTTAACTGCGAGACCGACAAGTCGGGCAGGTGCGAA
AGCAGGACATAGTGATCCGGTGGTTCTGTATGGAAGGGCCATCGCTCAACGGAT
AAAAGGTACTCCGGGGATAACAGGCTGATTCCGCCCAAGAGTTCATATCGACGG
CGGAGTTTGGCACCTCGATGTCGGCTCATCACATCCTGGGGCTGTAGTCGGTCCC
AAGGGTATGGCTGTTCGCCATTTAAAGTGGTACGTGAGCTGGGTTTAAAACGTCG
TGAGACAGTTTGGTCCCTATCTGCAGTGGGCGTTGGAAGTTTGACGGGGGCTGCT
CCTAGTACGAGAGGACCGGAGTGGACGAACCTCTGGTGTACCGGTTGTAACGCC
AGTTGCATAGCCGGGTAGCTAAGTTCGGAAGAGATAAGCGCTGAAAGCATCTAA
GCGCGAAACTCGCCTGAAGATGAGACTTCCCTTGCGGTTTAACCGCACTAAAGG
GTCGTTCGAGACCAGGACGTTGATAGGTGGGGTGTGGAAGCGCGGTAACGCGTG
AAGCTAACCCATACTAATTGCCCGTGAGGCTTGACTCT

RNA: SEQ ID NO: 331

UGAAAUGAUAGAGUCAAGUGAAUAAGUGCAUCAGGCGGAUGCCUUGGCGAUG
AUAGGCGACGAAGGACGUGUAAGCCUGCGAAAAGCGCGGGGGAGCUGGCAAU
AAAGCAAUGAUCCCGCGGUGUCCGAAUGGGGAAACCCACUGCAUUCUGUGCA
GUAUCCUAAGUUGAAUACAUAGGCUUAGAGAAGCGAACCCGGAGAACUGAAC
CAUCUAAGUACCCGGAGGAAAAGAAAUCAACCGAGAUUCCGCAAGUAGUGGC
GAGCGAACGCGGAGGAGCCUGUACGUAAUAACUGUCGAGGUAGAAGAACAAG
CUGGGAAGCUUGACCAUAGCGGGUGACAGUCCCGUAUUCGAAAUCUCAACAG
CGGUACUAAGCGUACGAAAAGUAGGGCGGGACACGUGAAAUCCUGUCUGAAU
AUGGGGGGACCAUCCUCCAAGGCUAAAUACUCAUCAUCGACCGAUAGUGAACC
AGUACCGUGAGGGAAAGGCGAAAAGAACCCCGGGAGGGGAGUGAAACAGAAC
CUGAAACCUGAUGCAUACAAACAGUGGGAGCGCCCUAGUGGUGUGACUGCGU
ACCUUUUGUAUAAUGGGUCAACGACUUACAUUCAGUAGCGAGCUUAACCGGA
UAGGGGAGGCGUAGGGAAACCGAGUCUUAAUAGGGCGAUGAGUUGCUGGGUG
UAGACCCGAAACCGAGUGAUCUAUCCAUGGCCAGGUUGAAGGUGCCGUAACA
GGUACUGGAGGACCGAACCCACGCAUGUUGCAAAAUGCGGGGAUGAGCUGUG
GGUAGGGGUGAAAGGCUAAACAAACUCGGAGAUAGCUGGUUCUCCCCGAAAA
CUAUUUAGGUAGUGCCUCGAGCAAGACACUGAUGGGGGUAAAGCACUGUUAU
GGCUAGGGGGUUAUUGCAACUUACCAACCCAUGGCAAACUCAGAAUACCAUC
AAGUGGUUCCUCGGGAGACAGACAGCGGGUGCUAACGUCCGUUGUCAAGAGG
GAAACAACCCAGACCGCCGGCUAAGGUCCCAAAUGAUAGAUUAAGUGGUAAA
CGAAGUGGGAAGGCACAGACAGCCAGGAUGUUGGCUUAGAAGCAGCCAUCAU
UUAAAGAAAGCGUAAUAGCUCACUGGUCGAGUCGUCCUGCGCGGAAGAUGUA
ACGGGGCUCAAAUCUAUAACCCAAGCUGCGUAUGCCGGUUUACCGGCAUGGU
AGGGGAGCGUUCUGUAGGCUGAUGAAGGUGCAUUGUAAAGUGUGCUGGAGGU
AUCAGAAGUGCGAAUGUUGACAUGAGUAGCGAUAAAGCGGGUGAAAAGCCCG
CUCGCCGCAAAGCCCAAGGUUUCCUACGCAACGUUCAUCGGCGUAGGGUGAGU
CGGCCCCUAAGGCGAGGCAGAAAUGCGUAGUCGAUGGGAAACAGGUUAAUAU
UCCUGUACUUGAUUCAAAUGCGAUGUGGGGACGGAGAAGGUUAGGUUGGCAA
GCUGUUGGAAUAGCUUGUUUAAGCCGGUAGGUGGAAGACUUAGGCAAAUCCG
GGUUUUCUUAACACCGAGAAGUGAUGACGAGUGUCUACGGACACGAAGCAAC
CGAUACCACGCUUCCAGGAAAAGCCACUAAGCUUCAGUUUGAAUCGAACCGUA
CCGCAAACCGACACAGGUGGGCAGGAUGAGAAUUCUAAGGCGCUUGAGAGAA
CUCGGGAGAAGGAACUCGGCAAAUUGAUACCGUAACUUCGGGAGAAGGUAUG
CCCUCUAAGGUUAAGGACUUGCUCCGUAAGCCCCGGAGGGUCGCAGAGAAUA
GGUGGCUGCGACUGUUUAUUAAAAACACAGCACUCUGCCAACACGAAAGUGG
ACGUAUAGGGUGUGACGCCUGCCCGGUGCCGGAAGGUUAAUUGAAGAUGUGC
AAGCAUCGGAUCGAAGCCCCGGUAAACGGCGGCCGUAACUAUAACGGUCCUAA
GGUAGCGAAAUUCCUUGUCGGGUAAGUUCCGACCCGCACGAAUGGCGUAACG
AUGGCCACACUGUCUCCUCCCGAGACUCAGCGAAGUUGAAGUGGUUGUGAAG
AUGCAAUCUACCCGCUGCUAGACGGAAAGACCCCGUGAACCUUUACUGUAGCU
UUGCAUUGGACUUUGAAGUCACUUGUGUAGGAUAGGUGGGAGGCUUGGAAGC
AGAGACGCCAGUCUCUGUGGAGUCGUCCUUGAAAUACCACCCUGGUGUCUUU
GAGGUUCUAACCCAGACCCGUCAUCCGGGUCGGGGACCGUGCAUGGUAGGCAG
UUUGACUGGGGCGGUCUCCUCCCAAAGCGUAACGGAGGAGUUCGAAGGUUAC
CUAGGUCCGGUCGGAAAUCGGACUGAUAGUGCAAUGGCAAAAGGUAGCUUAA
CUGCGAGACCGACAAGUCGGGCAGGUGCGAAAGCAGGACAUAGUGAUCCGGU
GGUUCUGUAUGGAAGGGCCAUCGCUCAACGGAUAAAAGGUACUCCGGGGAUA
ACAGGCUGAUUCCGCCCAAGAGUUCAUAUCGACGGCGGAGUUUGGCACCUCGA
UGUCGGCUCAUCACAUCCUGGGGCUGUAGUCGGUCCCAAGGGUAUGGCUGUU
CGCCAUUUAAAGUGGUACGUGAGCUGGGUUUAAAACGUCGUGAGACAGUUUG
GUCCCUAUCUGCAGUGGGCGUUGGAAGUUUGACGGGGGCUGCUCCUAGUACG
AGAGGACCGGAGUGGACGAACCUCUGGUGUACCGGUUGUAACGCCAGUUGCA
UAGCCGGGUAGCUAAGUUCGGAAGAGAUAAGCGCUGAAAGCAUCUAAGCGCG
AAACUCGCCUGAAGAUGAGACUUCCCUUGCGGUUUAACCGCACUAAAGGGUC
GUUCGAGACCAGGACGUUGAUAGGUGGGGUGUGGAAGCGCGGUAACGCGUGA
AGCUAACCCAUACUAAUUGCCCGUGAGGCUUGACUCU

cDNA: SEQ ID NO: 332

AGAGTCAAGCCTCACGGGCAATTAGTATGGGTTAGCTTCACGCGTTACCGCGCTT
CCACACCCCACCTATCAACGTCCTGGTCTCGAACGACCCTTTAGTGCGGTTAAAC
CGCAAGGGAAGTCTCATCTTCAGGCGAGTTTCGCGCTTAGATGCTTTCAGCGCTT
ATCTCTTCCGAACTTAGCTACCCGGCTATGCAACTGGCGTTACAACCGGTACACC
AGAGGTTCGTCCACTCCGGTCCTCTCGTACTAGGAGCAGCCCCCGTCAAACTTCC
AACGCCCACTGCAGATAGGGACCAAACTGTCTCACGACGTTTTAAACCCAGCTC
ACGTACCACTTTAAATGGCGAACAGCCATACCCTTGGGACCGACTACAGCCCCA
GGATGTGATGAGCCGACATCGAGGTGCCAAACTCCGCCGTCGATATGAACTCTT
GGGCGGAATCAGCCTGTTATCCCCGGAGTACCTTTTATCCGTTGAGCGATGGCCC
TTCCATACAGAACCACCGGATCACTATGTCCTGCTTTCGCACCTGCCCGACTTGT
CGGTCTCGCAGTTAAGCTACCTTTTGCCATTGCACTATCAGTCCGATTTCCGACC
GGACCTAGGTAACCTTCGAACTCCTCCGTTACGCTTTGGGAGGAGACCGCCCCA
GTCAAACTGCCTACCATGCACGGTCCCCGACCCGGATGACGGGTCTGGGTTAGA
ACCTCAAAGACACCAGGGTGGTATTTCAAGGACGACTCCACAGAGACTGGCGTC
TCTGCTTCCAAGCCTCCCACCTATCCTACACAAGTGACTTCAAAGTCCAATGCAA
AGCTACAGTAAAGGTTCACGGGGTCTTTCCGTCTAGCAGCGGGTAGATTGCATCT
TCACAACCACTTCAACTTCGCTGAGTCTCGGGAGGAGACAGTGTGGCCATCGTTA
CGCCATTCGTGCGGGTCGGAACTTACCCGACAAGGAATTTCGCTACCTTAGGACC
GTTATAGTTACGGCCGCCGTTTACCGGGGCTTCGATCCGATGCTTGCACATCTTC
AATTAACCTTCCGGCACCGGGCAGGCGTCACACCCTATACGTCCACTTTCGTGTT
GGCAGAGTGCTGTGTTTTTAATAAACAGTCGCAGCCACCTATTCTCTGCGACCCT
CCGGGGCTTACGGAGCAAGTCCTTAACCTTAGAGGGCATACCTTCTCCCGAAGTT
ACGGTATCAATTTGCCGAGTTCCTTCTCCCGAGTTCTCTCAAGCGCCTTAGAATTC
TCATCCTGCCCACCTGTGTCGGTTTGCGGTACGGTTCGATTCAAACTGAAGCTTA
GTGGCTTTTCCTGGAAGCGTGGTATCGGTTGCTTCGTGTCCGTAGACACTCGTCA
TCACTTCTCGGTGTTAAGAAAACCCGGATTTGCCTAAGTCTTCCACCTACCGGCT
TAAACAAGCTATTCCAACAGCTTGCCAACCTAACCTTCTCCGTCCCCACATCGCA
TTTGAATCAAGTACAGGAATATTAACCTGTTTCCCATCGACTACGCATTTCTGCC
TCGCCTTAGGGGCCGACTCACCCTACGCCGATGAACGTTGCGTAGGAAACCTTG
GGCTTTGCGGCGAGCGGGCTTTTCACCCGCTTTATCGCTACTCATGTCAACATTC
GCACTTCTGATACCTCCAGCACACTTTACAATGCACCTTCATCAGCCTACAGAAC
GCTCCCCTACCATGCCGGTAAACCGGCATACGCAGCTTGGGTTATAGATTTGAGC
CCCGTTACATCTTCCGCGCAGGACGACTCGACCAGTGAGCTATTACGCTTTCTTT
AAATGATGGCTGCTTCTAAGCCAACATCCTGGCTGTCTGTGCCTTCCCACTTCGTT
TACCACTTAATCTATCATTTGGGACCTTAGCCGGCGGTCTGGGTTGTTTCCCTCTT
GACAACGGACGTTAGCACCCGCTGTCTGTCTCCCGAGGAACCACTTGATGGTATT
CTGAGTTTGCCATGGGTTGGTAAGTTGCAATAACCCCCTAGCCATAACAGTGCTT
TACCCCCATCAGTGTCTTGCTCGAGGCACTACCTAAATAGTTTTCGGGGAGAACC
AGCTATCTCCGAGTTTGTTTAGCCTTTCACCCCTACCCACAGCTCATCCCCGCATT
TTGCAACATGCGTGGGTTCGGTCCTCCAGTACCTGTTACGGCACCTTCAACCTGG
CCATGGATAGATCACTCGGTTTCGGGTCTACACCCAGCAACTCATCGCCCTATTA
AGACTCGGTTTCCCTACGCCTCCCCTATCCGGTTAAGCTCGCTACTGAATGTAAG
TCGTTGACCCATTATACAAAAGGTACGCAGTCACACCACTAGGGCGCTCCCACT
GTTTGTATGCATCAGGTTTCAGGTTCTGTTTCACTCCCCTCCCGGGGTTCTTTTCG
CCTTTCCCTCACGGTACTGGTTCACTATCGGTCGATGATGAGTATTTAGCCTTGG
AGGATGGTCCCCCCATATTCAGACAGGATTTCACGTGTCCCGCCCTACTTTTCGT
ACGCTTAGTACCGCTGTTGAGATTTCGAATACGGGACTGTCACCCGCTATGGTCA
AGCTTCCCAGCTTGTTCTTCTACCTCGACAGTTATTACGTACAGGCTCCTCCGCGT
TCGCTCGCCACTACTTGCGGAATCTCGGTTGATTTCTTTTCCTCCGGGTACTTAGA
TGGTTCAGTTCTCCGGGTTCGCTTCTCTAAGCCTATGTATTCAACTTAGGATACTG
CACAGAATGCAGTGGGTTTCCCCATTCGGACACCGCGGGATCATTGCTTTATTGC
CAGCTCCCCCGCGCTTTTCGCAGGCTTACACGTCCTTCGTCGCCTATCATCGCCA
AGGCATCCGCCTGATGCACTTATTCACTTGACTCTATCATTTCA

### 4. NGO_r06: NC_002946.2:c1263390-1261846

DNA (- strand): SEQ ID NO: 333

TGAACATAAGAGTTTGATCCTGGCTCAGATTGAACGCTGGCGGCATGCTTTACAC
ATGCAAGTCGGACGGCAGCACAGGGAAGCTTGCTTCTCGGGTGGCGAGTGGCGA
ACGGGTGAGTAACATATCGGAACGTACCGGGTAGCGGGGGATAACTGATCGAAA
GATCAGCTAATACCGCATACGTCTTGAGAGGGAAAGCAGGGGACCTTCGGGCCT
TGCGCTATCCGAGCGGCCGATATCTGATTAGCTGGTTGGCGGGGTAAAGGCCCA
CCAAGGCGACGATCAGTAGCGGGTCTGAGAGGATGATCCGCCACACTGGGACTG
AGACACGGCCCAGACTCCTACGGGAGGCAGCAGTGGGGAATTTTGGACAATGGG
CGCAAGCCTGATCCAGCCATGCCGCGTGTCTGAAGAAGGCCTTCGGGTTGTAAA
GGACTTTTGTCAGGGAAGAAAAGGCCGTTGCCAATATCGGCGGCCGATGACGGT
ACCTGAAGAATAAGCACCGGCTAACTACGTGCCAGCAGCCGCGGTAATACGTAG
GGTGCGAGCGTTAATCGGAATTACTGGGCGTAAAGCGGGCGCAGACGGTTACTT
AAGCAGGATGTGAAATCCCCGGGCTCAACCCGGGAACTGCGTTCTGAACTGGGT
GACTCGAGTGTGTCAGAGGGAGGTGGAATTCCACGTGTAGCAGTGAAATGCGTA
GAGATGTGGAGGAATACCGATGGCGAAGGCAGCCTCCTGGGATAACACTGACGT
TCATGTCCGAAAGCGTGGGTAGCAAACAGGATTAGATACCCTGGTAGTCCACGC
CCTAAACGATGTCAATTAGCTGTTGGGCAACTTGATTGCTTGGTAGCGTAGCTAA
CGCGTGAAATTGACCGCCTGGGGAGTACGGTCGCAAGATTAAAACTCAAAGGAA
TTGACGGGGACCCGCACAAGCGGTGGATGATGTGGATTAATTCGATGCAACGCG
AAGAACCTTACCTGGTTTTGACATGTGCGGAATCCTCCGGAGACGGAGGAGTGC
CTTCGGGAGCCGTAACACAGGTGCTGCATGGCTGTCGTCAGCTCGTGTCGTGAGA
TGTTGGGTTAAGTCCCGCAACGAGCGCAACCCTTGTCATTAGTTGCCATCATTCG
GTTGGGCACTCTAATGAGACTGCCGGTGACAAGCCGGAGGAAGGTGGGGATGAC
GTCAAGTCCTCATGGCCCTTATGACCAGGGCTTCACACGTCATACAATGGTCGGT
ACAGAGGGTAGCCAAGCCGCGAGGCGGAGCCAATCTCACAAAACCGATCGTAG
TCCGGATTGCACTCTGCAACTCGAGTGCATGAAGTCGGAATCGCTAGTAATCGCA
GGTCAGCATACTGCGGTGAATACGTTCCCGGGTCTTGTACACACCGCCCGTCACA
CCATGGGAGTGGGGGATACCAGAAGTAGGTAGGGTAACCGCAAGGAGTCCGCTT
ACCACGGTATGCTTCATGACTGGGGTGAAGTCGTAACAAGGTAGCCGTAGGGGA
ACCTGCGGCTGGATCACCTCCTTTCTA

RNA: SEQ ID NO: 334

UGAACAUAAGAGUUUGAUCCUGGCUCAGAUUGAACGCUGGCGGCAUGCUUUA
CACAUGCAAGUCGGACGGCAGCACAGGGAAGCUUGCUUCUCGGGUGGCGAGU
GGCGAACGGGUGAGUAACAUAUCGGAACGUACCGGGUAGCGGGGGAUAACUG
AUCGAAAGAUCAGCUAAUACCGCAUACGUCUUGAGAGGGAAAGCAGGGGACC
UUCGGGCCUUGCGCUAUCCGAGCGGCCGAUAUCUGAUUAGCUGGUUGGCGGG
GUAAAGGCCCACCAAGGCGACGAUCAGUAGCGGGUCUGAGAGGAUGAUCCGC
CACACUGGGACUGAGACACGGCCCAGACUCCUACGGGAGGCAGCAGUGGGGAA
UUUUGGACAAUGGGCGCAAGCCUGAUCCAGCCAUGCCGCGUGUCUGAAGAAG
GCCUUCGGGUUGUAAAGGACUUUUGUCAGGGAAGAAAAGGCCGUUGCCAAUA
UCGGCGGCCGAUGACGGUACCUGAAGAAUAAGCACCGGCUAACUACGUGCCAG
CAGCCGCGGUAAUACGUAGGGUGCGAGCGUUAAUCGGAAUUACUGGGCGUAA
AGCGGGCGCAGACGGUUACUUAAGCAGGAUGUGAAAUCCCCGGGCUCAACCCG
GGAACUGCGUUCUGAACUGGGUGACUCGAGUGUGUCAGAGGGAGGUGGAAUU
CCACGUGUAGCAGUGAAAUGCGUAGAGAUGUGGAGGAAUACCGAUGGCGAAG
GCAGCCUCCUGGGAUAACACUGACGUUCAUGUCCGAAAGCGUGGGUAGCAAA
CAGGAUUAGAUACCCUGGUAGUCCACGCCCUAAACGAUGUCAAUUAGCUGUU
GGGCAACUUGAUUGCUUGGUAGCGUAGCUAACGCGUGAAAUUGACCGCCUGG
GGAGUACGGUCGCAAGAUUAAAACUCAAAGGAAUUGACGGGGACCCGCACAA
GCGGUGGAUGAUGUGGAUUAAUUCGAUGCAACGCGAAGAACCUUACCUGGUU
UUGACAUGUGCGGAAUCCUCCGGAGACGGAGGAGUGCCUUCGGGAGCCGUAA
CACAGGUGCUGCAUGGCUGUCGUCAGCUCGUGUCGUGAGAUGUUGGGUUAAG
UCCCGCAACGAGCGCAACCCUUGUCAUUAGUUGCCAUCAUUCGGUUGGGCACU
CUAAUGAGACUGCCGGUGACAAGCCGGAGGAAGGUGGGGAUGACGUCAAGUC
CUCAUGGCCCUUAUGACCAGGGCUUCACACGUCAUACAAUGGUCGGUACAGAG
GGUAGCCAAGCCGCGAGGCGGAGCCAAUCUCACAAAACCGAUCGUAGUCCGGA
UUGCACUCUGCAACUCGAGUGCAUGAAGUCGGAAUCGCUAGUAAUCGCAGGU
CAGCAUACUGCGGUGAAUACGUUCCCGGGUCUUGUACACACCGCCCGUCACAC
CAUGGGAGUGGGGGAUACCAGAAGUAGGUAGGGUAACCGCAAGGAGUCCGCU
UACCACGGUAUGCUUCAUGACUGGGGUGAAGUCGUAACAAGGUAGCCGUAGG
GGAACCUGCGGCUGGAUCACCUCCUUUCUA

cDNA: SEQ ID NO: 335

TAGAAAGGAGGTGATCCAGCCGCAGGTTCCCCTACGGCTACCTTGTTACGACTTC
ACCCCAGTCATGAAGCATACCGTGGTAAGCGGACTCCTTGCGGTTACCCTACCTA
CTTCTGGTATCCCCCACTCCCATGGTGTGACGGGCGGTGTGTACAAGACCCGGGA
ACGTATTCACCGCAGTATGCTGACCTGCGATTACTAGCGATTCCGACTTCATGCA
CTCGAGTTGCAGAGTGCAATCCGGACTACGATCGGTTTTGTGAGATTGGCTCCGC
CTCGCGGCTTGGCTACCCTCTGTACCGACCATTGTATGACGTGTGAAGCCCTGGT
CATAAGGGCCATGAGGACTTGACGTCATCCCCACCTTCCTCCGGCTTGTCACCGG
CAGTCTCATTAGAGTGCCCAACCGAATGATGGCAACTAATGACAAGGGTTGCGC
TCGTTGCGGGACTTAACCCAACATCTCACGACACGAGCTGACGACAGCCATGCA
GCACCTGTGTTACGGCTCCCGAAGGCACTCCTCCGTCTCCGGAGGATTCCGCACA
TGTCAAAACCAGGTAAGGTTCTTCGCGTTGCATCGAATTAATCCACATCATCCAC
CGCTTGTGCGGGTCCCCGTCAATTCCTTTGAGTTTTAATCTTGCGACCGTACTCCC
CAGGCGGTCAATTTCACGCGTTAGCTACGCTACCAAGCAATCAAGTTGCCCAAC
AGCTAATTGACATCGTTTAGGGCGTGGACTACCAGGGTATCTAATCCTGTTTGCT
ACCCACGCTTTCGGACATGAACGTCAGTGTTATCCCAGGAGGCTGCCTTCGCCAT
CGGTATTCCTCCACATCTCTACGCATTTCACTGCTACACGTGGAATTCCACCTCCC
TCTGACACACTCGAGTCACCCAGTTCAGAACGCAGTTCCCGGGTTGAGCCCGGG
GATTTCACATCCTGCTTAAGTAACCGTCTGCGCCCGCTTTACGCCCAGTAATTCC
GATTAACGCTCGCACCCTACGTATTACCGCGGCTGCTGGCACGTAGTTAGCCGGT
GCTTATTCTTCAGGTACCGTCATCGGCCGCCGATATTGGCAACGGCCTTTTCTTCC
CTGACAAAAGTCCTTTACAACCCGAAGGCCTTCTTCAGACACGCGGCATGGCTG
GATCAGGCTTGCGCCCATTGTCCAAAATTCCCCACTGCTGCCTCCCGTAGGAGTC
TGGGCCGTGTCTCAGTCCCAGTGTGGCGGATCATCCTCTCAGACCCGCTACTGAT
CGTCGCCTTGGTGGGCCTTTACCCCGCCAACCAGCTAATCAGATATCGGCCGCTC
GGATAGCGCAAGGCCCGAAGGTCCCCTGCTTTCCCTCTCAAGACGTATGCGGTAT
TAGCTGATCTTTCGATCAGTTATCCCCCGCTACCCGGTACGTTCCGATATGTTACT
CACCCGTTCGCCACTCGCCACCCGAGAAGCAAGCTTCCCTGTGCTGCCGTCCGAC
TTGCATGTGTAAAGCATGCCGCCAGCGTTCAATCTGAGCCAGGATCAAACTCTTA
TGTTCA

### 5. NGO_r08: NC_002946.2:c1652830-1649928

DNA (- strand): SEQ ID NO: 336

TGAAATGATAGAGTCAAGTGAATAAGTGCATCAGGCGGATGCCTTGGCGATGAT
AGGCGACGAAGGACGTGTAAGCCTGCGAAAAGCGCGGGGGAGCTGGCAATAAA
GCAATGATCCCGCGGTGTCCGAATGGGGAAACCCACTGCATTCTGTGCAGTATC
CTAAGTTGAATACATAGGCTTAGAGAAGCGAACCCGGAGAACTGAACCATCTAA
GTACCCGGAGGAAAAGAAATCAACCGAGATTCCGCAAGTAGTGGCGAGCGAAC
GCGGAGGAGCCTGTACGTAATAACTGTCGAGGTAGAAGAACAAGCTGGGAAGCT
TGACCATAGCGGGTGACAGTCCCGTATTCGAAATCTCAACAGCGGTACTAAGCG
TACGAAAAGTAGGGCGGGACACGTGAAATCCTGTCTGAATATGGGGGGACCATC
CTCCAAGGCTAAATACTCATCATCGACCGATAGTGAACCAGTACCGTGAGGGAA
AGGCGAAAAGAACCCCGGGAGGGGAGTGAAACAGAACCTGAAACCTGATGCAT
ACAAACAGTGGGAGCGCCCTAGTGGTGTGACTGCGTACCTTTTGTATAATGGGTC
AACGACTTACATTCAGTAGCGAGCTTAACCGGATAGGGGAGGCGTAGGGAAACC
GAGTCTTAATAGGGCGATGAGTTGCTGGGTGTAGACCCGAAACCGAGTGATCTA
TCCATGGCCAGGTTGAAGGTGCCGTAACAGGTACTGGAGGACCGAACCCACGCA
TGTTGCAAAATGCGGGGATGAGCTGTGGGTAGGGGTGAAAGGCTAAACAAACTC
GGAGATAGCTGGTTCTCCCCGAAAACTATTTAGGTAGTGCCTCGAGCAAGACAC
TGATGGGGGTAAAGCACTGTTATGGCTAGGGGGTTATTGCAACTTACCAACCCAT
GGCAAACTCAGAATACCATCAAGTGGTTCCTCGGGAGACAGACAGCGGGTGCTA
ACGTCCGTTGTCAAGAGGGAAACAACCCAGACCGCCGGCTAAGGTCCCAAATGA
TAGATTAAGTGGTAAACGAAGTGGGAAGGCACAGACAGCCAGGATGTTGGCTTA
GAAGCAGCCATCATTTAAAGAAAGCGTAATAGCTCACTGGTCGAGTCGTCCTGC
GCGGAAGATGTAACGGGGCTCAAATCTATAACCGAAGCTGCGGATGCCGGTTTA
CCGGCATGGTAGGGGAGCGTTCTGTAGGCTGATGAAGGTGCATTGTAAAGTGTG
CTGGAGGTATCAGAAGTGCGAATGTTGACATGAGTAGCGATAAAGCGGGTGAAA
AGCCCGCTCGCCGAAAGCCCAAGGTTTCCTACGCAACGTTCATCGGCGTAGGGT
GAGTCGGCCCCTAAGGCGAGGCAGAAATGCGTAGTCGATGGGAAACAGGTTAAT
ATTCCTGTACTTGATTCAAATGCGATGTGGGGACGGAGAAGGTTAGGTTGGCAA
GCTGTTGGAATAGCTTGTTTAAGCCGGTAGGTGGAAGACTTAGGCAAATCCGGG
TTTTCTTAACACCGAGAAGTGATGACGAGTGTCTACGGACACGAAGCAACCGAT
ACCACGCTTCCAGGAAAAGCCACTAAGCTTCAGTTTGAATCGAACCGTACCGCA
AACCGACACAGGTGGGCAGGATGAGAATTCTAAGGCGCTTGAGAGAACTCGGG
AGAAGGAACTCGGCAAATTGATACCGTAACTTCGGGAGAAGGTATGCCCTCTAA
GGTTAAGGACTTGCTCCGTAAGCCCCGGAGGGTCGCAGAGAATAGGTGGCTGCG
ACTGTTTATTAAAAACACAGCACTCTGCCAACACGAAAGTGGACGTATAGGGTG
TGACGCCTGCCCGGTGCCGGAAGGTTAATTGAAGATGTGCAAGCATCGGATCGA
AGCCCCGGTAAACGGCGGCCGTAACTATAACGGTCCTAAGGTAGCGAAATTCCT
TGTCGGGTAAGTTCCGACCCGCACGAATGGCGTAACGATGGCCACACTGTCTCCT
CCCGAGACTCAGCGAAGTTGAAGTGGTTGTGAAGATGCAATCTACCCGCTGCTA
GACGGAAAGACCCCGTGAACCTTTACTGTAGCTTTGCATTGGACTTTGAAGTCAC
TTGTGTAGGATAGGTGGGAGGCTTGGAAGCAGAGACGCCAGTCTCTGTGGAGTC
GTCCTTGAAATACCACCCTGGTGTCTTTGAGGTTCTAACCCAGACCCGTCATCCG
GGTCGGGGACCGTGCATGGTAGGCAGTTTGACTGGGGCGGTCTCCTCCCAAAGC
GTAACGGAGGAGTTCGAAGGTTACCTAGGTCCGGTCGGAAATCGGACTGATAGT
GCAATGGCAAAAGGTAGCTTAACTGCGAGACCGACAAGTCGGGCAGGTGCGAA
AGCAGGACATAGTGATCCGGTGGTTCTGTATGGAAGGGCCATCGCTCAACGGAT
AAAAGGTACTCCGGGGATAACAGGCTGATTCCGCCCAAGAGTTCATATCGACGG
CGGAGTTTGGCACCTCGATGTCGGCTCATCACATCCTGGGGCTGTAGTCGGTCCC
AAGGGTATGGCTGTTCGCCATTTAAAGTGGTACGTGAGCTGGGTTTAAAACGTCG
TGAGACAGTTTGGTCCCTATCTGCAGTGGGCGTTGGAAGTTTGACGGGGGCTGCT
CCTAGTACGAGAGGACCGGAGTGGACGAACCTCTGGTGTACCGGTTGTAACGCC
AGTTGCATAGCCGGGTAGCTAAGTTCGGAAGAGATAAGCGCTGAAAGCATCTAA
GCGCGAAACTCGCCTGAAGATGAGACTTCCCTTGCGGTTTAACCGCACTAAAGG
GTCGTTCGAGACCAGGACGTTGATAGGTGGGGTGTGGAAGCGCGGTAACGCGTG
AAGCTAACCCATACTAATTGCCCGTGAGGCTTGACTCT

RNA: SEQ ID NO: 337

UGAAAUGAUAGAGUCAAGUGAAUAAGUGCAUCAGGCGGAUGCCUUGGCGAUG
AUAGGCGACGAAGGACGUGUAAGCCUGCGAAAAGCGCGGGGGAGCUGGCAAU
AAAGCAAUGAUCCCGCGGUGUCCGAAUGGGGAAACCCACUGCAUUCUGUGCA
GUAUCCUAAGUUGAAUACAUAGGCUUAGAGAAGCGAACCCGGAGAACUGAAC
CAUCUAAGUACCCGGAGGAAAAGAAAUCAACCGAGAUUCCGCAAGUAGUGGC
GAGCGAACGCGGAGGAGCCUGUACGUAAUAACUGUCGAGGUAGAAGAACAAG
CUGGGAAGCUUGACCAUAGCGGGUGACAGUCCCGUAUUCGAAAUCUCAACAG
CGGUACUAAGCGUACGAAAAGUAGGGCGGGACACGUGAAAUCCUGUCUGAAU
AUGGGGGGACCAUCCUCCAAGGCUAAAUACUCAUCAUCGACCGAUAGUGAACC
AGUACCGUGAGGGAAAGGCGAAAAGAACCCCGGGAGGGGAGUGAAACAGAAC
CUGAAACCUGAUGCAUACAAACAGUGGGAGCGCCCUAGUGGUGUGACUGCGU
ACCUUUUGUAUAAUGGGUCAACGACUUACAUUCAGUAGCGAGCUUAACCGGA
UAGGGGAGGCGUAGGGAAACCGAGUCUUAAUAGGGCGAUGAGUUGCUGGGUG
UAGACCCGAAACCGAGUGAUCUAUCCAUGGCCAGGUUGAAGGUGCCGUAACA
GGUACUGGAGGACCGAACCCACGCAUGUUGCAAAAUGCGGGGAUGAGCUGUG
GGUAGGGGUGAAAGGCUAAACAAACUCGGAGAUAGCUGGUUCUCCCCGAAAA
CUAUUUAGGUAGUGCCUCGAGCAAGACACUGAUGGGGGUAAAGCACUGUUAU
GGCUAGGGGGUUAUUGCAACUUACCAACCCAUGGCAAACUCAGAAUACCAUC
AAGUGGUUCCUCGGGAGACAGACAGCGGGUGCUAACGUCCGUUGUCAAGAGG
GAAACAACCCAGACCGCCGGCUAAGGUCCCAAAUGAUAGAUUAAGUGGUAAA
CGAAGUGGGAAGGCACAGACAGCCAGGAUGUUGGCUUAGAAGCAGCCAUCAU
UUAAAGAAAGCGUAAUAGCUCACUGGUCGAGUCGUCCUGCGCGGAAGAUGUA
ACGGGGCUCAAAUCUAUAACCGAAGCUGCGGAUGCCGGUUUACCGGCAUGGU
AGGGGAGCGUUCUGUAGGCUGAUGAAGGUGCAUUGUAAAGUGUGCUGGAGGU
AUCAGAAGUGCGAAUGUUGACAUGAGUAGCGAUAAAGCGGGUGAAAAGCCCG
CUCGCCGAAAGCCCAAGGUUUCCUACGCAACGUUCAUCGGCGUAGGGUGAGUC
GGCCCCUAAGGCGAGGCAGAAAUGCGUAGUCGAUGGGAAACAGGUUAAUAUU
CCUGUACUUGAUUCAAAUGCGAUGUGGGGACGGAGAAGGUUAGGUUGGCAAG
CUGUUGGAAUAGCUUGUUUAAGCCGGUAGGUGGAAGACUUAGGCAAAUCCGG
GUUUUCUUAACACCGAGAAGUGAUGACGAGUGUCUACGGACACGAAGCAACC
GAUACCACGCUUCCAGGAAAAGCCACUAAGCUUCAGUUUGAAUCGAACCGUAC
CGCAAACCGACACAGGUGGGCAGGAUGAGAAUUCUAAGGCGCUUGAGAGAAC
UCGGGAGAAGGAACUCGGCAAAUUGAUACCGUAACUUCGGGAGAAGGUAUGC
CCUCUAAGGUUAAGGACUUGCUCCGUAAGCCCCGGAGGGUCGCAGAGAAUAG
GUGGCUGCGACUGUUUAUUAAAAACACAGCACUCUGCCAACACGAAAGUGGA
CGUAUAGGGUGUGACGCCUGCCCGGUGCCGGAAGGUUAAUUGAAGAUGUGCA
AGCAUCGGAUCGAAGCCCCGGUAAACGGCGGCCGUAACUAUAACGGUCCUAAG
GUAGCGAAAUUCCUUGUCGGGUAAGUUCCGACCCGCACGAAUGGCGUAACGA
UGGCCACACUGUCUCCUCCCGAGACUCAGCGAAGUUGAAGUGGUUGUGAAGA
UGCAAUCUACCCGCUGCUAGACGGAAAGACCCCGUGAACCUUUACUGUAGCUU
UGCAUUGGACUUUGAAGUCACUUGUGUAGGAUAGGUGGGAGGCUUGGAAGCA
GAGACGCCAGUCUCUGUGGAGUCGUCCUUGAAAUACCACCCUGGUGUCUUUG
AGGUUCUAACCCAGACCCGUCAUCCGGGUCGGGGACCGUGCAUGGUAGGCAGU
UUGACUGGGGCGGUCUCCUCCCAAAGCGUAACGGAGGAGUUCGAAGGUUACC
UAGGUCCGGUCGGAAAUCGGACUGAUAGUGCAAUGGCAAAAGGUAGCUUAAC
UGCGAGACCGACAAGUCGGGCAGGUGCGAAAGCAGGACAUAGUGAUCCGGUG
GUUCUGUAUGGAAGGGCCAUCGCUCAACGGAUAAAAGGUACUCCGGGGAUAA
CAGGCUGAUUCCGCCCAAGAGUUCAUAUCGACGGCGGAGUUUGGCACCUCGAU
GUCGGCUCAUCACAUCCUGGGGCUGUAGUCGGUCCCAAGGGUAUGGCUGUUC
GCCAUUUAAAGUGGUACGUGAGCUGGGUUUAAAACGUCGUGAGACAGUUUGG
UCCCUAUCUGCAGUGGGCGUUGGAAGUUUGACGGGGGCUGCUCCUAGUACGA
GAGGACCGGAGUGGACGAACCUCUGGUGUACCGGUUGUAACGCCAGUUGCAU
AGCCGGGUAGCUAAGUUCGGAAGAGAUAAGCGCUGAAAGCAUCUAAGCGCGA
AACUCGCCUGAAGAUGAGACUUCCCUUGCGGUUUAACCGCACUAAAGGGUCG
UUCGAGACCAGGACGUUGAUAGGUGGGGUGUGGAAGCGCGGUAACGCGUGAA
GCUAACCCAUACUAAUUGCCCGUGAGGCUUGACUCU

cDNA: SEQ ID NO: 338

AGAGTCAAGCCTCACGGGCAATTAGTATGGGTTAGCTTCACGCGTTACCGCGCTT
CCACACCCCACCTATCAACGTCCTGGTCTCGAACGACCCTTTAGTGCGGTTAAAC
CGCAAGGGAAGTCTCATCTTCAGGCGAGTTTCGCGCTTAGATGCTTTCAGCGCTT
ATCTCTTCCGAACTTAGCTACCCGGCTATGCAACTGGCGTTACAACCGGTACACC
AGAGGTTCGTCCACTCCGGTCCTCTCGTACTAGGAGCAGCCCCCGTCAAACTTCC
AACGCCCACTGCAGATAGGGACCAAACTGTCTCACGACGTTTTAAACCCAGCTC
ACGTACCACTTTAAATGGCGAACAGCCATACCCTTGGGACCGACTACAGCCCCA
GGATGTGATGAGCCGACATCGAGGTGCCAAACTCCGCCGTCGATATGAACTCTT
GGGCGGAATCAGCCTGTTATCCCCGGAGTACCTTTTATCCGTTGAGCGATGGCCC
TTCCATACAGAACCACCGGATCACTATGTCCTGCTTTCGCACCTGCCCGACTTGT
CGGTCTCGCAGTTAAGCTACCTTTTGCCATTGCACTATCAGTCCGATTTCCGACC
GGACCTAGGTAACCTTCGAACTCCTCCGTTACGCTTTGGGAGGAGACCGCCCCA
GTCAAACTGCCTACCATGCACGGTCCCCGACCCGGATGACGGGTCTGGGTTAGA
ACCTCAAAGACACCAGGGTGGTATTTCAAGGACGACTCCACAGAGACTGGCGTC
TCTGCTTCCAAGCCTCCCACCTATCCTACACAAGTGACTTCAAAGTCCAATGCAA
AGCTACAGTAAAGGTTCACGGGGTCTTTCCGTCTAGCAGCGGGTAGATTGCATCT
TCACAACCACTTCAACTTCGCTGAGTCTCGGGAGGAGACAGTGTGGCCATCGTTA
CGCCATTCGTGCGGGTCGGAACTTACCCGACAAGGAATTTCGCTACCTTAGGACC
GTTATAGTTACGGCCGCCGTTTACCGGGGCTTCGATCCGATGCTTGCACATCTTC
AATTAACCTTCCGGCACCGGGCAGGCGTCACACCCTATACGTCCACTTTCGTGTT
GGCAGAGTGCTGTGTTTTTAATAAACAGTCGCAGCCACCTATTCTCTGCGACCCT
CCGGGGCTTACGGAGCAAGTCCTTAACCTTAGAGGGCATACCTTCTCCCGAAGTT
ACGGTATCAATTTGCCGAGTTCCTTCTCCCGAGTTCTCTCAAGCGCCTTAGAATTC
TCATCCTGCCCACCTGTGTCGGTTTGCGGTACGGTTCGATTCAAACTGAAGCTTA
GTGGCTTTTCCTGGAAGCGTGGTATCGGTTGCTTCGTGTCCGTAGACACTCGTCA
TCACTTCTCGGTGTTAAGAAAACCCGGATTTGCCTAAGTCTTCCACCTACCGGCT
TAAACAAGCTATTCCAACAGCTTGCCAACCTAACCTTCTCCGTCCCCACATCGCA
TTTGAATCAAGTACAGGAATATTAACCTGTTTCCCATCGACTACGCATTTCTGCC
TCGCCTTAGGGGCCGACTCACCCTACGCCGATGAACGTTGCGTAGGAAACCTTG
GGCTTTCGGCGAGCGGGCTTTTCACCCGCTTTATCGCTACTCATGTCAACATTCG
CACTTCTGATACCTCCAGCACACTTTACAATGCACCTTCATCAGCCTACAGAACG
CTCCCCTACCATGCCGGTAAACCGGCATCCGCAGCTTCGGTTATAGATTTGAGCC
CCGTTACATCTTCCGCGCAGGACGACTCGACCAGTGAGCTATTACGCTTTCTTTA
AATGATGGCTGCTTCTAAGCCAACATCCTGGCTGTCTGTGCCTTCCCACTTCGTTT
ACCACTTAATCTATCATTTGGGACCTTAGCCGGCGGTCTGGGTTGTTTCCCTCTTG
ACAACGGACGTTAGCACCCGCTGTCTGTCTCCCGAGGAACCACTTGATGGTATTC
TGAGTTTGCCATGGGTTGGTAAGTTGCAATAACCCCCTAGCCATAACAGTGCTTT
ACCCCCATCAGTGTCTTGCTCGAGGCACTACCTAAATAGTTTTCGGGGAGAACCA
GCTATCTCCGAGTTTGTTTAGCCTTTCACCCCTACCCACAGCTCATCCCCGCATTT
TGCAACATGCGTGGGTTCGGTCCTCCAGTACCTGTTACGGCACCTTCAACCTGGC
CATGGATAGATCACTCGGTTTCGGGTCTACACCCAGCAACTCATCGCCCTATTAA
GACTCGGTTTCCCTACGCCTCCCCTATCCGGTTAAGCTCGCTACTGAATGTAAGT
CGTTGACCCATTATACAAAAGGTACGCAGTCACACCACTAGGGCGCTCCCACTG
TTTGTATGCATCAGGTTTCAGGTTCTGTTTCACTCCCCTCCCGGGGTTCTTTTCGC
CTTTCCCTCACGGTACTGGTTCACTATCGGTCGATGATGAGTATTTAGCCTTGGA
GGATGGTCCCCCCATATTCAGACAGGATTTCACGTGTCCCGCCCTACTTTTCGTA
CGCTTAGTACCGCTGTTGAGATTTCGAATACGGGACTGTCACCCGCTATGGTCAA
GCTTCCCAGCTTGTTCTTCTACCTCGACAGTTATTACGTACAGGCTCCTCCGCGTT
CGCTCGCCACTACTTGCGGAATCTCGGTTGATTTCTTTTCCTCCGGGTACTTAGAT
GGTTCAGTTCTCCGGGTTCGCTTCTCTAAGCCTATGTATTCAACTTAGGATACTGC
ACAGAATGCAGTGGGTTTCCCCATTCGGACACCGCGGGATCATTGCTTTATTGCC
AGCTCCCCCGCGCTTTTCGCAGGCTTACACGTCCTTCGTCGCCTATCATCGCCAA
GGCATCCGCCTGATGCACTTATTCACTTGACTCTATCATTTCA

### 6. NGO_r09: NC_002946.2:c1654965-1653421

DNA (- strand): SEQ ID NO: 339

TGAACATAAGAGTTTGATCCTGGCTCAGATTGAACGCTGGCGGCATGCTTTACAC
ATGCAAGTCGGACGGCAGCACAGGGAAGCTTGCTTCTCGGGTGGCGAGTGGCGA
ACGGGTGAGTAACATATCGGAACGTACCGGGTAGCGGGGGATAACTGATCGAAA
GATCAGCTAATACCGCATACGTCTTGAGAGGGAAAGCAGGGGACCTTCGGGCCT
TGCGCTATCCGAGCGGCCGATATCTGATTAGCTGGTTGGCGGGGTAAAGGCCCA
CCAAGGCGACGATCAGTAGCGGGTCTGAGAGGATGATCCGCCACACTGGGACTG
AGACACGGCCCAGACTCCTACGGGAGGCAGCAGTGGGGAATTTTGGACAATGGG
CGCAAGCCTGATCCAGCCATGCCGCGTGTCTGAAGAAGGCCTTCGGGTTGTAAA
GGACTTTTGTCAGGGAAGAAAAGGCCGTTGCCAATATCGGCGGCCGATGACGGT
ACCTGAAGAATAAGCACCGGCTAACTACGTGCCAGCAGCCGCGGTAATACGTAG
GGTGCGAGCGTTAATCGGAATTACTGGGCGTAAAGCGGGCGCAGACGGTTACTT
AAGCAGGATGTGAAATCCCCGGGCTCAACCCGGGAACTGCGTTCTGAACTGGGT
GACTCGAGTGTGTCAGAGGGAGGTGGAATTCCACGTGTAGCAGTGAAATGCGTA
GAGATGTGGAGGAATACCGATGGCGAAGGCAGCCTCCTGGGATAACACTGACGT
TCATGTCCGAAAGCGTGGGTAGCAAACAGGATTAGATACCCTGGTAGTCCACGC
CCTAAACGATGTCAATTAGCTGTTGGGCAACTTGATTGCTTGGTAGCGTAGCTAA
CGCGTGAAATTGACCGCCTGGGGAGTACGGTCGCAAGATTAAAACTCAAAGGAA
TTGACGGGGACCCGCACAAGCGGTGGATGATGTGGATTAATTCGATGCAACGCG
AAGAACCTTACCTGGTTTTGACATGTGCGGAATCCTCCGGAGACGGAGGAGTGC
CTTCGGGAGCCGTAACACAGGTGCTGCATGGCTGTCGTCAGCTCGTGTCGTGAGA
TGTTGGGTTAAGTCCCGCAACGAGCGCAACCCTTGTCATTAGTTGCCATCATTCG
GTTGGGCACTCTAATGAGACTGCCGGTGACAAGCCGGAGGAAGGTGGGGATGAC
GTCAAGTCCTCATGGCCCTTATGACCAGGGCTTCACACGTCATACAATGGTCGGT
ACAGAGGGTAGCCAAGCCGCGAGGCGGAGCCAATCTCACAAAACCGATCGTAG
TCCGGATTGCACTCTGCAACTCGAGTGCATGAAGTCGGAATCGCTAGTAATCGCA
GGTCAGCATACTGCGGTGAATACGTTCCCGGGTCTTGTACACACCGCCCGTCACA
CCATGGGAGTGGGGGATACCAGAAGTAGGTAGGGTAACCGCAAGGAGTCCGCTT
ACCACGGTATGCTTCATGACTGGGGTGAAGTCGTAACAAGGTAGCCGTAGGGGA
ACCTGCGGCTGGATCACCTCCTTTCTA

RNA: SEQ ID NO: 340

UGAACAUAAGAGUUUGAUCCUGGCUCAGAUUGAACGCUGGCGGCAUGCUUUA
CACAUGCAAGUCGGACGGCAGCACAGGGAAGCUUGCUUCUCGGGUGGCGAGU
GGCGAACGGGUGAGUAACAUAUCGGAACGUACCGGGUAGCGGGGGAUAACUG
AUCGAAAGAUCAGCUAAUACCGCAUACGUCUUGAGAGGGAAAGCAGGGGACC
UUCGGGCCUUGCGCUAUCCGAGCGGCCGAUAUCUGAUUAGCUGGUUGGCGGG
GUAAAGGCCCACCAAGGCGACGAUCAGUAGCGGGUCUGAGAGGAUGAUCCGC
CACACUGGGACUGAGACACGGCCCAGACUCCUACGGGAGGCAGCAGUGGGGAA
UUUUGGACAAUGGGCGCAAGCCUGAUCCAGCCAUGCCGCGUGUCUGAAGAAG
GCCUUCGGGUUGUAAAGGACUUUUGUCAGGGAAGAAAAGGCCGUUGCCAAUA
UCGGCGGCCGAUGACGGUACCUGAAGAAUAAGCACCGGCUAACUACGUGCCAG
CAGCCGCGGUAAUACGUAGGGUGCGAGCGUUAAUCGGAAUUACUGGGCGUAA
AGCGGGCGCAGACGGUUACUUAAGCAGGAUGUGAAAUCCCCGGGCUCAACCCG
GGAACUGCGUUCUGAACUGGGUGACUCGAGUGUGUCAGAGGGAGGUGGAAUU
CCACGUGUAGCAGUGAAAUGCGUAGAGAUGUGGAGGAAUACCGAUGGCGAAG
GCAGCCUCCUGGGAUAACACUGACGUUCAUGUCCGAAAGCGUGGGUAGCAAA
CAGGAUUAGAUACCCUGGUAGUCCACGCCCUAAACGAUGUCAAUUAGCUGUU
GGGCAACUUGAUUGCUUGGUAGCGUAGCUAACGCGUGAAAUUGACCGCCUGG
GGAGUACGGUCGCAAGAUUAAAACUCAAAGGAAUUGACGGGGACCCGCACAA
GCGGUGGAUGAUGUGGAUUAAUUCGAUGCAACGCGAAGAACCUUACCUGGUU
UUGACAUGUGCGGAAUCCUCCGGAGACGGAGGAGUGCCUUCGGGAGCCGUAA
CACAGGUGCUGCAUGGCUGUCGUCAGCUCGUGUCGUGAGAUGUUGGGUUAAG
UCCCGCAACGAGCGCAACCCUUGUCAUUAGUUGCCAUCAUUCGGUUGGGCACU
CUAAUGAGACUGCCGGUGACAAGCCGGAGGAAGGUGGGGAUGACGUCAAGUC
CUCAUGGCCCUUAUGACCAGGGCUUCACACGUCAUACAAUGGUCGGUACAGAG
GGUAGCCAAGCCGCGAGGCGGAGCCAAUCUCACAAAACCGAUCGUAGUCCGGA
UUGCACUCUGCAACUCGAGUGCAUGAAGUCGGAAUCGCUAGUAAUCGCAGGU
CAGCAUACUGCGGUGAAUACGUUCCCGGGUCUUGUACACACCGCCCGUCACAC
CAUGGGAGUGGGGGAUACCAGAAGUAGGUAGGGUAACCGCAAGGAGUCCGCU
UACCACGGUAUGCUUCAUGACUGGGGUGAAGUCGUAACAAGGUAGCCGUAGG
GGAACCUGCGGCUGGAUCACCUCCUUUCUA

cDNA: SEQ ID NO: 341

TAGAAAGGAGGTGATCCAGCCGCAGGTTCCCCTACGGCTACCTTGTTACGACTTC
ACCCCAGTCATGAAGCATACCGTGGTAAGCGGACTCCTTGCGGTTACCCTACCTA
CTTCTGGTATCCCCCACTCCCATGGTGTGACGGGCGGTGTGTACAAGACCCGGGA
ACGTATTCACCGCAGTATGCTGACCTGCGATTACTAGCGATTCCGACTTCATGCA
CTCGAGTTGCAGAGTGCAATCCGGACTACGATCGGTTTTGTGAGATTGGCTCCGC
CTCGCGGCTTGGCTACCCTCTGTACCGACCATTGTATGACGTGTGAAGCCCTGGT
CATAAGGGCCATGAGGACTTGACGTCATCCCCACCTTCCTCCGGCTTGTCACCGG
CAGTCTCATTAGAGTGCCCAACCGAATGATGGCAACTAATGACAAGGGTTGCGC
TCGTTGCGGGACTTAACCCAACATCTCACGACACGAGCTGACGACAGCCATGCA
GCACCTGTGTTACGGCTCCCGAAGGCACTCCTCCGTCTCCGGAGGATTCCGCACA
TGTCAAAACCAGGTAAGGTTCTTCGCGTTGCATCGAATTAATCCACATCATCCAC
CGCTTGTGCGGGTCCCCGTCAATTCCTTTGAGTTTTAATCTTGCGACCGTACTCCC
CAGGCGGTCAATTTCACGCGTTAGCTACGCTACCAAGCAATCAAGTTGCCCAAC
AGCTAATTGACATCGTTTAGGGCGTGGACTACCAGGGTATCTAATCCTGTTTGCT
ACCCACGCTTTCGGACATGAACGTCAGTGTTATCCCAGGAGGCTGCCTTCGCCAT
CGGTATTCCTCCACATCTCTACGCATTTCACTGCTACACGTGGAATTCCACCTCCC
TCTGACACACTCGAGTCACCCAGTTCAGAACGCAGTTCCCGGGTTGAGCCCGGG
GATTTCACATCCTGCTTAAGTAACCGTCTGCGCCCGCTTTACGCCCAGTAATTCC
GATTAACGCTCGCACCCTACGTATTACCGCGGCTGCTGGCACGTAGTTAGCCGGT
GCTTATTCTTCAGGTACCGTCATCGGCCGCCGATATTGGCAACGGCCTTTTCTTCC
CTGACAAAAGTCCTTTACAACCCGAAGGCCTTCTTCAGACACGCGGCATGGCTG
GATCAGGCTTGCGCCCATTGTCCAAAATTCCCCACTGCTGCCTCCCGTAGGAGTC
TGGGCCGTGTCTCAGTCCCAGTGTGGCGGATCATCCTCTCAGACCCGCTACTGAT
CGTCGCCTTGGTGGGCCTTTACCCCGCCAACCAGCTAATCAGATATCGGCCGCTC
GGATAGCGCAAGGCCCGAAGGTCCCCTGCTTTCCCTCTCAAGACGTATGCGGTAT
TAGCTGATCTTTCGATCAGTTATCCCCCGCTACCCGGTACGTTCCGATATGTTACT
CACCCGTTCGCCACTCGCCACCCGAGAAGCAAGCTTCCCTGTGCTGCCGTCCGAC
TTGCATGTGTAAAGCATGCCGCCAGCGTTCAATCTGAGCCAGGATCAAACTCTTA
TGTTCA

### 7. NGO_r11: NC_002946.2:c1875982-1873080

DNA (- strand): SEQ ID NO: 342

TGAAATGATAGAGTCAAGTGAATAAGTGCATCAGGCGGATGCCTTGGCGATGAT
AGGCGACGAAGGACGTGTAAGCCTGCGAAAAGCGCGGGGGAGCTGGCAATAAA
GCAATGATCCCGCGGTGTCCGAATGGGGAAACCCACTGCATTCTGTGCAGTATC
CTAAGTTGAATACATAGGCTTAGAGAAGCGAACCCGGAGAACTGACCCATCTAA
GTACCCGGAGGAAAAGAAATCAACCGAGATTCCGCAAGTAGTGGCGAGCGAAC
GCGGAGGAGCCTGTACGTAATAACTGTCGAGGTAGAAGAACAAGCTGGGAAGCT
TGACCATAGCGGGTGACAGTCCCGTATTCGAAATCTCAACAGCGGTACTAAGCG
TACGAAAAGTAGGGCGGGACACGTGAAATCCTGTCTGAATATGGGGGGACCATC
CTCCAAGGCTAAATACTCATCATCGACCGATAGTGAACCAGTACCGTGAGGGAA
AGGCGAAAAGAACCCCGGGAGGGGAGTGAAACAGAACCTGAAACCTGATGCAT
ACAAACAGTGGGAGCGCCCTAGTGGTGTGACTGCGTACCTTTTGTATAATGGGTC
AACGACTTACATTCAGTAGCGAGCTTAACCGGATAGGGGAGGCGTAGGGAAACC
GAGTCTTAATAGGGCGATGAGTTGCTGGGTGTAGACCCGAAACCGAGTGATCTA
TCCATGGCCAGGTTGAAGGTGCCGTAACAGGTACTGGAGGACCGAACCCACGCA
TGTTGCAAAATGCGGGGATGAGCTGTGGGTAGGGGTGAAAGGCTAAACAAACTC
GGAGATAGCTGGTTCTCCCCGAAAACTATTTAGGTAGTGCCTCGAGCAAGACAC
TGATGGGGGTAAAGCACTGTTATGGCTAGGGGGTTATTGCAACTTACCAACCCAT
GGCAAACTCAGAATACCATCAAGTGGTTCCTCGGGAGACAGACAGCGGGTGCTA
ACGTCCGTTGTCAAGAGGGAAACAACCCAGACCGCCGGCTAAGGTCCCAAATGA
TAGATTAAGTGGTAAACGAAGTGGGAAGGCACAGACAGCCAGGATGTTGGCTTA
GAAGCAGCCATCATTTAAAGAAAGCGTAATAGCTCACTGGTCGAGTCGTCCTGC
GCGGAAGATGTAACGGGGCTCAAATCTATAACCGAAGCTGCGGATGCCGGTTTA
CCGGCATGGTAGGGGAGCGTTCTGTAGGCTGATGAAGGTGCATTGTAAAGTGTG
CTGGAGGTATCAGAAGTGCGAATGTTGACATGAGTAGCGATAAAGCGGGTGAAA
AGCCCGCTCGCCGAAAGCCCAAGGTTTCCTACGCAACGTTCATCGGCGTAGGGT
GAGTCGGCCCCTAAGGCGAGGCAGAAATGCGTAGTCGATGGGAAACAGGTTAAT
ATTCCTGTACTTGATTCAAATGCGATGTGGGGACGGAGAAGGTTAGGTTGGCAA
GCTGTTGGAATAGCTTGTTTAAGCCGGTAGGTGGAAGACTTAGGCAAATCCGGG
TTTTCTTAACACCGAGAAGTGATGACGAGTGTCTACGGACACGAAGCAACCGAT
ACCACGCTTCCAGGAAAAGCCACTAAGCTTCAGTTTGAATCGAACCGTACCGCA
AACCGACACAGGTGGGCAGGATGAGAATTCTAAGGCGCTTGAGAGAACTCGGG
AGAAGGAACTCGGCAAATTGATACCGTAACTTCGGGAGAAGGTATGCCCTCTAA
GGTTAAGGACTTGCTCCGTAAGCCCCGGAGGGTCGCAGAGAATAGGTGGCTGCG
ACTGTTTATTAAAAACACAGCACTCTGCCAACACGAAAGTGGACGTATAGGGTG
TGACGCCTGCCCGGTGCCGGAAGGTTAATTGAAGATGTGCAAGCATCGGATCGA
AGCCCCGGTAAACGGCGGCCGTAACTATAACGGTCCTAAGGTAGCGAAATTCCT
TGTCGGGTAAGTTCCGACCCGCACGAATGGCGTAACGATGGCCACACTGTCTCCT
CCCGAGACTCAGCGAAGTTGAAGTGGTTGTGAAGATGCAATCTACCCGCTGCTA
GACGGAAAGACCCCGTGAACCTTTACTGTAGCTTTGCATTGGACTTTGAAGTCAC
TTGTGTAGGATAGGTGGGAGGCTTGGAAGCAGAGACGCCAGTCTCTGTGGAGTC
GTCCTTGAAATACCACCCTGGTGTCTTTGAGGTTCTAACCCAGACCCGTCATCCG
GGTCGGGGACCGTGCATGGTAGGCAGTTTGACTGGGGCGGTCTCCTCCCAAAGC
GTAACGGAGGAGTTCGAAGGTTACCTAGGTCCGGTCGGAAATCGGACTGATAGT
GCAATGGCAAAAGGTAGCTTAACTGCGAGACCGACAAGTCGGGCAGGTGCGAA
AGCAGGACATAGTGATCCGGTGGTTCTGTATGGAAGGGCCATCGCTCAACGGAT
AAAAGGTACTCCGGGGATAACAGGCTGATTCCGCCCAAGAGTTCATATCGACGG
CGGAGTTTGGCACCTCGATGTCGGCTCATCACATCCTGGGGCTGTAGTCGGTCCC
AAGGGTATGGCTGTTCGCCATTTAAAGTGGTACGTGAGCTGGGTTTAAAACGTCG
TGAGACAGTTTGGTCCCTATCTGCAGTGGGCGTTGGAAGTTTGACGGGGGCTGCT
CCTAGTACGAGAGGACCGGAGTGGACGAACCTCTGGTGTACCGGTTGTAACGCC
AGTTGCATAGCCGGGTAGCTAAGTTCGGAAGAGATAAGCGCTGAAAGCATCTAA
GCGCGAAACTCGCCTGAAGATGAGACTTCCCTTGCGGTTTAACCGCACTAAAGG
GTCGTTCGAGACCAGGACGTTGATAGGTGGGGTGTGGAAGCGCGGTAACGCGTG
AAGCTAACCCATACTAATTGCCCGTGAGGCTTGACTCT

RNA: SEQ ID NO: 343

UGAAAUGAUAGAGUCAAGUGAAUAAGUGCAUCAGGCGGAUGCCUUGGCGAUG
AUAGGCGACGAAGGACGUGUAAGCCUGCGAAAAGCGCGGGGGAGCUGGCAAU
AAAGCAAUGAUCCCGCGGUGUCCGAAUGGGGAAACCCACUGCAUUCUGUGCA
GUAUCCUAAGUUGAAUACAUAGGCUUAGAGAAGCGAACCCGGAGAACUGACC
CAUCUAAGUACCCGGAGGAAAAGAAAUCAACCGAGAUUCCGCAAGUAGUGGC
GAGCGAACGCGGAGGAGCCUGUACGUAAUAACUGUCGAGGUAGAAGAACAAG
CUGGGAAGCUUGACCAUAGCGGGUGACAGUCCCGUAUUCGAAAUCUCAACAG
CGGUACUAAGCGUACGAAAAGUAGGGCGGGACACGUGAAAUCCUGUCUGAAU
AUGGGGGGACCAUCCUCCAAGGCUAAAUACUCAUCAUCGACCGAUAGUGAACC
AGUACCGUGAGGGAAAGGCGAAAAGAACCCCGGGAGGGGAGUGAAACAGAAC
CUGAAACCUGAUGCAUACAAACAGUGGGAGCGCCCUAGUGGUGUGACUGCGU
ACCUUUUGUAUAAUGGGUCAACGACUUACAUUCAGUAGCGAGCUUAACCGGA
UAGGGGAGGCGUAGGGAAACCGAGUCUUAAUAGGGCGAUGAGUUGCUGGGUG
UAGACCCGAAACCGAGUGAUCUAUCCAUGGCCAGGUUGAAGGUGCCGUAACA
GGUACUGGAGGACCGAACCCACGCAUGUUGCAAAAUGCGGGGAUGAGCUGUG
GGUAGGGGUGAAAGGCUAAACAAACUCGGAGAUAGCUGGUUCUCCCCGAAAA
CUAUUUAGGUAGUGCCUCGAGCAAGACACUGAUGGGGGUAAAGCACUGUUAU
GGCUAGGGGGUUAUUGCAACUUACCAACCCAUGGCAAACUCAGAAUACCAUC
AAGUGGUUCCUCGGGAGACAGACAGCGGGUGCUAACGUCCGUUGUCAAGAGG
GAAACAACCCAGACCGCCGGCUAAGGUCCCAAAUGAUAGAUUAAGUGGUAAA
CGAAGUGGGAAGGCACAGACAGCCAGGAUGUUGGCUUAGAAGCAGCCAUCAU
UUAAAGAAAGCGUAAUAGCUCACUGGUCGAGUCGUCCUGCGCGGAAGAUGUA
ACGGGGCUCAAAUCUAUAACCGAAGCUGCGGAUGCCGGUUUACCGGCAUGGU
AGGGGAGCGUUCUGUAGGCUGAUGAAGGUGCAUUGUAAAGUGUGCUGGAGGU
AUCAGAAGUGCGAAUGUUGACAUGAGUAGCGAUAAAGCGGGUGAAAAGCCCG
CUCGCCGAAAGCCCAAGGUUUCCUACGCAACGUUCAUCGGCGUAGGGUGAGUC
GGCCCCUAAGGCGAGGCAGAAAUGCGUAGUCGAUGGGAAACAGGUUAAUAUU
CCUGUACUUGAUUCAAAUGCGAUGUGGGGACGGAGAAGGUUAGGUUGGCAAG
CUGUUGGAAUAGCUUGUUUAAGCCGGUAGGUGGAAGACUUAGGCAAAUCCGG
GUUUUCUUAACACCGAGAAGUGAUGACGAGUGUCUACGGACACGAAGCAACC
GAUACCACGCUUCCAGGAAAAGCCACUAAGCUUCAGUUUGAAUCGAACCGUAC
CGCAAACCGACACAGGUGGGCAGGAUGAGAAUUCUAAGGCGCUUGAGAGAAC
UCGGGAGAAGGAACUCGGCAAAUUGAUACCGUAACUUCGGGAGAAGGUAUGC
CCUCUAAGGUUAAGGACUUGCUCCGUAAGCCCCGGAGGGUCGCAGAGAAUAG
GUGGCUGCGACUGUUUAUUAAAAACACAGCACUCUGCCAACACGAAAGUGGA
CGUAUAGGGUGUGACGCCUGCCCGGUGCCGGAAGGUUAAUUGAAGAUGUGCA
AGCAUCGGAUCGAAGCCCCGGUAAACGGCGGCCGUAACUAUAACGGUCCUAAG
GUAGCGAAAUUCCUUGUCGGGUAAGUUCCGACCCGCACGAAUGGCGUAACGA
UGGCCACACUGUCUCCUCCCGAGACUCAGCGAAGUUGAAGUGGUUGUGAAGA
UGCAAUCUACCCGCUGCUAGACGGAAAGACCCCGUGAACCUUUACUGUAGCUU
UGCAUUGGACUUUGAAGUCACUUGUGUAGGAUAGGUGGGAGGCUUGGAAGCA
GAGACGCCAGUCUCUGUGGAGUCGUCCUUGAAAUACCACCCUGGUGUCUUUG
AGGUUCUAACCCAGACCCGUCAUCCGGGUCGGGGACCGUGCAUGGUAGGCAGU
UUGACUGGGGCGGUCUCCUCCCAAAGCGUAACGGAGGAGUUCGAAGGUUACC
UAGGUCCGGUCGGAAAUCGGACUGAUAGUGCAAUGGCAAAAGGUAGCUUAAC
UGCGAGACCGACAAGUCGGGCAGGUGCGAAAGCAGGACAUAGUGAUCCGGUG
GUUCUGUAUGGAAGGGCCAUCGCUCAACGGAUAAAAGGUACUCCGGGGAUAA
CAGGCUGAUUCCGCCCAAGAGUUCAUAUCGACGGCGGAGUUUGGCACCUCGAU
GUCGGCUCAUCACAUCCUGGGGCUGUAGUCGGUCCCAAGGGUAUGGCUGUUC
GCCAUUUAAAGUGGUACGUGAGCUGGGUUUAAAACGUCGUGAGACAGUUUGG
UCCCUAUCUGCAGUGGGCGUUGGAAGUUUGACGGGGGCUGCUCCUAGUACGA
GAGGACCGGAGUGGACGAACCUCUGGUGUACCGGUUGUAACGCCAGUUGCAU
AGCCGGGUAGCUAAGUUCGGAAGAGAUAAGCGCUGAAAGCAUCUAAGCGCGA
AACUCGCCUGAAGAUGAGACUUCCCUUGCGGUUUAACCGCACUAAAGGGUCG
UUCGAGACCAGGACGUUGAUAGGUGGGGUGUGGAAGCGCGGUAACGCGUGAA
GCUAACCCAUACUAAUUGCCCGUGAGGCUUGACUCU

cDNA: SEQ ID NO: 344

AGAGTCAAGCCTCACGGGCAATTAGTATGGGTTAGCTTCACGCGTTACCGCGCTT
CCACACCCCACCTATCAACGTCCTGGTCTCGAACGACCCTTTAGTGCGGTTAAAC
CGCAAGGGAAGTCTCATCTTCAGGCGAGTTTCGCGCTTAGATGCTTTCAGCGCTT
ATCTCTTCCGAACTTAGCTACCCGGCTATGCAACTGGCGTTACAACCGGTACACC
AGAGGTTCGTCCACTCCGGTCCTCTCGTACTAGGAGCAGCCCCCGTCAAACTTCC
AACGCCCACTGCAGATAGGGACCAAACTGTCTCACGACGTTTTAAACCCAGCTC
ACGTACCACTTTAAATGGCGAACAGCCATACCCTTGGGACCGACTACAGCCCCA
GGATGTGATGAGCCGACATCGAGGTGCCAAACTCCGCCGTCGATATGAACTCTT
GGGCGGAATCAGCCTGTTATCCCCGGAGTACCTTTTATCCGTTGAGCGATGGCCC
TTCCATACAGAACCACCGGATCACTATGTCCTGCTTTCGCACCTGCCCGACTTGT
CGGTCTCGCAGTTAAGCTACCTTTTGCCATTGCACTATCAGTCCGATTTCCGACC
GGACCTAGGTAACCTTCGAACTCCTCCGTTACGCTTTGGGAGGAGACCGCCCCA
GTCAAACTGCCTACCATGCACGGTCCCCGACCCGGATGACGGGTCTGGGTTAGA
ACCTCAAAGACACCAGGGTGGTATTTCAAGGACGACTCCACAGAGACTGGCGTC
TCTGCTTCCAAGCCTCCCACCTATCCTACACAAGTGACTTCAAAGTCCAATGCAA
AGCTACAGTAAAGGTTCACGGGGTCTTTCCGTCTAGCAGCGGGTAGATTGCATCT
TCACAACCACTTCAACTTCGCTGAGTCTCGGGAGGAGACAGTGTGGCCATCGTTA
CGCCATTCGTGCGGGTCGGAACTTACCCGACAAGGAATTTCGCTACCTTAGGACC
GTTATAGTTACGGCCGCCGTTTACCGGGGCTTCGATCCGATGCTTGCACATCTTC
AATTAACCTTCCGGCACCGGGCAGGCGTCACACCCTATACGTCCACTTTCGTGTT
GGCAGAGTGCTGTGTTTTTAATAAACAGTCGCAGCCACCTATTCTCTGCGACCCT
CCGGGGCTTACGGAGCAAGTCCTTAACCTTAGAGGGCATACCTTCTCCCGAAGTT
ACGGTATCAATTTGCCGAGTTCCTTCTCCCGAGTTCTCTCAAGCGCCTTAGAATTC
TCATCCTGCCCACCTGTGTCGGTTTGCGGTACGGTTCGATTCAAACTGAAGCTTA
GTGGCTTTTCCTGGAAGCGTGGTATCGGTTGCTTCGTGTCCGTAGACACTCGTCA
TCACTTCTCGGTGTTAAGAAAACCCGGATTTGCCTAAGTCTTCCACCTACCGGCT
TAAACAAGCTATTCCAACAGCTTGCCAACCTAACCTTCTCCGTCCCCACATCGCA
TTTGAATCAAGTACAGGAATATTAACCTGTTTCCCATCGACTACGCATTTCTGCC
TCGCCTTAGGGGCCGACTCACCCTACGCCGATGAACGTTGCGTAGGAAACCTTG
GGCTTTCGGCGAGCGGGCTTTTCACCCGCTTTATCGCTACTCATGTCAACATTCG
CACTTCTGATACCTCCAGCACACTTTACAATGCACCTTCATCAGCCTACAGAACG
CTCCCCTACCATGCCGGTAAACCGGCATCCGCAGCTTCGGTTATAGATTTGAGCC
CCGTTACATCTTCCGCGCAGGACGACTCGACCAGTGAGCTATTACGCTTTCTTTA
AATGATGGCTGCTTCTAAGCCAACATCCTGGCTGTCTGTGCCTTCCCACTTCGTTT
ACCACTTAATCTATCATTTGGGACCTTAGCCGGCGGTCTGGGTTGTTTCCCTCTTG
ACAACGGACGTTAGCACCCGCTGTCTGTCTCCCGAGGAACCACTTGATGGTATTC
TGAGTTTGCCATGGGTTGGTAAGTTGCAATAACCCCCTAGCCATAACAGTGCTTT
ACCCCCATCAGTGTCTTGCTCGAGGCACTACCTAAATAGTTTTCGGGGAGAACCA
GCTATCTCCGAGTTTGTTTAGCCTTTCACCCCTACCCACAGCTCATCCCCGCATTT
TGCAACATGCGTGGGTTCGGTCCTCCAGTACCTGTTACGGCACCTTCAACCTGGC
CATGGATAGATCACTCGGTTTCGGGTCTACACCCAGCAACTCATCGCCCTATTAA
GACTCGGTTTCCCTACGCCTCCCCTATCCGGTTAAGCTCGCTACTGAATGTAAGT
CGTTGACCCATTATACAAAAGGTACGCAGTCACACCACTAGGGCGCTCCCACTG
TTTGTATGCATCAGGTTTCAGGTTCTGTTTCACTCCCCTCCCGGGGTTCTTTTCGC
CTTTCCCTCACGGTACTGGTTCACTATCGGTCGATGATGAGTATTTAGCCTTGGA
GGATGGTCCCCCCATATTCAGACAGGATTTCACGTGTCCCGCCCTACTTTTCGTA
CGCTTAGTACCGCTGTTGAGATTTCGAATACGGGACTGTCACCCGCTATGGTCAA
GCTTCCCAGCTTGTTCTTCTACCTCGACAGTTATTACGTACAGGCTCCTCCGCGTT
CGCTCGCCACTACTTGCGGAATCTCGGTTGATTTCTTTTCCTCCGGGTACTTAGAT
GGGTCAGTTCTCCGGGTTCGCTTCTCTAAGCCTATGTATTCAACTTAGGATACTG
CACAGAATGCAGTGGGTTTCCCCATTCGGACACCGCGGGATCATTGCTTTATTGC
CAGCTCCCCCGCGCTTTTCGCAGGCTTACACGTCCTTCGTCGCCTATCATCGCCA
AGGCATCCGCCTGATGCACTTATTCACTTGACTCTATCATTTCA

### 8. NGO_r12: NC_002946.2:c1878117-1876573

DNA (- strand): SEQ ID NO: 10

TGAACATAAGAGTTTGATCCTGGCTCAGATTGAACGCTGGCGGCATGCTTTACAC
ATGCAAGTCGGACGGCAGCACAGGGAAGCTTGCTTCTCGGGTGGCGAGTGGCGA
ACGGGTGAGTAACATATCGGAACGTACCGGGTAGCGGGGGATAACTGATCGAAA
GATCAGCTAATACCGCATACGTCTTGAGAGGGAAAGCAGGGGACCTTCGGGCCT
TGCGCTATCCGAGCGGCCGATATCTGATTAGCTGGTTGGCGGGGTAAAGGCCCA
CCAAGGCGACGATCAGTAGCGGGTCTGAGAGGATGATCCGCCACACTGGGACTG
AGACACGGCCCAGACTCCTACGGGAGGCAGCAGTGGGGAATTTTGGACAATGGG
CGCAAGCCTGATCCAGCCATGCCGCGTGTCTGAAGAAGGCCTTCGGGTTGTAAA
GGACTTTTGTCAGGGAAGAAAAGGCCGTTGCCAATATCGGCGGCCGATGACGGT
ACCTGAAGAATAAGCACCGGCTAACTACGTGCCAGCAGCCGCGGTAATACGTAG
GGTGCGAGCGTTAATCGGAATTACTGGGCGTAAAGCGGGCGCAGACGGTTACTT
AAGCAGGATGTGAAATCCCCGGGCTCAACCCGGGAACTGCGTTCTGAACTGGGT
GACTCGAGTGTGTCAGAGGGAGGTGGAATTCCACGTGTAGCAGTGAAATGCGTA
GAGATGTGGAGGAATACCGATGGCGAAGGCAGCCTCCTGGGATAACACTGACGT
TCATGTCCGAAAGCGTGGGTAGCAAACAGGATTAGATACCCTGGTAGTCCACGC
CCTAAACGATGTCAATTAGCTGTTGGGCAACTTGATTGCTTGGTAGCGTAGCTAA
CGCGTGAAATTGACCGCCTGGGGAGTACGGTCGCAAGATTAAAACTCAAAGGAA
TTGACGGGGACCCGCACAAGCGGTGGATGATGTGGATTAATTCGATGCAACGCG
AAGAACCTTACCTGGTTTTGACATGTGCGGAATCCTCCGGAGACGGAGGAGTGC
CTTCGGGAGCCGTAACACAGGTGCTGCATGGCTGTCGTCAGCTCGTGTCGTGAGA
TGTTGGGTTAAGTCCCGCAACGAGCGCAACCCTTGTCATTAGTTGCCATCATTCG
GTTGGGCACTCTAATGAGACTGCCGGTGACAAGCCGGAGGAAGGTGGGGATGAC
GTCAAGTCCTCATGGCCCTTATGACCAGGGCTTCACACGTCATACAATGGTCGGT
ACAGAGGGTAGCCAAGCCGCGAGGCGGAGCCAATCTCACAAAACCGATCGTAG
TCCGGATTGCACTCTGCAACTCGAGTGCATGAAGTCGGAATCGCTAGTAATCGCA
GGTCAGCATACTGCGGTGAATACGTTCCCGGGTCTTGTACACACCGCCCGTCACA
CCATGGGAGTGGGGGATACCAGAAGTAGGTAGGGTAACCGCAAGGAGTCCGCTT
ACCACGGTATGCTTCATGACTGGGGTGAAGTCGTAACAAGGTAGCCGTAGGGGA
ACCTGCGGCTGGATCACCTCCTTTCTA

RNA: SEQ ID NO: 11

UGAACAUAAGAGUUUGAUCCUGGCUCAGAUUGAACGCUGGCGGCAUGCUUUA
CACAUGCAAGUCGGACGGCAGCACAGGGAAGCUUGCUUCUCGGGUGGCGAGU
GGCGAACGGGUGAGUAACAUAUCGGAACGUACCGGGUAGCGGGGGAUAACUG
AUCGAAAGAUCAGCUAAUACCGCAUACGUCUUGAGAGGGAAAGCAGGGGACC
UUCGGGCCUUGCGCUAUCCGAGCGGCCGAUAUCUGAUUAGCUGGUUGGCGGG
GUAAAGGCCCACCAAGGCGACGAUCAGUAGCGGGUCUGAGAGGAUGAUCCGC
CACACUGGGACUGAGACACGGCCCAGACUCCUACGGGAGGCAGCAGUGGGGAA
UUUUGGACAAUGGGCGCAAGCCUGAUCCAGCCAUGCCGCGUGUCUGAAGAAG
GCCUUCGGGUUGUAAAGGACUUUUGUCAGGGAAGAAAAGGCCGUUGCCAAUA
UCGGCGGCCGAUGACGGUACCUGAAGAAUAAGCACCGGCUAACUACGUGCCAG
CAGCCGCGGUAAUACGUAGGGUGCGAGCGUUAAUCGGAAUUACUGGGCGUAA
AGCGGGCGCAGACGGUUACUUAAGCAGGAUGUGAAAUCCCCGGGCUCAACCCG
GGAACUGCGUUCUGAACUGGGUGACUCGAGUGUGUCAGAGGGAGGUGGAAUU
CCACGUGUAGCAGUGAAAUGCGUAGAGAUGUGGAGGAAUACCGAUGGCGAAG
GCAGCCUCCUGGGAUAACACUGACGUUCAUGUCCGAAAGCGUGGGUAGCAAA
CAGGAUUAGAUACCCUGGUAGUCCACGCCCUAAACGAUGUCAAUUAGCUGUU
GGGCAACUUGAUUGCUUGGUAGCGUAGCUAACGCGUGAAAUUGACCGCCUGG
GGAGUACGGUCGCAAGAUUAAAACUCAAAGGAAUUGACGGGGACCCGCACAA
GCGGUGGAUGAUGUGGAUUAAUUCGAUGCAACGCGAAGAACCUUACCUGGUU
UUGACAUGUGCGGAAUCCUCCGGAGACGGAGGAGUGCCUUCGGGAGCCGUAA
CACAGGUGCUGCAUGGCUGUCGUCAGCUCGUGUCGUGAGAUGUUGGGUUAAG
UCCCGCAACGAGCGCAACCCUUGUCAUUAGUUGCCAUCAUUCGGUUGGGCACU
CUAAUGAGACUGCCGGUGACAAGCCGGAGGAAGGUGGGGAUGACGUCAAGUC
CUCAUGGCCCUUAUGACCAGGGCUUCACACGUCAUACAAUGGUCGGUACAGAG
GGUAGCCAAGCCGCGAGGCGGAGCCAAUCUCACAAAACCGAUCGUAGUCCGGA
UUGCACUCUGCAACUCGAGUGCAUGAAGUCGGAAUCGCUAGUAAUCGCAGGU
CAGCAUACUGCGGUGAAUACGUUCCCGGGUCUUGUACACACCGCCCGUCACAC
CAUGGGAGUGGGGGAUACCAGAAGUAGGUAGGGUAACCGCAAGGAGUCCGCU
UACCACGGUAUGCUUCAUGACUGGGGUGAAGUCGUAACAAGGUAGCCGUAGG
GGAACCUGCGGCUGGAUCACCUCCUUUCUA

cDNA: SEQ ID NO: 12

TAGAAAGGAGGTGATCCAGCCGCAGGTTCCCCTACGGCTACCTTGTTACGACTTC
ACCCCAGTCATGAAGCATACCGTGGTAAGCGGACTCCTTGCGGTTACCCTACCTA
CTTCTGGTATCCCCCACTCCCATGGTGTGACGGGCGGTGTGTACAAGACCCGGGA
ACGTATTCACCGCAGTATGCTGACCTGCGATTACTAGCGATTCCGACTTCATGCA
CTCGAGTTGCAGAGTGCAATCCGGACTACGATCGGTTTTGTGAGATTGGCTCCGC
CTCGCGGCTTGGCTACCCTCTGTACCGACCATTGTATGACGTGTGAAGCCCTGGT
CATAAGGGCCATGAGGACTTGACGTCATCCCCACCTTCCTCCGGCTTGTCACCGG
CAGTCTCATTAGAGTGCCCAACCGAATGATGGCAACTAATGACAAGGGTTGCGC
TCGTTGCGGGACTTAACCCAACATCTCACGACACGAGCTGACGACAGCCATGCA
GCACCTGTGTTACGGCTCCCGAAGGCACTCCTCCGTCTCCGGAGGATTCCGCACA
TGTCAAAACCAGGTAAGGTTCTTCGCGTTGCATCGAATTAATCCACATCATCCAC
CGCTTGTGCGGGTCCCCGTCAATTCCTTTGAGTTTTAATCTTGCGACCGTACTCCC
CAGGCGGTCAATTTCACGCGTTAGCTACGCTACCAAGCAATCAAGTTGCCCAAC
AGCTAATTGACATCGTTTAGGGCGTGGACTACCAGGGTATCTAATCCTGTTTGCT
ACCCACGCTTTCGGACATGAACGTCAGTGTTATCCCAGGAGGCTGCCTTCGCCAT
CGGTATTCCTCCACATCTCTACGCATTTCACTGCTACACGTGGAATTCCACCTCCC
TCTGACACACTCGAGTCACCCAGTTCAGAACGCAGTTCCCGGGTTGAGCCCGGG
GATTTCACATCCTGCTTAAGTAACCGTCTGCGCCCGCTTTACGCCCAGTAATTCC
GATTAACGCTCGCACCCTACGTATTACCGCGGCTGCTGGCACGTAGTTAGCCGGT
GCTTATTCTTCAGGTACCGTCATCGGCCGCCGATATTGGCAACGGCCTTTTCTTCC
CTGACAAAAGTCCTTTACAACCCGAAGGCCTTCTTCAGACACGCGGCATGGCTG
GATCAGGCTTGCGCCCATTGTCCAAAATTCCCCACTGCTGCCTCCCGTAGGAGTC
TGGGCCGTGTCTCAGTCCCAGTGTGGCGGATCATCCTCTCAGACCCGCTACTGAT
CGTCGCCTTGGTGGGCCTTTACCCCGCCAACCAGCTAATCAGATATCGGCCGCTC
GGATAGCGCAAGGCCCGAAGGTCCCCTGCTTTCCCTCTCAAGACGTATGCGGTAT
TAGCTGATCTTTCGATCAGTTATCCCCCGCTACCCGGTACGTTCCGATATGTTACT
CACCCGTTCGCCACTCGCCACCCGAGAAGCAAGCTTCCCTGTGCTGCCGTCCGAC
TTGCATGTGTAAAGCATGCCGCCAGCGTTCAATCTGAGCCAGGATCAAACTCTTA
TGTTCA

1-52. (canceled)

53. A method to detect a transcript of an N. gonorrhoeae, the method comprises
quantitatively detecting a transcript expression value of an RNA marker of N. gonorrhoeae , in the N. gonorrhoeae following contacting of the N. gonorrhoeae with an antibiotic to obtain an antibiotic treated transcript expression value for the RNA marker of N. gonorrhoeae,
wherein the RNA marker of N. gonorrhoeae is selected from:
a transcript of N. gonorrhoeae gene having locus tag NGO0340,
a transcript of N. gonorrhoeae gene having locus tag NGO1837,
a transcript of N. gonorrhoeae gene having locus tag NGO1843,
a transcript of N. gonorrhoeae gene having locus tag NGO2024,
a transcript of N. gonorrhoeae gene having locus tag NGO1845,
a transcript of N. gonorrhoeae gene having locus tag NGO1677,
a transcript of N. gonorrhoeae gene having locus tag NGO1844,
a transcript of N. gonorrhoeae gene having locus tag NGO0171 ,
a transcript of N. gonorrhoeae gene having locus tag NGO1834,
a transcript of N. gonorrhoeae gene having locus tag NGO0172,
a transcript of N. gonorrhoeae gene having locus tag NGO1835,
a transcript of N. gonorrhoeae gene having locus tag NGO1673,
a transcript of N. gonorrhoeae gene having locus tag NGO1833,
a transcript of N. gonorrhoeae gene having locus tag NGO2173,
a transcript of N. gonorrhoeae gene having locus tag NGO0604,
a transcript of N. gonorrhoeae gene having locus tag NGO0016,
a transcript of N. gonorrhoeae gene having locus tag NGO2174,
a transcript of N. gonorrhoeae gene having locus tag NGO2164,
a transcript of N. gonorrhoeae gene having locus tag NGO1676,
a transcript of N. gonorrhoeae gene having locus tag NGO1679,
a transcript of N. gonorrhoeae gene having locus tag NGO1658,
a transcript of N. gonorrhoeae gene having locus tag NGO1440,
a transcript of N. gonorrhoeae gene having locus tag NGO0174,
a transcript of N. gonorrhoeae gene having locus tag NGO0173,
a transcript of N. gonorrhoeae gene having locus tag NGO0592,
a transcript of N. gonorrhoeae gene having locus tag NGO1680,
a transcript of N. gonorrhoeae gene having locus tag NGO0620,
a transcript of N. gonorrhoeae gene having locus tag NGO1659,
a transcript of N. gonorrhoeae gene having locus tag NGO1291,
a transcript of N. gonorrhoeae gene having locus tag NGO0648,
a transcript of N. gonorrhoeae gene having locus tag NGO0593,
a transcript of N. gonorrhoeae gene having locus tag NGO1804,
a transcript of N. gonorrhoeae gene having locus tag NGO0618,
a transcript of N. gonorrhoeae gene having locus tag NGO0619,
a transcript of N. gonorrhoeae gene having locus tag NGO1812,
a transcript of N. gonorrhoeae gene having locus tag NGO1890,
a transcript of N. gonorrhoeae gene having locus tag NGO2098,
a transcript of N. gonorrhoeae gene having locus tag NGO2100,
a transcript tRNA having a GenelD A9Y61 RS02445 or NGO t12,
a transcript tRNA having a GenelD A9Y61 RS04515 or NGO t15,
a transcript tRNA having a GenelD A9Y61 RS04510 or NGO t14,
a transcript tRNA having a GenelD A9Y61 RS09170 or NGO t37,
a transcript tRNA having a GenelD A9Y61 RS00075 or NGO t01, and
a sequence having at least 80% identity with any one of the transcripts.

54. The method of claim 53, the method further comprising
detecting whether there is a downshift in the transcript expression value of the RNA marker of N. gonorrhoeae following the contacting by comparing the antibiotic treated transcript expression value with an untreated marker expression value of the RNA marker of N. gonorrhoeae.

55. The method of claim 54, wherein the reference expression value of the RNA marker of N. gonorrhoeae is a control transcript expression value obtained by quantitatively detecting the RNA of N. gonorrhoeae in a control sample of the isolate or specimen not treated with the antibiotic.

56. The method of claim 55, wherein the quantitatively detecting a transcript expression value of an RNA marker of N. gonorrhoeae is performed by
contacting a sample of an isolate or specimen comprising the N. gonorrhoeae with an antibiotic to obtain an antibiotic treated sample,
quantitatively detecting a transcript expression value of a RNA marker of N. gonorrhoeae in the antibiotic treated sample, to provide an antibiotic treated transcript expression value for the RNA marker of N. gonorrhoeae,
quantitatively detecting the transcript expression value of the RNA marker of N. gonorrhoeae in a control sample of the isolate or specimen comprising the N. gonorrhoeae, to provide a control transcript expression value of the RNA marker of N. gonorrhoeae herein described; and
detecting whether there is a downshift of the transcript of the RNA marker of N. gonorrhoeae herein described in the treated sample with respect to the control sample.

57. The method of claim 54, further comprising normalizing the antibiotic treated transcript expression value, the control transcript expression value and/or the related ratio, before detecting whether there is a downshift.

58. The method of claim 57, wherein the normalizing is performed with a reference measurement selected from expression value of a reference RNA, preferably a low variability and/or highly expressed RNA, DNA, number of cells, number of samples, effective amount of sample used and/or a related ratio.

59. The method of claim 54, wherein the downshift of the transcript presence is at least 1.5-fold.

60. The method of claim 54 , wherein the downshift of the transcript presence is at least 4-fold.

61. The method of claim 54 , wherein the downshift of the transcript presence is 6-fold or higher.

62. The method of claim 54, wherein contacting the sample with an antibiotic is performed for up to 15 minutes.

63. The method of claim 54, wherein contacting the sample with an antibiotic is performed for up to 10 minutes.

64. The method of claim 54, wherein contacting the sample with an antibiotic is performed for up to 5 minutes.

65. The method of claim 54, wherein the quantitatively detecting is performed by using a probe specific for any one of the RNA markers and/or a corresponding cDNA marker and/or a probe specific for a cDNA marker corresponding to any one of the RNA markers.

66. The method of claim 54, wherein the quantitatively detecting is performed in sample pretreated to enrich the RNA of N. gonorrhoeae and/or to remove of human RNA or RNA of other microorganisms in the sample.

67-124. (canceled)

125. A system for performing the method of claim 53, the system comprising a probe specific for an RNA marker, corresponding marker gene and/or corresponding cDNA and reagents for detecting said probe,
wherein the RNA marker is selected from
a transcript of N. gonorrhoeae gene having locus tag NGO0340,
a transcript of N. gonorrhoeae gene having locus tag NGO1837,
a transcript of N. gonorrhoeae gene having locus tag NGO1843,
a transcript of N. gonorrhoeae gene having locus tag NGO2024,
a transcript of N. gonorrhoeae gene having locus tag NGO1845,
a transcript of N. gonorrhoeae gene having locus tag NGO1677,
a transcript of N. gonorrhoeae gene having locus tag NGO1844,
a transcript of N. gonorrhoeae gene having locus tag NGO0171,
a transcript of N. gonorrhoeae gene having locus tag NGO1834,
a transcript of N. gonorrhoeae gene having locus tag NGO0172,
a transcript of N. gonorrhoeae gene having locus tag NGO1835,
a transcript of N. gonorrhoeae gene having locus tag NGO1673,
a transcript of N. gonorrhoeae gene having locus tag NGO1833,
a transcript of N. gonorrhoeae gene having locus tag NGO2173,
a transcript of N. gonorrhoeae gene having locus tag NGO0604,
a transcript of N. gonorrhoeae gene having locus tag NGO0016,
a transcript of N. gonorrhoeae gene having locus tag NGO2174,
a transcript of N. gonorrhoeae gene having locus tag NGO2164,
a transcript of N. gonorrhoeae gene having locus tag NGO1676,
a transcript of N. gonorrhoeae gene having locus tag NGO1679,
a transcript of N. gonorrhoeae gene having locus tag NGO1658,
a transcript of N. gonorrhoeae gene having locus tag NGO1440,
a transcript of N. gonorrhoeae gene having locus tag NGO0174,
a transcript of N. gonorrhoeae gene having locus tag NGO0173,
a transcript of N. gonorrhoeae gene having locus tag NGO0592,
a transcript of N. gonorrhoeae gene having locus tag NGO1680,
a transcript of N. gonorrhoeae gene having locus tag NGO0620,
a transcript of N. gonorrhoeae gene having locus tag NGO1659,
a transcript of N. gonorrhoeae gene having locus tag NGO1291 ,
a transcript of N. gonorrhoeae gene having locus tag NGO0648,
a transcript of N. gonorrhoeae gene having locus tag NGO0593,
a transcript of N. gonorrhoeae gene having locus tag NGO1804,
a transcript of N. gonorrhoeae gene having locus tag NGO0618,
a transcript of N. gonorrhoeae gene having locus tag NGO0619,
a transcript of N. gonorrhoeae gene having locus tag NGO1812,
a transcript of N. gonorrhoeae gene having locus tag NGO1890,
a transcript of N. gonorrhoeae gene having locus tag NGO2098,
a transcript of N. gonorrhoeae gene having locus tag NGO2100,
a transcript tRNA having a GenelD A9Y61 RS02445 or NGO t12,
a transcript tRNA having a GenelD A9Y61 RS04515 or NGO t15,
a transcript tRNA having a GenelD A9Y61 RS04510 or NGO t14,
a transcript tRNA having a GenelD A9Y61 RS09170 or NGO t37,
a transcript tRNA having a GenelD A9Y61 RS00075 or NGO t01 and
a sequence having at least 80% identity with any one of the transcripts.

126. The system of claim 125, wherein the probe comprises a probe specific for a transcript selected from any one of the RNA markers and/or a cDNA corresponding thereto, and a probe specific for a cDNA of any one of the RNA markers.

127. The system of claim 125, wherein the system comprises at least one probe specific for a transcript selected from
N. gonorrhoeae gene having locus tag NGO1812,
N. gonorrhoeae gene having locus tag NGO1680,
N. gonorrhoeae gene having locus tag NGO1291,
N. gonorrhoeae gene having locus tag NGO1673,
N. gonorrhoeae gene having locus tag NGO0592, and
N. gonorrhoeae gene having locus tag NGO0340
or for a corresponding cDNA.

128. The system of claim 125, wherein the system comprises at least one probe specific for a transcript selected from
N. gonorrhoeae gene having locus tag NGO1812, and/or
N. gonorrhoeae gene having locus tag NGO1680
or for a corresponding cDNA.

129. The system of claim 125, wherein the probe comprises primers configured to specifically hybridize with the transcript and/or the corresponding cDNA.

130. The system of claim 129, wherein the system comprises
a probe specific for a transcript of N. gonorrhoeae gene having locus tag NGO1812, the probe comprises a pair of primers having sequence GCTACGATTCTCCCGAATTTGCC (SEQ ID NO: 160) (CCGCCKACCAAACGGTGAAC (SEQ ID NO: 161),
a probe specific for a transcript of N. gonorrhoeae gene having locus tag NGO1680, the probe comprises a pair of primers having sequence TTGCCCAACTTGCAATCACG (SEQ ID NO: 162) and AGCACGCAAATCAGCCAATAC (SEQ ID NO: 163),
a probe specific for a transcript of N. gonorrhoeae gene having locus tag NGO1291, the probe comprises a pair of primers having sequence GCTTTGGAAAAAGCAGCCG (SEQ ID NO: 164) and GGTTTTGTTGTCGGTCAGGC (SEQ ID NO: 165),
a probe specific for a transcript of N. gonorrhoeae gene having locus tag NGO1673, the probe comprises a pair of primers having sequence GACTTTTGCCGCTGCTTTG (SEQ ID NO: 166) and GCGCATTATTCGTGTGCAG (SEQ ID NO: 167),
a probe specific for a transcript of N. gonorrhoeae gene having locus tag NGO0592, the probe comprises a pair of primers having sequence AAAGCCTTGGGTATTGCGG (SEQ ID NO: 168) and TGACCAAAGCAACCGGAAC (SEQ ID NO: 169), and/or
a probe specific for a transcript of N. gonorrhoeae gene having locus tag NGO0340, the probe comprises a pair of primers having sequence GAGGCTTCCCCCGTATTGAG (SEQ ID NO: 170) and TTCAAAAGCCGCTTCGTTCG (SEQ ID NO: 171).
